(19) United States
(12) Patent Application Publication
(10) Pub. No.: US 2025/0195485 A1
(43) Pub. Date: Jun. 19, 2025

(54) PROPIONIC ACID DERIVATIVE AND MEDICAL USE THEREOF

(71) Applicant: Xizang Haisco Pharmaceutical Co., Ltd., Naidong District, Lhoka, Tibet (CN)

(72) Inventors: Chen ZHANG, Chengdu City, Sichuan Province (CN); Ping HE, Chengdu City, Sichuan Province (CN); Haiqing HE, Chengdu City, Sichuan Province (CN); Yan YU, Chengdu City, Sichuan Province (CN); Pingming TANG, Chengdu City, Sichuan Province (CN); Zhaoli XUAN, Chengdu City, Sichuan Province (CN); Le WANG, Chengdu City, Sichuan Province (CN); Yao LI, Chengdu City, Sichuan Province (CN); Jia NI, Chengdu City, Sichuan Province (CN); Pangke YAN, Chengdu City, Sichuan Province (CN)

(73) Assignee: Xizang Haisco Pharmaceutical Co., Ltd., Naidong District, Lhoka, Tibet (CN)

(21) Appl. No.: 18/725,087

(22) PCT Filed: Dec. 21, 2022
(86) PCT No.: PCT/CN2022/140618

§ 371 Date: Jun. 27, 2024

(30) Foreign Application Priority Data
Dec. 27, 2021 (CN) 202111606278.X
Feb. 24, 2022 (CN) 202210170157.3
Sep. 9, 2022 (CN) 202211104699.7
Nov. 11, 2022 (CN) 202211391154.9

(51) Int. Cl. A61K 31/4436; A61K 31/4427; A61K 31/444; A61K 31/4995; A61K 31/506; C07D 213/64; C07D 401/06; C07D 401/12; C07D 401/14; C07D 409/12; C07D 417/12; C07D 471/04; C07D 487/08

(52) U.S. Cl. CPC A61K 31/4436; A61K 31/4427; A61K 31/444; A61K 31/4995; A61K 31/506; C07D 213/64; C07D 401/06; C07D 401/12; C07D 401/14; C07D 409/12; C07D 417/12; C07D 471/04; C07D 487/08

(57) ABSTRACT

A compound as represented by general formula (I) or a stereoisomer, a deuterated form, a solvate, a prodrug, a metabolite, a pharmaceutically acceptable salt or a co-crystal thereof; an intermediate thereof and a preparation method therefor; and the use thereof in the preparation of a drug for treating diseases related to an integrin α4β7 activity or expression level.

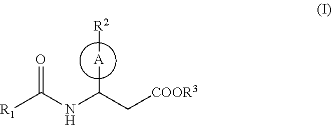

### CROSS REFERENCE TO RELATED APPLICATIONS

This application is a 35 U.S.C. § 371 National Stage of International Patent Application No. PCT/CN2022/140618, filed Dec. 21, 2022, designating the United States, which claims priority to and the benefits of Chinese Patent Application No. 202111606278.X, filed Dec. 27, 2021, Chinese Patent Application No. 202210170157.3, filed Feb. 24, 2022, Chinese Patent Application No. 202211104699.7, filed Sep. 9, 2022, and Chinese Patent Application No. 202211391154.9, filed Nov. 11, 2022, the disclosures of which are incorporated herein in their entirety by reference, and priority is claimed to each of the foregoing.

### TECHNICAL FIELD

The present disclosure relates to a compound as represented by general formula (I) or a stereoisomer, a deuterated form, a solvate, a prodrug, a metabolite, a pharmaceutically acceptable salt or a co-crystal thereof; an intermediate thereof and a preparation method therefor; and the use thereof in the preparation of a drug for the treatment of diseases related to an integrin α4β7 activity or expression level.

### BACKGROUND

The integrin family comprises dimers formed by two subunits: α (120-185 KD) and β (90-110 KD). Mammalian integrins involve 18 α-subunits and 8 β-subunits. More than 20 integrins can be formed from their various combinations. α4β7 is a member of the integrin family. It is currently established that inflammatory bowel diseases related to α4β7 include Crohn's disease, ulcerative colitis, etc. The main ligand of α4β7 is mucosal addressin cell adhesion molecule-1 (MAdCAM-1). MAdCAM-1 is a transmembrane glycoprotein molecule selectively expressed in the high endothelial veins of mucosal lymphoid organs and the veins of the intestinal lamina propria. In inflammation, a variety of cytokines can promote high expression of MAdCAM-1 by endothelial cells, and then MAdCAM-1 mediates the migration of α4β7-expressing leukocytes to the site of inflammation. Targeting either integrin α4β7 or MAdCAM-1 can reduce intestinal inflammation. There are currently no specific small-molecule compounds on the market that target α4β7-mediated inflammation. Natalizumab in clinical use is a humanized monoclonal antibody that targets the α4 subunit and is used primarily to treat multiple sclerosis and Crohn's disease. However, the side effect of PML (progressive multifocal leukoencephalopathy) has occurred during its clinical use. Therefore, there is a need to develop a small-molecule compound, which can inhibit integrin α4β7 protein, for use in the treatment of diseases related to an integrin α4β7 activity or expression level.

### SUMMARY

The present inventors have developed a safer integrin α4β7 inhibitor with a novel structure and improved efficacy. These compounds have good pharmacokinetic properties and high safety and can be used to treat diseases related to integrin α4β7, such as inflammatory bowel diseases.

An object of the present disclosure is to provide a compound capable of inhibiting integrin α4β7, or a stereoisomer, a deuterated form, a solvate, a prodrug, a metabolite, a pharmaceutically acceptable salt or a co-crystal thereof; an intermediate thereof and a preparation method therefor; and the use thereof in the preparation of a drug for treating diseases related to an integrin α4β7 activity or expression level.

The present disclosure provides a compound as represented by general formula (I) or a stereoisomer, a deuterated form, a solvate, a prodrug, a metabolite, a pharmaceutically acceptable salt or a co-crystal thereof, wherein

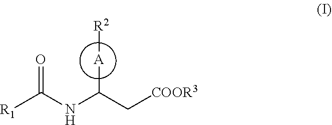

in some embodiments, R¹ is selected from —CHR^{1a}R^{1b} or —NR^{1a}R^{1b};
in some embodiments, R¹ is selected from

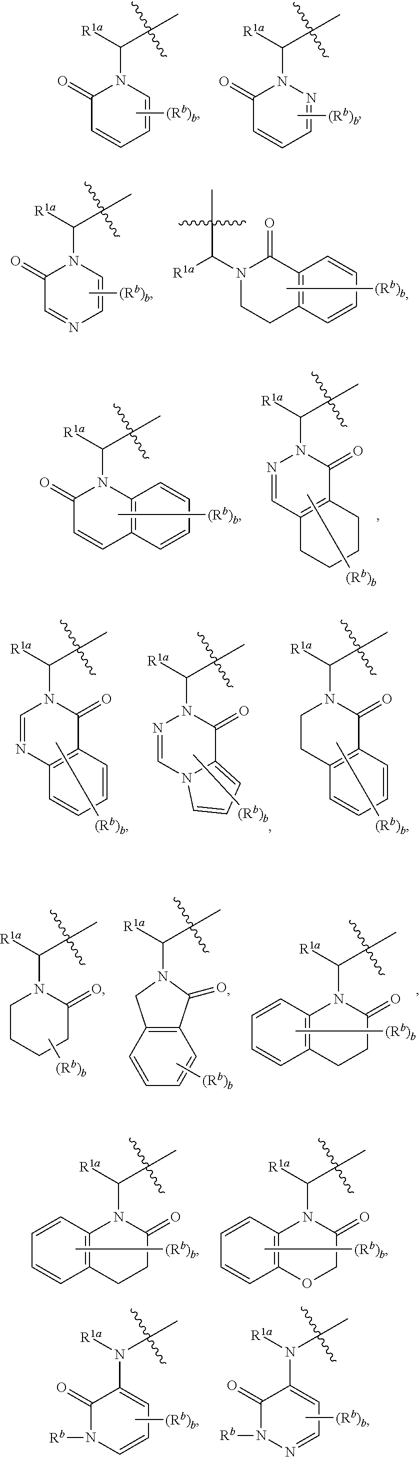

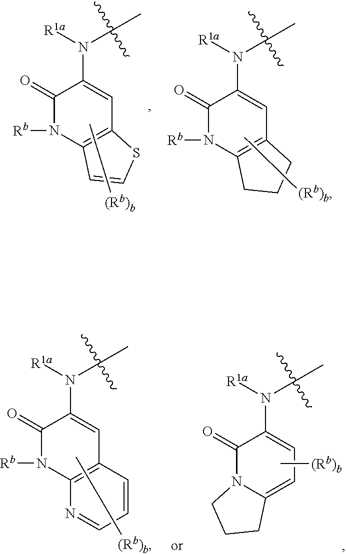

and b is selected from 0, 1, 2 or 3;
in some embodiments, R¹ is selected from

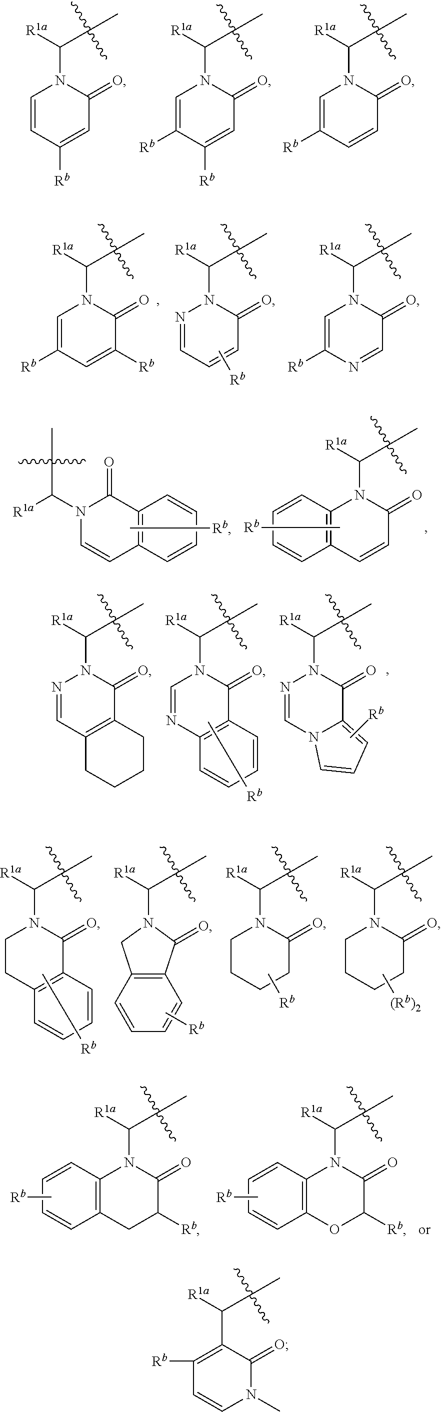

in some embodiments R¹ is selected from

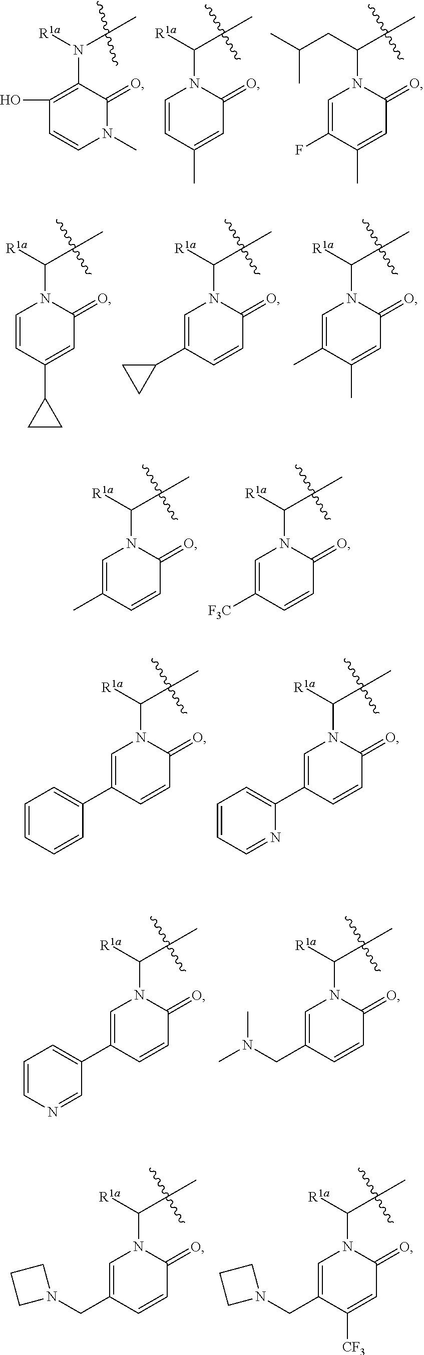

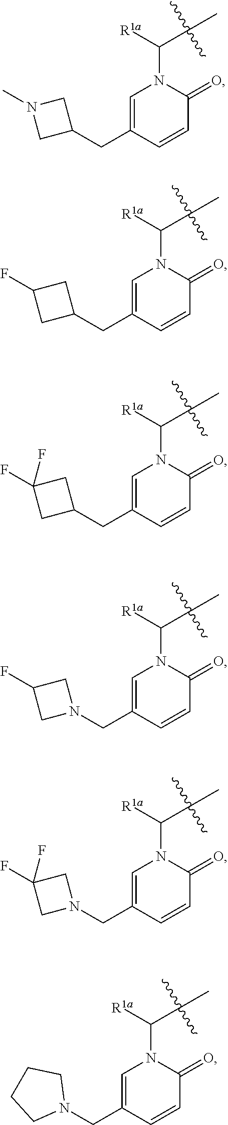

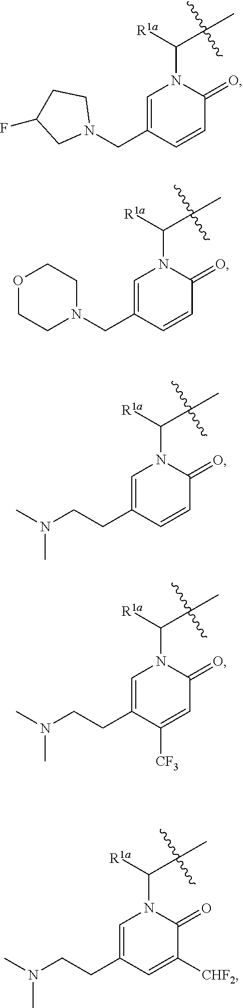

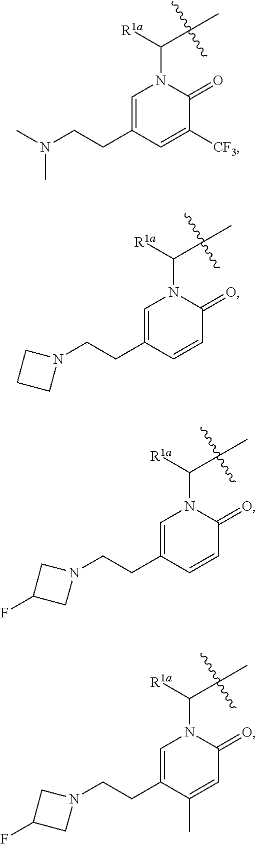

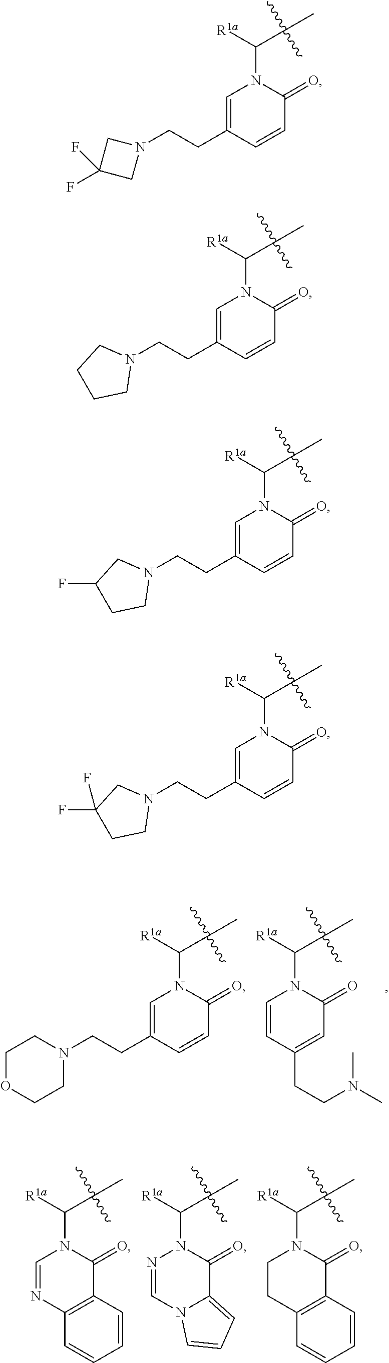

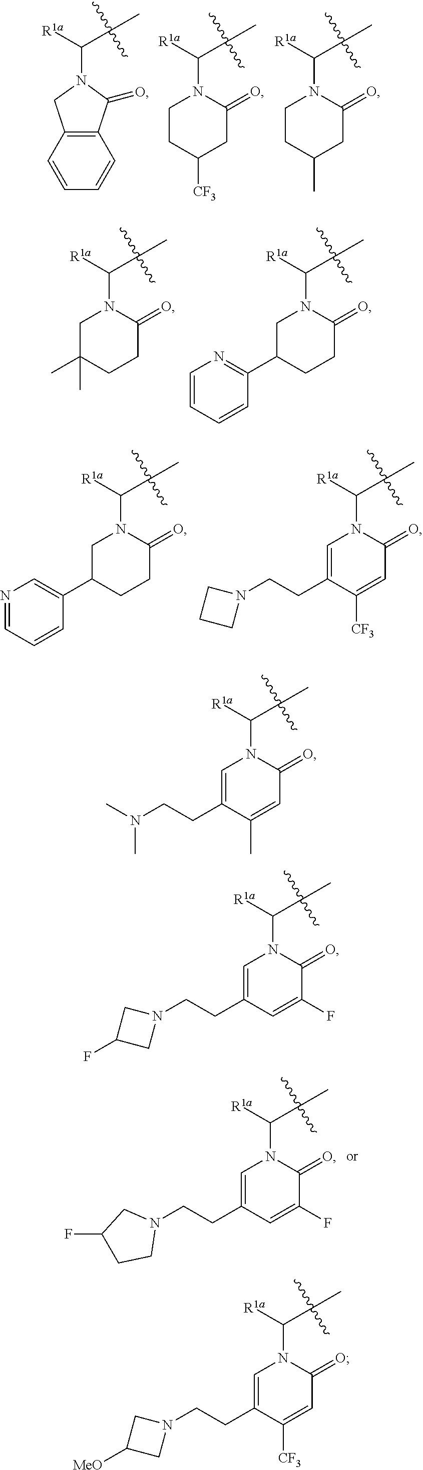

in some embodiments, R¹ is selected from

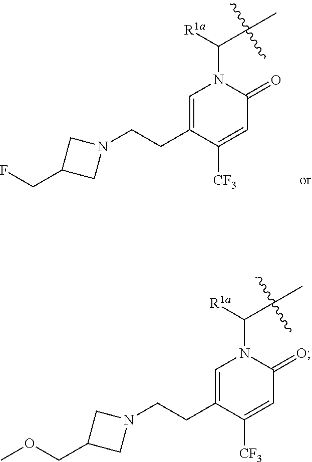

in some embodiments, R¹ is selected from

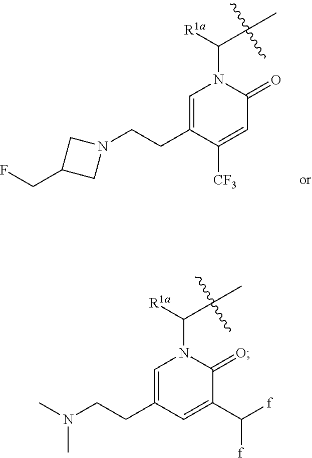

in some embodiments, R¹ is selected from

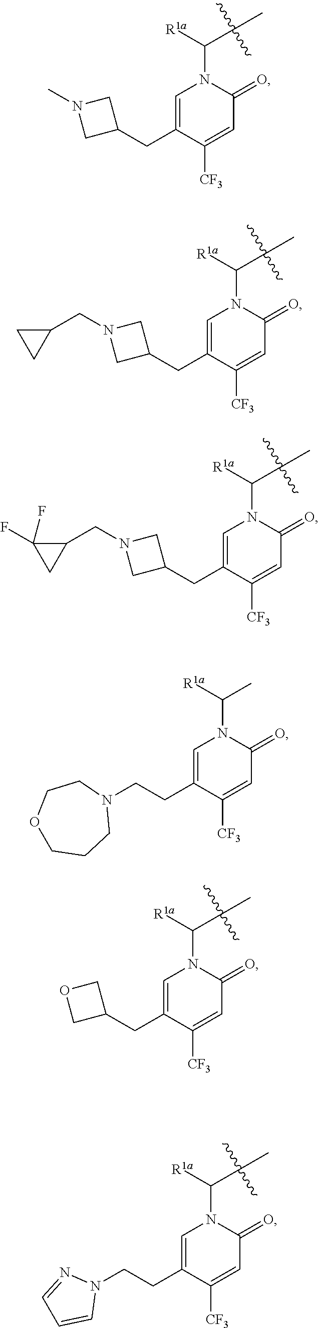

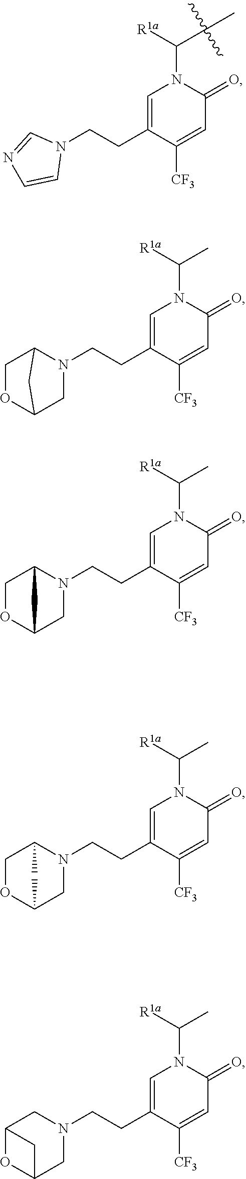

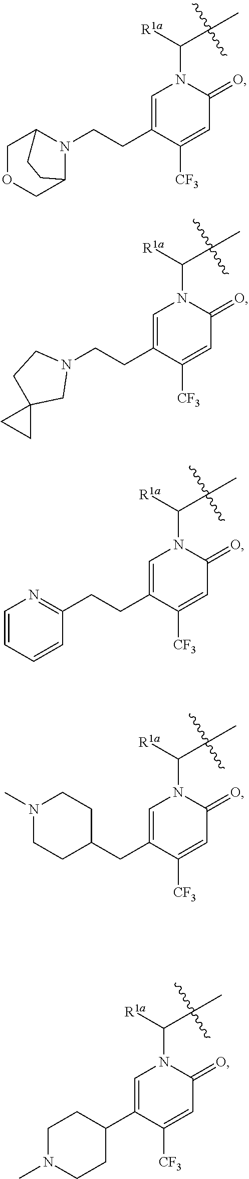

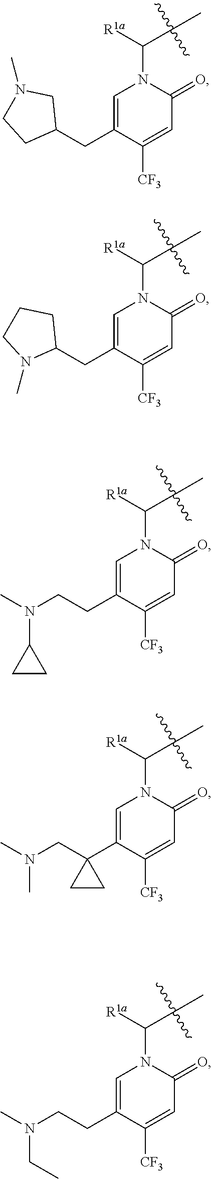

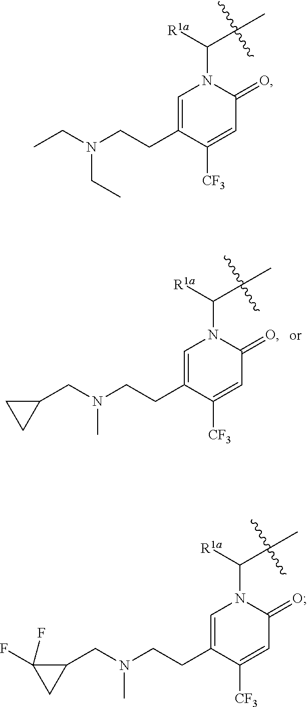

in some embodiments, R¹ is selected from

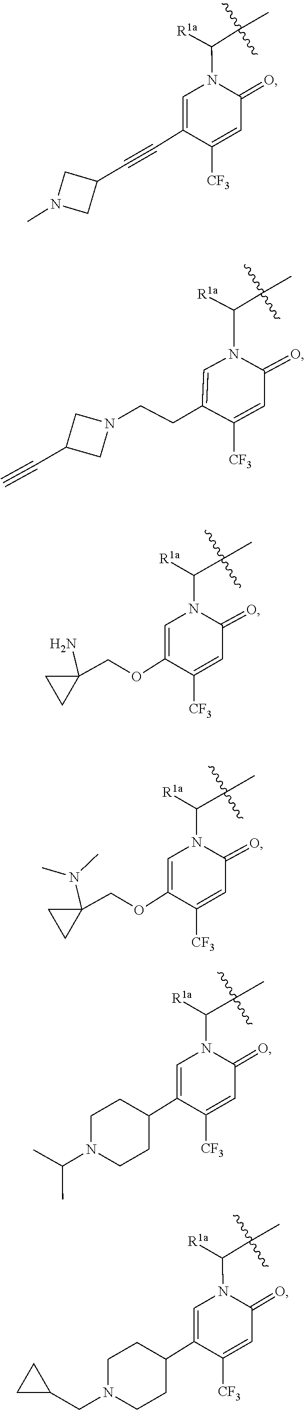

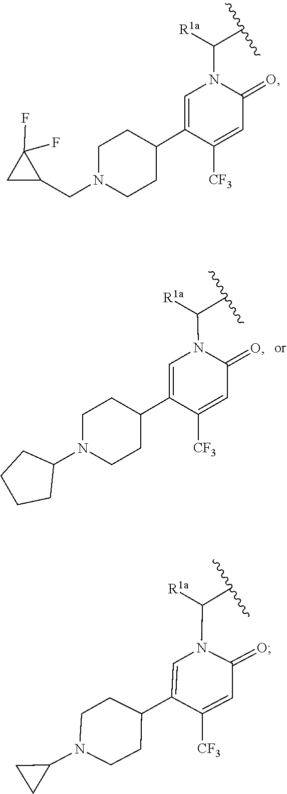

in some embodiments, R¹ is selected from

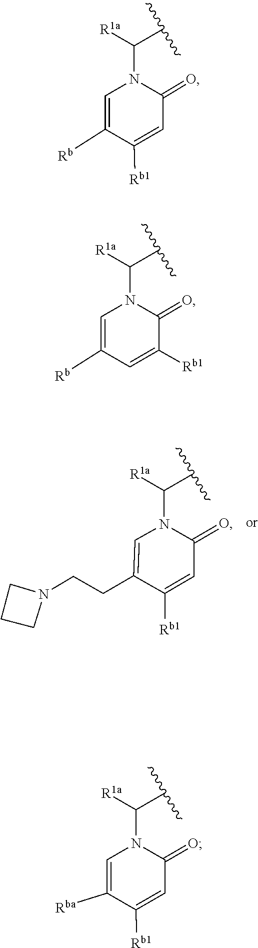

in some embodiments, R^{b1} is selected from H, C₁₋₄ alkyl, or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl, or C₁₋₄ alkoxy;
in some embodiments, R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, R^{bb} is selected from OH, cyano, C₁₋₄ alkyl, or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl or C₁₋₄ alkoxy;
in some embodiments, R^{1a} is selected from C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
in some embodiments, R^{1a} is selected from methyl, ethyl, propyl, butyl, isobutyl, sec-butyl, tert-butyl, —CH₂-cyclopropyl or —CH₂-cyclobutyl;
in some embodiments, R^{1a} is selected from

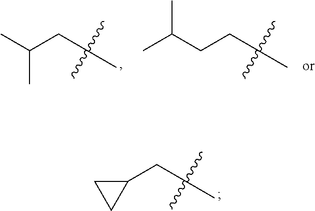

in some embodiments, R^{1b} is selected from a C₄₋₁₀ carbocycle or a 5- to 10-membered heterocycle, wherein the carbocycle or heterocycle is optionally substituted with 0 to 4 R^{b}, and the heterocycle contains 1 to 4 heteroatoms selected from O, S or N;
in some embodiments, each R^{b} is independently selected from H, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclyl, —C₀₋₄ alkyl-3- to 10-membered heterocyclyl or R^{ba}, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocycle contains 1 to 4 heteroatoms selected from O, S or N;
in some embodiments, each R^{b} is independently selected from H, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₁₋₄ alkoxy, —C₀₋₄ alkyl-C₃₋₆ carbocyclyl, —C₀₋₄ alkyl-3- to 7-membered heterocyclyl or R^{ba}, wherein the alkyl, alkoxy, carbocyclyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocycle contains 1 to 4 heteroatoms selected from O, S or N;
in some embodiments, R^{b} is selected from R^{ba};
in some embodiments, each R^{b} is independently selected from H, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, C₃₋₆ cycloalkyl, 3- to 7-membered heterocycloalkyl, —CH₂—C₃₋₆ cycloalkyl, —CH₂-3- to 7-membered heterocycloalkyl, —CH₂CH₂—C₃₋₆ cycloalkyl, —CH₂CH₂-3- to 7-membered heterocycloalkyl or R^{ba}, wherein the CH₂, alkyl, alkynyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, each R^{b} is independently selected from H, F, Cl, Br, I, OH, ═O, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azacyclopentyl, —CH₂-azacyclohexyl, —CH₂-oxetanyl, —CH₂-oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 0 to 4 substituents selected from H, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
in some embodiments, each R^{b} is independently selected from H, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂— cyclobutyl, —CH₂-cyclopentyl, —CH₂-cyclohexyl, —CH₂-azetidinyl, —CH₂-azacyclopentyl, —CH₂— azacyclohexyl, —CH₂-oxetanyl, —CH₂-oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 0 to 4 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, R^{ba} is selected from —C₀₋₄ alkyl-7- to 12-membered heterocycle, —C₀₋₄ alkyl-4- to 6-membered heterocycle connected via a carbon atom,

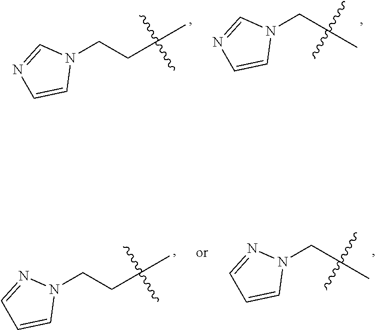 [SMILES: *CCn1ccnc1 ; *Cn1ccnc1 ; *CCn1cccn1 ; *Cn1cccn1]

and the R^{ba} is optionally substituted with 1 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocycle contains 1 to 4 heteroatoms selected from O, S or N;
in some embodiments, R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl,

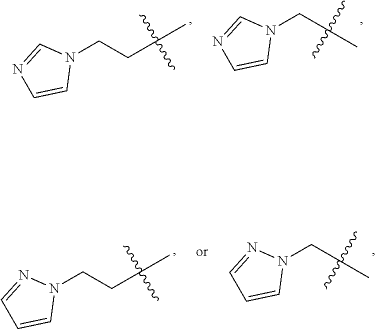 [SMILES: *CCn1ccnc1 ; *Cn1ccnc1 ; *CCn1cccn1 ; *Cn1cccn1]

and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
in some embodiments, R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom,

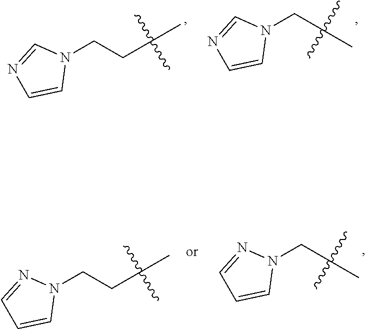 [SMILES: *CCn1ccnc1 ; *Cn1ccnc1 ; *CCn1cccn1 ; *Cn1cccn1]

and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
in some embodiments, R^{ba} is selected from one of the following optionally substituted groups:

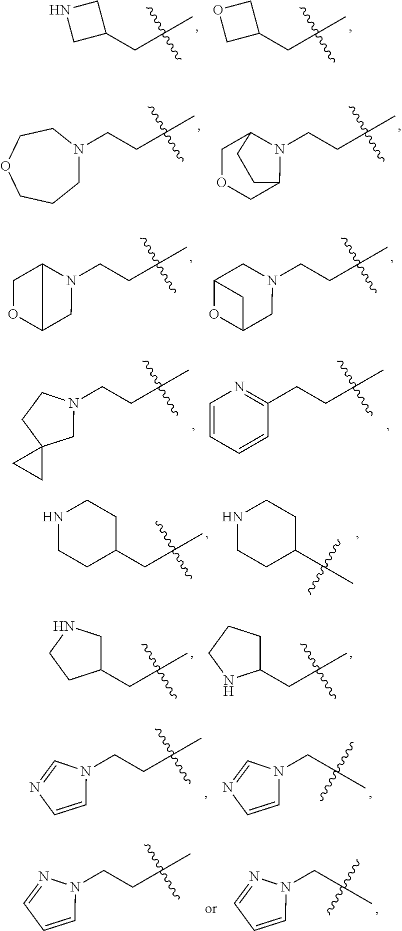

which, when substituted, is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}; in some embodiments, R^{ba} is selected from one of the following optionally substituted groups:

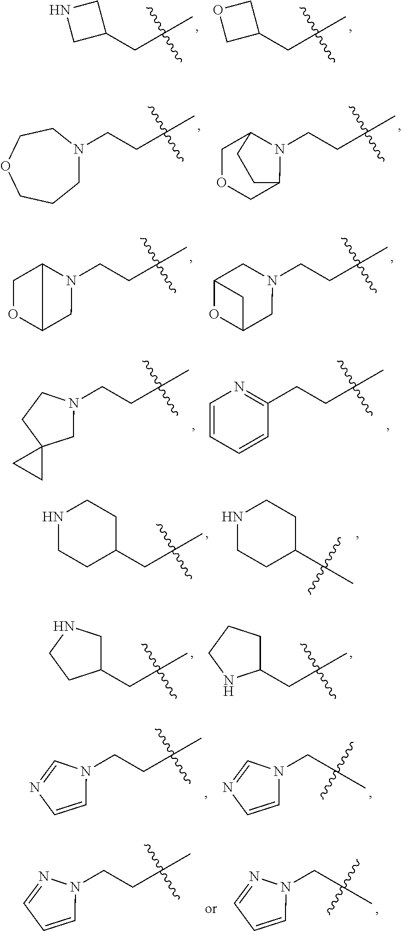

which, when substituted, is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclyl or —C₀₋₄ alkyl-3- to 10-membered heterocyclyl, wherein the alkyl, carbocyclyl or heterocyclyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclyl contains 1 to 4 heteroatoms selected from O, S or N;
in some embodiments, R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclyl or —C₀₋₂ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
in some embodiments, R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclyl or —C₀₋₄ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
in some embodiments, R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄ alkoxy or C₁₋₄ alkoxyalkyl;
in some embodiments, R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, methoxy, ethoxy, methoxymethyl, ethoxymethyl or methoxyethyl;
in some embodiments, R² is selected from C₁₋₆ alkyl, a C₆₋₁₀ aromatic ring group, a 5- to 10-membered heteroaromatic ring group, C₃₋₁₀ carbocyclyl or 5- to 10-membered heterocyclyl, and the R² is optionally substituted with 0 to 4 R^{2a}, wherein the heteroaromatic ring group and heterocyclyl contain 1 to 4 heteroatoms selected from O, S or N;
in some embodiments, R² is selected from C₁₋₄ alkyl, a benzene ring group, a naphthalene ring group, a 5- to 6-membered heteroaromatic ring group, a 9- to 10-membered heteroaromatic ring group, C₃₋₁₀ non-aromatic carbocyclyl or 5- to 10-membered non-aromatic heterocyclyl, and the R² is optionally substituted with 0 to 4 R^{2a}, wherein the heteroaromatic ring group and heterocyclyl contain 1 to 4 heteroatoms selected from O, S or N;
in some embodiments, R² is selected from a benzene ring group, a naphthalene ring group, a 5- to 6-membered heteroaromatic ring group, a 9- to 10-membered heteroaromatic ring group, C₃₋₆ cycloalkyl or 3- to 7-membered heterocycloalkyl, and the R² is optionally substituted with 0 to 4 R^{2a}, wherein the heteroaromatic ring group and heterocycloalkyl contain 1 to 4 heteroatoms selected from O, S or N;
in some embodiments, R² is selected from a benzene ring group, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl or

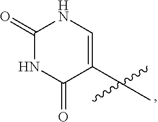 [SMILES: *c1c[nH]c(=O)[nH]c1=O]

and the R² is optionally substituted with 0 to 4 R^{2a};
in some embodiments, R² is selected from

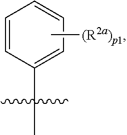

wherein p1 is selected from 0, 1, 2, 3 or 4;
in some embodiments, R^{2a}, is selected from H, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from halogen, OH, CN, C₁₋₄ alkyl, or C₁₋₄ alkoxy, and the heterocycloalkyl contains 1 to 4 heteroatoms selected from O, S or N;
in some embodiments, R^{2a}, is selected from F, Cl, Br, I, OH, cyano, methyl, ethyl, methoxy or ethoxy;
in some embodiments, R² is selected from phenyl, pyridyl, pyridone, azacyclopentyl, morpholinyl or

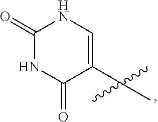 [SMILES: *c1c[nH]c(=O)[nH]c1=O]

and the R² is optionally substituted with 0 to 4 substituents selected from H, F, Cl, Br, OH, CF₃, cyano, methyl, ethyl, methoxy, ethoxy, cyclopropyl or cyclobutyl;
in some embodiments, R² is selected from

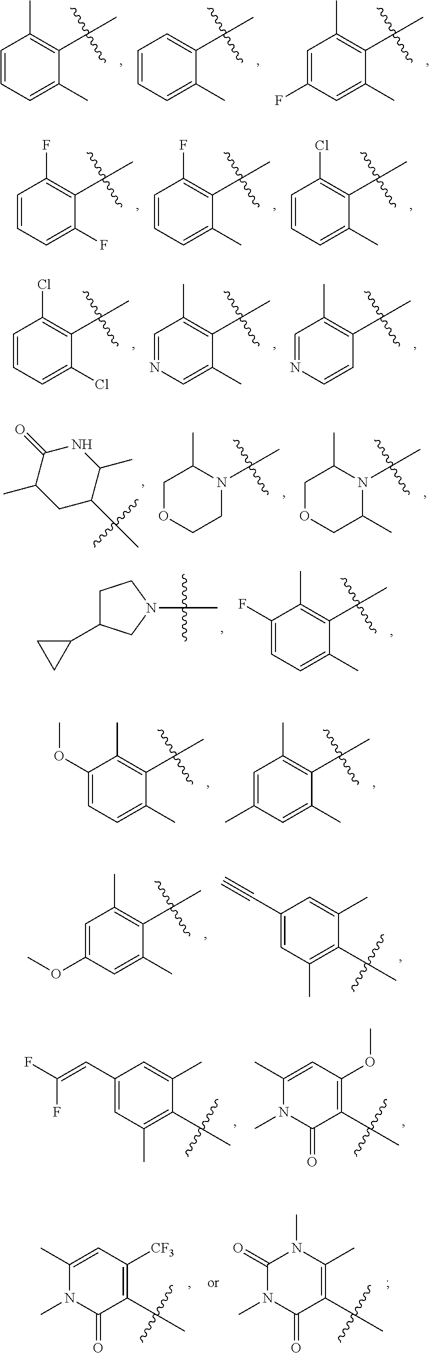

in some embodiments, R² is selected from

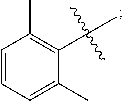 [SMILES: *c1c(C)cccc1C]

in some embodiments, each R^{2a}, is independently selected from H, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, each R^{2a}, is independently selected from H, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, each R^{2a}, is independently selected from H, halogen, OH, cyano, ═O, C₁₋₄ alkyl, C₁₋₄ alkoxy or C₃₋₆ cycloalkyl, wherein the alkyl, alkoxy or cycloalkyl is optionally substituted with 0 to 4 substituents selected from H, halogen, OH, ═O, cyano, C₁₋₄ alkyl or C₁₋₄ alkoxy;
in some embodiments, each R^{2a}, is independently selected from H, F, Cl, Br, I, OH, ═O, cyano, methyl, ethyl, methoxy, ethoxy, cyclopropyl or cyclobutyl, wherein the methyl, ethyl, methoxy, ethoxy, cyclopropyl or cyclobutyl is optionally substituted with 0 to 4 substituents selected from H, halogen, OH, ═O, cyano, C₁₋₄ alkyl or C₁₋₄ alkoxy;
in some embodiments, R³ is selected from H or C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ carbocyclyl;
in some embodiments, R³ is selected from H or C₁₋₄ alkyl, wherein the alkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclyl;
in some embodiments, R³ is selected from H or C₁₋₄ alkyl, wherein the alkyl is optionally substituted with 0 to 4 substituents selected from H, halogen, C₁₋₄ alkyl, C₁₋₄ alkoxy or a benzene ring group;
in some embodiments, R³ is selected from H, methyl, ethyl, propyl, butyl, isobutyl, sec-butyl, tert-butyl or benzyl;
in some embodiments, ring A is selected from a 5-membered heteroaromatic ring,

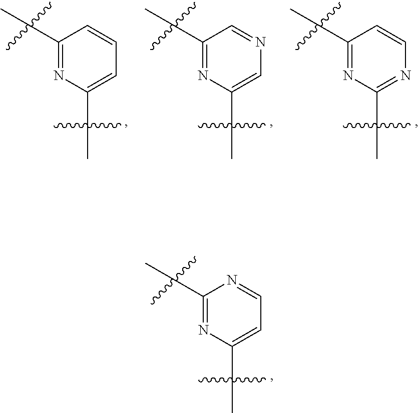 [SMILES: *c1cccc(*)n1 ; *c1cncc(*)n1 ; *c1ccnc(*)n1 ; *c1ccnc(*)n1]

or a 9- to 10-membered fused heteroaromatic ring, and the ring A is optionally substituted with 0 to 4 R^{a5}, wherein the heteroaromatic ring contains 1 to 4 heteroatoms selected from N, O or S;
in some embodiments, ring A is selected from a 5-membered heteroaromatic ring (such as thiophene, thiazole, furan, oxazole, pyrrole, pyrazole, or imidazole),

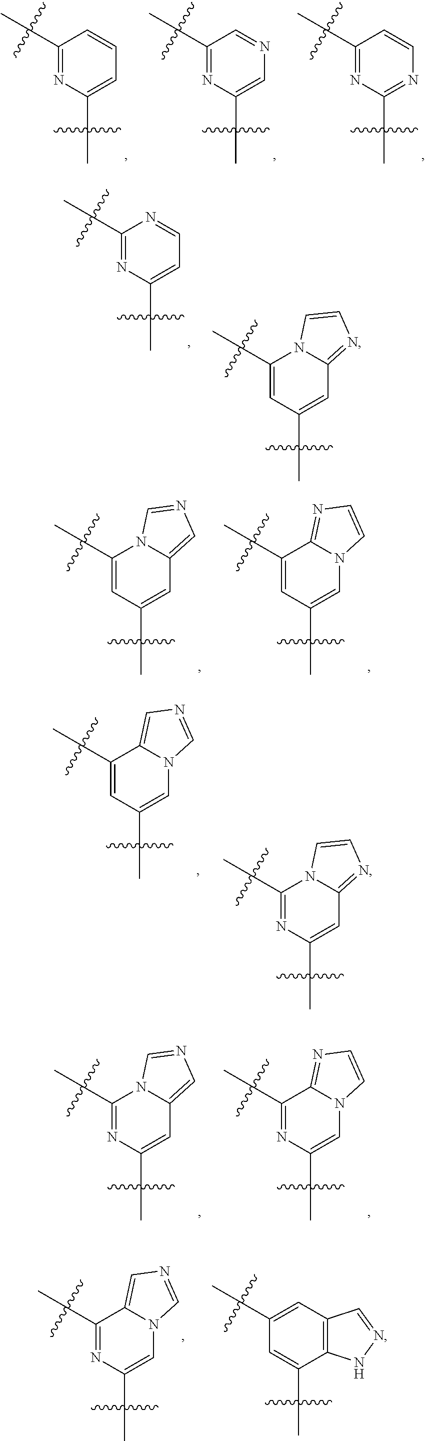

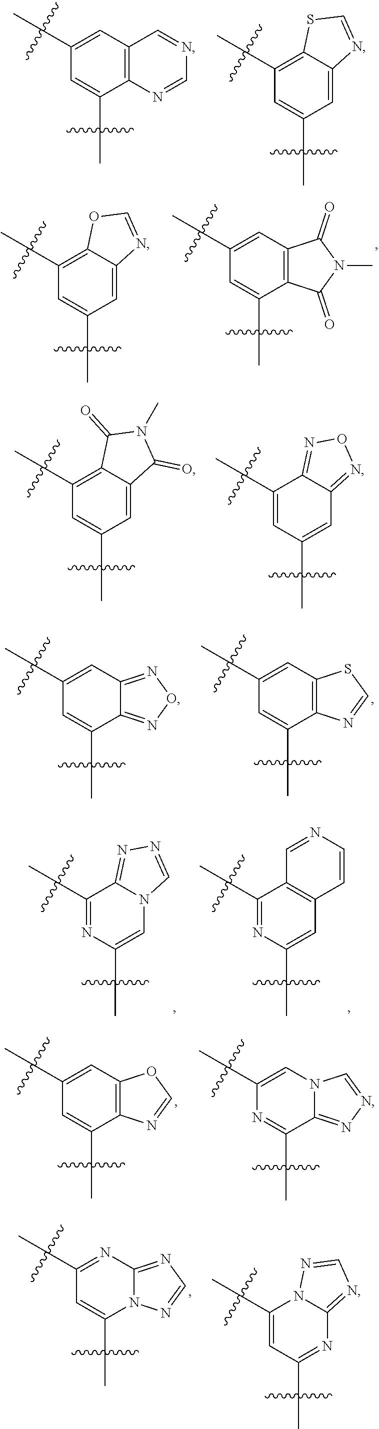

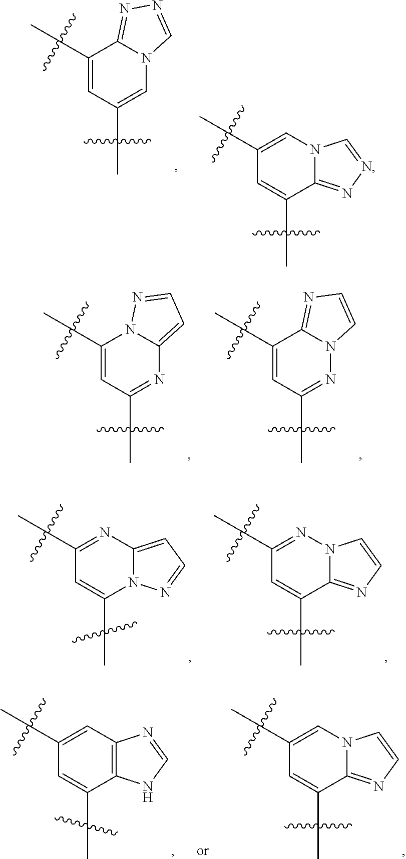

and the ring A is optionally further substituted with 0 to 4 R^{a5}, wherein the heteroaromatic ring contains 1 to 2 heteroatoms selected from N, O or S;
in some embodiments, ring A is selected from

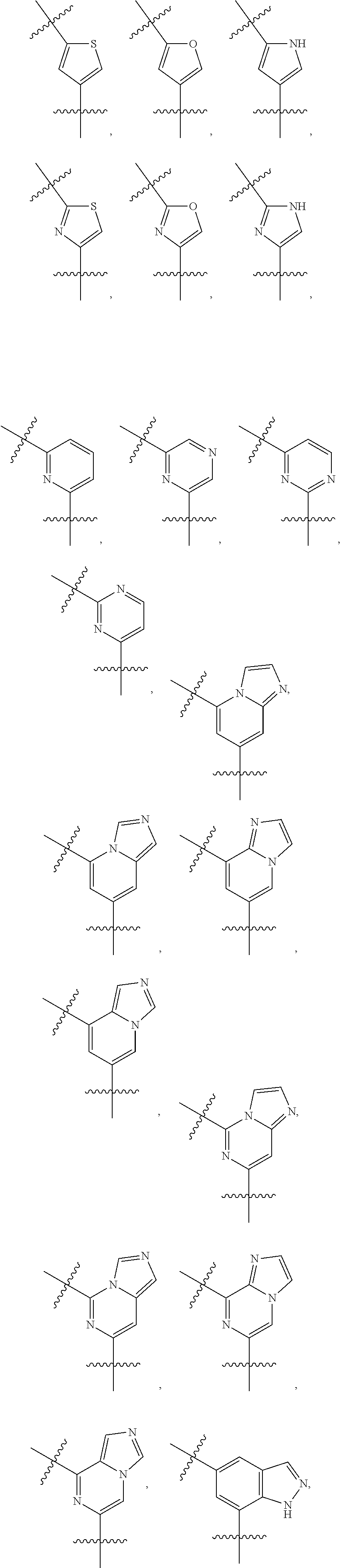

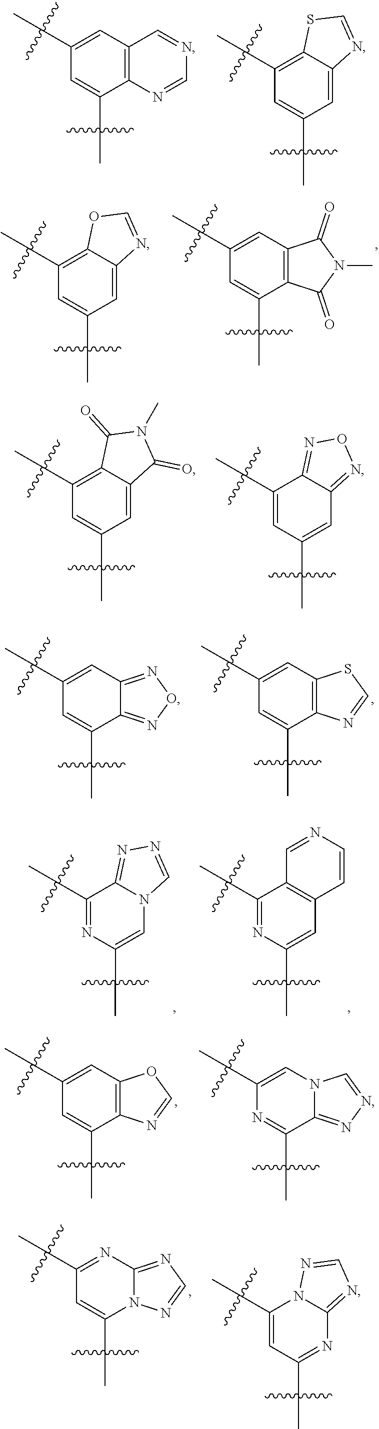

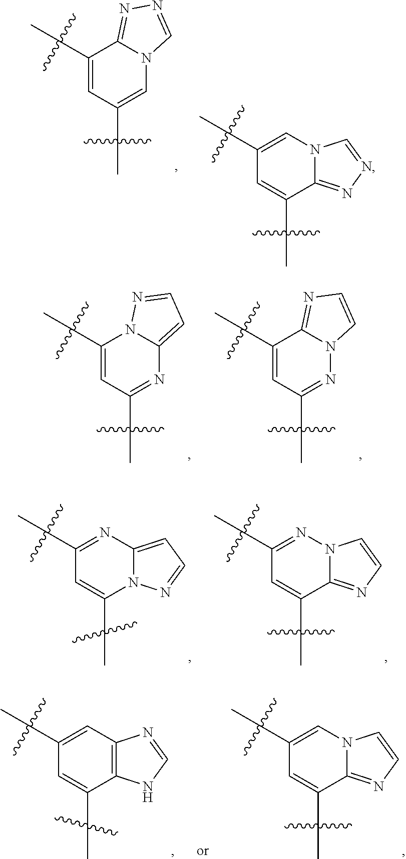

and the ring A is optionally substituted with 0 to 4 R^{a5};
in some embodiments, ring A is selected from

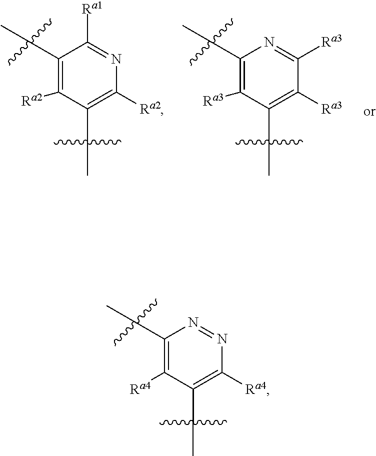

the upper left part of which is directly connected to R²;
in some embodiments, R^{a1} is selected from H, halogen, OH, cyano, NH₂, NHC₁₋₆ alkyl, N(C₁₋₆ alkyl)₂, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, cycloalkyl or heterocyclyl is substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and at least one of the substituents is not halogen, the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, R^{a1} is selected from H, halogen, OH, cyano, NH₂, NHC₁₋₄ alkyl, N(C₁₋₄ alkyl)₂, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, R^{a1} is selected from H, halogen, OH, cyano, C₂₋₄ alkenyl, C₂₋₄ alkynyl or C₁₋₄ alkoxy, wherein the alkenyl, alkynyl, or alkoxy is optionally substituted with 0 to 4 substituents selected from halogen, OH, ═O, cyano, C₁₋₄ alkyl or C₁₋₄ alkoxy;
in some embodiments, R^{a1} is selected from H, F, Cl, Br, I, OH, cyano, ethenyl, ethynyl, methoxy or ethoxy, wherein the ethenyl, ethynyl, methoxy or ethoxy is optionally substituted with 0 to 4 substituents selected from halogen, OH, ═O, cyano, C₁₋₄ alkyl or C₁₋₄ alkoxy; in some embodiments, R^{a1} is selected from H, F, Cl, Br, I, OH, cyano, ethenyl, ethynyl, methoxy, —OCF₃ or ethoxy;
in some embodiments, each R^{a2} is independently selected from H, halogen, OH, cyano, NH₂, NHC₁₋₆ alkyl, N(C₁₋₆ alkyl)₂, C₁₋₆ alkyl, C₁₋₆ alkoxy, C₂₋₆ alkenyl, C₂₋₆ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, each R^{a2} is independently selected from H, halogen, OH, cyano, NH₂, NHC₁₋₄ alkyl, N(C₁₋₄ alkyl)₂, C₁₋₄ alkyl, C₁₋₄ alkoxy, C₂₋₄ alkenyl, C₂₋₄ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, each R^{a2} is independently selected from H, halogen, OH, cyano, C₁₋₄ alkyl or C₁₋₄ alkoxy, wherein the alkyl or alkoxy is optionally substituted with 0 to 4 substituents selected from halogen, OH, ═O, cyano, C₁₋₄ alkyl or C₁₋₄ alkoxy;
in some embodiments, each R^{a2} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from halogen, OH, ═O, cyano, C₁₋₄ alkyl or C₁₋₄ alkoxy;
in some embodiments, each R^{a2} is independently selected from H, F, Cl, Br, I, OH, cyano, methyl, ethyl, methoxy, —OCF₃ or ethoxy;
in some embodiments, each R^{a3} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, each R^{a3} is independently selected from H, halogen, OH, cyano, C₁₋₄ alkyl, C₂₋₄ alkenyl, C₂₋₄ alkynyl, C₁₋₄ alkoxy, C₃₋₄ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, each R^{a3} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy;
in some embodiments, each R^{a3} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
in some embodiments, each R^{a3} is independently selected from H, F, Cl, Br, I, OH, cyano, methyl, ethyl, methoxy, —OCF₃ or ethoxy;
in some embodiments, each R^{a4} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, each R^{a4} is independently selected from H, halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, each R^{a4} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy;
in some embodiments, each R^{a4} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy; in some embodiments, each R^{a4} is independently selected from H, F, Cl, Br, I, OH, cyano, methyl, ethyl, methoxy, —OCF₃ or ethoxy;
in some embodiments, the compound optionally meets (one of) the following conditions: 1) when R^{a1} is selected from H, unsubstituted C₁₋₆ alkyl, unsubstituted C₃₋₆ cycloalkyl or unsubstituted 3- to 7-membered heterocyclyl, R¹ is selected from —NR^{1a}R^{1b}, or at least one R^{a2} is not H;
2) when R¹ is selected from —CHR^{1a}R^{1b}, at least one R^{a3} is not H;
3) when R¹ is selected from —CHR^{1a}R^{1b}, at least one R^{a4} is not H;
4) the compound is not

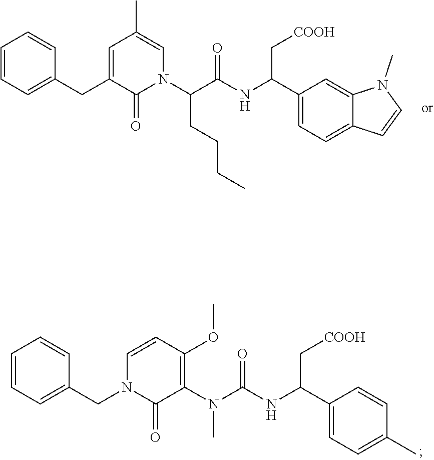 [SMILES: CCCCC(C(=O)NC(CC(=O)O)c1ccc2ccn(C)c2c1)n1cc(C)cc(Cc2ccccc2)c1=O ; COc1ccn(Cc2ccccc2)c(=O)c1N(C)C(=O)NC(CC(=O)O)c1ccc(C)cc1]

in some embodiments, the compound optionally meets the following conditions:
1) when R^{a1} is selected from H, unsubstituted C₁₋₄ alkyl, unsubstituted C₃₋₆ cycloalkyl, or unsubstituted 3- to 7-membered heterocyclyl, R¹ is selected from

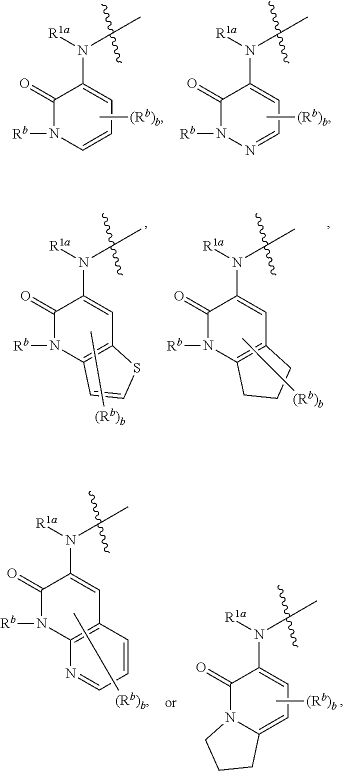

or at least one R^{a2} is not H;
2) when R¹ is selected from

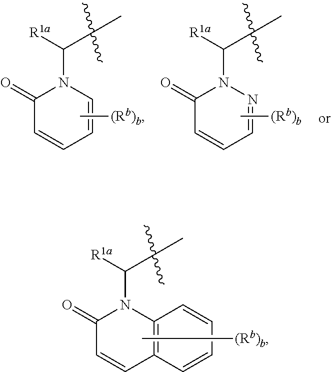

at least one R^{a3} is not H;
3) when R¹ is selected from

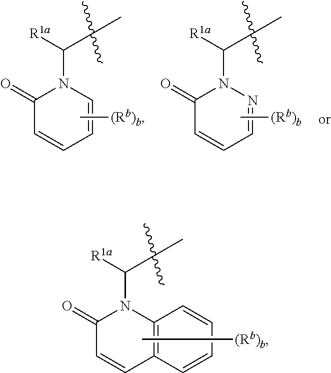

at least one R^{a4} is not H;
in some embodiments, the compound optionally meets the following conditions:
1) when R^{a1} is selected from H, R¹ is selected from

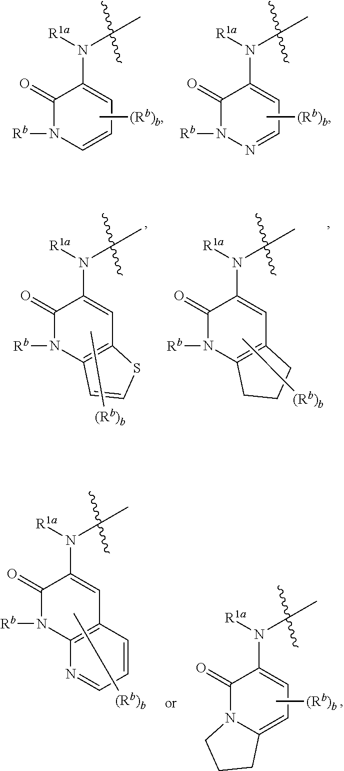

or at least one R^{a2} is not H;
2) when R¹ is selected from

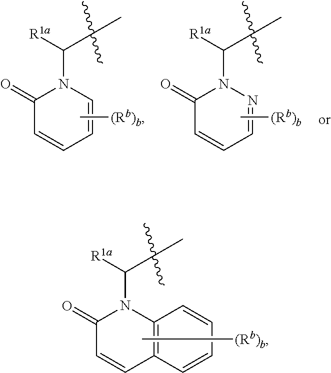

at least one R^{a3} is not H;
3) when R¹ is selected from

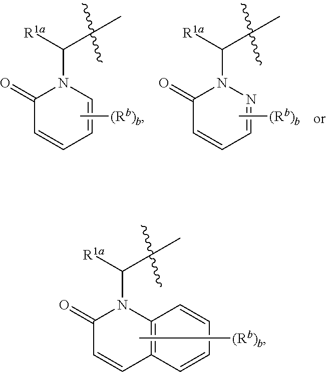

at least one R^{a4} is not H;
in some embodiments, ring A is selected from

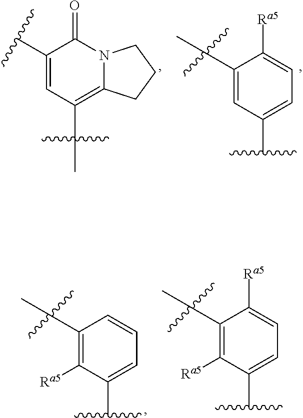

or a C₈₋₁₀ fused carbocycle (such as a naphthalene ring, or a benzo C₄₋₆ carbocycle), and the ring A is optionally substituted with 0 to 4 R^{a5};
in some embodiments, ring A is selected from

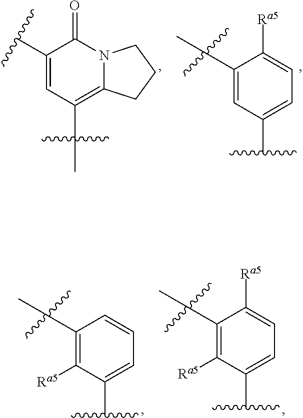

a naphthalene ring

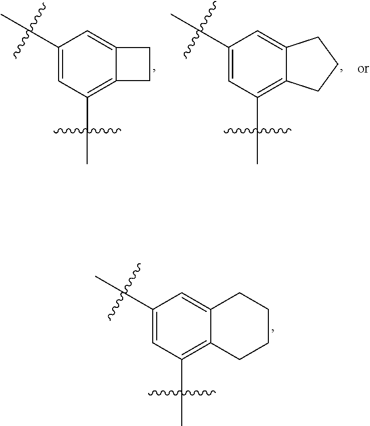 [SMILES: *c1cc(*)c2c(c1)CC2 ; *c1cc(*)c2c(c1)CCC2 ; *c1cc(*)c2c(c1)CCCC2]

and the ring A is optionally substituted with 0 to 4 R^{a5};
in some embodiments, ring A is selected from

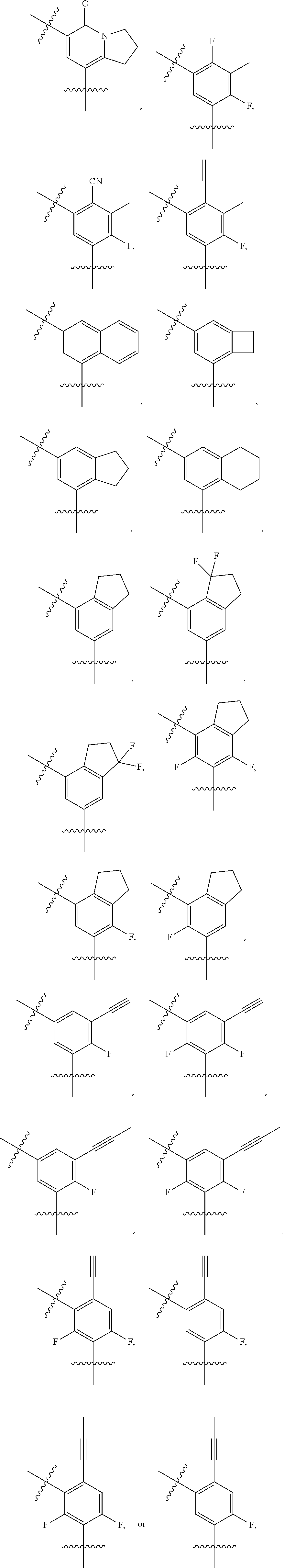

in some embodiments, ring A is selected from

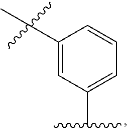 [SMILES: *c1cccc(*)c1]

and the ring A is substituted with one substituent selected from C₂₋₆ alkenyl or C₂₋₆ alkynyl and optionally substituted with 1 to 3 substituents selected from R^{a5};
in some embodiments, ring A is selected from

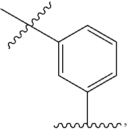 [SMILES: *c1cccc(*)c1]

and the ring A is substituted with one R^{ak} and optionally substituted with 1 to 3 substituents selected from R^{a5};
in some embodiments, ring A is selected from

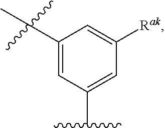

and the ring A is optionally substituted with 1 to 3 substituents selected from R^{a5};
in some embodiments, R^{ak} is selected from C₂₋₄ alkynyl (such as ethynyl, propynyl or propargyl);
in some embodiments, ring A is selected from

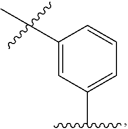 [SMILES: *c1cccc(*)c1]

and the ring A is substituted with one substituent selected from ethenyl, ethynyl, propynyl or propargyl and optionally substituted with 1 to 3 substituents selected from R^{a5};
in some embodiments, R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S;
in some embodiments, R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
in some embodiments, R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from halogen, OH, ═O, cyano, C₁₋₄ alkyl, C₁₋₄ alkoxy or C₃₋₆ cycloalkyl;
in some embodiments, each R^{a5} is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from halogen, OH, ═O, cyano, C₁₋₄ alkyl, C₁₋₄ alkoxy or C₃₋₆ cycloalkyl;
in some embodiments, each R^{a5} is independently selected from F, Cl, Br, I, OH, CN, ethynyl, methyl, or ethyl;
in some embodiments, each R^{a5} is independently selected from H, halogen, OH, ═O, cyano, COOH, NH₂, NHC₁₋₆ alkyl, N(C₁₋₆ alkyl)₂, C₁₋₆ alkyl, halogen-substituted C₁₋₆ alkyl, cyano-substituted C₁₋₆ alkyl, C₁₋₆ alkoxy or C₃₋₆ cycloalkyl;
in some embodiments, each R^{a5} is independently selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl;
in some embodiments, each R^{a5} is independently selected from F, Cl, Br, OH, cyano, CF₃, methyl, ethyl, methoxy, ethoxy, cyclopropyl or cyclobutyl;
in some embodiments, R^{1b} is selected from

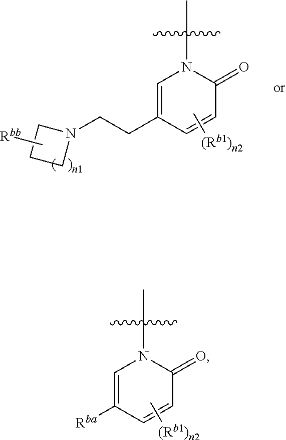

and ring A is selected from

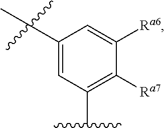

the upper left part of which is directly connected to R², wherein n1 is selected from 1, 2, 3 or 4, and n2 is selected from 0, 1, 2, 3 or 4;
in some embodiments, when ring A is selected from

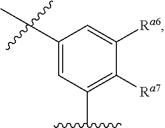

the upper left part of which is directly connected to R², R² is selected from a 5- to 10-membered heteroaromatic ring or a C₆₋₁₀ aromatic ring, wherein the aromatic ring is substituted with one R^{2aa} and optionally substituted with 1 to 3 R^{2a}, and the heteroaromatic ring is optionally substituted with 1 to 4 R^{2a}, and contains 1 to 4 heteroatoms selected from O, S or N;
in some embodiments, ring A is selected from

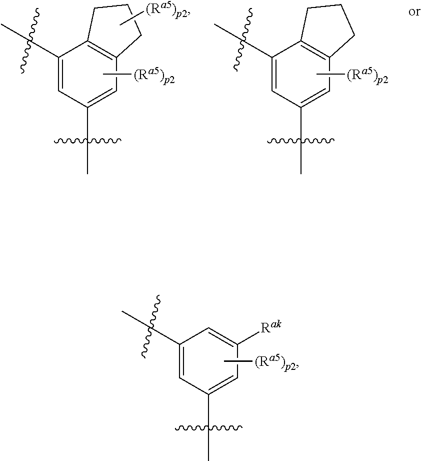

the upper left part of which is directly connected to R², wherein p2 is selected from 0, 1 or 2;
in some embodiments, R^{2aa} is selected from C₂₋₆ alkenyl or C₂₋₆ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
in some embodiments, R^{2aa} is selected from C₂₋₄ alkenyl or C₂₋₄ alkynyl, wherein the alkenyl or alkynyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy;
in some embodiments, R^{2aa} is selected from C₂₋₄ alkenyl or C₂₋₄ alkynyl, wherein the alkenyl or alkynyl is optionally substituted with 1 to 4 substituents selected from F, Cl, Br, I, OH, cyano, methyl, ethyl, methoxy or ethoxy;
in some embodiments, R^{1b} is selected from

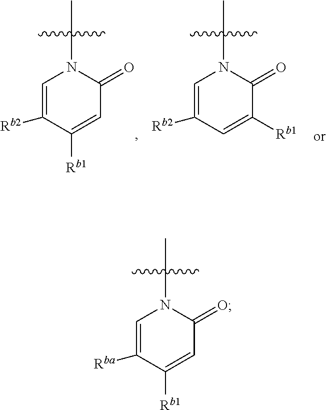

in some embodiments, R^{1b} is selected from

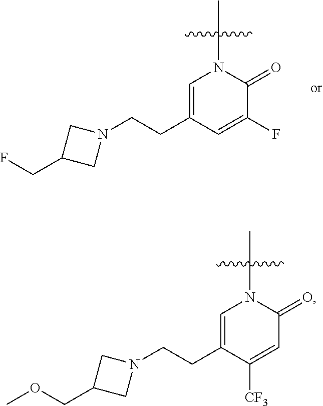 [SMILES: *n1cc(CCN2CC(CF)C2)cc(F)c1=O ; *n1cc(CCN2CC(COC)C2)c(C(F)(F)F)cc1=O]

and ring A is selected from

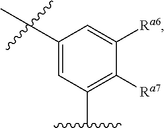

the upper left part of which is directly connected to R²;
in some embodiments, R^{1b} is selected from

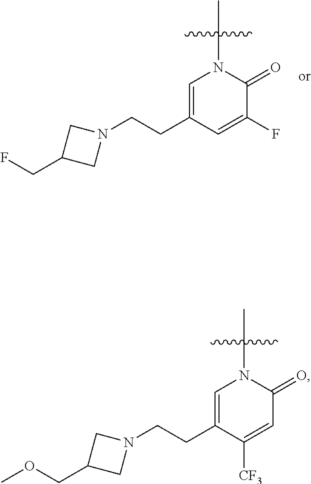 [SMILES: *n1cc(CCN2CC(CF)C2)cc(F)c1=O ; *n1cc(CCN2CC(COC)C2)c(C(F)(F)F)cc1=O]

and ring A is selected from

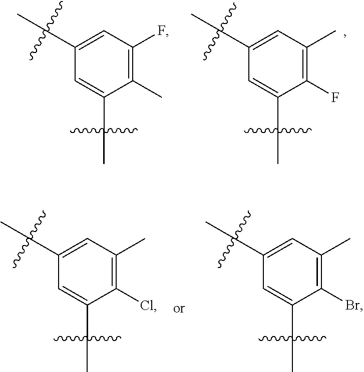

the upper left part of which is directly connected to R²;
in some embodiments, each R^{b1} is independently selected from H, halogen, CN, C₁₋₆ alkyl, or C₃₋₆ cycloalkyl, wherein the alkyl or cycloalkyl is optionally substituted with 0 to 4 substituents selected from halogen, OH, CN, NHC₁₋₆ alkyl, N(C₁₋₆ alkyl)₂, C₁₋₄ alkyl or C₁₋₆ alkoxy;
in some embodiments, each R^{b1} is independently selected from H, halogen, CN, C₁₋₄ alkyl, or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
in some embodiments, R^{bb} is selected from OH, cyano, C₁₋₆ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy;
in some embodiments, R^{bb} is selected from halogen, OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, or C₁₋₄ alkoxy;
in some embodiments, R^{a6} and R^{a7} are each independently selected from halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, R^{a6} and R^{a7} are each independently selected from F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
in some embodiments, the compound as represented by general formula (I) is selected from a compound as represented by general formula (I-a), (I-b), (I-c), (I-d) or (I-e),

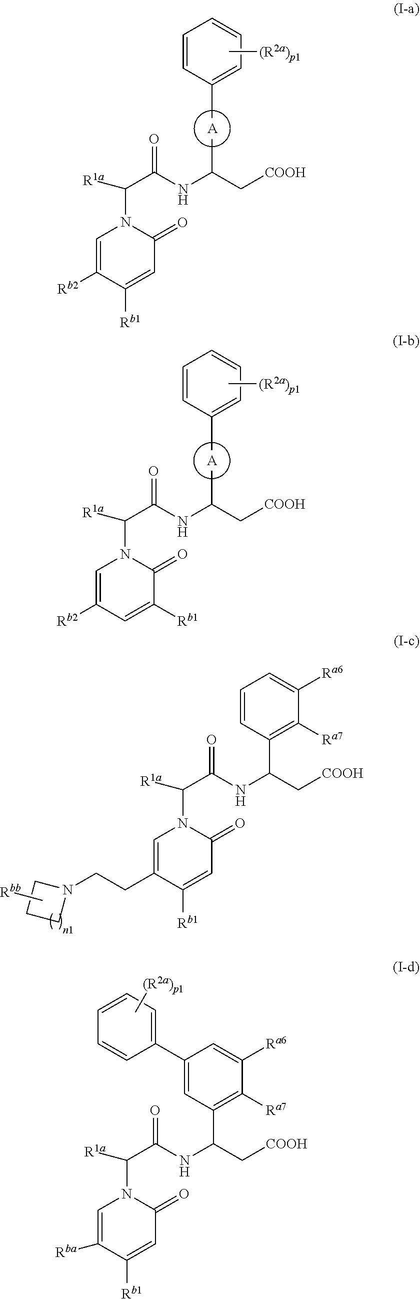

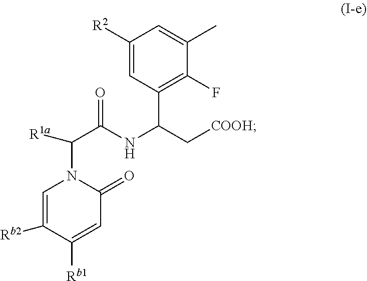

in some embodiments, the compound as represented by general formula (I) is selected from a compound as represented by general formula (I-f) or (I-g),

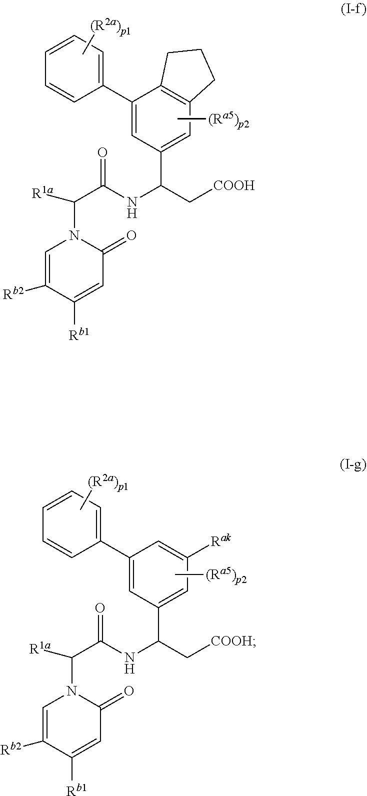

in some embodiments, in general formula (I-a), (I-b), (I-c), (I-d), (I-e), (I-f) or (I-g), the definitions of R^{2a}, R^{1a}, R^{bb}, R^{a6} and R^{a7} are the same as those in any one of the preceding embodiments;
in some embodiments, in general formula (I-a) or (I-b), the definition of ring A is the same as that in any one of the preceding embodiments of ring A;
in some embodiments, in general formula (I), (I-a), (I-b), (I-c), (I-d), (I-f) or (I-g), p1 is selected from 0, 1, 2, 3 or 4;
in some embodiments, in general formula (I), (I-a), (I-b), (I-c), (I-d), (I-f) or (I-g), R^{2a} is selected from H, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from halogen, OH, CN, C₁₋₄ alkyl, or C₁₋₄ alkoxy, and the heterocycloalkyl contains 1 to 4 heteroatoms selected from O, S or N;
in some embodiments, in general formula (I), (I-a), (I-b), (I-c), (I-d), (I-e), (I-f) or (I-g), R^{1a} is selected from

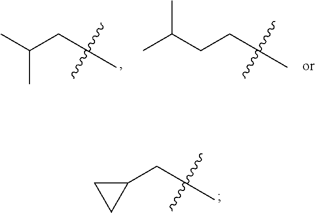

in some embodiments, in general formula (I), (I-a), (I-b), (I-c), (I-d), (I-e), (I-f) or (I-g), R^{b1} is selected from H, C₁₋₄ alkyl, or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl, or C₁₋₄ alkoxy;
in some embodiments, in general formula (I), (I-a), (I-b), (I-c), (I-d), (I-e), (I-f) or (I-g), R^{b1} is selected from H, F, CH₂F, CHF₂, CF₃, methyl, or —CH₂CH₂N(CH₃)₂;
in some embodiments, in general formula (I), (I-a), (I-b), (I-c), (I-e), (I-f) or (I-g), R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, in general formula (I), (I-a), (I-b), (I-c), (I-e), (I-f) or (I-g), R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 1, 2 or 3 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, in general formula (I-a), (I-b), (I-c), (I-d), (I-e), (I-f) or (I-g), the definitions of R^{ba} and R^{k} are the same as those in any one of the preceding embodiments; in some embodiments, in general formula (I), (I-a), (I-b), (I-c), (I-d), (I-f) or (I-g), R^{2a} is selected from F, Cl, Br, I, OH, cyano, methyl, ethyl, methoxy or ethoxy;
in some embodiments, in general formula (I), (I-f) or (I-g), p2 is selected from 0, 1 or 2;
in some embodiments, in general formula (I) or (I-g), R^{ak} is selected from C₂₋₄ alkynyl;
in some embodiments, in general formula (I), (I-f) or (I-g), R^{a5} is selected from F, Cl, Br, I, OH, cyano, methyl, ethyl, methoxy, ethoxy or ethynyl;
in some embodiments, in general formula (I-e), R² is selected from a 5- to 6-membered heteroaromatic ring group or phenyl, wherein the phenyl is substituted with one R^{2aa} and optionally substituted with 1 to 3 R^{2a}, the heteroaromatic ring is optionally substituted with 1 to 4 R^{2a} and contains 1 to 3 heteroatoms selected from O, S or N, and R^{2aa} is selected from C₂₋₄ alkenyl or C₂₋₄ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
in some embodiments, in general formula (I-e), R² is selected from

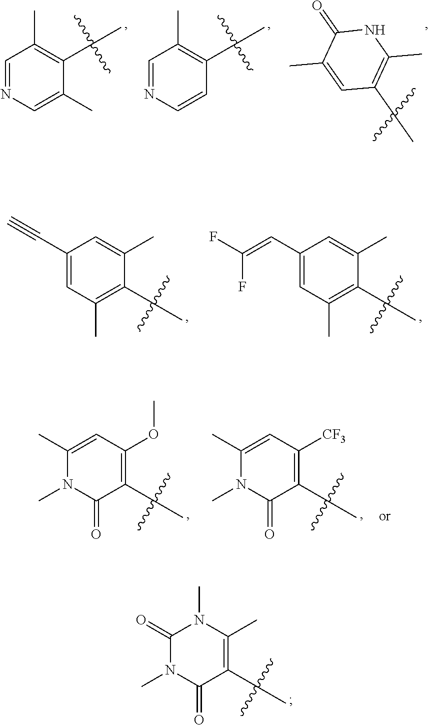

in some embodiments, in general formula (I), (I-c) or (I-d), R^{a6} and R^{a7} are each independently selected from F, Cl, Br, I, OH, cyano, methyl, ethyl, methoxy or ethoxy;
in some embodiments, in general formula (I) or (I-c), R^{bb} is selected from OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, or C₁₋₄ alkoxy;
in some embodiments, in general formula (I) or (I-c), R^{bb} is selected from CH₂F, CHF₂, CF₃, methyl, ethyl, or methoxymethyl; and
in some embodiments, in general formula (I) or (I-c), n1 is selected from 1, 2 or 3.

As a first embodiment of the present disclosure, provided is the compound as represented by the preceding general formula (I) or a stereoisomer, a deuterated form, a solvate, a prodrug, a metabolite, a pharmaceutically acceptable salt or a co-crystal thereof, wherein
R¹ is selected from —CHR^{1a}R^{1b} or —NR^{1a}R^{1b};
R^{1a} is selected from C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
R^{1b} is selected from C₄₋₁₀ carbocyclyl or 5- to 10-membered heterocyclyl, wherein the carbocyclyl or heterocyclyl is optionally substituted with 0 to 4 R^{b}, and the heterocyclyl contains 1 to 4 heteroatoms selected from O, S or N;
each R^{b} is independently selected from H, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclyl, —C₀₋₄ alkyl-3- to 10-membered heterocyclyl or R^{ba}, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocycle contains 1 to 4 heteroatoms selected from O, S or N;
R^{ba} is selected from —C₀₋₄ alkyl-7- to 12-membered heterocyclyl, —C₀₋₄ alkyl-4- to 6-membered heterocyclyl connected via a carbon atom,

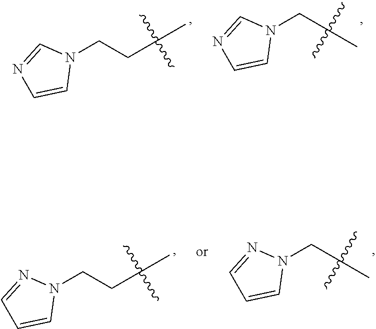 [SMILES: *CCn1ccnc1 ; *Cn1ccnc1 ; *CCn1cccn1 ; *Cn1cccn1]

and the R^{ba} is optionally substituted with 1 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocycle contains 1 to 4 heteroatoms selected from O, S or N;
R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclyl or —C₀₋₄ alkyl-3- to 10-membered heterocyclyl, wherein the alkyl, carbocyclyl or heterocyclyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, the heterocyclyl contains 1 to 4 heteroatoms selected from O, S or N, and the heterocycle contains 1 to 4 heteroatoms selected from O, S or N;
R² is selected from C₁₋₆ alkyl, a C₆₋₁₀ aromatic ring group, a 5- to 10-membered heteroaromatic ring group, C₃₋₁₀ carbocyclyl or 5- to 10-membered heterocyclyl, and the R² is optionally substituted with 0 to 4 R^{2a}, wherein the heteroaromatic ring group and heterocyclyl contain 1 to 4 heteroatoms selected from O, S or N;
each R^{2a}, is independently selected from H, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
R³ is selected from H or C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ carbocyclyl;
ring A is selected from a 5-membered heteroaromatic ring,

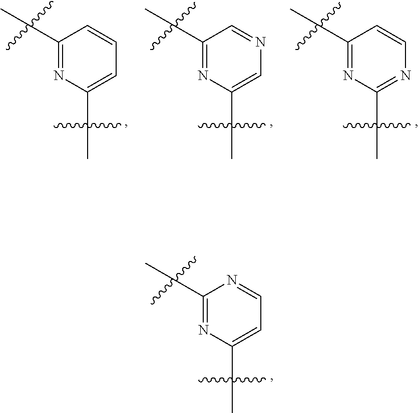 [SMILES: *c1cccc(*)n1 ; *c1cncc(*)n1 ; *c1ccnc(*)n1 ; *c1ccnc(*)n1]

or a 9- to 10-membered fused heteroaromatic ring, and the ring A is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, halogen-substituted C₁₋₆ alkyl, cyano-substituted C₁₋₆ alkyl, C₁₋₆ alkoxy or C₃₋₆ cycloalkyl, wherein the heteroaromatic ring contains 1 to 4 heteroatoms selected from N, O or S;
or ring A is selected from

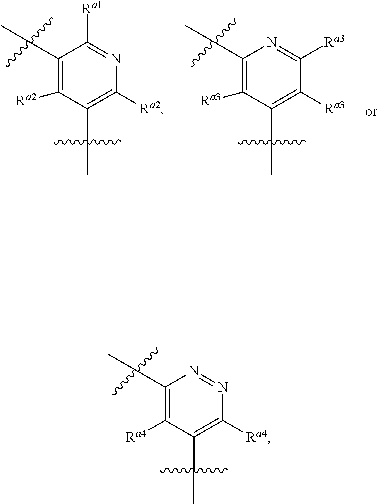

the upper left part of which is directly connected to R²;
R^{a1} is selected from H, halogen, OH, cyano, NH₂, NHC₁₋₆ alkyl, N(C₁₋₆ alkyl)₂, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, cycloalkyl or heterocyclyl is substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and at least one of the substituents is not halogen, the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a2} is independently selected from H, halogen, OH, cyano, NH₂, NHC₁₋₆ alkyl, N(C₁₋₆ alkyl)₂, C₁₋₆ alkyl, C₁₋₆ alkoxy, C₂₋₆ alkenyl, C₂₋₆ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a3} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a4} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S,
provided that 1) when R^{a1} is selected from H, unsubstituted C₁₋₆ alkyl, unsubstituted C₃₋₆ cycloalkyl or unsubstituted 3- to 7-membered heterocyclyl, R¹ is selected from —NR^{1a}R^{1b}, or at least one R^{a2} is not H;
2) when R¹ is selected from —CHR^{1a}R^{1b}, at least one R^{a3} is not H;
3) when R¹ is selected from —CHR^{1a}R^{1b}, at least one R^{a4} is not H;
4) the compound is not

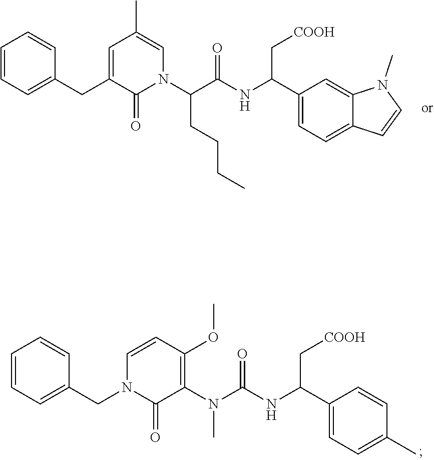 [SMILES: CCCCC(C(=O)NC(CC(=O)O)c1ccc2ccn(C)c2c1)n1cc(C)cc(Cc2ccccc2)c1=O ; COc1ccn(Cc2ccccc2)c(=O)c1N(C)C(=O)NC(CC(=O)O)c1ccc(C)cc1]

or ring A is selected from

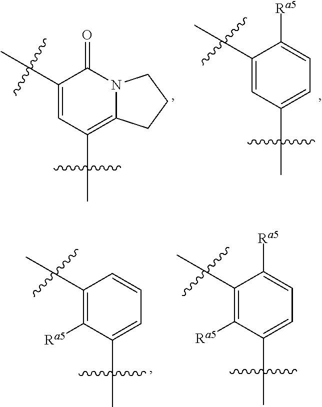

or a C₈₋₁₀ fused carbocycle, and the ring A is optionally substituted with 0 to 4 R^{a5};
or ring A is selected from

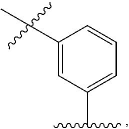 [SMILES: *c1cccc(*)c1]

and the ring A is substituted with one substituent selected from C₂₋₆ alkenyl or C₂₋₆ alkynyl and optionally substituted with 1 to 3 R^{a5};
R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S;
or when ring A is selected from

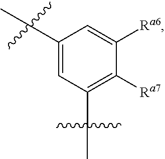

the upper left part of which is directly connected to R², one of the following conditions is met: 1) R^{1b} is selected from

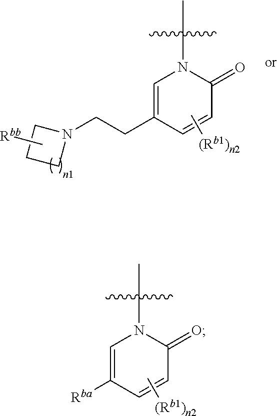

2) R² is selected from a 5- to 10-membered heteroaromatic ring group or a C₆₋₁₀ aromatic ring group, wherein the aromatic ring group is substituted with one R^{2aa} and optionally substituted with 1 to 3 R^{2a}, and the heteroaromatic ring group is optionally substituted with 1 to 4 R^{2a} and contains 1 to 4 heteroatoms selected from O, S or N;
R^{2aa} is selected from C₂₋₆ alkenyl or C₂₋₆ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
n1 is selected from 1, 2, 3 or 4;
n2 is selected from 0, 1, 2, 3 or 4;
each R^{b1} is independently selected from H, halogen, CN, C₁₋₆ alkyl, or C₃₋₆ cycloalkyl, wherein the alkyl or cycloalkyl is optionally substituted with 0 to 4 substituents selected from halogen, OH, CN, NHC₁₋₆ alkyl, N(C₁₋₆ alkyl)₂, C₁₋₄ alkyl or C₁₋₆ alkoxy;
R^{bb} is selected from OH, cyano, C₁₋₆ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy;
R^{a6} and R^{a7} are each independently selected from halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S.

As a second embodiment of the present disclosure, provided is the compound as represented by the preceding general formula (I) or a stereoisomer, a deuterated form, a solvate, a prodrug, a metabolite, a pharmaceutically acceptable salt or a co-crystal thereof, wherein
R¹ is selected from

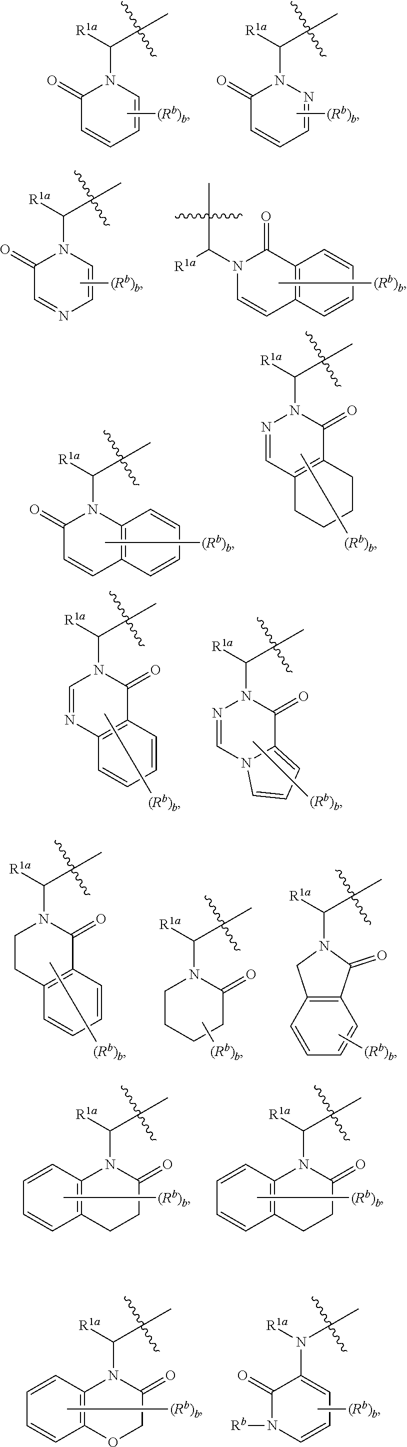

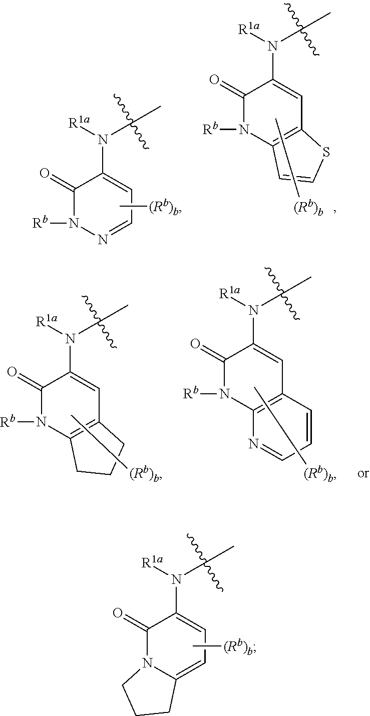

b is selected from 0, 1, 2 or 3;
each R^{b} is independently selected from H, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₂₋₄ alkynyl, C₁₋₄ alkoxy, —C₀₋₄ alkyl-C₃₋₆ carbocyclyl, —C₀₋₄ alkyl-3- to 7-membered heterocyclyl or R^{ba}, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋ₛ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocycle contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl,

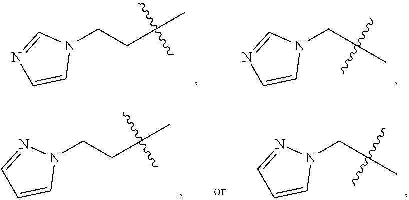 [SMILES: *CCn1ccnc1 ; *Cn1ccnc1 ; *CCn1cccn1 ; *Cn1cccn1]

the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclyl or —C₀₋₂ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclyl contains 1 to 4 heteroatoms selected from O, S or N;
R² is selected from C₁₋₄ alkyl, a benzene ring group, a naphthalene ring group, a 5- to 6-membered heteroaromatic ring group, a 9- to 10-membered heteroaromatic ring group, C₃₋₁₀ non-aromatic carbocyclyl or 5- to 10-membered non-aromatic heterocyclyl, and the R² is optionally substituted with 0 to 4 R^{2a}, wherein the heteroaromatic ring group and heterocyclyl contain 1 to 4 heteroatoms selected from O, S or N;
each R^{2a}, is independently selected from H, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
R³ is selected from H or C₁₋₄ alkyl, wherein the alkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₁₋₄ alkoxy or a C₃₋₆ carbocycle;
ring A is selected from a 5-membered heteroaromatic ring,

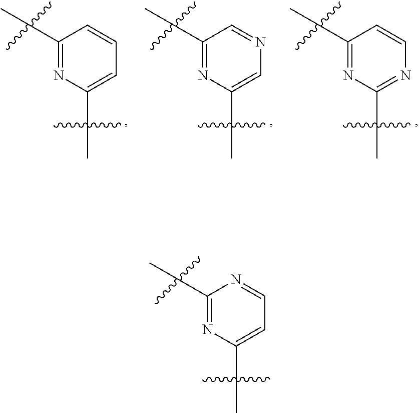 [SMILES: *c1cccc(*)n1 ; *c1cncc(*)n1 ; *c1ccnc(*)n1 ; *c1ccnc(*)n1]

or a 9- to 10-membered fused heteroaromatic ring, and the ring A is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, wherein the heteroaromatic ring contains 1 to 4 heteroatoms selected from N, O or S;
or ring A is selected from

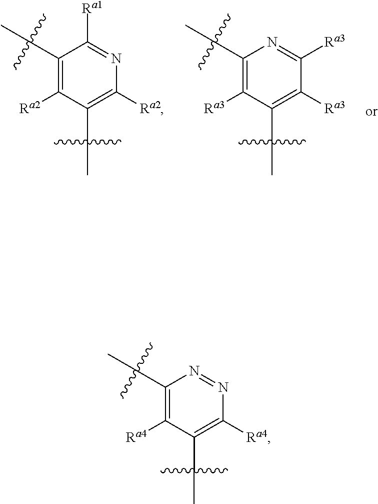

the upper left part of which is directly connected to R²;
R^{a1} is selected from H, halogen, OH, cyano, NH₂, NHC₁₋₄ alkyl, N(C₁₋₄ alkyl)₂, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a2} is independently selected from H, halogen, OH, cyano, NH₂, NHC₁₋₄ alkyl, N(C₁₋₄ alkyl)₂, C₁₋₄ alkyl, C₁₋₄ alkoxy, C₂₋₄ alkenyl, C₂₋₄ alkynyl, C₃₋₆ cycloalkyl, or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄alkoxy, and the heterocyclyl contains 1 to 3 heteroatoms selected from N, O or S;
each R^{a3} is independently selected from H, halogen, OH, cyano, C₁₋₄ alkyl, C₂₋₄ alkenyl, C₂₋₄ alkynyl, C₁₋₄ alkoxy, C₃₋₄ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a4} is independently selected from H, halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S,
provided that 1) when R^{a1} is selected from H, unsubstituted C₁₋₄ alkyl, unsubstituted C₃₋₆ cycloalkyl, or unsubstituted 3- to 7-membered heterocyclyl, R¹ is selected from

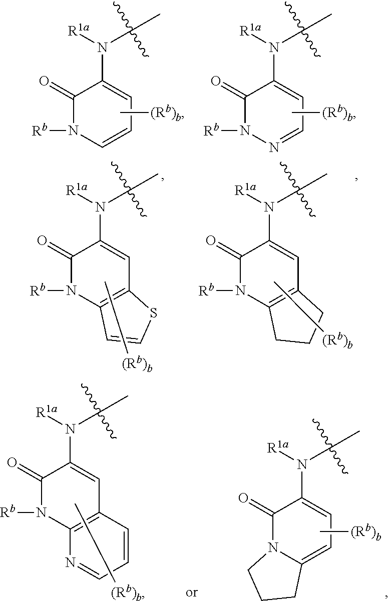

or at least one R^{a2} is not H;
2) when R¹ is selected from

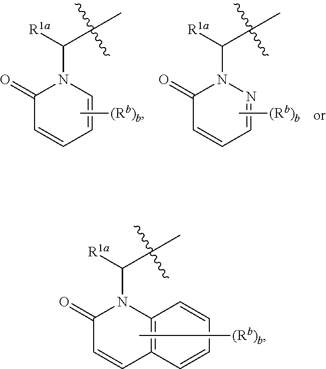

at least one R^{a3} is not H;
3) when R¹ is selected from

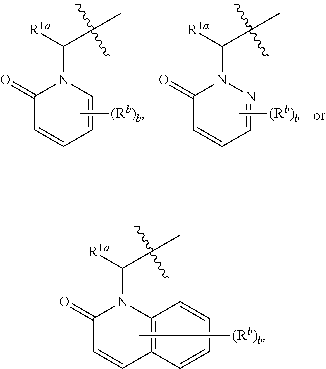

at least one R^{a4} is not H;
or ring A is selected from

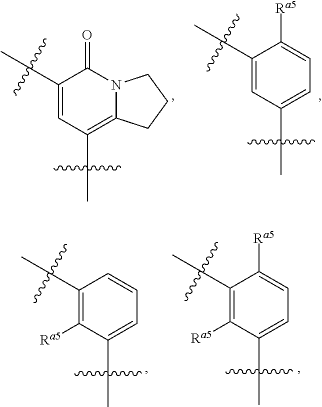

a naphthalene ring or a benzo C₄₋₆ carbocycle, and the ring A is optionally substituted with 0 to 4 R^{a5};
or ring A is selected from

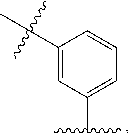 [SMILES: *c1cccc(*)c1]

and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
the definitions of the remaining substituents are consistent with those in the first embodiment of the present disclosure.

As a third embodiment of the present disclosure, provided is the compound as represented by the preceding general formula (I) or a stereoisomer, a deuterated form, a solvate, a prodrug, a metabolite, a pharmaceutically acceptable salt or a co-crystal thereof, wherein
each R^{b} is independently selected from H, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, C₃₋₆ cycloalkyl, 3- to 7-membered heterocycloalkyl, —CH₂—C₃₋₆ cycloalkyl, —CH₂-3- to 7-membered heterocycloalkyl, —CH₂CH₂—C₃₋₆ cycloalkyl, —CH₂CH₂-3- to 7-membered heterocycloalkyl or R^{ba}, wherein the CH₂, alkyl, alkynyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom,

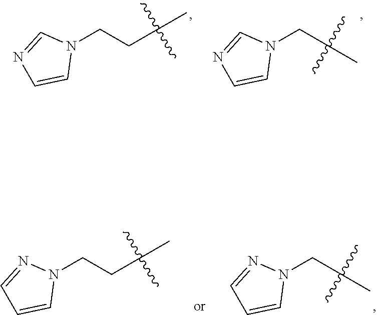 [SMILES: *CCn1ccnc1 ; *Cn1ccnc1 ; *CCn1cccn1 ; *Cn1cccn1]

and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclyl or —C₀₋₄ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
R² is selected from a benzene ring group, a naphthalene ring group, a 5- to 6-membered heteroaromatic ring group, a 9- to 10-membered heteroaromatic ring group, C₃₋₆ cycloalkyl or 3- to 7-membered heterocycloalkyl, and the R² is optionally substituted with 0 to 4 R^{2a}, wherein the heteroaromatic ring group and heterocycloalkyl contain 1 to 4 heteroatoms selected from O, S or N;
each R², is independently selected from H, halogen, OH, cyano, ═O, C₁₋₄ alkyl, C₁₋₄ alkoxy or C₃₋₆ cycloalkyl, wherein the alkyl, alkoxy or cycloalkyl is optionally substituted with 0 to 4 substituents selected from H, halogen, OH, ═O, cyano, C₁₋₄ alkyl or C₁₋₄ alkoxy; R³ is selected from H or C₁₋₄ alkyl, wherein the alkyl is optionally substituted with 0 to 4 substituents selected from H, halogen, C₁₋₄ alkyl, C₁₋₄ alkoxy or a benzene ring group;
ring A is selected from a 5-membered heteroaromatic ring,

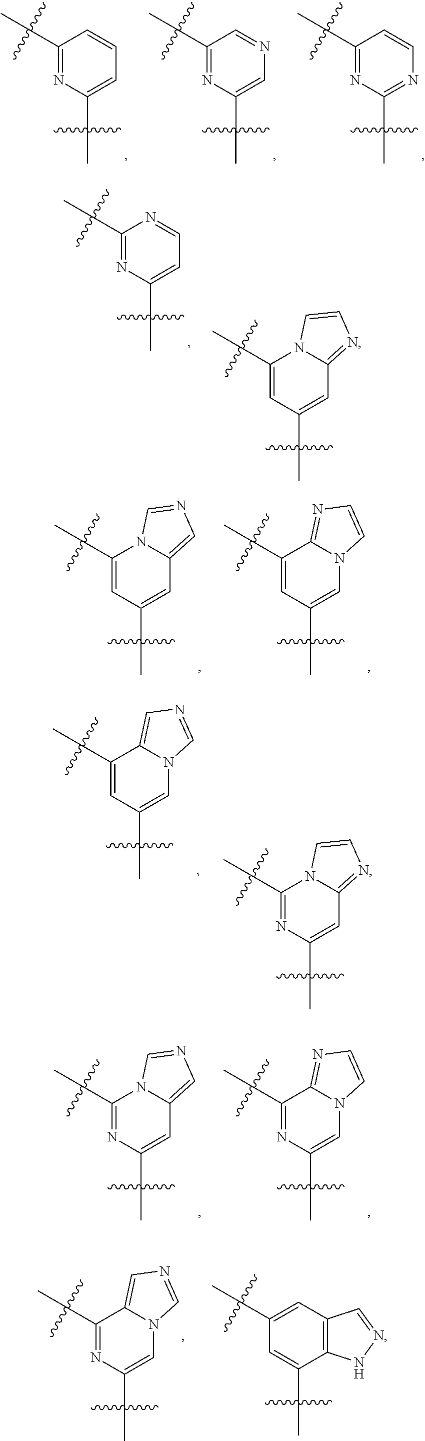

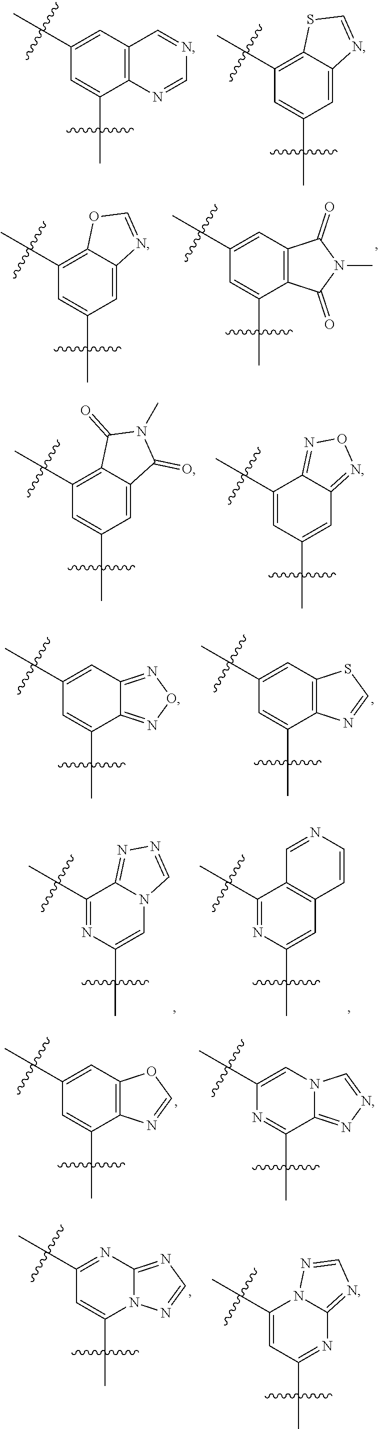

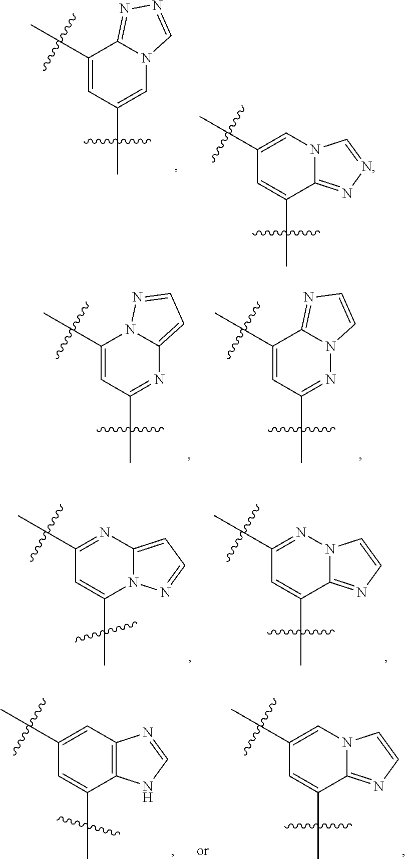

and the ring A is optionally substituted with 0 to 4 substituents selected from H, halogen, OH, cyano, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, wherein the heteroaromatic ring contains 1 to 2 heteroatoms selected from N, O or S;
or ring A is selected from

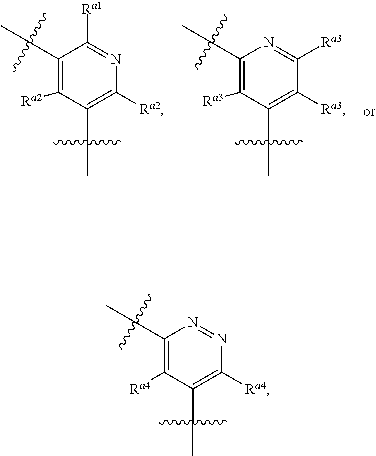

the upper left part of which is directly connected to R²;
R^{a1} is selected from H, halogen, OH, cyano, C₂₋₄ alkenyl, C₂₋₄ alkynyl or C₁₋₄ alkoxy, wherein the alkenyl, alkynyl, or alkoxy is optionally substituted with 0 to 4 substituents selected from halogen, OH, ═O, cyano, C₁₋₄ alkyl or C₁₋₄ alkoxy;
each R^{a2} is independently selected from H, halogen, OH, cyano, C₁₋₄ alkyl or C₁₋₄ alkoxy, wherein the alkyl or alkoxy is optionally substituted with 0 to 4 substituents selected from halogen, OH, ═O, cyano, C₁₋₄ alkyl or C₁₋₄ alkoxy;
each R^{a3} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy;
each R^{a4} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy;
provided that 1) when R^{a1} is selected from H, R¹ is selected from

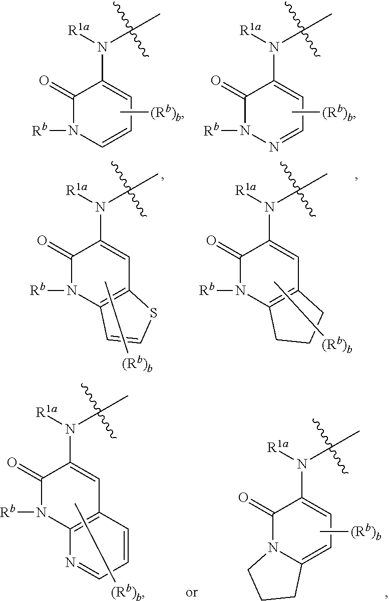

or at least one R^{a2} is not H;
2) when R¹ is selected from or

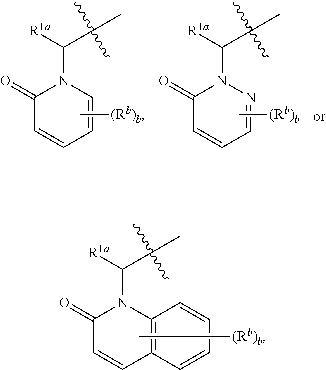

at least one R^{a3} is not H;
3) when R is selected from

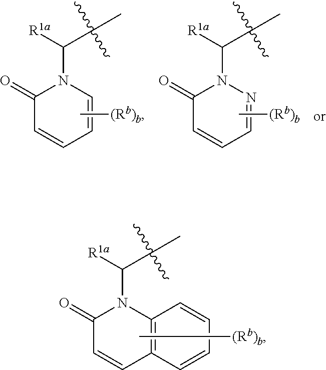

at least one is not H;
or ring A is selected from

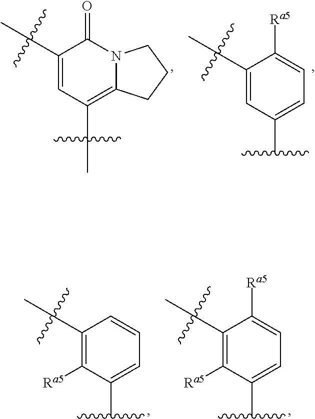

a naphthalene ring,

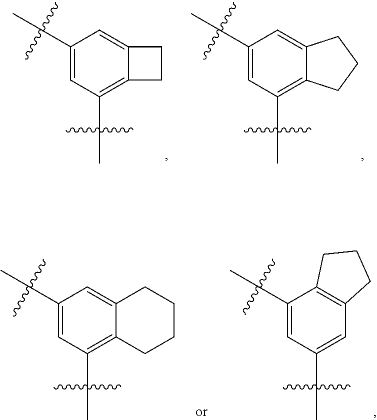 [SMILES: *c1cc(*)c2c(c1)CC2 ; *c1cc(*)c2c(c1)CCC2 ; *c1cc(*)c2c(c1)CCCC2 ; *c1cc(*)c2c(c1)CCC2]

and the ring A is optionally substituted with 0 to 4 R^{a5};
or ring A is selected from

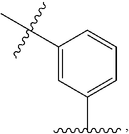 [SMILES: *c1cccc(*)c1]

and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from halogen, OH, ═O, cyano, C₁₋₄ alkyl, C₁₋₄ alkoxy or C₃₋₆ cycloalkyl;
the definitions of the remaining substituents are the same as those in any one of the first or second embodiment of the present disclosure.

As a fourth embodiment of the present disclosure, provided is the compound as represented by the preceding general formula (I) or a stereoisomer, a deuterated form, a solvate, a prodrug, a metabolite, a pharmaceutically acceptable salt or a co-crystal thereof, wherein
R^{1a} is selected from methyl, ethyl, propyl, butyl, isobutyl, sec-butyl, tert-butyl, —CH₂-cyclopropyl or —CH₂-cyclobutyl;
each R^{b} is independently selected from H, F, Cl, Br, I, OH, ═O, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂—cyclobutyl, —CH₂-cyclopentyl, —CH₂-cyclohexyl, —CH₂-azetidinyl, —CH₂-azacyclopentyl, —CH₂-azacyclohexyl, —CH₂-oxetanyl, —CH₂-oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is optionally substituted with 0 to 4 substituents selected from H, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{ba} is selected from one of the following optionally substituted groups:

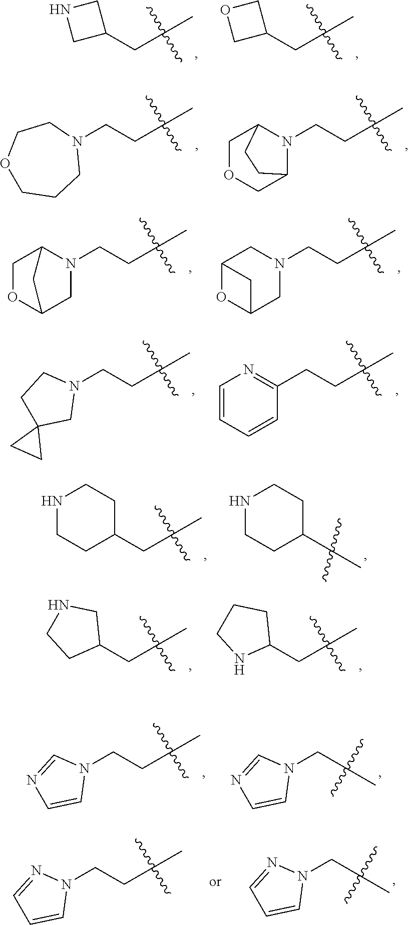

which, when substituted, is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄alkoxy or C₁₋₄ alkoxyalkyl;
R² is selected from a benzene ring group, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl or

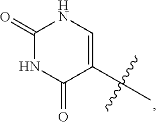 [SMILES: *c1c[nH]c(=O)[nH]c1=O]

and the R² is optionally substituted with 0 to 4 R^{2a};
each R^{2a} is independently selected from H, F, Cl, Br, I, OH, ═O, cyano, methyl, ethyl, methoxy, ethoxy, cyclopropyl or cyclobutyl, wherein the methyl, ethyl, methoxy, ethoxy, cyclopropyl or cyclobutyl is optionally substituted with 0 to 4 substituents selected from H, halogen, OH, ═O, cyano, C₁₋₄ alkyl or C₁₋₄ alkoxy;
R³ is selected from H, methyl, ethyl, propyl, butyl, isobutyl, sec-butyl, tert-butyl or benzyl;
ring A is selected from thiophene, thiazole, furan, oxazole, pyrrole, pyrazole, imidazole,

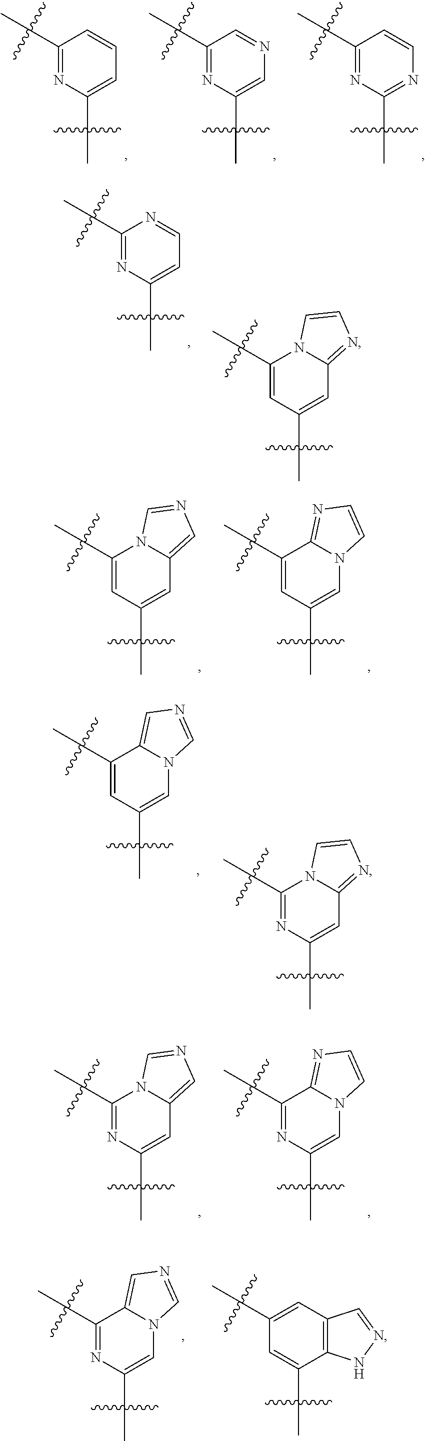

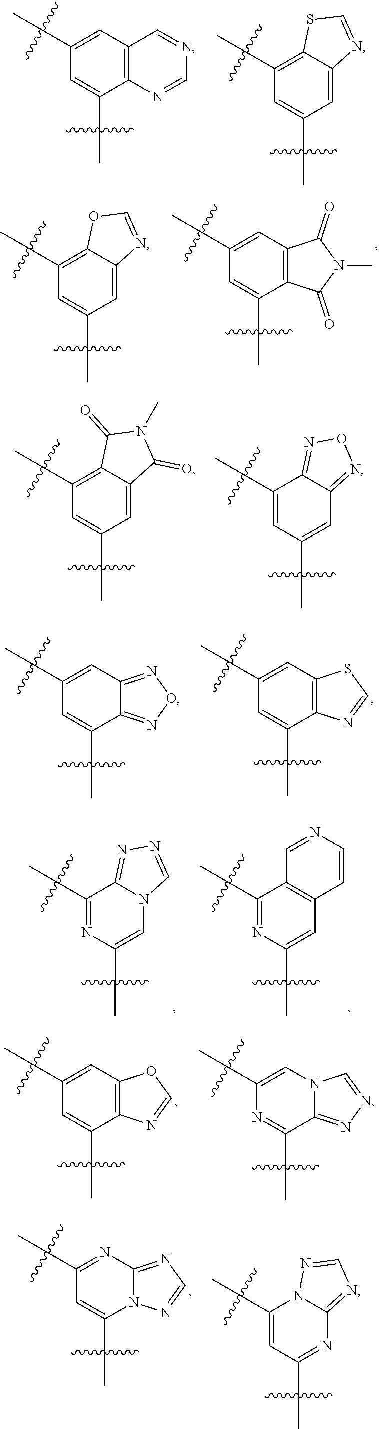

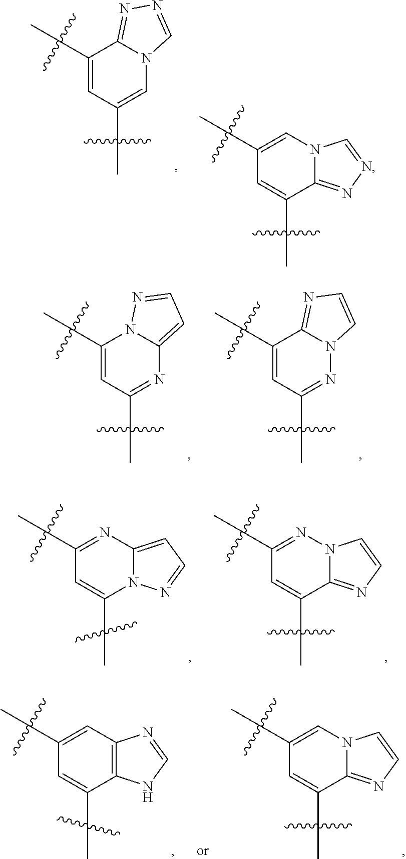

and the ring A is optionally substituted with 0 to 4 substituents selected from H, halogen, OH, cyano, C₁₋₄ alkyl, halogen-substituted C₁₋₄ alkyl, cyano-substituted C₁₋₄ alkyl, C₁₋₄ alkoxy or C₃₋₆ cycloalkyl;
or ring A is selected from

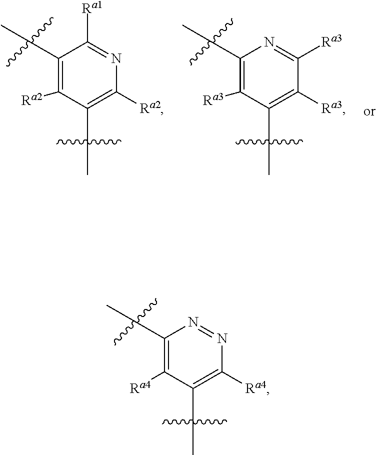

the upper left part of which is directly connected to R²;
R^{a1} is selected from H, F, Cl, Br, I, OH, cyano, ethenyl, ethynyl, methoxy or ethoxy, wherein the ethenyl, ethynyl, methoxy or ethoxy is optionally substituted with 0 to 4 substituents selected from halogen, OH, ═O, cyano, C₁₋₄ alkyl or C₁₋₄ alkoxy;
each R^{a2} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from halogen, OH, ═O, cyano, C₁₋₄ alkyl or C₁₋₄ alkoxy;
each R^{a3} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
each R^{a4} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
or ring A is selected from

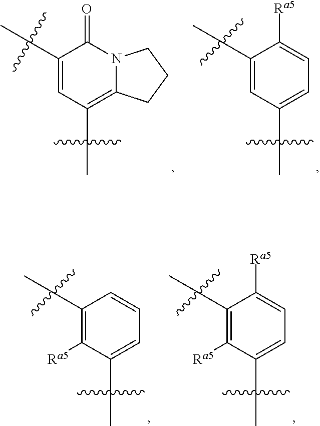

a naphthalene ring,

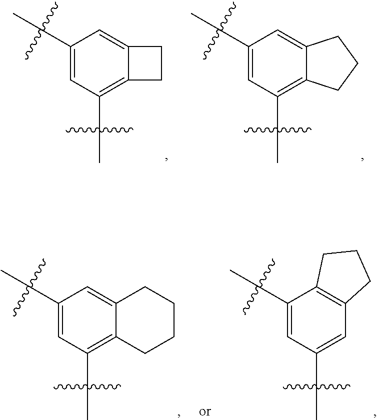 [SMILES: *c1cc(*)c2c(c1)CC2 ; *c1cc(*)c2c(c1)CCC2 ; *c1cc(*)c2c(c1)CCCC2 ; *c1cc(*)c2c(c1)CCC2]

and the ring A is optionally substituted with 0 to 4 R^{a5};
or ring A is selected from

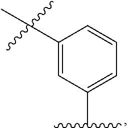 [SMILES: *c1cccc(*)c1]

and the ring A is substituted with one substituent selected from ethenyl, ethynyl, propynyl or propargyl and optionally substituted with 1 to 3 substituents selected from R^{a5};
each R^{a5} is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from halogen, OH, ═O, cyano, C₁₋₄ alkyl, C₁₋₄ alkoxy or C₃₋₆ cycloalkyl; the definitions of the remaining substituents are the same as those in any one of the first, second or third embodiment of the present disclosure.

As a fifth embodiment of the present disclosure, provided is the compound as represented by the preceding general formula (I) or a stereoisomer, a deuterated form, a solvate, a prodrug, a metabolite, a pharmaceutically acceptable salt or a co-crystal thereof, wherein
R¹ is selected from

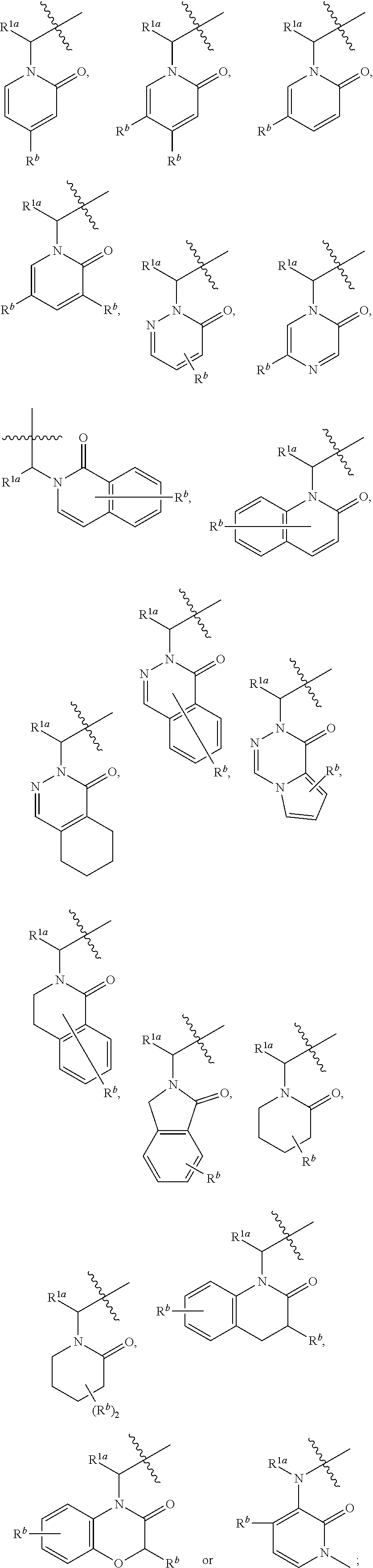

each R^{b} is independently selected from H, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azacyclopentyl, —CH₂-azacyclohexyl, —CH₂-oxetanyl, —CH₂— oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is optionally substituted with 0 to 4 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{ba} is selected from one of the following optionally substituted groups:

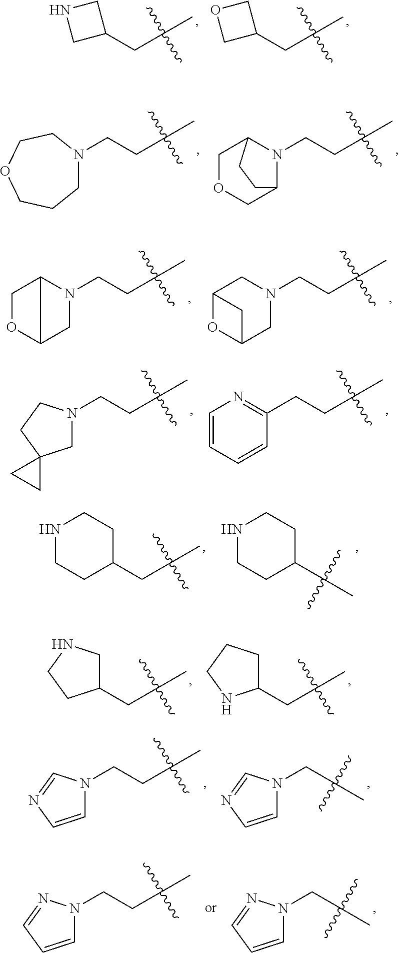

which, when substituted, is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, methoxy, ethoxy, methoxymethyl, ethoxymethyl or methoxyethyl;
R² is selected from phenyl, pyridyl, pyridonyl, azacyclopentyl, morpholinyl or

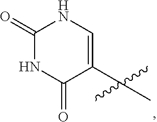 [SMILES: *c1c[nH]c(=O)[nH]c1=O]

and the R² is optionally substituted with 0 to 4 substituents selected from H, F, Cl, Br, OH, CF₃, cyano, methyl, ethyl, methoxy, ethoxy, cyclopropyl or cyclobutyl;
ring A is selected from

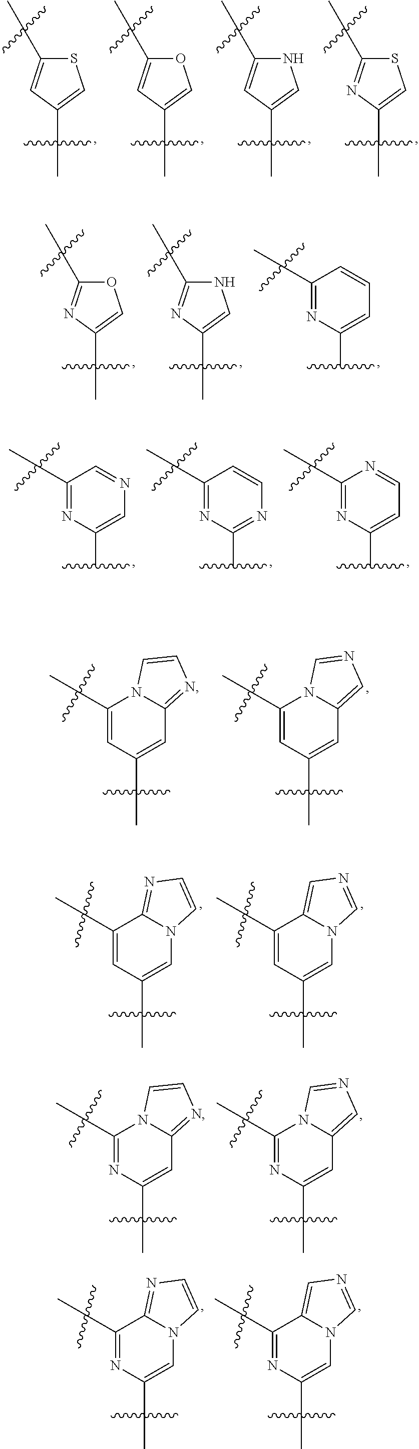

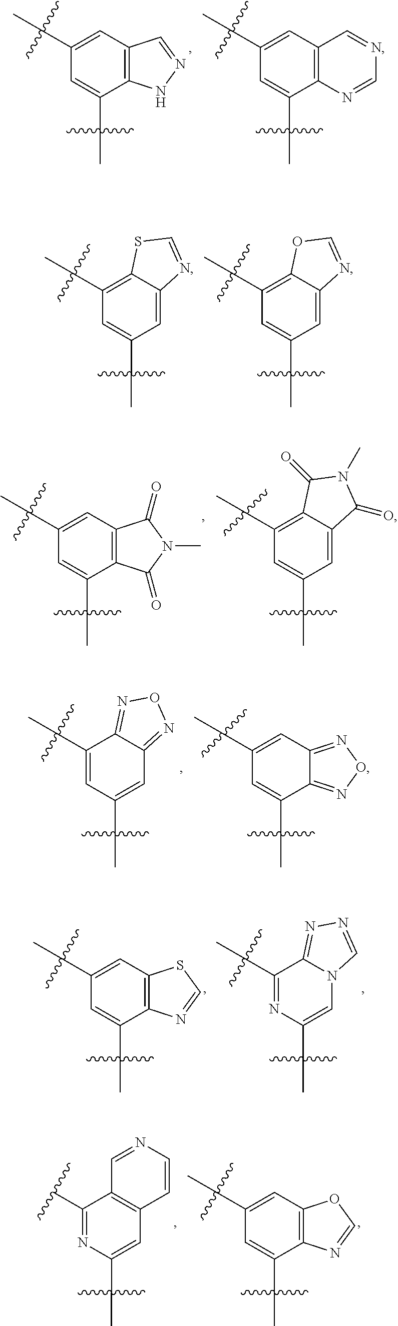

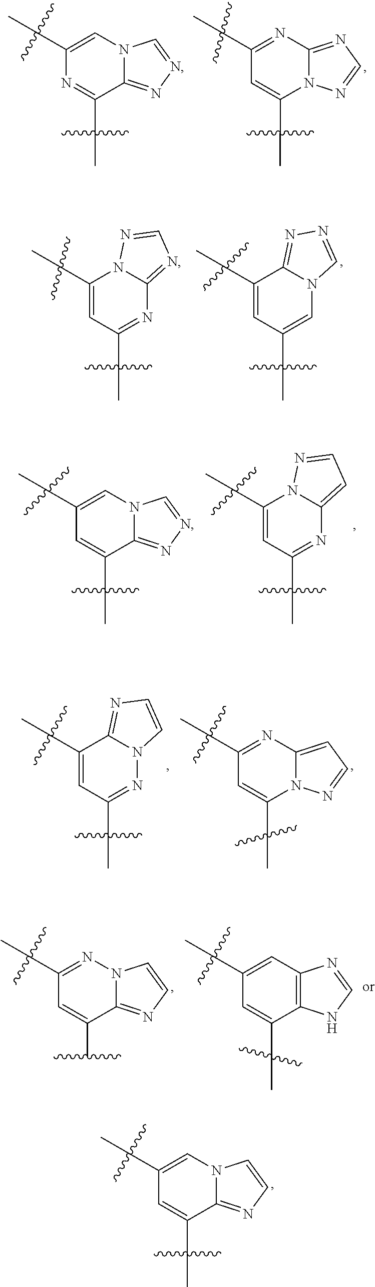

and the ring A is optionally substituted with 0 to 4 substituents selected from H, F, Cl, Br, OH, cyano, CF₃, methyl, ethyl, methoxy, ethoxy, cyclopropyl or cyclobutyl;
or ring A is selected from

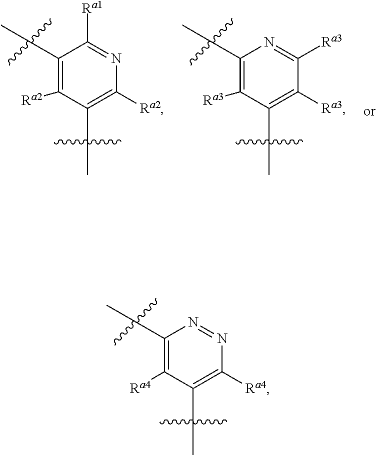

the upper left part of which is directly connected to R².

R^{a1} is selected from H, F, Cl, Br, I, OH, cyano, ethenyl, ethynyl, methoxy, —OCF₃ or ethoxy;
each R^{a2} is independently selected from H, F, Cl, Br, I, OH, cyano, methyl, ethyl, methoxy, —OCF₃ or ethoxy;
each R^{a3} is independently selected from H, F, Cl, Br, I, OH, cyano, methyl, ethyl, methoxy, —OCF₃ or ethoxy;
each R^{a4} is independently selected from H, F, Cl, Br, I, OH, cyano, methyl, ethyl, methoxy, —OCF₃ or ethoxy;
or ring A is selected from

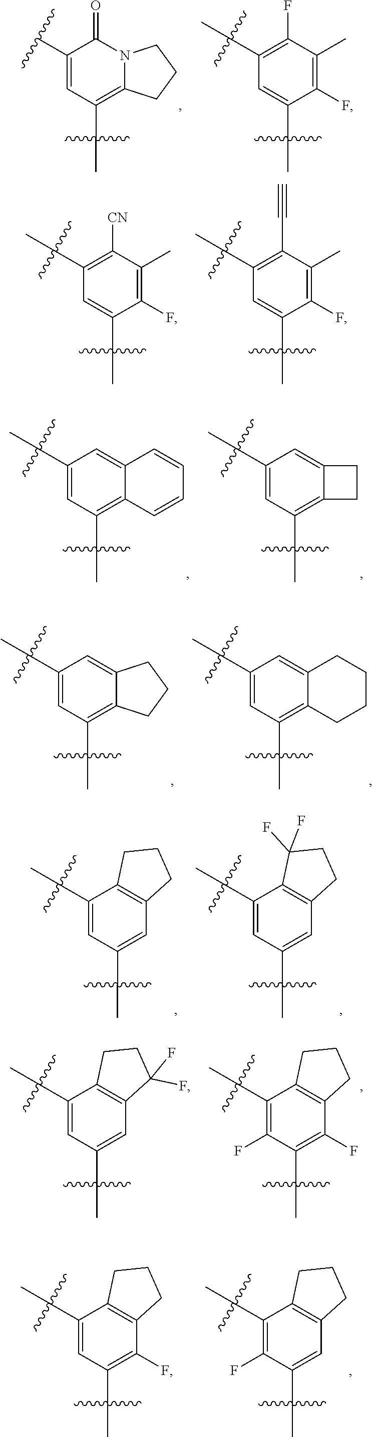

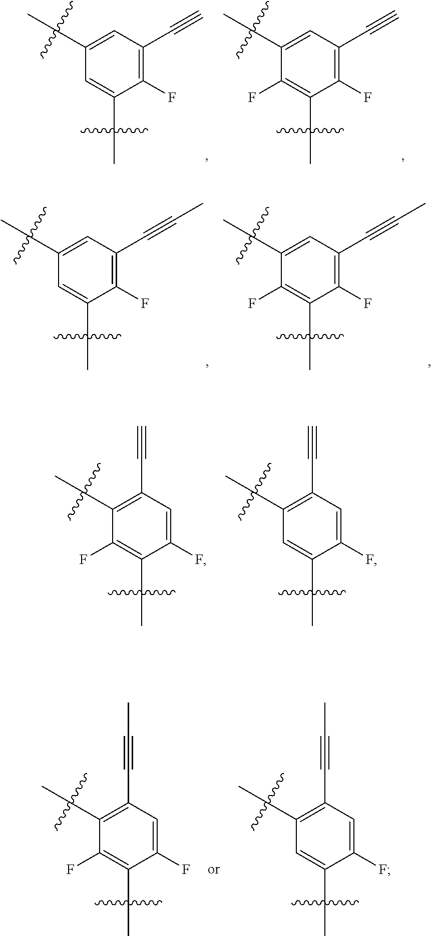

the definitions of the remaining substituents are the same as those in any one of the first, second, third or fourth embodiment of the present disclosure.

As a sixth embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein
R¹ is selected from

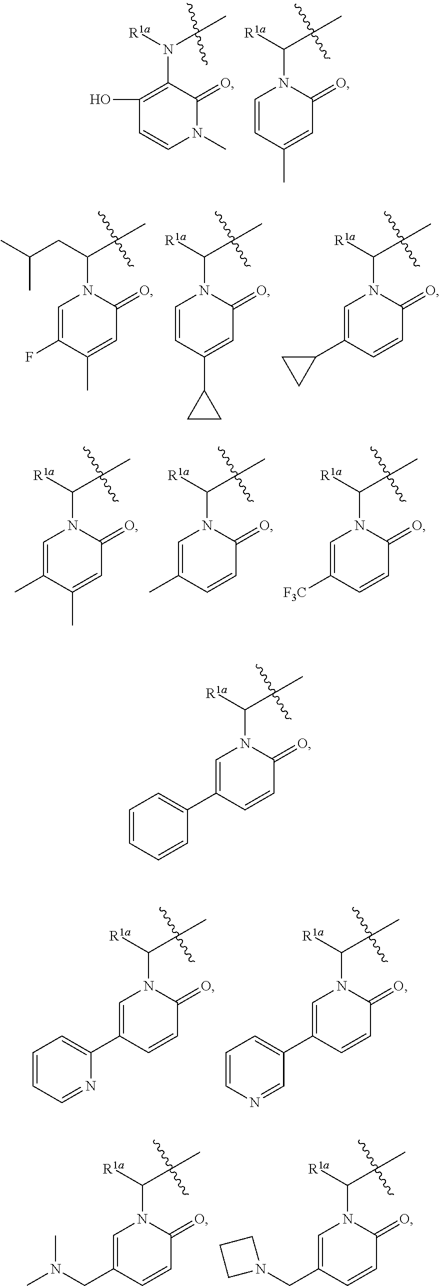

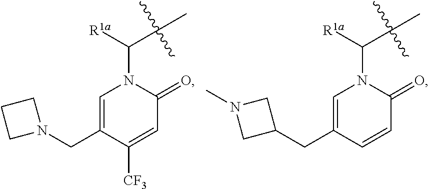

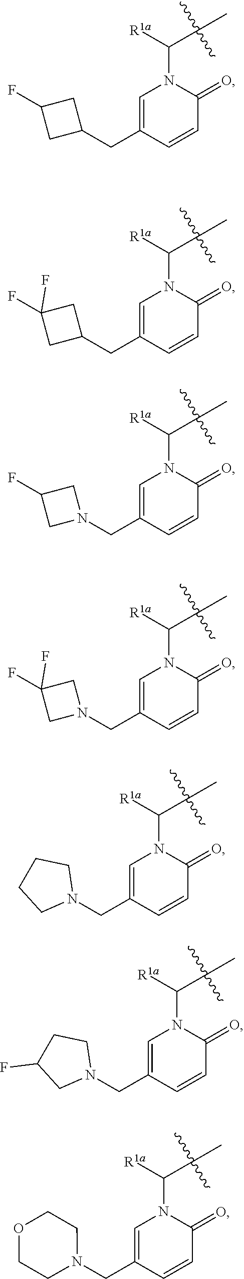

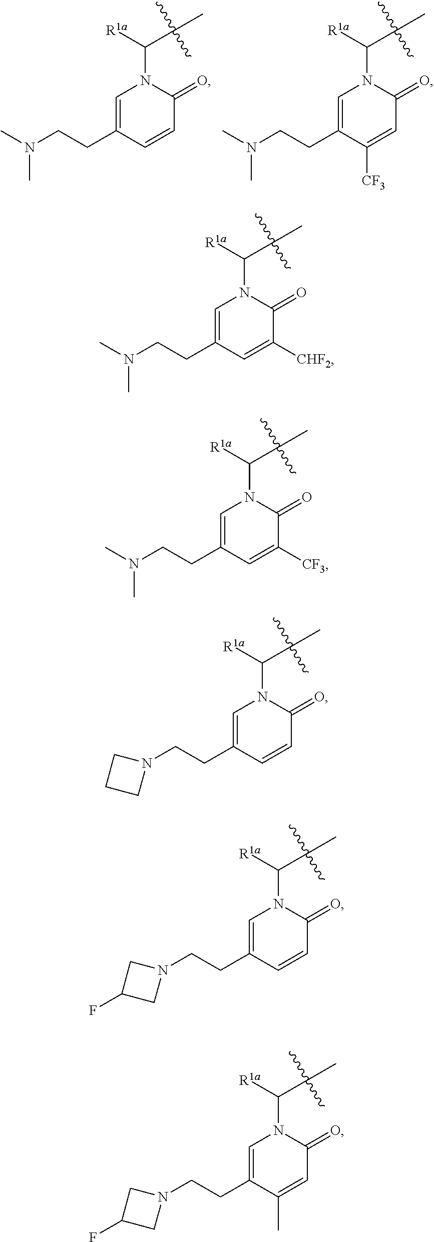

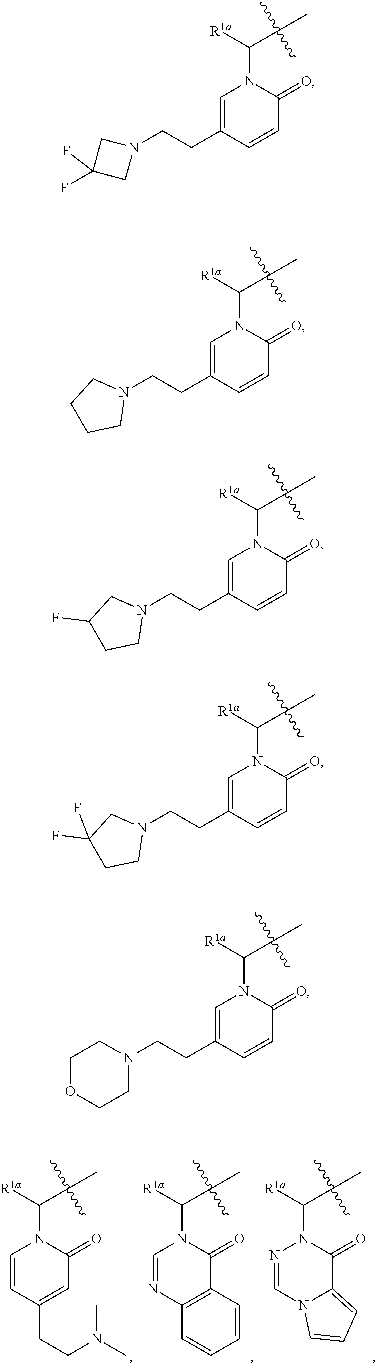

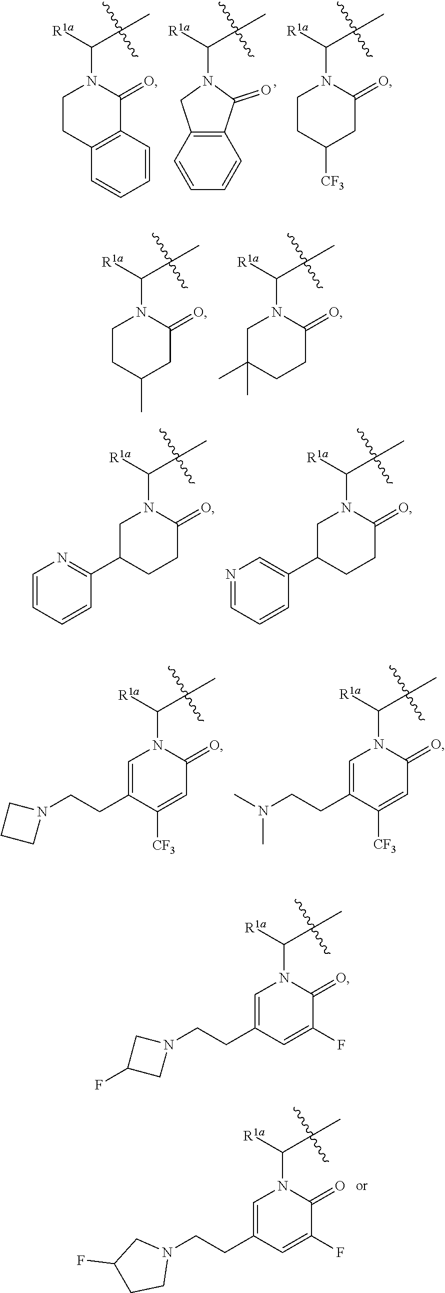

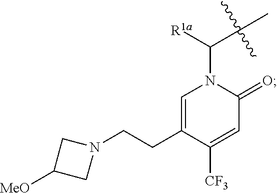

or is selected from

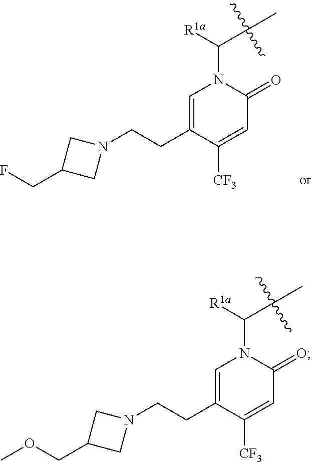

or R¹ is selected from

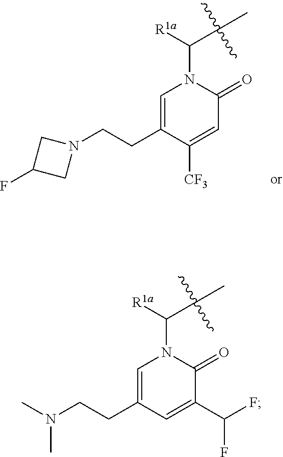

or R¹ is selected from

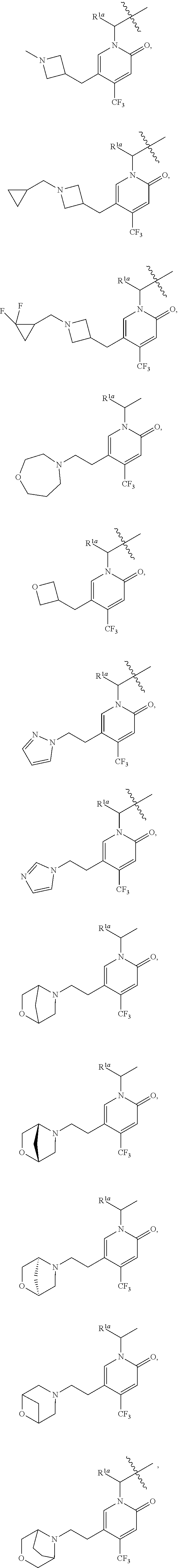

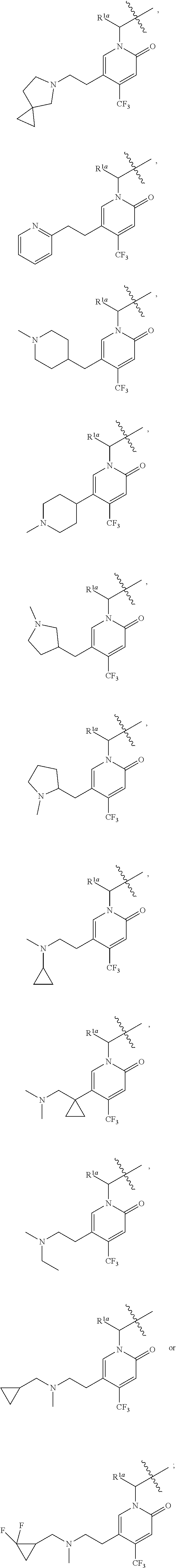

or R¹ is selected from

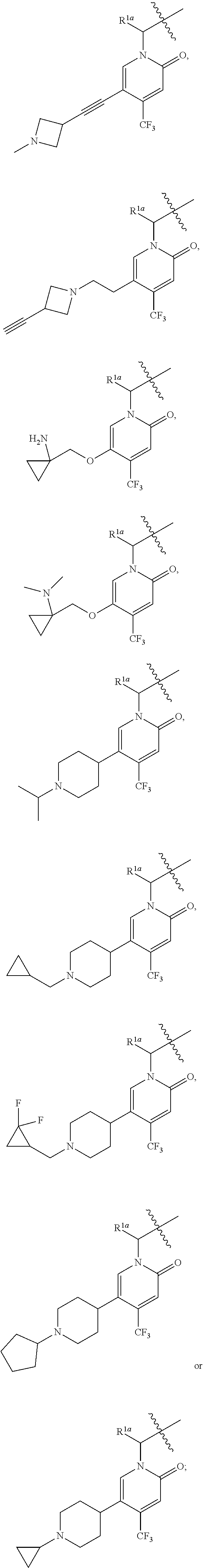

R² is selected from

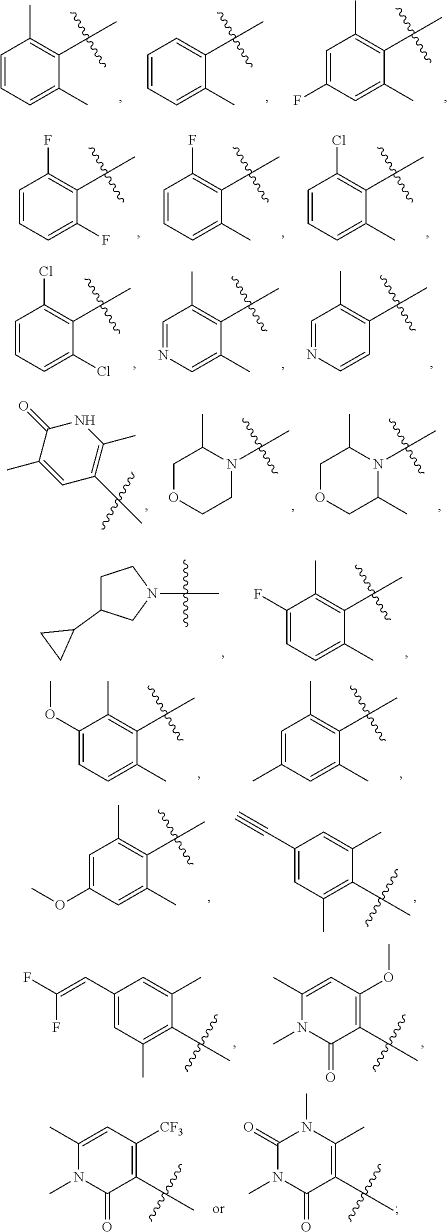

R^{1a} is selected from

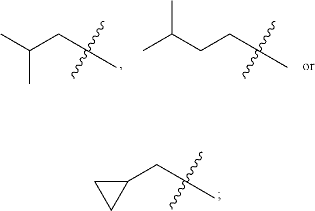

the definitions of the remaining substituents are the same as those in any one of the first, second, third, fourth or fifth embodiment of the present disclosure.

As a seventh embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof,
wherein ring A is selected from

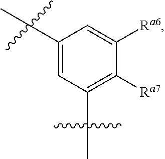

the upper left part of which is directly connected to R²;
R^{a6} and R^{a7} are each independently selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally further substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
R^{1b} is selected from

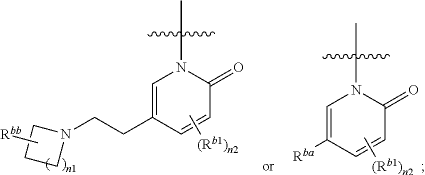

n1 is selected from 1, 2, 3 or 4;
n2 is selected from 0, 1, 2, 3 or 4;
each R^{b1} is independently selected from H, halogen, CN, C₁₋₄ alkyl, or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
R^{bb} is selected from halogen, OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
the definitions of the remaining substituents are the same as those in any one of the first, second, third, fourth, fifth or sixth embodiment of the present disclosure.

As an eighth embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein
R^{a6} and R^{a7} are each independently selected from F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
R^{1b} is selected from

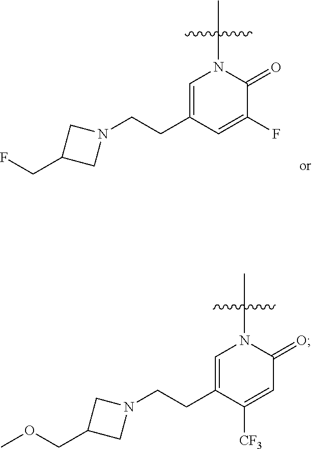 [SMILES: *n1cc(CCN2CC(CF)C2)cc(F)c1=O ; *n1cc(CCN2CC(COC)C2)c(C(F)(F)F)cc1=O]

the definitions of the remaining substituents are the same as those in any one of the first, second, third, fourth, fifth, sixth or seventh embodiment of the present disclosure.

As a ninth embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein
ring A is selected from

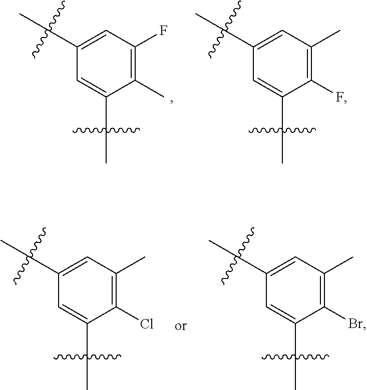

the upper left part of which is directly connected to R²;
the definitions of the remaining substituents are the same as those in any one of the first, second, third, fourth, fifth, sixth, seventh or eighth embodiment of the present disclosure.

As a tenth embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein the compound is selected from a compound as represented by general formula (I-a), (I-b), (I-c), (I-d) or (I-e),

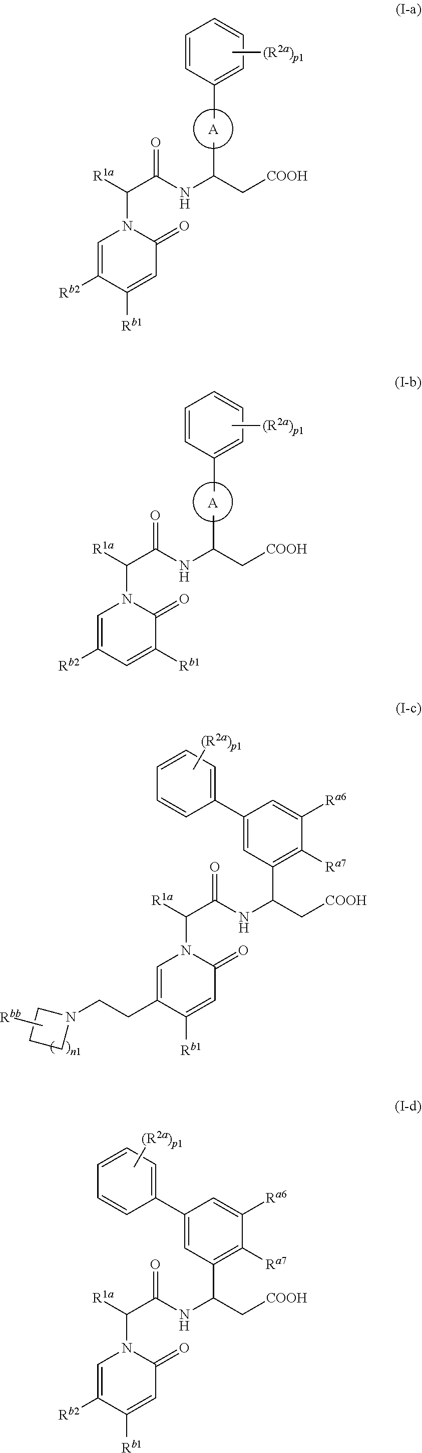

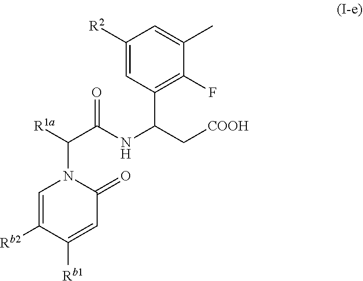

the definition of ring A is the same as that in any one of the second, third, fourth, fifth or sixth embodiment of the present disclosure;
p1 is selected from 0, 1, 2, 3 or 4;
R^{2a} is selected from H, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from halogen, OH, CN, C₁₋₄ alkyl or C₁₋₄ alkoxy, and the heterocycloalkyl contains 1 to 4 heteroatoms selected from O, S or N;
R^{1a} is selected from

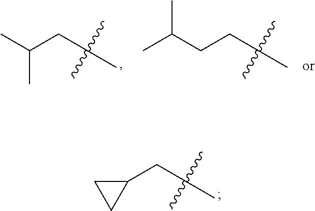

R^{b1} is selected from H, C₁₋₄ alkyl or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{bb} is selected from OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl or C₁₋₄ alkoxy;
R^{a6} and R^{a7} are each independently selected from halogen, OH, cyano, C₁₋₄ alkyl, C₂₋₄ alkynyl or C₁₋₄ alkoxy, wherein the alkyl, alkynyl or alkoxy is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
R² is selected from a 5- to 6-membered heteroaromatic ring group or phenyl, wherein the phenyl is substituted with one R^{2aa} and optionally substituted with 1 to 3 R^{2a}, and the heteroaromatic ring group is optionally substituted with 1 to 4 R^{2a} and the heteroaromatic ring contains 1 to 3 heteroatoms selected from O, S or N;
R^{2aa} is selected from C₂₋₄ alkenyl or C₂₋₄ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
n1 is selected from 1, 2 or 3;
the definitions of R^{ba} and R^{k} are the same as those in any one of the second, third, fourth, fifth or sixth embodiment of the present disclosure;
R^{bb} is selected from OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
R^{a6} and R^{a7} are each independently selected from halogen, OH, cyano, C₁₋₄ alkyl, C₂₋₄ alkynyl or C₁₋₄ alkoxy, wherein the alkyl, alkynyl or alkoxy is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
R² is selected from a 5- to 6-membered heteroaromatic ring group or phenyl, wherein the phenyl is substituted with one R^{2aa} and optionally substituted with 1 to 3 R^{2a}, and the heteroaromatic ring group is optionally substituted with 1 to 4 R^{2a} and contains 1 to 3 heteroatoms selected from O, S or N;
R^{2aa} is selected from C₂₋₄ alkenyl or C₂₋₄ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
n1 is selected from 1, 2 or 3.

As an eleventh embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein the compound is selected from a compound as represented by general formula (I-a), (I-b) or (I-c),
R² is selected from F, Cl, Br, I, OH, cyano, methyl, ethyl, methoxy or ethoxy;
R^{b1} is selected from H, F, CH₂F, CHF₂, CF₃, methyl or —CH₂CH₂N(CH₃)₂;
R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 1, 2 or 3 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{a6} and R^{a7} are each independently selected from F, Cl, Br, I, OH, cyano, methyl, ethyl, methoxy or ethoxy;
R^{bb} is selected from CH₂F, CHF₂, CF₃, methyl, ethyl or methoxymethyl;
R² s selected from

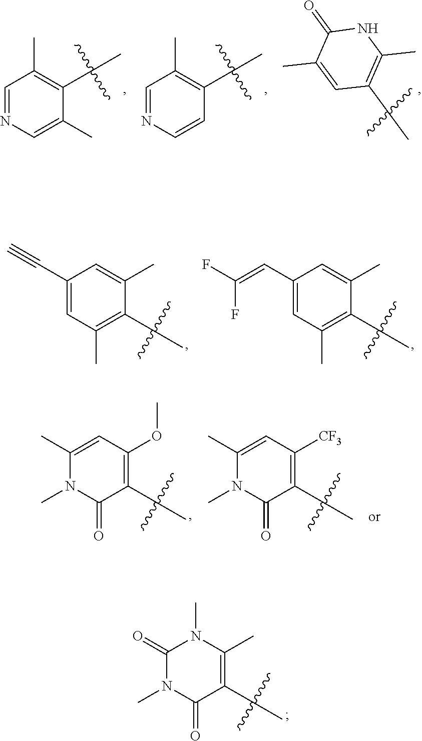

the definitions of the remaining groups are the same as those in the tenth embodiment of the present disclosure.

As an eleventh embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein the compound is selected from a compound as represented by general formula (I-f) or (I-g):

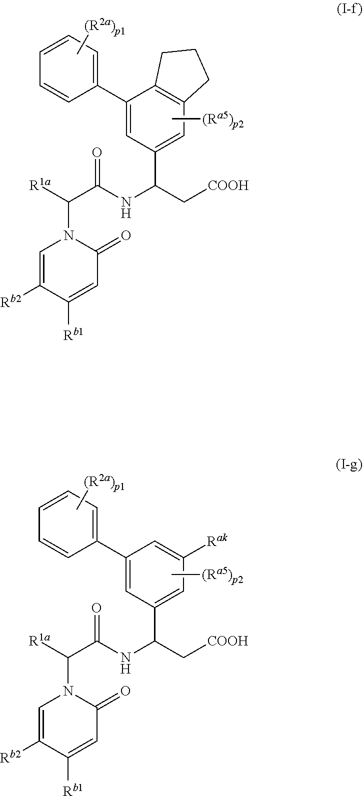

wherein p2 is selected from 0, 1 or 2;
R^{ak} is selected from C₂₋₄ alkynyl;
the definition of R^{a5} is the same as that in any one of the first, second, third or fourth embodiment of the present disclosure;
the definitions of the remaining groups are the same as those in any one of the tenth or eleventh embodiment of the present disclosure.

As a thirteenth embodiment of the present disclosure, provided is the compound as represented by the above-mentioned general formula (I) or a stereoisomer, a deuterated form, a solvate, a prodrug, a metabolite, a pharmaceutically acceptable salt or a co-crystal thereof, wherein the compound is selected from a compound as represented by general formula (I-f) or (I-g):
R^{ak} is selected from ethynyl, propynyl or propargyl;
R^{a5} is selected from F, Cl, Br, I, OH, cyano, methyl, ethyl, methoxy, ethoxy or ethynyl;
the definitions of the remaining groups are the same as those in the twelfth embodiment of the present disclosure.

As an embodiment of the present disclosure, in general formula (I-a), (I-b), (I-c), (I-d), (I-e), (I-f) or (I-g), the carbon atom connected to pyridone is in the R or S configuration, preferably in the S configuration.

As an embodiment of the present disclosure, in general formula (I-a), (I-b), (I-c), (I-d), (I-e), (I-f) or (I-g), the carbon atom connected to —CH₂—COOH is in the R or S configuration, preferably in the S configuration.

The present disclosure relates to some specific compounds of general formula (I), wherein the compounds are selected from table E-1 or table E-2.

TABLE E-1

| | | | |
|---|---|---|---|
| 1 | 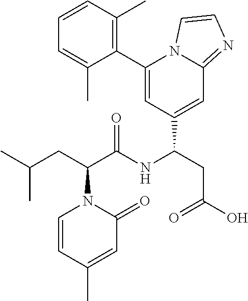 [SMILES: Cc1ccn([C@@H](CC(C)C)C(=O)N[C@@H](CC(=O)O)c2cc(-c3c(C)cccc3C)n3ccnc3c2)c(=O)c1] | 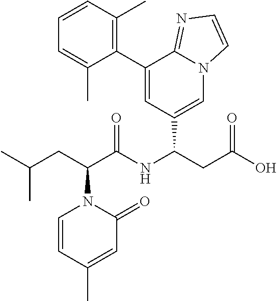 [SMILES: Cc1ccn([C@@H](CC(C)C)C(=O)N[C@@H](CC(=O)O)c2cc(-c3c(C)cccc3C)c3nccn3c2)c(=O)c1] | 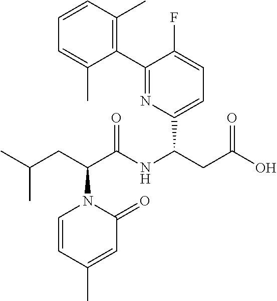 [SMILES: Cc1ccn([C@@H](CC(C)C)C(=O)N[C@@H](CC(=O)O)c2ccc(F)c(-c3c(C)cccc3C)n2)c(=O)c1] |
| 2 | 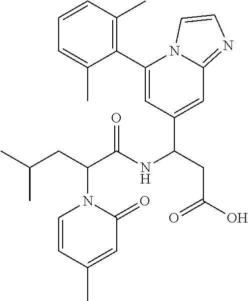 [SMILES: Cc1ccn(C(CC(C)C)C(=O)NC(CC(=O)O)c2cc(-c3c(C)cccc3C)n3ccnc3c2)c(=O)c1] | 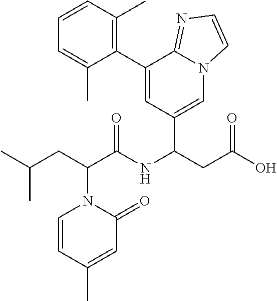 [SMILES: Cc1ccn(C(CC(C)C)C(=O)NC(CC(=O)O)c2cc(-c3c(C)cccc3C)c3nccn3c2)c(=O)c1] | 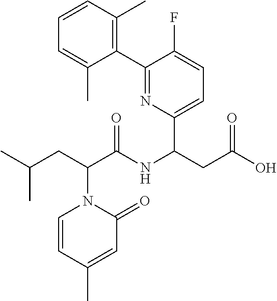 [SMILES: Cc1ccn(C(CC(C)C)C(=O)NC(CC(=O)O)c2ccc(F)c(-c3c(C)cccc3C)n2)c(=O)c1] |
| 3 | 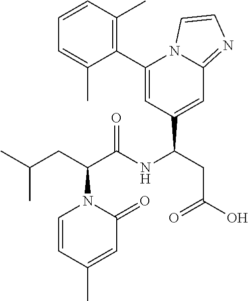 [SMILES: Cc1ccn([C@@H](CC(C)C)C(=O)N[C@H](CC(=O)O)c2cc(-c3c(C)cccc3C)n3ccnc3c2)c(=O)c1] | 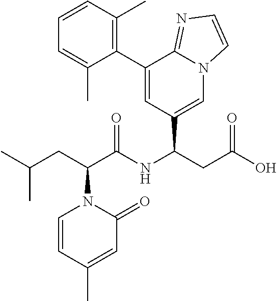 [SMILES: Cc1ccn([C@@H](CC(C)C)C(=O)N[C@H](CC(=O)O)c2cc(-c3c(C)cccc3C)c3nccn3c2)c(=O)c1] | 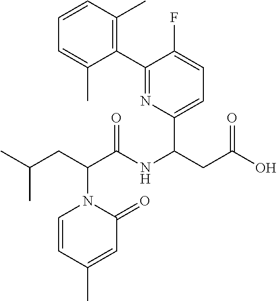 [SMILES: Cc1ccn(C(CC(C)C)C(=O)NC(CC(=O)O)c2ccc(F)c(-c3c(C)cccc3C)n2)c(=O)c1] |
| 4 | 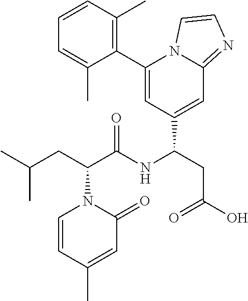 [SMILES: Cc1ccn([C@H](CC(C)C)C(=O)N[C@@H](CC(=O)O)c2cc(-c3c(C)cccc3C)n3ccnc3c2)c(=O)c1] | 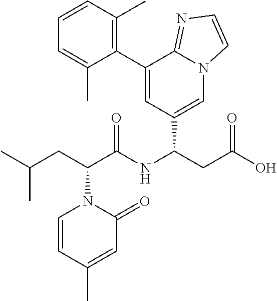 [SMILES: Cc1ccn([C@H](CC(C)C)C(=O)N[C@@H](CC(=O)O)c2cc(-c3c(C)cccc3C)c3nccn3c2)c(=O)c1] | 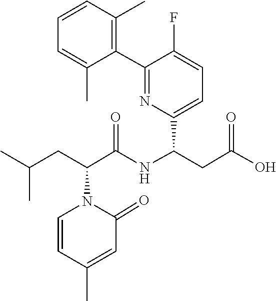 [SMILES: Cc1ccn([C@H](CC(C)C)C(=O)N[C@@H](CC(=O)O)c2ccc(F)c(-c3c(C)cccc3C)n2)c(=O)c1] |
| 5 | 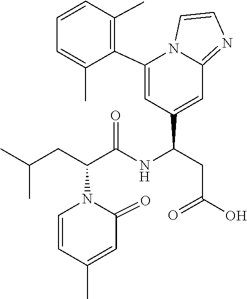 [SMILES: Cc1ccn([C@H](CC(C)C)C(=O)N[C@H](CC(=O)O)c2cc(-c3c(C)cccc3C)n3ccnc3c2)c(=O)c1] | 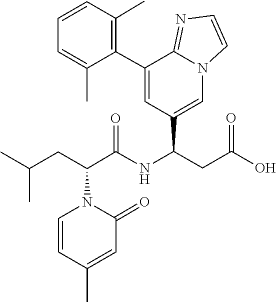 [SMILES: Cc1ccn([C@H](CC(C)C)C(=O)N[C@H](CC(=O)O)c2cc(-c3c(C)cccc3C)c3nccn3c2)c(=O)c1] | 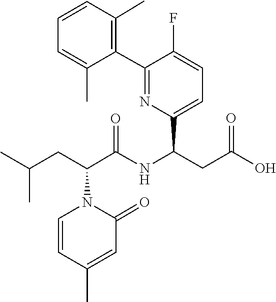 [SMILES: Cc1ccn([C@H](CC(C)C)C(=O)N[C@H](CC(=O)O)c2ccc(F)c(-c3c(C)cccc3C)n2)c(=O)c1] |
| 6 | 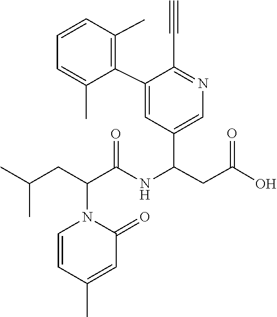 [SMILES: C#Cc1ncc(C(CC(=O)O)NC(=O)C(CC(C)C)n2ccc(C)cc2=O)cc1-c1c(C)cccc1C] | 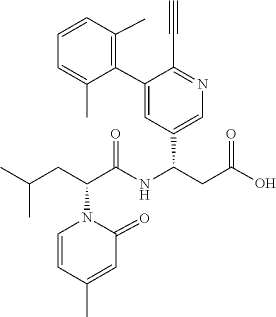 [SMILES: C#Cc1ncc([C@H](CC(=O)O)NC(=O)[C@@H](CC(C)C)n2ccc(C)cc2=O)cc1-c1c(C)cccc1C] | |
| 7 | 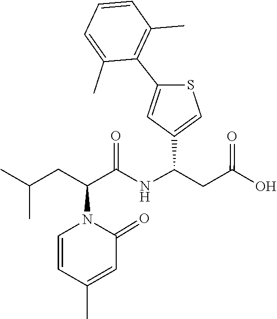 [SMILES: Cc1ccn([C@@H](CC(C)C)C(=O)N[C@@H](CC(=O)O)c2csc(-c3c(C)cccc3C)c2)c(=O)c1] | 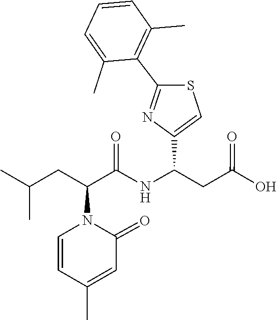 [SMILES: Cc1ccn([C@@H](CC(C)C)C(=O)N[C@@H](CC(=O)O)c2csc(-c3c(C)cccc3C)n2)c(=O)c1] | 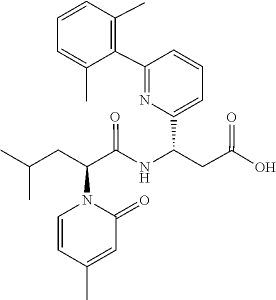 [SMILES: Cc1ccn([C@@H](CC(C)C)C(=O)N[C@@H](CC(=O)O)c2cccc(-c3c(C)cccc3C)n2)c(=O)c1] |
| 8 | 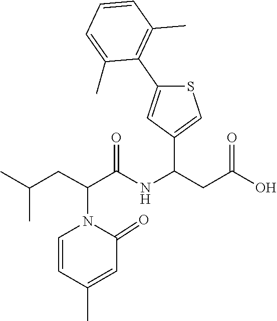 [SMILES: Cc1ccn(C(CC(C)C)C(=O)NC(CC(=O)O)c2csc(-c3c(C)cccc3C)c2)c(=O)c1] | 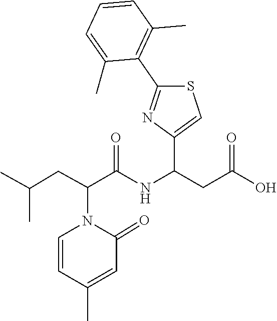 [SMILES: Cc1ccn(C(CC(C)C)C(=O)NC(CC(=O)O)c2csc(-c3c(C)cccc3C)n2)c(=O)c1] | 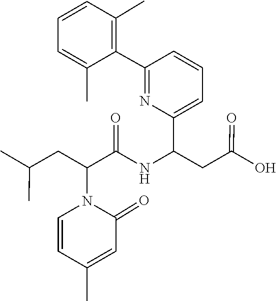 [SMILES: Cc1ccn(C(CC(C)C)C(=O)NC(CC(=O)O)c2cccc(-c3c(C)cccc3C)n2)c(=O)c1] |
| 9 | 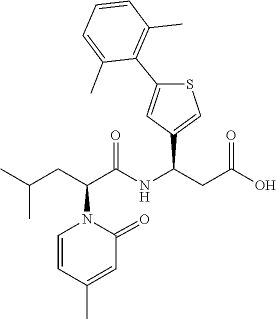 [SMILES: Cc1ccn([C@@H](CC(C)C)C(=O)N[C@H](CC(=O)O)c2csc(-c3c(C)cccc3C)c2)c(=O)c1] | 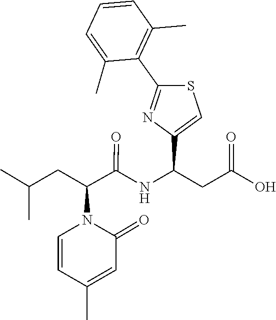 [SMILES: Cc1ccn([C@@H](CC(C)C)C(=O)N[C@H](CC(=O)O)c2csc(-c3c(C)cccc3C)n2)c(=O)c1] | 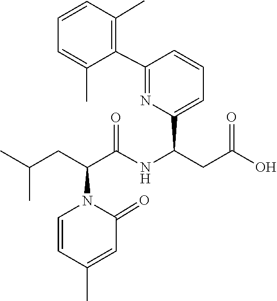 [SMILES: Cc1ccn([C@@H](CC(C)C)C(=O)N[C@H](CC(=O)O)c2cccc(-c3c(C)cccc3C)n2)c(=O)c1] |
| 10 | 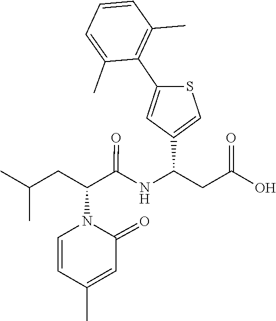 [SMILES: Cc1ccn([C@H](CC(C)C)C(=O)N[C@@H](CC(=O)O)c2csc(-c3c(C)cccc3C)c2)c(=O)c1] | 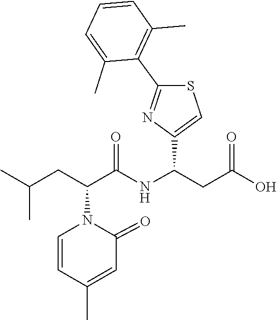 [SMILES: Cc1ccn([C@H](CC(C)C)C(=O)N[C@@H](CC(=O)O)c2csc(-c3c(C)cccc3C)n2)c(=O)c1] | 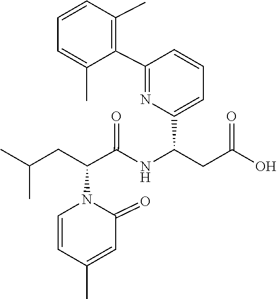 [SMILES: Cc1ccn([C@H](CC(C)C)C(=O)N[C@@H](CC(=O)O)c2cccc(-c3c(C)cccc3C)n2)c(=O)c1] |
| 11 | 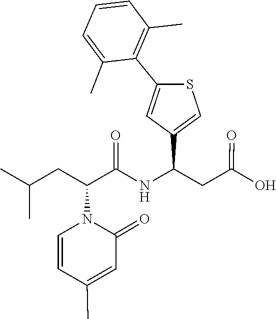 [SMILES: Cc1ccn([C@H](CC(C)C)C(=O)N[C@H](CC(=O)O)c2csc(-c3c(C)cccc3C)c2)c(=O)c1] | 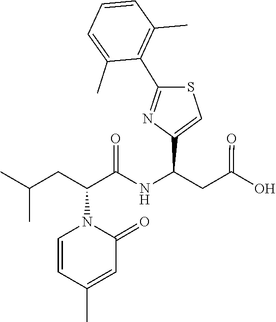 [SMILES: Cc1ccn([C@H](CC(C)C)C(=O)N[C@H](CC(=O)O)c2csc(-c3c(C)cccc3C)n2)c(=O)c1] | 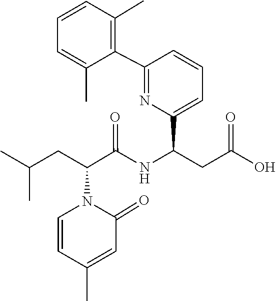 [SMILES: Cc1ccn([C@H](CC(C)C)C(=O)N[C@H](CC(=O)O)c2cccc(-c3c(C)cccc3C)n2)c(=O)c1] |
| 12 | 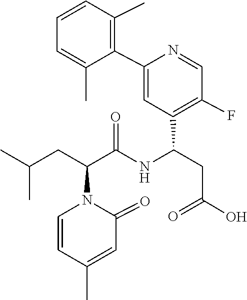 [SMILES: Cc1ccn([C@@H](CC(C)C)C(=O)N[C@@H](CC(=O)O)c2cc(-c3c(C)cccc3C)ncc2F)c(=O)c1] | 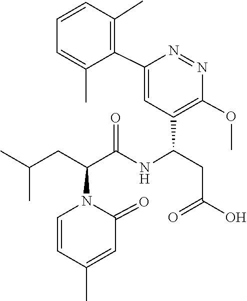 [SMILES: COc1nnc(-c2c(C)cccc2C)cc1[C@H](CC(=O)O)NC(=O)[C@H](CC(C)C)n1ccc(C)cc1=O] | 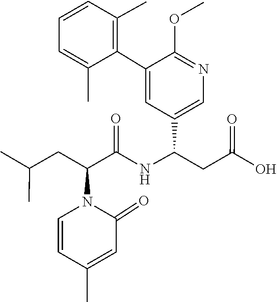 [SMILES: COc1ncc([C@H](CC(=O)O)NC(=O)[C@H](CC(C)C)n2ccc(C)cc2=O)cc1-c1c(C)cccc1C] |
| 13 | 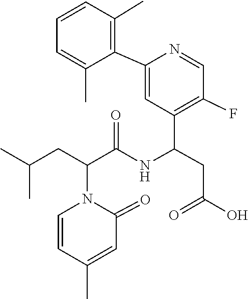 [SMILES: Cc1ccn(C(CC(C)C)C(=O)NC(CC(=O)O)c2cc(-c3c(C)cccc3C)ncc2F)c(=O)c1] | 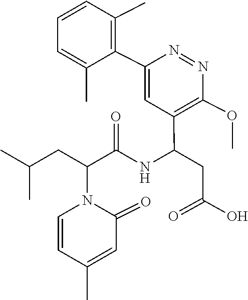 [SMILES: COc1nnc(-c2c(C)cccc2C)cc1C(CC(=O)O)NC(=O)C(CC(C)C)n1ccc(C)cc1=O] | 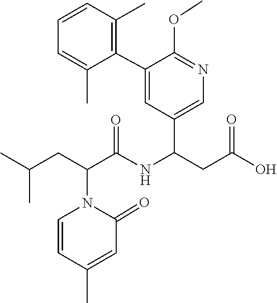 [SMILES: COc1ncc(C(CC(=O)O)NC(=O)C(CC(C)C)n2ccc(C)cc2=O)cc1-c1c(C)cccc1C] |
| 14 | 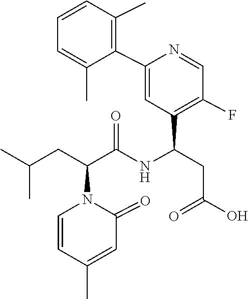 [SMILES: Cc1ccn([C@@H](CC(C)C)C(=O)N[C@H](CC(=O)O)c2cc(-c3c(C)cccc3C)ncc2F)c(=O)c1] | 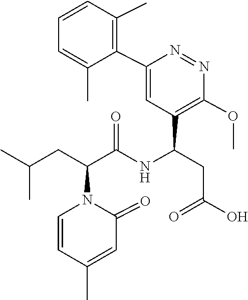 [SMILES: COc1nnc(-c2c(C)cccc2C)cc1[C@@H](CC(=O)O)NC(=O)[C@H](CC(C)C)n1ccc(C)cc1=O] | 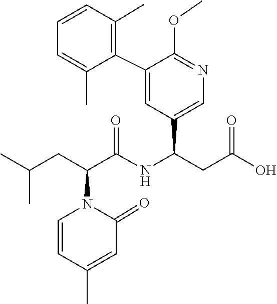 [SMILES: COc1ncc([C@@H](CC(=O)O)NC(=O)[C@H](CC(C)C)n2ccc(C)cc2=O)cc1-c1c(C)cccc1C] |
| 15 | 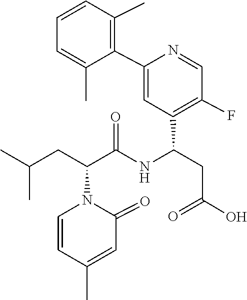 [SMILES: Cc1ccn([C@H](CC(C)C)C(=O)N[C@@H](CC(=O)O)c2cc(-c3c(C)cccc3C)ncc2F)c(=O)c1] | 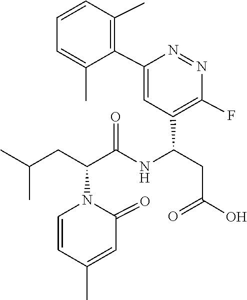 [SMILES: Cc1ccn([C@H](CC(C)C)C(=O)N[C@@H](CC(=O)O)c2cc(-c3c(C)cccc3C)nnc2F)c(=O)c1] | 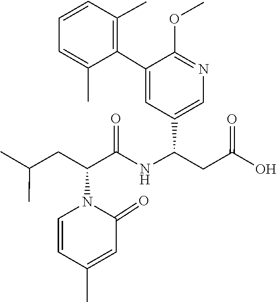 [SMILES: COc1ncc([C@H](CC(=O)O)NC(=O)[C@@H](CC(C)C)n2ccc(C)cc2=O)cc1-c1c(C)cccc1C] |
| 16 | 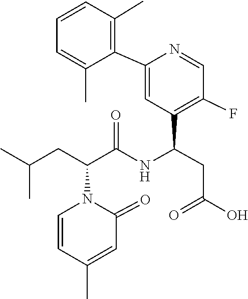 [SMILES: Cc1ccn([C@H](CC(C)C)C(=O)N[C@H](CC(=O)O)c2cc(-c3c(C)cccc3C)ncc2F)c(=O)c1] | 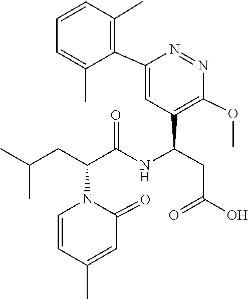 [SMILES: COc1nnc(-c2c(C)cccc2C)cc1[C@@H](CC(=O)O)NC(=O)[C@@H](CC(C)C)n1ccc(C)cc1=O] | 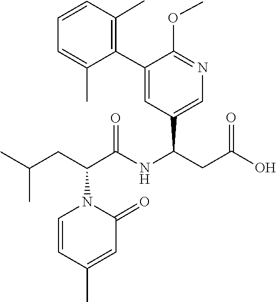 [SMILES: COc1ncc([C@@H](CC(=O)O)NC(=O)[C@@H](CC(C)C)n2ccc(C)cc2=O)cc1-c1c(C)cccc1C] |
| 17 | 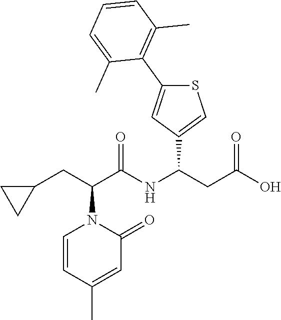 [SMILES: Cc1ccn([C@@H](CC2CC2)C(=O)N[C@@H](CC(=O)O)c2csc(-c3c(C)cccc3C)c2)c(=O)c1] | 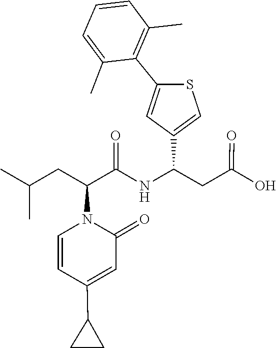 [SMILES: Cc1cccc(C)c1-c1cc([C@H](CC(=O)O)NC(=O)[C@H](CC(C)C)n2ccc(C3CC3)cc2=O)cs1] | 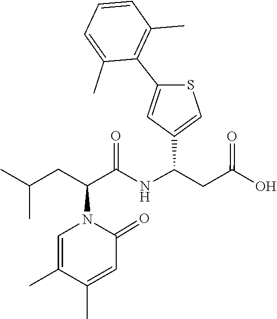 [SMILES: Cc1cc(=O)n([C@@H](CC(C)C)C(=O)N[C@@H](CC(=O)O)c2csc(-c3c(C)cccc3C)c2)cc1C] |
| 18 | 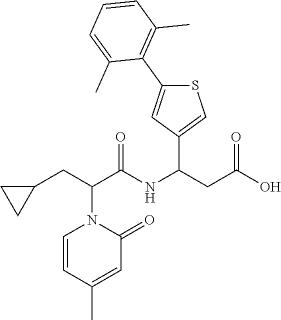 [SMILES: Cc1ccn(C(CC2CC2)C(=O)NC(CC(=O)O)c2csc(-c3c(C)cccc3C)c2)c(=O)c1] | 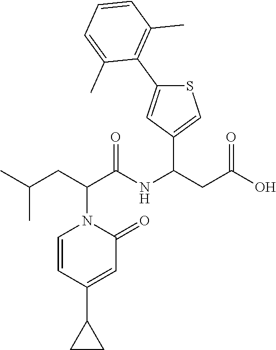 [SMILES: Cc1cccc(C)c1-c1cc(C(CC(=O)O)NC(=O)C(CC(C)C)n2ccc(C3CC3)cc2=O)cs1] | 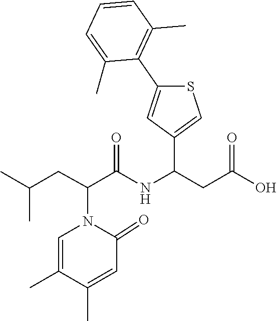 [SMILES: Cc1cc(=O)n(C(CC(C)C)C(=O)NC(CC(=O)O)c2csc(-c3c(C)cccc3C)c2)cc1C] |
| 19 | 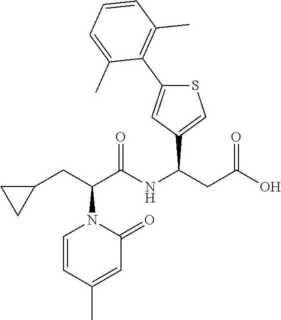 [SMILES: Cc1ccn([C@@H](CC2CC2)C(=O)N[C@H](CC(=O)O)c2csc(-c3c(C)cccc3C)c2)c(=O)c1] | 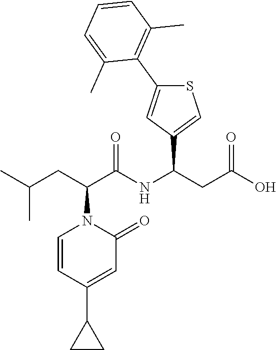 [SMILES: Cc1cccc(C)c1-c1cc([C@@H](CC(=O)O)NC(=O)[C@H](CC(C)C)n2ccc(C3CC3)cc2=O)cs1] | 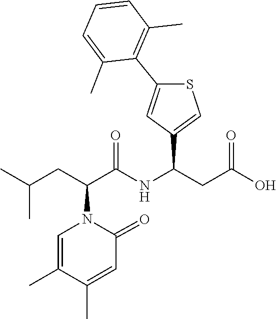 [SMILES: Cc1cc(=O)n([C@@H](CC(C)C)C(=O)N[C@H](CC(=O)O)c2csc(-c3c(C)cccc3C)c2)cc1C] |
| 20 | 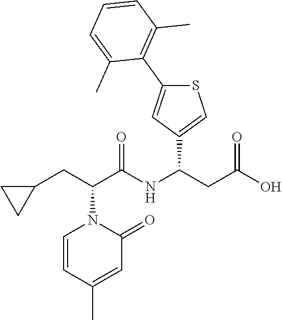 [SMILES: Cc1ccn([C@H](CC2CC2)C(=O)N[C@@H](CC(=O)O)c2csc(-c3c(C)cccc3C)c2)c(=O)c1] | 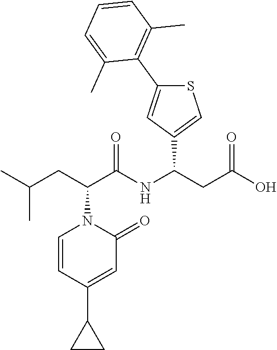 [SMILES: Cc1cccc(C)c1-c1cc([C@H](CC(=O)O)NC(=O)[C@@H](CC(C)C)n2ccc(C3CC3)cc2=O)cs1] | 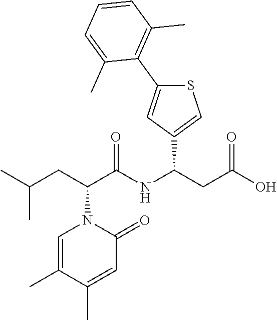 [SMILES: Cc1cc(=O)n([C@H](CC(C)C)C(=O)N[C@@H](CC(=O)O)c2csc(-c3c(C)cccc3C)c2)cc1C] |
| 21 | 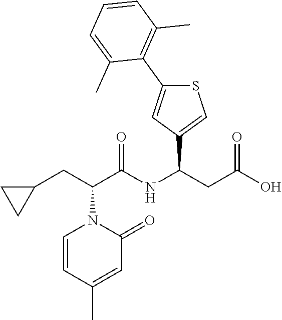 [SMILES: Cc1ccn([C@H](CC2CC2)C(=O)N[C@H](CC(=O)O)c2csc(-c3c(C)cccc3C)c2)c(=O)c1] | 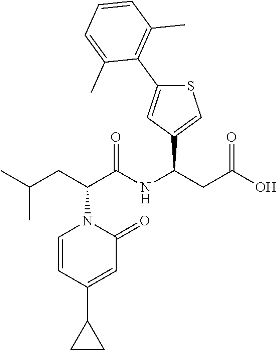 [SMILES: Cc1cccc(C)c1-c1cc([C@@H](CC(=O)O)NC(=O)[C@@H](CC(C)C)n2ccc(C3CC3)cc2=O)cs1] | 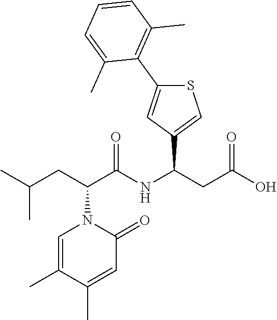 [SMILES: Cc1cc(=O)n([C@H](CC(C)C)C(=O)N[C@H](CC(=O)O)c2csc(-c3c(C)cccc3C)c2)cc1C] |
| 22 | 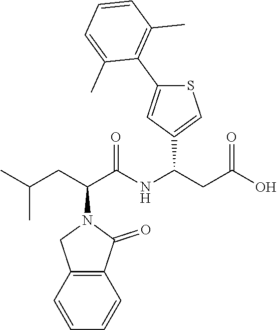 [SMILES: Cc1cccc(C)c1-c1cc([C@H](CC(=O)O)NC(=O)[C@H](CC(C)C)N2Cc3ccccc3C2=O)cs1] | 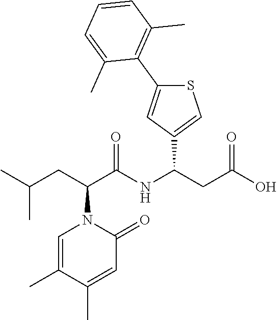 [SMILES: Cc1cc(=O)n([C@@H](CC(C)C)C(=O)N[C@@H](CC(=O)O)c2csc(-c3c(C)cccc3C)c2)cc1C] | 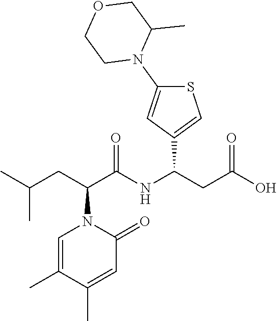 [SMILES: Cc1cc(=O)n([C@@H](CC(C)C)C(=O)N[C@@H](CC(=O)O)c2csc(N3CCOCC3C)c2)cc1C] |
| 23 | 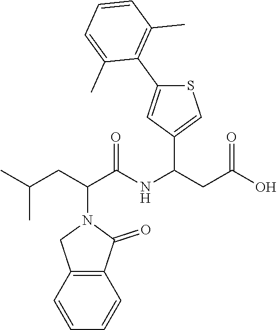 [SMILES: Cc1cccc(C)c1-c1cc(C(CC(=O)O)NC(=O)C(CC(C)C)N2Cc3ccccc3C2=O)cs1] | 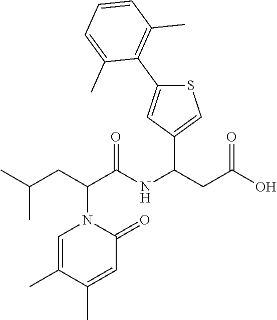 [SMILES: Cc1cc(=O)n(C(CC(C)C)C(=O)NC(CC(=O)O)c2csc(-c3c(C)cccc3C)c2)cc1C] | 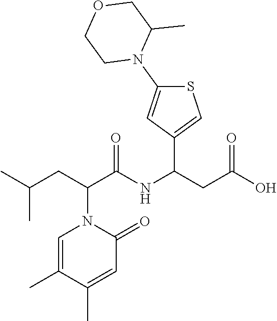 [SMILES: Cc1cc(=O)n(C(CC(C)C)C(=O)NC(CC(=O)O)c2csc(N3CCOCC3C)c2)cc1C] |
| 24 | 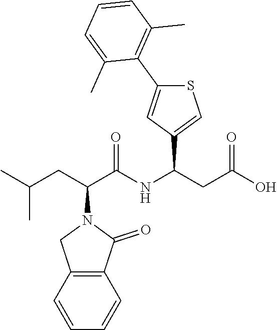 [SMILES: Cc1cccc(C)c1-c1cc([C@@H](CC(=O)O)NC(=O)[C@H](CC(C)C)N2Cc3ccccc3C2=O)cs1] | 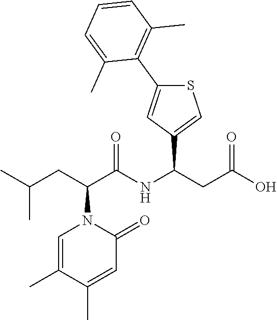 [SMILES: Cc1cc(=O)n([C@@H](CC(C)C)C(=O)N[C@H](CC(=O)O)c2csc(-c3c(C)cccc3C)c2)cc1C] | 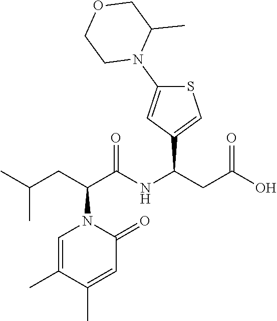 [SMILES: Cc1cc(=O)n([C@@H](CC(C)C)C(=O)N[C@H](CC(=O)O)c2csc(N3CCOCC3C)c2)cc1C] |
| 25 | 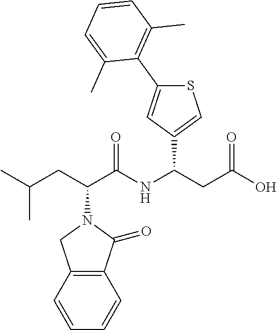 [SMILES: Cc1cccc(C)c1-c1cc([C@H](CC(=O)O)NC(=O)[C@@H](CC(C)C)N2Cc3ccccc3C2=O)cs1] | 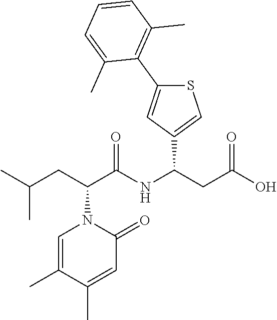 [SMILES: Cc1cc(=O)n([C@H](CC(C)C)C(=O)N[C@@H](CC(=O)O)c2csc(-c3c(C)cccc3C)c2)cc1C] | 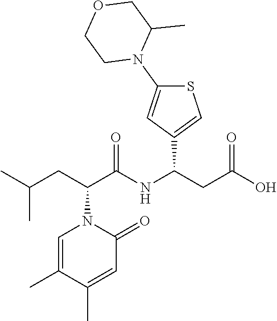 [SMILES: Cc1cc(=O)n([C@H](CC(C)C)C(=O)N[C@@H](CC(=O)O)c2csc(N3CCOCC3C)c2)cc1C] |
| 26 | 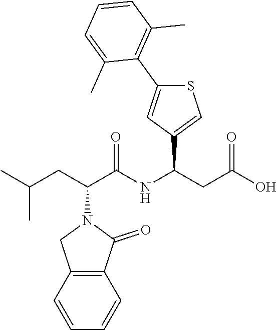 [SMILES: Cc1cccc(C)c1-c1cc([C@@H](CC(=O)O)NC(=O)[C@@H](CC(C)C)N2Cc3ccccc3C2=O)cs1] | 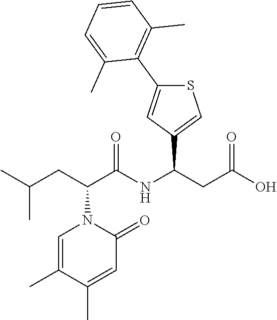 [SMILES: Cc1cc(=O)n([C@H](CC(C)C)C(=O)N[C@H](CC(=O)O)c2csc(-c3c(C)cccc3C)c2)cc1C] | 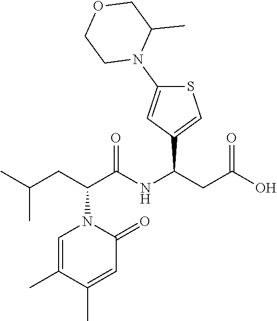 [SMILES: Cc1cc(=O)n([C@H](CC(C)C)C(=O)N[C@H](CC(=O)O)c2csc(N3CCOCC3C)c2)cc1C] |
| 27 | 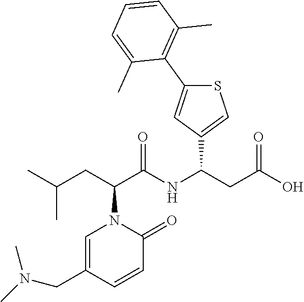 [SMILES: Cc1cccc(C)c1-c1cc([C@H](CC(=O)O)NC(=O)[C@H](CC(C)C)n2cc(CN(C)C)ccc2=O)cs1] | 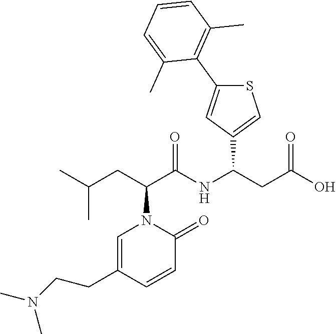 [SMILES: Cc1cccc(C)c1-c1cc([C@H](CC(=O)O)NC(=O)[C@H](CC(C)C)n2cc(CCN(C)C)ccc2=O)cs1] | 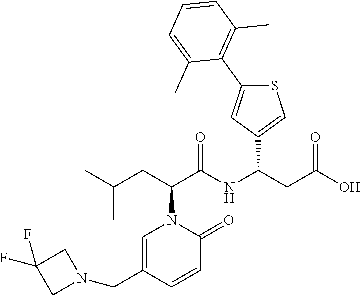 [SMILES: Cc1cccc(C)c1-c1cc([C@H](CC(=O)O)NC(=O)[C@H](CC(C)C)n2cc(CN3CC(F)(F)C3)ccc2=O)cs1] |
| 28 | 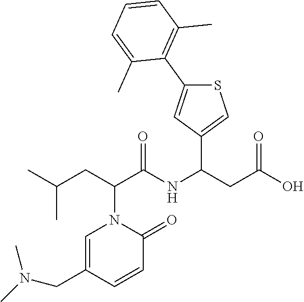 [SMILES: Cc1cccc(C)c1-c1cc(C(CC(=O)O)NC(=O)C(CC(C)C)n2cc(CN(C)C)ccc2=O)cs1] | 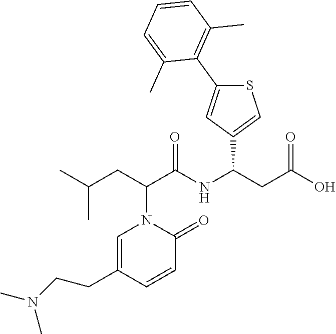 [SMILES: Cc1cccc(C)c1-c1cc([C@H](CC(=O)O)NC(=O)C(CC(C)C)n2cc(CCN(C)C)ccc2=O)cs1] | 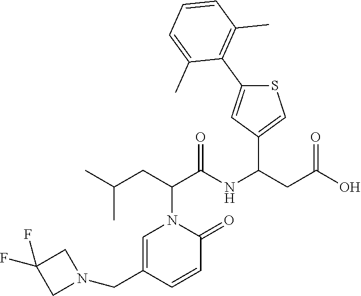 [SMILES: Cc1cccc(C)c1-c1cc(C(CC(=O)O)NC(=O)C(CC(C)C)n2cc(CN3CC(F)(F)C3)ccc2=O)cs1] |
| 29 | 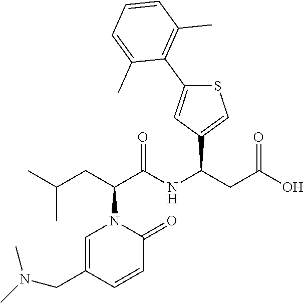 [SMILES: Cc1cccc(C)c1-c1cc([C@@H](CC(=O)O)NC(=O)[C@H](CC(C)C)n2cc(CN(C)C)ccc2=O)cs1] | 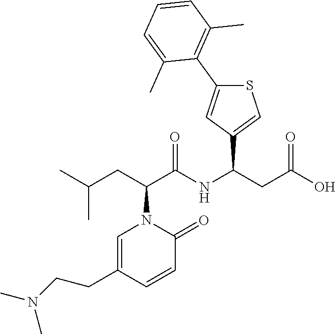 [SMILES: Cc1cccc(C)c1-c1cc([C@@H](CC(=O)O)NC(=O)[C@H](CC(C)C)n2cc(CCN(C)C)ccc2=O)cs1] | 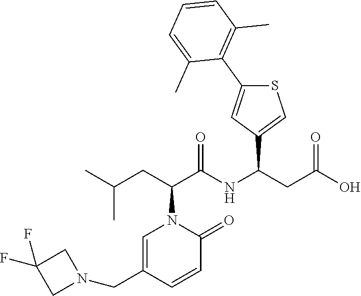 [SMILES: Cc1cccc(C)c1-c1cc([C@@H](CC(=O)O)NC(=O)[C@H](CC(C)C)n2cc(CN3CC(F)(F)C3)ccc2=O)cs1] |
| 30 | 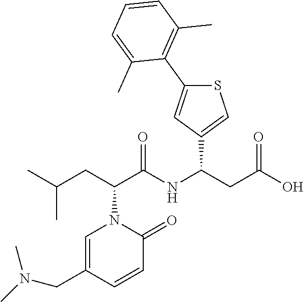 [SMILES: Cc1cccc(C)c1-c1cc([C@H](CC(=O)O)NC(=O)[C@@H](CC(C)C)n2cc(CN(C)C)ccc2=O)cs1] | 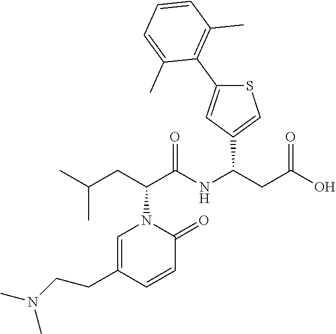 [SMILES: Cc1cccc(C)c1-c1cc([C@H](CC(=O)O)NC(=O)[C@@H](CC(C)C)n2cc(CCN(C)C)ccc2=O)cs1] | 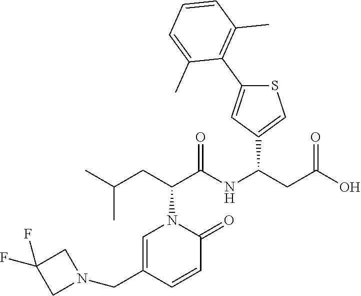 [SMILES: Cc1cccc(C)c1-c1cc([C@H](CC(=O)O)NC(=O)[C@@H](CC(C)C)n2cc(CN3CC(F)(F)C3)ccc2=O)cs1] |
| 31 | 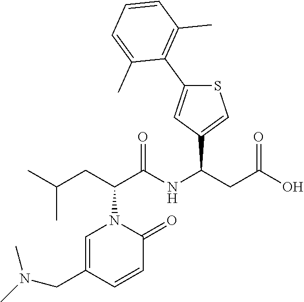 [SMILES: Cc1cccc(C)c1-c1cc([C@@H](CC(=O)O)NC(=O)[C@@H](CC(C)C)n2cc(CN(C)C)ccc2=O)cs1] | 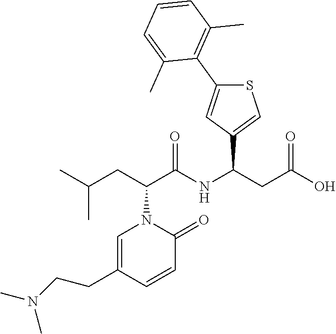 [SMILES: Cc1cccc(C)c1-c1cc([C@@H](CC(=O)O)NC(=O)[C@@H](CC(C)C)n2cc(CCN(C)C)ccc2=O)cs1] | 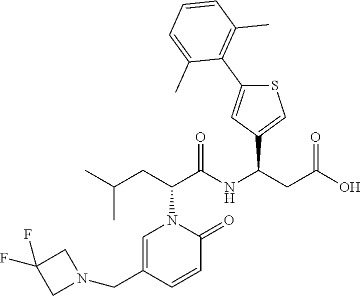 [SMILES: Cc1cccc(C)c1-c1cc([C@@H](CC(=O)O)NC(=O)[C@@H](CC(C)C)n2cc(CN3CC(F)(F)C3)ccc2=O)cs1] |
| 32 | 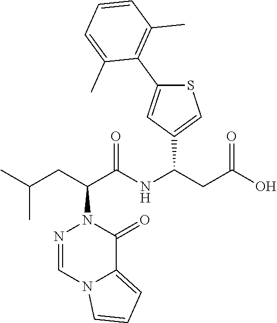 [SMILES: Cc1cccc(C)c1-c1cc([C@H](CC(=O)O)NC(=O)[C@H](CC(C)C)n2ncn3cccc3c2=O)cs1] | 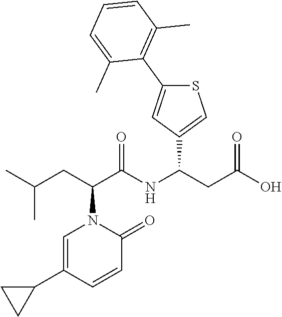 [SMILES: Cc1cccc(C)c1-c1cc([C@H](CC(=O)O)NC(=O)[C@H](CC(C)C)n2cc(C3CC3)ccc2=O)cs1] | 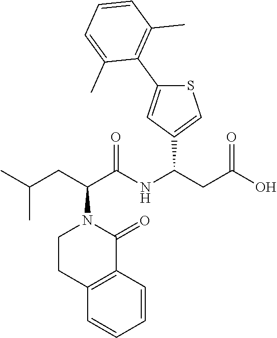 [SMILES: Cc1cccc(C)c1-c1cc([C@H](CC(=O)O)NC(=O)[C@H](CC(C)C)N2CCc3ccccc3C2=O)cs1] |
| 33 | 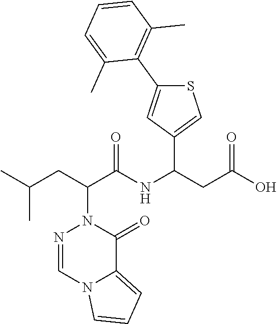 [SMILES: Cc1cccc(C)c1-c1cc(C(CC(=O)O)NC(=O)C(CC(C)C)n2ncn3cccc3c2=O)cs1] | 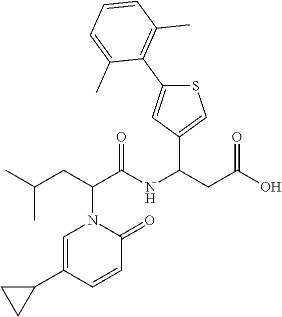 [SMILES: Cc1cccc(C)c1-c1cc(C(CC(=O)O)NC(=O)C(CC(C)C)n2cc(C3CC3)ccc2=O)cs1] | 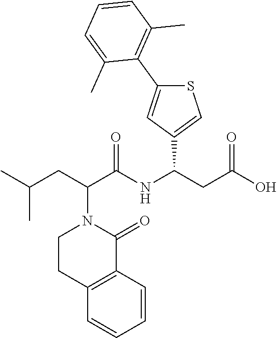 [SMILES: Cc1cccc(C)c1-c1cc([C@H](CC(=O)O)NC(=O)C(CC(C)C)N2CCc3ccccc3C2=O)cs1] |
| 34 | 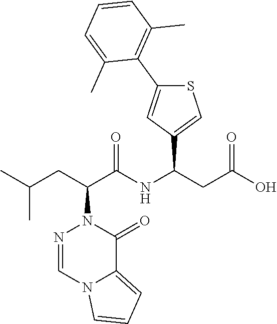 [SMILES: Cc1cccc(C)c1-c1cc([C@@H](CC(=O)O)NC(=O)[C@H](CC(C)C)n2ncn3cccc3c2=O)cs1] | 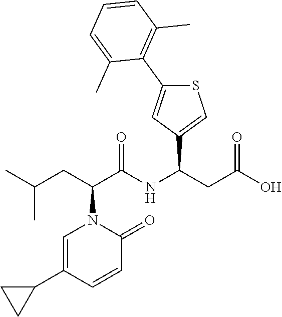 [SMILES: Cc1cccc(C)c1-c1cc([C@@H](CC(=O)O)NC(=O)[C@H](CC(C)C)n2cc(C3CC3)ccc2=O)cs1] | 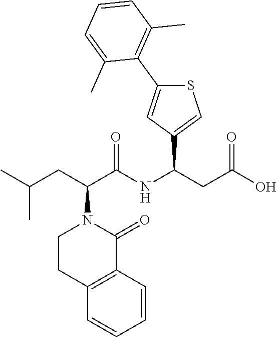 [SMILES: Cc1cccc(C)c1-c1cc([C@@H](CC(=O)O)NC(=O)[C@H](CC(C)C)N2CCc3ccccc3C2=O)cs1] |
| 35 | 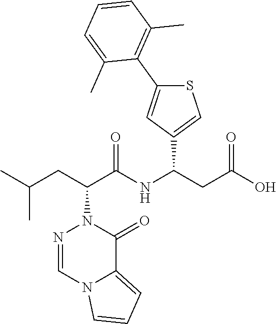 [SMILES: Cc1cccc(C)c1-c1cc([C@H](CC(=O)O)NC(=O)[C@@H](CC(C)C)n2ncn3cccc3c2=O)cs1] | 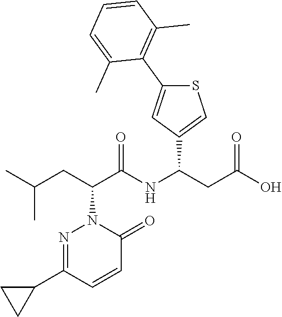 [SMILES: Cc1cccc(C)c1-c1cc([C@H](CC(=O)O)NC(=O)[C@@H](CC(C)C)n2nc(C3CC3)ccc2=O)cs1] | 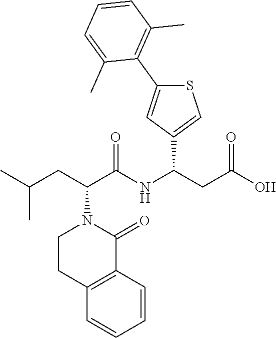 [SMILES: Cc1cccc(C)c1-c1cc([C@H](CC(=O)O)NC(=O)[C@@H](CC(C)C)N2CCc3ccccc3C2=O)cs1] |
| 36 | 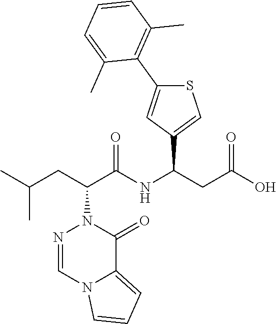 [SMILES: Cc1cccc(C)c1-c1cc([C@@H](CC(=O)O)NC(=O)[C@@H](CC(C)C)n2ncn3cccc3c2=O)cs1] | 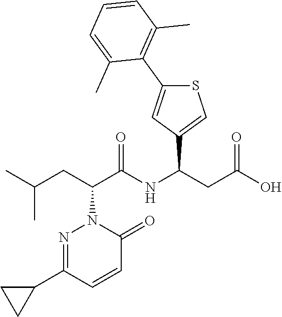 [SMILES: Cc1cccc(C)c1-c1cc([C@@H](CC(=O)O)NC(=O)[C@@H](CC(C)C)n2nc(C3CC3)ccc2=O)cs1] | 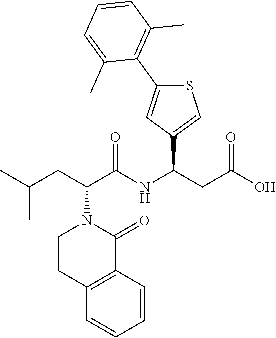 [SMILES: Cc1cccc(C)c1-c1cc([C@@H](CC(=O)O)NC(=O)[C@@H](CC(C)C)N2CCc3ccccc3C2=O)cs1] |
| 37 | 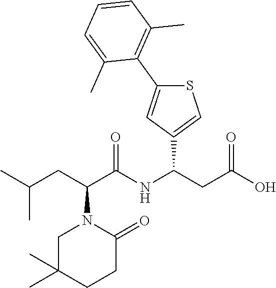 [SMILES: Cc1cccc(C)c1-c1cc([C@H](CC(=O)O)NC(=O)[C@H](CC(C)C)N2CC(C)(C)CCC2=O)cs1] | 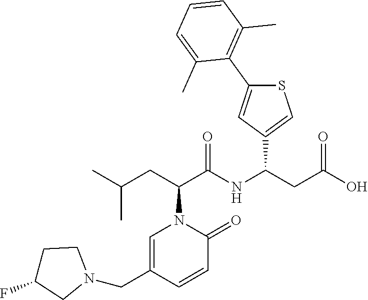 [SMILES: Cc1cccc(C)c1-c1cc([C@H](CC(=O)O)NC(=O)[C@H](CC(C)C)n2cc(CN3CC[C@@H](F)C3)ccc2=O)cs1] | 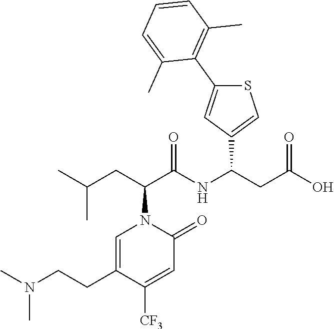 [SMILES: Cc1cccc(C)c1-c1cc([C@H](CC(=O)O)NC(=O)[C@H](CC(C)C)n2cc(CCN(C)C)c(C(F)(F)F)cc2=O)cs1] |
| 38 | 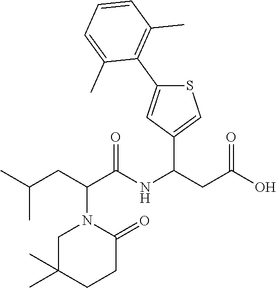 [SMILES: Cc1cccc(C)c1-c1cc(C(CC(=O)O)NC(=O)C(CC(C)C)N2CC(C)(C)CCC2=O)cs1] | 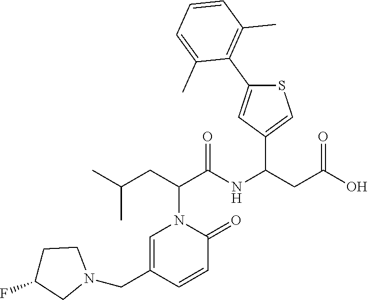 [SMILES: Cc1cccc(C)c1-c1cc(C(CC(=O)O)NC(=O)C(CC(C)C)n2cc(CN3CC[C@@H](F)C3)ccc2=O)cs1] | 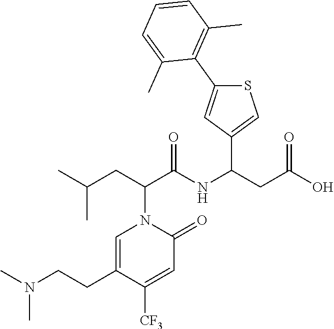 [SMILES: Cc1cccc(C)c1-c1cc(C(CC(=O)O)NC(=O)C(CC(C)C)n2cc(CCN(C)C)c(C(F)(F)F)cc2=O)cs1] |
| 39 | 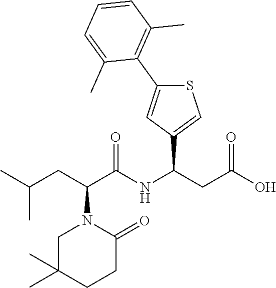 [SMILES: Cc1cccc(C)c1-c1cc([C@@H](CC(=O)O)NC(=O)[C@H](CC(C)C)N2CC(C)(C)CCC2=O)cs1] | 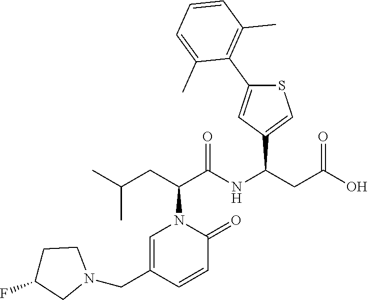 [SMILES: Cc1cccc(C)c1-c1cc([C@@H](CC(=O)O)NC(=O)[C@H](CC(C)C)n2cc(CN3CC[C@@H](F)C3)ccc2=O)cs1] | 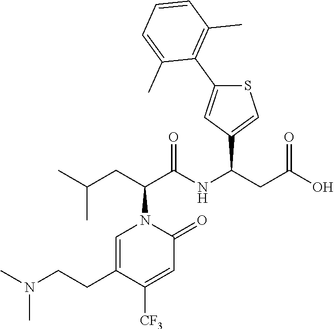 [SMILES: Cc1cccc(C)c1-c1cc([C@@H](CC(=O)O)NC(=O)[C@H](CC(C)C)n2cc(CCN(C)C)c(C(F)(F)F)cc2=O)cs1] |
| 40 | 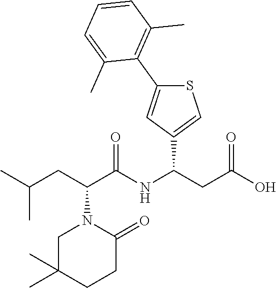 [SMILES: Cc1cccc(C)c1-c1cc([C@H](CC(=O)O)NC(=O)[C@@H](CC(C)C)N2CC(C)(C)CCC2=O)cs1] | 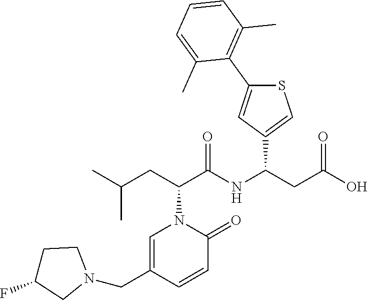 [SMILES: Cc1cccc(C)c1-c1cc([C@H](CC(=O)O)NC(=O)[C@@H](CC(C)C)n2cc(CN3CC[C@@H](F)C3)ccc2=O)cs1] | 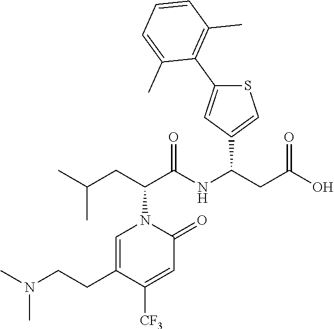 [SMILES: Cc1cccc(C)c1-c1cc([C@H](CC(=O)O)NC(=O)[C@@H](CC(C)C)n2cc(CCN(C)C)c(C(F)(F)F)cc2=O)cs1] |
| 41 | 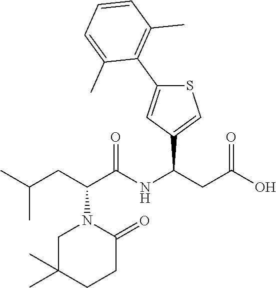 [SMILES: Cc1cccc(C)c1-c1cc([C@@H](CC(=O)O)NC(=O)[C@@H](CC(C)C)N2CC(C)(C)CCC2=O)cs1] | 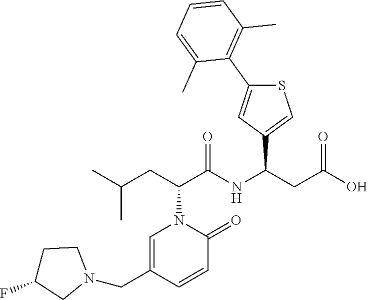 [SMILES: Cc1cccc(C)c1-c1cc([C@@H](CC(=O)O)NC(=O)[C@@H](CC(C)C)n2cc(CN3CC[C@@H](F)C3)ccc2=O)cs1] | 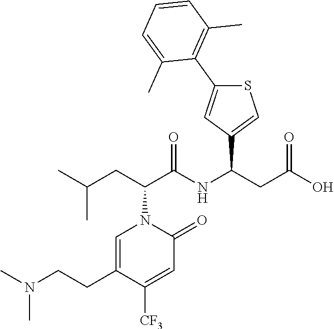 [SMILES: Cc1cccc(C)c1-c1cc([C@@H](CC(=O)O)NC(=O)[C@@H](CC(C)C)n2cc(CCN(C)C)c(C(F)(F)F)cc2=O)cs1] |
| 42 | 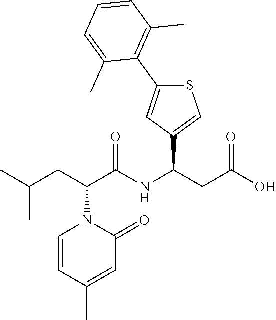 [SMILES: Cc1ccn([C@H](CC(C)C)C(=O)N[C@H](CC(=O)O)c2csc(-c3c(C)cccc3C)c2)c(=O)c1] | 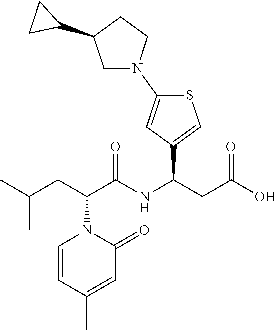 [SMILES: Cc1ccn([C@H](CC(C)C)C(=O)N[C@H](CC(=O)O)c2csc(N3CC[C@H](C4CC4)C3)c2)c(=O)c1] | 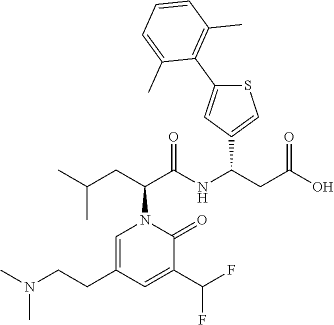 [SMILES: Cc1cccc(C)c1-c1cc([C@H](CC(=O)O)NC(=O)[C@H](CC(C)C)n2cc(CCN(C)C)cc(C(F)F)c2=O)cs1] |
| 43 | 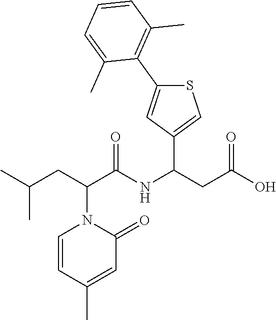 [SMILES: Cc1ccn(C(CC(C)C)C(=O)NC(CC(=O)O)c2csc(-c3c(C)cccc3C)c2)c(=O)c1] | 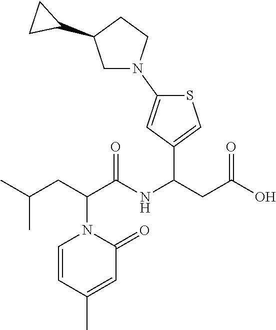 [SMILES: Cc1ccn(C(CC(C)C)C(=O)NC(CC(=O)O)c2csc(N3CC[C@H](C4CC4)C3)c2)c(=O)c1] | 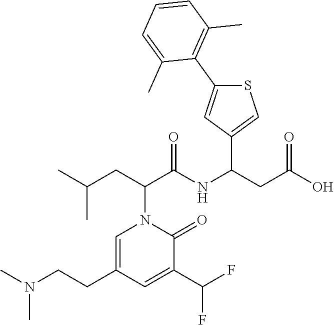 [SMILES: Cc1cccc(C)c1-c1cc(C(CC(=O)O)NC(=O)C(CC(C)C)n2cc(CCN(C)C)cc(C(F)F)c2=O)cs1] |
| 44 | 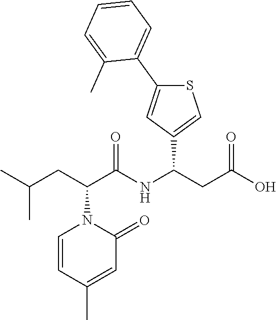 [SMILES: Cc1ccn([C@H](CC(C)C)C(=O)N[C@@H](CC(=O)O)c2csc(-c3ccccc3C)c2)c(=O)c1] | 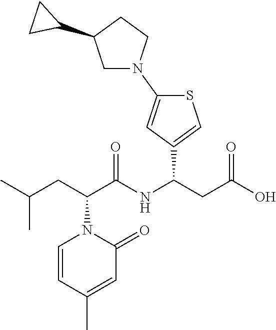 [SMILES: Cc1ccn([C@H](CC(C)C)C(=O)N[C@@H](CC(=O)O)c2csc(N3CC[C@H](C4CC4)C3)c2)c(=O)c1] | 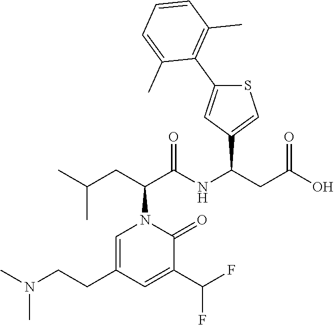 [SMILES: Cc1cccc(C)c1-c1cc([C@@H](CC(=O)O)NC(=O)[C@H](CC(C)C)n2cc(CCN(C)C)cc(C(F)F)c2=O)cs1] |
| 45 | 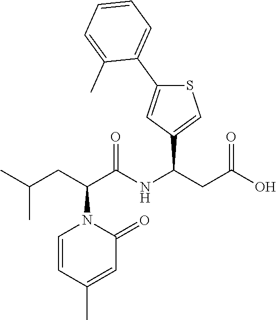 [SMILES: Cc1ccn([C@@H](CC(C)C)C(=O)N[C@H](CC(=O)O)c2csc(-c3ccccc3C)c2)c(=O)c1] | 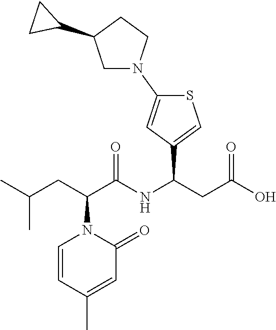 [SMILES: Cc1ccn([C@@H](CC(C)C)C(=O)N[C@H](CC(=O)O)c2csc(N3CC[C@H](C4CC4)C3)c2)c(=O)c1] | 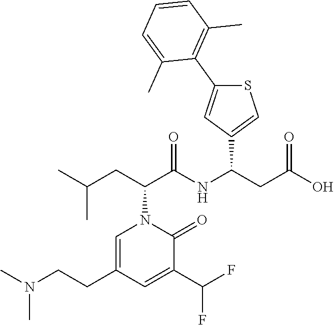 [SMILES: Cc1cccc(C)c1-c1cc([C@H](CC(=O)O)NC(=O)[C@@H](CC(C)C)n2cc(CCN(C)C)cc(C(F)F)c2=O)cs1] |
| 46 | 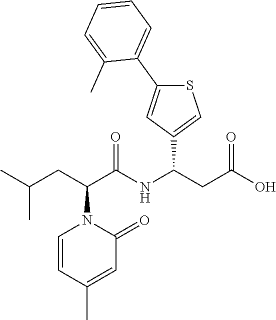 [SMILES: Cc1ccn([C@@H](CC(C)C)C(=O)N[C@@H](CC(=O)O)c2csc(-c3ccccc3C)c2)c(=O)c1] | 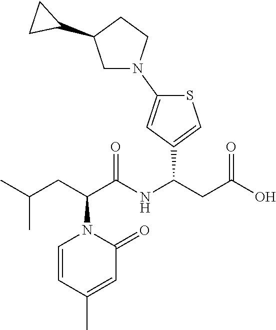 [SMILES: Cc1ccn([C@@H](CC(C)C)C(=O)N[C@@H](CC(=O)O)c2csc(N3CC[C@H](C4CC4)C3)c2)c(=O)c1] | 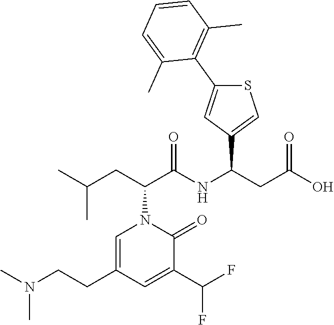 [SMILES: Cc1cccc(C)c1-c1cc([C@@H](CC(=O)O)NC(=O)[C@@H](CC(C)C)n2cc(CCN(C)C)cc(C(F)F)c2=O)cs1] |
| 47 | 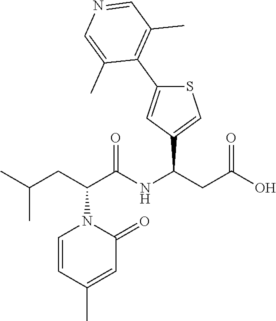 [SMILES: Cc1ccn([C@H](CC(C)C)C(=O)N[C@H](CC(=O)O)c2csc(-c3c(C)cncc3C)c2)c(=O)c1] | 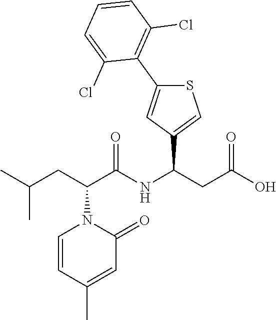 [SMILES: Cc1ccn([C@H](CC(C)C)C(=O)N[C@H](CC(=O)O)c2csc(-c3c(Cl)cccc3Cl)c2)c(=O)c1] | 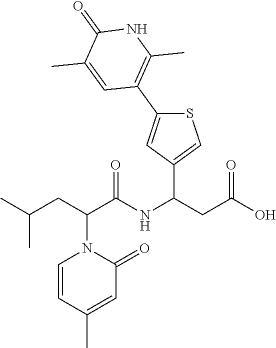 [SMILES: Cc1ccn(C(CC(C)C)C(=O)NC(CC(=O)O)c2csc(-c3cc(C)c(=O)[nH]c3C)c2)c(=O)c1] |
| 48 | 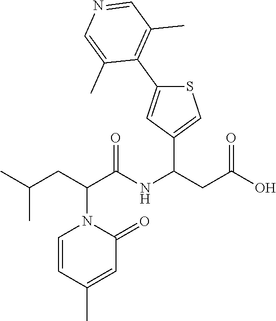 [SMILES: Cc1ccn(C(CC(C)C)C(=O)NC(CC(=O)O)c2csc(-c3c(C)cncc3C)c2)c(=O)c1] | 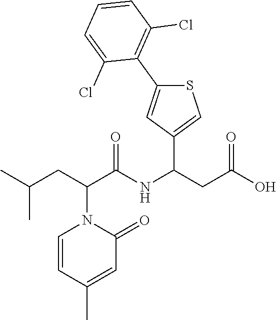 [SMILES: Cc1ccn(C(CC(C)C)C(=O)NC(CC(=O)O)c2csc(-c3c(Cl)cccc3Cl)c2)c(=O)c1] | 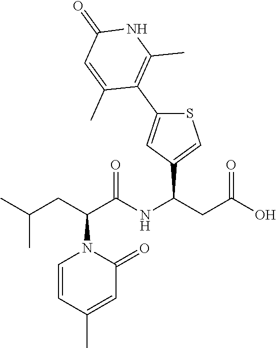 [SMILES: Cc1ccn([C@@H](CC(C)C)C(=O)N[C@H](CC(=O)O)c2csc(-c3c(C)cc(=O)[nH]c3C)c2)c(=O)c1] |
| 49 | 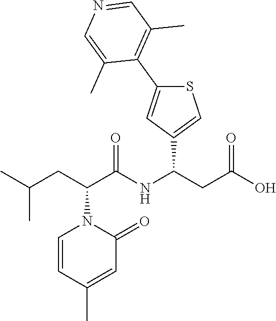 [SMILES: Cc1ccn([C@H](CC(C)C)C(=O)N[C@@H](CC(=O)O)c2csc(-c3c(C)cncc3C)c2)c(=O)c1] | 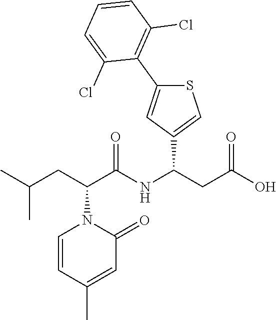 [SMILES: Cc1ccn([C@H](CC(C)C)C(=O)N[C@@H](CC(=O)O)c2csc(-c3c(Cl)cccc3Cl)c2)c(=O)c1] | 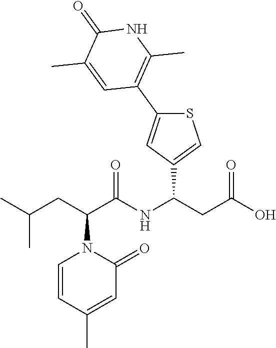 [SMILES: Cc1ccn([C@@H](CC(C)C)C(=O)N[C@@H](CC(=O)O)c2csc(-c3cc(C)c(=O)[nH]c3C)c2)c(=O)c1] |
| 50 | 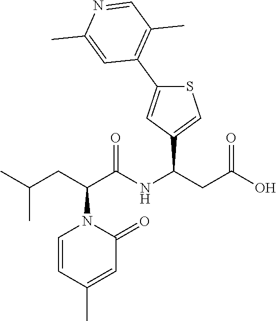 [SMILES: Cc1ccn([C@@H](CC(C)C)C(=O)N[C@H](CC(=O)O)c2csc(-c3cc(C)ncc3C)c2)c(=O)c1] | 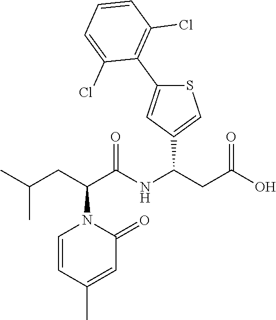 [SMILES: Cc1ccn([C@@H](CC(C)C)C(=O)N[C@@H](CC(=O)O)c2csc(-c3c(Cl)cccc3Cl)c2)c(=O)c1] | 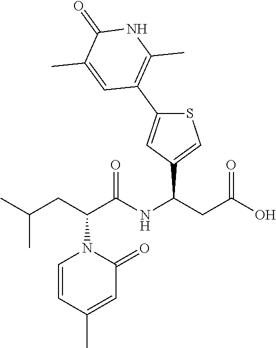 [SMILES: Cc1ccn([C@H](CC(C)C)C(=O)N[C@H](CC(=O)O)c2csc(-c3cc(C)c(=O)[nH]c3C)c2)c(=O)c1] |
| 51 | 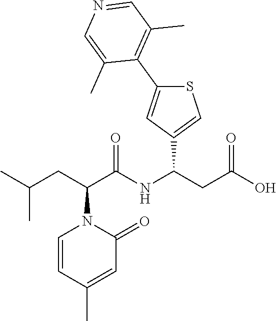 [SMILES: Cc1ccn([C@@H](CC(C)C)C(=O)N[C@@H](CC(=O)O)c2csc(-c3c(C)cncc3C)c2)c(=O)c1] | 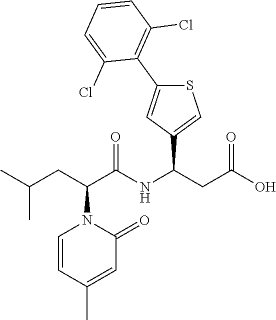 [SMILES: Cc1ccn([C@@H](CC(C)C)C(=O)N[C@H](CC(=O)O)c2csc(-c3c(Cl)cccc3Cl)c2)c(=O)c1] | 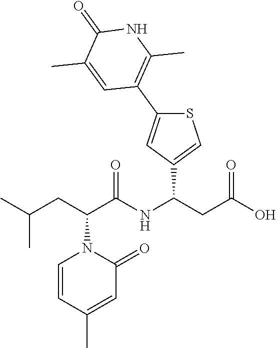 [SMILES: Cc1ccn([C@H](CC(C)C)C(=O)N[C@@H](CC(=O)O)c2csc(-c3cc(C)c(=O)[nH]c3C)c2)c(=O)c1] |
| 52 | 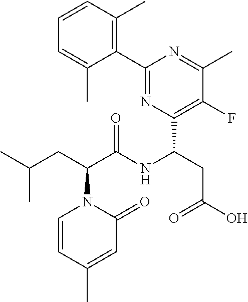 [SMILES: Cc1ccn([C@@H](CC(C)C)C(=O)N[C@@H](CC(=O)O)c2nc(-c3c(C)cccc3C)nc(C)c2F)c(=O)c1] | 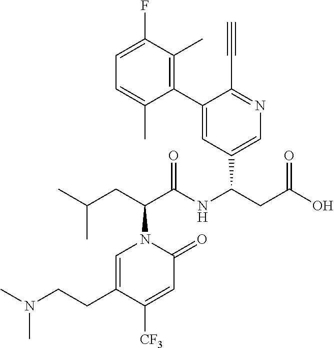 [SMILES: C#Cc1ncc([C@H](CC(=O)O)NC(=O)[C@H](CC(C)C)n2cc(CCN(C)C)c(C(F)(F)F)cc2=O)cc1-c1c(C)ccc(F)c1C] | 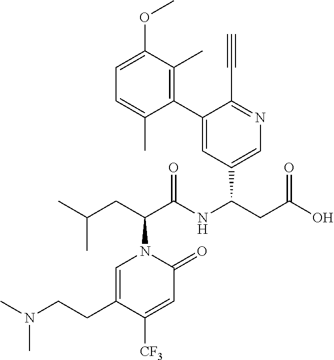 [SMILES: C#Cc1ncc([C@H](CC(=O)O)NC(=O)[C@H](CC(C)C)n2cc(CCN(C)C)c(C(F)(F)F)cc2=O)cc1-c1c(C)ccc(OC)c1C] |
| 53 | 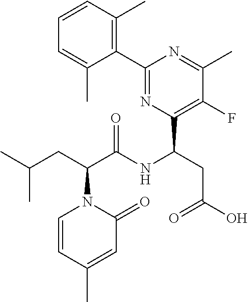 [SMILES: Cc1ccn([C@@H](CC(C)C)C(=O)N[C@H](CC(=O)O)c2nc(-c3c(C)cccc3C)nc(C)c2F)c(=O)c1] | 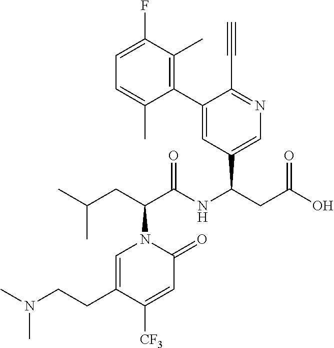 [SMILES: C#Cc1ncc([C@@H](CC(=O)O)NC(=O)[C@H](CC(C)C)n2cc(CCN(C)C)c(C(F)(F)F)cc2=O)cc1-c1c(C)ccc(F)c1C] | 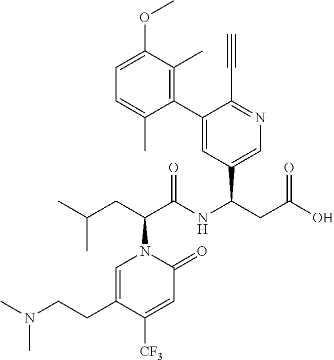 [SMILES: C#Cc1ncc([C@@H](CC(=O)O)NC(=O)[C@H](CC(C)C)n2cc(CCN(C)C)c(C(F)(F)F)cc2=O)cc1-c1c(C)ccc(OC)c1C] |
| 54 | 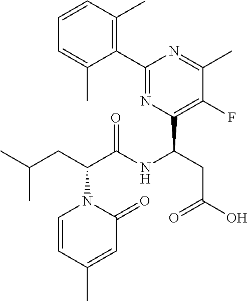 [SMILES: Cc1ccn([C@H](CC(C)C)C(=O)N[C@H](CC(=O)O)c2nc(-c3c(C)cccc3C)nc(C)c2F)c(=O)c1] | 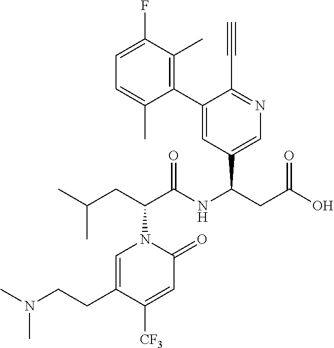 [SMILES: C#Cc1ncc([C@@H](CC(=O)O)NC(=O)[C@@H](CC(C)C)n2cc(CCN(C)C)c(C(F)(F)F)cc2=O)cc1-c1c(C)ccc(F)c1C] | 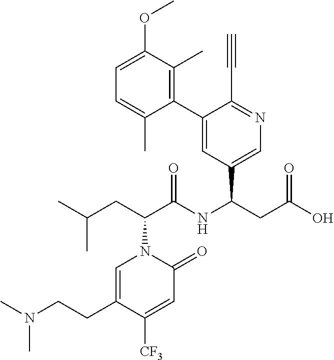 [SMILES: C#Cc1ncc([C@@H](CC(=O)O)NC(=O)[C@@H](CC(C)C)n2cc(CCN(C)C)c(C(F)(F)F)cc2=O)cc1-c1c(C)ccc(OC)c1C] |
| 55 | 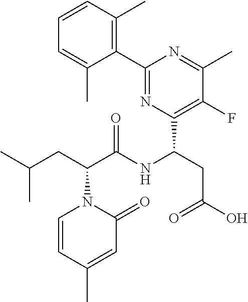 [SMILES: Cc1ccn([C@H](CC(C)C)C(=O)N[C@@H](CC(=O)O)c2nc(-c3c(C)cccc3C)nc(C)c2F)c(=O)c1] | 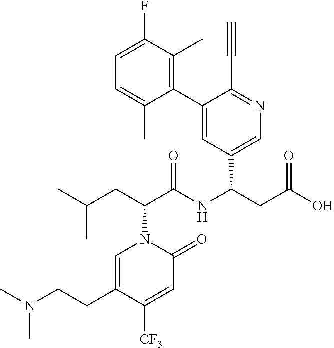 [SMILES: C#Cc1ncc([C@H](CC(=O)O)NC(=O)[C@@H](CC(C)C)n2cc(CCN(C)C)c(C(F)(F)F)cc2=O)cc1-c1c(C)ccc(F)c1C] | 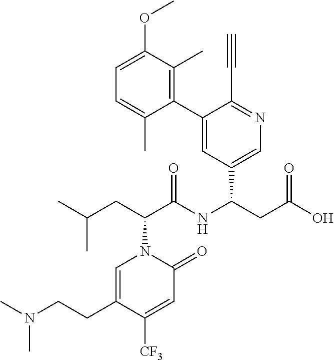 [SMILES: C#Cc1ncc([C@H](CC(=O)O)NC(=O)[C@@H](CC(C)C)n2cc(CCN(C)C)c(C(F)(F)F)cc2=O)cc1-c1c(C)ccc(OC)c1C] |
| 56 | 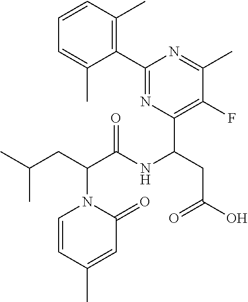 [SMILES: Cc1ccn(C(CC(C)C)C(=O)NC(CC(=O)O)c2nc(-c3c(C)cccc3C)nc(C)c2F)c(=O)c1] | 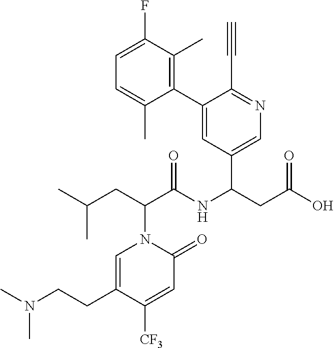 [SMILES: C#Cc1ncc(C(CC(=O)O)NC(=O)C(CC(C)C)n2cc(CCN(C)C)c(C(F)(F)F)cc2=O)cc1-c1c(C)ccc(F)c1C] | 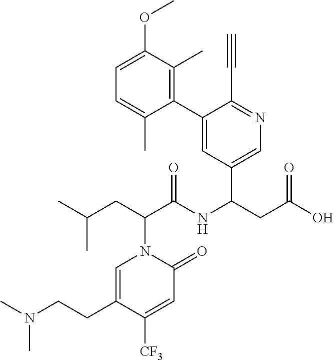 [SMILES: C#Cc1ncc(C(CC(=O)O)NC(=O)C(CC(C)C)n2cc(CCN(C)C)c(C(F)(F)F)cc2=O)cc1-c1c(C)ccc(OC)c1C] |
| 57 | 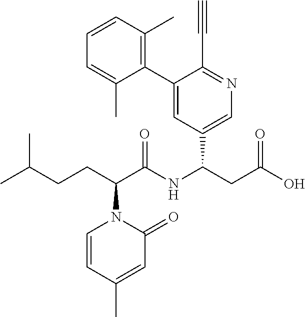 [SMILES: C#Cc1ncc([C@H](CC(=O)O)NC(=O)[C@H](CCC(C)C)n2ccc(C)cc2=O)cc1-c1c(C)cccc1C] | 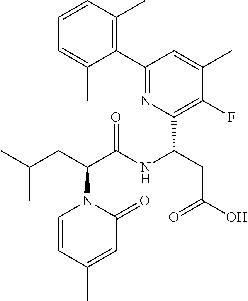 [SMILES: Cc1ccn([C@@H](CC(C)C)C(=O)N[C@@H](CC(=O)O)c2nc(-c3c(C)cccc3C)cc(C)c2F)c(=O)c1] | 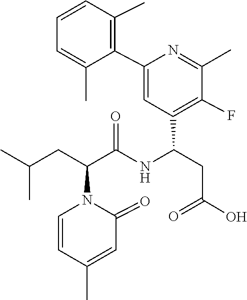 [SMILES: Cc1ccn([C@@H](CC(C)C)C(=O)N[C@@H](CC(=O)O)c2cc(-c3c(C)cccc3C)nc(C)c2F)c(=O)c1] |
| 58 | 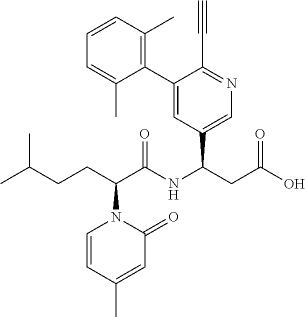 [SMILES: C#Cc1ncc([C@@H](CC(=O)O)NC(=O)[C@H](CCC(C)C)n2ccc(C)cc2=O)cc1-c1c(C)cccc1C] | 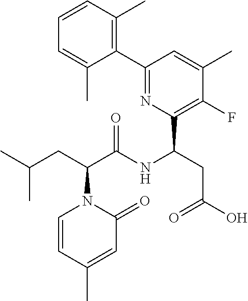 [SMILES: Cc1ccn([C@@H](CC(C)C)C(=O)N[C@H](CC(=O)O)c2nc(-c3c(C)cccc3C)cc(C)c2F)c(=O)c1] | 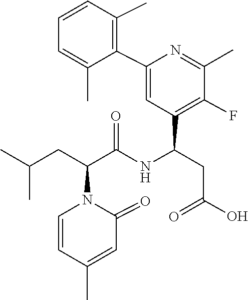 [SMILES: Cc1ccn([C@@H](CC(C)C)C(=O)N[C@H](CC(=O)O)c2cc(-c3c(C)cccc3C)nc(C)c2F)c(=O)c1] |
| 59 | 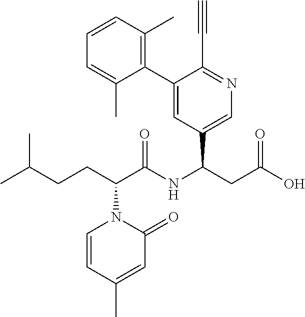 [SMILES: C#Cc1ncc([C@@H](CC(=O)O)NC(=O)[C@@H](CCC(C)C)n2ccc(C)cc2=O)cc1-c1c(C)cccc1C] | 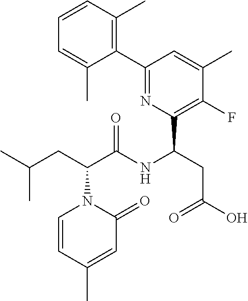 [SMILES: Cc1ccn([C@H](CC(C)C)C(=O)N[C@H](CC(=O)O)c2nc(-c3c(C)cccc3C)cc(C)c2F)c(=O)c1] | 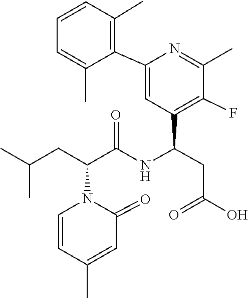 [SMILES: Cc1ccn([C@H](CC(C)C)C(=O)N[C@H](CC(=O)O)c2cc(-c3c(C)cccc3C)nc(C)c2F)c(=O)c1] |
| 60 | 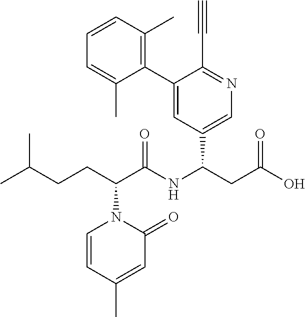 [SMILES: C#Cc1ncc([C@H](CC(=O)O)NC(=O)[C@@H](CCC(C)C)n2ccc(C)cc2=O)cc1-c1c(C)cccc1C] | 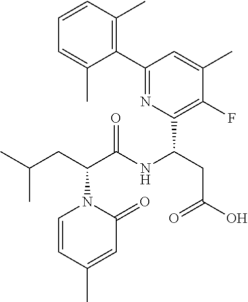 [SMILES: Cc1ccn([C@H](CC(C)C)C(=O)N[C@@H](CC(=O)O)c2nc(-c3c(C)cccc3C)cc(C)c2F)c(=O)c1] | 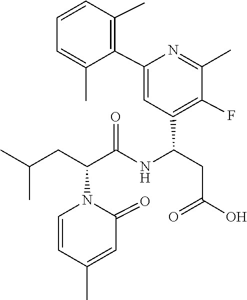 [SMILES: Cc1ccn([C@H](CC(C)C)C(=O)N[C@@H](CC(=O)O)c2cc(-c3c(C)cccc3C)nc(C)c2F)c(=O)c1] |
| 61 | 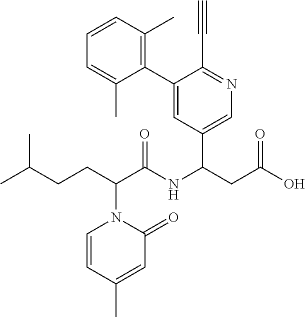 [SMILES: C#Cc1ncc(C(CC(=O)O)NC(=O)C(CCC(C)C)n2ccc(C)cc2=O)cc1-c1c(C)cccc1C] | 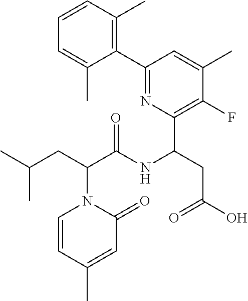 [SMILES: Cc1ccn(C(CC(C)C)C(=O)NC(CC(=O)O)c2nc(-c3c(C)cccc3C)cc(C)c2F)c(=O)c1] | 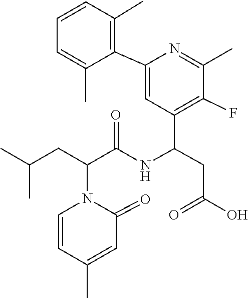 [SMILES: Cc1ccn(C(CC(C)C)C(=O)NC(CC(=O)O)c2cc(-c3c(C)cccc3C)nc(C)c2F)c(=O)c1] |
| 62 | 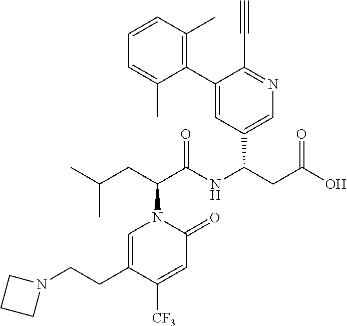 [SMILES: C#Cc1ncc([C@H](CC(=O)O)NC(=O)[C@H](CC(C)C)n2cc(CCN3CCC3)c(C(F)(F)F)cc2=O)cc1-c1c(C)cccc1C] | 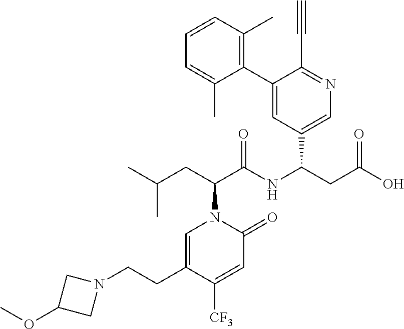 [SMILES: C#Cc1ncc([C@H](CC(=O)O)NC(=O)[C@H](CC(C)C)n2cc(CCN3CC(OC)C3)c(C(F)(F)F)cc2=O)cc1-c1c(C)cccc1C] | 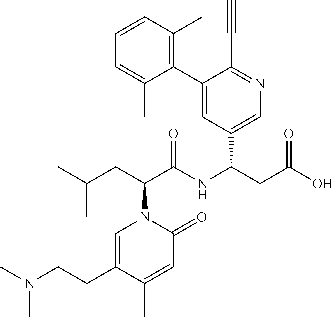 [SMILES: C#Cc1ncc([C@H](CC(=O)O)NC(=O)[C@H](CC(C)C)n2cc(CCN(C)C)c(C)cc2=O)cc1-c1c(C)cccc1C] |
| 63 | 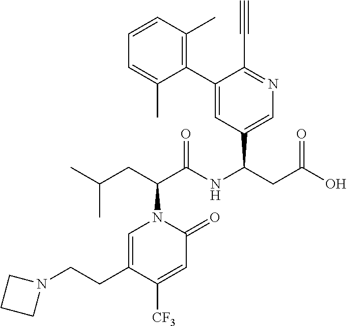 [SMILES: C#Cc1ncc([C@@H](CC(=O)O)NC(=O)[C@H](CC(C)C)n2cc(CCN3CCC3)c(C(F)(F)F)cc2=O)cc1-c1c(C)cccc1C] | 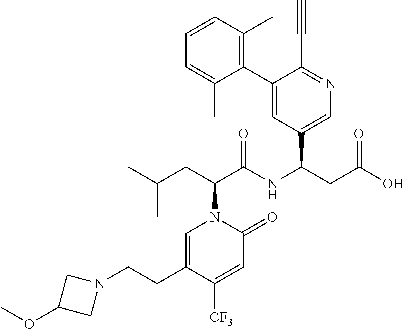 [SMILES: C#Cc1ncc([C@@H](CC(=O)O)NC(=O)[C@H](CC(C)C)n2cc(CCN3CC(OC)C3)c(C(F)(F)F)cc2=O)cc1-c1c(C)cccc1C] | 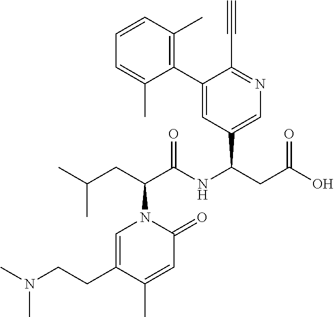 [SMILES: C#Cc1ncc([C@@H](CC(=O)O)NC(=O)[C@H](CC(C)C)n2cc(CCN(C)C)c(C)cc2=O)cc1-c1c(C)cccc1C] |
| 64 | 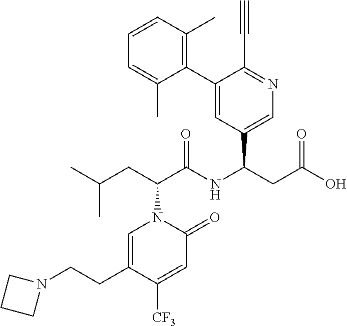 [SMILES: C#Cc1ncc([C@@H](CC(=O)O)NC(=O)[C@@H](CC(C)C)n2cc(CCN3CCC3)c(C(F)(F)F)cc2=O)cc1-c1c(C)cccc1C] | 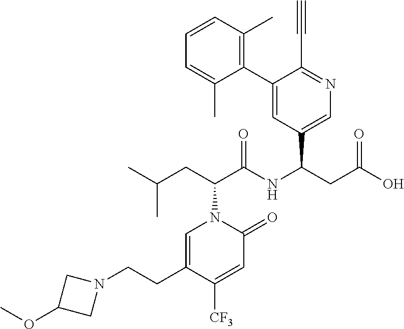 [SMILES: C#Cc1ncc([C@@H](CC(=O)O)NC(=O)[C@@H](CC(C)C)n2cc(CCN3CC(OC)C3)c(C(F)(F)F)cc2=O)cc1-c1c(C)cccc1C] | 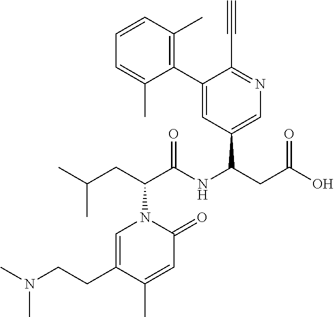 |
| 65 | 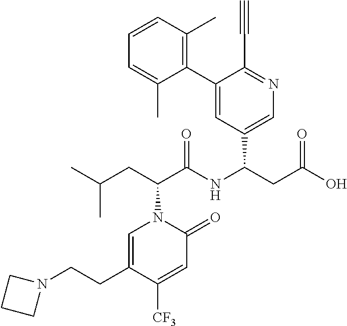 [SMILES: C#Cc1ncc([C@H](CC(=O)O)NC(=O)[C@@H](CC(C)C)n2cc(CCN3CCC3)c(C(F)(F)F)cc2=O)cc1-c1c(C)cccc1C] | 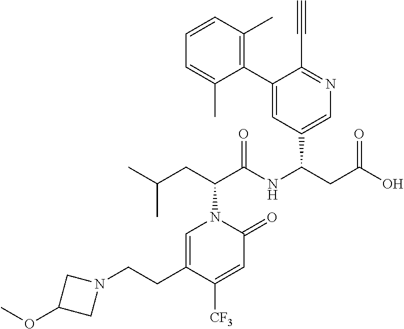 [SMILES: C#Cc1ncc([C@H](CC(=O)O)NC(=O)[C@@H](CC(C)C)n2cc(CCN3CC(OC)C3)c(C(F)(F)F)cc2=O)cc1-c1c(C)cccc1C] | 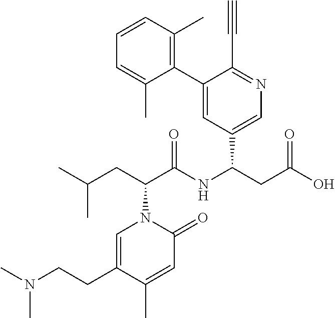 [SMILES: C#Cc1ncc([C@H](CC(=O)O)NC(=O)[C@@H](CC(C)C)n2cc(CCN(C)C)c(C)cc2=O)cc1-c1c(C)cccc1C] |
| 66 | 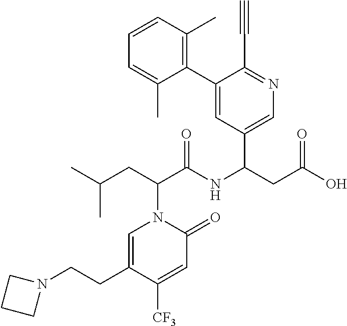 [SMILES: C#Cc1ncc(C(CC(=O)O)NC(=O)C(CC(C)C)n2cc(CCN3CCC3)c(C(F)(F)F)cc2=O)cc1-c1c(C)cccc1C] | 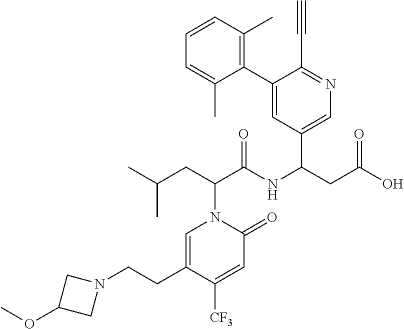 [SMILES: C#Cc1ncc(C(CC(=O)O)NC(=O)C(CC(C)C)n2cc(CCN3CC(OC)C3)c(C(F)(F)F)cc2=O)cc1-c1c(C)cccc1C] | 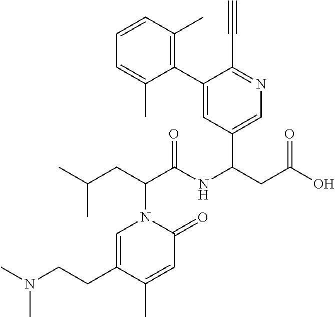 [SMILES: C#Cc1ncc(C(CC(=O)O)NC(=O)C(CC(C)C)n2cc(CCN(C)C)c(C)cc2=O)cc1-c1c(C)cccc1C] |
| 67 | 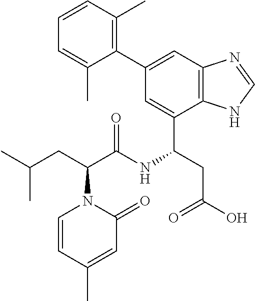 [SMILES: Cc1ccn([C@@H](CC(C)C)C(=O)N[C@@H](CC(=O)O)c2cc(-c3c(C)cccc3C)cc3nc[nH]c23)c(=O)c1] | 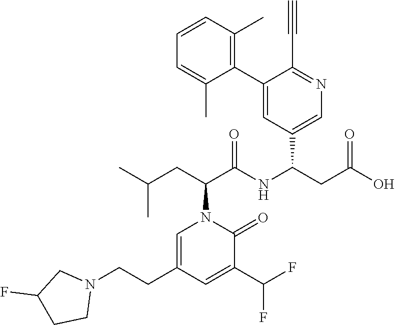 [SMILES: C#Cc1ncc([C@H](CC(=O)O)NC(=O)[C@H](CC(C)C)n2cc(CCN3CCC(F)C3)cc(C(F)F)c2=O)cc1-c1c(C)cccc1C] | 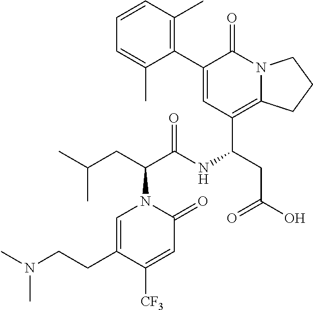 [SMILES: Cc1cccc(C)c1-c1cc([C@H](CC(=O)O)NC(=O)[C@H](CC(C)C)n2cc(CCN(C)C)c(C(F)(F)F)cc2=O)c2n(c1=O)CCC2] |
| 68 | 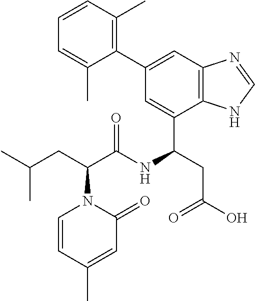 [SMILES: Cc1ccn([C@@H](CC(C)C)C(=O)N[C@H](CC(=O)O)c2cc(-c3c(C)cccc3C)cc3nc[nH]c23)c(=O)c1] | 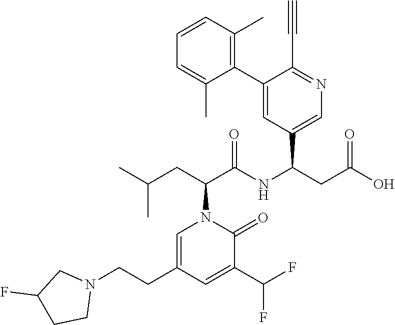 [SMILES: C#Cc1ncc([C@@H](CC(=O)O)NC(=O)[C@H](CC(C)C)n2cc(CCN3CCC(F)C3)cc(C(F)F)c2=O)cc1-c1c(C)cccc1C] | 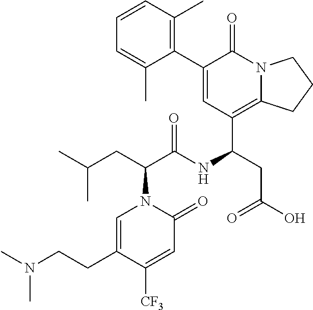 [SMILES: Cc1cccc(C)c1-c1cc([C@@H](CC(=O)O)NC(=O)[C@H](CC(C)C)n2cc(CCN(C)C)c(C(F)(F)F)cc2=O)c2n(c1=O)CCC2] |
| 69 | 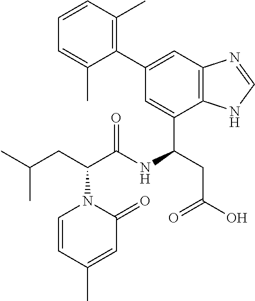 [SMILES: Cc1ccn([C@H](CC(C)C)C(=O)N[C@H](CC(=O)O)c2cc(-c3c(C)cccc3C)cc3nc[nH]c23)c(=O)c1] | 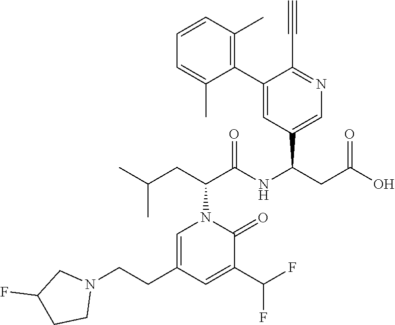 [SMILES: C#Cc1ncc([C@@H](CC(=O)O)NC(=O)[C@@H](CC(C)C)n2cc(CCN3CCC(F)C3)cc(C(F)F)c2=O)cc1-c1c(C)cccc1C] | 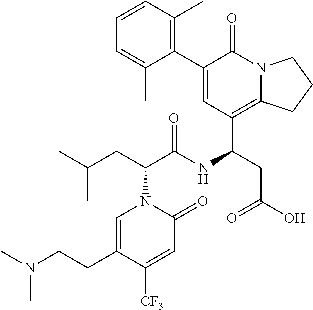 [SMILES: Cc1cccc(C)c1-c1cc([C@@H](CC(=O)O)NC(=O)[C@@H](CC(C)C)n2cc(CCN(C)C)c(C(F)(F)F)cc2=O)c2n(c1=O)CCC2] |
| 70 | 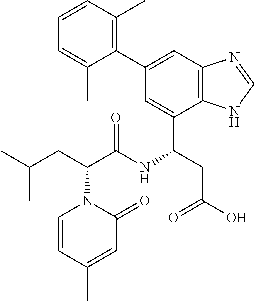 [SMILES: Cc1ccn([C@H](CC(C)C)C(=O)N[C@@H](CC(=O)O)c2cc(-c3c(C)cccc3C)cc3nc[nH]c23)c(=O)c1] | 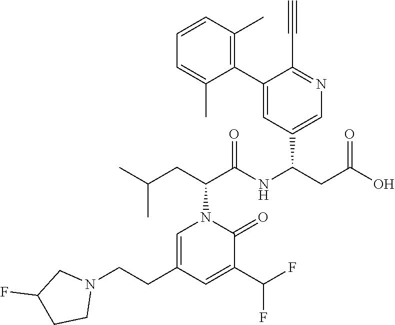 [SMILES: C#Cc1ncc([C@H](CC(=O)O)NC(=O)[C@@H](CC(C)C)n2cc(CCN3CCC(F)C3)cc(C(F)F)c2=O)cc1-c1c(C)cccc1C] | 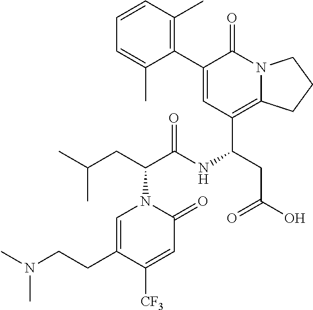 [SMILES: Cc1cccc(C)c1-c1cc([C@H](CC(=O)O)NC(=O)[C@@H](CC(C)C)n2cc(CCN(C)C)c(C(F)(F)F)cc2=O)c2n(c1=O)CCC2] |
| 71 | 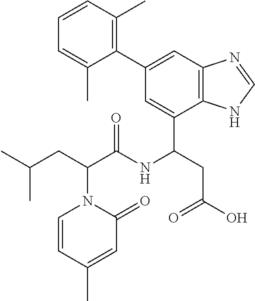 [SMILES: Cc1ccn(C(CC(C)C)C(=O)NC(CC(=O)O)c2cc(-c3c(C)cccc3C)cc3nc[nH]c23)c(=O)c1] | 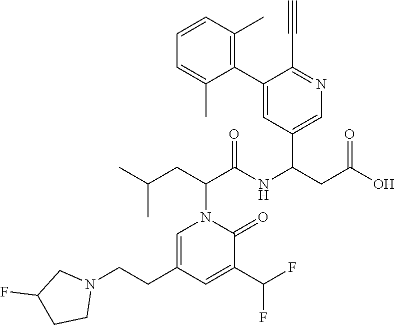 [SMILES: C#Cc1ncc(C(CC(=O)O)NC(=O)C(CC(C)C)n2cc(CCN3CCC(F)C3)cc(C(F)F)c2=O)cc1-c1c(C)cccc1C] | 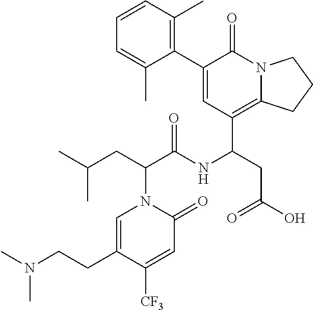 [SMILES: Cc1cccc(C)c1-c1cc(C(CC(=O)O)NC(=O)C(CC(C)C)n2cc(CCN(C)C)c(C(F)(F)F)cc2=O)c2n(c1=O)CCC2] |
| 72 | 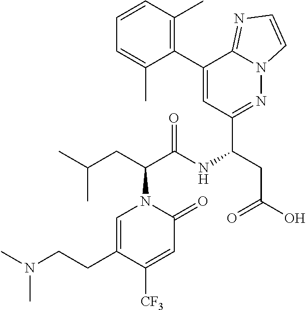 [SMILES: Cc1cccc(C)c1-c1cc([C@H](CC(=O)O)NC(=O)[C@H](CC(C)C)n2cc(CCN(C)C)c(C(F)(F)F)cc2=O)nn2ccnc12] | 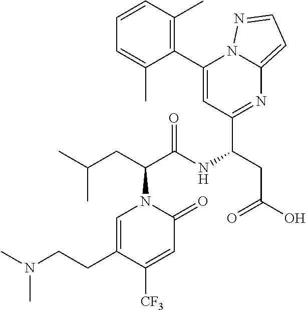 [SMILES: Cc1cccc(C)c1-c1cc([C@H](CC(=O)O)NC(=O)[C@H](CC(C)C)n2cc(CCN(C)C)c(C(F)(F)F)cc2=O)nc2ccnn12] | 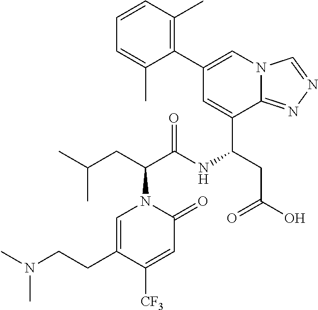 [SMILES: Cc1cccc(C)c1-c1cc([C@H](CC(=O)O)NC(=O)[C@H](CC(C)C)n2cc(CCN(C)C)c(C(F)(F)F)cc2=O)c2nncn2c1] |
| 73 | 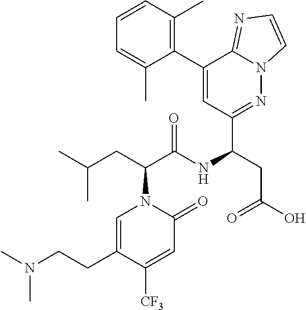 [SMILES: Cc1cccc(C)c1-c1cc([C@@H](CC(=O)O)NC(=O)[C@H](CC(C)C)n2cc(CCN(C)C)c(C(F)(F)F)cc2=O)nn2ccnc12] | 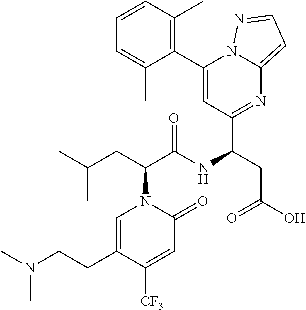 [SMILES: Cc1cccc(C)c1-c1cc([C@@H](CC(=O)O)NC(=O)[C@H](CC(C)C)n2cc(CCN(C)C)c(C(F)(F)F)cc2=O)nc2ccnn12] | 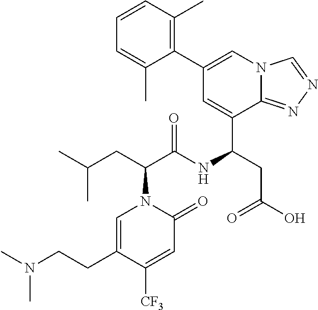 [SMILES: Cc1cccc(C)c1-c1cc([C@@H](CC(=O)O)NC(=O)[C@H](CC(C)C)n2cc(CCN(C)C)c(C(F)(F)F)cc2=O)c2nncn2c1] |
| 74 | 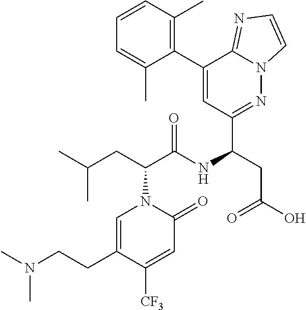 [SMILES: Cc1cccc(C)c1-c1cc([C@@H](CC(=O)O)NC(=O)[C@@H](CC(C)C)n2cc(CCN(C)C)c(C(F)(F)F)cc2=O)nn2ccnc12] | 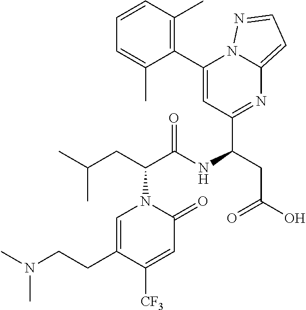 [SMILES: Cc1cccc(C)c1-c1cc([C@@H](CC(=O)O)NC(=O)[C@@H](CC(C)C)n2cc(CCN(C)C)c(C(F)(F)F)cc2=O)nc2ccnn12] | 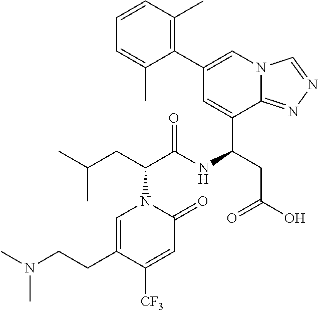 [SMILES: Cc1cccc(C)c1-c1cc([C@@H](CC(=O)O)NC(=O)[C@@H](CC(C)C)n2cc(CCN(C)C)c(C(F)(F)F)cc2=O)c2nncn2c1] |
| 75 | 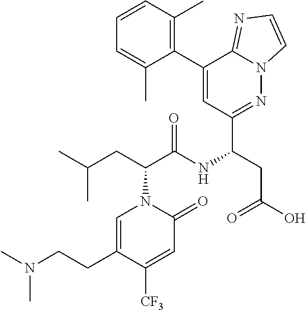 [SMILES: Cc1cccc(C)c1-c1cc([C@H](CC(=O)O)NC(=O)[C@@H](CC(C)C)n2cc(CCN(C)C)c(C(F)(F)F)cc2=O)nn2ccnc12] | 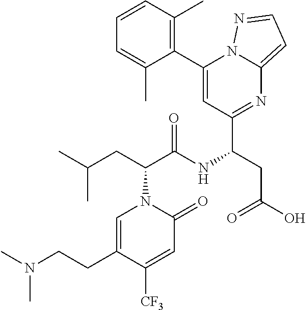 [SMILES: Cc1cccc(C)c1-c1cc([C@H](CC(=O)O)NC(=O)[C@@H](CC(C)C)n2cc(CCN(C)C)c(C(F)(F)F)cc2=O)nc2ccnn12] | 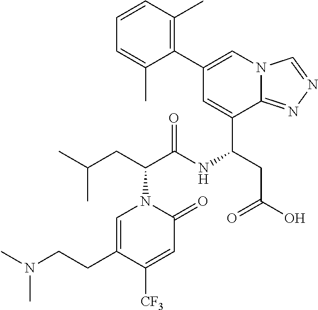 [SMILES: Cc1cccc(C)c1-c1cc([C@H](CC(=O)O)NC(=O)[C@@H](CC(C)C)n2cc(CCN(C)C)c(C(F)(F)F)cc2=O)c2nncn2c1] |
| 76 | 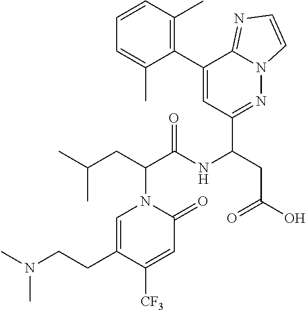 [SMILES: Cc1cccc(C)c1-c1cc(C(CC(=O)O)NC(=O)C(CC(C)C)n2cc(CCN(C)C)c(C(F)(F)F)cc2=O)nn2ccnc12] | 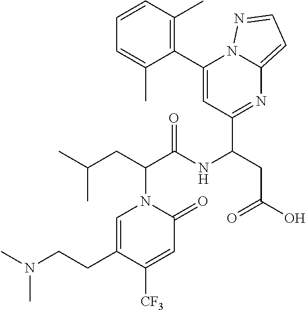 [SMILES: Cc1cccc(C)c1-c1cc(C(CC(=O)O)NC(=O)C(CC(C)C)n2cc(CCN(C)C)c(C(F)(F)F)cc2=O)nc2ccnn12] | 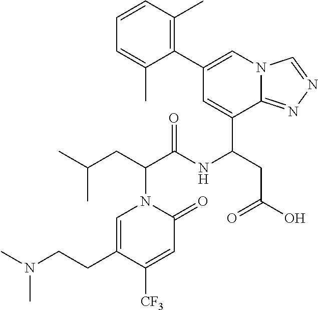 [SMILES: Cc1cccc(C)c1-c1cc(C(CC(=O)O)NC(=O)C(CC(C)C)n2cc(CCN(C)C)c(C(F)(F)F)cc2=O)c2nncn2c1] |
| 77 | 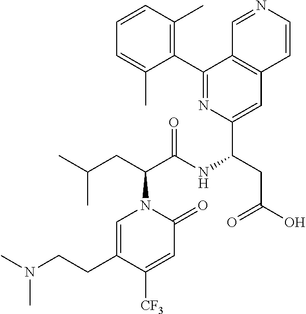 [SMILES: Cc1cccc(C)c1-c1nc([C@H](CC(=O)O)NC(=O)[C@H](CC(C)C)n2cc(CCN(C)C)c(C(F)(F)F)cc2=O)cc2ccncc12] | 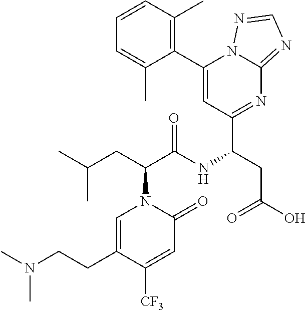 [SMILES: Cc1cccc(C)c1-c1cc([C@H](CC(=O)O)NC(=O)[C@H](CC(C)C)n2cc(CCN(C)C)c(C(F)(F)F)cc2=O)nc2ncnn12] | 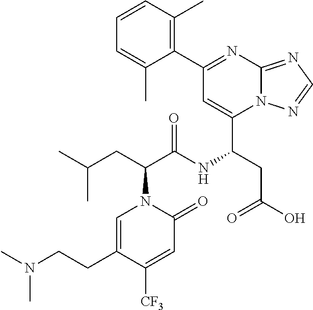 [SMILES: Cc1cccc(C)c1-c1cc([C@H](CC(=O)O)NC(=O)[C@H](CC(C)C)n2cc(CCN(C)C)c(C(F)(F)F)cc2=O)n2ncnc2n1] |
| 78 | 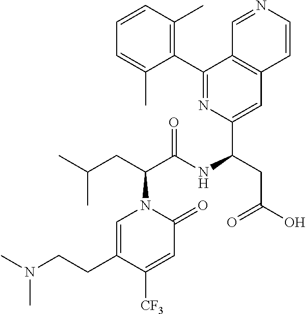 [SMILES: Cc1cccc(C)c1-c1nc([C@@H](CC(=O)O)NC(=O)[C@H](CC(C)C)n2cc(CCN(C)C)c(C(F)(F)F)cc2=O)cc2ccncc12] | 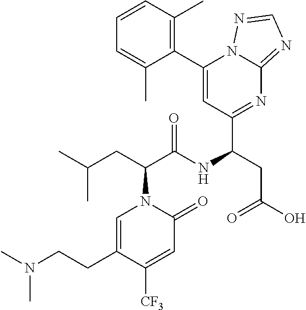 [SMILES: Cc1cccc(C)c1-c1cc([C@@H](CC(=O)O)NC(=O)[C@H](CC(C)C)n2cc(CCN(C)C)c(C(F)(F)F)cc2=O)nc2ncnn12] | 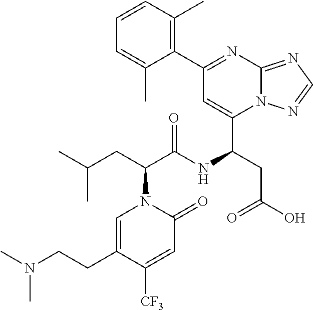 [SMILES: Cc1cccc(C)c1-c1cc([C@@H](CC(=O)O)NC(=O)[C@H](CC(C)C)n2cc(CCN(C)C)c(C(F)(F)F)cc2=O)n2ncnc2n1] |
| 79 | 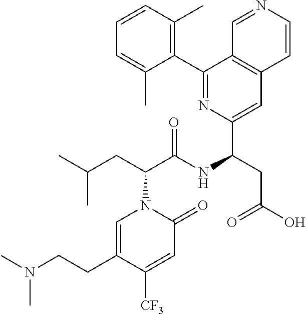 [SMILES: Cc1cccc(C)c1-c1nc([C@@H](CC(=O)O)NC(=O)[C@@H](CC(C)C)n2cc(CCN(C)C)c(C(F)(F)F)cc2=O)cc2ccncc12] | 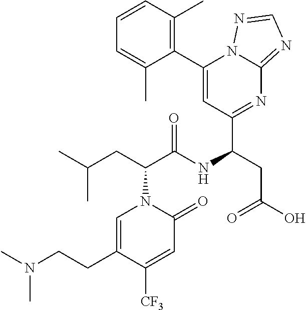 [SMILES: Cc1cccc(C)c1-c1cc([C@@H](CC(=O)O)NC(=O)[C@@H](CC(C)C)n2cc(CCN(C)C)c(C(F)(F)F)cc2=O)nc2ncnn12] | 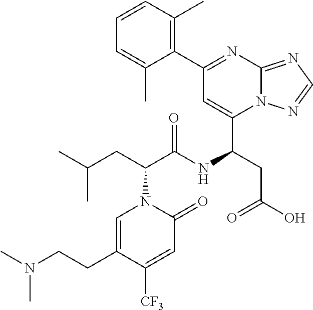 [SMILES: Cc1cccc(C)c1-c1cc([C@@H](CC(=O)O)NC(=O)[C@@H](CC(C)C)n2cc(CCN(C)C)c(C(F)(F)F)cc2=O)n2ncnc2n1] |
| 80 | 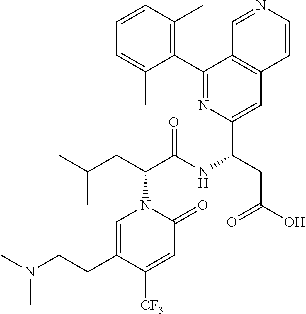 [SMILES: Cc1cccc(C)c1-c1nc([C@H](CC(=O)O)NC(=O)[C@@H](CC(C)C)n2cc(CCN(C)C)c(C(F)(F)F)cc2=O)cc2ccncc12] | 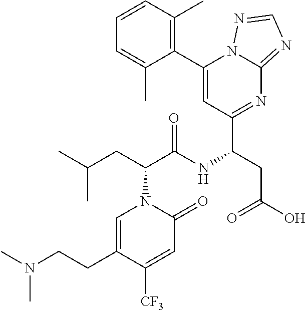 [SMILES: Cc1cccc(C)c1-c1cc([C@H](CC(=O)O)NC(=O)[C@@H](CC(C)C)n2cc(CCN(C)C)c(C(F)(F)F)cc2=O)nc2ncnn12] | 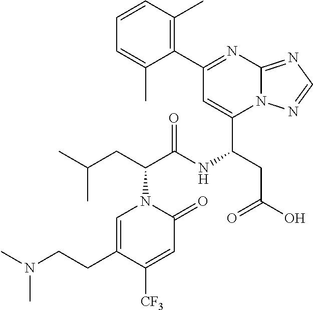 [SMILES: Cc1cccc(C)c1-c1cc([C@H](CC(=O)O)NC(=O)[C@@H](CC(C)C)n2cc(CCN(C)C)c(C(F)(F)F)cc2=O)n2ncnc2n1] |
| 81 | 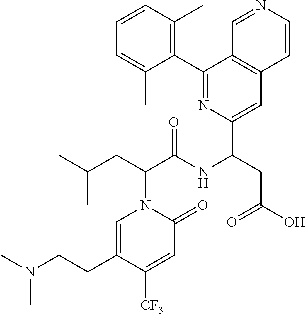 [SMILES: Cc1cccc(C)c1-c1nc(C(CC(=O)O)NC(=O)C(CC(C)C)n2cc(CCN(C)C)c(C(F)(F)F)cc2=O)cc2ccncc12] | 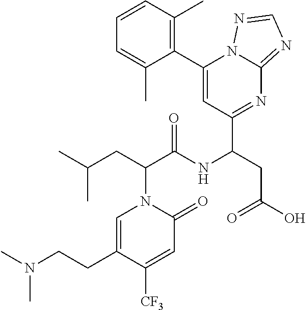 [SMILES: Cc1cccc(C)c1-c1cc(C(CC(=O)O)NC(=O)C(CC(C)C)n2cc(CCN(C)C)c(C(F)(F)F)cc2=O)nc2ncnn12] | 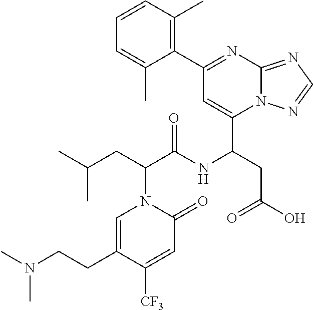 [SMILES: Cc1cccc(C)c1-c1cc(C(CC(=O)O)NC(=O)C(CC(C)C)n2cc(CCN(C)C)c(C(F)(F)F)cc2=O)n2ncnc2n1] |
| 82 | 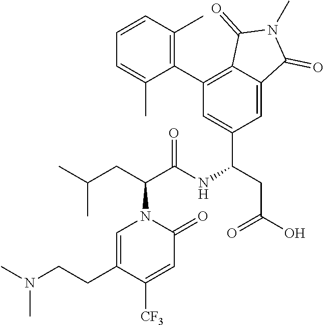 [SMILES: Cc1cccc(C)c1-c1cc([C@H](CC(=O)O)NC(=O)[C@H](CC(C)C)n2cc(CCN(C)C)c(C(F)(F)F)cc2=O)cc2c1C(=O)N(C)C2=O] | 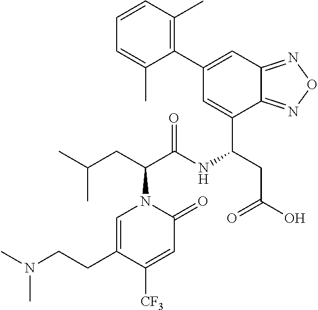 [SMILES: Cc1cccc(C)c1-c1cc([C@H](CC(=O)O)NC(=O)[C@H](CC(C)C)n2cc(CCN(C)C)c(C(F)(F)F)cc2=O)c2nonc2c1] | 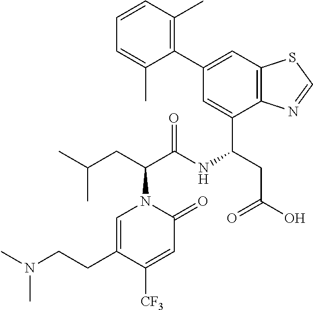 [SMILES: Cc1cccc(C)c1-c1cc([C@H](CC(=O)O)NC(=O)[C@H](CC(C)C)n2cc(CCN(C)C)c(C(F)(F)F)cc2=O)c2ncsc2c1] |
| 83 | 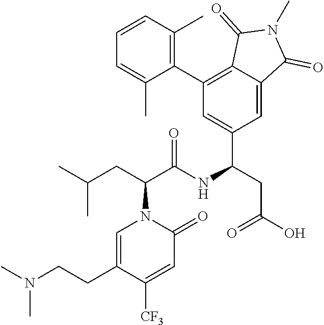 [SMILES: Cc1cccc(C)c1-c1cc([C@@H](CC(=O)O)NC(=O)[C@H](CC(C)C)n2cc(CCN(C)C)c(C(F)(F)F)cc2=O)cc2c1C(=O)N(C)C2=O] | 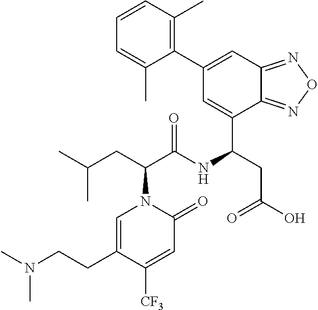 [SMILES: Cc1cccc(C)c1-c1cc([C@@H](CC(=O)O)NC(=O)[C@H](CC(C)C)n2cc(CCN(C)C)c(C(F)(F)F)cc2=O)c2nonc2c1] | 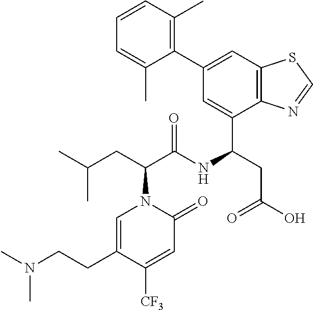 [SMILES: Cc1cccc(C)c1-c1cc([C@@H](CC(=O)O)NC(=O)[C@H](CC(C)C)n2cc(CCN(C)C)c(C(F)(F)F)cc2=O)c2ncsc2c1] |
| 84 | 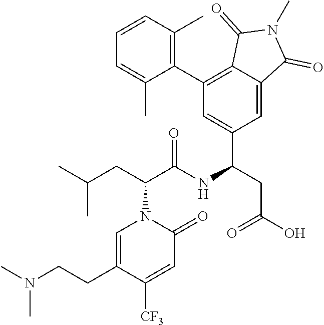 [SMILES: Cc1cccc(C)c1-c1cc([C@@H](CC(=O)O)NC(=O)[C@@H](CC(C)C)n2cc(CCN(C)C)c(C(F)(F)F)cc2=O)cc2c1C(=O)N(C)C2=O] | 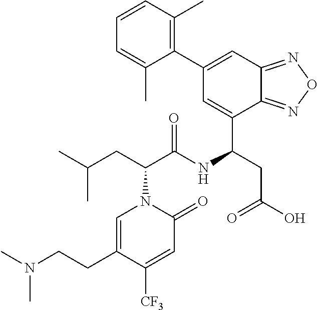 [SMILES: Cc1cccc(C)c1-c1cc([C@@H](CC(=O)O)NC(=O)[C@@H](CC(C)C)n2cc(CCN(C)C)c(C(F)(F)F)cc2=O)c2nonc2c1] | 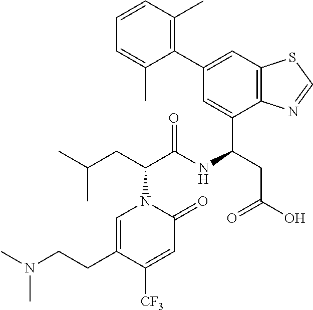 [SMILES: Cc1cccc(C)c1-c1cc([C@@H](CC(=O)O)NC(=O)[C@@H](CC(C)C)n2cc(CCN(C)C)c(C(F)(F)F)cc2=O)c2ncsc2c1] |
| 85 | 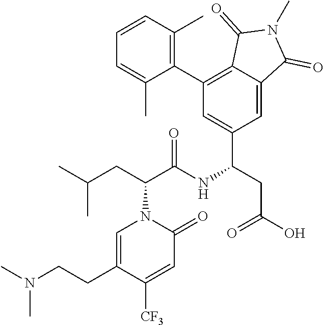 [SMILES: Cc1cccc(C)c1-c1cc([C@H](CC(=O)O)NC(=O)[C@@H](CC(C)C)n2cc(CCN(C)C)c(C(F)(F)F)cc2=O)cc2c1C(=O)N(C)C2=O] | 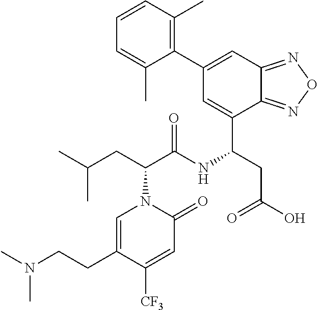 [SMILES: Cc1cccc(C)c1-c1cc([C@H](CC(=O)O)NC(=O)[C@@H](CC(C)C)n2cc(CCN(C)C)c(C(F)(F)F)cc2=O)c2nonc2c1] | 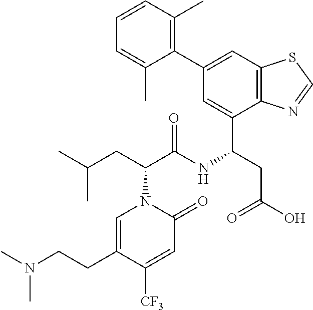 [SMILES: Cc1cccc(C)c1-c1cc([C@H](CC(=O)O)NC(=O)[C@@H](CC(C)C)n2cc(CCN(C)C)c(C(F)(F)F)cc2=O)c2ncsc2c1] |
| 86 | 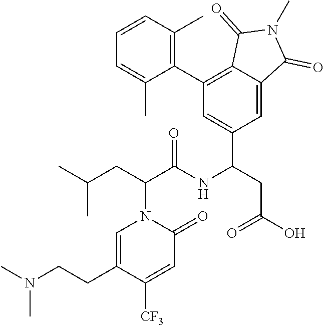 [SMILES: Cc1cccc(C)c1-c1cc(C(CC(=O)O)NC(=O)C(CC(C)C)n2cc(CCN(C)C)c(C(F)(F)F)cc2=O)cc2c1C(=O)N(C)C2=O] | 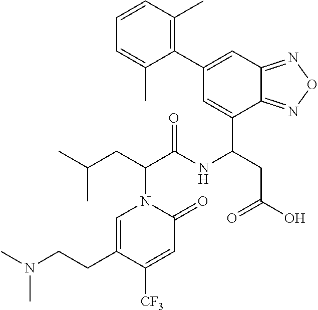 [SMILES: Cc1cccc(C)c1-c1cc(C(CC(=O)O)NC(=O)C(CC(C)C)n2cc(CCN(C)C)c(C(F)(F)F)cc2=O)c2nonc2c1] | 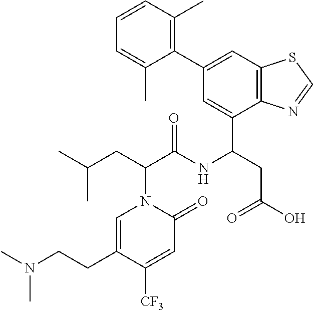 [SMILES: Cc1cccc(C)c1-c1cc(C(CC(=O)O)NC(=O)C(CC(C)C)n2cc(CCN(C)C)c(C(F)(F)F)cc2=O)c2ncsc2c1] |
| 87 | 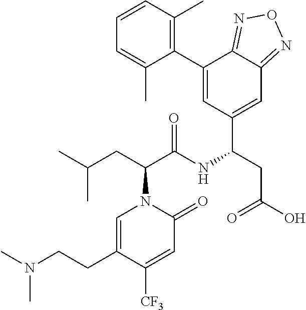 [SMILES: Cc1cccc(C)c1-c1cc([C@H](CC(=O)O)NC(=O)[C@H](CC(C)C)n2cc(CCN(C)C)c(C(F)(F)F)cc2=O)cc2nonc12] | 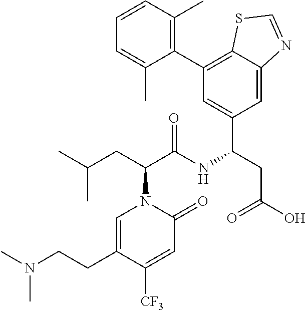 [SMILES: Cc1cccc(C)c1-c1cc([C@H](CC(=O)O)NC(=O)[C@H](CC(C)C)n2cc(CCN(C)C)c(C(F)(F)F)cc2=O)cc2ncsc12] | 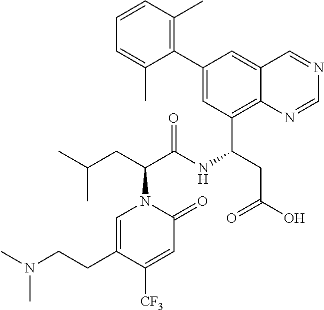 [SMILES: Cc1cccc(C)c1-c1cc([C@H](CC(=O)O)NC(=O)[C@H](CC(C)C)n2cc(CCN(C)C)c(C(F)(F)F)cc2=O)c2ncncc2c1] |
| 88 | 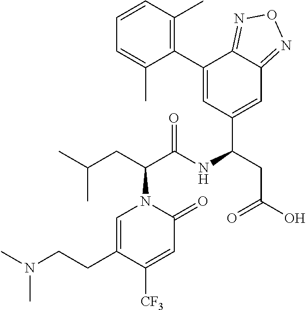 [SMILES: Cc1cccc(C)c1-c1cc([C@@H](CC(=O)O)NC(=O)[C@H](CC(C)C)n2cc(CCN(C)C)c(C(F)(F)F)cc2=O)cc2nonc12] | 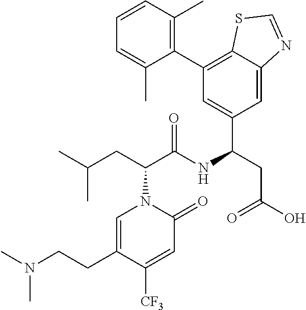 [SMILES: Cc1cccc(C)c1-c1cc([C@@H](CC(=O)O)NC(=O)[C@@H](CC(C)C)n2cc(CCN(C)C)c(C(F)(F)F)cc2=O)cc2ncsc12] | 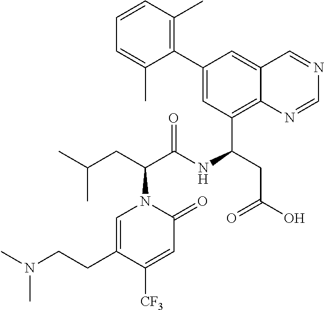 [SMILES: Cc1cccc(C)c1-c1cc([C@@H](CC(=O)O)NC(=O)[C@H](CC(C)C)n2cc(CCN(C)C)c(C(F)(F)F)cc2=O)c2ncncc2c1] |
| 89 | 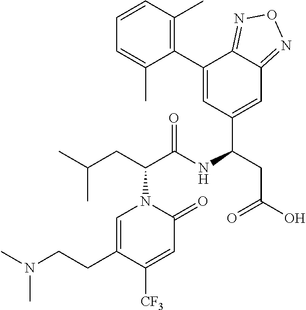 [SMILES: Cc1cccc(C)c1-c1cc([C@@H](CC(=O)O)NC(=O)[C@@H](CC(C)C)n2cc(CCN(C)C)c(C(F)(F)F)cc2=O)cc2nonc12] | 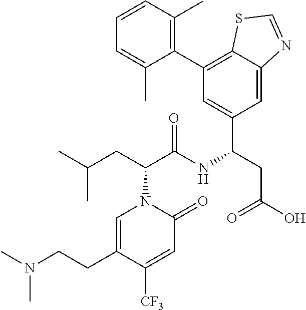 [SMILES: Cc1cccc(C)c1-c1cc([C@H](CC(=O)O)NC(=O)[C@@H](CC(C)C)n2cc(CCN(C)C)c(C(F)(F)F)cc2=O)cc2ncsc12] | 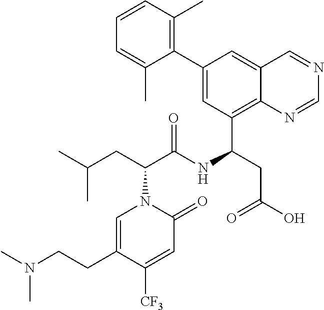 [SMILES: Cc1cccc(C)c1-c1cc([C@@H](CC(=O)O)NC(=O)[C@@H](CC(C)C)n2cc(CCN(C)C)c(C(F)(F)F)cc2=O)c2ncncc2c1] |
| 90 | 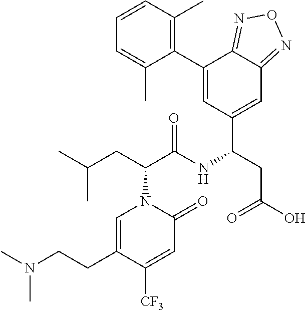 [SMILES: Cc1cccc(C)c1-c1cc([C@H](CC(=O)O)NC(=O)[C@@H](CC(C)C)n2cc(CCN(C)C)c(C(F)(F)F)cc2=O)cc2nonc12] | 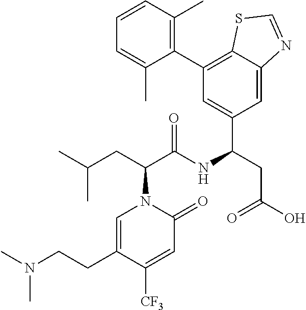 [SMILES: Cc1cccc(C)c1-c1cc([C@@H](CC(=O)O)NC(=O)[C@H](CC(C)C)n2cc(CCN(C)C)c(C(F)(F)F)cc2=O)cc2ncsc12] | 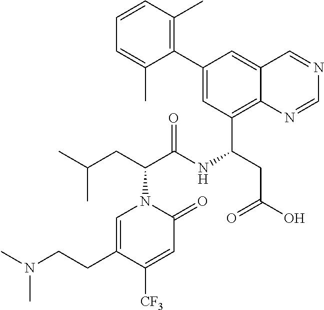 [SMILES: Cc1cccc(C)c1-c1cc([C@H](CC(=O)O)NC(=O)[C@@H](CC(C)C)n2cc(CCN(C)C)c(C(F)(F)F)cc2=O)c2ncncc2c1] |
| 91 | 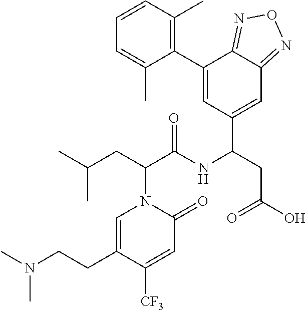 [SMILES: Cc1cccc(C)c1-c1cc(C(CC(=O)O)NC(=O)C(CC(C)C)n2cc(CCN(C)C)c(C(F)(F)F)cc2=O)cc2nonc12] | 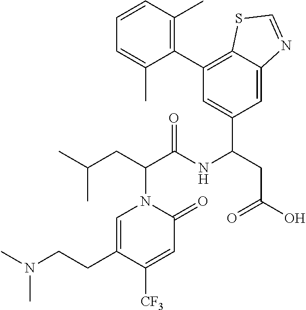 [SMILES: Cc1cccc(C)c1-c1cc(C(CC(=O)O)NC(=O)C(CC(C)C)n2cc(CCN(C)C)c(C(F)(F)F)cc2=O)cc2ncsc12] | 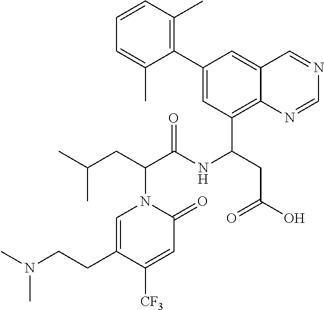 [SMILES: Cc1cccc(C)c1-c1cc(C(CC(=O)O)NC(=O)C(CC(C)C)n2cc(CCN(C)C)c(C(F)(F)F)cc2=O)c2ncncc2c1] |
| 92 | 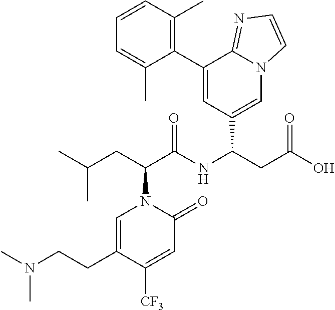 [SMILES: Cc1cccc(C)c1-c1cc([C@H](CC(=O)O)NC(=O)[C@H](CC(C)C)n2cc(CCN(C)C)c(C(F)(F)F)cc2=O)cn2ccnc12] | 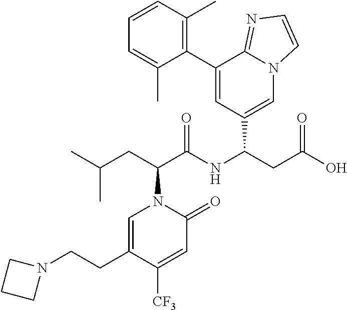 [SMILES: Cc1cccc(C)c1-c1cc([C@H](CC(=O)O)NC(=O)[C@H](CC(C)C)n2cc(CCN3CCC3)c(C(F)(F)F)cc2=O)cn2ccnc12] | 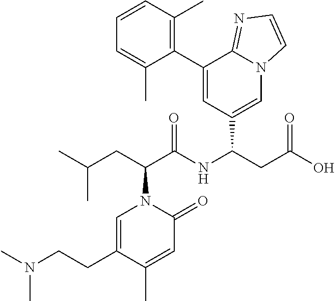 [SMILES: Cc1cc(=O)n([C@@H](CC(C)C)C(=O)N[C@@H](CC(=O)O)c2cc(-c3c(C)cccc3C)c3nccn3c2)cc1CCN(C)C] |
| 93 | 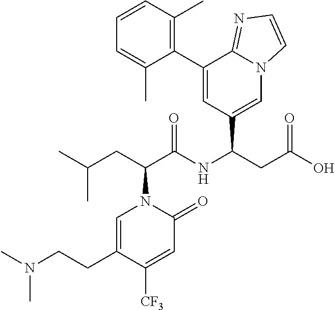 [SMILES: Cc1cccc(C)c1-c1cc([C@@H](CC(=O)O)NC(=O)[C@H](CC(C)C)n2cc(CCN(C)C)c(C(F)(F)F)cc2=O)cn2ccnc12] | 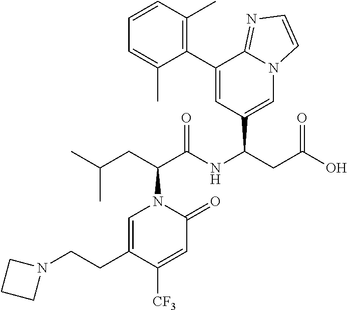 [SMILES: Cc1cccc(C)c1-c1cc([C@@H](CC(=O)O)NC(=O)[C@H](CC(C)C)n2cc(CCN3CCC3)c(C(F)(F)F)cc2=O)cn2ccnc12] | 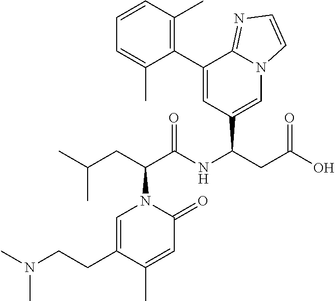 [SMILES: Cc1cc(=O)n([C@@H](CC(C)C)C(=O)N[C@H](CC(=O)O)c2cc(-c3c(C)cccc3C)c3nccn3c2)cc1CCN(C)C] |
| 94 | 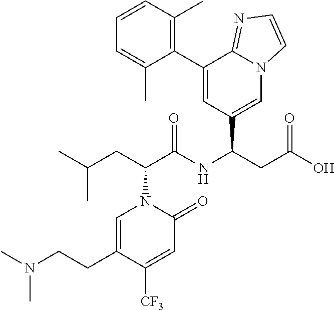 [SMILES: Cc1cccc(C)c1-c1cc([C@@H](CC(=O)O)NC(=O)[C@@H](CC(C)C)n2cc(CCN(C)C)c(C(F)(F)F)cc2=O)cn2ccnc12] | 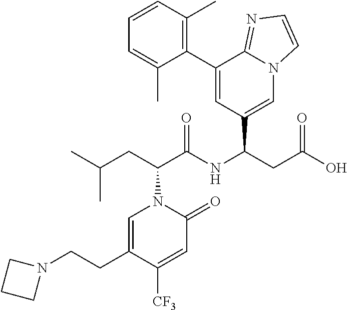 [SMILES: Cc1cccc(C)c1-c1cc([C@@H](CC(=O)O)NC(=O)[C@@H](CC(C)C)n2cc(CCN3CCC3)c(C(F)(F)F)cc2=O)cn2ccnc12] | 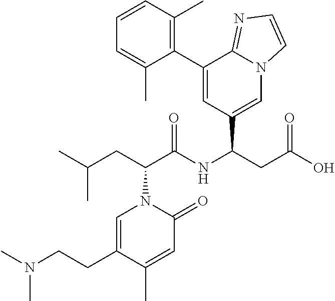 [SMILES: Cc1cc(=O)n([C@H](CC(C)C)C(=O)N[C@H](CC(=O)O)c2cc(-c3c(C)cccc3C)c3nccn3c2)cc1CCN(C)C] |
| 95 | 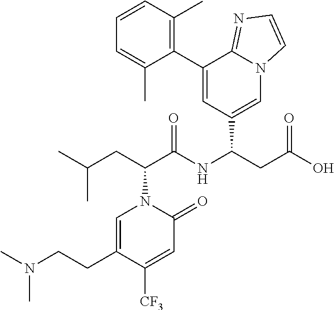 [SMILES: Cc1cccc(C)c1-c1cc([C@H](CC(=O)O)NC(=O)[C@@H](CC(C)C)n2cc(CCN(C)C)c(C(F)(F)F)cc2=O)cn2ccnc12] | 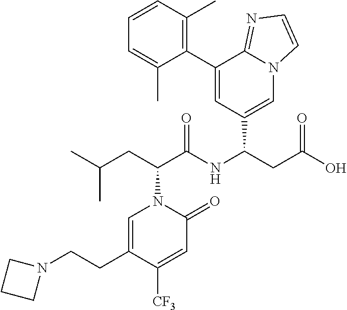 [SMILES: Cc1cccc(C)c1-c1cc([C@H](CC(=O)O)NC(=O)[C@@H](CC(C)C)n2cc(CCN3CCC3)c(C(F)(F)F)cc2=O)cn2ccnc12] | 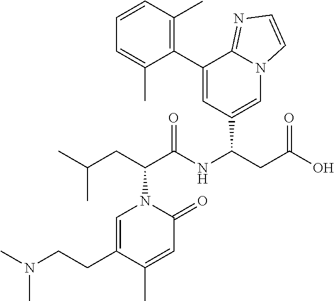 [SMILES: Cc1cc(=O)n([C@H](CC(C)C)C(=O)N[C@@H](CC(=O)O)c2cc(-c3c(C)cccc3C)c3nccn3c2)cc1CCN(C)C] |
| 96 | 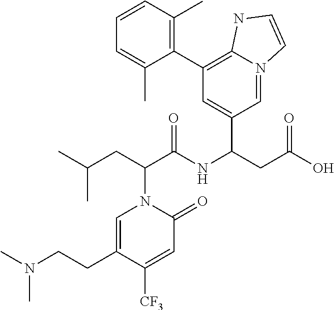 | 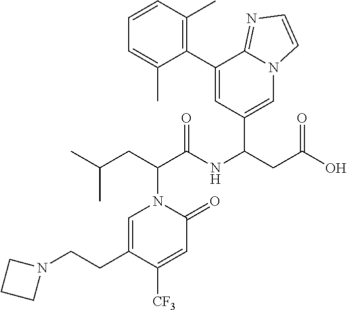 [SMILES: Cc1cccc(C)c1-c1cc(C(CC(=O)O)NC(=O)C(CC(C)C)n2cc(CCN3CCC3)c(C(F)(F)F)cc2=O)cn2ccnc12] | 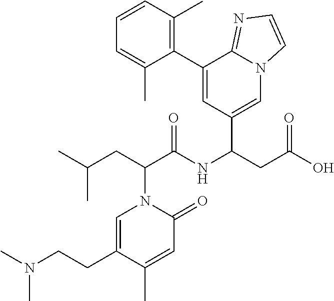 [SMILES: Cc1cc(=O)n(C(CC(C)C)C(=O)NC(CC(=O)O)c2cc(-c3c(C)cccc3C)c3nccn3c2)cc1CCN(C)C] |
| 97 | 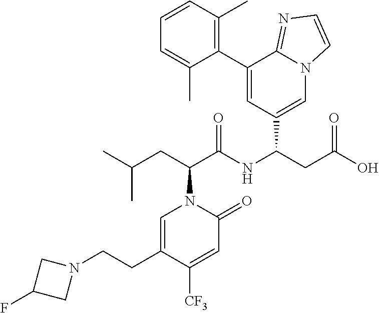 [SMILES: Cc1cccc(C)c1-c1cc([C@H](CC(=O)O)NC(=O)[C@H](CC(C)C)n2cc(CCN3CC(F)C3)c(C(F)(F)F)cc2=O)cn2ccnc12] | 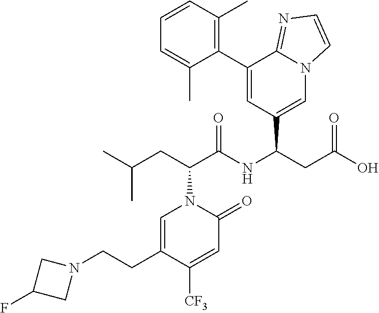 [SMILES: Cc1cccc(C)c1-c1cc([C@@H](CC(=O)O)NC(=O)[C@@H](CC(C)C)n2cc(CCN3CC(F)C3)c(C(F)(F)F)cc2=O)cn2ccnc12] | 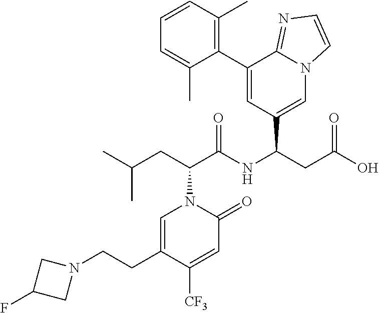 [SMILES: Cc1cccc(C)c1-c1cc([C@@H](CC(=O)O)NC(=O)[C@@H](CC(C)C)n2cc(CCN3CC(F)C3)c(C(F)(F)F)cc2=O)cn2ccnc12] |
| 98 | 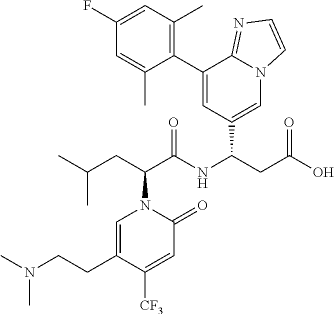 [SMILES: Cc1cc(F)cc(C)c1-c1cc([C@H](CC(=O)O)NC(=O)[C@H](CC(C)C)n2cc(CCN(C)C)c(C(F)(F)F)cc2=O)cn2ccnc12] | 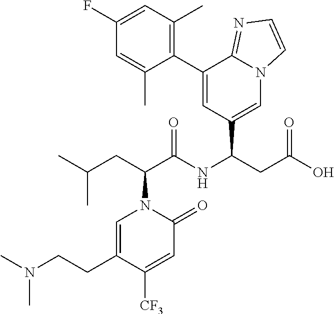 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)cn2ccnc12] | 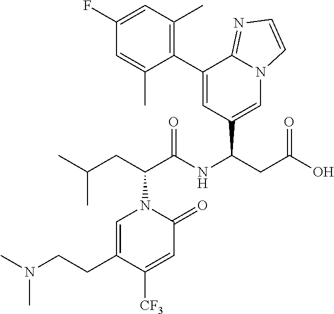 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)cn2ccnc12] |
| 99 | 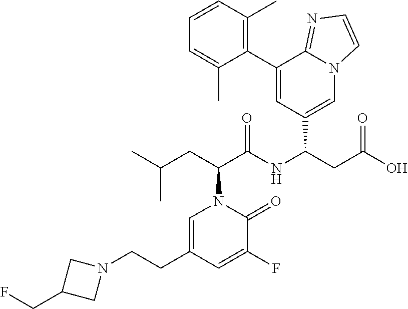 [SMILES: Cc1cccc(C)c1-c1cc([C@H](CC(=O)O)NC(=O)[C@H](CC(C)C)n2cc(CCN3CC(CF)C3)cc(F)c2=O)cn2ccnc12] | 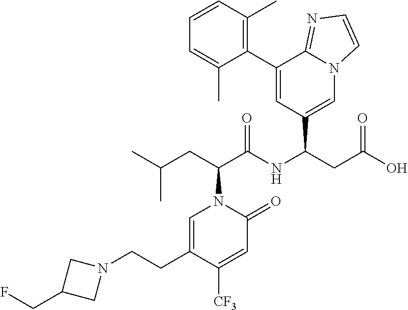 [SMILES: Cc1cccc(C)c1-c1cc([C@@H](CC(=O)O)NC(=O)[C@H](CC(C)C)n2cc(CCN3CC(CF)C3)c(C(F)(F)F)cc2=O)cn2ccnc12] | 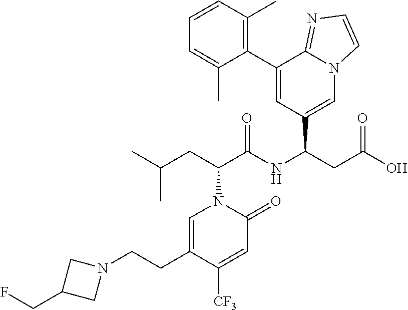 [SMILES: Cc1cccc(C)c1-c1cc([C@@H](CC(=O)O)NC(=O)[C@@H](CC(C)C)n2cc(CCN3CC(CF)C3)c(C(F)(F)F)cc2=O)cn2ccnc12] |
| 100 | 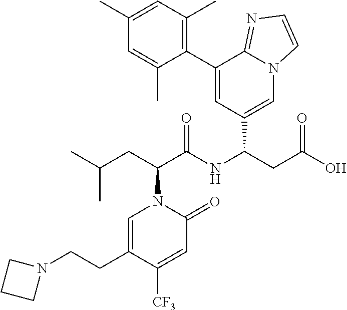 [SMILES: Cc1cc(C)c(-c2cc([C@H](CC(=O)O)NC(=O)[C@H](CC(C)C)n3cc(CCN4CCC4)c(C(F)(F)F)cc3=O)cn3ccnc23)c(C)c1] | 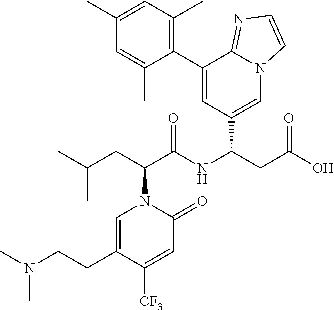 [SMILES: Cc1cc(C)c(-c2cc([C@H](CC(=O)O)NC(=O)[C@H](CC(C)C)n3cc(CCN(C)C)c(C(F)(F)F)cc3=O)cn3ccnc23)c(C)c1] | 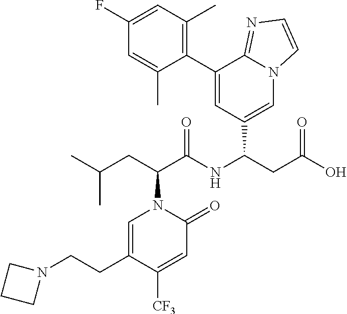 [SMILES: Cc1cc(F)cc(C)c1-c1cc([C@H](CC(=O)O)NC(=O)[C@H](CC(C)C)n2cc(CCN3CCC3)c(C(F)(F)F)cc2=O)cn2ccnc12] |
| 101 | 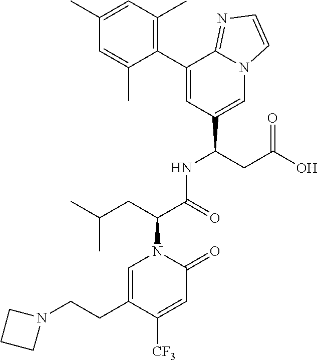 [SMILES: Cc1cc(C)c(-c2cc([C@@H](CC(=O)O)NC(=O)[C@H](CC(C)C)n3cc(CCN4CCC4)c(C(F)(F)F)cc3=O)cn3ccnc23)c(C)c1] | 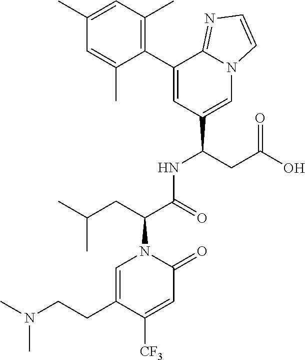 [SMILES: Cc1cc(C)c(-c2cc([C@@H](CC(=O)O)NC(=O)[C@H](CC(C)C)n3cc(CCN(C)C)c(C(F)(F)F)cc3=O)cn3ccnc23)c(C)c1] | 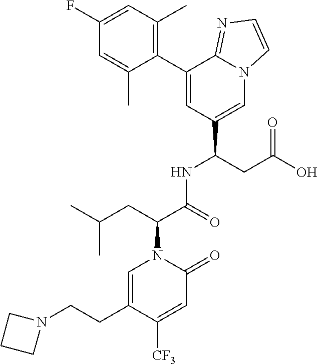 [SMILES: Cc1cc(F)cc(C)c1-c1cc([C@@H](CC(=O)O)NC(=O)[C@H](CC(C)C)n2cc(CCN3CCC3)c(C(F)(F)F)cc2=O)cn2ccnc12] |
| 102 | 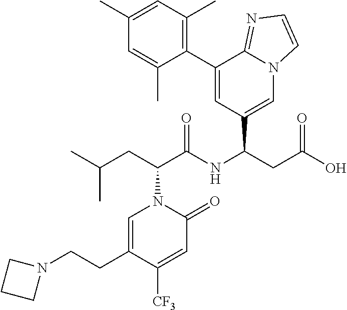 [SMILES: Cc1cc(C)c(-c2cc([C@@H](CC(=O)O)NC(=O)[C@@H](CC(C)C)n3cc(CCN4CCC4)c(C(F)(F)F)cc3=O)cn3ccnc23)c(C)c1] | 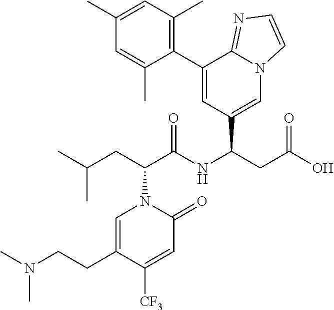 [SMILES: Cc1cc(C)c(-c2cc([C@@H](CC(=O)O)NC(=O)[C@@H](CC(C)C)n3cc(CCN(C)C)c(C(F)(F)F)cc3=O)cn3ccnc23)c(C)c1] | 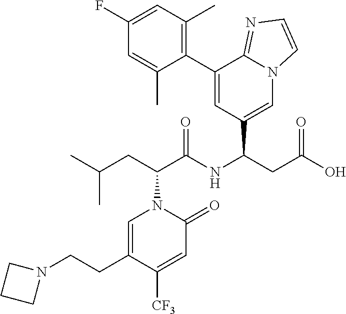 [SMILES: Cc1cc(F)cc(C)c1-c1cc([C@@H](CC(=O)O)NC(=O)[C@@H](CC(C)C)n2cc(CCN3CCC3)c(C(F)(F)F)cc2=O)cn2ccnc12] |
| 103 | 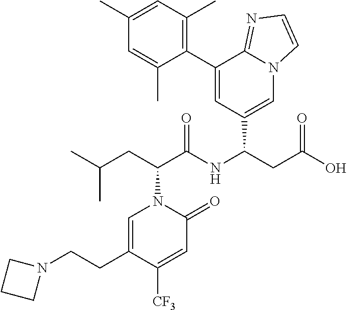 [SMILES: Cc1cc(C)c(-c2cc([C@H](CC(=O)O)NC(=O)[C@@H](CC(C)C)n3cc(CCN4CCC4)c(C(F)(F)F)cc3=O)cn3ccnc23)c(C)c1] | 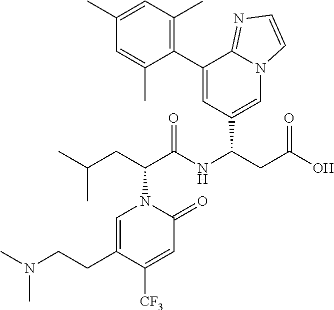 [SMILES: Cc1cc(C)c(-c2cc([C@H](CC(=O)O)NC(=O)[C@@H](CC(C)C)n3cc(CCN(C)C)c(C(F)(F)F)cc3=O)cn3ccnc23)c(C)c1] | 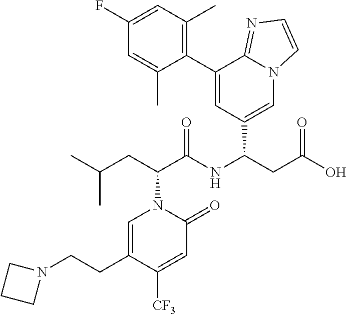 [SMILES: Cc1cc(F)cc(C)c1-c1cc([C@H](CC(=O)O)NC(=O)[C@@H](CC(C)C)n2cc(CCN3CCC3)c(C(F)(F)F)cc2=O)cn2ccnc12] |
| 104 | 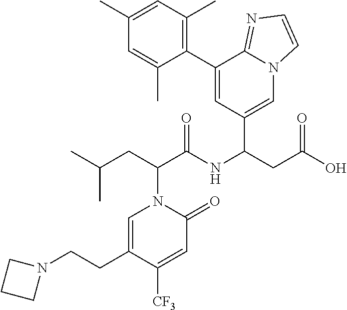 [SMILES: Cc1cc(C)c(-c2cc(C(CC(=O)O)NC(=O)C(CC(C)C)n3cc(CCN4CCC4)c(C(F)(F)F)cc3=O)cn3ccnc23)c(C)c1] | 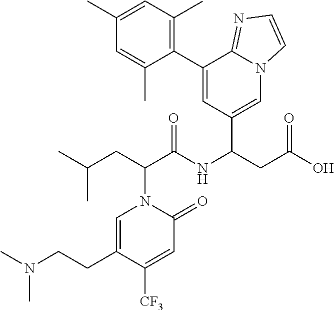 [SMILES: Cc1cc(C)c(-c2cc(C(CC(=O)O)NC(=O)C(CC(C)C)n3cc(CCN(C)C)c(C(F)(F)F)cc3=O)cn3ccnc23)c(C)c1] | 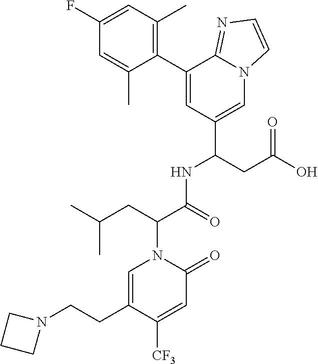 [SMILES: Cc1cc(F)cc(C)c1-c1cc(C(CC(=O)O)NC(=O)C(CC(C)C)n2cc(CCN3CCC3)c(C(F)(F)F)cc2=O)cn2ccnc12] |
| 105 | 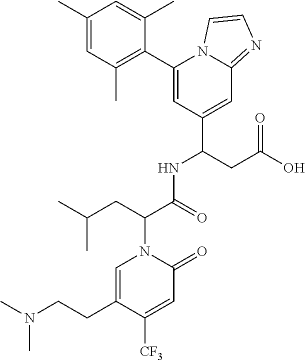 [SMILES: Cc1cc(C)c(-c2cc(C(CC(=O)O)NC(=O)C(CC(C)C)n3cc(CCN(C)C)c(C(F)(F)F)cc3=O)cc3nccn23)c(C)c1] | 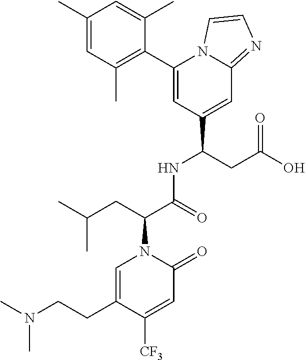 [SMILES: Cc1cc(C)c(-c2cc([C@@H](CC(=O)O)NC(=O)[C@H](CC(C)C)n3cc(CCN(C)C)c(C(F)(F)F)cc3=O)cc3nccn23)c(C)c1] | 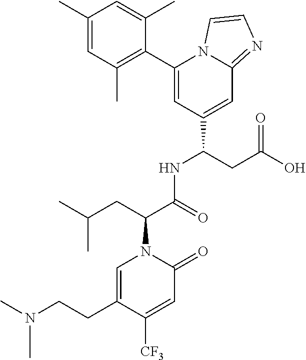 [SMILES: Cc1cc(C)c(-c2cc([C@H](CC(=O)O)NC(=O)[C@H](CC(C)C)n3cc(CCN(C)C)c(C(F)(F)F)cc3=O)cc3nccn23)c(C)c1] |
| 106 | 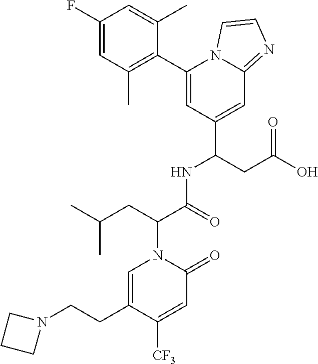 [SMILES: Cc1cc(F)cc(C)c1-c1cc(C(CC(=O)O)NC(=O)C(CC(C)C)n2cc(CCN3CCC3)c(C(F)(F)F)cc2=O)cc2nccn12] | 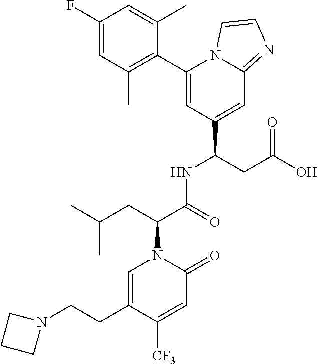 [SMILES: Cc1cc(F)cc(C)c1-c1cc([C@@H](CC(=O)O)NC(=O)[C@H](CC(C)C)n2cc(CCN3CCC3)c(C(F)(F)F)cc2=O)cc2nccn12] | 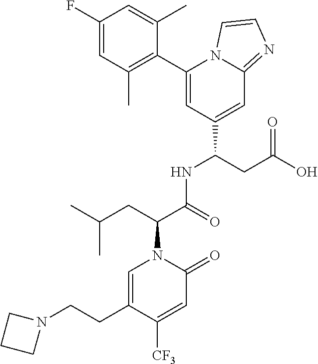 [SMILES: Cc1cc(F)cc(C)c1-c1cc([C@H](CC(=O)O)NC(=O)[C@H](CC(C)C)n2cc(CCN3CCC3)c(C(F)(F)F)cc2=O)cc2nccn12] |
| 107 | 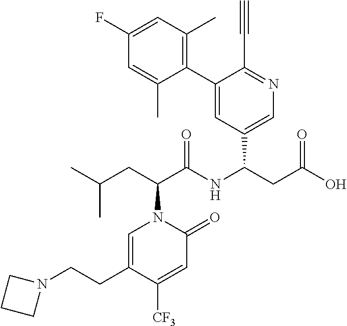 [SMILES: C#Cc1ncc([C@H](CC(=O)O)NC(=O)[C@H](CC(C)C)n2cc(CCN3CCC3)c(C(F)(F)F)cc2=O)cc1-c1c(C)cc(F)cc1C] | 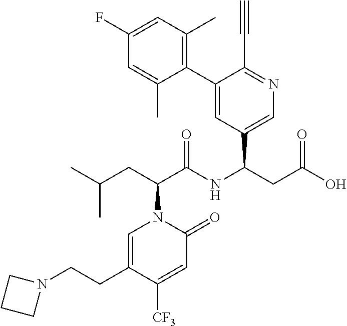 [SMILES: C#Cc1ncc([C@@H](CC(=O)O)NC(=O)[C@H](CC(C)C)n2cc(CCN3CCC3)c(C(F)(F)F)cc2=O)cc1-c1c(C)cc(F)cc1C] | 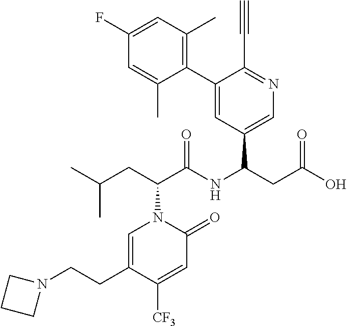 |
| 108 | 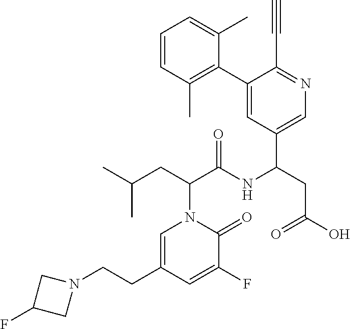 [SMILES: C#Cc1ncc(C(CC(=O)O)NC(=O)C(CC(C)C)n2cc(CCN3CC(F)C3)cc(F)c2=O)cc1-c1c(C)cccc1C] | 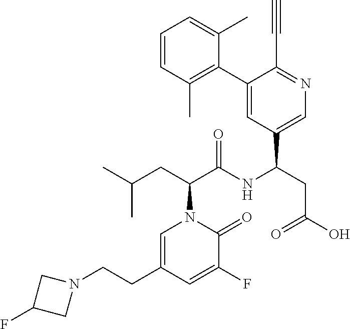 [SMILES: C#Cc1ncc([C@@H](CC(=O)O)NC(=O)[C@H](CC(C)C)n2cc(CCN3CC(F)C3)cc(F)c2=O)cc1-c1c(C)cccc1C] | 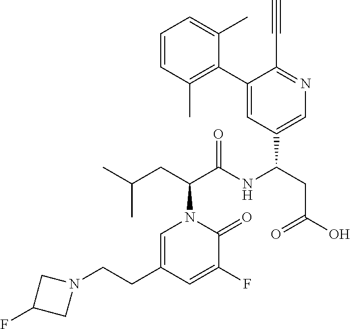 [SMILES: C#Cc1ncc([C@H](CC(=O)O)NC(=O)[C@H](CC(C)C)n2cc(CCN3CC(F)C3)cc(F)c2=O)cc1-c1c(C)cccc1C] |
| 109 | 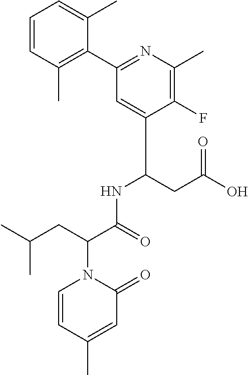 [SMILES: Cc1ccn(C(CC(C)C)C(=O)NC(CC(=O)O)c2cc(-c3c(C)cccc3C)nc(C)c2F)c(=O)c1] | 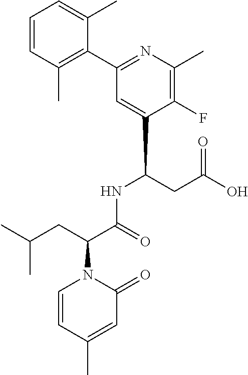 [SMILES: Cc1ccn([C@@H](CC(C)C)C(=O)N[C@H](CC(=O)O)c2cc(-c3c(C)cccc3C)nc(C)c2F)c(=O)c1] | 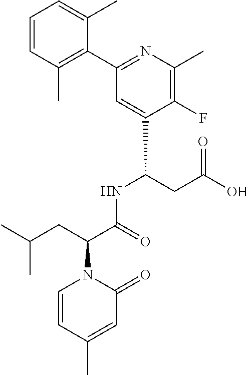 [SMILES: Cc1ccn([C@@H](CC(C)C)C(=O)N[C@@H](CC(=O)O)c2cc(-c3c(C)cccc3C)nc(C)c2F)c(=O)c1] |

| | | |
|---|---|---|
| 1 | 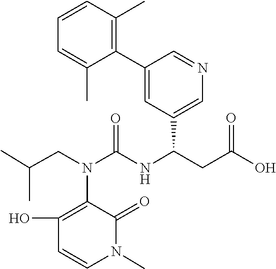 [SMILES: Cc1cccc(C)c1-c1cncc([C@H](CC(=O)O)NC(=O)N(CC(C)C)c2c(O)ccn(C)c2=O)c1] | 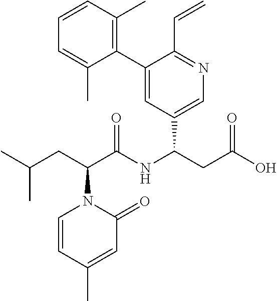 [SMILES: C=Cc1ncc([C@H](CC(=O)O)NC(=O)[C@H](CC(C)C)n2ccc(C)cc2=O)cc1-c1c(C)cccc1C] |
| 2 | 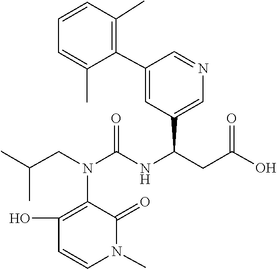 [SMILES: Cc1cccc(C)c1-c1cncc([C@@H](CC(=O)O)NC(=O)N(CC(C)C)c2c(O)ccn(C)c2=O)c1] | 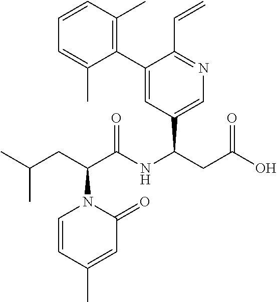 [SMILES: C=Cc1ncc([C@@H](CC(=O)O)NC(=O)[C@H](CC(C)C)n2ccc(C)cc2=O)cc1-c1c(C)cccc1C] |
| 3 | 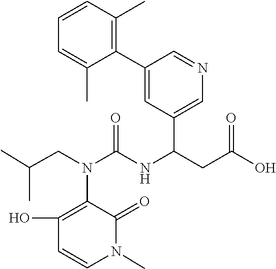 [SMILES: Cc1cccc(C)c1-c1cncc(C(CC(=O)O)NC(=O)N(CC(C)C)c2c(O)ccn(C)c2=O)c1] | 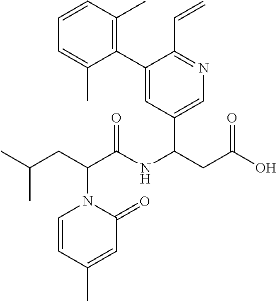 [SMILES: C=Cc1ncc(C(CC(=O)O)NC(=O)C(CC(C)C)n2ccc(C)cc2=O)cc1-c1c(C)cccc1C] |
| 4 | 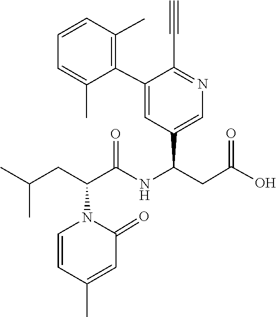 [SMILES: C#Cc1ncc([C@@H](CC(=O)O)NC(=O)[C@@H](CC(C)C)n2ccc(C)cc2=O)cc1-c1c(C)cccc1C] | 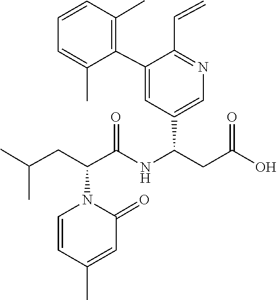 [SMILES: C=Cc1ncc([C@H](CC(=O)O)NC(=O)[C@@H](CC(C)C)n2ccc(C)cc2=O)cc1-c1c(C)cccc1C] |
| 5 | 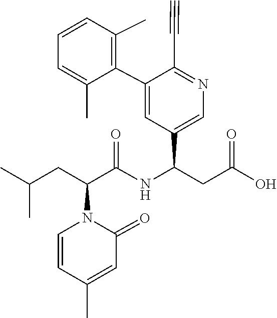 [SMILES: C#Cc1ncc([C@@H](CC(=O)O)NC(=O)[C@H](CC(C)C)n2ccc(C)cc2=O)cc1-c1c(C)cccc1C] | 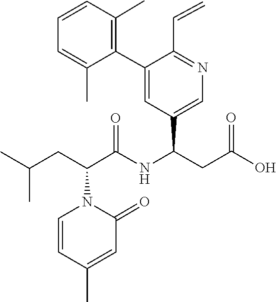 [SMILES: C=Cc1ncc([C@@H](CC(=O)O)NC(=O)[C@@H](CC(C)C)n2ccc(C)cc2=O)cc1-c1c(C)cccc1C] |
| 7 | 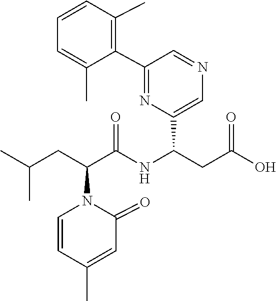 [SMILES: Cc1ccn([C@@H](CC(C)C)C(=O)N[C@@H](CC(=O)O)c2cncc(-c3c(C)cccc3C)n2)c(=O)c1] | 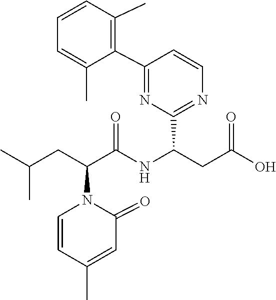 [SMILES: Cc1ccn([C@@H](CC(C)C)C(=O)N[C@@H](CC(=O)O)c2nccc(-c3c(C)cccc3C)n2)c(=O)c1] |
| 8 | 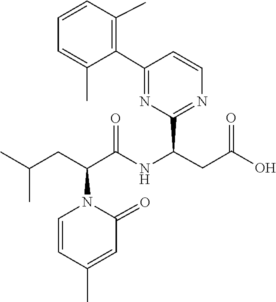 [SMILES: Cc1ccn([C@@H](CC(C)C)C(=O)N[C@H](CC(=O)O)c2nccc(-c3c(C)cccc3C)n2)c(=O)c1] | 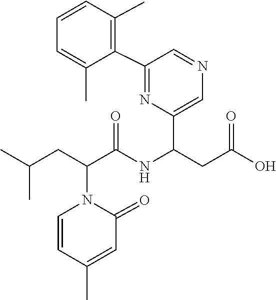 [SMILES: Cc1ccn(C(CC(C)C)C(=O)NC(CC(=O)O)c2cncc(-c3c(C)cccc3C)n2)c(=O)c1] |
| 9 | 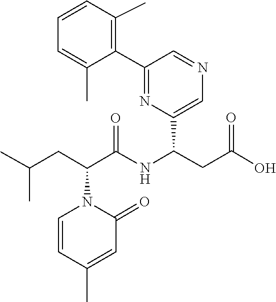 [SMILES: Cc1ccn([C@H](CC(C)C)C(=O)N[C@@H](CC(=O)O)c2cncc(-c3c(C)cccc3C)n2)c(=O)c1] | 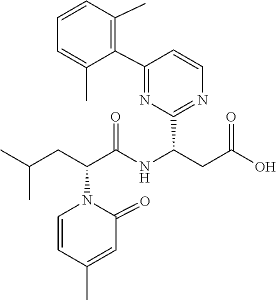 [SMILES: Cc1ccn([C@H](CC(C)C)C(=O)N[C@@H](CC(=O)O)c2nccc(-c3c(C)cccc3C)n2)c(=O)c1] |
| 10 | 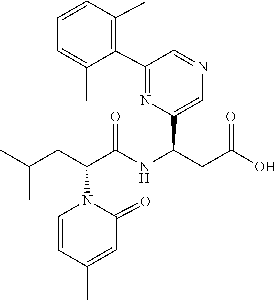 [SMILES: Cc1ccn([C@H](CC(C)C)C(=O)N[C@H](CC(=O)O)c2cncc(-c3c(C)cccc3C)n2)c(=O)c1] | 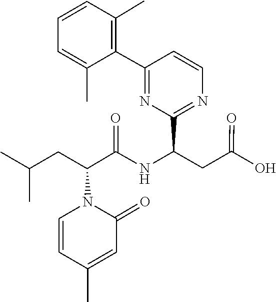 [SMILES: Cc1ccn([C@H](CC(C)C)C(=O)N[C@H](CC(=O)O)c2nccc(-c3c(C)cccc3C)n2)c(=O)c1] |
| 11 | 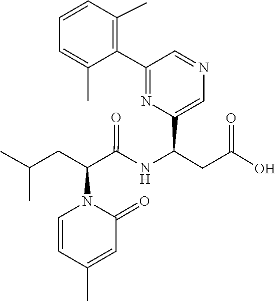 [SMILES: Cc1ccn([C@@H](CC(C)C)C(=O)N[C@H](CC(=O)O)c2cncc(-c3c(C)cccc3C)n2)c(=O)c1] | 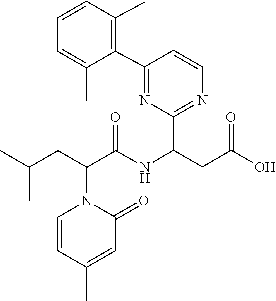 [SMILES: Cc1ccn(C(CC(C)C)C(=O)NC(CC(=O)O)c2nccc(-c3c(C)cccc3C)n2)c(=O)c1] |
| 12 | 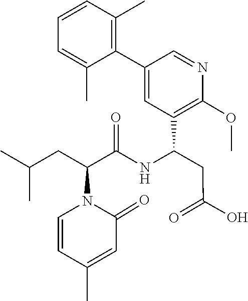 [SMILES: COc1ncc(-c2c(C)cccc2C)cc1[C@H](CC(=O)O)NC(=O)[C@H](CC(C)C)n1ccc(C)cc1=O] | 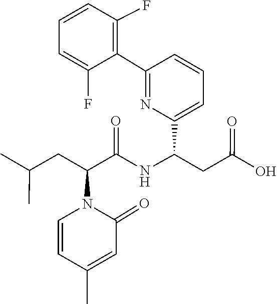 [SMILES: Cc1ccn([C@@H](CC(C)C)C(=O)N[C@@H](CC(=O)O)c2cccc(-c3c(F)cccc3F)n2)c(=O)c1] |
| 13 | 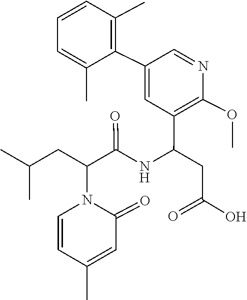 [SMILES: COc1ncc(-c2c(C)cccc2C)cc1C(CC(=O)O)NC(=O)C(CC(C)C)n1ccc(C)cc1=O] | 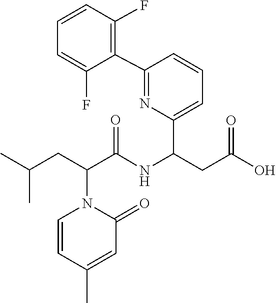 [SMILES: Cc1ccn(C(CC(C)C)C(=O)NC(CC(=O)O)c2cccc(-c3c(F)cccc3F)n2)c(=O)c1] |
| 14 | 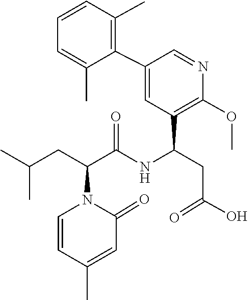 [SMILES: COc1ncc(-c2c(C)cccc2C)cc1[C@@H](CC(=O)O)NC(=O)[C@H](CC(C)C)n1ccc(C)cc1=O] | 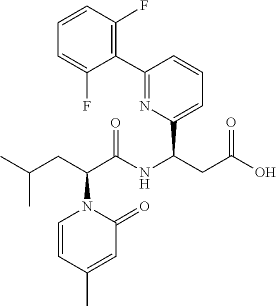 [SMILES: Cc1ccn([C@@H](CC(C)C)C(=O)N[C@H](CC(=O)O)c2cccc(-c3c(F)cccc3F)n2)c(=O)c1] |
| 15 | 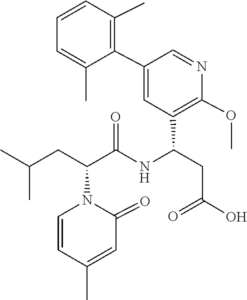 [SMILES: COc1ncc(-c2c(C)cccc2C)cc1[C@H](CC(=O)O)NC(=O)[C@@H](CC(C)C)n1ccc(C)cc1=O] | 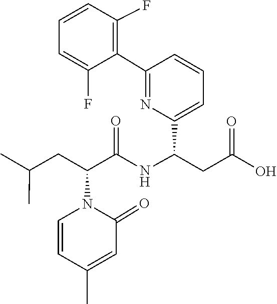 [SMILES: Cc1ccn([C@H](CC(C)C)C(=O)N[C@@H](CC(=O)O)c2cccc(-c3c(F)cccc3F)n2)c(=O)c1] |
| 16 | 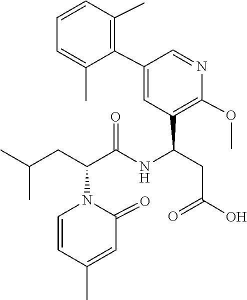 [SMILES: COc1ncc(-c2c(C)cccc2C)cc1[C@@H](CC(=O)O)NC(=O)[C@@H](CC(C)C)n1ccc(C)cc1=O] | 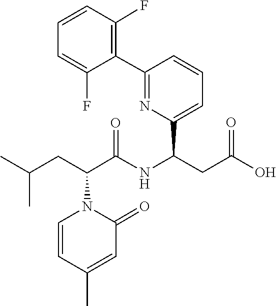 [SMILES: Cc1ccn([C@H](CC(C)C)C(=O)N[C@H](CC(=O)O)c2cccc(-c3c(F)cccc3F)n2)c(=O)c1] |
| 17 | 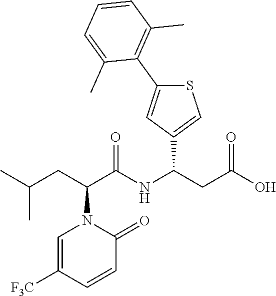 [SMILES: Cc1cccc(C)c1-c1cc([C@H](CC(=O)O)NC(=O)[C@H](CC(C)C)n2cc(C(F)(F)F)ccc2=O)cs1] | 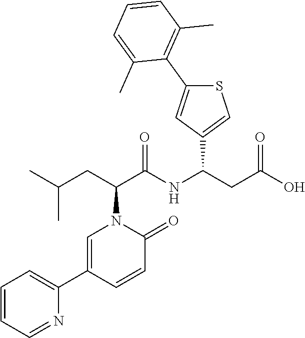 [SMILES: Cc1cccc(C)c1-c1cc([C@H](CC(=O)O)NC(=O)[C@H](CC(C)C)n2cc(-c3ccccn3)ccc2=O)cs1] |
| 18 | 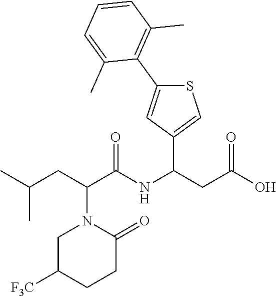 [SMILES: Cc1cccc(C)c1-c1cc(C(CC(=O)O)NC(=O)C(CC(C)C)N2CC(C(F)(F)F)CCC2=O)cs1] | 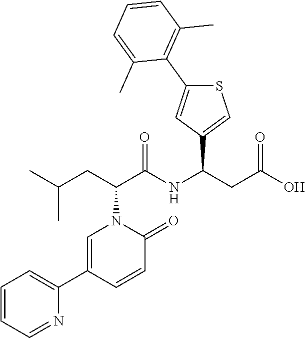 [SMILES: Cc1cccc(C)c1-c1cc([C@@H](CC(=O)O)NC(=O)[C@@H](CC(C)C)n2cc(-c3ccccn3)ccc2=O)cs1] |
| 19 | 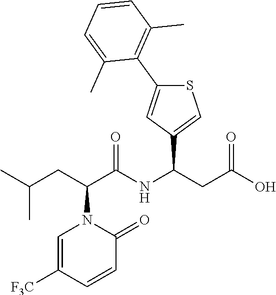 [SMILES: Cc1cccc(C)c1-c1cc([C@@H](CC(=O)O)NC(=O)[C@H](CC(C)C)n2cc(C(F)(F)F)ccc2=O)cs1] | 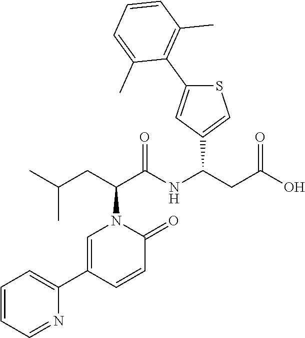 [SMILES: Cc1cccc(C)c1-c1cc([C@H](CC(=O)O)NC(=O)[C@H](CC(C)C)n2cc(-c3ccccn3)ccc2=O)cs1] |
| 20 | 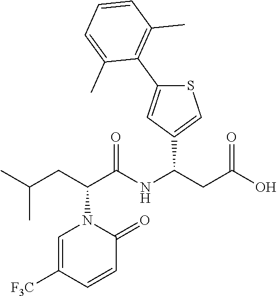 [SMILES: Cc1cccc(C)c1-c1cc([C@H](CC(=O)O)NC(=O)[C@@H](CC(C)C)n2cc(C(F)(F)F)ccc2=O)cs1] | 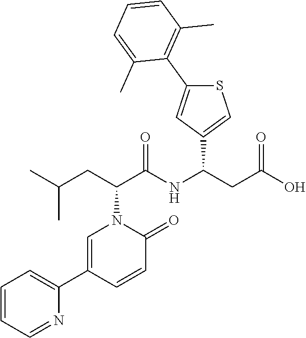 [SMILES: Cc1cccc(C)c1-c1cc([C@H](CC(=O)O)NC(=O)[C@@H](CC(C)C)n2cc(-c3ccccn3)ccc2=O)cs1] |
| 21 | 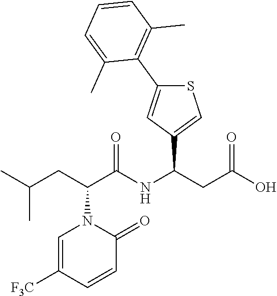 [SMILES: Cc1cccc(C)c1-c1cc([C@@H](CC(=O)O)NC(=O)[C@@H](CC(C)C)n2cc(C(F)(F)F)ccc2=O)cs1] | 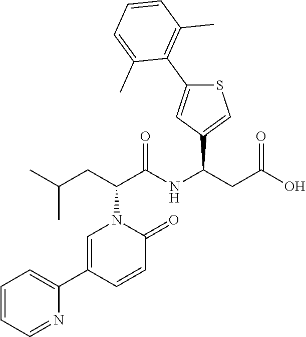 [SMILES: Cc1cccc(C)c1-c1cc([C@@H](CC(=O)O)NC(=O)[C@@H](CC(C)C)n2cc(-c3ccccn3)ccc2=O)cs1] |
| 22 | 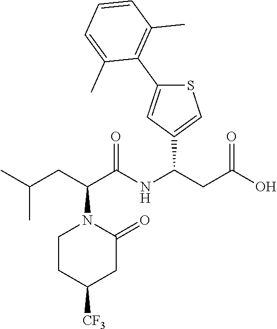 [SMILES: Cc1cccc(C)c1-c1cc([C@H](CC(=O)O)NC(=O)[C@H](CC(C)C)N2CC[C@H](C(F)(F)F)CC2=O)cs1] | 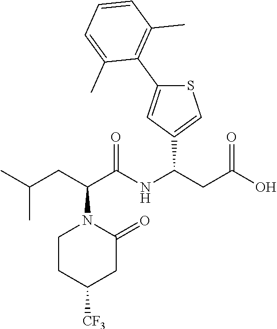 [SMILES: Cc1cccc(C)c1-c1cc([C@H](CC(=O)O)NC(=O)[C@H](CC(C)C)N2CC[C@@H](C(F)(F)F)CC2=O)cs1] |
| 23 | 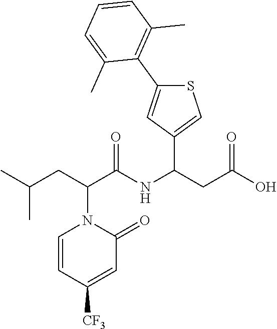 | 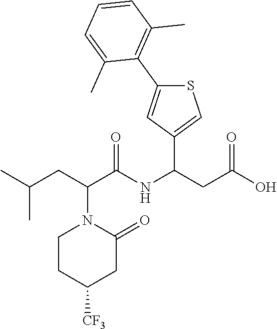 [SMILES: Cc1cccc(C)c1-c1cc(C(CC(=O)O)NC(=O)C(CC(C)C)N2CC[C@@H](C(F)(F)F)CC2=O)cs1] |
| 24 | 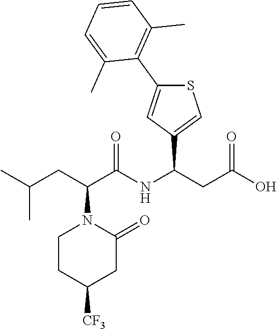 [SMILES: Cc1cccc(C)c1-c1cc([C@@H](CC(=O)O)NC(=O)[C@H](CC(C)C)N2CC[C@H](C(F)(F)F)CC2=O)cs1] | 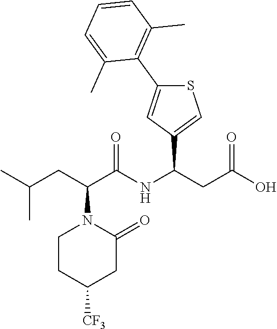 [SMILES: Cc1cccc(C)c1-c1cc([C@@H](CC(=O)O)NC(=O)[C@H](CC(C)C)N2CC[C@@H](C(F)(F)F)CC2=O)cs1] |
| 25 | 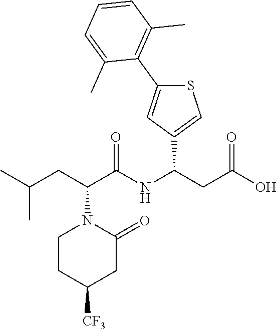 [SMILES: Cc1cccc(C)c1-c1cc([C@H](CC(=O)O)NC(=O)[C@@H](CC(C)C)N2CC[C@H](C(F)(F)F)CC2=O)cs1] | 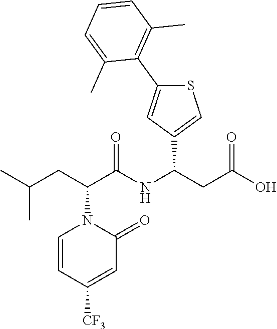 |
| 26 | 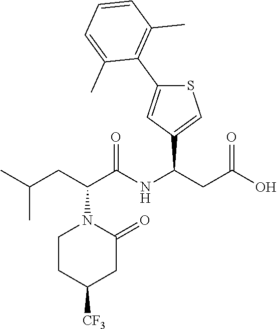 [SMILES: Cc1cccc(C)c1-c1cc([C@@H](CC(=O)O)NC(=O)[C@@H](CC(C)C)N2CC[C@H](C(F)(F)F)CC2=O)cs1] | 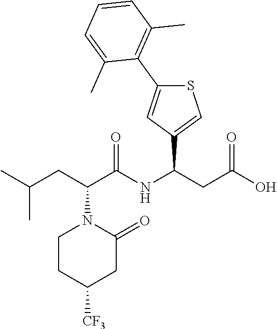 [SMILES: Cc1cccc(C)c1-c1cc([C@@H](CC(=O)O)NC(=O)[C@@H](CC(C)C)N2CC[C@@H](C(F)(F)F)CC2=O)cs1] |
| 27 | 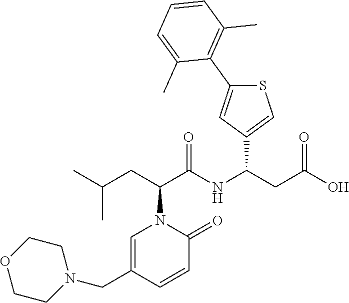 [SMILES: Cc1cccc(C)c1-c1cc([C@H](CC(=O)O)NC(=O)[C@H](CC(C)C)n2cc(CN3CCOCC3)ccc2=O)cs1] | 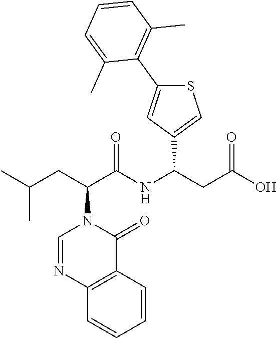 [SMILES: Cc1cccc(C)c1-c1cc([C@H](CC(=O)O)NC(=O)[C@H](CC(C)C)n2cnc3ccccc3c2=O)cs1] |
| 28 | 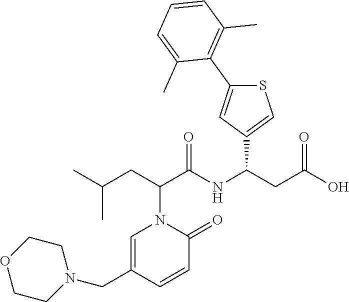 [SMILES: Cc1cccc(C)c1-c1cc([C@H](CC(=O)O)NC(=O)C(CC(C)C)n2cc(CN3CCOCC3)ccc2=O)cs1] | 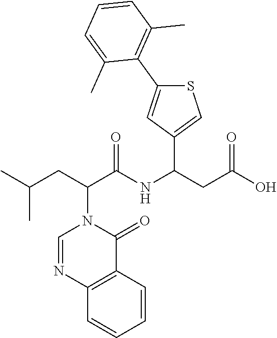 [SMILES: Cc1cccc(C)c1-c1cc(C(CC(=O)O)NC(=O)C(CC(C)C)n2cnc3ccccc3c2=O)cs1] |
| 29 | 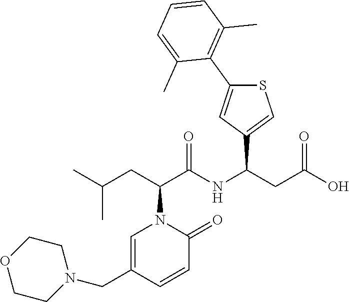 [SMILES: Cc1cccc(C)c1-c1cc([C@@H](CC(=O)O)NC(=O)[C@H](CC(C)C)n2cc(CN3CCOCC3)ccc2=O)cs1] | 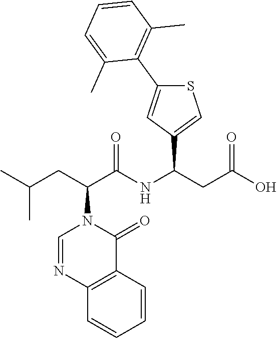 [SMILES: Cc1cccc(C)c1-c1cc([C@@H](CC(=O)O)NC(=O)[C@H](CC(C)C)n2cnc3ccccc3c2=O)cs1] |
| 30 | 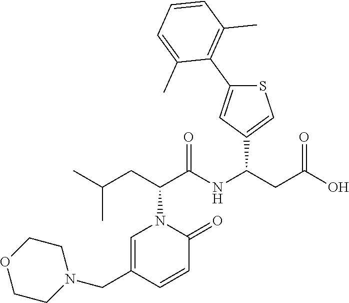 [SMILES: Cc1cccc(C)c1-c1cc([C@H](CC(=O)O)NC(=O)[C@@H](CC(C)C)n2cc(CN3CCOCC3)ccc2=O)cs1] | 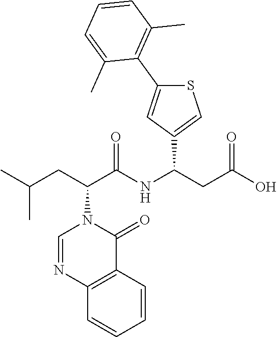 [SMILES: Cc1cccc(C)c1-c1cc([C@H](CC(=O)O)NC(=O)[C@@H](CC(C)C)n2cnc3ccccc3c2=O)cs1] |
| 31 | 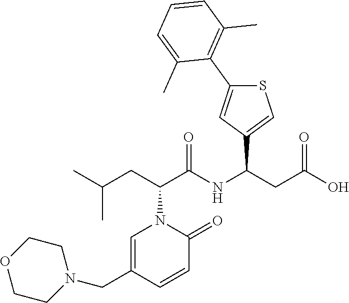 [SMILES: Cc1cccc(C)c1-c1cc([C@@H](CC(=O)O)NC(=O)[C@@H](CC(C)C)n2cc(CN3CCOCC3)ccc2=O)cs1] | 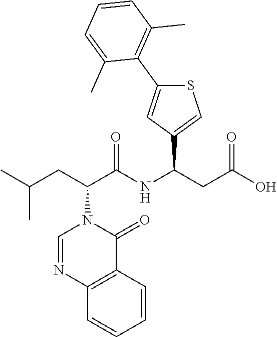 [SMILES: Cc1cccc(C)c1-c1cc([C@@H](CC(=O)O)NC(=O)[C@@H](CC(C)C)n2cnc3ccccc3c2=O)cs1] |
| 32 | 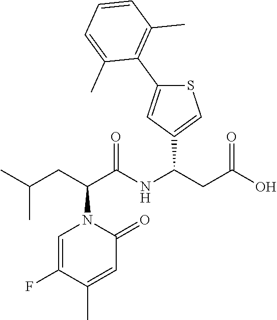 [SMILES: Cc1cc(=O)n([C@@H](CC(C)C)C(=O)N[C@@H](CC(=O)O)c2csc(-c3c(C)cccc3C)c2)cc1F] | 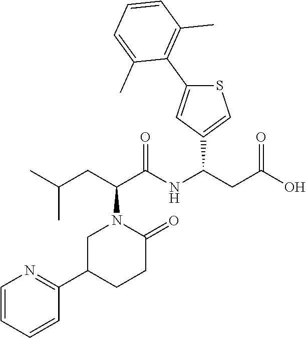 [SMILES: Cc1cccc(C)c1-c1cc([C@H](CC(=O)O)NC(=O)[C@H](CC(C)C)N2CC(c3ccccn3)CCC2=O)cs1] |
| 33 | 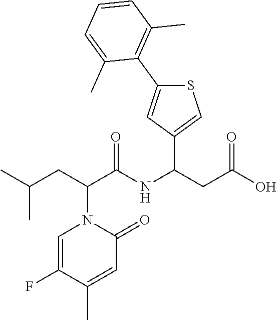 [SMILES: Cc1cc(=O)n(C(CC(C)C)C(=O)NC(CC(=O)O)c2csc(-c3c(C)cccc3C)c2)cc1F] | 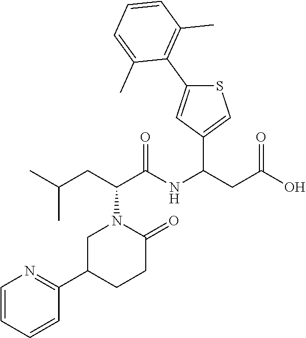 [SMILES: Cc1cccc(C)c1-c1cc(C(CC(=O)O)NC(=O)[C@@H](CC(C)C)N2CC(c3ccccn3)CCC2=O)cs1] |
| 34 | 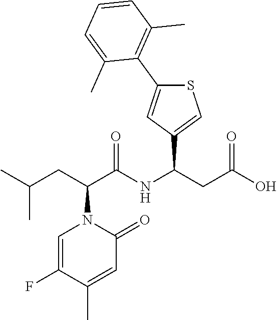 [SMILES: Cc1cc(=O)n([C@@H](CC(C)C)C(=O)N[C@H](CC(=O)O)c2csc(-c3c(C)cccc3C)c2)cc1F] | 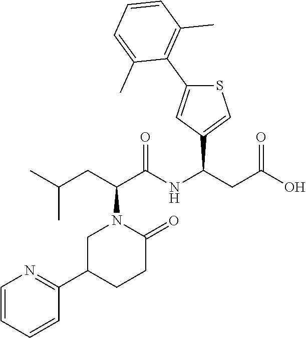 [SMILES: Cc1cccc(C)c1-c1cc([C@@H](CC(=O)O)NC(=O)[C@H](CC(C)C)N2CC(c3ccccn3)CCC2=O)cs1] |
| 35 | 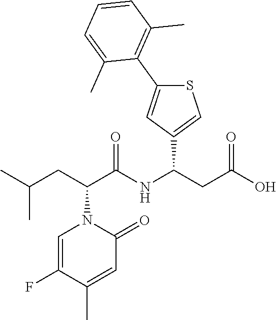 [SMILES: Cc1cc(=O)n([C@H](CC(C)C)C(=O)N[C@@H](CC(=O)O)c2csc(-c3c(C)cccc3C)c2)cc1F] | 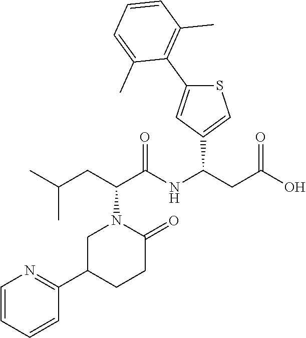 [SMILES: Cc1cccc(C)c1-c1cc([C@H](CC(=O)O)NC(=O)[C@@H](CC(C)C)N2CC(c3ccccn3)CCC2=O)cs1] |
| 36 | 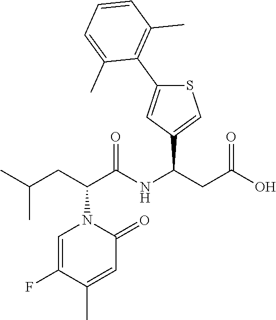 [SMILES: Cc1cc(=O)n([C@H](CC(C)C)C(=O)N[C@H](CC(=O)O)c2csc(-c3c(C)cccc3C)c2)cc1F] | 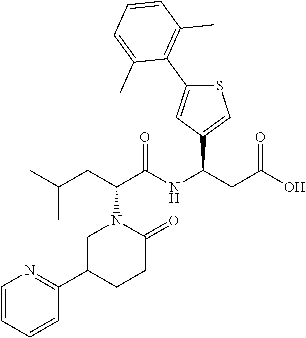 [SMILES: Cc1cccc(C)c1-c1cc([C@@H](CC(=O)O)NC(=O)[C@@H](CC(C)C)N2CC(c3ccccn3)CCC2=O)cs1] |
| 37 | 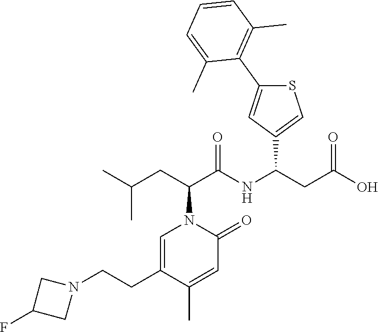 [SMILES: Cc1cc(=O)n([C@@H](CC(C)C)C(=O)N[C@@H](CC(=O)O)c2csc(-c3c(C)cccc3C)c2)cc1CCN1CC(F)C1] | 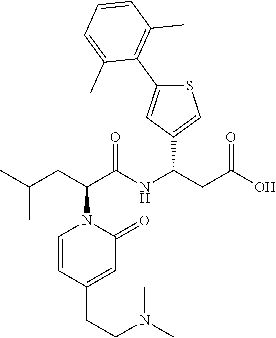 [SMILES: Cc1cccc(C)c1-c1cc([C@H](CC(=O)O)NC(=O)[C@H](CC(C)C)n2ccc(CCN(C)C)cc2=O)cs1] |
| 38 | 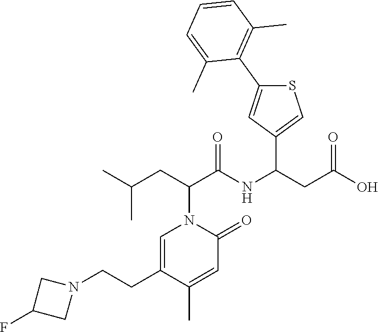 [SMILES: Cc1cc(=O)n(C(CC(C)C)C(=O)NC(CC(=O)O)c2csc(-c3c(C)cccc3C)c2)cc1CCN1CC(F)C1] | 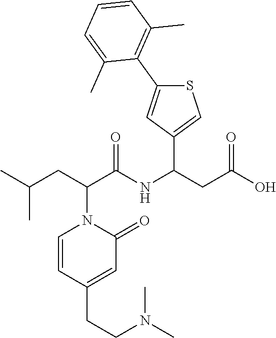 [SMILES: Cc1cccc(C)c1-c1cc(C(CC(=O)O)NC(=O)C(CC(C)C)n2ccc(CCN(C)C)cc2=O)cs1] |
| 39 | 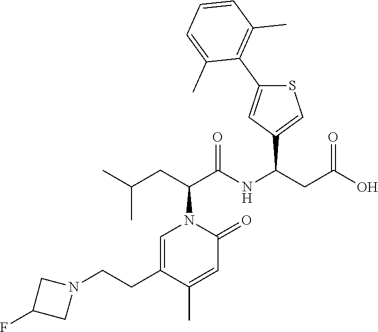 [SMILES: Cc1cc(=O)n([C@@H](CC(C)C)C(=O)N[C@H](CC(=O)O)c2csc(-c3c(C)cccc3C)c2)cc1CCN1CC(F)C1] | 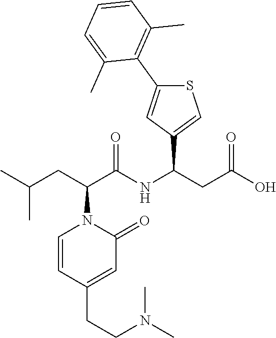 [SMILES: Cc1cccc(C)c1-c1cc([C@@H](CC(=O)O)NC(=O)[C@H](CC(C)C)n2ccc(CCN(C)C)cc2=O)cs1] |
| 40 | 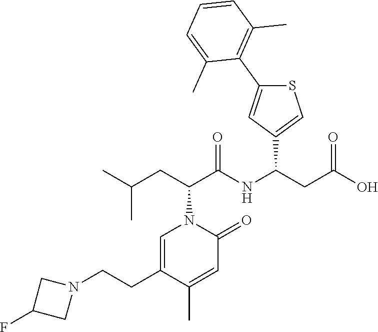 [SMILES: Cc1cc(=O)n([C@H](CC(C)C)C(=O)N[C@@H](CC(=O)O)c2csc(-c3c(C)cccc3C)c2)cc1CCN1CC(F)C1] | 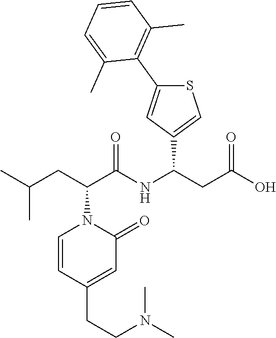 [SMILES: Cc1cccc(C)c1-c1cc([C@H](CC(=O)O)NC(=O)[C@@H](CC(C)C)n2ccc(CCN(C)C)cc2=O)cs1] |
| 41 | 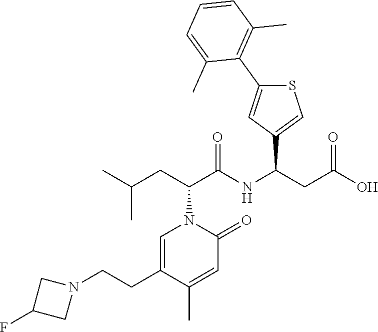 [SMILES: Cc1cc(=O)n([C@H](CC(C)C)C(=O)N[C@H](CC(=O)O)c2csc(-c3c(C)cccc3C)c2)cc1CCN1CC(F)C1] | 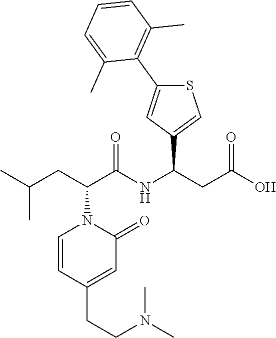 [SMILES: Cc1cccc(C)c1-c1cc([C@@H](CC(=O)O)NC(=O)[C@@H](CC(C)C)n2ccc(CCN(C)C)cc2=O)cs1] |
| 42 | 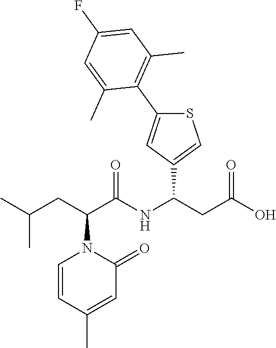 [SMILES: Cc1ccn([C@@H](CC(C)C)C(=O)N[C@@H](CC(=O)O)c2csc(-c3c(C)cc(F)cc3C)c2)c(=O)c1] | 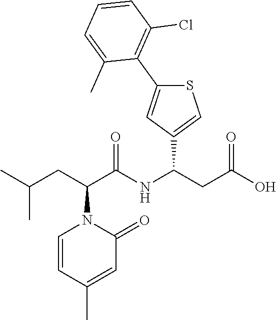 [SMILES: Cc1ccn([C@@H](CC(C)C)C(=O)N[C@@H](CC(=O)O)c2csc(-c3c(C)cccc3Cl)c2)c(=O)c1] |
| 43 | 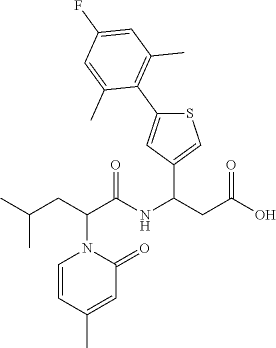 [SMILES: Cc1ccn(C(CC(C)C)C(=O)NC(CC(=O)O)c2csc(-c3c(C)cc(F)cc3C)c2)c(=O)c1] | 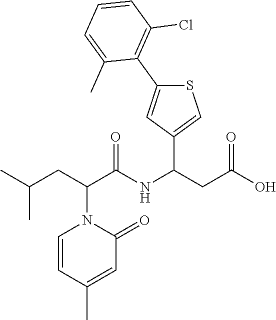 [SMILES: Cc1ccn(C(CC(C)C)C(=O)NC(CC(=O)O)c2csc(-c3c(C)cccc3Cl)c2)c(=O)c1] |
| 44 | 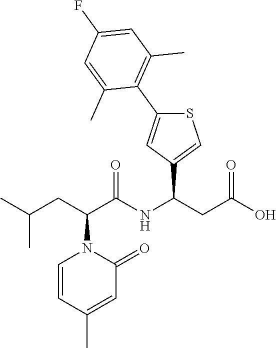 [SMILES: Cc1ccn([C@@H](CC(C)C)C(=O)N[C@H](CC(=O)O)c2csc(-c3c(C)cc(F)cc3C)c2)c(=O)c1] | 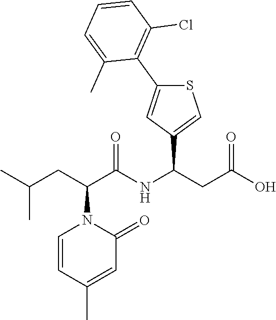 [SMILES: Cc1ccn([C@@H](CC(C)C)C(=O)N[C@H](CC(=O)O)c2csc(-c3c(C)cccc3Cl)c2)c(=O)c1] |
| 45 | 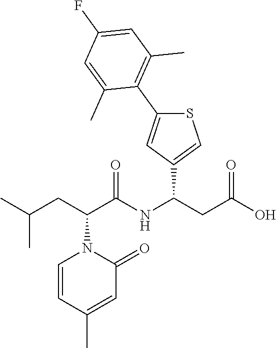 [SMILES: Cc1ccn([C@H](CC(C)C)C(=O)N[C@@H](CC(=O)O)c2csc(-c3c(C)cc(F)cc3C)c2)c(=O)c1] | 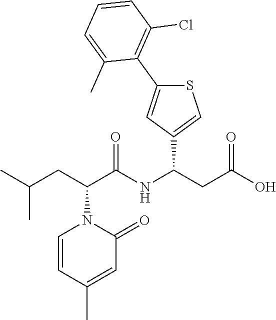 [SMILES: Cc1ccn([C@H](CC(C)C)C(=O)N[C@@H](CC(=O)O)c2csc(-c3c(C)cccc3Cl)c2)c(=O)c1] |
| 46 | 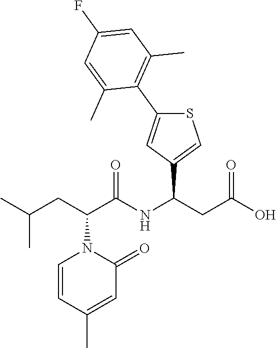 [SMILES: Cc1ccn([C@H](CC(C)C)C(=O)N[C@H](CC(=O)O)c2csc(-c3c(C)cc(F)cc3C)c2)c(=O)c1] | 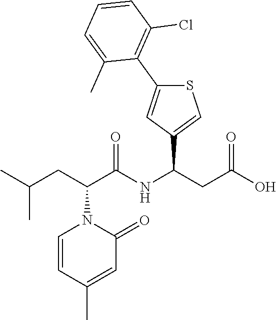 [SMILES: Cc1ccn([C@H](CC(C)C)C(=O)N[C@H](CC(=O)O)c2csc(-c3c(C)cccc3Cl)c2)c(=O)c1] |
| 47 | 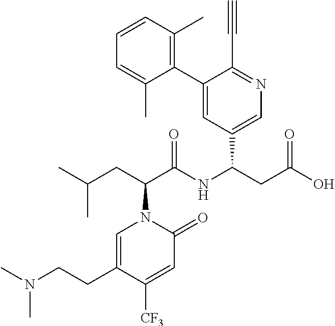 [SMILES: C#Cc1ncc([C@H](CC(=O)O)NC(=O)[C@H](CC(C)C)n2cc(CCN(C)C)c(C(F)(F)F)cc2=O)cc1-c1c(C)cccc1C] | 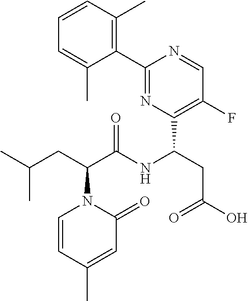 [SMILES: Cc1ccn([C@@H](CC(C)C)C(=O)N[C@@H](CC(=O)O)c2nc(-c3c(C)cccc3C)ncc2F)c(=O)c1] |
| 48 | 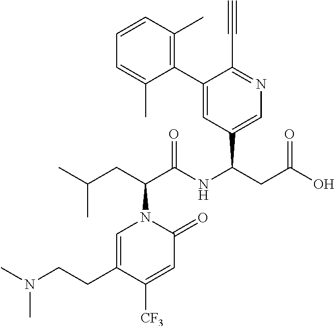 [SMILES: C#Cc1ncc([C@@H](CC(=O)O)NC(=O)[C@H](CC(C)C)n2cc(CCN(C)C)c(C(F)(F)F)cc2=O)cc1-c1c(C)cccc1C] | 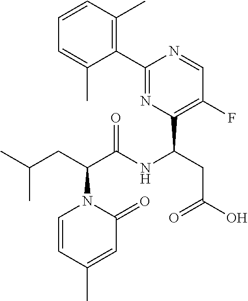 [SMILES: Cc1ccn([C@@H](CC(C)C)C(=O)N[C@H](CC(=O)O)c2nc(-c3c(C)cccc3C)ncc2F)c(=O)c1] |
| 49 | 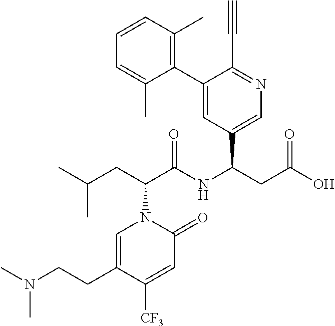 [SMILES: C#Cc1ncc([C@@H](CC(=O)O)NC(=O)[C@@H](CC(C)C)n2cc(CCN(C)C)c(C(F)(F)F)cc2=O)cc1-c1c(C)cccc1C] | 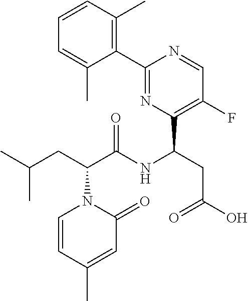 [SMILES: Cc1ccn([C@H](CC(C)C)C(=O)N[C@H](CC(=O)O)c2nc(-c3c(C)cccc3C)ncc2F)c(=O)c1] |
| 50 | 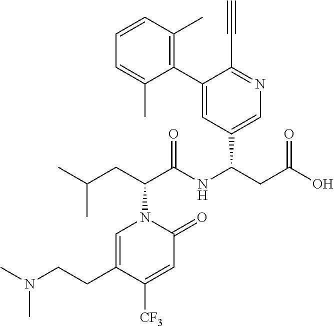 [SMILES: C#Cc1ncc([C@H](CC(=O)O)NC(=O)[C@@H](CC(C)C)n2cc(CCN(C)C)c(C(F)(F)F)cc2=O)cc1-c1c(C)cccc1C] | 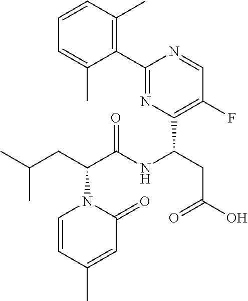 [SMILES: Cc1ccn([C@H](CC(C)C)C(=O)N[C@@H](CC(=O)O)c2nc(-c3c(C)cccc3C)ncc2F)c(=O)c1] |
| 51 | 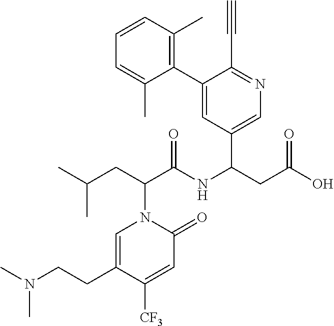 [SMILES: C#Cc1ncc(C(CC(=O)O)NC(=O)C(CC(C)C)n2cc(CCN(C)C)c(C(F)(F)F)cc2=O)cc1-c1c(C)cccc1C] | 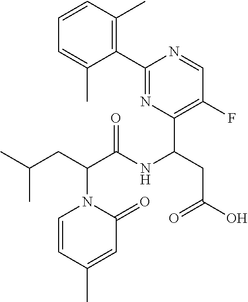 [SMILES: Cc1ccn(C(CC(C)C)C(=O)NC(CC(=O)O)c2nc(-c3c(C)cccc3C)ncc2F)c(=O)c1] |
| 52 | 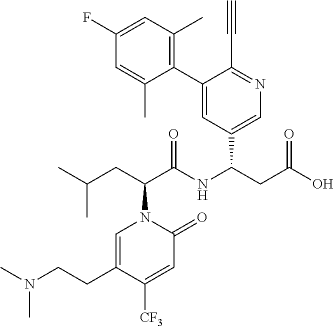 [SMILES: C#Cc1ncc([C@H](CC(=O)O)NC(=O)[C@H](CC(C)C)n2cc(CCN(C)C)c(C(F)(F)F)cc2=O)cc1-c1c(C)cc(F)cc1C] | 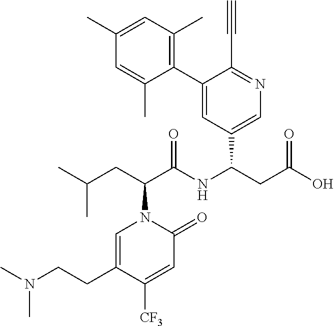 [SMILES: C#Cc1ncc([C@H](CC(=O)O)NC(=O)[C@H](CC(C)C)n2cc(CCN(C)C)c(C(F)(F)F)cc2=O)cc1-c1c(C)cc(C)cc1C] |
| 53 | 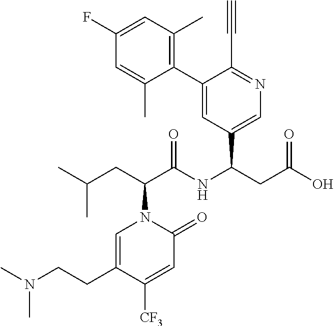 [SMILES: C#Cc1ncc([C@@H](CC(=O)O)NC(=O)[C@H](CC(C)C)n2cc(CCN(C)C)c(C(F)(F)F)cc2=O)cc1-c1c(C)cc(F)cc1C] | 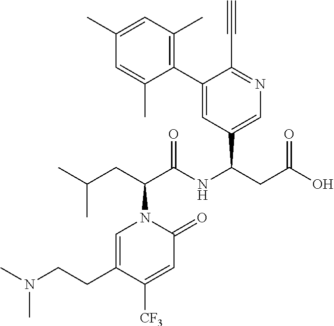 [SMILES: C#Cc1ncc([C@@H](CC(=O)O)NC(=O)[C@H](CC(C)C)n2cc(CCN(C)C)c(C(F)(F)F)cc2=O)cc1-c1c(C)cc(C)cc1C] |
| 54 | 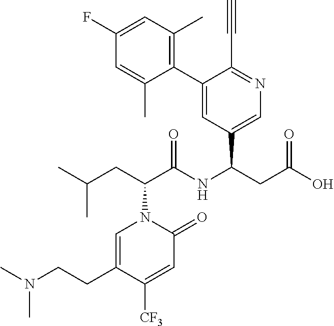 [SMILES: C#Cc1ncc([C@@H](CC(=O)O)NC(=O)[C@@H](CC(C)C)n2cc(CCN(C)C)c(C(F)(F)F)cc2=O)cc1-c1c(C)cc(F)cc1C] | 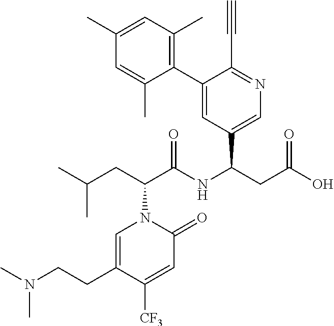 [SMILES: C#Cc1ncc([C@@H](CC(=O)O)NC(=O)[C@@H](CC(C)C)n2cc(CCN(C)C)c(C(F)(F)F)cc2=O)cc1-c1c(C)cc(C)cc1C] |
| 55 | 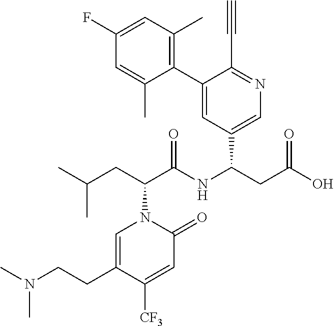 [SMILES: C#Cc1ncc([C@H](CC(=O)O)NC(=O)[C@@H](CC(C)C)n2cc(CCN(C)C)c(C(F)(F)F)cc2=O)cc1-c1c(C)cc(F)cc1C] | 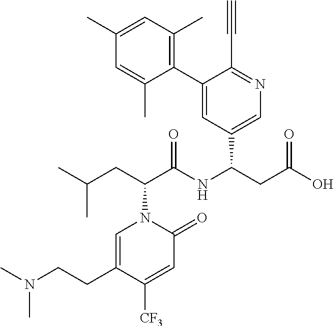 [SMILES: C#Cc1ncc([C@H](CC(=O)O)NC(=O)[C@@H](CC(C)C)n2cc(CCN(C)C)c(C(F)(F)F)cc2=O)cc1-c1c(C)cc(C)cc1C] |
| 56 | 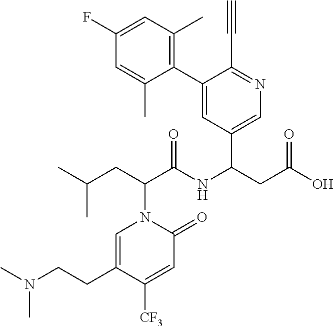 [SMILES: C#Cc1ncc(C(CC(=O)O)NC(=O)C(CC(C)C)n2cc(CCN(C)C)c(C(F)(F)F)cc2=O)cc1-c1c(C)cc(F)cc1C] | 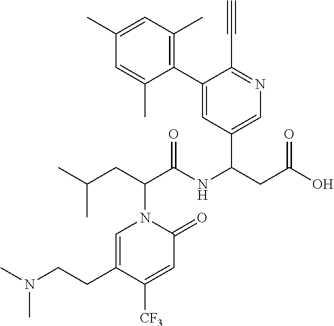 [SMILES: C#Cc1ncc(C(CC(=O)O)NC(=O)C(CC(C)C)n2cc(CCN(C)C)c(C(F)(F)F)cc2=O)cc1-c1c(C)cc(C)cc1C] |
| 57 | 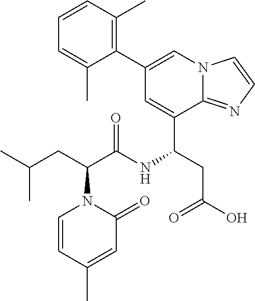 [SMILES: Cc1ccn([C@@H](CC(C)C)C(=O)N[C@@H](CC(=O)O)c2cc(-c3c(C)cccc3C)cn3ccnc23)c(=O)c1] | 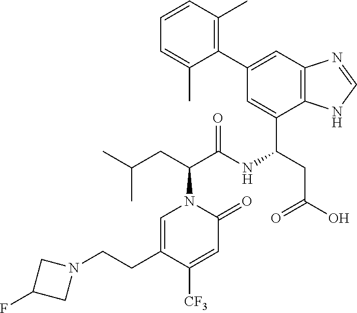 [SMILES: Cc1cccc(C)c1-c1cc([C@H](CC(=O)O)NC(=O)[C@H](CC(C)C)n2cc(CCN3CC(F)C3)c(C(F)(F)F)cc2=O)c2[nH]cnc2c1] |
| 58 | 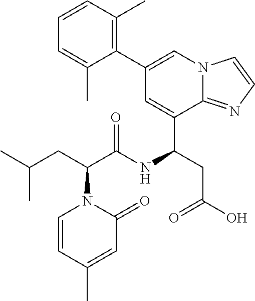 [SMILES: Cc1ccn([C@@H](CC(C)C)C(=O)N[C@H](CC(=O)O)c2cc(-c3c(C)cccc3C)cn3ccnc23)c(=O)c1] | 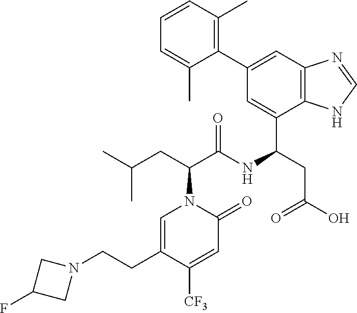 [SMILES: Cc1cccc(C)c1-c1cc([C@@H](CC(=O)O)NC(=O)[C@H](CC(C)C)n2cc(CCN3CC(F)C3)c(C(F)(F)F)cc2=O)c2[nH]cnc2c1] |
| 59 | 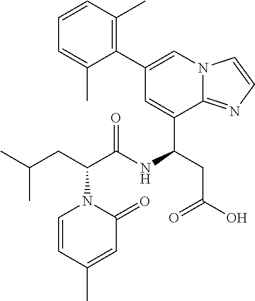 [SMILES: Cc1ccn([C@H](CC(C)C)C(=O)N[C@H](CC(=O)O)c2cc(-c3c(C)cccc3C)cn3ccnc23)c(=O)c1] | 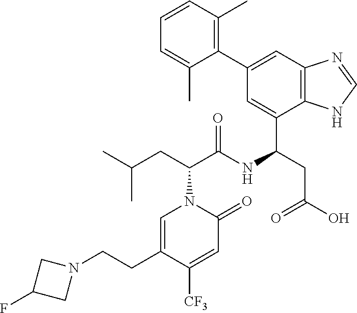 [SMILES: Cc1cccc(C)c1-c1cc([C@@H](CC(=O)O)NC(=O)[C@@H](CC(C)C)n2cc(CCN3CC(F)C3)c(C(F)(F)F)cc2=O)c2[nH]cnc2c1] |
| 60 | 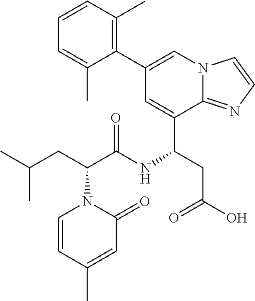 [SMILES: Cc1ccn([C@H](CC(C)C)C(=O)N[C@@H](CC(=O)O)c2cc(-c3c(C)cccc3C)cn3ccnc23)c(=O)c1] | 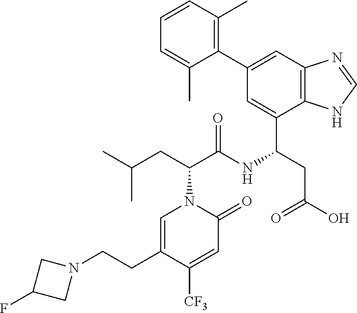 [SMILES: Cc1cccc(C)c1-c1cc([C@H](CC(=O)O)NC(=O)[C@@H](CC(C)C)n2cc(CCN3CC(F)C3)c(C(F)(F)F)cc2=O)c2[nH]cnc2c1] |
| 61 | 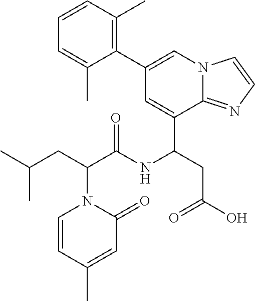 [SMILES: Cc1ccn(C(CC(C)C)C(=O)NC(CC(=O)O)c2cc(-c3c(C)cccc3C)cn3ccnc23)c(=O)c1] | 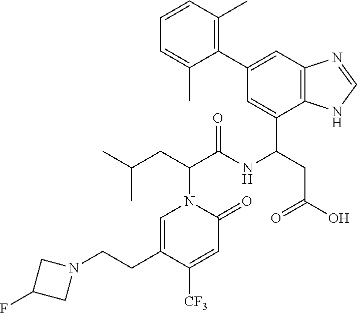 [SMILES: Cc1cccc(C)c1-c1cc(C(CC(=O)O)NC(=O)C(CC(C)C)n2cc(CCN3CC(F)C3)c(C(F)(F)F)cc2=O)c2[nH]cnc2c1] |
| 62 | 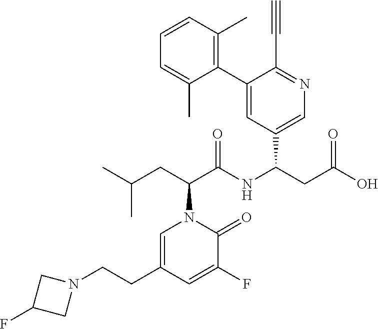 [SMILES: C#Cc1ncc([C@H](CC(=O)O)NC(=O)[C@H](CC(C)C)n2cc(CCN3CC(F)C3)cc(F)c2=O)cc1-c1c(C)cccc1C] | 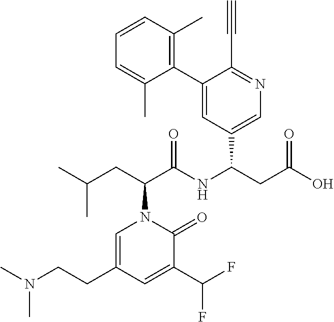 [SMILES: C#Cc1ncc([C@H](CC(=O)O)NC(=O)[C@H](CC(C)C)n2cc(CCN(C)C)cc(C(F)F)c2=O)cc1-c1c(C)cccc1C] |
| 63 | 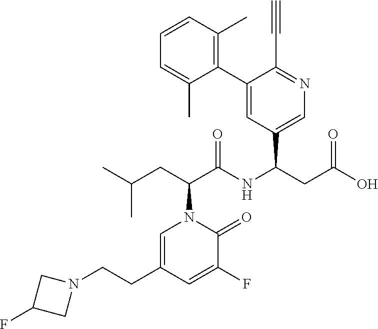 [SMILES: C#Cc1ncc([C@@H](CC(=O)O)NC(=O)[C@H](CC(C)C)n2cc(CCN3CC(F)C3)cc(F)c2=O)cc1-c1c(C)cccc1C] | 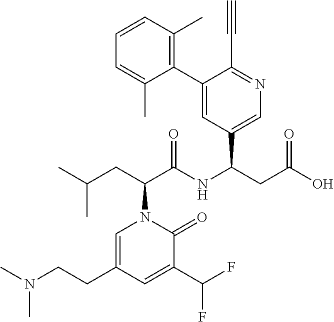 [SMILES: C#Cc1ncc([C@@H](CC(=O)O)NC(=O)[C@H](CC(C)C)n2cc(CCN(C)C)cc(C(F)F)c2=O)cc1-c1c(C)cccc1C] |
| 64 | 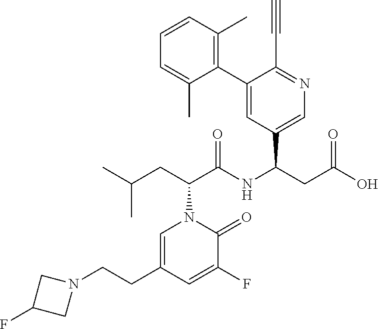 [SMILES: C#Cc1ncc([C@@H](CC(=O)O)NC(=O)[C@@H](CC(C)C)n2cc(CCN3CC(F)C3)cc(F)c2=O)cc1-c1c(C)cccc1C] | 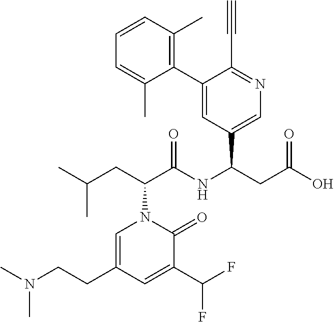 [SMILES: C#Cc1ncc([C@@H](CC(=O)O)NC(=O)[C@@H](CC(C)C)n2cc(CCN(C)C)cc(C(F)F)c2=O)cc1-c1c(C)cccc1C] |
| 65 | 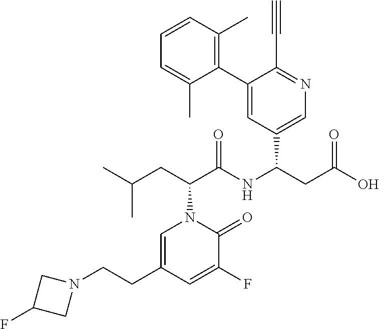 [SMILES: C#Cc1ncc([C@H](CC(=O)O)NC(=O)[C@@H](CC(C)C)n2cc(CCN3CC(F)C3)cc(F)c2=O)cc1-c1c(C)cccc1C] | 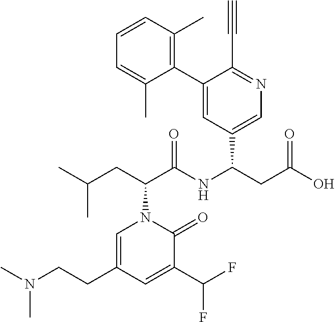 [SMILES: C#Cc1ncc([C@H](CC(=O)O)NC(=O)[C@@H](CC(C)C)n2cc(CCN(C)C)cc(C(F)F)c2=O)cc1-c1c(C)cccc1C] |
| 66 | 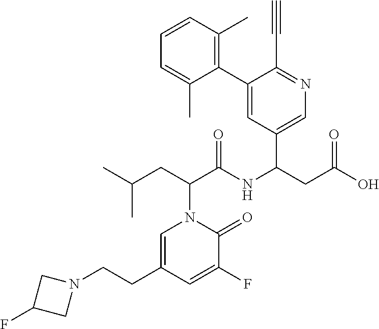 [SMILES: C#Cc1ncc(C(CC(=O)O)NC(=O)C(CC(C)C)n2cc(CCN3CC(F)C3)cc(F)c2=O)cc1-c1c(C)cccc1C] | 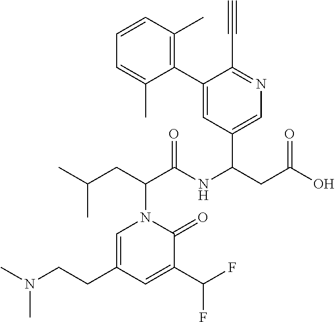 [SMILES: C#Cc1ncc(C(CC(=O)O)NC(=O)C(CC(C)C)n2cc(CCN(C)C)cc(C(F)F)c2=O)cc1-c1c(C)cccc1C] |
| 67 | 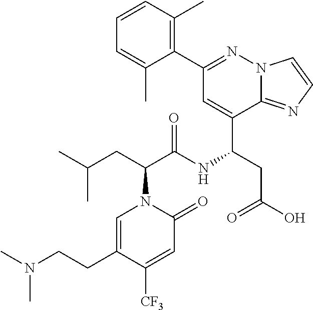 [SMILES: Cc1cccc(C)c1-c1cc([C@H](CC(=O)O)NC(=O)[C@H](CC(C)C)n2cc(CCN(C)C)c(C(F)(F)F)cc2=O)c2nccn2n1] | 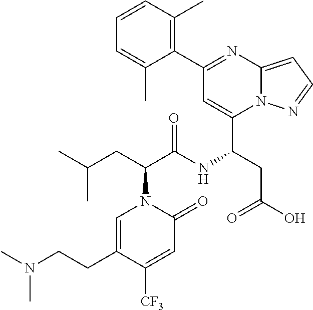 [SMILES: Cc1cccc(C)c1-c1cc([C@H](CC(=O)O)NC(=O)[C@H](CC(C)C)n2cc(CCN(C)C)c(C(F)(F)F)cc2=O)n2nccc2n1] |
| 68 | 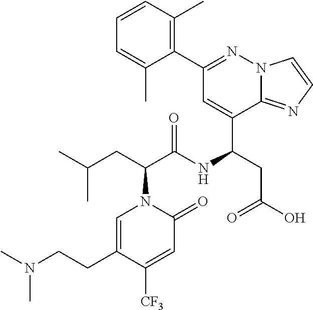 [SMILES: Cc1cccc(C)c1-c1cc([C@@H](CC(=O)O)NC(=O)[C@H](CC(C)C)n2cc(CCN(C)C)c(C(F)(F)F)cc2=O)c2nccn2n1] | 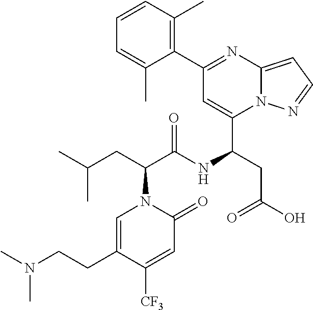 [SMILES: Cc1cccc(C)c1-c1cc([C@@H](CC(=O)O)NC(=O)[C@H](CC(C)C)n2cc(CCN(C)C)c(C(F)(F)F)cc2=O)n2nccc2n1] |
| 69 | 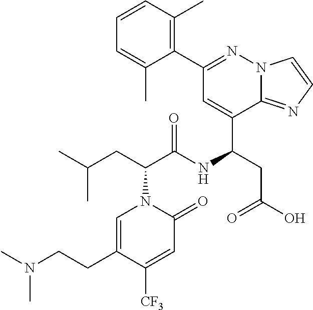 [SMILES: Cc1cccc(C)c1-c1cc([C@@H](CC(=O)O)NC(=O)[C@@H](CC(C)C)n2cc(CCN(C)C)c(C(F)(F)F)cc2=O)c2nccn2n1] | 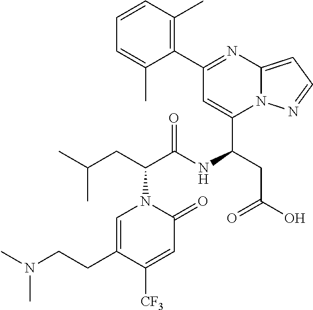 [SMILES: Cc1cccc(C)c1-c1cc([C@@H](CC(=O)O)NC(=O)[C@@H](CC(C)C)n2cc(CCN(C)C)c(C(F)(F)F)cc2=O)n2nccc2n1] |
| 70 | 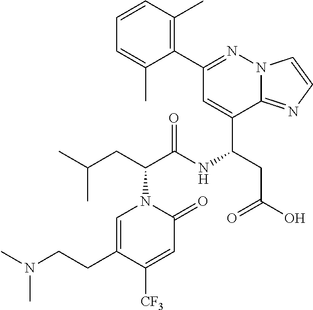 [SMILES: Cc1cccc(C)c1-c1cc([C@H](CC(=O)O)NC(=O)[C@@H](CC(C)C)n2cc(CCN(C)C)c(C(F)(F)F)cc2=O)c2nccn2n1] | 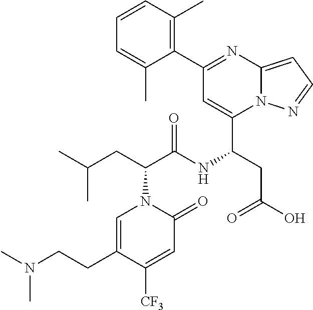 [SMILES: Cc1cccc(C)c1-c1cc([C@H](CC(=O)O)NC(=O)[C@@H](CC(C)C)n2cc(CCN(C)C)c(C(F)(F)F)cc2=O)n2nccc2n1] |
| 71 | 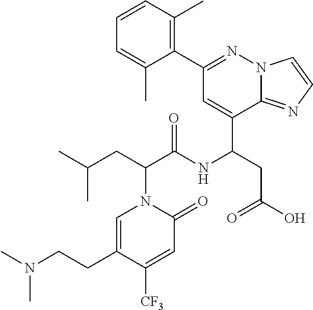 [SMILES: Cc1cccc(C)c1-c1cc(C(CC(=O)O)NC(=O)C(CC(C)C)n2cc(CCN(C)C)c(C(F)(F)F)cc2=O)c2nccn2n1] | 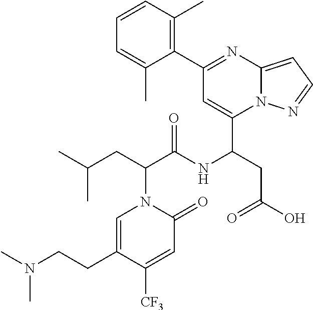 [SMILES: Cc1cccc(C)c1-c1cc(C(CC(=O)O)NC(=O)C(CC(C)C)n2cc(CCN(C)C)c(C(F)(F)F)cc2=O)n2nccc2n1] |
| 72 | 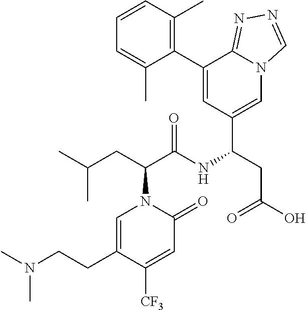 [SMILES: Cc1cccc(C)c1-c1cc([C@H](CC(=O)O)NC(=O)[C@H](CC(C)C)n2cc(CCN(C)C)c(C(F)(F)F)cc2=O)cn2cnnc12] | 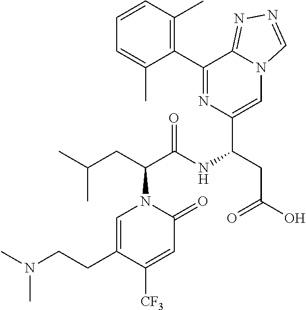 [SMILES: Cc1cccc(C)c1-c1nc([C@H](CC(=O)O)NC(=O)[C@H](CC(C)C)n2cc(CCN(C)C)c(C(F)(F)F)cc2=O)cn2cnnc12] |
| 73 | 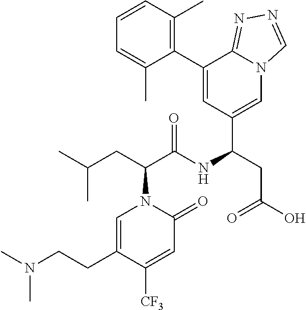 [SMILES: Cc1cccc(C)c1-c1cc([C@@H](CC(=O)O)NC(=O)[C@H](CC(C)C)n2cc(CCN(C)C)c(C(F)(F)F)cc2=O)cn2cnnc12] | 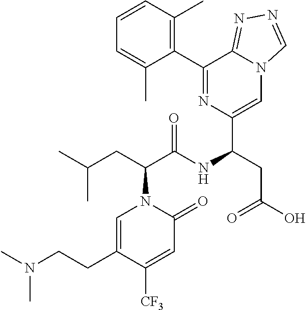 [SMILES: Cc1cccc(C)c1-c1nc([C@@H](CC(=O)O)NC(=O)[C@H](CC(C)C)n2cc(CCN(C)C)c(C(F)(F)F)cc2=O)cn2cnnc12] |
| 74 | 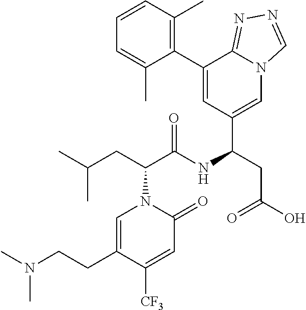 [SMILES: Cc1cccc(C)c1-c1cc([C@@H](CC(=O)O)NC(=O)[C@@H](CC(C)C)n2cc(CCN(C)C)c(C(F)(F)F)cc2=O)cn2cnnc12] | 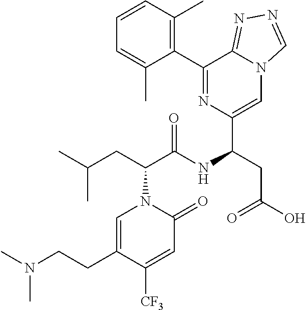 [SMILES: Cc1cccc(C)c1-c1nc([C@@H](CC(=O)O)NC(=O)[C@@H](CC(C)C)n2cc(CCN(C)C)c(C(F)(F)F)cc2=O)cn2cnnc12] |
| 75 | 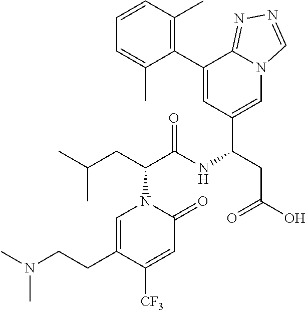 [SMILES: Cc1cccc(C)c1-c1cc([C@H](CC(=O)O)NC(=O)[C@@H](CC(C)C)n2cc(CCN(C)C)c(C(F)(F)F)cc2=O)cn2cnnc12] | 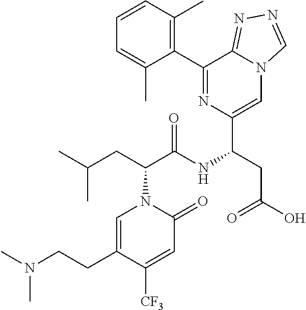 [SMILES: Cc1cccc(C)c1-c1nc([C@H](CC(=O)O)NC(=O)[C@@H](CC(C)C)n2cc(CCN(C)C)c(C(F)(F)F)cc2=O)cn2cnnc12] |
| 76 | 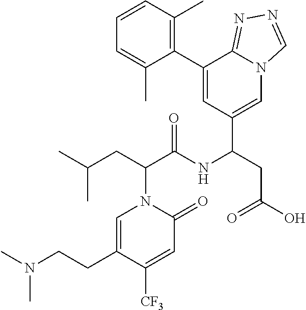 [SMILES: Cc1cccc(C)c1-c1cc(C(CC(=O)O)NC(=O)C(CC(C)C)n2cc(CCN(C)C)c(C(F)(F)F)cc2=O)cn2cnnc12] | 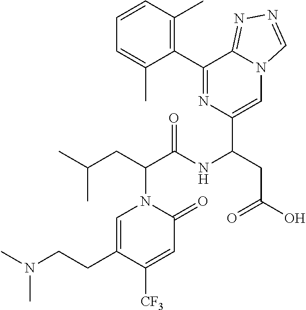 [SMILES: Cc1cccc(C)c1-c1nc(C(CC(=O)O)NC(=O)C(CC(C)C)n2cc(CCN(C)C)c(C(F)(F)F)cc2=O)cn2cnnc12] |
| 77 | 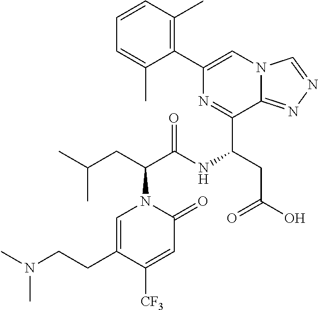 [SMILES: Cc1cccc(C)c1-c1cn2cnnc2c([C@H](CC(=O)O)NC(=O)[C@H](CC(C)C)n2cc(CCN(C)C)c(C(F)(F)F)cc2=O)n1] | 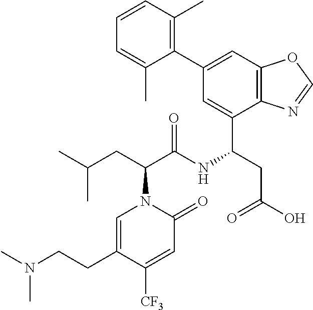 [SMILES: Cc1cccc(C)c1-c1cc([C@H](CC(=O)O)NC(=O)[C@H](CC(C)C)n2cc(CCN(C)C)c(C(F)(F)F)cc2=O)c2ncoc2c1] |
| 78 | 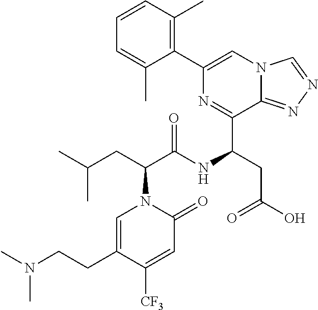 [SMILES: Cc1cccc(C)c1-c1cn2cnnc2c([C@@H](CC(=O)O)NC(=O)[C@H](CC(C)C)n2cc(CCN(C)C)c(C(F)(F)F)cc2=O)n1] | 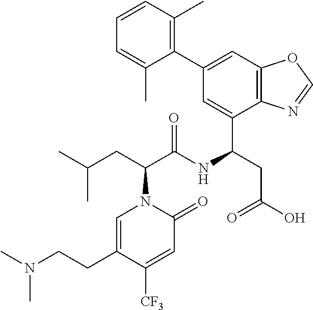 [SMILES: Cc1cccc(C)c1-c1cc([C@@H](CC(=O)O)NC(=O)[C@H](CC(C)C)n2cc(CCN(C)C)c(C(F)(F)F)cc2=O)c2ncoc2c1] |
| 79 | 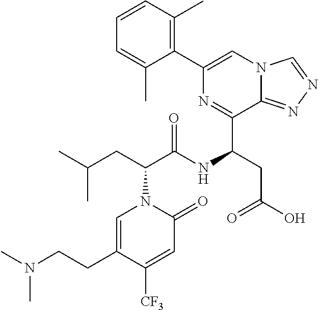 [SMILES: Cc1cccc(C)c1-c1cn2cnnc2c([C@@H](CC(=O)O)NC(=O)[C@@H](CC(C)C)n2cc(CCN(C)C)c(C(F)(F)F)cc2=O)n1] | 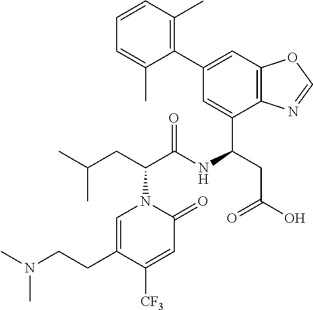 [SMILES: Cc1cccc(C)c1-c1cc([C@@H](CC(=O)O)NC(=O)[C@@H](CC(C)C)n2cc(CCN(C)C)c(C(F)(F)F)cc2=O)c2ncoc2c1] |
| 80 | 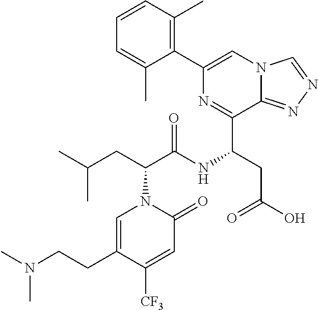 [SMILES: Cc1cccc(C)c1-c1cn2cnnc2c([C@H](CC(=O)O)NC(=O)[C@@H](CC(C)C)n2cc(CCN(C)C)c(C(F)(F)F)cc2=O)n1] | 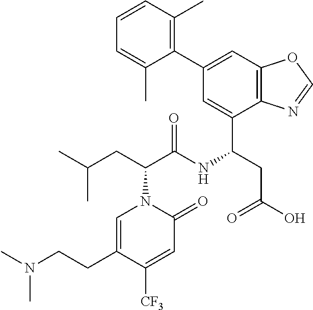 [SMILES: Cc1cccc(C)c1-c1cc([C@H](CC(=O)O)NC(=O)[C@@H](CC(C)C)n2cc(CCN(C)C)c(C(F)(F)F)cc2=O)c2ncoc2c1] |
| 81 | 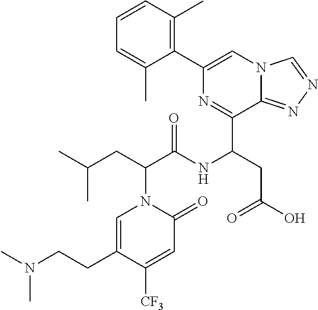 [SMILES: Cc1cccc(C)c1-c1cn2cnnc2c(C(CC(=O)O)NC(=O)C(CC(C)C)n2cc(CCN(C)C)c(C(F)(F)F)cc2=O)n1] | 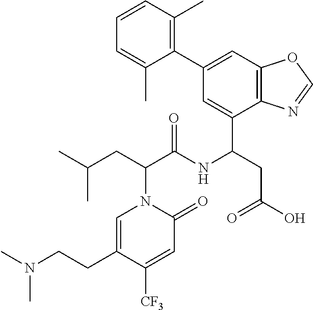 [SMILES: Cc1cccc(C)c1-c1cc(C(CC(=O)O)NC(=O)C(CC(C)C)n2cc(CCN(C)C)c(C(F)(F)F)cc2=O)c2ncoc2c1] |
| 82 | 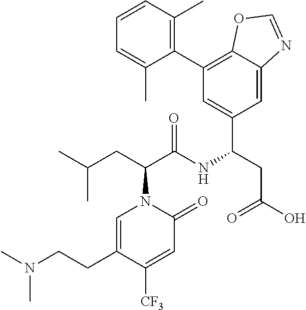 [SMILES: Cc1cccc(C)c1-c1cc([C@H](CC(=O)O)NC(=O)[C@H](CC(C)C)n2cc(CCN(C)C)c(C(F)(F)F)cc2=O)cc2ncoc12] | 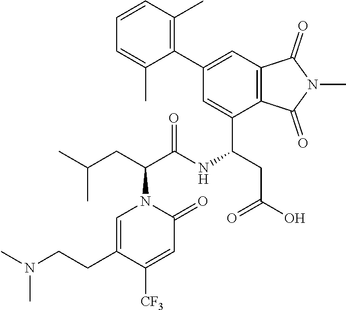 |
| 83 | 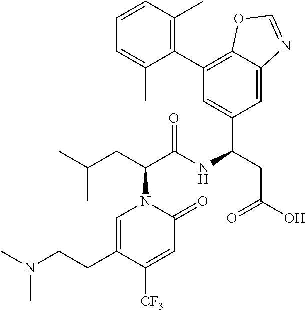 [SMILES: Cc1cccc(C)c1-c1cc([C@@H](CC(=O)O)NC(=O)[C@H](CC(C)C)n2cc(CCN(C)C)c(C(F)(F)F)cc2=O)cc2ncoc12] | 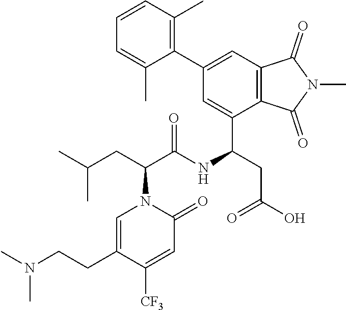 |
| 84 | 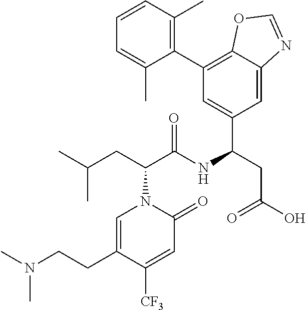 [SMILES: Cc1cccc(C)c1-c1cc([C@@H](CC(=O)O)NC(=O)[C@@H](CC(C)C)n2cc(CCN(C)C)c(C(F)(F)F)cc2=O)cc2ncoc12] | 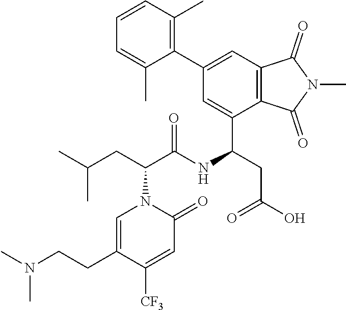 |
| 85 | 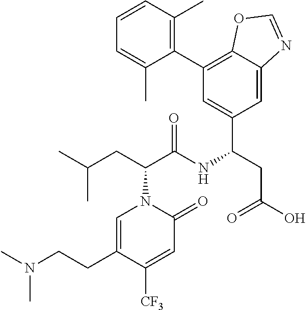 [SMILES: Cc1cccc(C)c1-c1cc([C@H](CC(=O)O)NC(=O)[C@@H](CC(C)C)n2cc(CCN(C)C)c(C(F)(F)F)cc2=O)cc2ncoc12] | 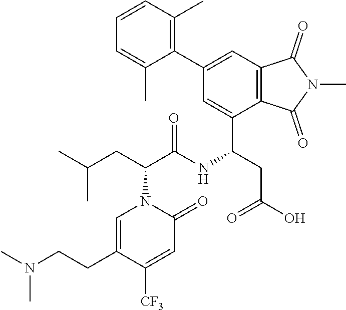 |
| 86 | 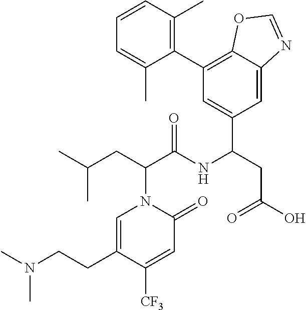 [SMILES: Cc1cccc(C)c1-c1cc(C(CC(=O)O)NC(=O)C(CC(C)C)n2cc(CCN(C)C)c(C(F)(F)F)cc2=O)cc2ncoc12] | 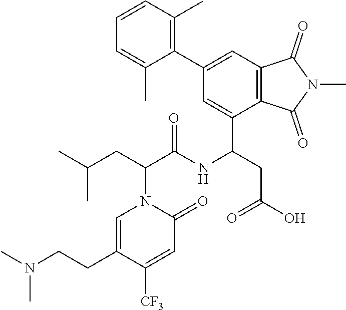 |
| 87 | 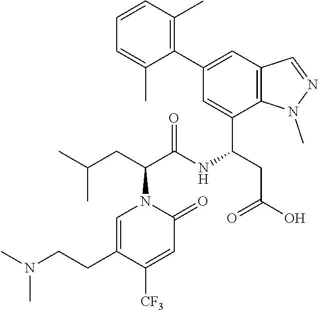 [SMILES: Cc1cccc(C)c1-c1cc([C@H](CC(=O)O)NC(=O)[C@H](CC(C)C)n2cc(CCN(C)C)c(C(F)(F)F)cc2=O)c2c(cnn2C)c1] | 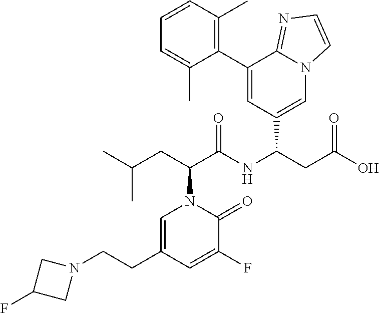 [SMILES: Cc1cccc(C)c1-c1cc([C@H](CC(=O)O)NC(=O)[C@H](CC(C)C)n2cc(CCN3CC(F)C3)cc(F)c2=O)cn2ccnc12] |
| 88 | 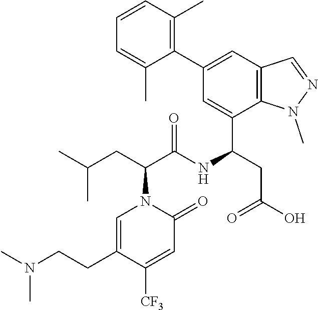 [SMILES: Cc1cccc(C)c1-c1cc([C@@H](CC(=O)O)NC(=O)[C@H](CC(C)C)n2cc(CCN(C)C)c(C(F)(F)F)cc2=O)c2c(cnn2C)c1] | 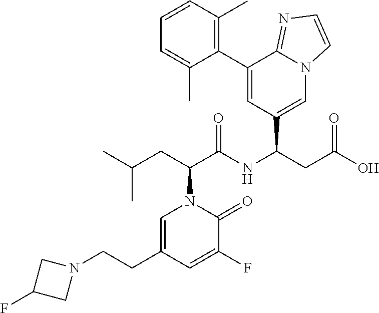 [SMILES: Cc1cccc(C)c1-c1cc([C@@H](CC(=O)O)NC(=O)[C@H](CC(C)C)n2cc(CCN3CC(F)C3)cc(F)c2=O)cn2ccnc12] |
| 89 | 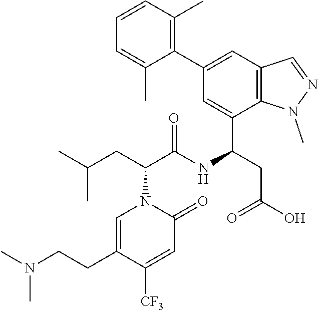 [SMILES: Cc1cccc(C)c1-c1cc([C@@H](CC(=O)O)NC(=O)[C@@H](CC(C)C)n2cc(CCN(C)C)c(C(F)(F)F)cc2=O)c2c(cnn2C)c1] | 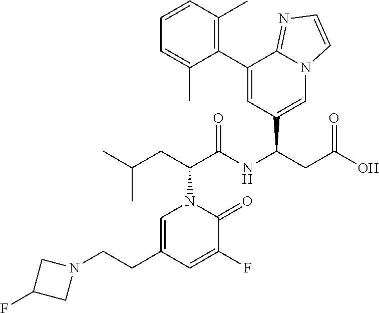 [SMILES: Cc1cccc(C)c1-c1cc([C@@H](CC(=O)O)NC(=O)[C@@H](CC(C)C)n2cc(CCN3CC(F)C3)cc(F)c2=O)cn2ccnc12] |
| 90 | 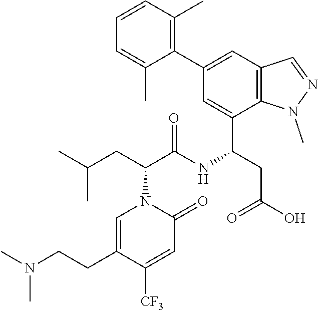 [SMILES: Cc1cccc(C)c1-c1cc([C@H](CC(=O)O)NC(=O)[C@@H](CC(C)C)n2cc(CCN(C)C)c(C(F)(F)F)cc2=O)c2c(cnn2C)c1] | 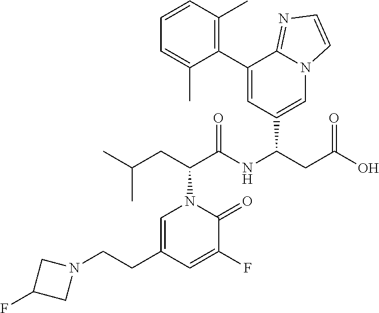 [SMILES: Cc1cccc(C)c1-c1cc([C@H](CC(=O)O)NC(=O)[C@@H](CC(C)C)n2cc(CCN3CC(F)C3)cc(F)c2=O)cn2ccnc12] |
| 91 | 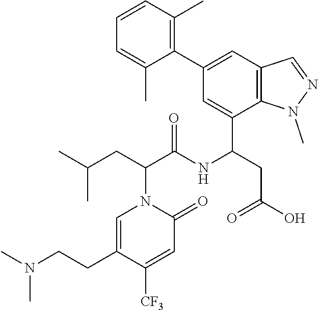 [SMILES: Cc1cccc(C)c1-c1cc(C(CC(=O)O)NC(=O)C(CC(C)C)n2cc(CCN(C)C)c(C(F)(F)F)cc2=O)c2c(cnn2C)c1] | 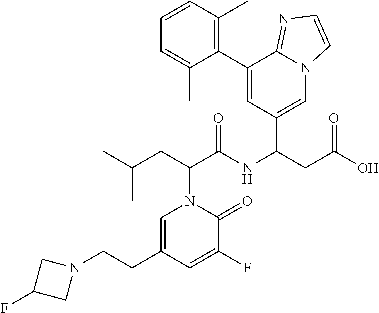 [SMILES: Cc1cccc(C)c1-c1cc(C(CC(=O)O)NC(=O)C(CC(C)C)n2cc(CCN3CC(F)C3)cc(F)c2=O)cn2ccnc12] |
| 92 | 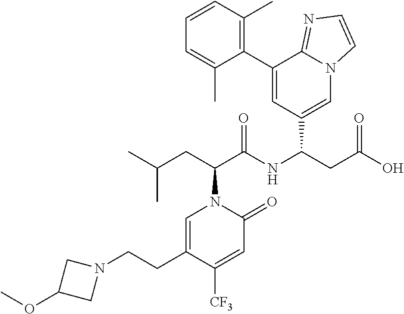 [SMILES: COC1CN(CCc2cn([C@@H](CC(C)C)C(=O)N[C@@H](CC(=O)O)c3cc(-c4c(C)cccc4C)c4nccn4c3)c(=O)cc2C(F)(F)F)C1] | 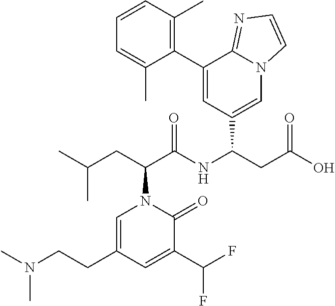 [SMILES: Cc1cccc(C)c1-c1cc([C@H](CC(=O)O)NC(=O)[C@H](CC(C)C)n2cc(CCN(C)C)cc(C(F)F)c2=O)cn2ccnc12] |
| 93 | 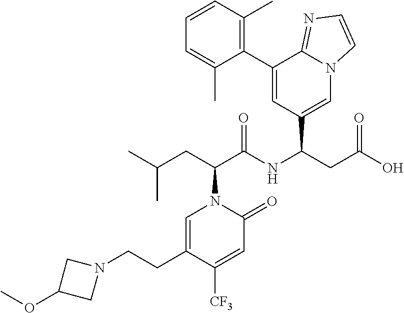 [SMILES: COC1CN(CCc2cn([C@@H](CC(C)C)C(=O)N[C@H](CC(=O)O)c3cc(-c4c(C)cccc4C)c4nccn4c3)c(=O)cc2C(F)(F)F)C1] | 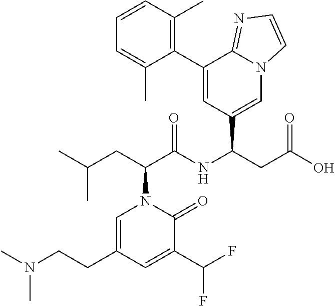 [SMILES: Cc1cccc(C)c1-c1cc([C@@H](CC(=O)O)NC(=O)[C@H](CC(C)C)n2cc(CCN(C)C)cc(C(F)F)c2=O)cn2ccnc12] |
| 94 | 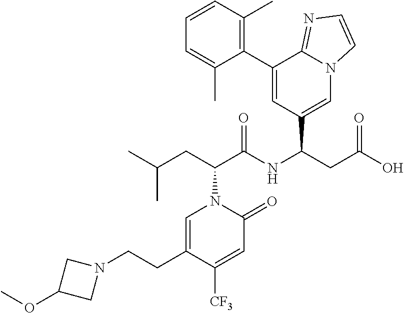 [SMILES: COC1CN(CCc2cn([C@H](CC(C)C)C(=O)N[C@H](CC(=O)O)c3cc(-c4c(C)cccc4C)c4nccn4c3)c(=O)cc2C(F)(F)F)C1] | 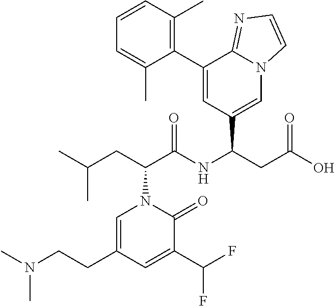 [SMILES: Cc1cccc(C)c1-c1cc([C@@H](CC(=O)O)NC(=O)[C@@H](CC(C)C)n2cc(CCN(C)C)cc(C(F)F)c2=O)cn2ccnc12] |
| 95 | 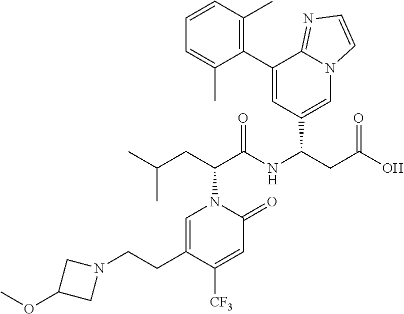 [SMILES: COC1CN(CCc2cn([C@H](CC(C)C)C(=O)N[C@@H](CC(=O)O)c3cc(-c4c(C)cccc4C)c4nccn4c3)c(=O)cc2C(F)(F)F)C1] | 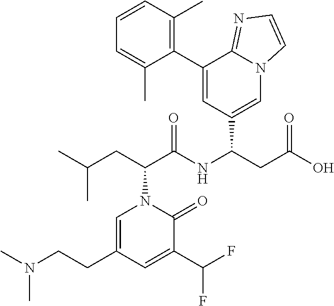 [SMILES: Cc1cccc(C)c1-c1cc([C@H](CC(=O)O)NC(=O)[C@@H](CC(C)C)n2cc(CCN(C)C)cc(C(F)F)c2=O)cn2ccnc12] |
| 96 | 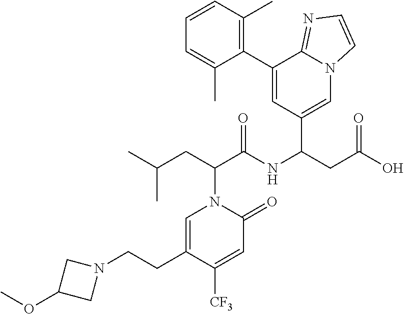 [SMILES: COC1CN(CCc2cn(C(CC(C)C)C(=O)NC(CC(=O)O)c3cc(-c4c(C)cccc4C)c4nccn4c3)c(=O)cc2C(F)(F)F)C1] | 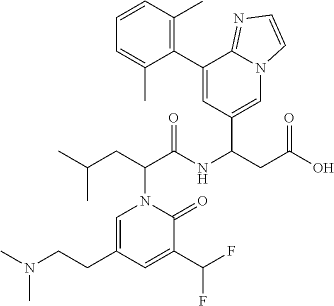 [SMILES: Cc1cccc(C)c1-c1cc(C(CC(=O)O)NC(=O)C(CC(C)C)n2cc(CCN(C)C)cc(C(F)F)c2=O)cn2ccnc12] |
| 97 | 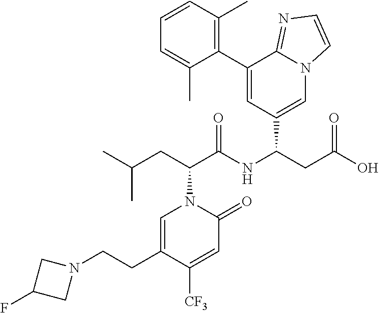 [SMILES: Cc1cccc(C)c1-c1cc([C@H](CC(=O)O)NC(=O)[C@@H](CC(C)C)n2cc(CCN3CC(F)C3)c(C(F)(F)F)cc2=O)cn2ccnc12] | 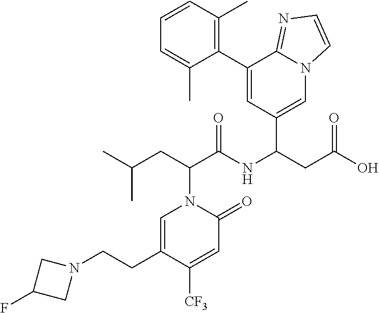 [SMILES: Cc1cccc(C)c1-c1cc(C(CC(=O)O)NC(=O)C(CC(C)C)n2cc(CCN3CC(F)C3)c(C(F)(F)F)cc2=O)cn2ccnc12] |
| 98 | 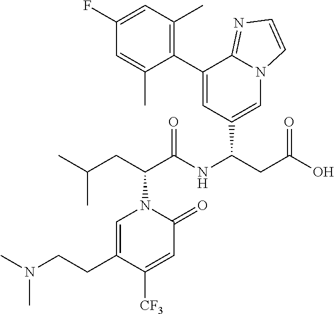 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cn2ccnc12] | 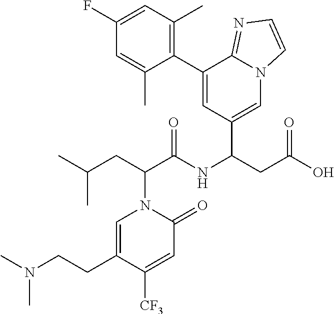 [SMILES: Cc1cc(F)cc(C)c1-c1cc(C(CC(=O)O)NC(=O)C(CC(C)C)n2cc(CCN(C)C)c(C(F)(F)F)cc2=O)cn2ccnc12] |
| 99 | 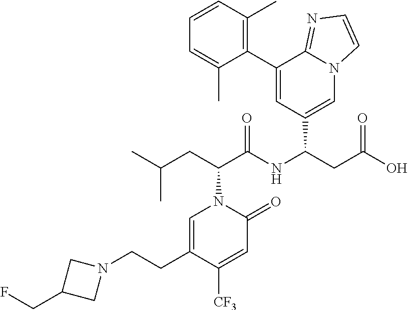 [SMILES: Cc1cccc(C)c1-c1cc([C@H](CC(=O)O)NC(=O)[C@@H](CC(C)C)n2cc(CCN3CC(CF)C3)c(C(F)(F)F)cc2=O)cn2ccnc12] | 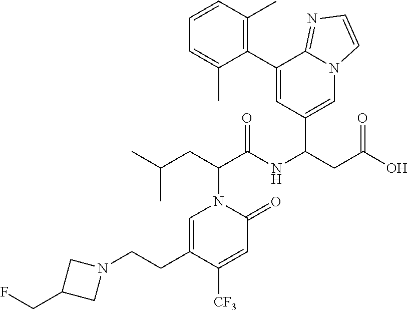 [SMILES: Cc1cccc(C)c1-c1cc(C(CC(=O)O)NC(=O)C(CC(C)C)n2cc(CCN3CC(CF)C3)c(C(F)(F)F)cc2=O)cn2ccnc12] |
| 100 | 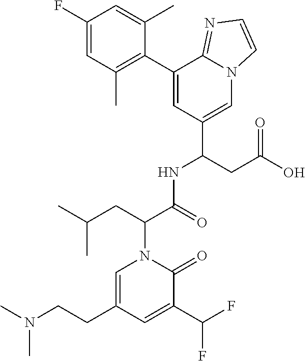 [SMILES: Cc1cc(F)cc(C)c1-c1cc(C(CC(=O)O)NC(=O)C(CC(C)C)n2cc(CCN(C)C)cc(C(F)F)c2=O)cn2ccnc12] | 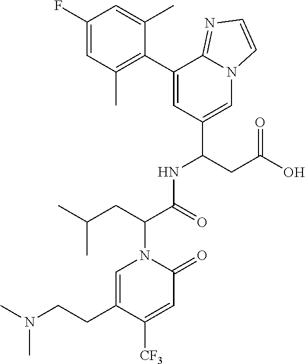 [SMILES: Cc1cc(F)cc(C)c1-c1cc(C(CC(=O)O)NC(=O)C(CC(C)C)n2cc(CCN(C)C)c(C(F)(F)F)cc2=O)cn2ccnc12] |
| 101 | 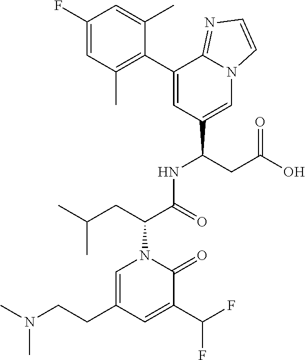 [SMILES: Cc1cc(F)cc(C)c1-c1cc([C@@H](CC(=O)O)NC(=O)[C@@H](CC(C)C)n2cc(CCN(C)C)cc(C(F)F)c2=O)cn2ccnc12] | 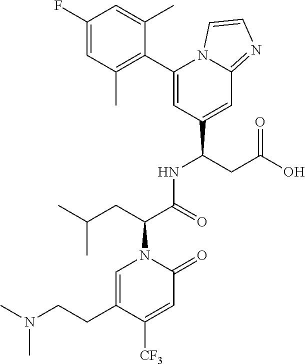 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)cc2nccn12] |
| 102 | 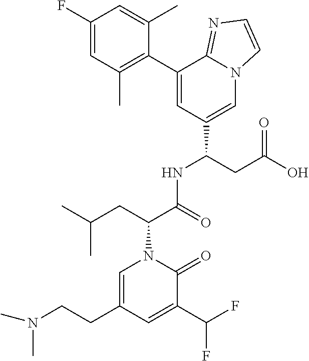 [SMILES: Cc1cc(F)cc(C)c1-c1cc([C@H](CC(=O)O)NC(=O)[C@@H](CC(C)C)n2cc(CCN(C)C)cc(C(F)F)c2=O)cn2ccnc12] | 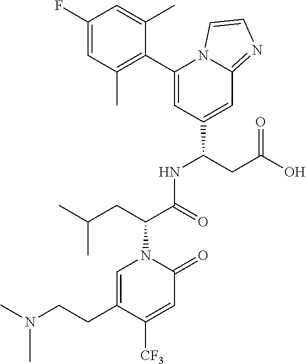 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cc2nccn12] |
| 103 | 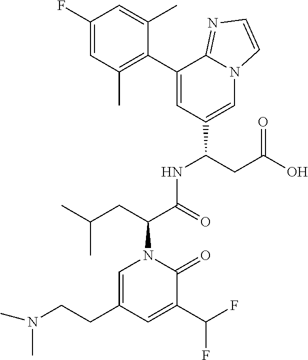 [SMILES: Cc1cc(F)cc(C)c1-c1cc([C@H](CC(=O)O)NC(=O)[C@H](CC(C)C)n2cc(CCN(C)C)cc(C(F)F)c2=O)cn2ccnc12] | 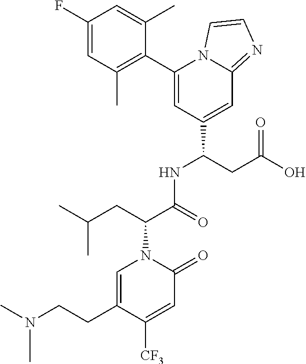 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cc2nccn12] |
| 104 | 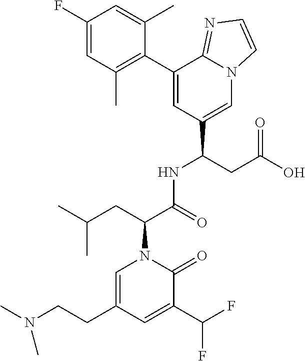 [SMILES: Cc1cc(F)cc(C)c1-c1cc([C@@H](CC(=O)O)NC(=O)[C@H](CC(C)C)n2cc(CCN(C)C)cc(C(F)F)c2=O)cn2ccnc12] | 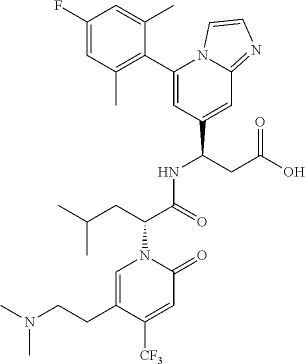 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)cc2nccn12] |
| 105 | 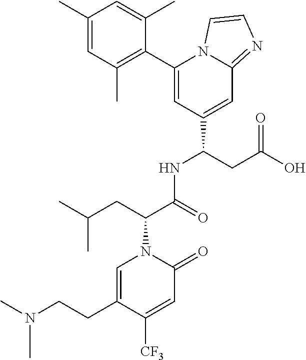 [SMILES: Cc1cc(C)c(-c2cc([C@H](CC(=O)O)NC(=O)[C@@H](CC(C)C)n3cc(CCN(C)C)c(C(F)(F)F)cc3=O)cc3nccn23)c(C)c1] | 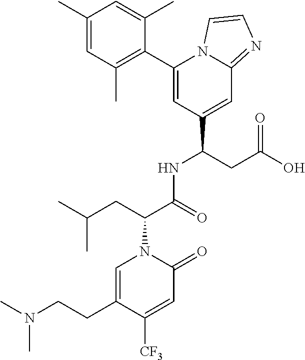 [SMILES: Cc1cc(C)c(-c2cc([C@@H](CC(=O)O)NC(=O)[C@@H](CC(C)C)n3cc(CCN(C)C)c(C(F)(F)F)cc3=O)cc3nccn23)c(C)c1] |
| 106 | 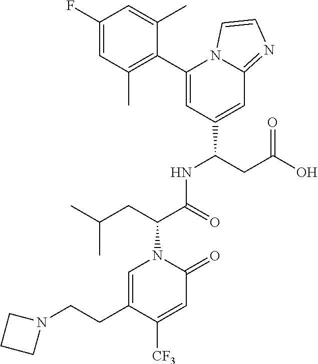 [SMILES: Cc1cc(F)cc(C)c1-c1cc([C@H](CC(=O)O)NC(=O)[C@@H](CC(C)C)n2cc(CCN3CCC3)c(C(F)(F)F)cc2=O)cc2nccn12] | 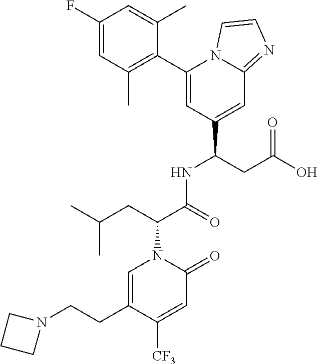 [SMILES: Cc1cc(F)cc(C)c1-c1cc([C@@H](CC(=O)O)NC(=O)[C@@H](CC(C)C)n2cc(CCN3CCC3)c(C(F)(F)F)cc2=O)cc2nccn12] |
| 107 | 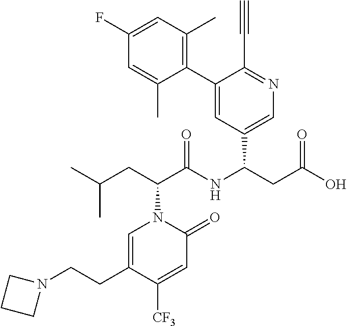 [SMILES: C#Cc1ncc([C@H](CC(=O)O)NC(=O)[C@@H](CC(C)C)n2cc(CCN3CCC3)c(C(F)(F)F)cc2=O)cc1-c1c(C)cc(F)cc1C] | 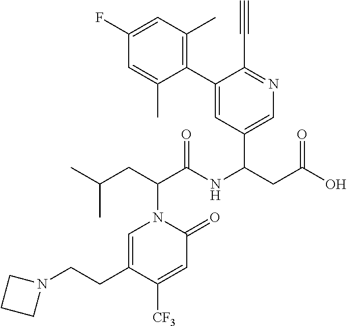 [SMILES: C#Cc1ncc(C(CC(=O)O)NC(=O)C(CC(C)C)n2cc(CCN3CCC3)c(C(F)(F)F)cc2=O)cc1-c1c(C)cc(F)cc1C] |
| 108 | 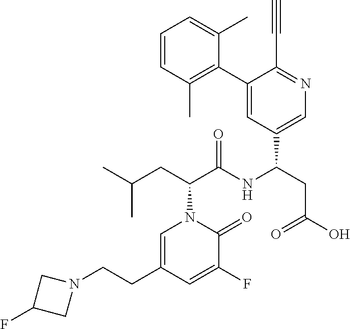 [SMILES: C#Cc1ncc([C@H](CC(=O)O)NC(=O)[C@@H](CC(C)C)n2cc(CCN3CC(F)C3)cc(F)c2=O)cc1-c1c(C)cccc1C] | 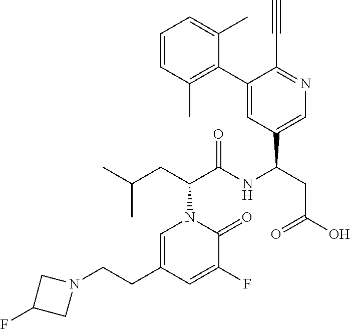 [SMILES: C#Cc1ncc([C@@H](CC(=O)O)NC(=O)[C@@H](CC(C)C)n2cc(CCN3CC(F)C3)cc(F)c2=O)cc1-c1c(C)cccc1C] |
| 109 | 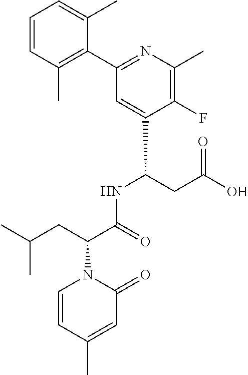 [SMILES: Cc1ccn([C@H](CC(C)C)C(=O)N[C@@H](CC(=O)O)c2cc(-c3c(C)cccc3C)nc(C)c2F)c(=O)c1] | 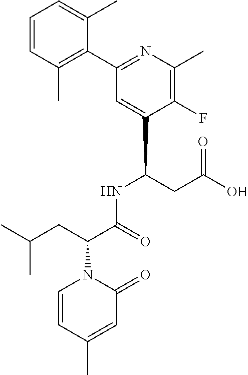 [SMILES: Cc1ccn([C@H](CC(C)C)C(=O)N[C@H](CC(=O)O)c2cc(-c3c(C)cccc3C)nc(C)c2F)c(=O)c1] |

TABLE E-2

| | | |
|---|---|---|
| 110 | 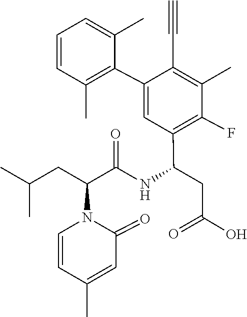 [SMILES: C#Cc1c(-c2c(C)cccc2C)cc([C@H](CC(=O)O)NC(=O)[C@H](CC(C)C)n2ccc(C)cc2=O)c(F)c1C] | 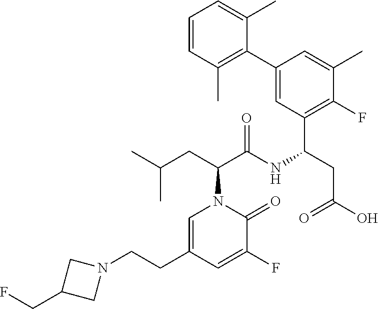 [SMILES: Cc1cc(-c2c(C)cccc2C)cc([C@H](CC(=O)O)NC(=O)[C@H](CC(C)C)n2cc(CCN3CC(CF)C3)cc(F)c2=O)c1F] |
| 111 | 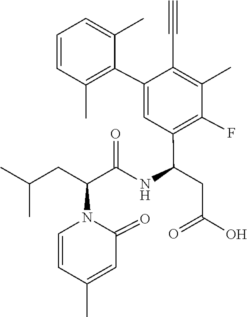 [SMILES: C#Cc1c(-c2c(C)cccc2C)cc([C@@H](CC(=O)O)NC(=O)[C@H](CC(C)C)n2ccc(C)cc2=O)c(F)c1C] | 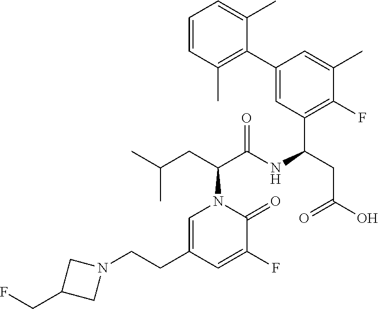 [SMILES: Cc1cc(-c2c(C)cccc2C)cc([C@@H](CC(=O)O)NC(=O)[C@H](CC(C)C)n2cc(CCN3CC(CF)C3)cc(F)c2=O)c1F] |
| 112 | 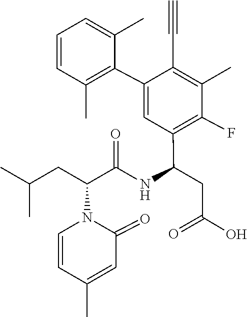 [SMILES: C#Cc1c(-c2c(C)cccc2C)cc([C@@H](CC(=O)O)NC(=O)[C@@H](CC(C)C)n2ccc(C)cc2=O)c(F)c1C] | 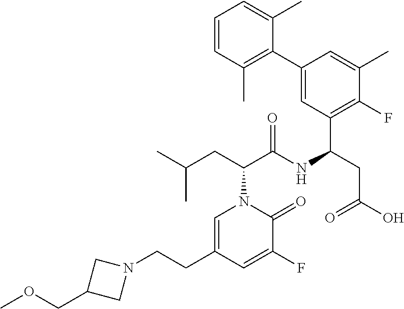 [SMILES: COCC1CN(CCc2cc(F)c(=O)n([C@H](CC(C)C)C(=O)N[C@H](CC(=O)O)c3cc(-c4c(C)cccc4C)cc(C)c3F)c2)C1] |
| 113 | 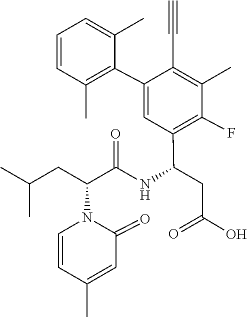 [SMILES: C#Cc1c(-c2c(C)cccc2C)cc([C@H](CC(=O)O)NC(=O)[C@@H](CC(C)C)n2ccc(C)cc2=O)c(F)c1C] | 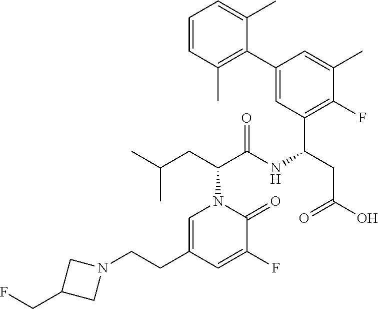 [SMILES: Cc1cc(-c2c(C)cccc2C)cc([C@H](CC(=O)O)NC(=O)[C@@H](CC(C)C)n2cc(CCN3CC(CF)C3)cc(F)c2=O)c1F] |
| 114 | 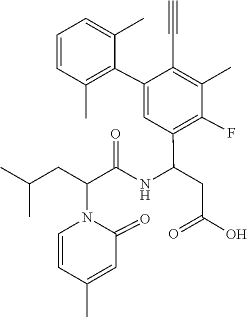 [SMILES: C#Cc1c(-c2c(C)cccc2C)cc(C(CC(=O)O)NC(=O)C(CC(C)C)n2ccc(C)cc2=O)c(F)c1C] | 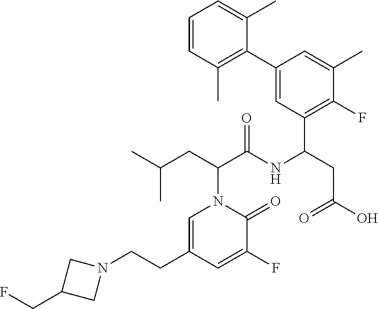 [SMILES: Cc1cc(-c2c(C)cccc2C)cc(C(CC(=O)O)NC(=O)C(CC(C)C)n2cc(CCN3CC(CF)C3)cc(F)c2=O)c1F] |
| 115 | 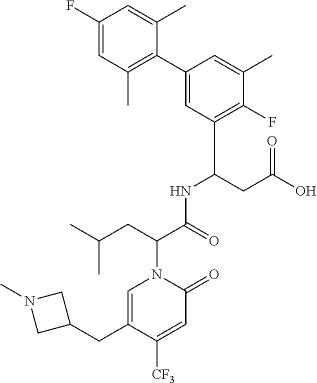 [SMILES: Cc1cc(-c2c(C)cc(F)cc2C)cc(C(CC(=O)O)NC(=O)C(CC(C)C)n2cc(CC3CN(C)C3)c(C(F)(F)F)cc2=O)c1F] | 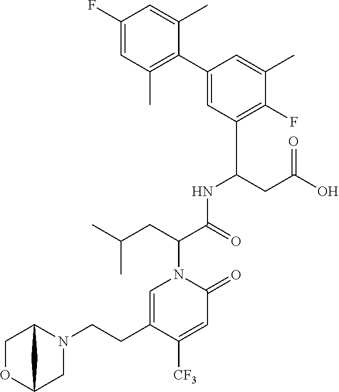 [SMILES: Cc1cc(-c2c(C)cc(F)cc2C)cc(C(CC(=O)O)NC(=O)C(CC(C)C)n2cc(CCN3C[C@H]4C[C@@H]3CO4)c(C(F)(F)F)cc2=O)c1F] |
| 116 | 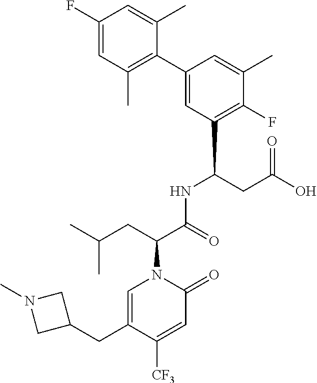 [SMILES: Cc1cc(-c2c(C)cc(F)cc2C)cc([C@@H](CC(=O)O)NC(=O)[C@H](CC(C)C)n2cc(CC3CN(C)C3)c(C(F)(F)F)cc2=O)c1F] | 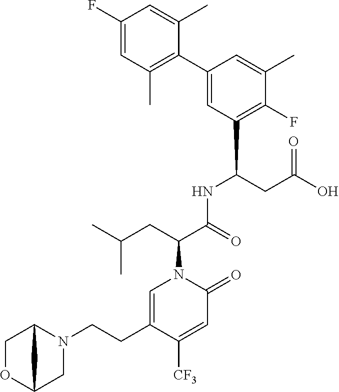 [SMILES: Cc1cc(-c2c(C)cc(F)cc2C)cc([C@@H](CC(=O)O)NC(=O)[C@H](CC(C)C)n2cc(CCN3C[C@H]4C[C@@H]3CO4)c(C(F)(F)F)cc2=O)c1F] |
| 117 | 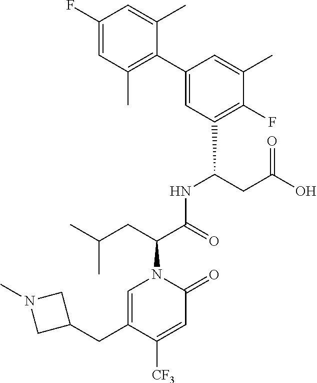 [SMILES: Cc1cc(-c2c(C)cc(F)cc2C)cc([C@H](CC(=O)O)NC(=O)[C@H](CC(C)C)n2cc(CC3CN(C)C3)c(C(F)(F)F)cc2=O)c1F] | 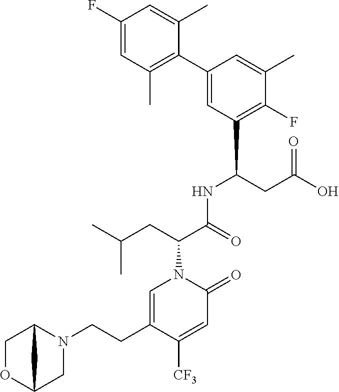 [SMILES: Cc1cc(-c2c(C)cc(F)cc2C)cc([C@@H](CC(=O)O)NC(=O)[C@@H](CC(C)C)n2cc(CCN3C[C@H]4C[C@@H]3CO4)c(C(F)(F)F)cc2=O)c1F] |
| 118 | 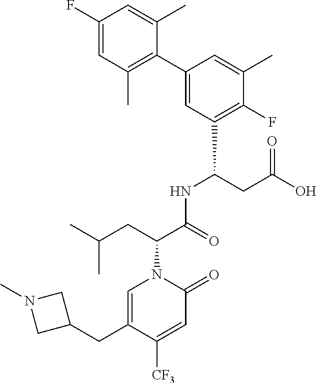 [SMILES: Cc1cc(-c2c(C)cc(F)cc2C)cc([C@H](CC(=O)O)NC(=O)[C@@H](CC(C)C)n2cc(CC3CN(C)C3)c(C(F)(F)F)cc2=O)c1F] | 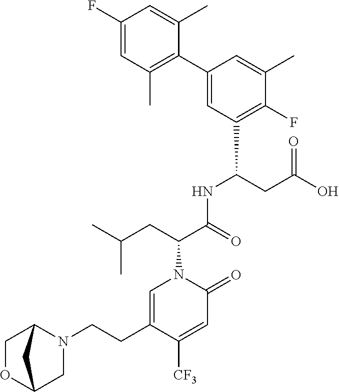 [SMILES: Cc1cc(-c2c(C)cc(F)cc2C)cc([C@H](CC(=O)O)NC(=O)[C@@H](CC(C)C)n2cc(CCN3C[C@H]4C[C@@H]3CO4)c(C(F)(F)F)cc2=O)c1F] |
| 119 | 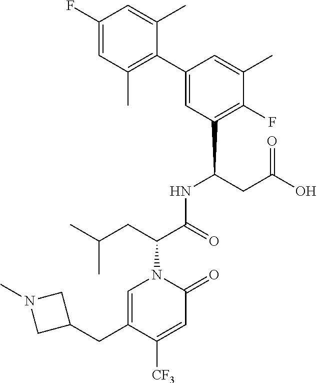 [SMILES: Cc1cc(-c2c(C)cc(F)cc2C)cc([C@@H](CC(=O)O)NC(=O)[C@@H](CC(C)C)n2cc(CC3CN(C)C3)c(C(F)(F)F)cc2=O)c1F] | 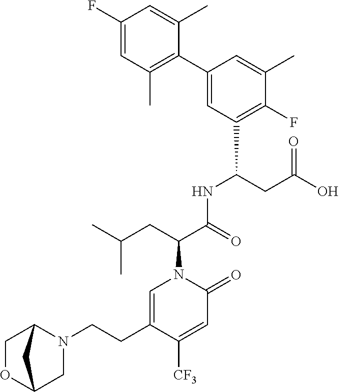 [SMILES: Cc1cc(-c2c(C)cc(F)cc2C)cc([C@H](CC(=O)O)NC(=O)[C@H](CC(C)C)n2cc(CCN3C[C@H]4C[C@@H]3CO4)c(C(F)(F)F)cc2=O)c1F] |
| 120 | 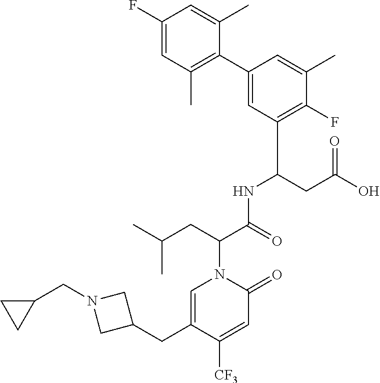 [SMILES: Cc1cc(-c2c(C)cc(F)cc2C)cc(C(CC(=O)O)NC(=O)C(CC(C)C)n2cc(CC3CN(CC4CC4)C3)c(C(F)(F)F)cc2=O)c1F] | 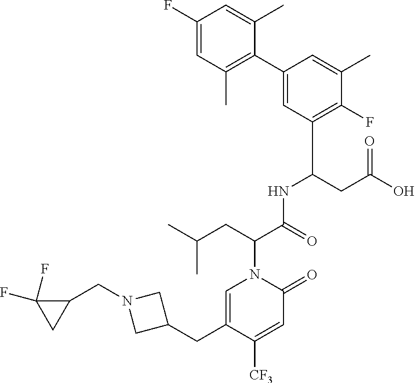 [SMILES: Cc1cc(-c2c(C)cc(F)cc2C)cc(C(CC(=O)O)NC(=O)C(CC(C)C)n2cc(CC3CN(CC4CC4(F)F)C3)c(C(F)(F)F)cc2=O)c1F] |
| 121 | 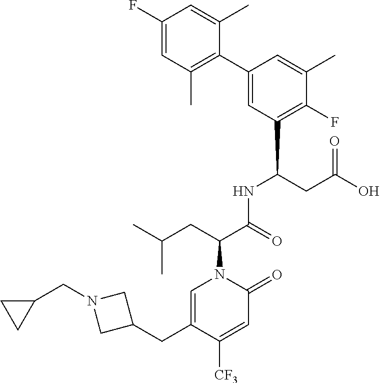 [SMILES: Cc1cc(-c2c(C)cc(F)cc2C)cc([C@@H](CC(=O)O)NC(=O)[C@H](CC(C)C)n2cc(CC3CN(CC4CC4)C3)c(C(F)(F)F)cc2=O)c1F] | 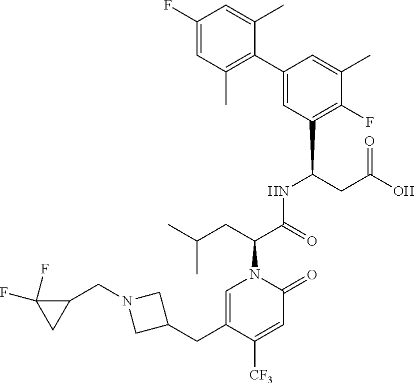 [SMILES: Cc1cc(-c2c(C)cc(F)cc2C)cc([C@@H](CC(=O)O)NC(=O)[C@H](CC(C)C)n2cc(CC3CN(CC4CC4(F)F)C3)c(C(F)(F)F)cc2=O)c1F] |
| 122 | 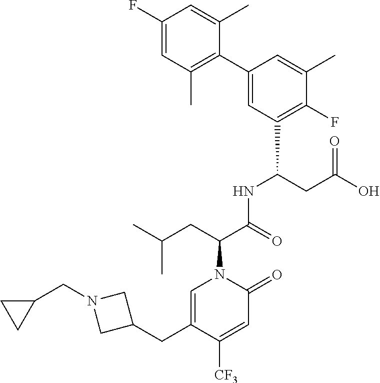 [SMILES: Cc1cc(-c2c(C)cc(F)cc2C)cc([C@H](CC(=O)O)NC(=O)[C@H](CC(C)C)n2cc(CC3CN(CC4CC4)C3)c(C(F)(F)F)cc2=O)c1F] | 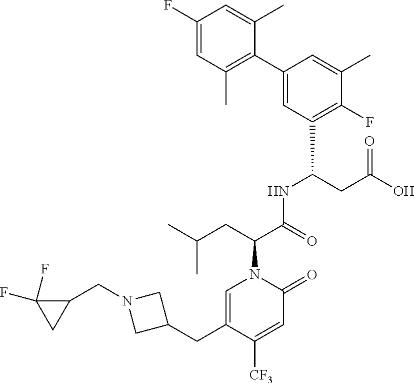 [SMILES: Cc1cc(-c2c(C)cc(F)cc2C)cc([C@H](CC(=O)O)NC(=O)[C@H](CC(C)C)n2cc(CC3CN(CC4CC4(F)F)C3)c(C(F)(F)F)cc2=O)c1F] |
| 123 | 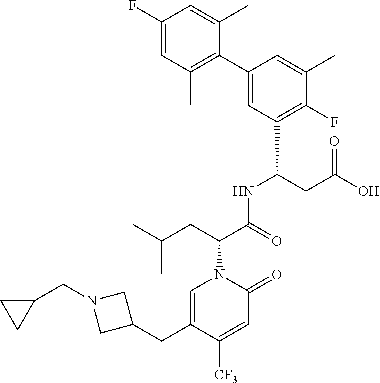 [SMILES: Cc1cc(-c2c(C)cc(F)cc2C)cc([C@H](CC(=O)O)NC(=O)[C@@H](CC(C)C)n2cc(CC3CN(CC4CC4)C3)c(C(F)(F)F)cc2=O)c1F] | 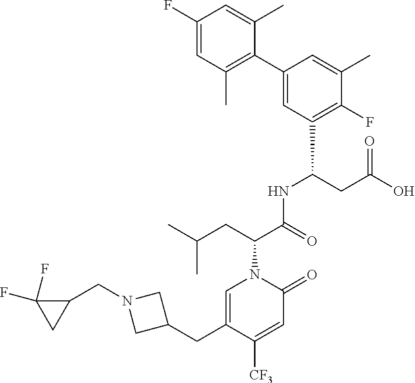 [SMILES: Cc1cc(-c2c(C)cc(F)cc2C)cc([C@H](CC(=O)O)NC(=O)[C@@H](CC(C)C)n2cc(CC3CN(CC4CC4(F)F)C3)c(C(F)(F)F)cc2=O)c1F] |
| 124 | 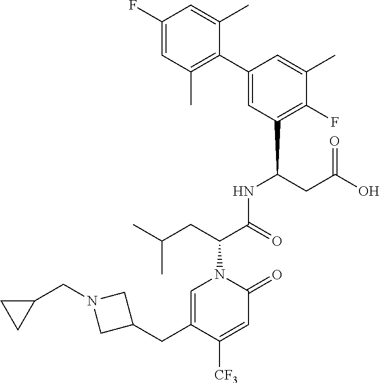 [SMILES: Cc1cc(-c2c(C)cc(F)cc2C)cc([C@@H](CC(=O)O)NC(=O)[C@@H](CC(C)C)n2cc(CC3CN(CC4CC4)C3)c(C(F)(F)F)cc2=O)c1F] | 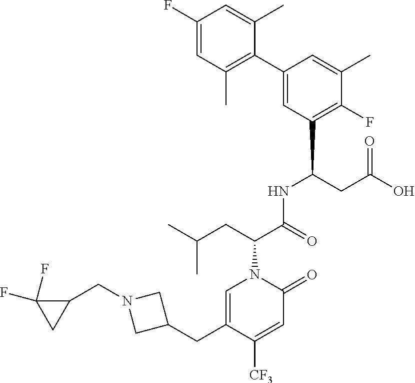 [SMILES: Cc1cc(-c2c(C)cc(F)cc2C)cc([C@@H](CC(=O)O)NC(=O)[C@@H](CC(C)C)n2cc(CC3CN(CC4CC4(F)F)C3)c(C(F)(F)F)cc2=O)c1F] |
| 125 | 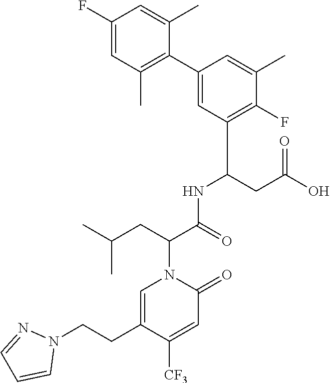 [SMILES: Cc1cc(-c2c(C)cc(F)cc2C)cc(C(CC(=O)O)NC(=O)C(CC(C)C)n2cc(CCn3cccn3)c(C(F)(F)F)cc2=O)c1F] | 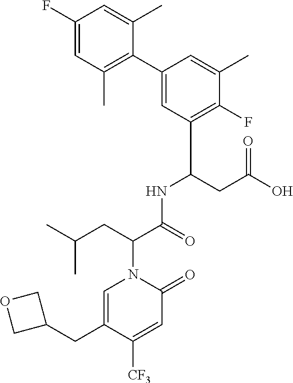 [SMILES: Cc1cc(-c2c(C)cc(F)cc2C)cc(C(CC(=O)O)NC(=O)C(CC(C)C)n2cc(CC3COC3)c(C(F)(F)F)cc2=O)c1F] |
| 126 | 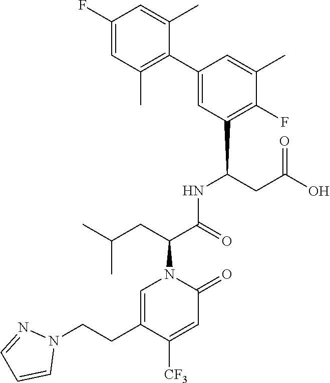 [SMILES: Cc1cc(-c2c(C)cc(F)cc2C)cc([C@@H](CC(=O)O)NC(=O)[C@H](CC(C)C)n2cc(CCn3cccn3)c(C(F)(F)F)cc2=O)c1F] | 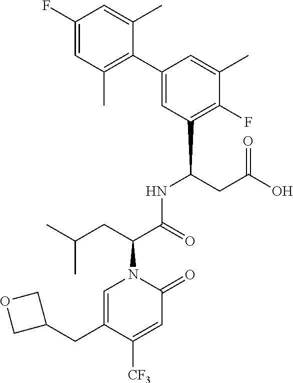 [SMILES: Cc1cc(-c2c(C)cc(F)cc2C)cc([C@@H](CC(=O)O)NC(=O)[C@H](CC(C)C)n2cc(CC3COC3)c(C(F)(F)F)cc2=O)c1F] |
| 127 | 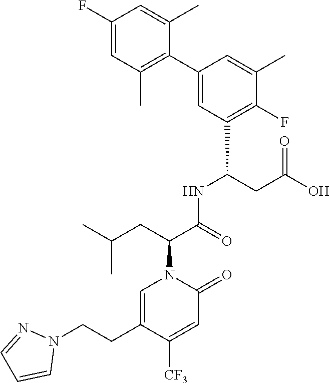 [SMILES: Cc1cc(-c2c(C)cc(F)cc2C)cc([C@H](CC(=O)O)NC(=O)[C@H](CC(C)C)n2cc(CCn3cccn3)c(C(F)(F)F)cc2=O)c1F] | 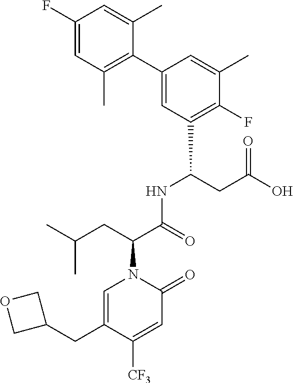 [SMILES: Cc1cc(-c2c(C)cc(F)cc2C)cc([C@H](CC(=O)O)NC(=O)[C@H](CC(C)C)n2cc(CC3COC3)c(C(F)(F)F)cc2=O)c1F] |
| 128 | 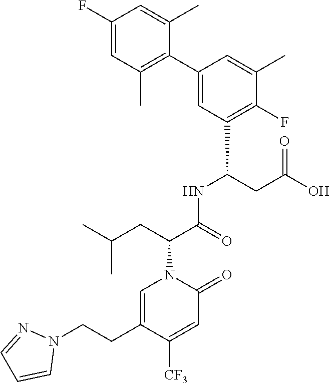 [SMILES: Cc1cc(-c2c(C)cc(F)cc2C)cc([C@H](CC(=O)O)NC(=O)[C@@H](CC(C)C)n2cc(CCn3cccn3)c(C(F)(F)F)cc2=O)c1F] | 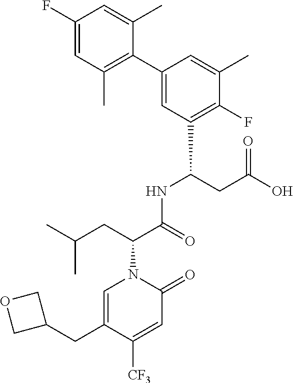 [SMILES: Cc1cc(-c2c(C)cc(F)cc2C)cc([C@H](CC(=O)O)NC(=O)[C@@H](CC(C)C)n2cc(CC3COC3)c(C(F)(F)F)cc2=O)c1F] |
| 129 | 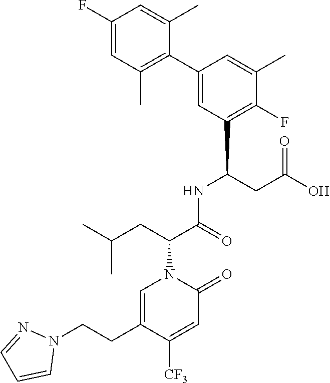 [SMILES: Cc1cc(-c2c(C)cc(F)cc2C)cc([C@@H](CC(=O)O)NC(=O)[C@@H](CC(C)C)n2cc(CCn3cccn3)c(C(F)(F)F)cc2=O)c1F] | 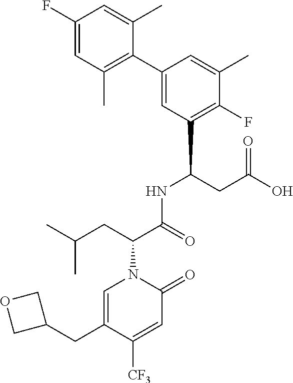 [SMILES: Cc1cc(-c2c(C)cc(F)cc2C)cc([C@@H](CC(=O)O)NC(=O)[C@@H](CC(C)C)n2cc(CC3COC3)c(C(F)(F)F)cc2=O)c1F] |
| 130 | 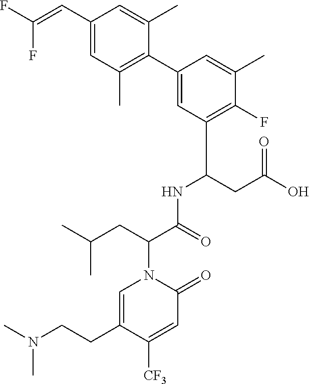 [SMILES: Cc1cc(-c2c(C)cc(C=C(F)F)cc2C)cc(C(CC(=O)O)NC(=O)C(CC(C)C)n2cc(CCN(C)C)c(C(F)(F)F)cc2=O)c1F] | 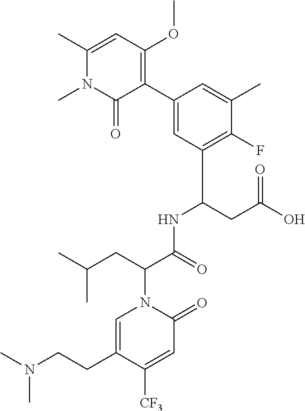 [SMILES: COc1cc(C)n(C)c(=O)c1-c1cc(C)c(F)c(C(CC(=O)O)NC(=O)C(CC(C)C)n2cc(CCN(C)C)c(C(F)(F)F)cc2=O)c1] |
| 131 | 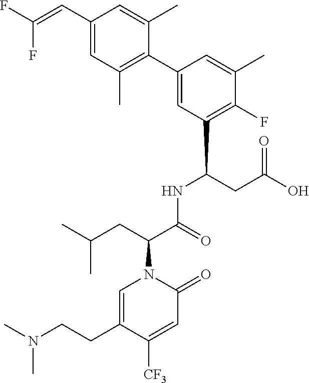 [SMILES: Cc1cc(-c2c(C)cc(C=C(F)F)cc2C)cc([C@@H](CC(=O)O)NC(=O)[C@H](CC(C)C)n2cc(CCN(C)C)c(C(F)(F)F)cc2=O)c1F] | 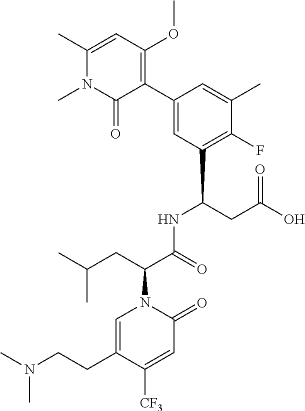 [SMILES: COc1cc(C)n(C)c(=O)c1-c1cc(C)c(F)c([C@@H](CC(=O)O)NC(=O)[C@H](CC(C)C)n2cc(CCN(C)C)c(C(F)(F)F)cc2=O)c1] |
| 132 | 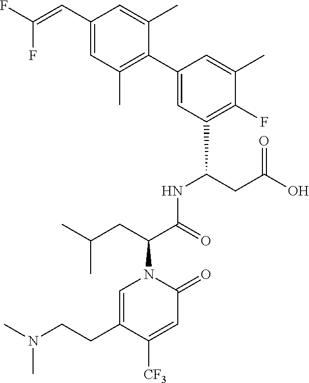 [SMILES: Cc1cc(-c2c(C)cc(C=C(F)F)cc2C)cc([C@H](CC(=O)O)NC(=O)[C@H](CC(C)C)n2cc(CCN(C)C)c(C(F)(F)F)cc2=O)c1F] | 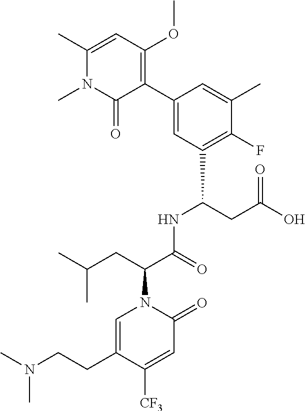 [SMILES: COc1cc(C)n(C)c(=O)c1-c1cc(C)c(F)c([C@H](CC(=O)O)NC(=O)[C@H](CC(C)C)n2cc(CCN(C)C)c(C(F)(F)F)cc2=O)c1] |
| 133 | 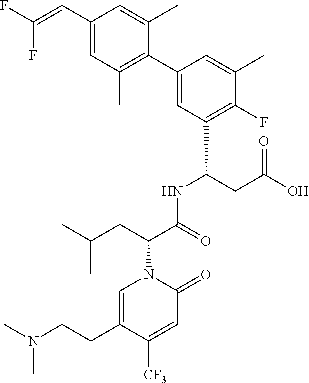 [SMILES: Cc1cc(-c2c(C)cc(C=C(F)F)cc2C)cc([C@H](CC(=O)O)NC(=O)[C@@H](CC(C)C)n2cc(CCN(C)C)c(C(F)(F)F)cc2=O)c1F] | 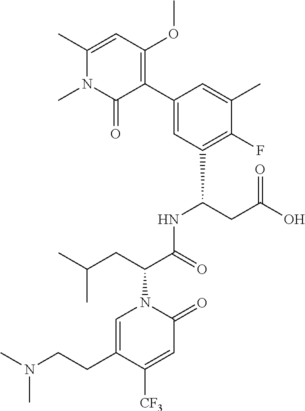 [SMILES: COc1cc(C)n(C)c(=O)c1-c1cc(C)c(F)c([C@H](CC(=O)O)NC(=O)[C@@H](CC(C)C)n2cc(CCN(C)C)c(C(F)(F)F)cc2=O)c1] |
| 134 | 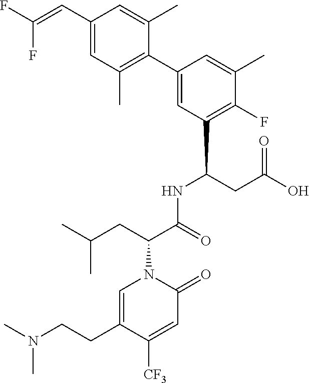 [SMILES: Cc1cc(-c2c(C)cc(C=C(F)F)cc2C)cc([C@@H](CC(=O)O)NC(=O)[C@@H](CC(C)C)n2cc(CCN(C)C)c(C(F)(F)F)cc2=O)c1F] | 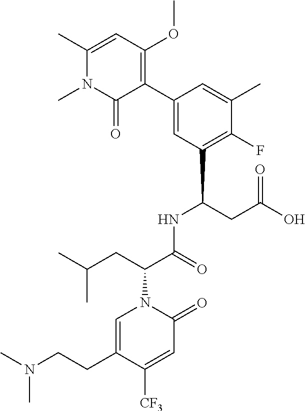 [SMILES: COc1cc(C)n(C)c(=O)c1-c1cc(C)c(F)c([C@@H](CC(=O)O)NC(=O)[C@@H](CC(C)C)n2cc(CCN(C)C)c(C(F)(F)F)cc2=O)c1] |
| 135 | 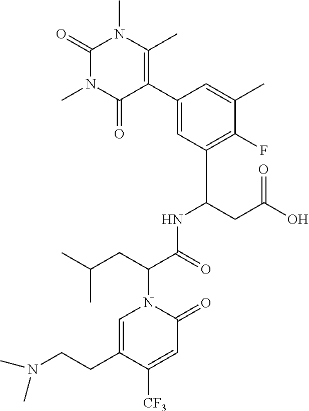 [SMILES: Cc1cc(-c2c(C)n(C)c(=O)n(C)c2=O)cc(C(CC(=O)O)NC(=O)C(CC(C)C)n2cc(CCN(C)C)c(C(F)(F)F)cc2=O)c1F] | 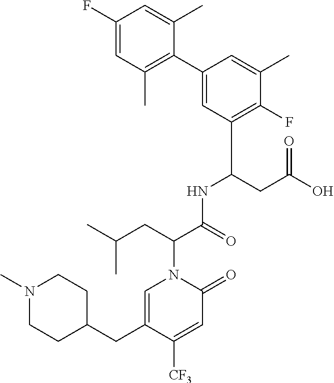 [SMILES: Cc1cc(-c2c(C)cc(F)cc2C)cc(C(CC(=O)O)NC(=O)C(CC(C)C)n2cc(CC3CCN(C)CC3)c(C(F)(F)F)cc2=O)c1F] |
| 136 | 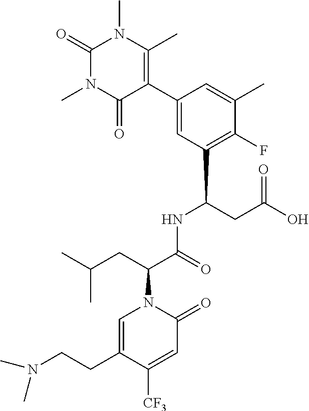 [SMILES: Cc1cc(-c2c(C)n(C)c(=O)n(C)c2=O)cc([C@@H](CC(=O)O)NC(=O)[C@H](CC(C)C)n2cc(CCN(C)C)c(C(F)(F)F)cc2=O)c1F] | 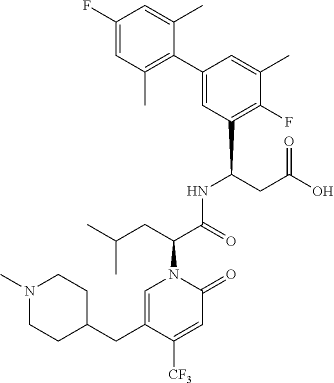 [SMILES: Cc1cc(-c2c(C)cc(F)cc2C)cc([C@@H](CC(=O)O)NC(=O)[C@H](CC(C)C)n2cc(CC3CCN(C)CC3)c(C(F)(F)F)cc2=O)c1F] |
| 137 | 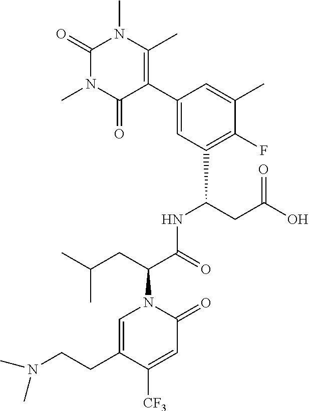 [SMILES: Cc1cc(-c2c(C)n(C)c(=O)n(C)c2=O)cc([C@H](CC(=O)O)NC(=O)[C@H](CC(C)C)n2cc(CCN(C)C)c(C(F)(F)F)cc2=O)c1F] | 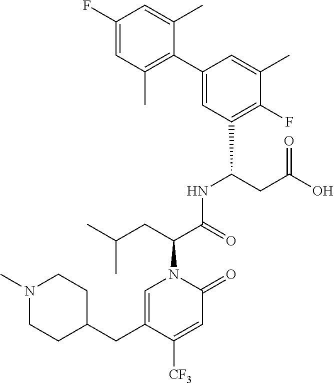 [SMILES: Cc1cc(-c2c(C)cc(F)cc2C)cc([C@H](CC(=O)O)NC(=O)[C@H](CC(C)C)n2cc(CC3CCN(C)CC3)c(C(F)(F)F)cc2=O)c1F] |
| 138 | 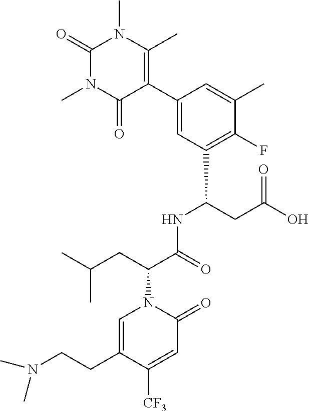 [SMILES: Cc1cc(-c2c(C)n(C)c(=O)n(C)c2=O)cc([C@H](CC(=O)O)NC(=O)[C@@H](CC(C)C)n2cc(CCN(C)C)c(C(F)(F)F)cc2=O)c1F] | 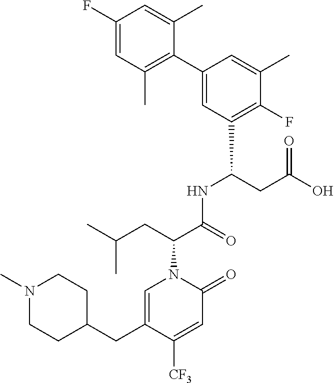 [SMILES: Cc1cc(-c2c(C)cc(F)cc2C)cc([C@H](CC(=O)O)NC(=O)[C@@H](CC(C)C)n2cc(CC3CCN(C)CC3)c(C(F)(F)F)cc2=O)c1F] |
| 139 | 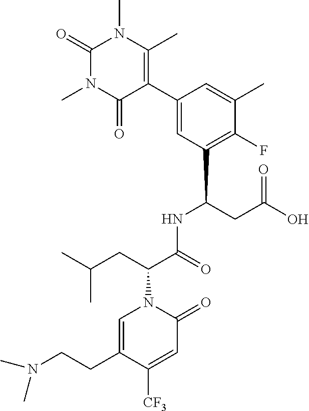 [SMILES: Cc1cc(-c2c(C)n(C)c(=O)n(C)c2=O)cc([C@@H](CC(=O)O)NC(=O)[C@@H](CC(C)C)n2cc(CCN(C)C)c(C(F)(F)F)cc2=O)c1F] | 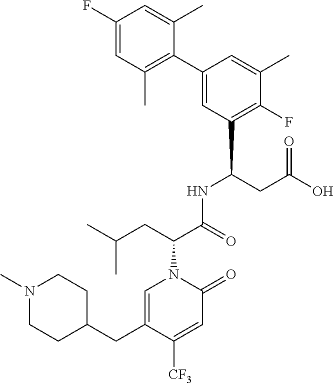 [SMILES: Cc1cc(-c2c(C)cc(F)cc2C)cc([C@@H](CC(=O)O)NC(=O)[C@@H](CC(C)C)n2cc(CC3CCN(C)CC3)c(C(F)(F)F)cc2=O)c1F] |
| 140 | 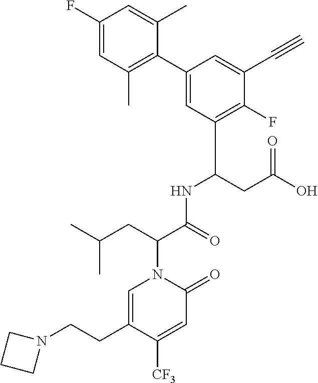 [SMILES: C#Cc1cc(-c2c(C)cc(F)cc2C)cc(C(CC(=O)O)NC(=O)C(CC(C)C)n2cc(CCN3CCC3)c(C(F)(F)F)cc2=O)c1F] | 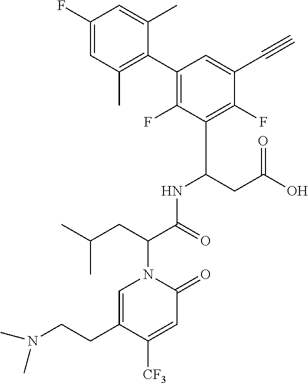 [SMILES: C#Cc1cc(-c2c(C)cc(F)cc2C)c(F)c(C(CC(=O)O)NC(=O)C(CC(C)C)n2cc(CCN(C)C)c(C(F)(F)F)cc2=O)c1F] |
| 141 | 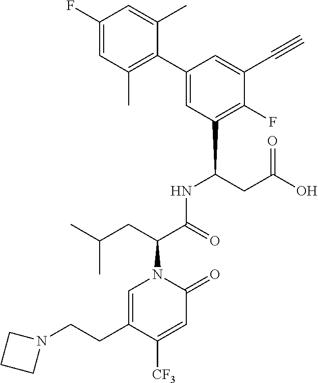 [SMILES: C#Cc1cc(-c2c(C)cc(F)cc2C)cc([C@@H](CC(=O)O)NC(=O)[C@H](CC(C)C)n2cc(CCN3CCC3)c(C(F)(F)F)cc2=O)c1F] | 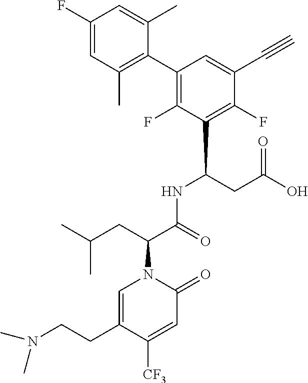 [SMILES: C#Cc1cc(-c2c(C)cc(F)cc2C)c(F)c([C@@H](CC(=O)O)NC(=O)[C@H](CC(C)C)n2cc(CCN(C)C)c(C(F)(F)F)cc2=O)c1F] |
| 142 | 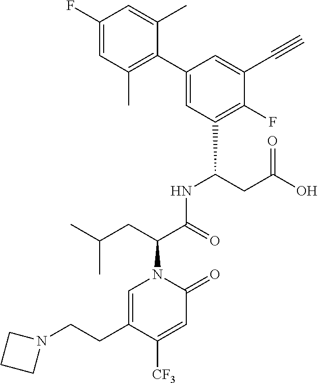 [SMILES: C#Cc1cc(-c2c(C)cc(F)cc2C)cc([C@H](CC(=O)O)NC(=O)[C@H](CC(C)C)n2cc(CCN3CCC3)c(C(F)(F)F)cc2=O)c1F] | 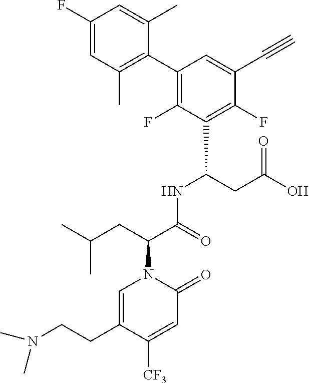 [SMILES: C#Cc1cc(-c2c(C)cc(F)cc2C)c(F)c([C@H](CC(=O)O)NC(=O)[C@H](CC(C)C)n2cc(CCN(C)C)c(C(F)(F)F)cc2=O)c1F] |
| 143 | 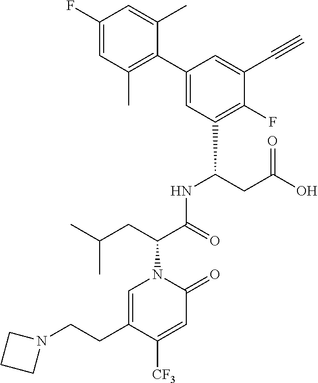 [SMILES: C#Cc1cc(-c2c(C)cc(F)cc2C)cc([C@H](CC(=O)O)NC(=O)[C@@H](CC(C)C)n2cc(CCN3CCC3)c(C(F)(F)F)cc2=O)c1F] | 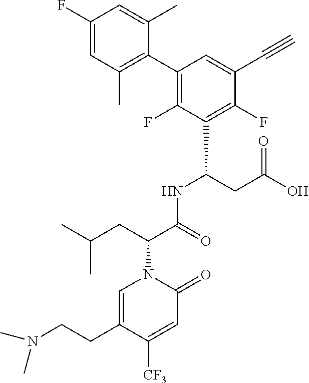 [SMILES: C#Cc1cc(-c2c(C)cc(F)cc2C)c(F)c([C@H](CC(=O)O)NC(=O)[C@@H](CC(C)C)n2cc(CCN(C)C)c(C(F)(F)F)cc2=O)c1F] |
| 144 | 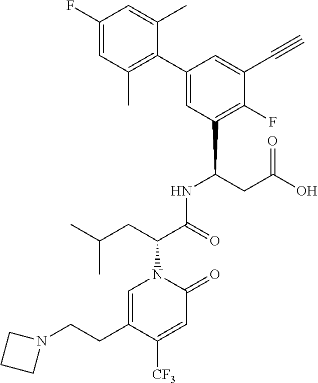 [SMILES: C#Cc1cc(-c2c(C)cc(F)cc2C)cc([C@@H](CC(=O)O)NC(=O)[C@@H](CC(C)C)n2cc(CCN3CCC3)c(C(F)(F)F)cc2=O)c1F] | 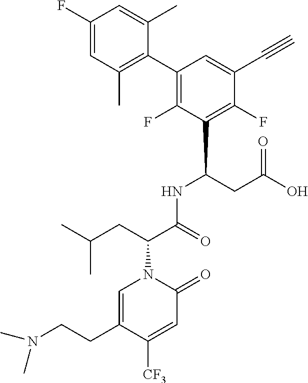 [SMILES: C#Cc1cc(-c2c(C)cc(F)cc2C)c(F)c([C@@H](CC(=O)O)NC(=O)[C@@H](CC(C)C)n2cc(CCN(C)C)c(C(F)(F)F)cc2=O)c1F] |
| 145 | 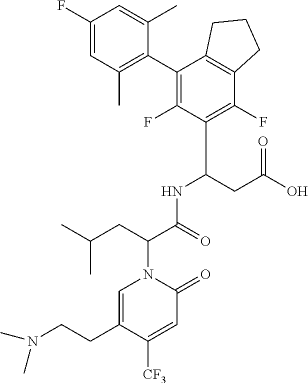 [SMILES: Cc1cc(F)cc(C)c1-c1c(F)c(C(CC(=O)O)NC(=O)C(CC(C)C)n2cc(CCN(C)C)c(C(F)(F)F)cc2=O)c(F)c2c1CCC2] | 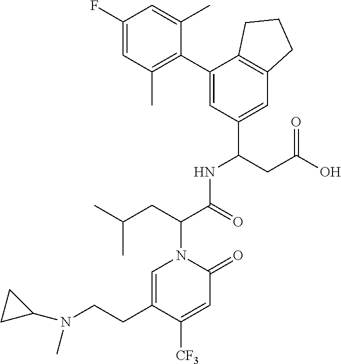 [SMILES: Cc1cc(F)cc(C)c1-c1cc(C(CC(=O)O)NC(=O)C(CC(C)C)n2cc(CCN(C)C3CC3)c(C(F)(F)F)cc2=O)cc2c1CCC2] |
| 146 | 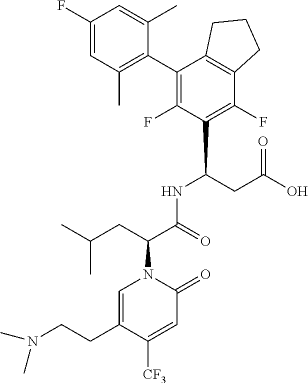 [SMILES: Cc1cc(F)cc(C)c1-c1c(F)c([C@@H](CC(=O)O)NC(=O)[C@H](CC(C)C)n2cc(CCN(C)C)c(C(F)(F)F)cc2=O)c(F)c2c1CCC2] | 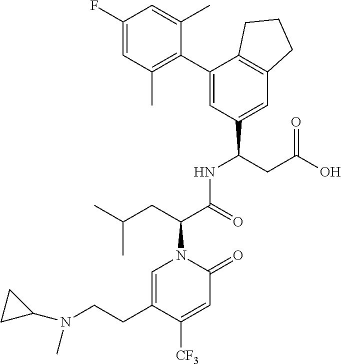 [SMILES: Cc1cc(F)cc(C)c1-c1cc([C@@H](CC(=O)O)NC(=O)[C@H](CC(C)C)n2cc(CCN(C)C3CC3)c(C(F)(F)F)cc2=O)cc2c1CCC2] |
| 147 | 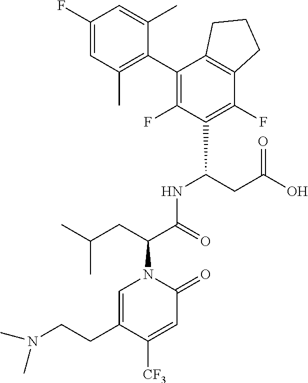 [SMILES: Cc1cc(F)cc(C)c1-c1c(F)c([C@H](CC(=O)O)NC(=O)[C@H](CC(C)C)n2cc(CCN(C)C)c(C(F)(F)F)cc2=O)c(F)c2c1CCC2] | 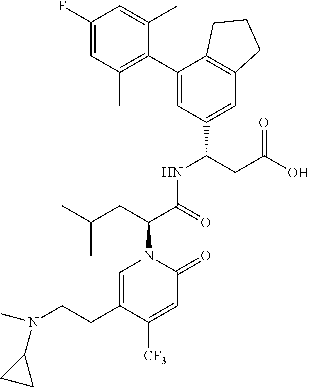 [SMILES: Cc1cc(F)cc(C)c1-c1cc([C@H](CC(=O)O)NC(=O)[C@H](CC(C)C)n2cc(CCN(C)C3CC3)c(C(F)(F)F)cc2=O)cc2c1CCC2] |
| 148 | 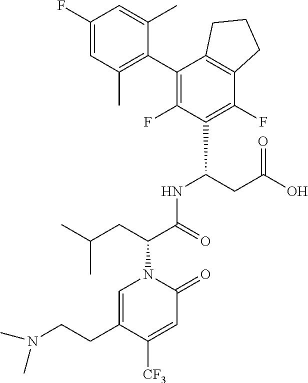 [SMILES: Cc1cc(F)cc(C)c1-c1c(F)c([C@H](CC(=O)O)NC(=O)[C@@H](CC(C)C)n2cc(CCN(C)C)c(C(F)(F)F)cc2=O)c(F)c2c1CCC2] | 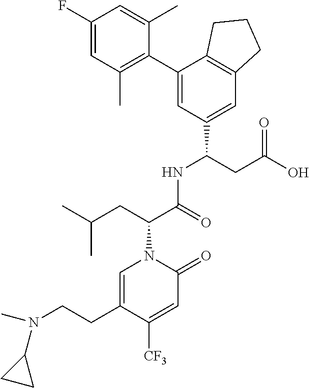 [SMILES: Cc1cc(F)cc(C)c1-c1cc([C@H](CC(=O)O)NC(=O)[C@@H](CC(C)C)n2cc(CCN(C)C3CC3)c(C(F)(F)F)cc2=O)cc2c1CCC2] |
| 149 | 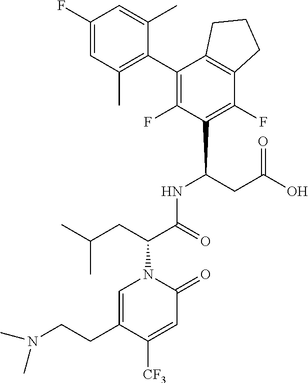 [SMILES: Cc1cc(F)cc(C)c1-c1c(F)c([C@@H](CC(=O)O)NC(=O)[C@@H](CC(C)C)n2cc(CCN(C)C)c(C(F)(F)F)cc2=O)c(F)c2c1CCC2] | 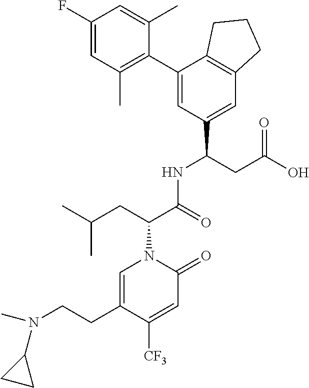 [SMILES: Cc1cc(F)cc(C)c1-c1cc([C@@H](CC(=O)O)NC(=O)[C@@H](CC(C)C)n2cc(CCN(C)C3CC3)c(C(F)(F)F)cc2=O)cc2c1CCC2] |
| 150 | 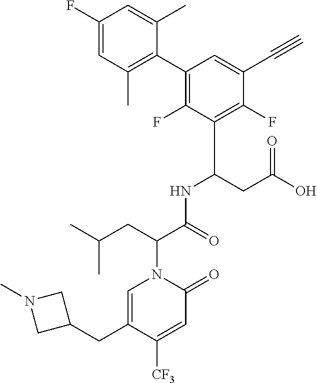 [SMILES: C#Cc1cc(-c2c(C)cc(F)cc2C)c(F)c(C(CC(=O)O)NC(=O)C(CC(C)C)n2cc(CC3CN(C)C3)c(C(F)(F)F)cc2=O)c1F] | 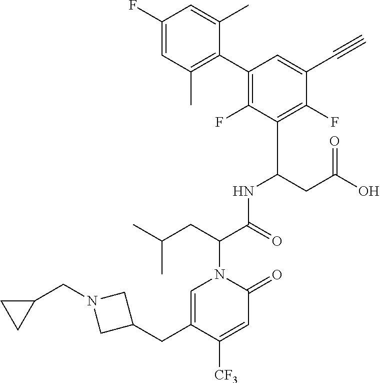 [SMILES: C#Cc1cc(-c2c(C)cc(F)cc2C)c(F)c(C(CC(=O)O)NC(=O)C(CC(C)C)n2cc(CC3CN(CC4CC4)C3)c(C(F)(F)F)cc2=O)c1F] |
| 151 | 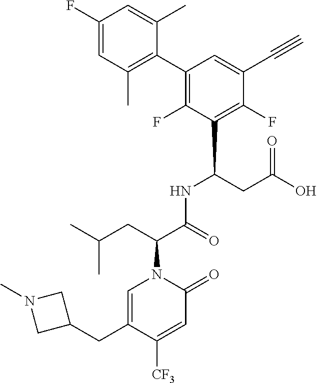 [SMILES: C#Cc1cc(-c2c(C)cc(F)cc2C)c(F)c([C@@H](CC(=O)O)NC(=O)[C@H](CC(C)C)n2cc(CC3CN(C)C3)c(C(F)(F)F)cc2=O)c1F] | 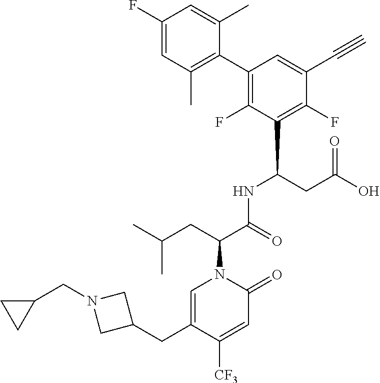 [SMILES: C#Cc1cc(-c2c(C)cc(F)cc2C)c(F)c([C@@H](CC(=O)O)NC(=O)[C@H](CC(C)C)n2cc(CC3CN(CC4CC4)C3)c(C(F)(F)F)cc2=O)c1F] |
| 152 | 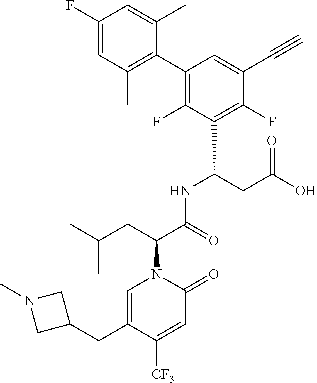 [SMILES: C#Cc1cc(-c2c(C)cc(F)cc2C)c(F)c([C@H](CC(=O)O)NC(=O)[C@H](CC(C)C)n2cc(CC3CN(C)C3)c(C(F)(F)F)cc2=O)c1F] | 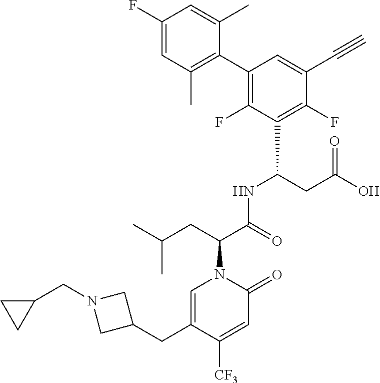 [SMILES: C#Cc1cc(-c2c(C)cc(F)cc2C)c(F)c([C@H](CC(=O)O)NC(=O)[C@H](CC(C)C)n2cc(CC3CN(CC4CC4)C3)c(C(F)(F)F)cc2=O)c1F] |
| 153 | 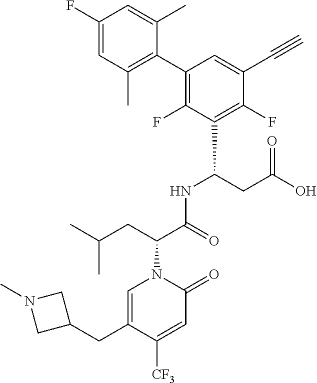 [SMILES: C#Cc1cc(-c2c(C)cc(F)cc2C)c(F)c([C@H](CC(=O)O)NC(=O)[C@@H](CC(C)C)n2cc(CC3CN(C)C3)c(C(F)(F)F)cc2=O)c1F] | 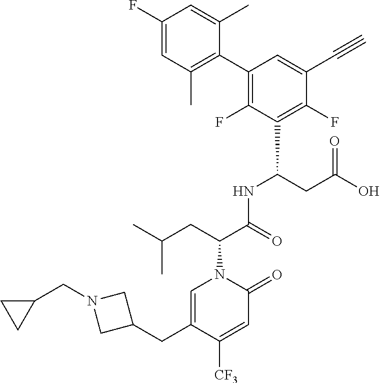 [SMILES: C#Cc1cc(-c2c(C)cc(F)cc2C)c(F)c([C@H](CC(=O)O)NC(=O)[C@@H](CC(C)C)n2cc(CC3CN(CC4CC4)C3)c(C(F)(F)F)cc2=O)c1F] |
| 154 | 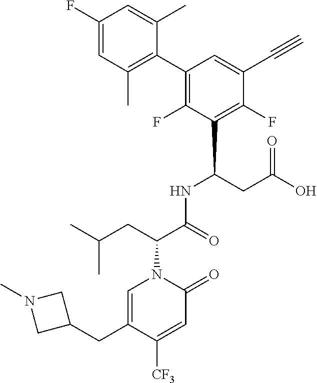 [SMILES: C#Cc1cc(-c2c(C)cc(F)cc2C)c(F)c([C@@H](CC(=O)O)NC(=O)[C@@H](CC(C)C)n2cc(CC3CN(C)C3)c(C(F)(F)F)cc2=O)c1F] | 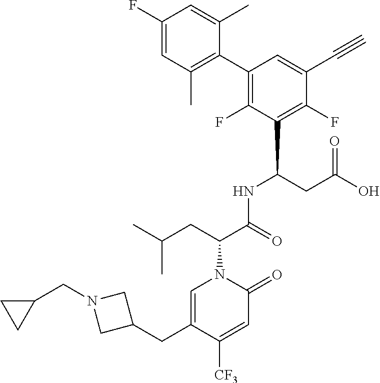 [SMILES: C#Cc1cc(-c2c(C)cc(F)cc2C)c(F)c([C@@H](CC(=O)O)NC(=O)[C@@H](CC(C)C)n2cc(CC3CN(CC4CC4)C3)c(C(F)(F)F)cc2=O)c1F] |
| 155 | 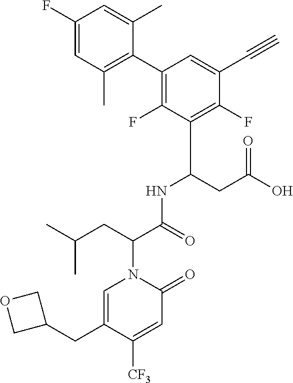 [SMILES: C#Cc1cc(-c2c(C)cc(F)cc2C)c(F)c(C(CC(=O)O)NC(=O)C(CC(C)C)n2cc(CC3COC3)c(C(F)(F)F)cc2=O)c1F] | 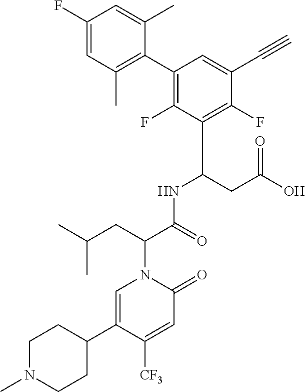 [SMILES: C#Cc1cc(-c2c(C)cc(F)cc2C)c(F)c(C(CC(=O)O)NC(=O)C(CC(C)C)n2cc(C3CCN(C)CC3)c(C(F)(F)F)cc2=O)c1F] |
| 156 | 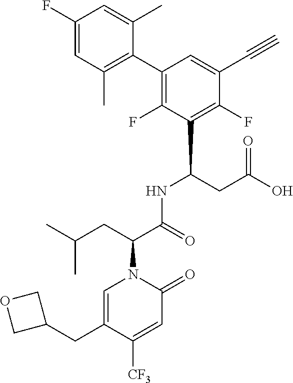 [SMILES: C#Cc1cc(-c2c(C)cc(F)cc2C)c(F)c([C@@H](CC(=O)O)NC(=O)[C@H](CC(C)C)n2cc(CC3COC3)c(C(F)(F)F)cc2=O)c1F] | 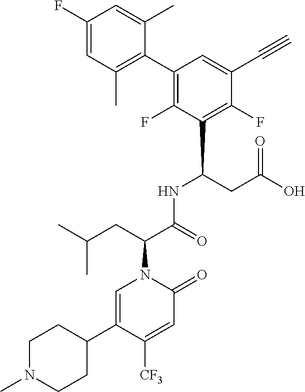 [SMILES: C#Cc1cc(-c2c(C)cc(F)cc2C)c(F)c([C@@H](CC(=O)O)NC(=O)[C@H](CC(C)C)n2cc(C3CCN(C)CC3)c(C(F)(F)F)cc2=O)c1F] |
| 157 | 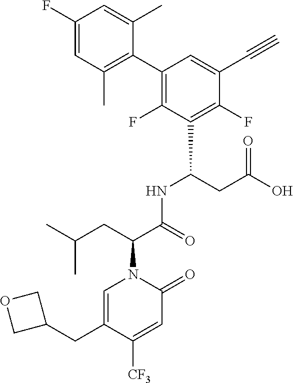 [SMILES: C#Cc1cc(-c2c(C)cc(F)cc2C)c(F)c([C@H](CC(=O)O)NC(=O)[C@H](CC(C)C)n2cc(CC3COC3)c(C(F)(F)F)cc2=O)c1F] | 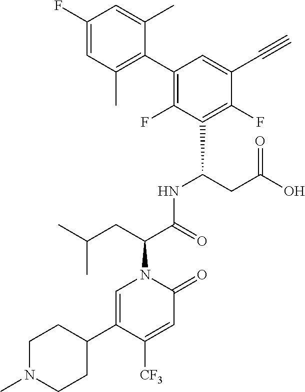 [SMILES: C#Cc1cc(-c2c(C)cc(F)cc2C)c(F)c([C@H](CC(=O)O)NC(=O)[C@H](CC(C)C)n2cc(C3CCN(C)CC3)c(C(F)(F)F)cc2=O)c1F] |
| 158 | 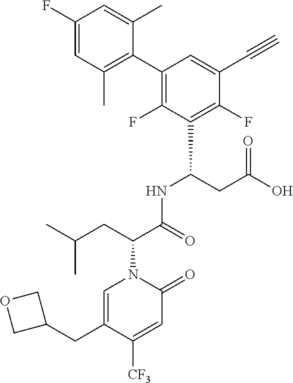 [SMILES: C#Cc1cc(-c2c(C)cc(F)cc2C)c(F)c([C@H](CC(=O)O)NC(=O)[C@@H](CC(C)C)n2cc(CC3COC3)c(C(F)(F)F)cc2=O)c1F] | 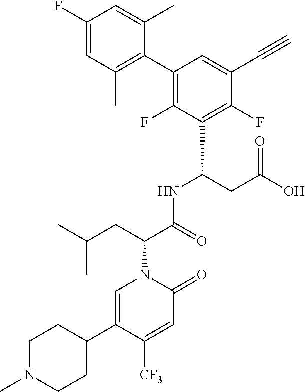 [SMILES: C#Cc1cc(-c2c(C)cc(F)cc2C)c(F)c([C@H](CC(=O)O)NC(=O)[C@@H](CC(C)C)n2cc(C3CCN(C)CC3)c(C(F)(F)F)cc2=O)c1F] |
| 159 | 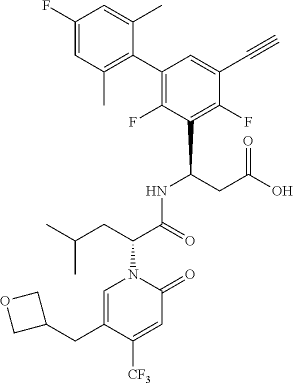 [SMILES: C#Cc1cc(-c2c(C)cc(F)cc2C)c(F)c([C@@H](CC(=O)O)NC(=O)[C@@H](CC(C)C)n2cc(CC3COC3)c(C(F)(F)F)cc2=O)c1F] | 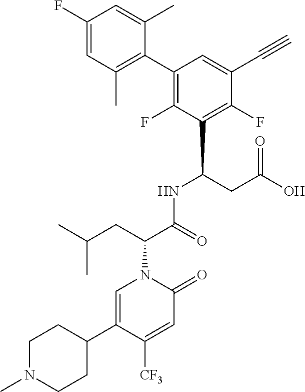 [SMILES: C#Cc1cc(-c2c(C)cc(F)cc2C)c(F)c([C@@H](CC(=O)O)NC(=O)[C@@H](CC(C)C)n2cc(C3CCN(C)CC3)c(C(F)(F)F)cc2=O)c1F] |
| 160 | 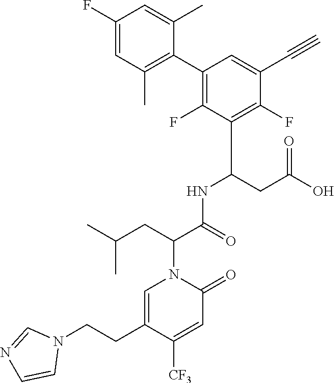 [SMILES: C#Cc1cc(-c2c(C)cc(F)cc2C)c(F)c(C(CC(=O)O)NC(=O)C(CC(C)C)n2cc(CCn3ccnc3)c(C(F)(F)F)cc2=O)c1F] | 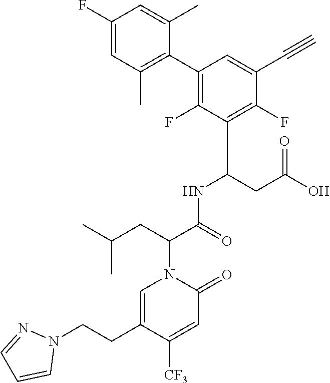 [SMILES: C#Cc1cc(-c2c(C)cc(F)cc2C)c(F)c(C(CC(=O)O)NC(=O)C(CC(C)C)n2cc(CCn3cccn3)c(C(F)(F)F)cc2=O)c1F] |
| 161 | 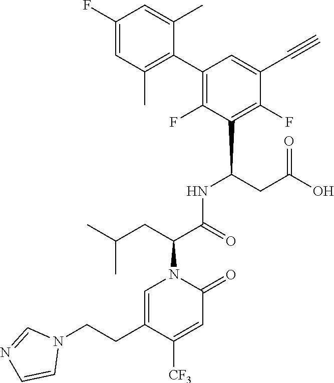 [SMILES: C#Cc1cc(-c2c(C)cc(F)cc2C)c(F)c([C@@H](CC(=O)O)NC(=O)[C@H](CC(C)C)n2cc(CCn3ccnc3)c(C(F)(F)F)cc2=O)c1F] | 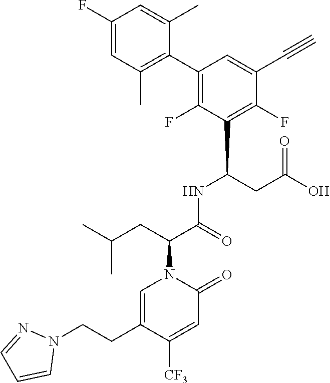 [SMILES: C#Cc1cc(-c2c(C)cc(F)cc2C)c(F)c([C@@H](CC(=O)O)NC(=O)[C@H](CC(C)C)n2cc(CCn3cccn3)c(C(F)(F)F)cc2=O)c1F] |
| 162 | 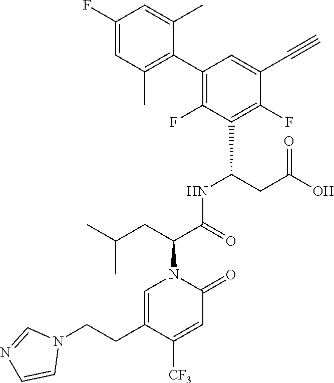 [SMILES: C#Cc1cc(-c2c(C)cc(F)cc2C)c(F)c([C@H](CC(=O)O)NC(=O)[C@H](CC(C)C)n2cc(CCn3ccnc3)c(C(F)(F)F)cc2=O)c1F] | 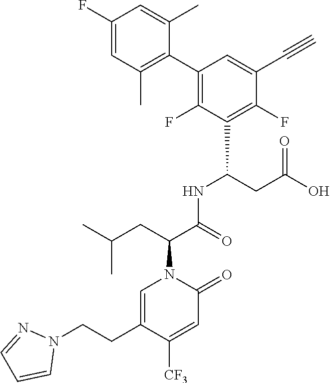 [SMILES: C#Cc1cc(-c2c(C)cc(F)cc2C)c(F)c([C@H](CC(=O)O)NC(=O)[C@H](CC(C)C)n2cc(CCn3cccn3)c(C(F)(F)F)cc2=O)c1F] |
| 163 | 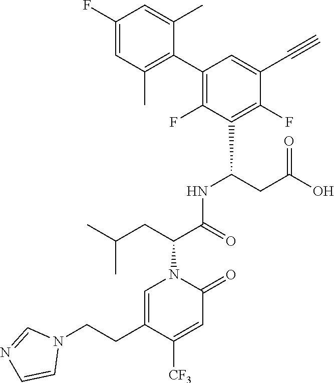 [SMILES: C#Cc1cc(-c2c(C)cc(F)cc2C)c(F)c([C@H](CC(=O)O)NC(=O)[C@@H](CC(C)C)n2cc(CCn3ccnc3)c(C(F)(F)F)cc2=O)c1F] | 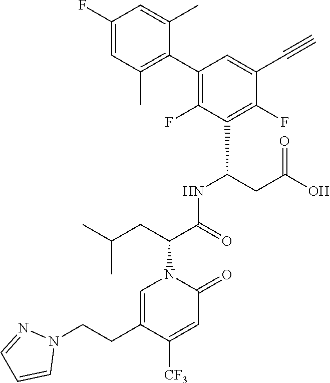 [SMILES: C#Cc1cc(-c2c(C)cc(F)cc2C)c(F)c([C@H](CC(=O)O)NC(=O)[C@@H](CC(C)C)n2cc(CCn3cccn3)c(C(F)(F)F)cc2=O)c1F] |
| 164 | 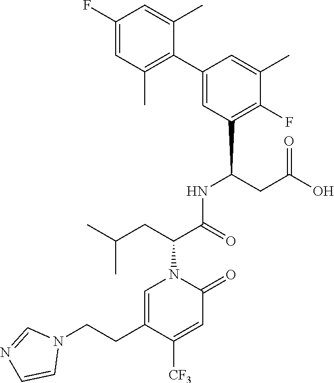 [SMILES: Cc1cc(-c2c(C)cc(F)cc2C)cc([C@@H](CC(=O)O)NC(=O)[C@@H](CC(C)C)n2cc(CCn3ccnc3)c(C(F)(F)F)cc2=O)c1F] | 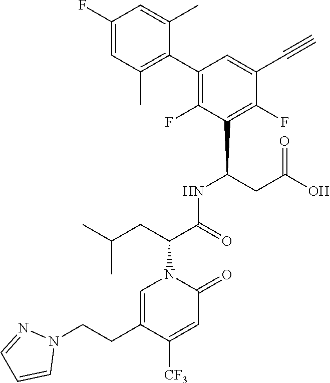 [SMILES: C#Cc1cc(-c2c(C)cc(F)cc2C)c(F)c([C@@H](CC(=O)O)NC(=O)[C@@H](CC(C)C)n2cc(CCn3cccn3)c(C(F)(F)F)cc2=O)c1F] |
| 165 | 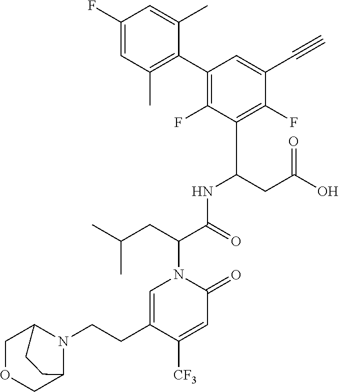 [SMILES: C#Cc1cc(-c2c(C)cc(F)cc2C)c(F)c(C(CC(=O)O)NC(=O)C(CC(C)C)n2cc(CCN3C4CCC3COC4)c(C(F)(F)F)cc2=O)c1F] | 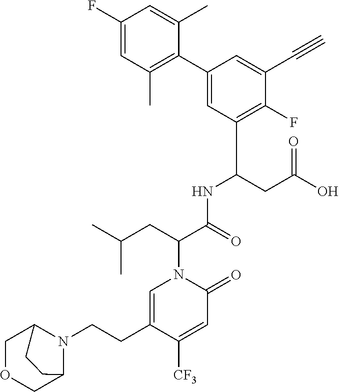 [SMILES: C#Cc1cc(-c2c(C)cc(F)cc2C)cc(C(CC(=O)O)NC(=O)C(CC(C)C)n2cc(CCN3C4CCC3COC4)c(C(F)(F)F)cc2=O)c1F] |
| 166 | 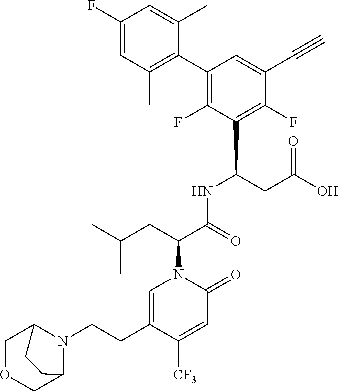 [SMILES: C#Cc1cc(-c2c(C)cc(F)cc2C)c(F)c([C@@H](CC(=O)O)NC(=O)[C@H](CC(C)C)n2cc(CCN3C4CCC3COC4)c(C(F)(F)F)cc2=O)c1F] | 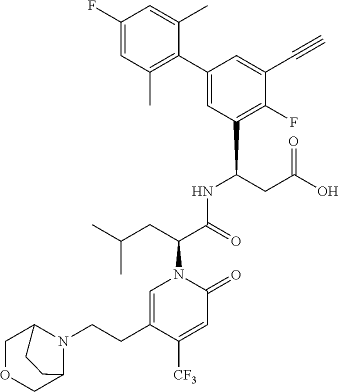 [SMILES: C#Cc1cc(-c2c(C)cc(F)cc2C)cc([C@@H](CC(=O)O)NC(=O)[C@H](CC(C)C)n2cc(CCN3C4CCC3COC4)c(C(F)(F)F)cc2=O)c1F] |
| 167 | 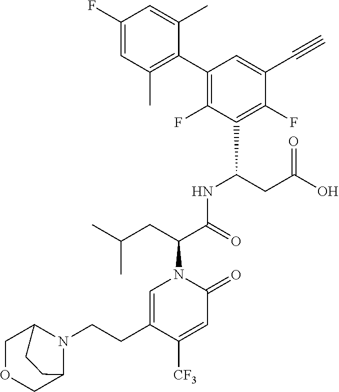 [SMILES: C#Cc1cc(-c2c(C)cc(F)cc2C)c(F)c([C@H](CC(=O)O)NC(=O)[C@H](CC(C)C)n2cc(CCN3C4CCC3COC4)c(C(F)(F)F)cc2=O)c1F] | 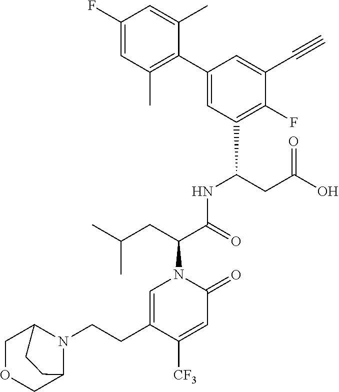 [SMILES: C#Cc1cc(-c2c(C)cc(F)cc2C)cc([C@H](CC(=O)O)NC(=O)[C@H](CC(C)C)n2cc(CCN3C4CCC3COC4)c(C(F)(F)F)cc2=O)c1F] |
| 168 | 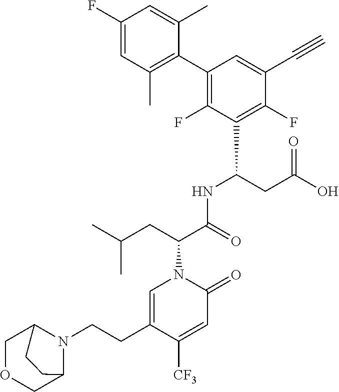 [SMILES: C#Cc1cc(-c2c(C)cc(F)cc2C)c(F)c([C@H](CC(=O)O)NC(=O)[C@@H](CC(C)C)n2cc(CCN3C4CCC3COC4)c(C(F)(F)F)cc2=O)c1F] | 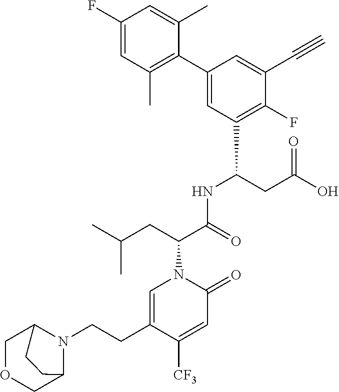 [SMILES: C#Cc1cc(-c2c(C)cc(F)cc2C)cc([C@H](CC(=O)O)NC(=O)[C@@H](CC(C)C)n2cc(CCN3C4CCC3COC4)c(C(F)(F)F)cc2=O)c1F] |
| 169 | 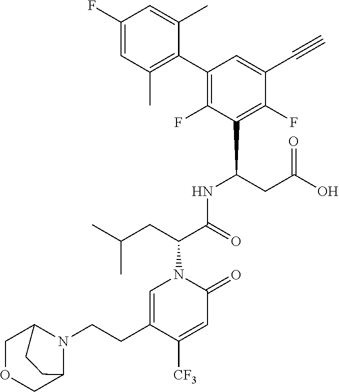 [SMILES: C#Cc1cc(-c2c(C)cc(F)cc2C)c(F)c([C@@H](CC(=O)O)NC(=O)[C@@H](CC(C)C)n2cc(CCN3C4CCC3COC4)c(C(F)(F)F)cc2=O)c1F] | 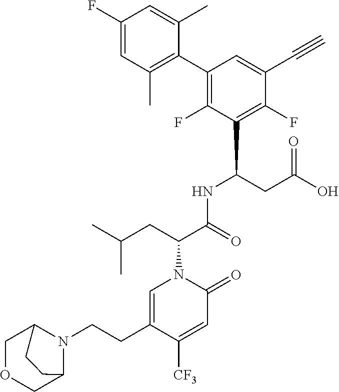 [SMILES: C#Cc1cc(-c2c(C)cc(F)cc2C)c(F)c([C@@H](CC(=O)O)NC(=O)[C@@H](CC(C)C)n2cc(CCN3C4CCC3COC4)c(C(F)(F)F)cc2=O)c1F] |
| 170 | 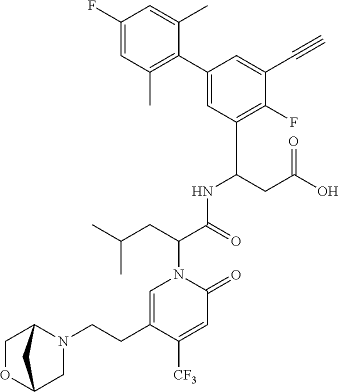 [SMILES: C#Cc1cc(-c2c(C)cc(F)cc2C)cc(C(CC(=O)O)NC(=O)C(CC(C)C)n2cc(CCN3C[C@H]4C[C@@H]3CO4)c(C(F)(F)F)cc2=O)c1F] | 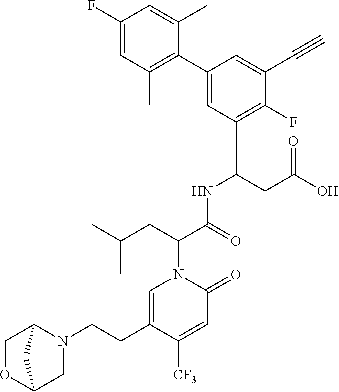 [SMILES: C#Cc1cc(-c2c(C)cc(F)cc2C)cc(C(CC(=O)O)NC(=O)C(CC(C)C)n2cc(CCN3C[C@@H]4C[C@H]3CO4)c(C(F)(F)F)cc2=O)c1F] |
| 171 | 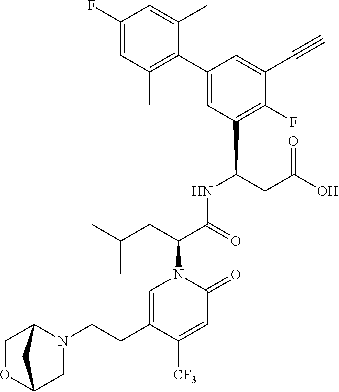 [SMILES: C#Cc1cc(-c2c(C)cc(F)cc2C)cc([C@@H](CC(=O)O)NC(=O)[C@H](CC(C)C)n2cc(CCN3C[C@H]4C[C@@H]3CO4)c(C(F)(F)F)cc2=O)c1F] | 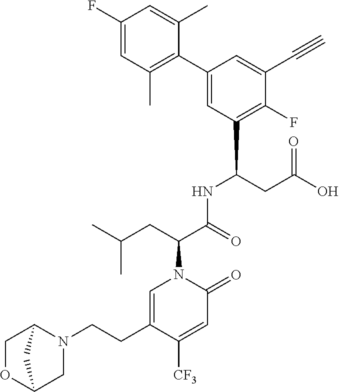 [SMILES: C#Cc1cc(-c2c(C)cc(F)cc2C)cc([C@@H](CC(=O)O)NC(=O)[C@H](CC(C)C)n2cc(CCN3C[C@@H]4C[C@H]3CO4)c(C(F)(F)F)cc2=O)c1F] |
| 172 | 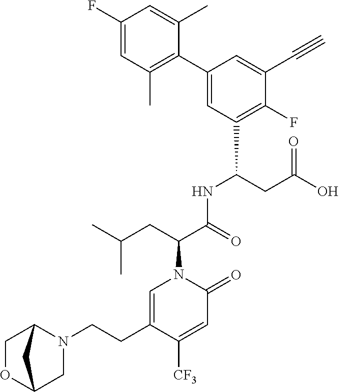 [SMILES: C#Cc1cc(-c2c(C)cc(F)cc2C)cc([C@H](CC(=O)O)NC(=O)[C@H](CC(C)C)n2cc(CCN3C[C@H]4C[C@@H]3CO4)c(C(F)(F)F)cc2=O)c1F] | 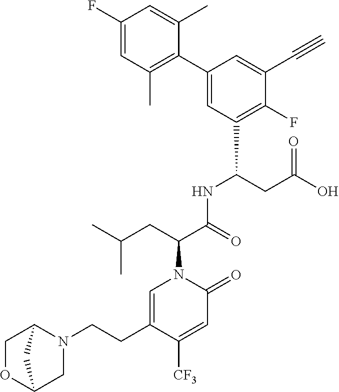 [SMILES: C#Cc1cc(-c2c(C)cc(F)cc2C)cc([C@H](CC(=O)O)NC(=O)[C@H](CC(C)C)n2cc(CCN3C[C@@H]4C[C@H]3CO4)c(C(F)(F)F)cc2=O)c1F] |
| 173 | 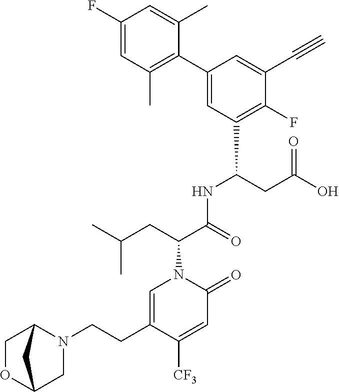 [SMILES: C#Cc1cc(-c2c(C)cc(F)cc2C)cc([C@H](CC(=O)O)NC(=O)[C@@H](CC(C)C)n2cc(CCN3C[C@H]4C[C@@H]3CO4)c(C(F)(F)F)cc2=O)c1F] | 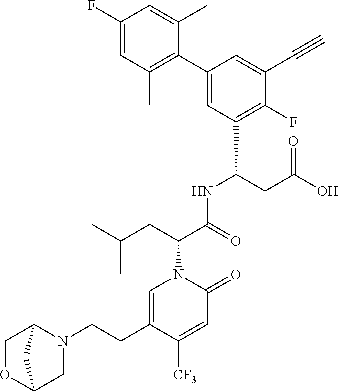 [SMILES: C#Cc1cc(-c2c(C)cc(F)cc2C)cc([C@H](CC(=O)O)NC(=O)[C@@H](CC(C)C)n2cc(CCN3C[C@@H]4C[C@H]3CO4)c(C(F)(F)F)cc2=O)c1F] |
| 174 | 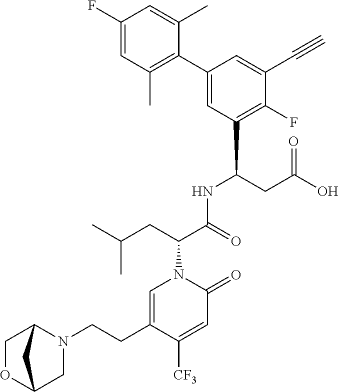 [SMILES: C#Cc1cc(-c2c(C)cc(F)cc2C)cc([C@@H](CC(=O)O)NC(=O)[C@@H](CC(C)C)n2cc(CCN3C[C@H]4C[C@@H]3CO4)c(C(F)(F)F)cc2=O)c1F] | 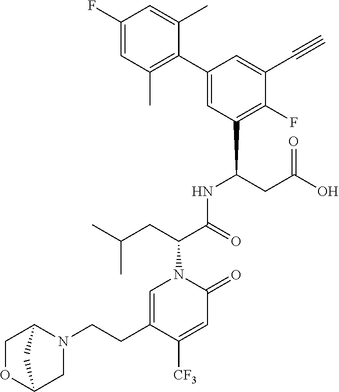 [SMILES: C#Cc1cc(-c2c(C)cc(F)cc2C)cc([C@@H](CC(=O)O)NC(=O)[C@@H](CC(C)C)n2cc(CCN3C[C@@H]4C[C@H]3CO4)c(C(F)(F)F)cc2=O)c1F] |
| 175 | 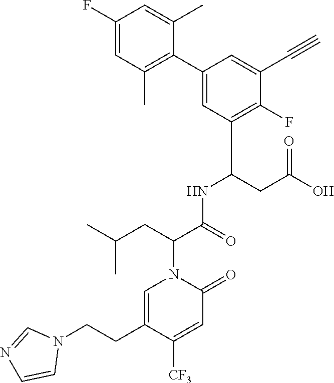 [SMILES: C#Cc1cc(-c2c(C)cc(F)cc2C)cc(C(CC(=O)O)NC(=O)C(CC(C)C)n2cc(CCn3ccnc3)c(C(F)(F)F)cc2=O)c1F] | 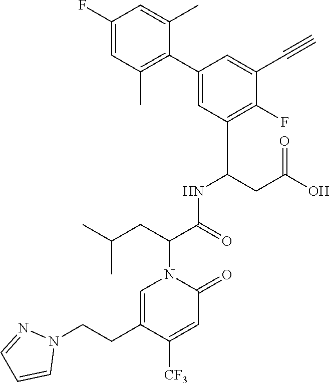 [SMILES: C#Cc1cc(-c2c(C)cc(F)cc2C)cc(C(CC(=O)O)NC(=O)C(CC(C)C)n2cc(CCn3cccn3)c(C(F)(F)F)cc2=O)c1F] |
| 176 | 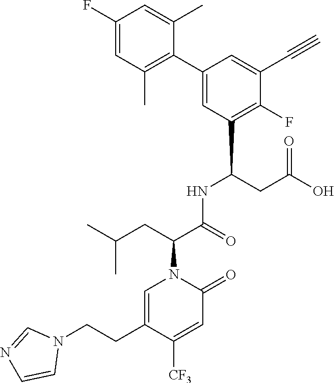 [SMILES: C#Cc1cc(-c2c(C)cc(F)cc2C)cc([C@@H](CC(=O)O)NC(=O)[C@H](CC(C)C)n2cc(CCn3ccnc3)c(C(F)(F)F)cc2=O)c1F] | 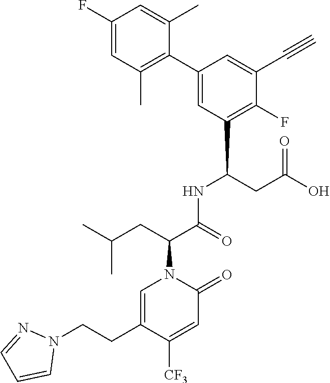 [SMILES: C#Cc1cc(-c2c(C)cc(F)cc2C)cc([C@@H](CC(=O)O)NC(=O)[C@H](CC(C)C)n2cc(CCn3cccn3)c(C(F)(F)F)cc2=O)c1F] |
| 177 | 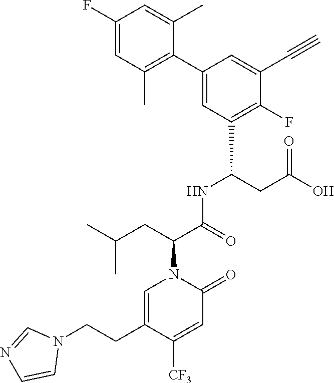 [SMILES: C#Cc1cc(-c2c(C)cc(F)cc2C)cc([C@H](CC(=O)O)NC(=O)[C@H](CC(C)C)n2cc(CCn3ccnc3)c(C(F)(F)F)cc2=O)c1F] | 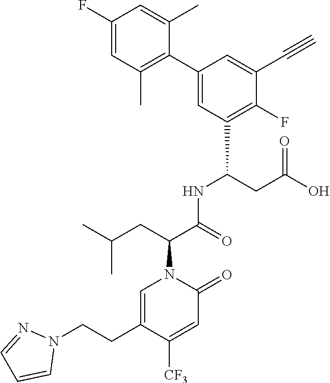 [SMILES: C#Cc1cc(-c2c(C)cc(F)cc2C)cc([C@H](CC(=O)O)NC(=O)[C@H](CC(C)C)n2cc(CCn3cccn3)c(C(F)(F)F)cc2=O)c1F] |
| 178 | 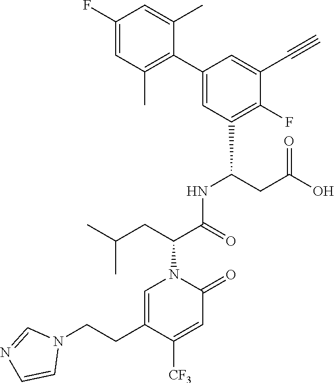 [SMILES: C#Cc1cc(-c2c(C)cc(F)cc2C)cc([C@H](CC(=O)O)NC(=O)[C@@H](CC(C)C)n2cc(CCn3ccnc3)c(C(F)(F)F)cc2=O)c1F] | 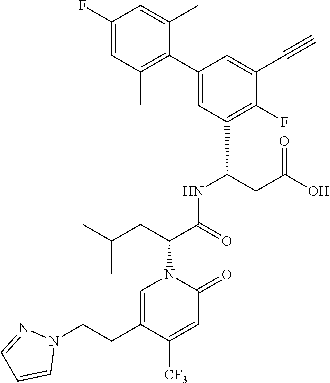 [SMILES: C#Cc1cc(-c2c(C)cc(F)cc2C)cc([C@H](CC(=O)O)NC(=O)[C@@H](CC(C)C)n2cc(CCn3cccn3)c(C(F)(F)F)cc2=O)c1F] |
| 179 | 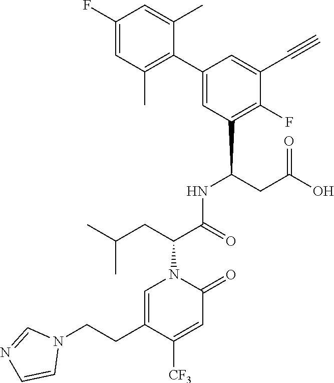 [SMILES: C#Cc1cc(-c2c(C)cc(F)cc2C)cc([C@@H](CC(=O)O)NC(=O)[C@@H](CC(C)C)n2cc(CCn3ccnc3)c(C(F)(F)F)cc2=O)c1F] | 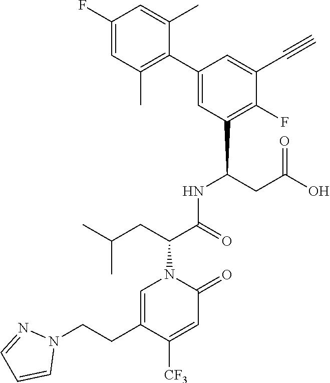 [SMILES: C#Cc1cc(-c2c(C)cc(F)cc2C)cc([C@@H](CC(=O)O)NC(=O)[C@@H](CC(C)C)n2cc(CCn3cccn3)c(C(F)(F)F)cc2=O)c1F] |
| 180 | 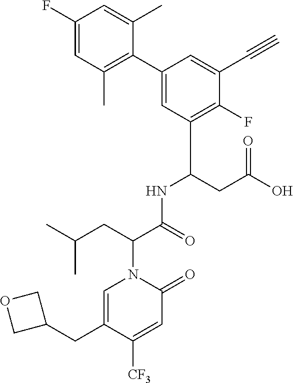 [SMILES: C#Cc1cc(-c2c(C)cc(F)cc2C)cc(C(CC(=O)O)NC(=O)C(CC(C)C)n2cc(CC3COC3)c(C(F)(F)F)cc2=O)c1F] | 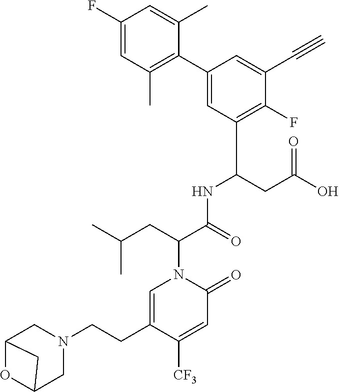 [SMILES: C#Cc1cc(-c2c(C)cc(F)cc2C)cc(C(CC(=O)O)NC(=O)C(CC(C)C)n2cc(CCN3CC4CC(C3)O4)c(C(F)(F)F)cc2=O)c1F] |
| 181 | 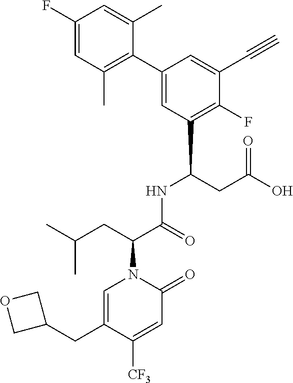 [SMILES: C#Cc1cc(-c2c(C)cc(F)cc2C)cc([C@@H](CC(=O)O)NC(=O)[C@H](CC(C)C)n2cc(CC3COC3)c(C(F)(F)F)cc2=O)c1F] | 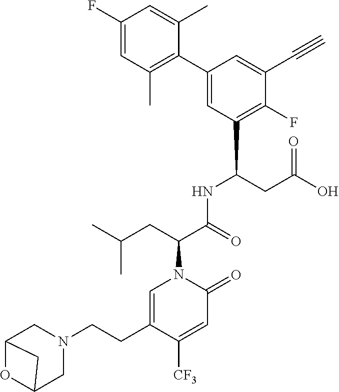 [SMILES: C#Cc1cc(-c2c(C)cc(F)cc2C)cc([C@@H](CC(=O)O)NC(=O)[C@H](CC(C)C)n2cc(CCN3CC4CC(C3)O4)c(C(F)(F)F)cc2=O)c1F] |
| 182 | 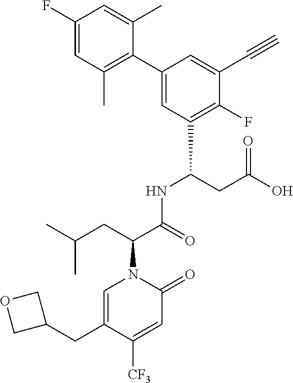 [SMILES: C#Cc1cc(-c2c(C)cc(F)cc2C)cc([C@H](CC(=O)O)NC(=O)[C@H](CC(C)C)n2cc(CC3COC3)c(C(F)(F)F)cc2=O)c1F] | 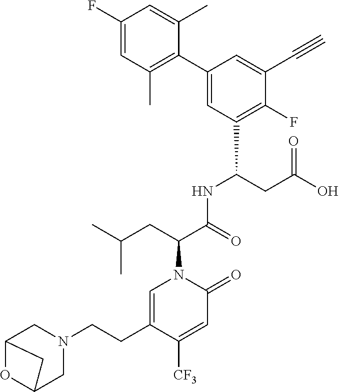 [SMILES: C#Cc1cc(-c2c(C)cc(F)cc2C)cc([C@H](CC(=O)O)NC(=O)[C@H](CC(C)C)n2cc(CCN3CC4CC(C3)O4)c(C(F)(F)F)cc2=O)c1F] |
| 183 | 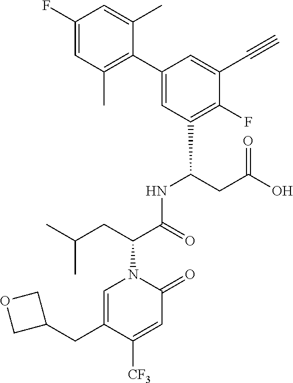 [SMILES: C#Cc1cc(-c2c(C)cc(F)cc2C)cc([C@H](CC(=O)O)NC(=O)[C@@H](CC(C)C)n2cc(CC3COC3)c(C(F)(F)F)cc2=O)c1F] | 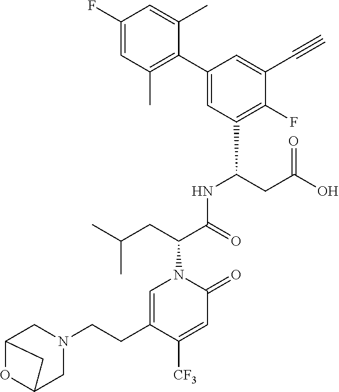 [SMILES: C#Cc1cc(-c2c(C)cc(F)cc2C)cc([C@H](CC(=O)O)NC(=O)[C@@H](CC(C)C)n2cc(CCN3CC4CC(C3)O4)c(C(F)(F)F)cc2=O)c1F] |
| 184 | 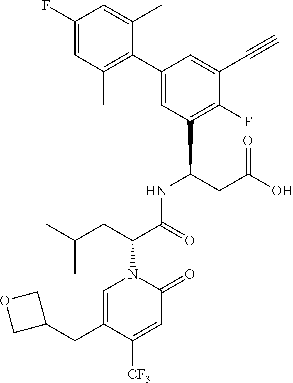 [SMILES: C#Cc1cc(-c2c(C)cc(F)cc2C)cc([C@@H](CC(=O)O)NC(=O)[C@@H](CC(C)C)n2cc(CC3COC3)c(C(F)(F)F)cc2=O)c1F] | 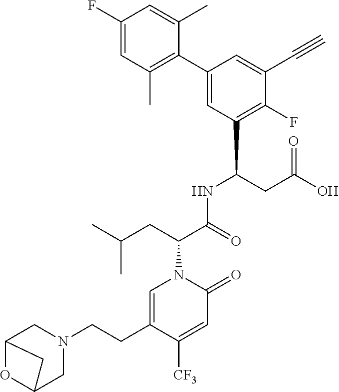 [SMILES: C#Cc1cc(-c2c(C)cc(F)cc2C)cc([C@@H](CC(=O)O)NC(=O)[C@@H](CC(C)C)n2cc(CCN3CC4CC(C3)O4)c(C(F)(F)F)cc2=O)c1F] |
| 185 | 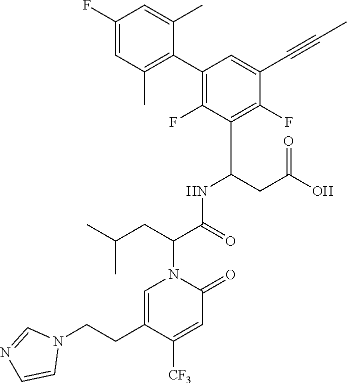 [SMILES: CC#Cc1cc(-c2c(C)cc(F)cc2C)c(F)c(C(CC(=O)O)NC(=O)C(CC(C)C)n2cc(CCn3ccnc3)c(C(F)(F)F)cc2=O)c1F] | 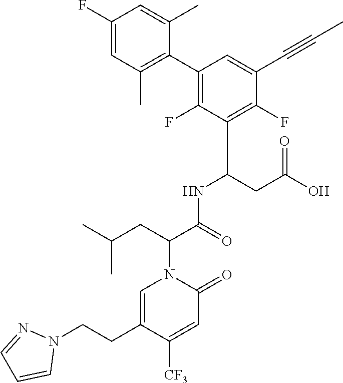 [SMILES: CC#Cc1cc(-c2c(C)cc(F)cc2C)c(F)c(C(CC(=O)O)NC(=O)C(CC(C)C)n2cc(CCn3cccn3)c(C(F)(F)F)cc2=O)c1F] |
| 186 | 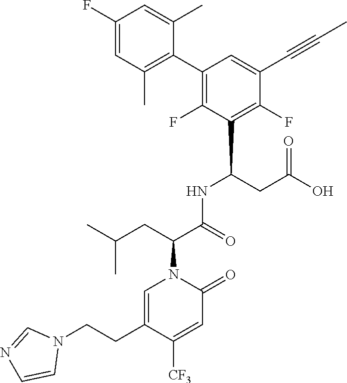 [SMILES: CC#Cc1cc(-c2c(C)cc(F)cc2C)c(F)c([C@@H](CC(=O)O)NC(=O)[C@H](CC(C)C)n2cc(CCn3ccnc3)c(C(F)(F)F)cc2=O)c1F] | 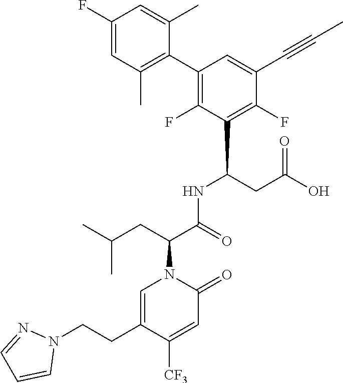 [SMILES: CC#Cc1cc(-c2c(C)cc(F)cc2C)c(F)c([C@@H](CC(=O)O)NC(=O)[C@H](CC(C)C)n2cc(CCn3cccn3)c(C(F)(F)F)cc2=O)c1F] |
| 187 | 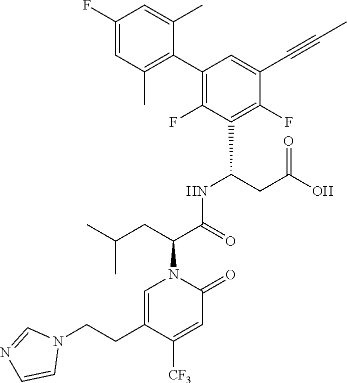 [SMILES: CC#Cc1cc(-c2c(C)cc(F)cc2C)c(F)c([C@H](CC(=O)O)NC(=O)[C@H](CC(C)C)n2cc(CCn3ccnc3)c(C(F)(F)F)cc2=O)c1F] | 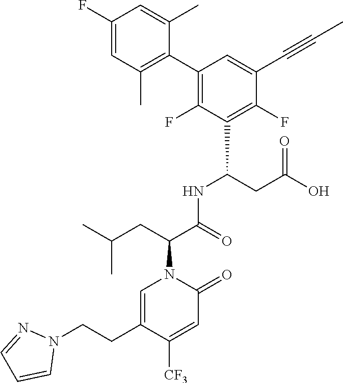 [SMILES: CC#Cc1cc(-c2c(C)cc(F)cc2C)c(F)c([C@H](CC(=O)O)NC(=O)[C@H](CC(C)C)n2cc(CCn3cccn3)c(C(F)(F)F)cc2=O)c1F] |
| 188 | 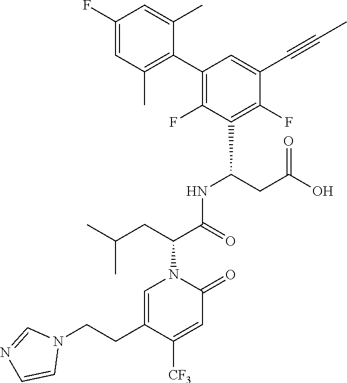 [SMILES: CC#Cc1cc(-c2c(C)cc(F)cc2C)c(F)c([C@H](CC(=O)O)NC(=O)[C@@H](CC(C)C)n2cc(CCn3ccnc3)c(C(F)(F)F)cc2=O)c1F] | 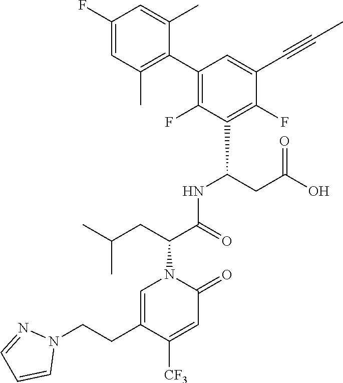 [SMILES: CC#Cc1cc(-c2c(C)cc(F)cc2C)c(F)c([C@H](CC(=O)O)NC(=O)[C@@H](CC(C)C)n2cc(CCn3cccn3)c(C(F)(F)F)cc2=O)c1F] |
| 189 | 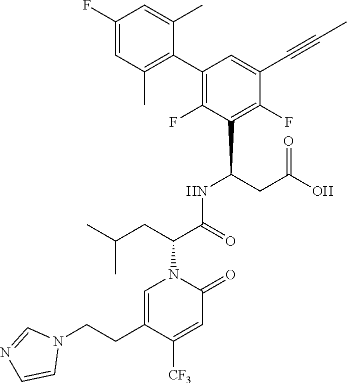 [SMILES: CC#Cc1cc(-c2c(C)cc(F)cc2C)c(F)c([C@@H](CC(=O)O)NC(=O)[C@@H](CC(C)C)n2cc(CCn3ccnc3)c(C(F)(F)F)cc2=O)c1F] | 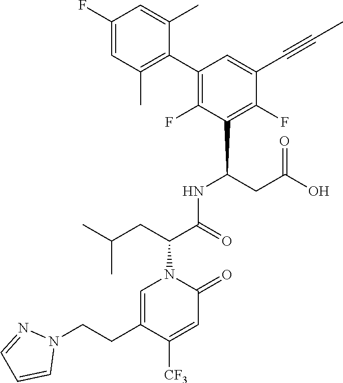 [SMILES: CC#Cc1cc(-c2c(C)cc(F)cc2C)c(F)c([C@@H](CC(=O)O)NC(=O)[C@@H](CC(C)C)n2cc(CCn3cccn3)c(C(F)(F)F)cc2=O)c1F] |
| 190 | 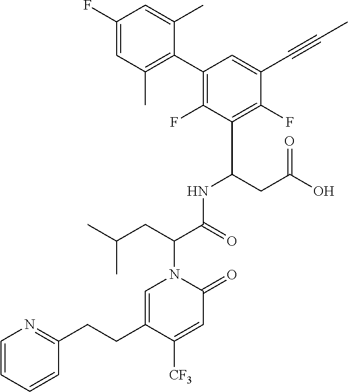 [SMILES: CC#Cc1cc(-c2c(C)cc(F)cc2C)c(F)c(C(CC(=O)O)NC(=O)C(CC(C)C)n2cc(CCc3ccccn3)c(C(F)(F)F)cc2=O)c1F] | 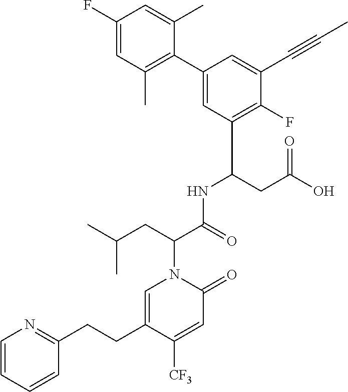 [SMILES: CC#Cc1cc(-c2c(C)cc(F)cc2C)cc(C(CC(=O)O)NC(=O)C(CC(C)C)n2cc(CCc3ccccn3)c(C(F)(F)F)cc2=O)c1F] |
| 191 | 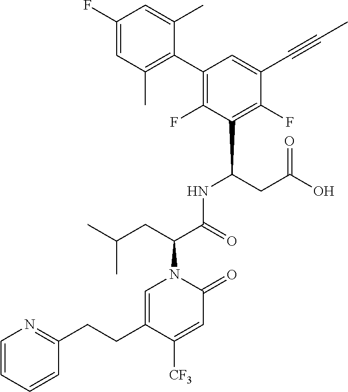 [SMILES: CC#Cc1cc(-c2c(C)cc(F)cc2C)c(F)c([C@@H](CC(=O)O)NC(=O)[C@H](CC(C)C)n2cc(CCc3ccccn3)c(C(F)(F)F)cc2=O)c1F] | 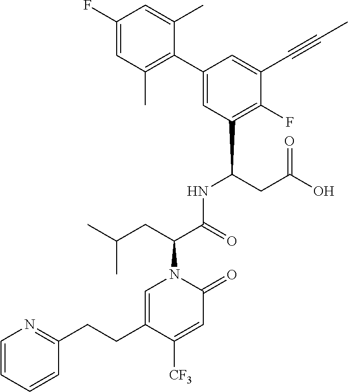 [SMILES: CC#Cc1cc(-c2c(C)cc(F)cc2C)cc([C@@H](CC(=O)O)NC(=O)[C@H](CC(C)C)n2cc(CCc3ccccn3)c(C(F)(F)F)cc2=O)c1F] |
| 192 | 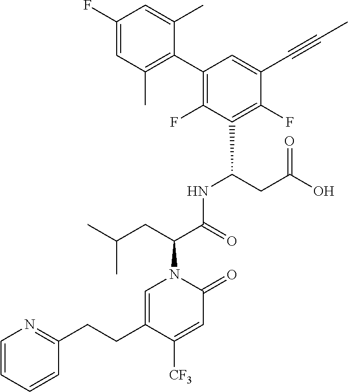 [SMILES: CC#Cc1cc(-c2c(C)cc(F)cc2C)c(F)c([C@H](CC(=O)O)NC(=O)[C@H](CC(C)C)n2cc(CCc3ccccn3)c(C(F)(F)F)cc2=O)c1F] | 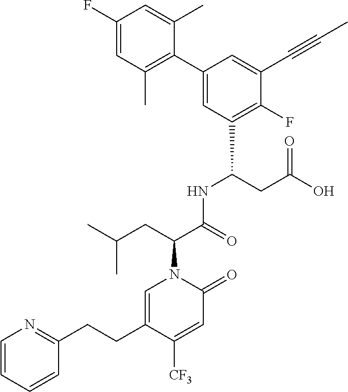 [SMILES: CC#Cc1cc(-c2c(C)cc(F)cc2C)cc([C@H](CC(=O)O)NC(=O)[C@H](CC(C)C)n2cc(CCc3ccccn3)c(C(F)(F)F)cc2=O)c1F] |
| 193 | 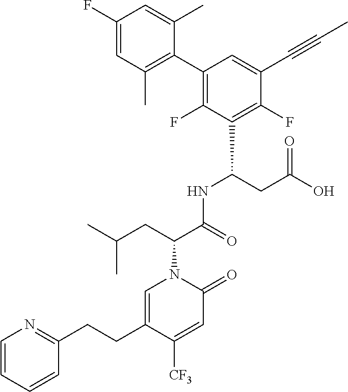 [SMILES: CC#Cc1cc(-c2c(C)cc(F)cc2C)c(F)c([C@H](CC(=O)O)NC(=O)[C@@H](CC(C)C)n2cc(CCc3ccccn3)c(C(F)(F)F)cc2=O)c1F] | 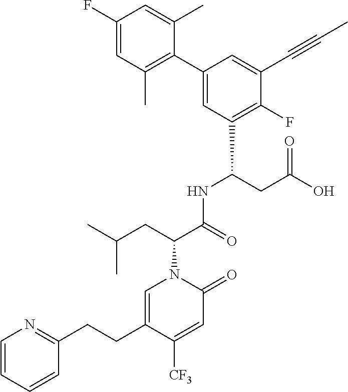 [SMILES: CC#Cc1cc(-c2c(C)cc(F)cc2C)cc([C@H](CC(=O)O)NC(=O)[C@@H](CC(C)C)n2cc(CCc3ccccn3)c(C(F)(F)F)cc2=O)c1F] |
| 194 | 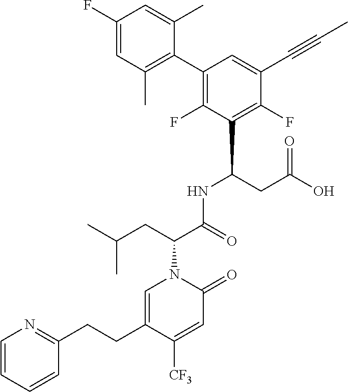 [SMILES: CC#Cc1cc(-c2c(C)cc(F)cc2C)c(F)c([C@@H](CC(=O)O)NC(=O)[C@@H](CC(C)C)n2cc(CCc3ccccn3)c(C(F)(F)F)cc2=O)c1F] | 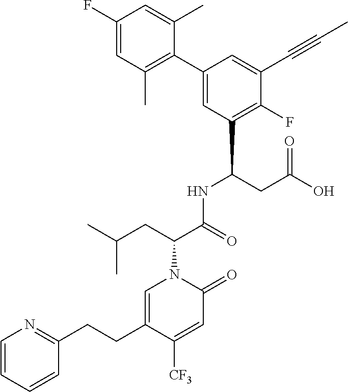 [SMILES: CC#Cc1cc(-c2c(C)cc(F)cc2C)cc([C@@H](CC(=O)O)NC(=O)[C@@H](CC(C)C)n2cc(CCc3ccccn3)c(C(F)(F)F)cc2=O)c1F] |
| 195 | 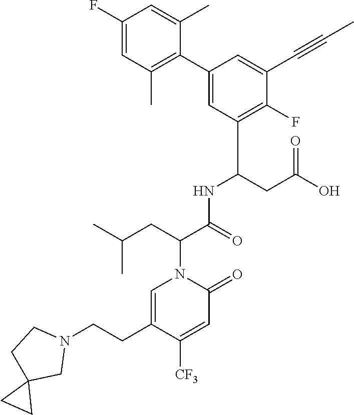 [SMILES: CC#Cc1cc(-c2c(C)cc(F)cc2C)cc(C(CC(=O)O)NC(=O)C(CC(C)C)n2cc(CCN3CCC4(CC4)C3)c(C(F)(F)F)cc2=O)c1F] | 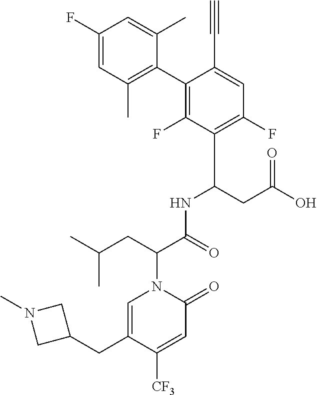 [SMILES: C#Cc1cc(F)c(C(CC(=O)O)NC(=O)C(CC(C)C)n2cc(CC3CN(C)C3)c(C(F)(F)F)cc2=O)c(F)c1-c1c(C)cc(F)cc1C] |
| 196 | 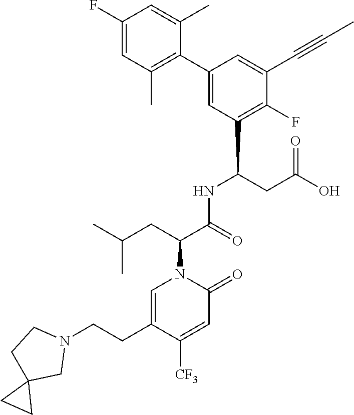 [SMILES: CC#Cc1cc(-c2c(C)cc(F)cc2C)cc([C@@H](CC(=O)O)NC(=O)[C@H](CC(C)C)n2cc(CCN3CCC4(CC4)C3)c(C(F)(F)F)cc2=O)c1F] | 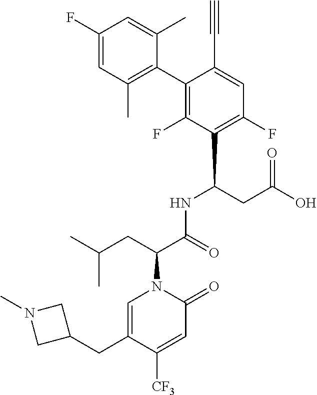 [SMILES: C#Cc1cc(F)c([C@@H](CC(=O)O)NC(=O)[C@H](CC(C)C)n2cc(CC3CN(C)C3)c(C(F)(F)F)cc2=O)c(F)c1-c1c(C)cc(F)cc1C] |
| 197 | 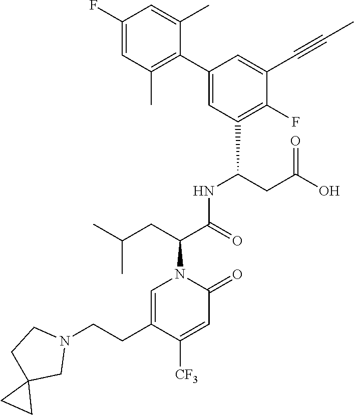 [SMILES: CC#Cc1cc(-c2c(C)cc(F)cc2C)cc([C@H](CC(=O)O)NC(=O)[C@H](CC(C)C)n2cc(CCN3CCC4(CC4)C3)c(C(F)(F)F)cc2=O)c1F] | 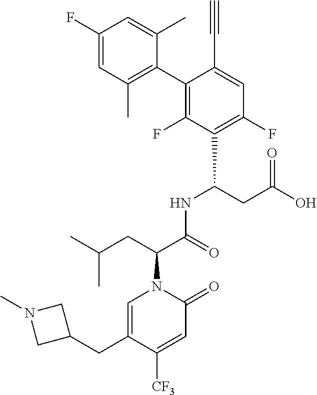 [SMILES: C#Cc1cc(F)c([C@H](CC(=O)O)NC(=O)[C@H](CC(C)C)n2cc(CC3CN(C)C3)c(C(F)(F)F)cc2=O)c(F)c1-c1c(C)cc(F)cc1C] |
| 198 | 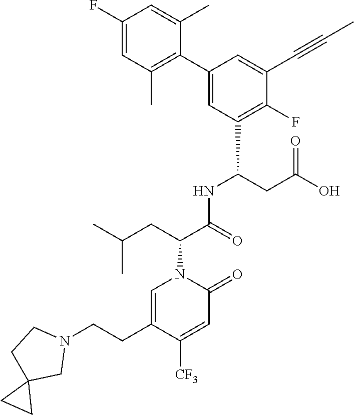 [SMILES: CC#Cc1cc(-c2c(C)cc(F)cc2C)cc([C@H](CC(=O)O)NC(=O)[C@@H](CC(C)C)n2cc(CCN3CCC4(CC4)C3)c(C(F)(F)F)cc2=O)c1F] | 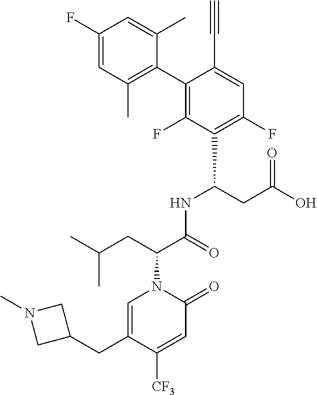 [SMILES: C#Cc1cc(F)c([C@H](CC(=O)O)NC(=O)[C@@H](CC(C)C)n2cc(CC3CN(C)C3)c(C(F)(F)F)cc2=O)c(F)c1-c1c(C)cc(F)cc1C] |
| 199 | 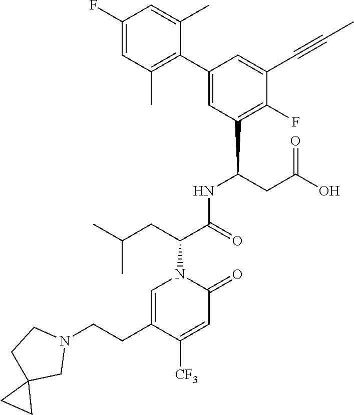 [SMILES: CC#Cc1cc(-c2c(C)cc(F)cc2C)cc([C@@H](CC(=O)O)NC(=O)[C@@H](CC(C)C)n2cc(CCN3CCC4(CC4)C3)c(C(F)(F)F)cc2=O)c1F] | 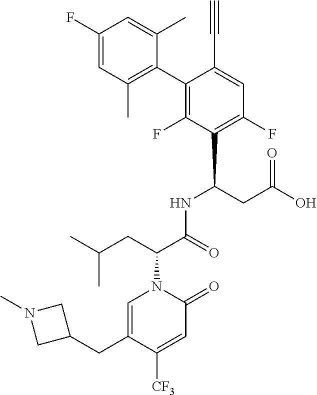 [SMILES: C#Cc1cc(F)c([C@@H](CC(=O)O)NC(=O)[C@@H](CC(C)C)n2cc(CC3CN(C)C3)c(C(F)(F)F)cc2=O)c(F)c1-c1c(C)cc(F)cc1C] |
| 200 | 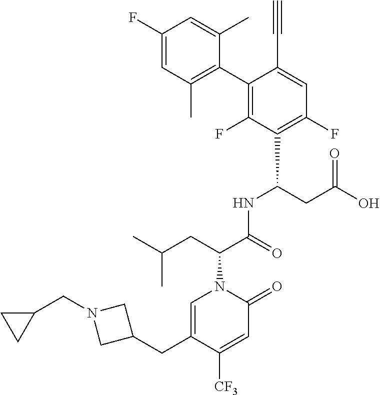 [SMILES: C#Cc1cc(F)c([C@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] | 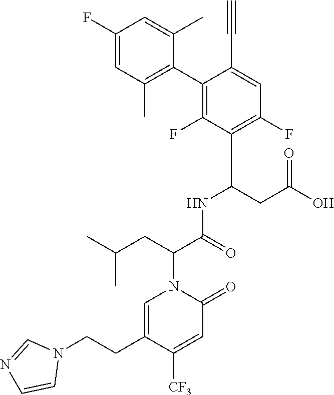 [SMILES: C#Cc1cc(F)c(C(CC(=O)O)NC(=O)C(CC(C)C)n2cc(CCn3ccnc3)c(C(F)(F)F)cc2=O)c(F)c1-c1c(C)cc(F)cc1C] |
| 201 | 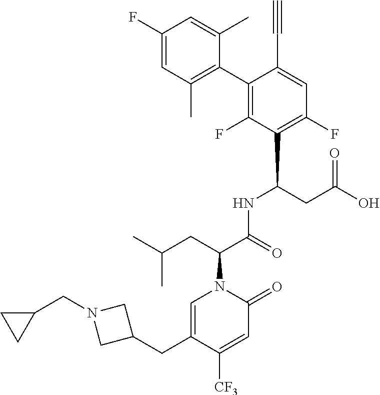 [SMILES: C#Cc1cc(F)c([C@@H](CC(=O)O)NC(=O)[C@H](CC(C)C)n2cc(CC3CN(CC4CC4)C3)c(C(F)(F)F)cc2=O)c(F)c1-c1c(C)cc(F)cc1C] | 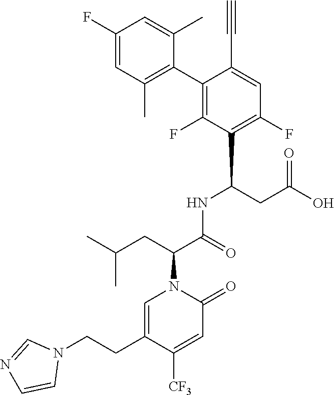 [SMILES: C#Cc1cc(F)c([C@@H](CC(=O)O)NC(=O)[C@H](CC(C)C)n2cc(CCn3ccnc3)c(C(F)(F)F)cc2=O)c(F)c1-c1c(C)cc(F)cc1C] |
| 202 | 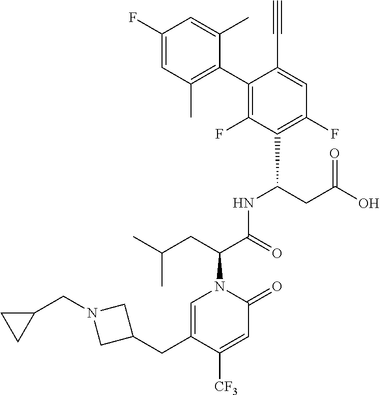 [SMILES: C#Cc1cc(F)c([C@H](CC(=O)O)NC(=O)[C@H](CC(C)C)n2cc(CC3CN(CC4CC4)C3)c(C(F)(F)F)cc2=O)c(F)c1-c1c(C)cc(F)cc1C] | 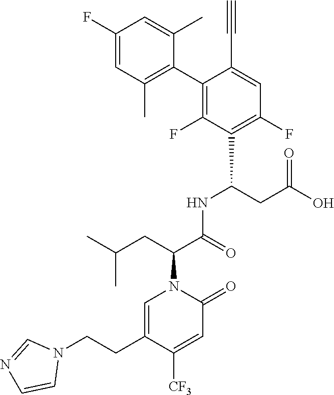 [SMILES: C#Cc1cc(F)c([C@H](CC(=O)O)NC(=O)[C@H](CC(C)C)n2cc(CCn3ccnc3)c(C(F)(F)F)cc2=O)c(F)c1-c1c(C)cc(F)cc1C] |
| 203 | 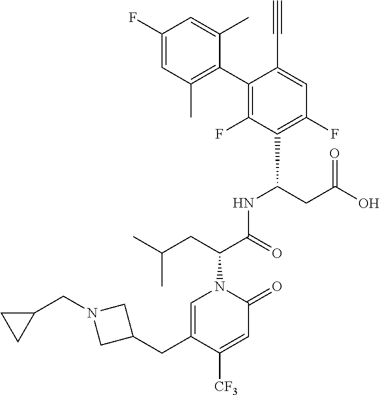 [SMILES: C#Cc1cc(F)c([C@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] | 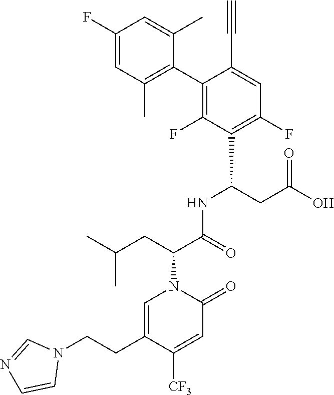 [SMILES: C#Cc1cc(F)c([C@H](CC(=O)O)NC(=O)[C@@H](CC(C)C)n2cc(CCn3ccnc3)c(C(F)(F)F)cc2=O)c(F)c1-c1c(C)cc(F)cc1C] |
| 204 | 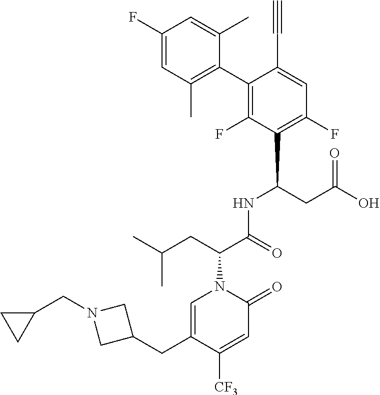 [SMILES: C#Cc1cc(F)c([C@@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] | 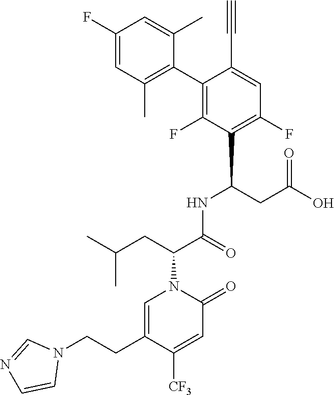 [SMILES: C#Cc1cc(F)c([C@@H](CC(=O)O)NC(=O)[C@@H](CC(C)C)n2cc(CCn3ccnc3)c(C(F)(F)F)cc2=O)c(F)c1-c1c(C)cc(F)cc1C] |
| 205 | 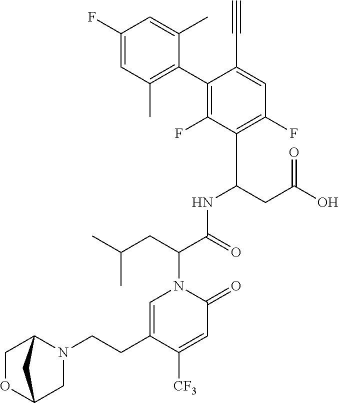 [SMILES: C#Cc1cc(F)c(C(CC(=O)O)NC(=O)C(CC(C)C)n2cc(CCN3C[C@H]4C[C@@H]3CO4)c(C(F)(F)F)cc2=O)c(F)c1-c1c(C)cc(F)cc1C] | 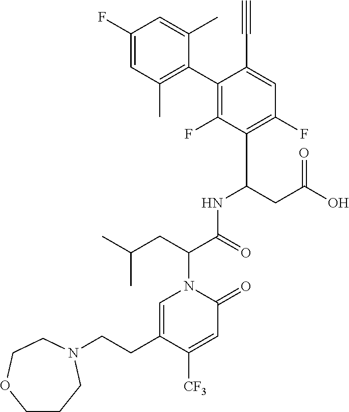 [SMILES: C#Cc1cc(F)c(C(CC(=O)O)NC(=O)C(CC(C)C)n2cc(CCN3CCCOCC3)c(C(F)(F)F)cc2=O)c(F)c1-c1c(C)cc(F)cc1C] |
| 206 | 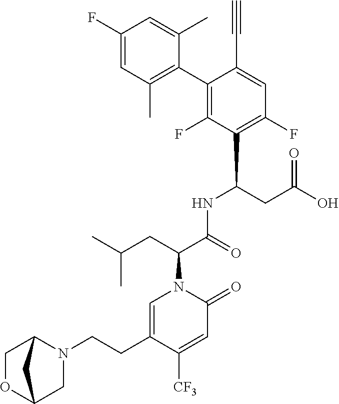 [SMILES: C#Cc1cc(F)c([C@@H](CC(=O)O)NC(=O)[C@H](CC(C)C)n2cc(CCN3C[C@H]4C[C@@H]3CO4)c(C(F)(F)F)cc2=O)c(F)c1-c1c(C)cc(F)cc1C] | 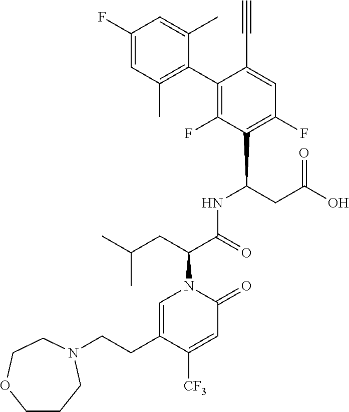 [SMILES: C#Cc1cc(F)c([C@@H](CC(=O)O)NC(=O)[C@H](CC(C)C)n2cc(CCN3CCCOCC3)c(C(F)(F)F)cc2=O)c(F)c1-c1c(C)cc(F)cc1C] |
| 207 | 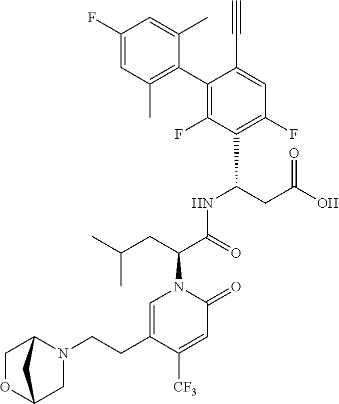 [SMILES: C#Cc1cc(F)c([C@H](CC(=O)O)NC(=O)[C@H](CC(C)C)n2cc(CCN3C[C@H]4C[C@@H]3CO4)c(C(F)(F)F)cc2=O)c(F)c1-c1c(C)cc(F)cc1C] | 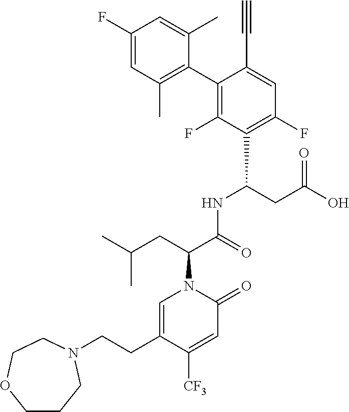 [SMILES: C#Cc1cc(F)c([C@H](CC(=O)O)NC(=O)[C@H](CC(C)C)n2cc(CCN3CCCOCC3)c(C(F)(F)F)cc2=O)c(F)c1-c1c(C)cc(F)cc1C] |
| 208 | 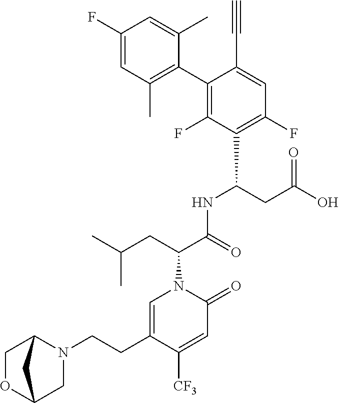 [SMILES: C#Cc1cc(F)c([C@H](CC(=O)O)NC(=O)[C@@H](CC(C)C)n2cc(CCN3C[C@H]4C[C@@H]3CO4)c(C(F)(F)F)cc2=O)c(F)c1-c1c(C)cc(F)cc1C] | 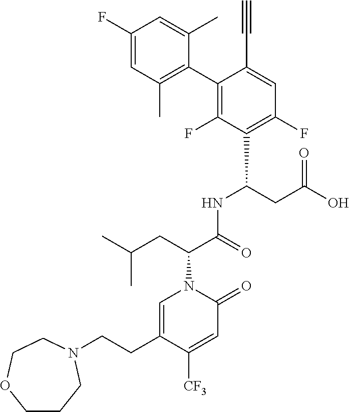 [SMILES: C#Cc1cc(F)c([C@H](CC(=O)O)NC(=O)[C@@H](CC(C)C)n2cc(CCN3CCCOCC3)c(C(F)(F)F)cc2=O)c(F)c1-c1c(C)cc(F)cc1C] |
| 209 | 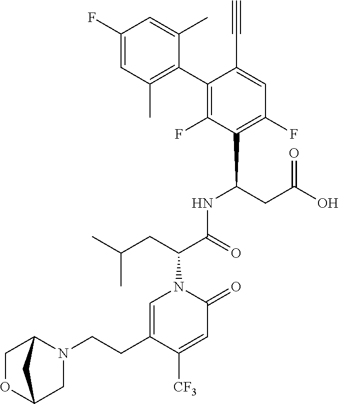 [SMILES: C#Cc1cc(F)c([C@@H](CC(=O)O)NC(=O)[C@@H](CC(C)C)n2cc(CCN3C[C@H]4C[C@@H]3CO4)c(C(F)(F)F)cc2=O)c(F)c1-c1c(C)cc(F)cc1C] | 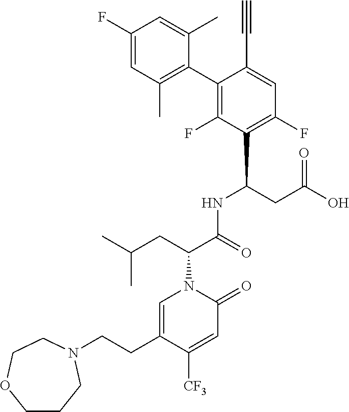 [SMILES: C#Cc1cc(F)c([C@@H](CC(=O)O)NC(=O)[C@@H](CC(C)C)n2cc(CCN3CCCOCC3)c(C(F)(F)F)cc2=O)c(F)c1-c1c(C)cc(F)cc1C] |
| 210 | 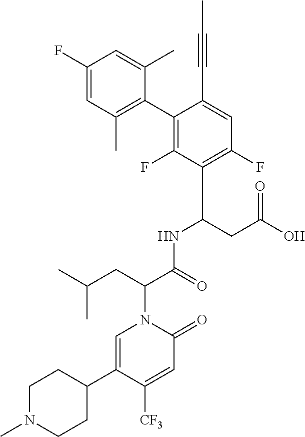 [SMILES: CC#Cc1cc(F)c(C(CC(=O)O)NC(=O)C(CC(C)C)n2cc(C3CCN(C)CC3)c(C(F)(F)F)cc2=O)c(F)c1-c1c(C)cc(F)cc1C] | 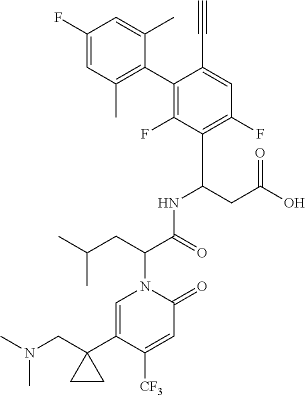 [SMILES: C#Cc1cc(F)c(C(CC(=O)O)NC(=O)C(CC(C)C)n2cc(C3(CN(C)C)CC3)c(C(F)(F)F)cc2=O)c(F)c1-c1c(C)cc(F)cc1C] |
| 211 | 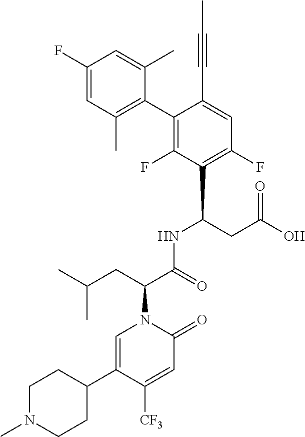 [SMILES: CC#Cc1cc(F)c([C@@H](CC(=O)O)NC(=O)[C@H](CC(C)C)n2cc(C3CCN(C)CC3)c(C(F)(F)F)cc2=O)c(F)c1-c1c(C)cc(F)cc1C] | 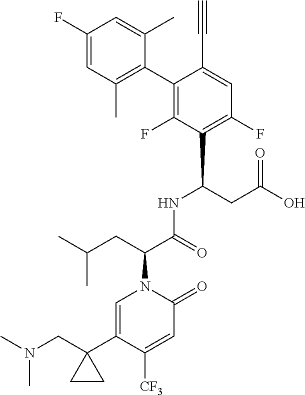 [SMILES: C#Cc1cc(F)c([C@@H](CC(=O)O)NC(=O)[C@H](CC(C)C)n2cc(C3(CN(C)C)CC3)c(C(F)(F)F)cc2=O)c(F)c1-c1c(C)cc(F)cc1C] |
| 212 | 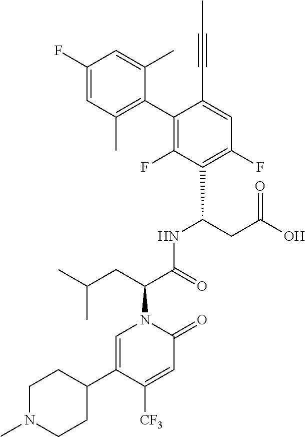 [SMILES: CC#Cc1cc(F)c([C@H](CC(=O)O)NC(=O)[C@H](CC(C)C)n2cc(C3CCN(C)CC3)c(C(F)(F)F)cc2=O)c(F)c1-c1c(C)cc(F)cc1C] | 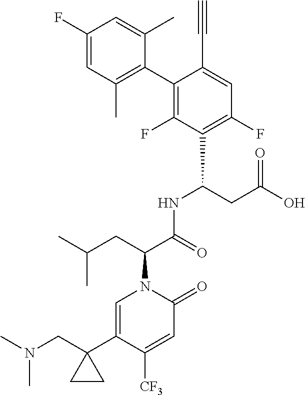 [SMILES: C#Cc1cc(F)c([C@H](CC(=O)O)NC(=O)[C@H](CC(C)C)n2cc(C3(CN(C)C)CC3)c(C(F)(F)F)cc2=O)c(F)c1-c1c(C)cc(F)cc1C] |
| 213 | 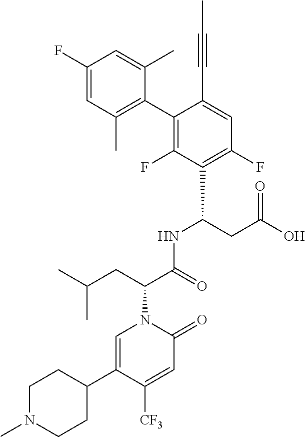 [SMILES: CC#Cc1cc(F)c([C@H](CC(=O)O)NC(=O)[C@@H](CC(C)C)n2cc(C3CCN(C)CC3)c(C(F)(F)F)cc2=O)c(F)c1-c1c(C)cc(F)cc1C] | 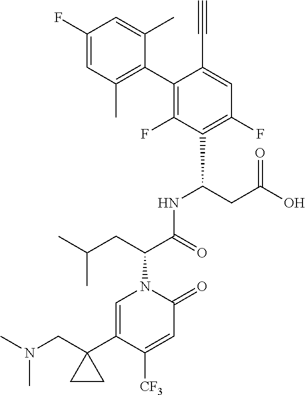 [SMILES: C#Cc1cc(F)c([C@H](CC(=O)O)NC(=O)[C@@H](CC(C)C)n2cc(C3(CN(C)C)CC3)c(C(F)(F)F)cc2=O)c(F)c1-c1c(C)cc(F)cc1C] |
| 214 | 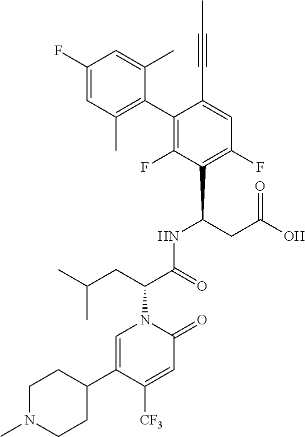 [SMILES: CC#Cc1cc(F)c([C@@H](CC(=O)O)NC(=O)[C@@H](CC(C)C)n2cc(C3CCN(C)CC3)c(C(F)(F)F)cc2=O)c(F)c1-c1c(C)cc(F)cc1C] | 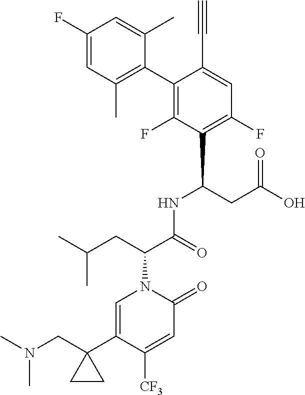 [SMILES: C#Cc1cc(F)c([C@@H](CC(=O)O)NC(=O)[C@@H](CC(C)C)n2cc(C3(CN(C)C)CC3)c(C(F)(F)F)cc2=O)c(F)c1-c1c(C)cc(F)cc1C] |
| 215 | 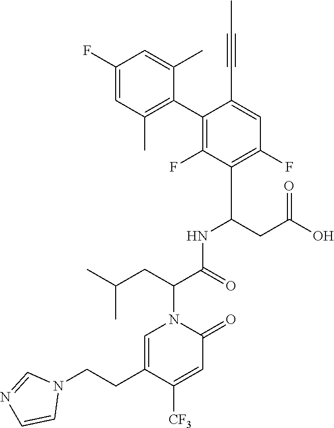 [SMILES: CC#Cc1cc(F)c(C(CC(=O)O)NC(=O)C(CC(C)C)n2cc(CCn3ccnc3)c(C(F)(F)F)cc2=O)c(F)c1-c1c(C)cc(F)cc1C] | 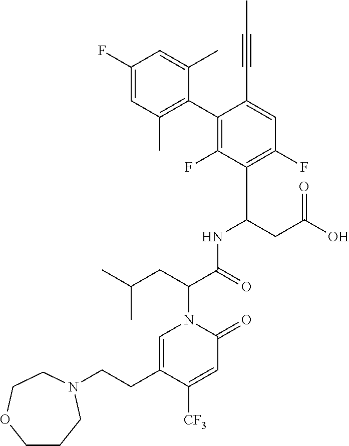 [SMILES: CC#Cc1cc(F)c(C(CC(=O)O)NC(=O)C(CC(C)C)n2cc(CCN3CCCOCC3)c(C(F)(F)F)cc2=O)c(F)c1-c1c(C)cc(F)cc1C] |
| 216 | 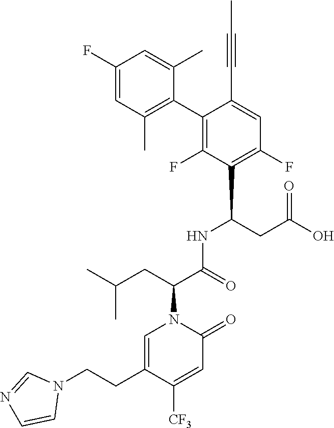 [SMILES: CC#Cc1cc(F)c([C@@H](CC(=O)O)NC(=O)[C@H](CC(C)C)n2cc(CCn3ccnc3)c(C(F)(F)F)cc2=O)c(F)c1-c1c(C)cc(F)cc1C] | 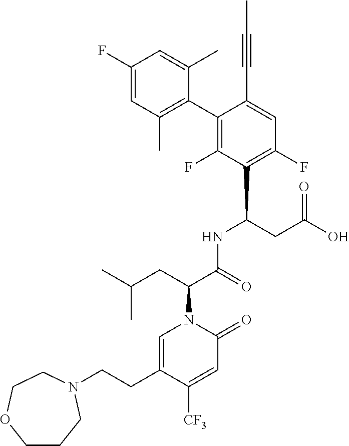 [SMILES: CC#Cc1cc(F)c([C@@H](CC(=O)O)NC(=O)[C@H](CC(C)C)n2cc(CCN3CCCOCC3)c(C(F)(F)F)cc2=O)c(F)c1-c1c(C)cc(F)cc1C] |
| 217 | 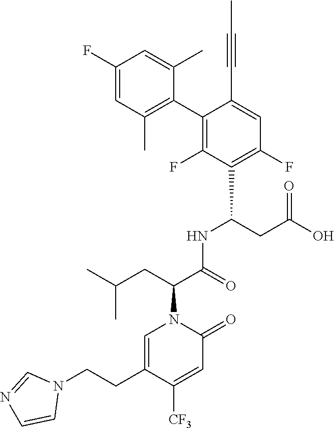 [SMILES: CC#Cc1cc(F)c([C@H](CC(=O)O)NC(=O)[C@H](CC(C)C)n2cc(CCn3ccnc3)c(C(F)(F)F)cc2=O)c(F)c1-c1c(C)cc(F)cc1C] | 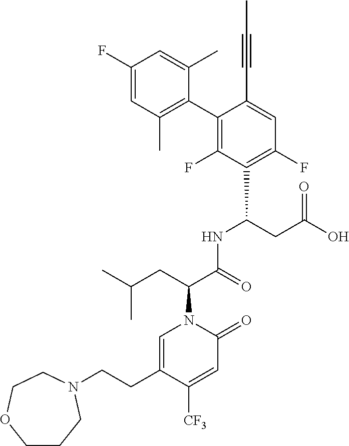 [SMILES: CC#Cc1cc(F)c([C@H](CC(=O)O)NC(=O)[C@H](CC(C)C)n2cc(CCN3CCCOCC3)c(C(F)(F)F)cc2=O)c(F)c1-c1c(C)cc(F)cc1C] |
| 218 | 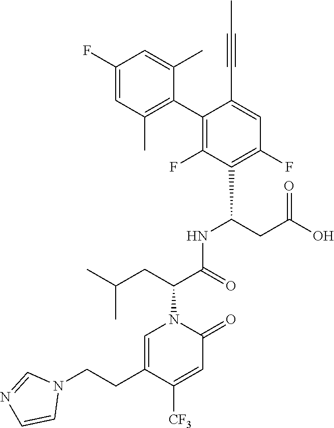 [SMILES: CC#Cc1cc(F)c([C@H](CC(=O)O)NC(=O)[C@@H](CC(C)C)n2cc(CCn3ccnc3)c(C(F)(F)F)cc2=O)c(F)c1-c1c(C)cc(F)cc1C] | 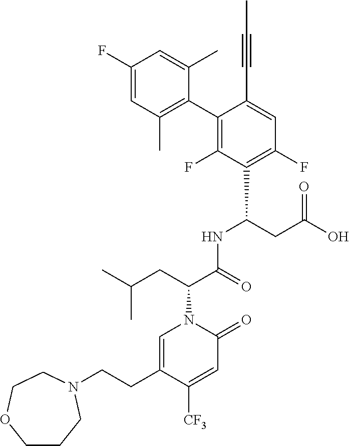 [SMILES: CC#Cc1cc(F)c([C@H](CC(=O)O)NC(=O)[C@@H](CC(C)C)n2cc(CCN3CCCOCC3)c(C(F)(F)F)cc2=O)c(F)c1-c1c(C)cc(F)cc1C] |
| 219 | 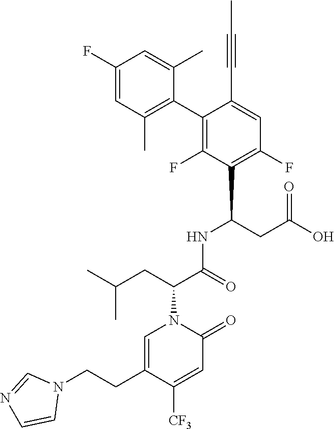 [SMILES: CC#Cc1cc(F)c([C@@H](CC(=O)O)NC(=O)[C@@H](CC(C)C)n2cc(CCn3ccnc3)c(C(F)(F)F)cc2=O)c(F)c1-c1c(C)cc(F)cc1C] | 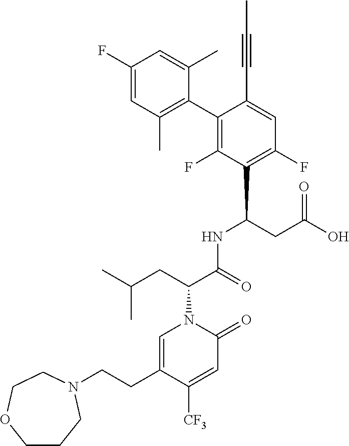 [SMILES: CC#Cc1cc(F)c([C@@H](CC(=O)O)NC(=O)[C@@H](CC(C)C)n2cc(CCN3CCCOCC3)c(C(F)(F)F)cc2=O)c(F)c1-c1c(C)cc(F)cc1C] |
| 220 | 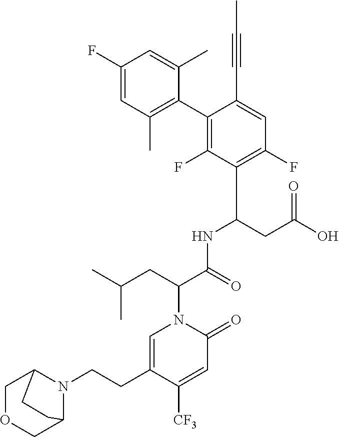 [SMILES: CC#Cc1cc(F)c(C(CC(=O)O)NC(=O)C(CC(C)C)n2cc(CCN3C4CCC3COC4)c(C(F)(F)F)cc2=O)c(F)c1-c1c(C)cc(F)cc1C] | 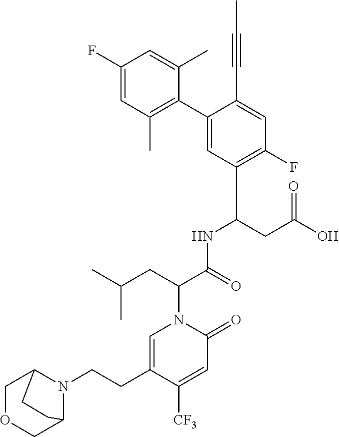 [SMILES: CC#Cc1cc(F)c(C(CC(=O)O)NC(=O)C(CC(C)C)n2cc(CCN3C4CCC3COC4)c(C(F)(F)F)cc2=O)cc1-c1c(C)cc(F)cc1C] |
| 221 | 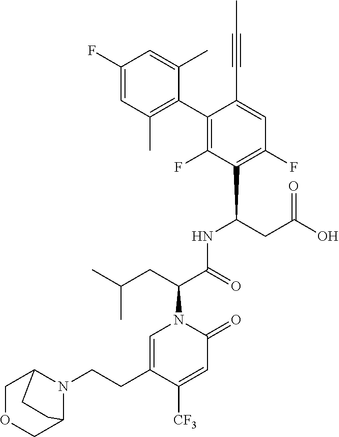 [SMILES: CC#Cc1cc(F)c([C@@H](CC(=O)O)NC(=O)[C@H](CC(C)C)n2cc(CCN3C4CCC3COC4)c(C(F)(F)F)cc2=O)c(F)c1-c1c(C)cc(F)cc1C] | 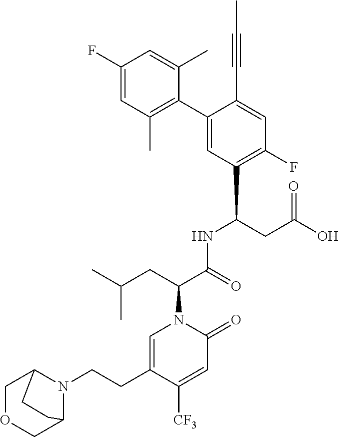 [SMILES: CC#Cc1cc(F)c([C@@H](CC(=O)O)NC(=O)[C@H](CC(C)C)n2cc(CCN3C4CCC3COC4)c(C(F)(F)F)cc2=O)cc1-c1c(C)cc(F)cc1C] |
| 222 | 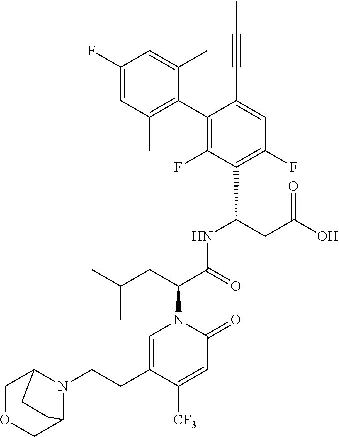 [SMILES: CC#Cc1cc(F)c([C@H](CC(=O)O)NC(=O)[C@H](CC(C)C)n2cc(CCN3C4CCC3COC4)c(C(F)(F)F)cc2=O)c(F)c1-c1c(C)cc(F)cc1C] | 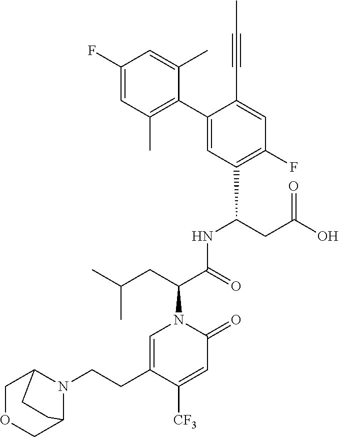 [SMILES: CC#Cc1cc(F)c([C@H](CC(=O)O)NC(=O)[C@H](CC(C)C)n2cc(CCN3C4CCC3COC4)c(C(F)(F)F)cc2=O)cc1-c1c(C)cc(F)cc1C] |
| 223 | 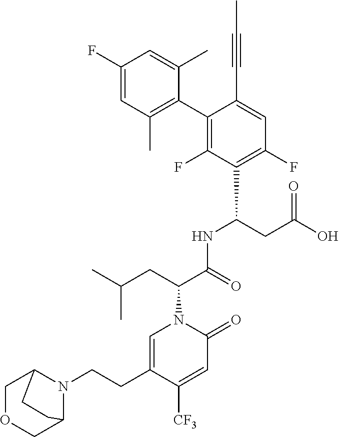 [SMILES: CC#Cc1cc(F)c([C@H](CC(=O)O)NC(=O)[C@@H](CC(C)C)n2cc(CCN3C4CCC3COC4)c(C(F)(F)F)cc2=O)c(F)c1-c1c(C)cc(F)cc1C] | 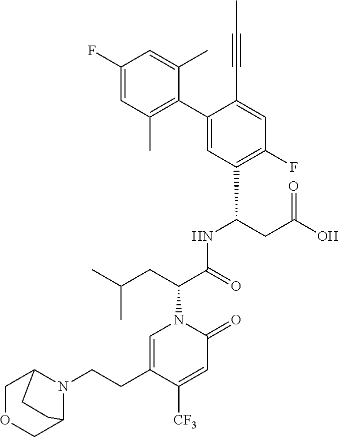 [SMILES: CC#Cc1cc(F)c([C@H](CC(=O)O)NC(=O)[C@@H](CC(C)C)n2cc(CCN3C4CCC3COC4)c(C(F)(F)F)cc2=O)cc1-c1c(C)cc(F)cc1C] |
| 224 | 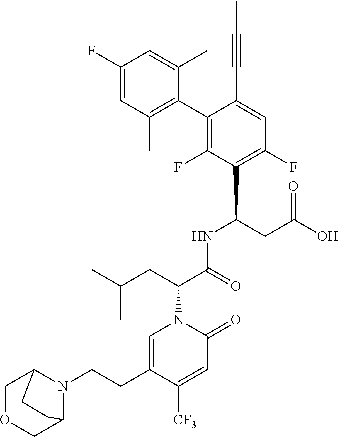 [SMILES: CC#Cc1cc(F)c([C@@H](CC(=O)O)NC(=O)[C@@H](CC(C)C)n2cc(CCN3C4CCC3COC4)c(C(F)(F)F)cc2=O)c(F)c1-c1c(C)cc(F)cc1C] | 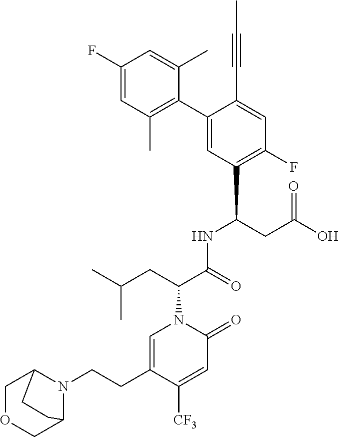 [SMILES: CC#Cc1cc(F)c([C@@H](CC(=O)O)NC(=O)[C@@H](CC(C)C)n2cc(CCN3C4CCC3COC4)c(C(F)(F)F)cc2=O)cc1-c1c(C)cc(F)cc1C] |
| 225 | 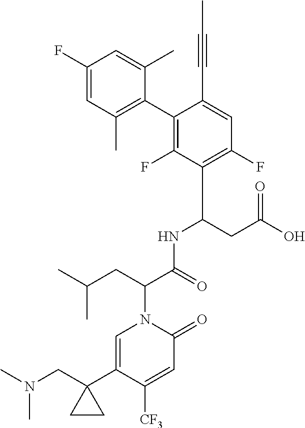 [SMILES: CC#Cc1cc(F)c(C(CC(=O)O)NC(=O)C(CC(C)C)n2cc(C3(CN(C)C)CC3)c(C(F)(F)F)cc2=O)c(F)c1-c1c(C)cc(F)cc1C] | 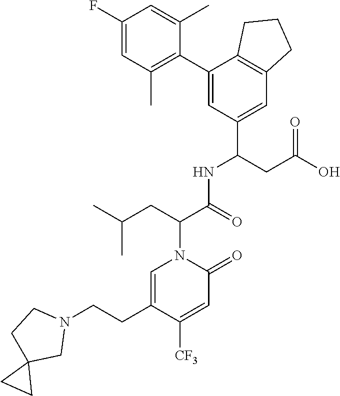 [SMILES: Cc1cc(F)cc(C)c1-c1cc(C(CC(=O)O)NC(=O)C(CC(C)C)n2cc(CCN3CCC4(CC4)C3)c(C(F)(F)F)cc2=O)cc2c1CCC2] |
| 226 | 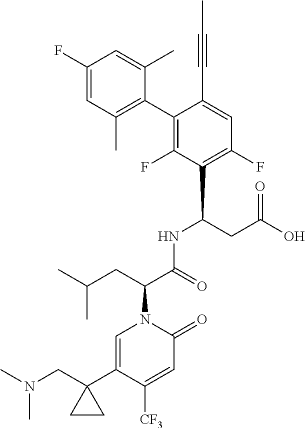 [SMILES: CC#Cc1cc(F)c([C@@H](CC(=O)O)NC(=O)[C@H](CC(C)C)n2cc(C3(CN(C)C)CC3)c(C(F)(F)F)cc2=O)c(F)c1-c1c(C)cc(F)cc1C] | 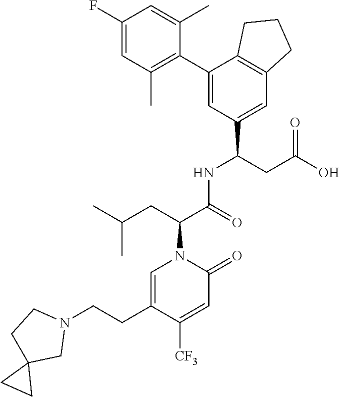 [SMILES: Cc1cc(F)cc(C)c1-c1cc([C@@H](CC(=O)O)NC(=O)[C@H](CC(C)C)n2cc(CCN3CCC4(CC4)C3)c(C(F)(F)F)cc2=O)cc2c1CCC2] |
| 227 | 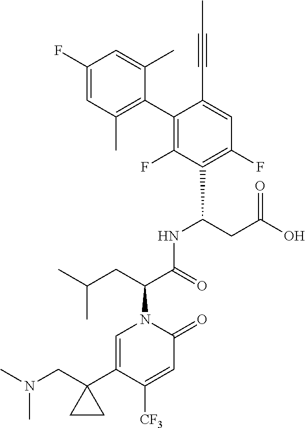 [SMILES: CC#Cc1cc(F)c([C@H](CC(=O)O)NC(=O)[C@H](CC(C)C)n2cc(C3(CN(C)C)CC3)c(C(F)(F)F)cc2=O)c(F)c1-c1c(C)cc(F)cc1C] | 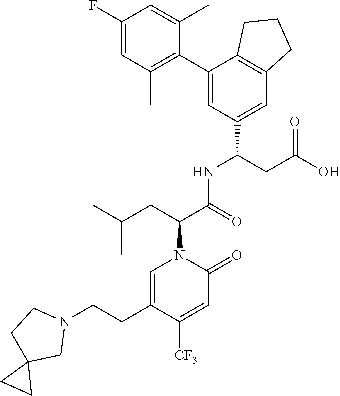 [SMILES: Cc1cc(F)cc(C)c1-c1cc([C@H](CC(=O)O)NC(=O)[C@H](CC(C)C)n2cc(CCN3CCC4(CC4)C3)c(C(F)(F)F)cc2=O)cc2c1CCC2] |
| 228 | 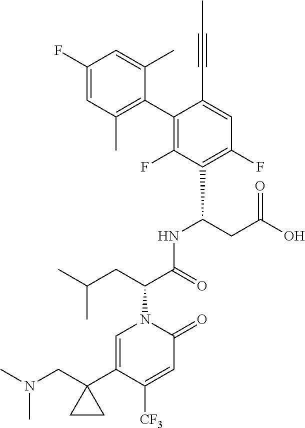 [SMILES: CC#Cc1cc(F)c([C@H](CC(=O)O)NC(=O)[C@@H](CC(C)C)n2cc(C3(CN(C)C)CC3)c(C(F)(F)F)cc2=O)c(F)c1-c1c(C)cc(F)cc1C] | 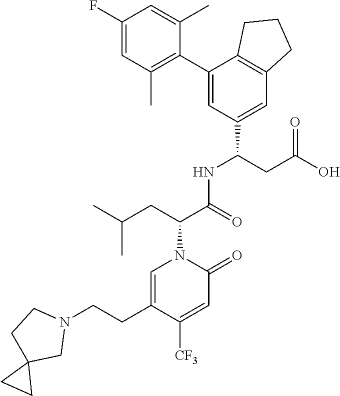 [SMILES: Cc1cc(F)cc(C)c1-c1cc([C@H](CC(=O)O)NC(=O)[C@@H](CC(C)C)n2cc(CCN3CCC4(CC4)C3)c(C(F)(F)F)cc2=O)cc2c1CCC2] |
| 229 | 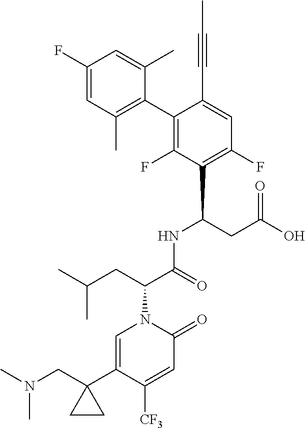 [SMILES: CC#Cc1cc(F)c([C@@H](CC(=O)O)NC(=O)[C@@H](CC(C)C)n2cc(C3(CN(C)C)CC3)c(C(F)(F)F)cc2=O)c(F)c1-c1c(C)cc(F)cc1C] | 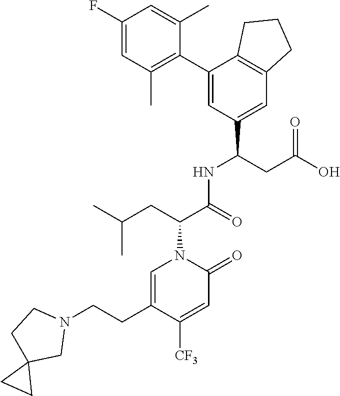 [SMILES: Cc1cc(F)cc(C)c1-c1cc([C@@H](CC(=O)O)NC(=O)[C@@H](CC(C)C)n2cc(CCN3CCC4(CC4)C3)c(C(F)(F)F)cc2=O)cc2c1CCC2] |
| 230 | 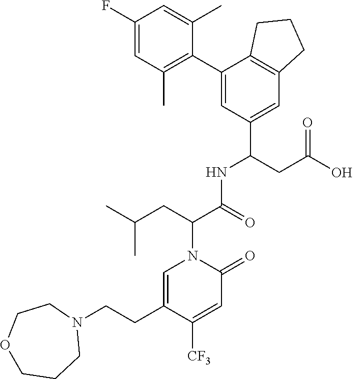 [SMILES: Cc1cc(F)cc(C)c1-c1cc(C(CC(=O)O)NC(=O)C(CC(C)C)n2cc(CCN3CCCOCC3)c(C(F)(F)F)cc2=O)cc2c1CCC2] | 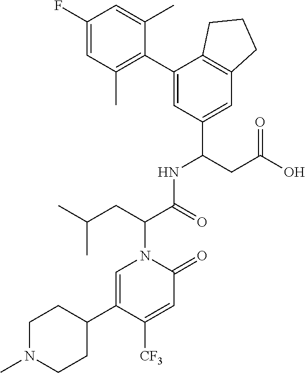 [SMILES: Cc1cc(F)cc(C)c1-c1cc(C(CC(=O)O)NC(=O)C(CC(C)C)n2cc(C3CCN(C)CC3)c(C(F)(F)F)cc2=O)cc2c1CCC2] |
| 231 | 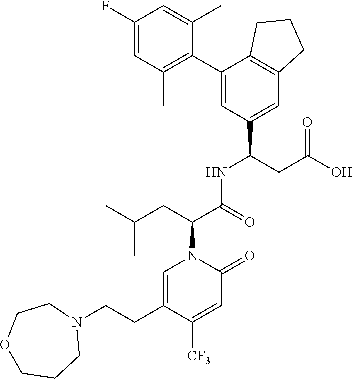 [SMILES: Cc1cc(F)cc(C)c1-c1cc([C@@H](CC(=O)O)NC(=O)[C@H](CC(C)C)n2cc(CCN3CCCOCC3)c(C(F)(F)F)cc2=O)cc2c1CCC2] | 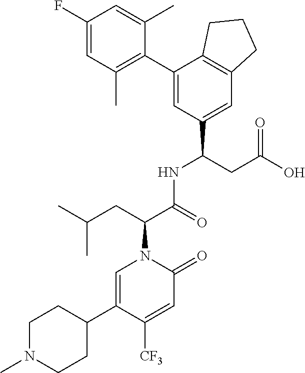 [SMILES: Cc1cc(F)cc(C)c1-c1cc([C@@H](CC(=O)O)NC(=O)[C@H](CC(C)C)n2cc(C3CCN(C)CC3)c(C(F)(F)F)cc2=O)cc2c1CCC2] |
| 232 | 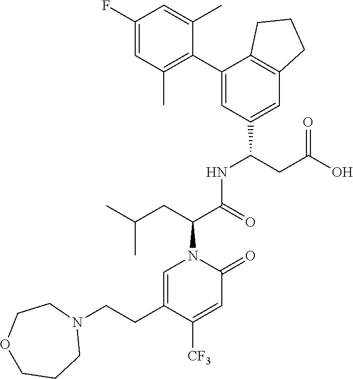 [SMILES: Cc1cc(F)cc(C)c1-c1cc([C@H](CC(=O)O)NC(=O)[C@H](CC(C)C)n2cc(CCN3CCCOCC3)c(C(F)(F)F)cc2=O)cc2c1CCC2] | 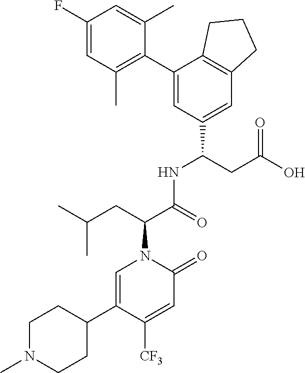 [SMILES: Cc1cc(F)cc(C)c1-c1cc([C@H](CC(=O)O)NC(=O)[C@H](CC(C)C)n2cc(C3CCN(C)CC3)c(C(F)(F)F)cc2=O)cc2c1CCC2] |
| 233 | 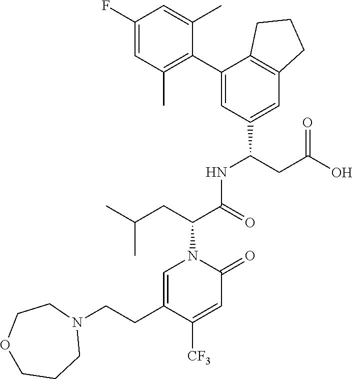 [SMILES: Cc1cc(F)cc(C)c1-c1cc([C@H](CC(=O)O)NC(=O)[C@@H](CC(C)C)n2cc(CCN3CCCOCC3)c(C(F)(F)F)cc2=O)cc2c1CCC2] | 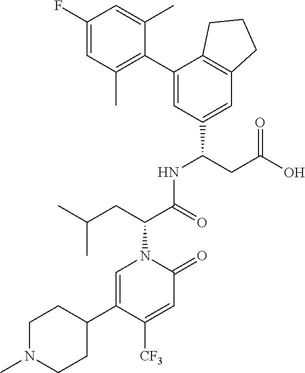 [SMILES: Cc1cc(F)cc(C)c1-c1cc([C@H](CC(=O)O)NC(=O)[C@@H](CC(C)C)n2cc(C3CCN(C)CC3)c(C(F)(F)F)cc2=O)cc2c1CCC2] |
| 234 | 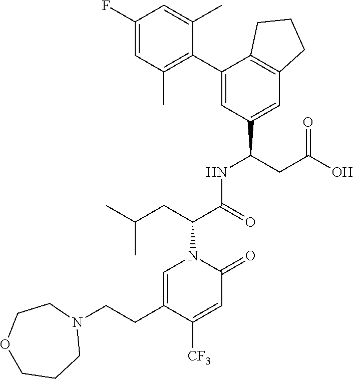 [SMILES: Cc1cc(F)cc(C)c1-c1cc([C@@H](CC(=O)O)NC(=O)[C@@H](CC(C)C)n2cc(CCN3CCCOCC3)c(C(F)(F)F)cc2=O)cc2c1CCC2] | 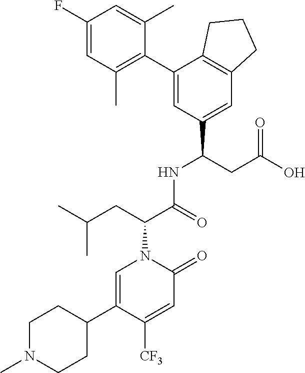 [SMILES: Cc1cc(F)cc(C)c1-c1cc([C@@H](CC(=O)O)NC(=O)[C@@H](CC(C)C)n2cc(C3CCN(C)CC3)c(C(F)(F)F)cc2=O)cc2c1CCC2] |
| 235 | 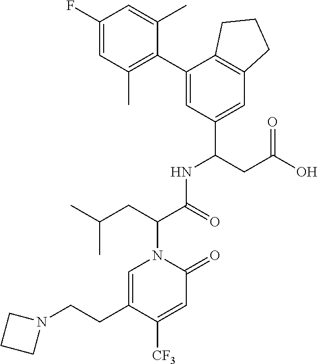 [SMILES: Cc1cc(F)cc(C)c1-c1cc(C(CC(=O)O)NC(=O)C(CC(C)C)n2cc(CCN3CCC3)c(C(F)(F)F)cc2=O)cc2c1CCC2] | 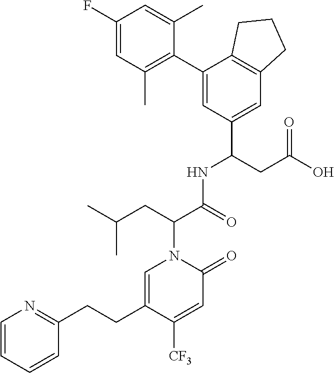 [SMILES: Cc1cc(F)cc(C)c1-c1cc(C(CC(=O)O)NC(=O)C(CC(C)C)n2cc(CCc3ccccn3)c(C(F)(F)F)cc2=O)cc2c1CCC2] |
| 236 | 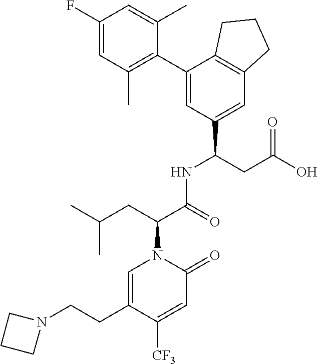 [SMILES: Cc1cc(F)cc(C)c1-c1cc([C@@H](CC(=O)O)NC(=O)[C@H](CC(C)C)n2cc(CCN3CCC3)c(C(F)(F)F)cc2=O)cc2c1CCC2] | 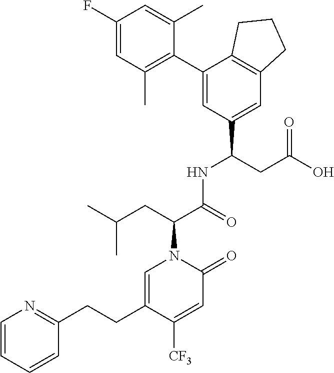 [SMILES: Cc1cc(F)cc(C)c1-c1cc([C@@H](CC(=O)O)NC(=O)[C@H](CC(C)C)n2cc(CCc3ccccn3)c(C(F)(F)F)cc2=O)cc2c1CCC2] |
| 237 | 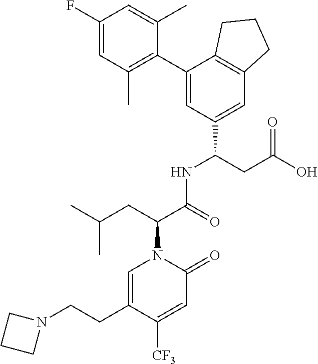 [SMILES: Cc1cc(F)cc(C)c1-c1cc([C@H](CC(=O)O)NC(=O)[C@H](CC(C)C)n2cc(CCN3CCC3)c(C(F)(F)F)cc2=O)cc2c1CCC2] | 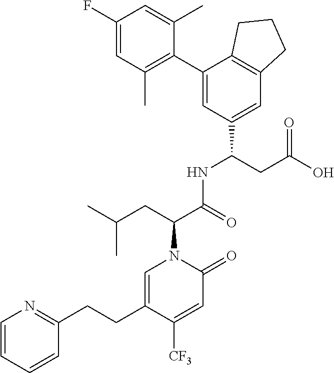 [SMILES: Cc1cc(F)cc(C)c1-c1cc([C@H](CC(=O)O)NC(=O)[C@H](CC(C)C)n2cc(CCc3ccccn3)c(C(F)(F)F)cc2=O)cc2c1CCC2] |
| 238 | 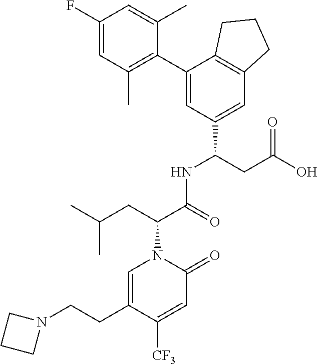 [SMILES: Cc1cc(F)cc(C)c1-c1cc([C@H](CC(=O)O)NC(=O)[C@@H](CC(C)C)n2cc(CCN3CCC3)c(C(F)(F)F)cc2=O)cc2c1CCC2] | 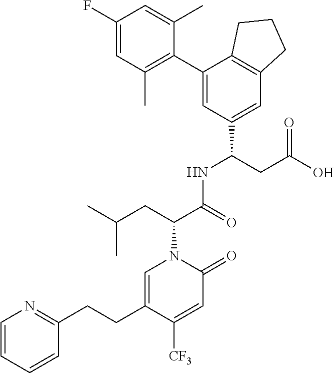 [SMILES: Cc1cc(F)cc(C)c1-c1cc([C@H](CC(=O)O)NC(=O)[C@@H](CC(C)C)n2cc(CCc3ccccn3)c(C(F)(F)F)cc2=O)cc2c1CCC2] |
| 239 | 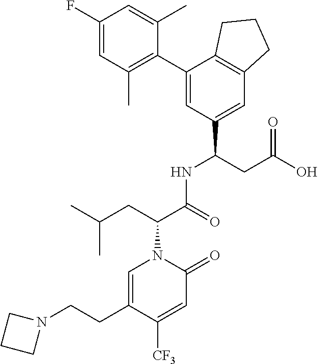 [SMILES: Cc1cc(F)cc(C)c1-c1cc([C@@H](CC(=O)O)NC(=O)[C@@H](CC(C)C)n2cc(CCN3CCC3)c(C(F)(F)F)cc2=O)cc2c1CCC2] | 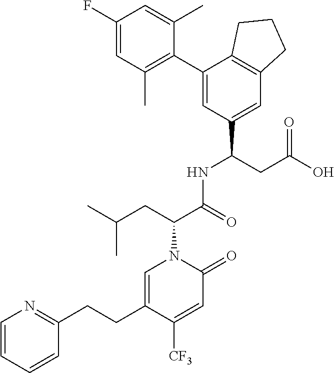 [SMILES: Cc1cc(F)cc(C)c1-c1cc([C@@H](CC(=O)O)NC(=O)[C@@H](CC(C)C)n2cc(CCc3ccccn3)c(C(F)(F)F)cc2=O)cc2c1CCC2] |
| 240 | 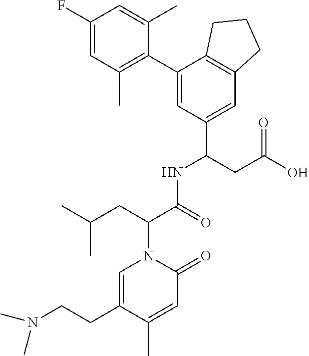 [SMILES: Cc1cc(=O)n(C(CC(C)C)C(=O)NC(CC(=O)O)c2cc3c(c(-c4c(C)cc(F)cc4C)c2)CCC3)cc1CCN(C)C] | 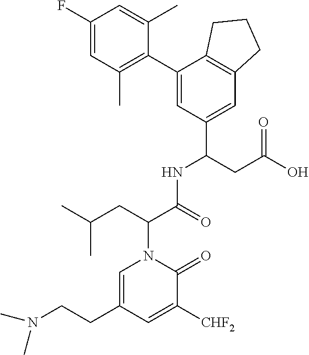 [SMILES: Cc1cc(F)cc(C)c1-c1cc(C(CC(=O)O)NC(=O)C(CC(C)C)n2cc(CCN(C)C)cc(C(F)F)c2=O)cc2c1CCC2] |
| 241 | 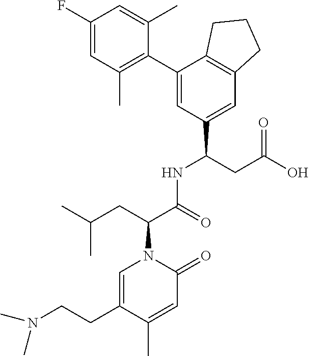 [SMILES: Cc1cc(=O)n([C@@H](CC(C)C)C(=O)N[C@H](CC(=O)O)c2cc3c(c(-c4c(C)cc(F)cc4C)c2)CCC3)cc1CCN(C)C] | 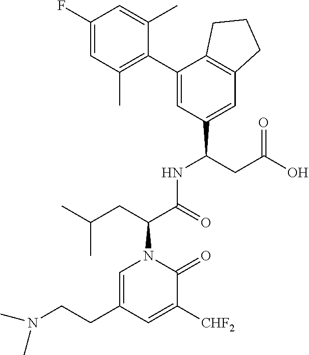 [SMILES: Cc1cc(F)cc(C)c1-c1cc([C@@H](CC(=O)O)NC(=O)[C@H](CC(C)C)n2cc(CCN(C)C)cc(C(F)F)c2=O)cc2c1CCC2] |
| 242 | 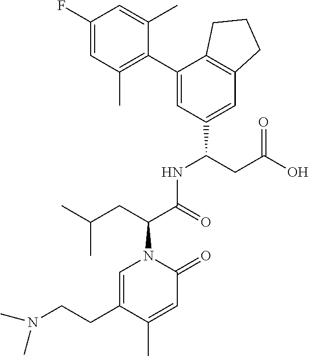 [SMILES: Cc1cc(=O)n([C@@H](CC(C)C)C(=O)N[C@@H](CC(=O)O)c2cc3c(c(-c4c(C)cc(F)cc4C)c2)CCC3)cc1CCN(C)C] | 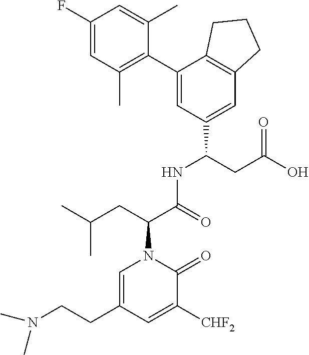 [SMILES: Cc1cc(F)cc(C)c1-c1cc([C@H](CC(=O)O)NC(=O)[C@H](CC(C)C)n2cc(CCN(C)C)cc(C(F)F)c2=O)cc2c1CCC2] |
| 243 | 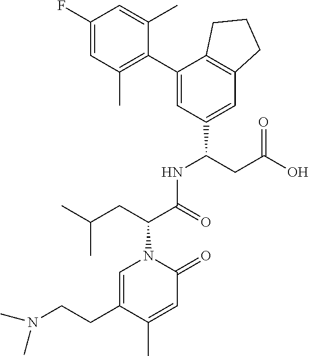 [SMILES: Cc1cc(=O)n([C@H](CC(C)C)C(=O)N[C@@H](CC(=O)O)c2cc3c(c(-c4c(C)cc(F)cc4C)c2)CCC3)cc1CCN(C)C] | 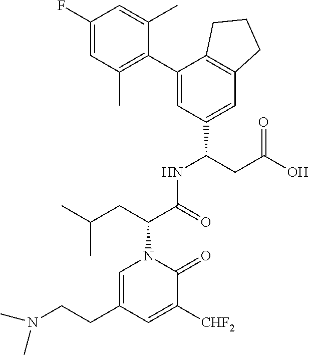 [SMILES: Cc1cc(F)cc(C)c1-c1cc([C@H](CC(=O)O)NC(=O)[C@@H](CC(C)C)n2cc(CCN(C)C)cc(C(F)F)c2=O)cc2c1CCC2] |
| 244 | 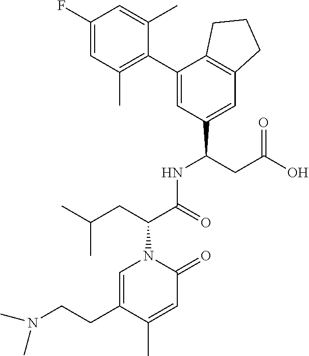 [SMILES: Cc1cc(=O)n([C@H](CC(C)C)C(=O)N[C@H](CC(=O)O)c2cc3c(c(-c4c(C)cc(F)cc4C)c2)CCC3)cc1CCN(C)C] | 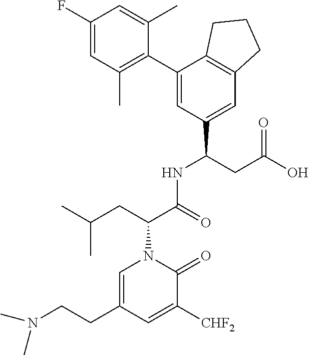 [SMILES: Cc1cc(F)cc(C)c1-c1cc([C@@H](CC(=O)O)NC(=O)[C@@H](CC(C)C)n2cc(CCN(C)C)cc(C(F)F)c2=O)cc2c1CCC2] |
| 245 | 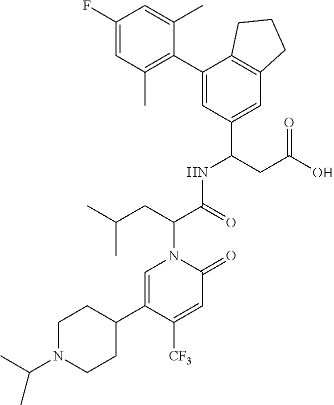 [SMILES: Cc1cc(F)cc(C)c1-c1cc(C(CC(=O)O)NC(=O)C(CC(C)C)n2cc(C3CCN(C(C)C)CC3)c(C(F)(F)F)cc2=O)cc2c1CCC2] | 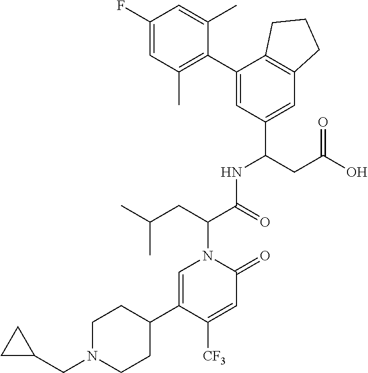 [SMILES: Cc1cc(F)cc(C)c1-c1cc(C(CC(=O)O)NC(=O)C(CC(C)C)n2cc(C3CCN(CC4CC4)CC3)c(C(F)(F)F)cc2=O)cc2c1CCC2] |
| 246 | 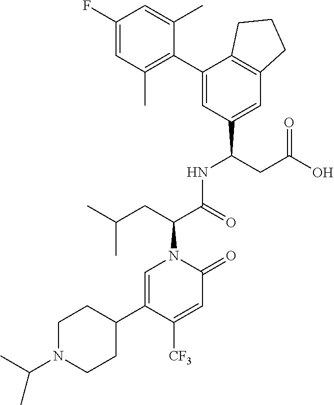 [SMILES: Cc1cc(F)cc(C)c1-c1cc([C@@H](CC(=O)O)NC(=O)[C@H](CC(C)C)n2cc(C3CCN(C(C)C)CC3)c(C(F)(F)F)cc2=O)cc2c1CCC2] | 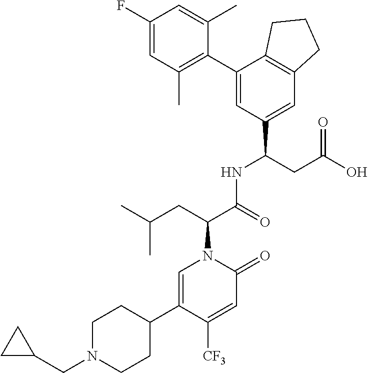 [SMILES: Cc1cc(F)cc(C)c1-c1cc([C@@H](CC(=O)O)NC(=O)[C@H](CC(C)C)n2cc(C3CCN(CC4CC4)CC3)c(C(F)(F)F)cc2=O)cc2c1CCC2] |
| 247 | 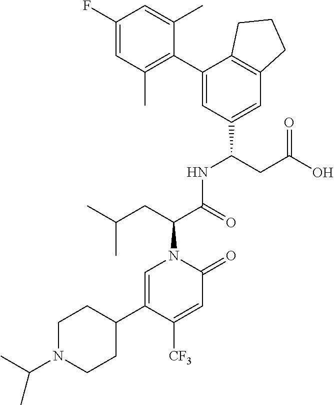 [SMILES: Cc1cc(F)cc(C)c1-c1cc([C@H](CC(=O)O)NC(=O)[C@H](CC(C)C)n2cc(C3CCN(C(C)C)CC3)c(C(F)(F)F)cc2=O)cc2c1CCC2] | 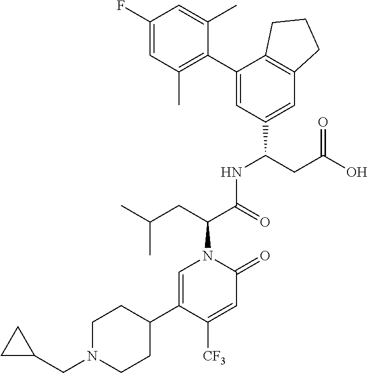 [SMILES: Cc1cc(F)cc(C)c1-c1cc([C@H](CC(=O)O)NC(=O)[C@H](CC(C)C)n2cc(C3CCN(CC4CC4)CC3)c(C(F)(F)F)cc2=O)cc2c1CCC2] |
| 248 | 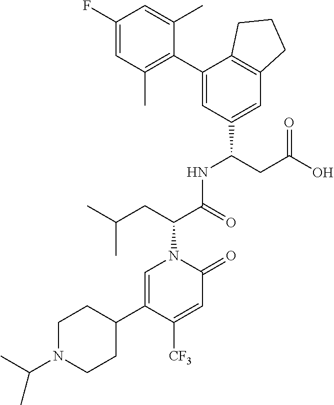 [SMILES: Cc1cc(F)cc(C)c1-c1cc([C@H](CC(=O)O)NC(=O)[C@@H](CC(C)C)n2cc(C3CCN(C(C)C)CC3)c(C(F)(F)F)cc2=O)cc2c1CCC2] | 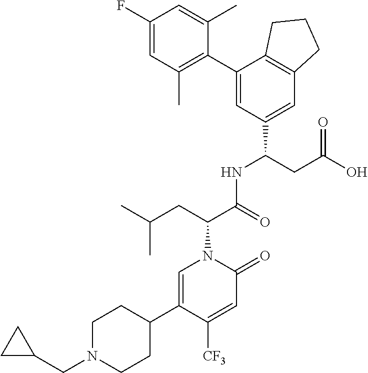 [SMILES: Cc1cc(F)cc(C)c1-c1cc([C@H](CC(=O)O)NC(=O)[C@@H](CC(C)C)n2cc(C3CCN(CC4CC4)CC3)c(C(F)(F)F)cc2=O)cc2c1CCC2] |
| 249 | 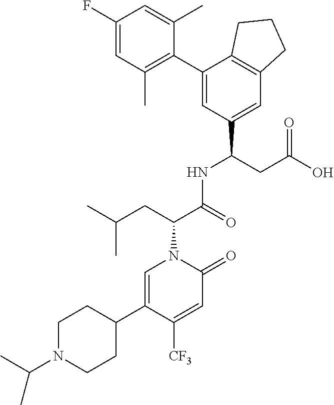 [SMILES: Cc1cc(F)cc(C)c1-c1cc([C@@H](CC(=O)O)NC(=O)[C@@H](CC(C)C)n2cc(C3CCN(C(C)C)CC3)c(C(F)(F)F)cc2=O)cc2c1CCC2] | 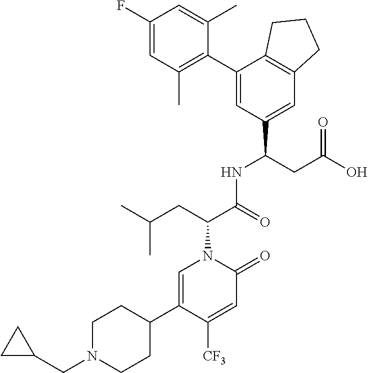 [SMILES: Cc1cc(F)cc(C)c1-c1cc([C@@H](CC(=O)O)NC(=O)[C@@H](CC(C)C)n2cc(C3CCN(CC4CC4)CC3)c(C(F)(F)F)cc2=O)cc2c1CCC2] |
| 250 | 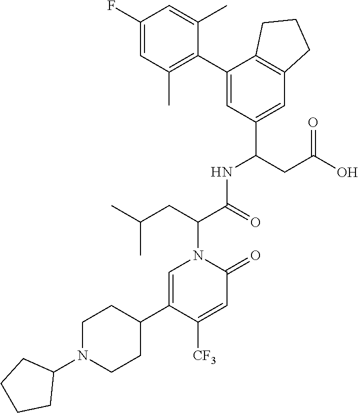 [SMILES: Cc1cc(F)cc(C)c1-c1cc(C(CC(=O)O)NC(=O)C(CC(C)C)n2cc(C3CCN(C4CCCC4)CC3)c(C(F)(F)F)cc2=O)cc2c1CCC2] | 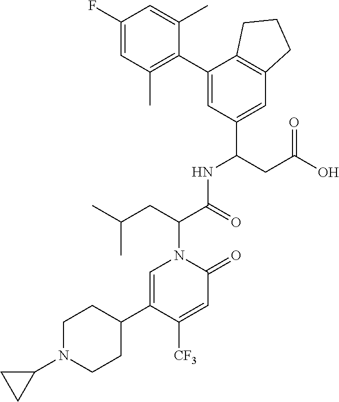 [SMILES: Cc1cc(F)cc(C)c1-c1cc(C(CC(=O)O)NC(=O)C(CC(C)C)n2cc(C3CCN(C4CC4)CC3)c(C(F)(F)F)cc2=O)cc2c1CCC2] |
| 251 | 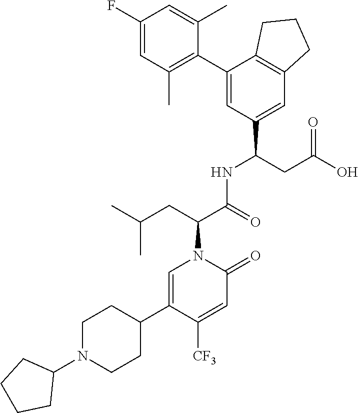 [SMILES: Cc1cc(F)cc(C)c1-c1cc([C@@H](CC(=O)O)NC(=O)[C@H](CC(C)C)n2cc(C3CCN(C4CCCC4)CC3)c(C(F)(F)F)cc2=O)cc2c1CCC2] | 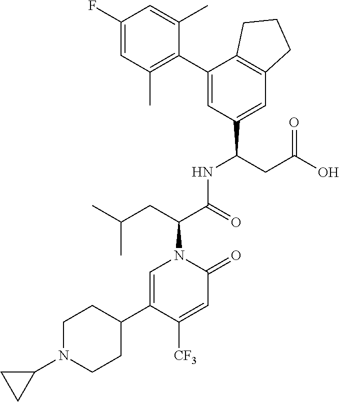 [SMILES: Cc1cc(F)cc(C)c1-c1cc([C@@H](CC(=O)O)NC(=O)[C@H](CC(C)C)n2cc(C3CCN(C4CC4)CC3)c(C(F)(F)F)cc2=O)cc2c1CCC2] |
| 252 | 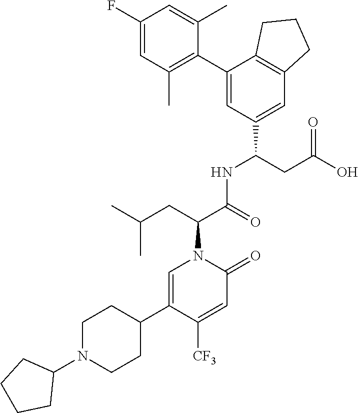 [SMILES: Cc1cc(F)cc(C)c1-c1cc([C@H](CC(=O)O)NC(=O)[C@H](CC(C)C)n2cc(C3CCN(C4CCCC4)CC3)c(C(F)(F)F)cc2=O)cc2c1CCC2] | 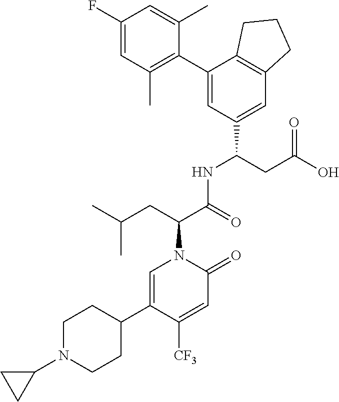 [SMILES: Cc1cc(F)cc(C)c1-c1cc([C@H](CC(=O)O)NC(=O)[C@H](CC(C)C)n2cc(C3CCN(C4CC4)CC3)c(C(F)(F)F)cc2=O)cc2c1CCC2] |
| 253 | 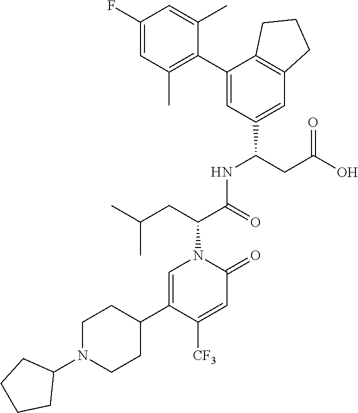 [SMILES: Cc1cc(F)cc(C)c1-c1cc([C@H](CC(=O)O)NC(=O)[C@@H](CC(C)C)n2cc(C3CCN(C4CCCC4)CC3)c(C(F)(F)F)cc2=O)cc2c1CCC2] | 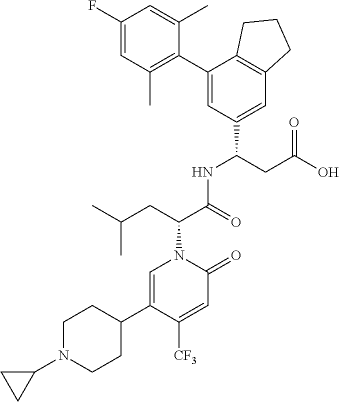 [SMILES: Cc1cc(F)cc(C)c1-c1cc([C@H](CC(=O)O)NC(=O)[C@@H](CC(C)C)n2cc(C3CCN(C4CC4)CC3)c(C(F)(F)F)cc2=O)cc2c1CCC2] |
| 254 | 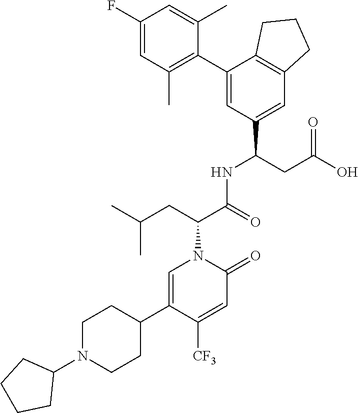 [SMILES: Cc1cc(F)cc(C)c1-c1cc([C@@H](CC(=O)O)NC(=O)[C@@H](CC(C)C)n2cc(C3CCN(C4CCCC4)CC3)c(C(F)(F)F)cc2=O)cc2c1CCC2] | 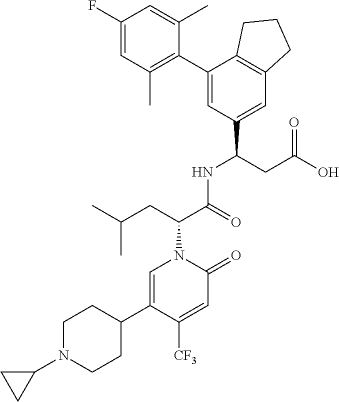 [SMILES: Cc1cc(F)cc(C)c1-c1cc([C@@H](CC(=O)O)NC(=O)[C@@H](CC(C)C)n2cc(C3CCN(C4CC4)CC3)c(C(F)(F)F)cc2=O)cc2c1CCC2] |
| 255 | 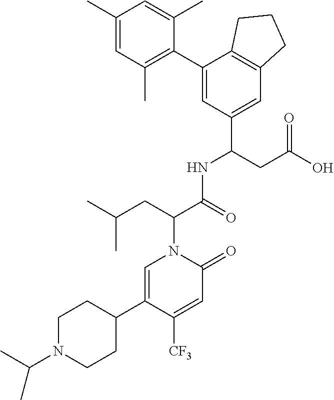 [SMILES: Cc1cc(C)c(-c2cc(C(CC(=O)O)NC(=O)C(CC(C)C)n3cc(C4CCN(C(C)C)CC4)c(C(F)(F)F)cc3=O)cc3c2CCC3)c(C)c1] | 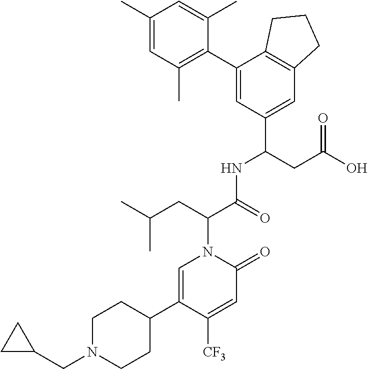 [SMILES: Cc1cc(C)c(-c2cc(C(CC(=O)O)NC(=O)C(CC(C)C)n3cc(C4CCN(CC5CC5)CC4)c(C(F)(F)F)cc3=O)cc3c2CCC3)c(C)c1] |
| 256 | 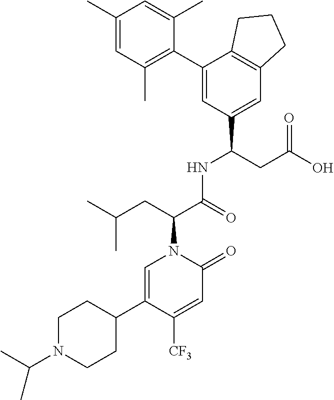 [SMILES: Cc1cc(C)c(-c2cc([C@@H](CC(=O)O)NC(=O)[C@H](CC(C)C)n3cc(C4CCN(C(C)C)CC4)c(C(F)(F)F)cc3=O)cc3c2CCC3)c(C)c1] | 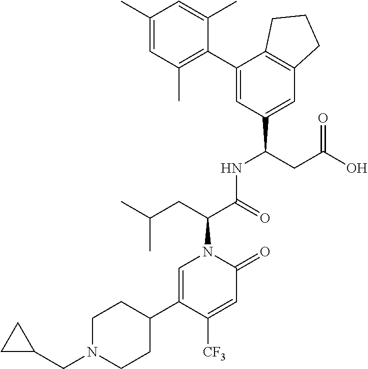 [SMILES: Cc1cc(C)c(-c2cc([C@@H](CC(=O)O)NC(=O)[C@H](CC(C)C)n3cc(C4CCN(CC5CC5)CC4)c(C(F)(F)F)cc3=O)cc3c2CCC3)c(C)c1] |
| 257 | 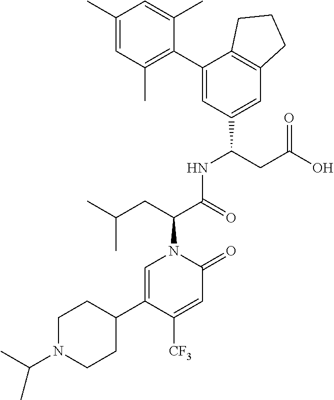 [SMILES: Cc1cc(C)c(-c2cc([C@H](CC(=O)O)NC(=O)[C@H](CC(C)C)n3cc(C4CCN(C(C)C)CC4)c(C(F)(F)F)cc3=O)cc3c2CCC3)c(C)c1] | 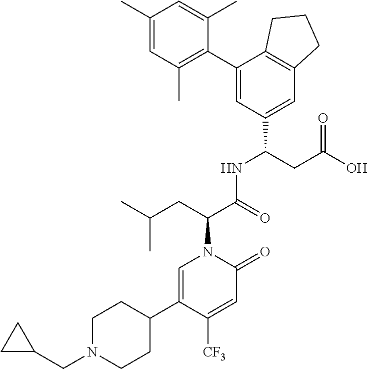 [SMILES: Cc1cc(C)c(-c2cc([C@H](CC(=O)O)NC(=O)[C@H](CC(C)C)n3cc(C4CCN(CC5CC5)CC4)c(C(F)(F)F)cc3=O)cc3c2CCC3)c(C)c1] |
| 258 | 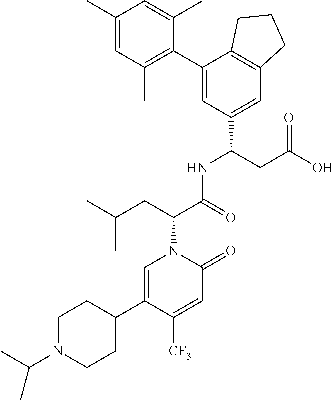 [SMILES: Cc1cc(C)c(-c2cc([C@H](CC(=O)O)NC(=O)[C@@H](CC(C)C)n3cc(C4CCN(C(C)C)CC4)c(C(F)(F)F)cc3=O)cc3c2CCC3)c(C)c1] | 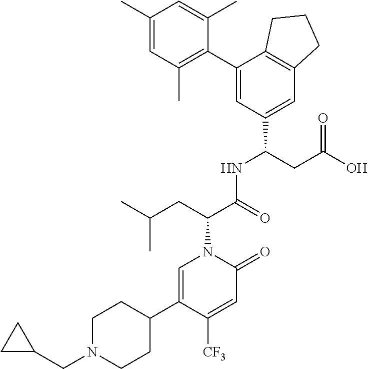 [SMILES: Cc1cc(C)c(-c2cc([C@H](CC(=O)O)NC(=O)[C@@H](CC(C)C)n3cc(C4CCN(CC5CC5)CC4)c(C(F)(F)F)cc3=O)cc3c2CCC3)c(C)c1] |
| 259 | 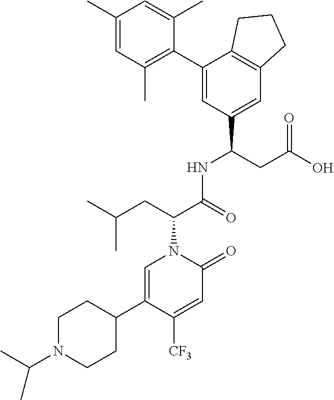 [SMILES: Cc1cc(C)c(-c2cc([C@@H](CC(=O)O)NC(=O)[C@@H](CC(C)C)n3cc(C4CCN(C(C)C)CC4)c(C(F)(F)F)cc3=O)cc3c2CCC3)c(C)c1] | 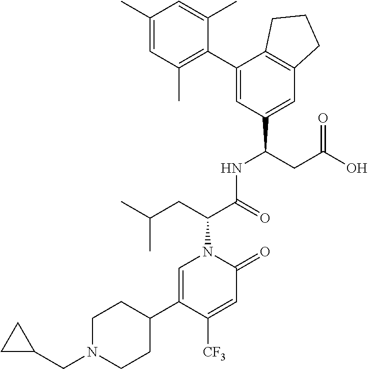 [SMILES: Cc1cc(C)c(-c2cc([C@@H](CC(=O)O)NC(=O)[C@@H](CC(C)C)n3cc(C4CCN(CC5CC5)CC4)c(C(F)(F)F)cc3=O)cc3c2CCC3)c(C)c1] |
| 260 | 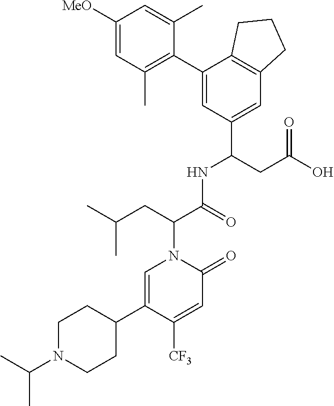 [SMILES: COc1cc(C)c(-c2cc(C(CC(=O)O)NC(=O)C(CC(C)C)n3cc(C4CCN(C(C)C)CC4)c(C(F)(F)F)cc3=O)cc3c2CCC3)c(C)c1] | 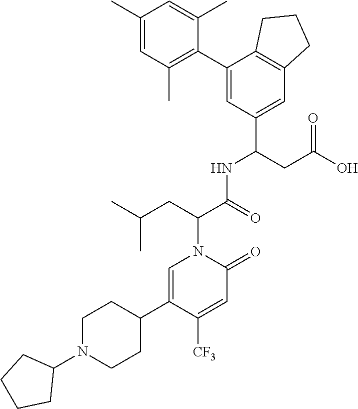 [SMILES: Cc1cc(C)c(-c2cc(C(CC(=O)O)NC(=O)C(CC(C)C)n3cc(C4CCN(C5CCCC5)CC4)c(C(F)(F)F)cc3=O)cc3c2CCC3)c(C)c1] |
| 261 | 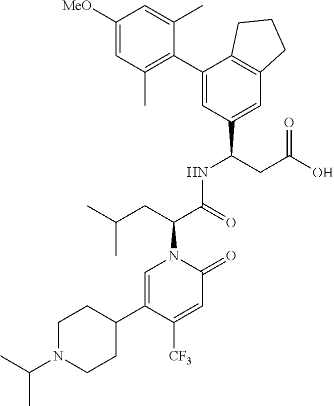 [SMILES: COc1cc(C)c(-c2cc([C@@H](CC(=O)O)NC(=O)[C@H](CC(C)C)n3cc(C4CCN(C(C)C)CC4)c(C(F)(F)F)cc3=O)cc3c2CCC3)c(C)c1] | 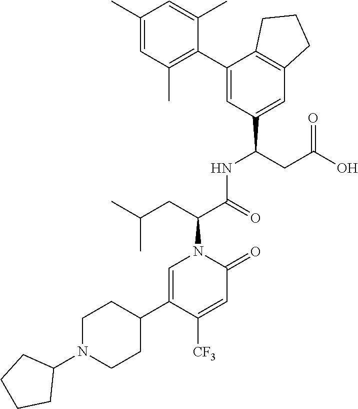 [SMILES: Cc1cc(C)c(-c2cc([C@@H](CC(=O)O)NC(=O)[C@H](CC(C)C)n3cc(C4CCN(C5CCCC5)CC4)c(C(F)(F)F)cc3=O)cc3c2CCC3)c(C)c1] |
| 262 | 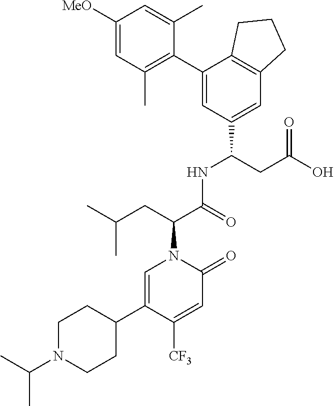 [SMILES: COc1cc(C)c(-c2cc([C@H](CC(=O)O)NC(=O)[C@H](CC(C)C)n3cc(C4CCN(C(C)C)CC4)c(C(F)(F)F)cc3=O)cc3c2CCC3)c(C)c1] | 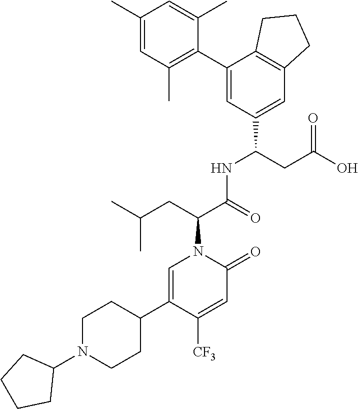 [SMILES: Cc1cc(C)c(-c2cc([C@H](CC(=O)O)NC(=O)[C@H](CC(C)C)n3cc(C4CCN(C5CCCC5)CC4)c(C(F)(F)F)cc3=O)cc3c2CCC3)c(C)c1] |
| 263 | 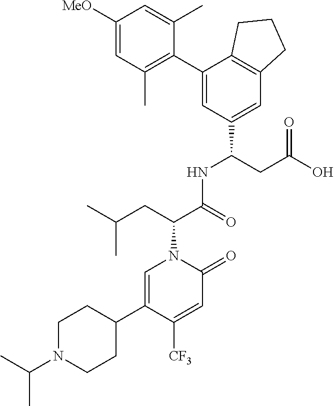 [SMILES: COc1cc(C)c(-c2cc([C@H](CC(=O)O)NC(=O)[C@@H](CC(C)C)n3cc(C4CCN(C(C)C)CC4)c(C(F)(F)F)cc3=O)cc3c2CCC3)c(C)c1] | 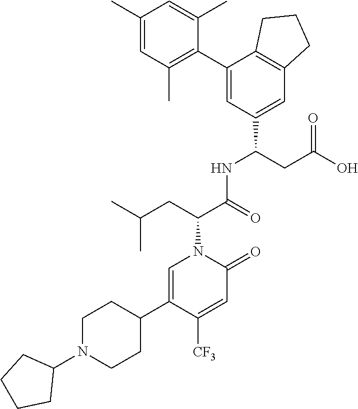 [SMILES: Cc1cc(C)c(-c2cc([C@H](CC(=O)O)NC(=O)[C@@H](CC(C)C)n3cc(C4CCN(C5CCCC5)CC4)c(C(F)(F)F)cc3=O)cc3c2CCC3)c(C)c1] |
| 264 | 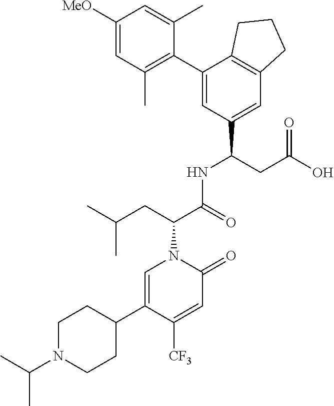 [SMILES: COc1cc(C)c(-c2cc([C@@H](CC(=O)O)NC(=O)[C@@H](CC(C)C)n3cc(C4CCN(C(C)C)CC4)c(C(F)(F)F)cc3=O)cc3c2CCC3)c(C)c1] | 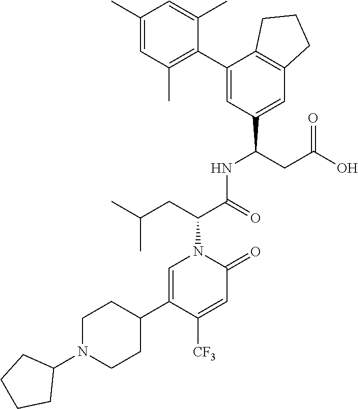 [SMILES: Cc1cc(C)c(-c2cc([C@@H](CC(=O)O)NC(=O)[C@@H](CC(C)C)n3cc(C4CCN(C5CCCC5)CC4)c(C(F)(F)F)cc3=O)cc3c2CCC3)c(C)c1] |
| 265 | 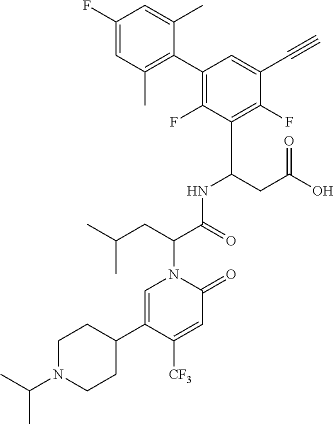 [SMILES: C#Cc1cc(-c2c(C)cc(F)cc2C)c(F)c(C(CC(=O)O)NC(=O)C(CC(C)C)n2cc(C3CCN(C(C)C)CC3)c(C(F)(F)F)cc2=O)c1F] | 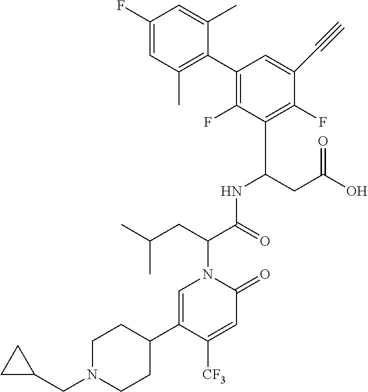 [SMILES: C#Cc1cc(-c2c(C)cc(F)cc2C)c(F)c(C(CC(=O)O)NC(=O)C(CC(C)C)n2cc(C3CCN(CC4CC4)CC3)c(C(F)(F)F)cc2=O)c1F] |
| 266 | 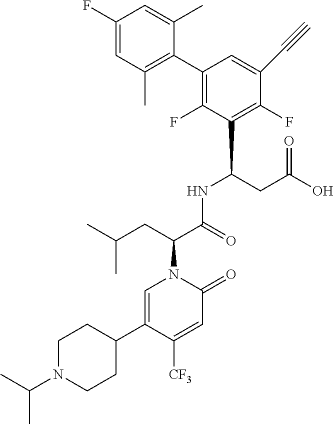 [SMILES: C#Cc1cc(-c2c(C)cc(F)cc2C)c(F)c([C@@H](CC(=O)O)NC(=O)[C@H](CC(C)C)n2cc(C3CCN(C(C)C)CC3)c(C(F)(F)F)cc2=O)c1F] | 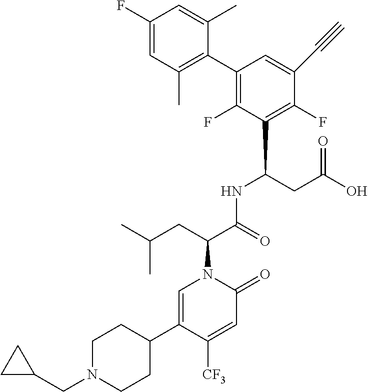 [SMILES: C#Cc1cc(-c2c(C)cc(F)cc2C)c(F)c([C@@H](CC(=O)O)NC(=O)[C@H](CC(C)C)n2cc(C3CCN(CC4CC4)CC3)c(C(F)(F)F)cc2=O)c1F] |
| 267 | 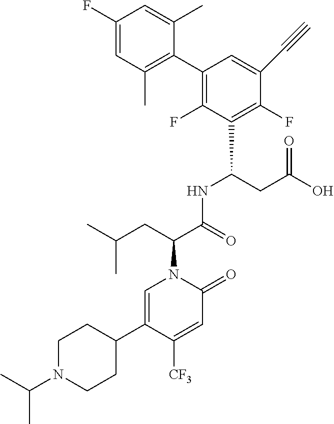 [SMILES: C#Cc1cc(-c2c(C)cc(F)cc2C)c(F)c([C@H](CC(=O)O)NC(=O)[C@H](CC(C)C)n2cc(C3CCN(C(C)C)CC3)c(C(F)(F)F)cc2=O)c1F] | 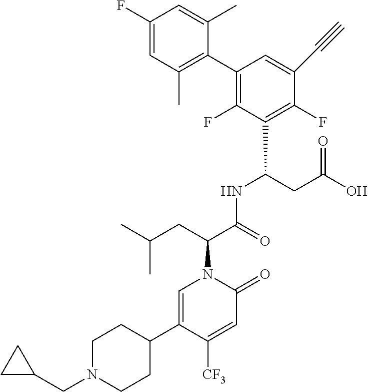 [SMILES: C#Cc1cc(-c2c(C)cc(F)cc2C)c(F)c([C@H](CC(=O)O)NC(=O)[C@H](CC(C)C)n2cc(C3CCN(CC4CC4)CC3)c(C(F)(F)F)cc2=O)c1F] |
| 268 | 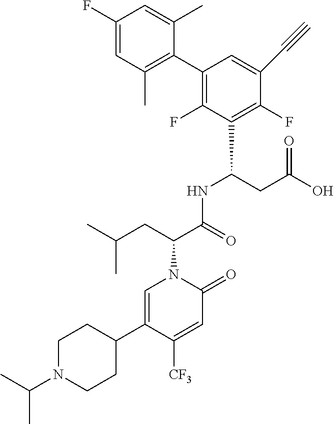 [SMILES: C#Cc1cc(-c2c(C)cc(F)cc2C)c(F)c([C@H](CC(=O)O)NC(=O)[C@@H](CC(C)C)n2cc(C3CCN(C(C)C)CC3)c(C(F)(F)F)cc2=O)c1F] | 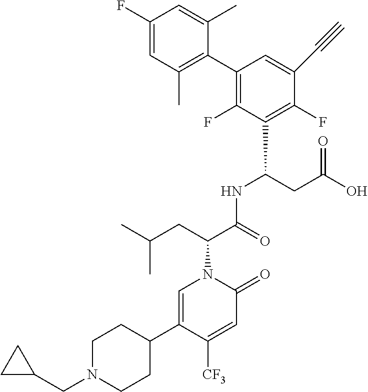 [SMILES: C#Cc1cc(-c2c(C)cc(F)cc2C)c(F)c([C@H](CC(=O)O)NC(=O)[C@@H](CC(C)C)n2cc(C3CCN(CC4CC4)CC3)c(C(F)(F)F)cc2=O)c1F] |
| 269 | 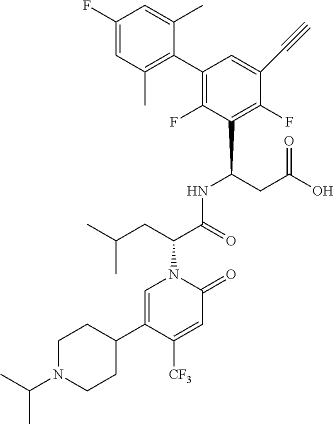 [SMILES: C#Cc1cc(-c2c(C)cc(F)cc2C)c(F)c([C@@H](CC(=O)O)NC(=O)[C@@H](CC(C)C)n2cc(C3CCN(C(C)C)CC3)c(C(F)(F)F)cc2=O)c1F] | 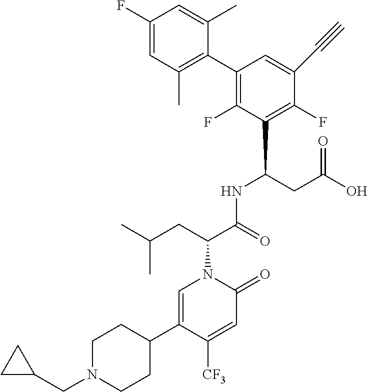 [SMILES: C#Cc1cc(-c2c(C)cc(F)cc2C)c(F)c([C@@H](CC(=O)O)NC(=O)[C@@H](CC(C)C)n2cc(C3CCN(CC4CC4)CC3)c(C(F)(F)F)cc2=O)c1F] |
| 270 | 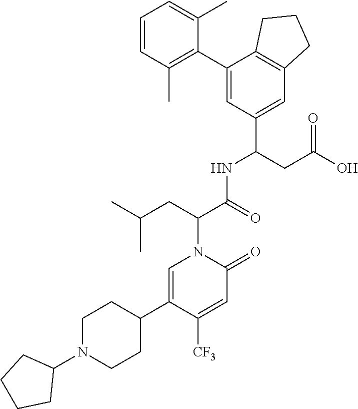 [SMILES: Cc1cccc(C)c1-c1cc(C(CC(=O)O)NC(=O)C(CC(C)C)n2cc(C3CCN(C4CCCC4)CC3)c(C(F)(F)F)cc2=O)cc2c1CCC2] | 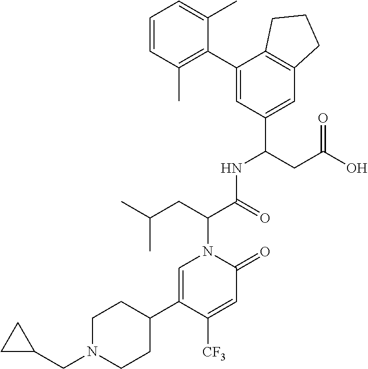 [SMILES: Cc1cccc(C)c1-c1cc(C(CC(=O)O)NC(=O)C(CC(C)C)n2cc(C3CCN(CC4CC4)CC3)c(C(F)(F)F)cc2=O)cc2c1CCC2] |
| 271 | 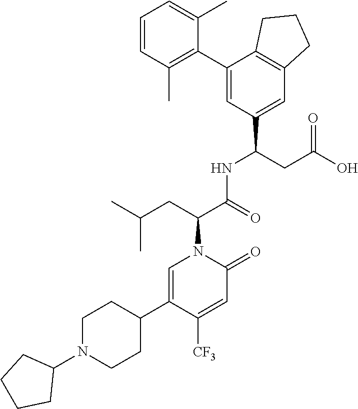 [SMILES: Cc1cccc(C)c1-c1cc([C@@H](CC(=O)O)NC(=O)[C@H](CC(C)C)n2cc(C3CCN(C4CCCC4)CC3)c(C(F)(F)F)cc2=O)cc2c1CCC2] | 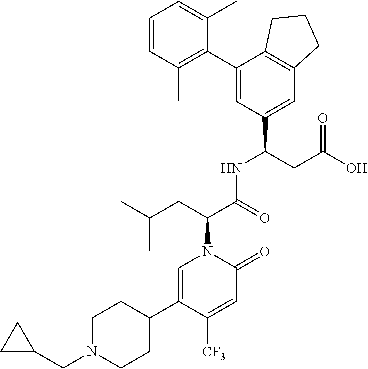 [SMILES: Cc1cccc(C)c1-c1cc([C@@H](CC(=O)O)NC(=O)[C@H](CC(C)C)n2cc(C3CCN(CC4CC4)CC3)c(C(F)(F)F)cc2=O)cc2c1CCC2] |
| 272 | 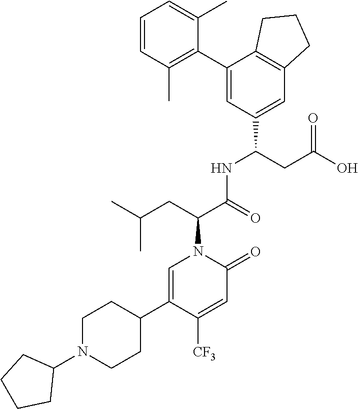 [SMILES: Cc1cccc(C)c1-c1cc([C@H](CC(=O)O)NC(=O)[C@H](CC(C)C)n2cc(C3CCN(C4CCCC4)CC3)c(C(F)(F)F)cc2=O)cc2c1CCC2] | 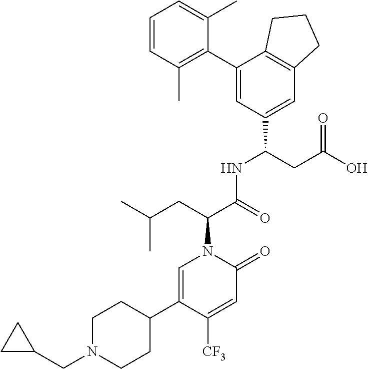 [SMILES: Cc1cccc(C)c1-c1cc([C@H](CC(=O)O)NC(=O)[C@H](CC(C)C)n2cc(C3CCN(CC4CC4)CC3)c(C(F)(F)F)cc2=O)cc2c1CCC2] |
| 273 | 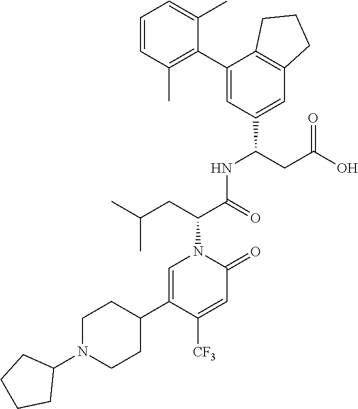 [SMILES: Cc1cccc(C)c1-c1cc([C@H](CC(=O)O)NC(=O)[C@@H](CC(C)C)n2cc(C3CCN(C4CCCC4)CC3)c(C(F)(F)F)cc2=O)cc2c1CCC2] | 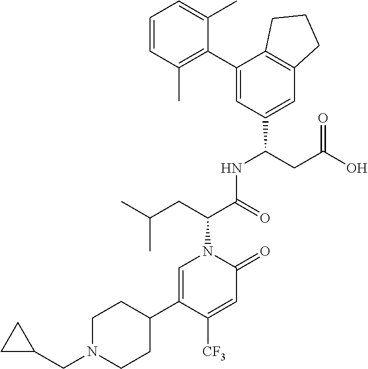 [SMILES: Cc1cccc(C)c1-c1cc([C@H](CC(=O)O)NC(=O)[C@@H](CC(C)C)n2cc(C3CCN(CC4CC4)CC3)c(C(F)(F)F)cc2=O)cc2c1CCC2] |
| 274 | 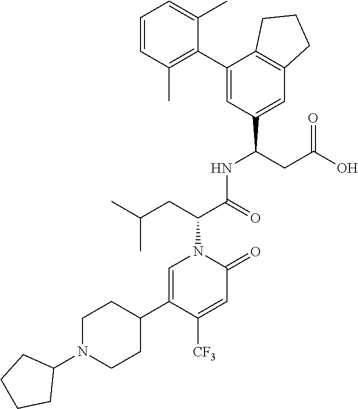 [SMILES: Cc1cccc(C)c1-c1cc([C@@H](CC(=O)O)NC(=O)[C@@H](CC(C)C)n2cc(C3CCN(C4CCCC4)CC3)c(C(F)(F)F)cc2=O)cc2c1CCC2] | 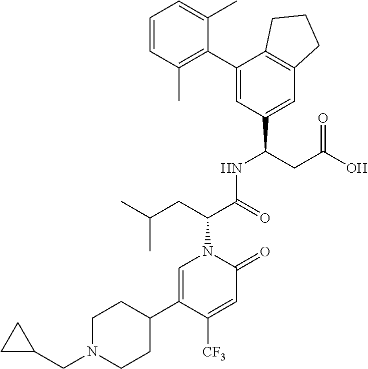 [SMILES: Cc1cccc(C)c1-c1cc([C@@H](CC(=O)O)NC(=O)[C@@H](CC(C)C)n2cc(C3CCN(CC4CC4)CC3)c(C(F)(F)F)cc2=O)cc2c1CCC2] |
| 275 | 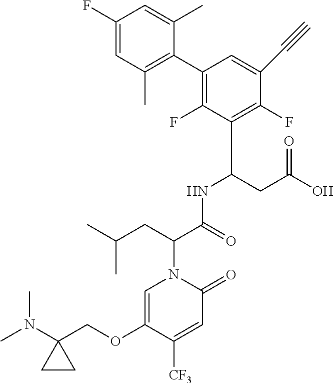 [SMILES: C#Cc1cc(-c2c(C)cc(F)cc2C)c(F)c(C(CC(=O)O)NC(=O)C(CC(C)C)n2cc(OCC3(N(C)C)CC3)c(C(F)(F)F)cc2=O)c1F] | 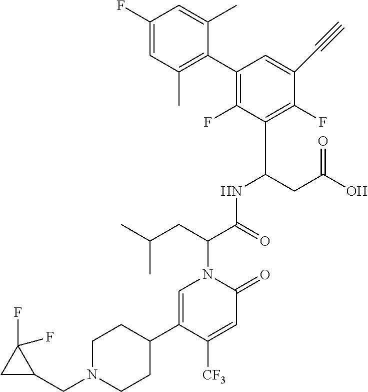 [SMILES: C#Cc1cc(-c2c(C)cc(F)cc2C)c(F)c(C(CC(=O)O)NC(=O)C(CC(C)C)n2cc(C3CCN(CC4CC4(F)F)CC3)c(C(F)(F)F)cc2=O)c1F] |
| 276 | 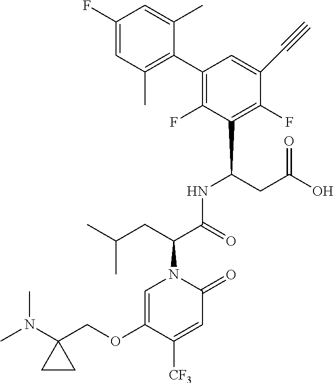 [SMILES: C#Cc1cc(-c2c(C)cc(F)cc2C)c(F)c([C@@H](CC(=O)O)NC(=O)[C@H](CC(C)C)n2cc(OCC3(N(C)C)CC3)c(C(F)(F)F)cc2=O)c1F] | 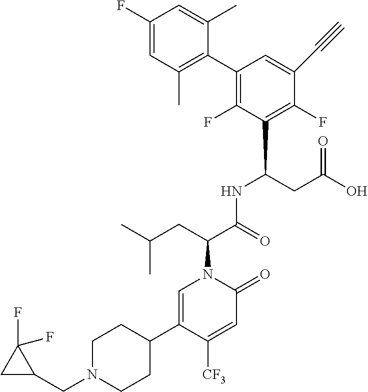 [SMILES: C#Cc1cc(-c2c(C)cc(F)cc2C)c(F)c([C@@H](CC(=O)O)NC(=O)[C@H](CC(C)C)n2cc(C3CCN(CC4CC4(F)F)CC3)c(C(F)(F)F)cc2=O)c1F] |
| 277 | 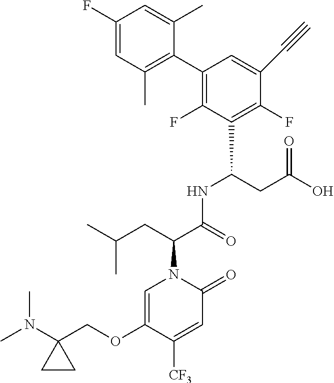 [SMILES: C#Cc1cc(-c2c(C)cc(F)cc2C)c(F)c([C@H](CC(=O)O)NC(=O)[C@H](CC(C)C)n2cc(OCC3(N(C)C)CC3)c(C(F)(F)F)cc2=O)c1F] | 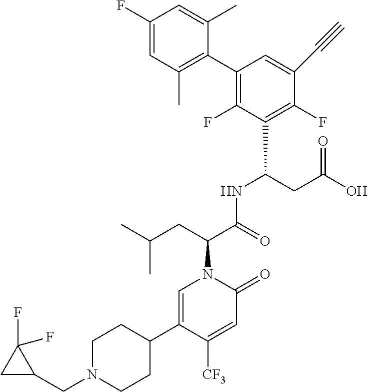 [SMILES: C#Cc1cc(-c2c(C)cc(F)cc2C)c(F)c([C@H](CC(=O)O)NC(=O)[C@H](CC(C)C)n2cc(C3CCN(CC4CC4(F)F)CC3)c(C(F)(F)F)cc2=O)c1F] |
| 278 | 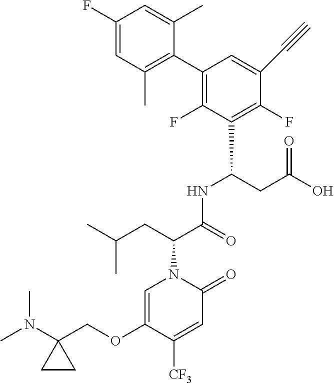 [SMILES: C#Cc1cc(-c2c(C)cc(F)cc2C)c(F)c([C@H](CC(=O)O)NC(=O)[C@@H](CC(C)C)n2cc(OCC3(N(C)C)CC3)c(C(F)(F)F)cc2=O)c1F] | 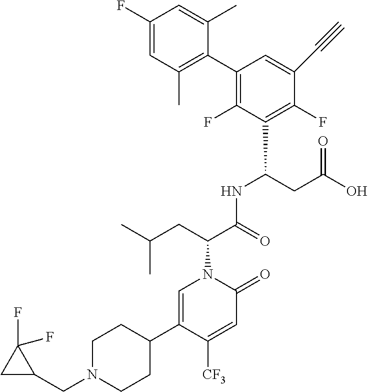 [SMILES: C#Cc1cc(-c2c(C)cc(F)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] |
| 279 | 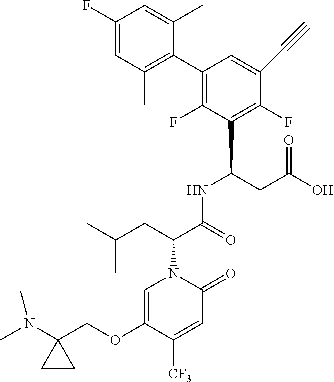 [SMILES: C#Cc1cc(-c2c(C)cc(F)cc2C)c(F)c([C@@H](CC(=O)O)NC(=O)[C@@H](CC(C)C)n2cc(OCC3(N(C)C)CC3)c(C(F)(F)F)cc2=O)c1F] | 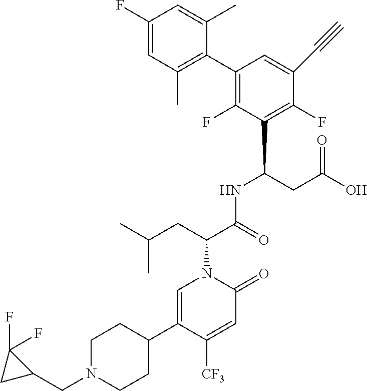 [SMILES: C#Cc1cc(-c2c(C)cc(F)cc2C)c(F)c([C@@H](CC(=O)O)NC(=O)[C@@H](CC(C)C)n2cc(C3CCN(CC4CC4(F)F)CC3)c(C(F)(F)F)cc2=O)c1F] |
| 280 | 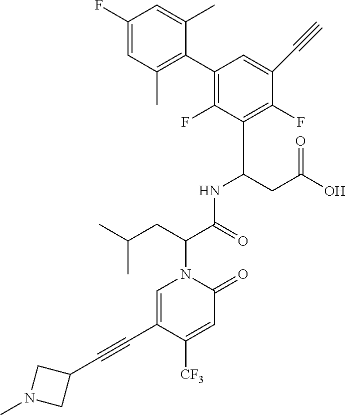 [SMILES: C#Cc1cc(-c2c(C)cc(F)cc2C)c(F)c(C(CC(=O)O)NC(=O)C(CC(C)C)n2cc(C#CC3CN(C)C3)c(C(F)(F)F)cc2=O)c1F] | 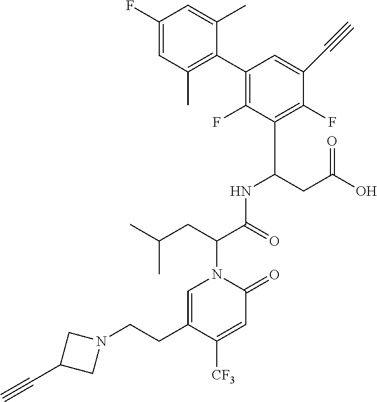 [SMILES: C#Cc1cc(-c2c(C)cc(F)cc2C)c(F)c(C(CC(=O)O)NC(=O)C(CC(C)C)n2cc(CCN3CC(C#C)C3)c(C(F)(F)F)cc2=O)c1F] |
| 281 | 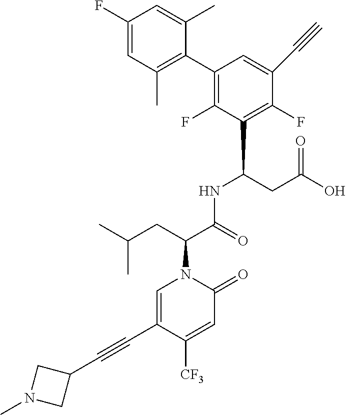 [SMILES: C#Cc1cc(-c2c(C)cc(F)cc2C)c(F)c([C@@H](CC(=O)O)NC(=O)[C@H](CC(C)C)n2cc(C#CC3CN(C)C3)c(C(F)(F)F)cc2=O)c1F] | 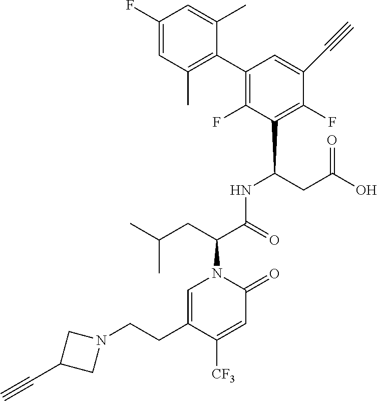 [SMILES: C#Cc1cc(-c2c(C)cc(F)cc2C)c(F)c([C@@H](CC(=O)O)NC(=O)[C@H](CC(C)C)n2cc(CCN3CC(C#C)C3)c(C(F)(F)F)cc2=O)c1F] |
| 282 | 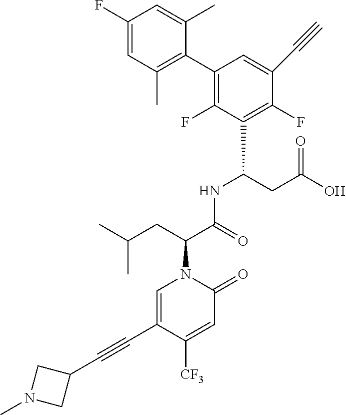 [SMILES: C#Cc1cc(-c2c(C)cc(F)cc2C)c(F)c([C@H](CC(=O)O)NC(=O)[C@H](CC(C)C)n2cc(C#CC3CN(C)C3)c(C(F)(F)F)cc2=O)c1F] | 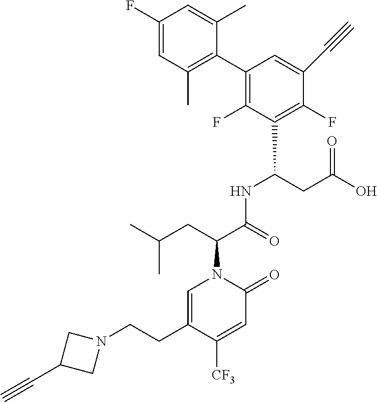 [SMILES: C#Cc1cc(-c2c(C)cc(F)cc2C)c(F)c([C@H](CC(=O)O)NC(=O)[C@H](CC(C)C)n2cc(CCN3CC(C#C)C3)c(C(F)(F)F)cc2=O)c1F] |
| 283 | 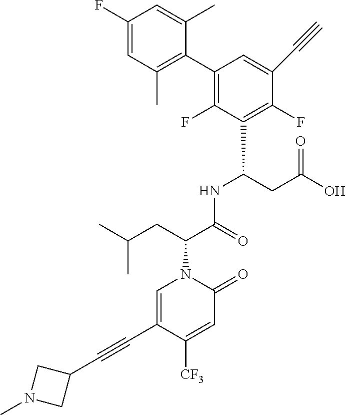 [SMILES: C#Cc1cc(-c2c(C)cc(F)cc2C)c(F)c([C@H](CC(=O)O)NC(=O)[C@@H](CC(C)C)n2cc(C#CC3CN(C)C3)c(C(F)(F)F)cc2=O)c1F] | 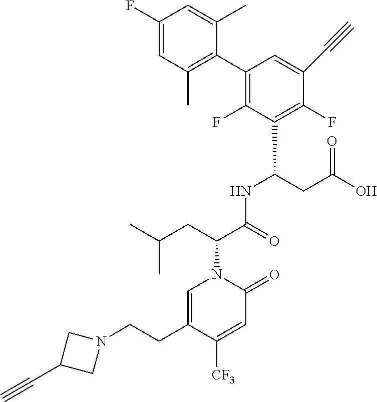 [SMILES: C#Cc1cc(-c2c(C)cc(F)cc2C)c(F)c([C@H](CC(=O)O)NC(=O)[C@@H](CC(C)C)n2cc(CCN3CC(C#C)C3)c(C(F)(F)F)cc2=O)c1F] |
| 284 | 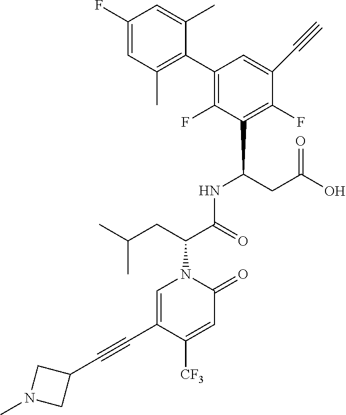 [SMILES: C#Cc1cc(-c2c(C)cc(F)cc2C)c(F)c([C@@H](CC(=O)O)NC(=O)[C@@H](CC(C)C)n2cc(C#CC3CN(C)C3)c(C(F)(F)F)cc2=O)c1F] | 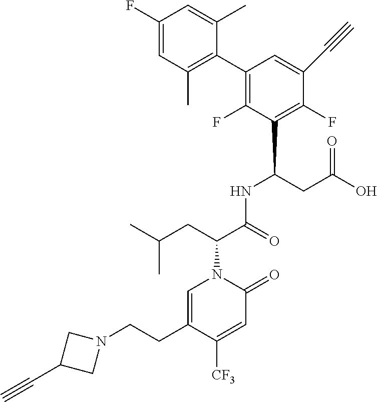 [SMILES: C#Cc1cc(-c2c(C)cc(F)cc2C)c(F)c([C@@H](CC(=O)O)NC(=O)[C@@H](CC(C)C)n2cc(CCN3CC(C#C)C3)c(C(F)(F)F)cc2=O)c1F] |
| 285 | 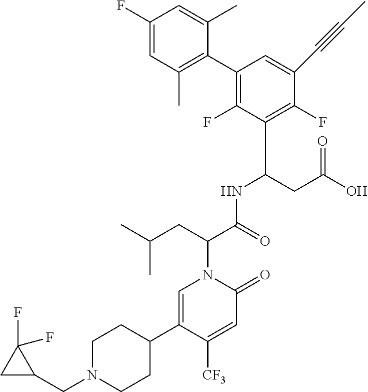 [SMILES: CC#Cc1cc(-c2c(C)cc(F)cc2C)c(F)c(C(CC(=O)O)NC(=O)C(CC(C)C)n2cc(C3CCN(CC4CC4(F)F)CC3)c(C(F)(F)F)cc2=O)c1F] | 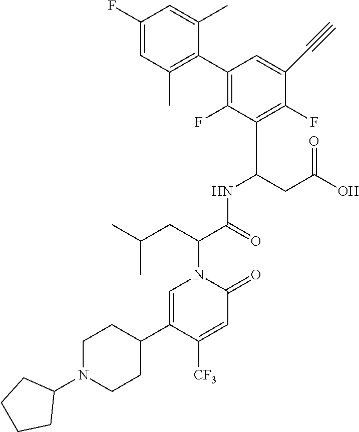 [SMILES: C#Cc1cc(-c2c(C)cc(F)cc2C)c(F)c(C(CC(=O)O)NC(=O)C(CC(C)C)n2cc(C3CCN(C4CCCC4)CC3)c(C(F)(F)F)cc2=O)c1F] |
| 286 | 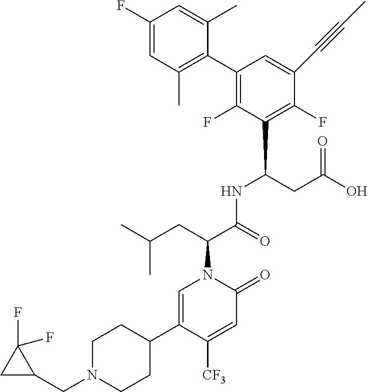 [SMILES: CC#Cc1cc(-c2c(C)cc(F)cc2C)c(F)c([C@@H](CC(=O)O)NC(=O)[C@H](CC(C)C)n2cc(C3CCN(CC4CC4(F)F)CC3)c(C(F)(F)F)cc2=O)c1F] | 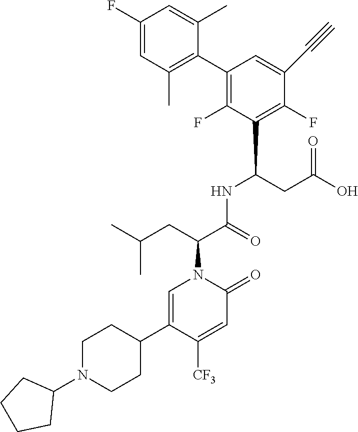 [SMILES: C#Cc1cc(-c2c(C)cc(F)cc2C)c(F)c([C@@H](CC(=O)O)NC(=O)[C@H](CC(C)C)n2cc(C3CCN(C4CCCC4)CC3)c(C(F)(F)F)cc2=O)c1F] |
| 287 | 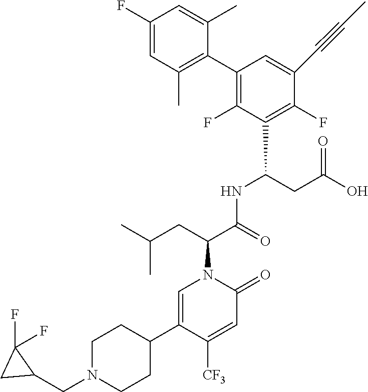 [SMILES: CC#Cc1cc(-c2c(C)cc(F)cc2C)c(F)c([C@H](CC(=O)O)NC(=O)[C@H](CC(C)C)n2cc(C3CCN(CC4CC4(F)F)CC3)c(C(F)(F)F)cc2=O)c1F] | 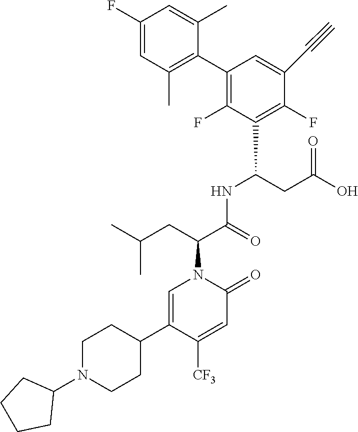 [SMILES: C#Cc1cc(-c2c(C)cc(F)cc2C)c(F)c([C@H](CC(=O)O)NC(=O)[C@H](CC(C)C)n2cc(C3CCN(C4CCCC4)CC3)c(C(F)(F)F)cc2=O)c1F] |
| 288 | 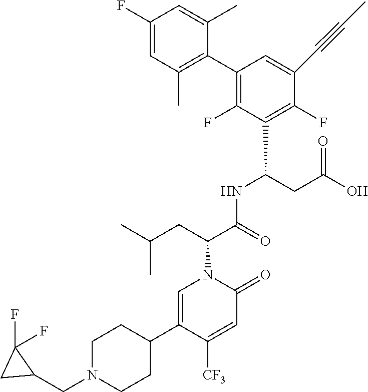 [SMILES: CC#Cc1cc(-c2c(C)cc(F)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] | 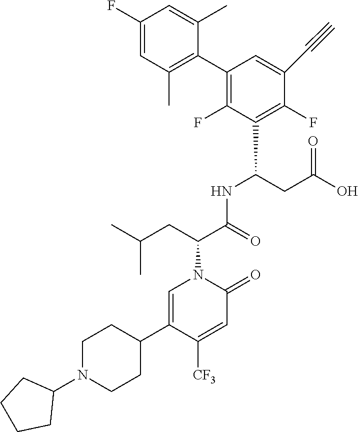 [SMILES: C#Cc1cc(-c2c(C)cc(F)cc2C)c(F)c([C@H](CC(=O)O)NC(=O)[C@@H](CC(C)C)n2cc(C3CCN(C4CCCC4)CC3)c(C(F)(F)F)cc2=O)c1F] |
| 289 | 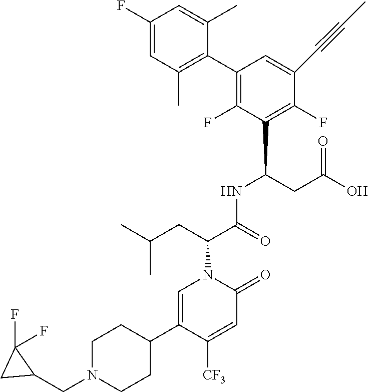 [SMILES: CC#Cc1cc(-c2c(C)cc(F)cc2C)c(F)c([C@@H](CC(=O)O)NC(=O)[C@@H](CC(C)C)n2cc(C3CCN(CC4CC4(F)F)CC3)c(C(F)(F)F)cc2=O)c1F] | 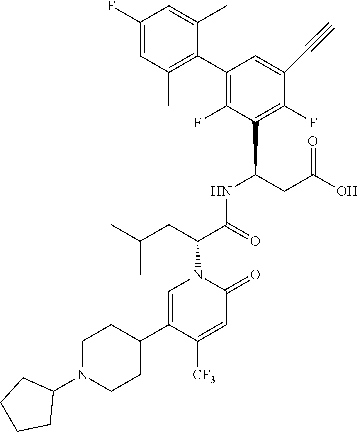 [SMILES: C#Cc1cc(-c2c(C)cc(F)cc2C)c(F)c([C@@H](CC(=O)O)NC(=O)[C@@H](CC(C)C)n2cc(C3CCN(C4CCCC4)CC3)c(C(F)(F)F)cc2=O)c1F] |
| 290 | 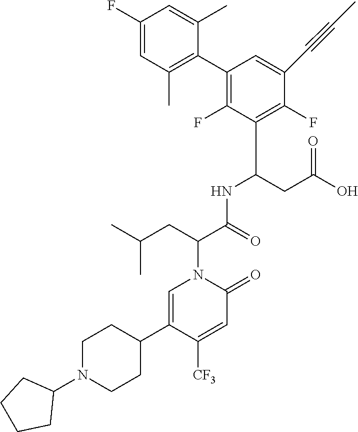 [SMILES: CC#Cc1cc(-c2c(C)cc(F)cc2C)c(F)c(C(CC(=O)O)NC(=O)C(CC(C)C)n2cc(C3CCN(C4CCCC4)CC3)c(C(F)(F)F)cc2=O)c1F] | 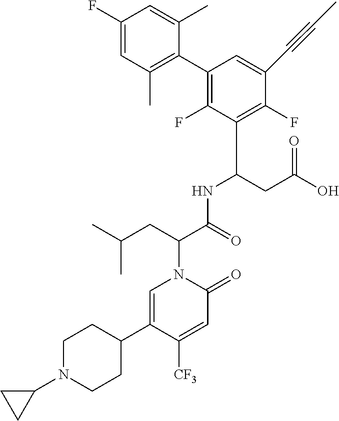 [SMILES: CC#Cc1cc(-c2c(C)cc(F)cc2C)c(F)c(C(CC(=O)O)NC(=O)C(CC(C)C)n2cc(C3CCN(C4CC4)CC3)c(C(F)(F)F)cc2=O)c1F] |
| 291 | 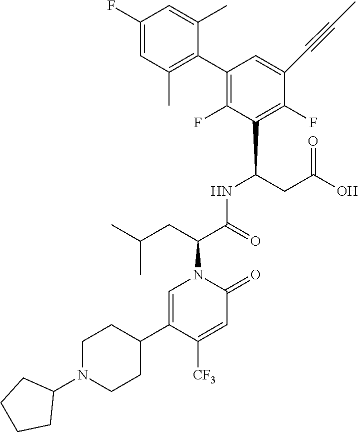 [SMILES: CC#Cc1cc(-c2c(C)cc(F)cc2C)c(F)c([C@@H](CC(=O)O)NC(=O)[C@H](CC(C)C)n2cc(C3CCN(C4CCCC4)CC3)c(C(F)(F)F)cc2=O)c1F] | 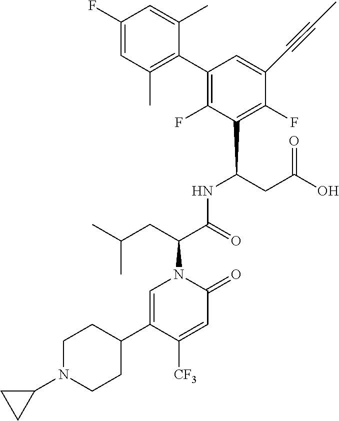 [SMILES: CC#Cc1cc(-c2c(C)cc(F)cc2C)c(F)c([C@@H](CC(=O)O)NC(=O)[C@H](CC(C)C)n2cc(C3CCN(C4CC4)CC3)c(C(F)(F)F)cc2=O)c1F] |
| 292 | 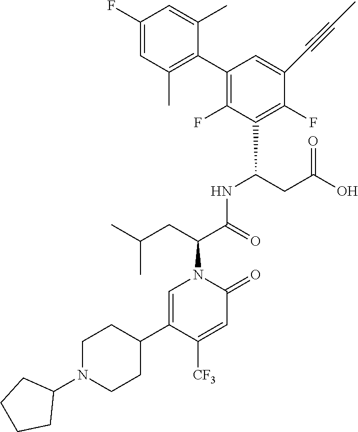 [SMILES: CC#Cc1cc(-c2c(C)cc(F)cc2C)c(F)c([C@H](CC(=O)O)NC(=O)[C@H](CC(C)C)n2cc(C3CCN(C4CCCC4)CC3)c(C(F)(F)F)cc2=O)c1F] | 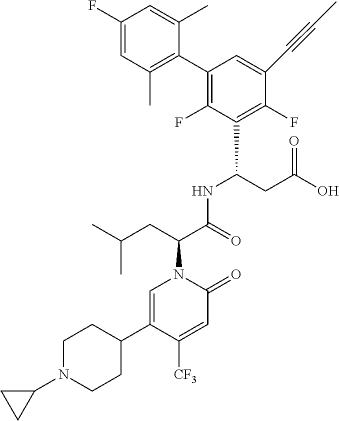 [SMILES: CC#Cc1cc(-c2c(C)cc(F)cc2C)c(F)c([C@H](CC(=O)O)NC(=O)[C@H](CC(C)C)n2cc(C3CCN(C4CC4)CC3)c(C(F)(F)F)cc2=O)c1F] |
| 293 | 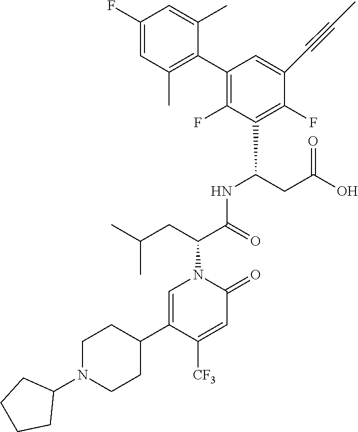 [SMILES: CC#Cc1cc(-c2c(C)cc(F)cc2C)c(F)c([C@H](CC(=O)O)NC(=O)[C@@H](CC(C)C)n2cc(C3CCN(C4CCCC4)CC3)c(C(F)(F)F)cc2=O)c1F] | 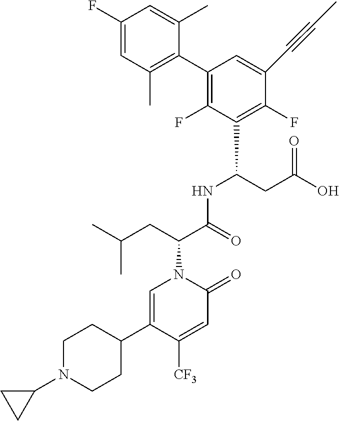 [SMILES: CC#Cc1cc(-c2c(C)cc(F)cc2C)c(F)c([C@H](CC(=O)O)NC(=O)[C@@H](CC(C)C)n2cc(C3CCN(C4CC4)CC3)c(C(F)(F)F)cc2=O)c1F] |
| 294 | 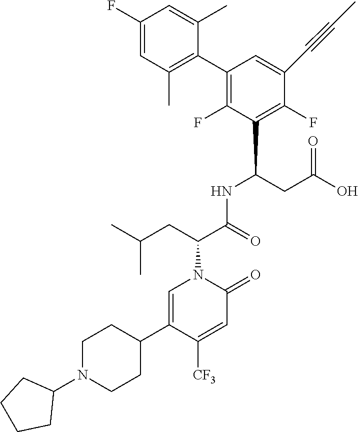 [SMILES: CC#Cc1cc(-c2c(C)cc(F)cc2C)c(F)c([C@@H](CC(=O)O)NC(=O)[C@@H](CC(C)C)n2cc(C3CCN(C4CCCC4)CC3)c(C(F)(F)F)cc2=O)c1F] | 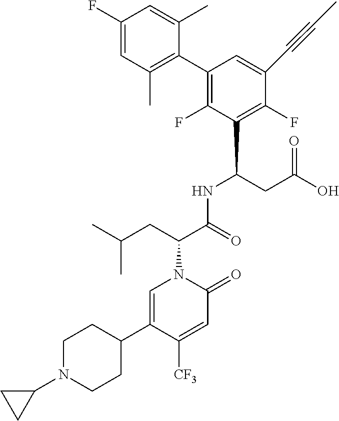 [SMILES: CC#Cc1cc(-c2c(C)cc(F)cc2C)c(F)c([C@@H](CC(=O)O)NC(=O)[C@@H](CC(C)C)n2cc(C3CCN(C4CC4)CC3)c(C(F)(F)F)cc2=O)c1F] |
| 295 | 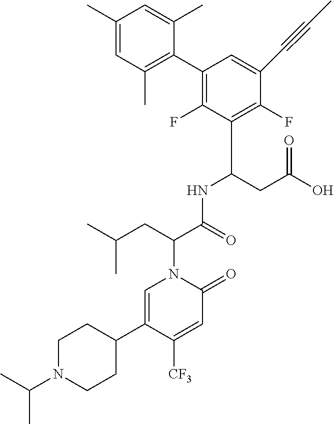 [SMILES: CC#Cc1cc(-c2c(C)cc(C)cc2C)c(F)c(C(CC(=O)O)NC(=O)C(CC(C)C)n2cc(C3CCN(C(C)C)CC3)c(C(F)(F)F)cc2=O)c1F] | 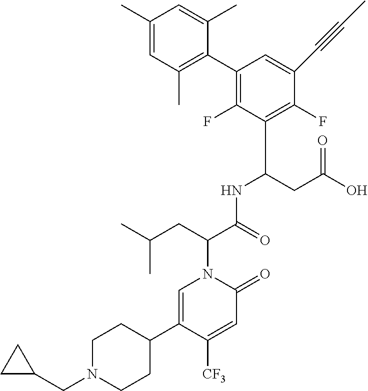 [SMILES: CC#Cc1cc(-c2c(C)cc(C)cc2C)c(F)c(C(CC(=O)O)NC(=O)C(CC(C)C)n2cc(C3CCN(CC4CC4)CC3)c(C(F)(F)F)cc2=O)c1F] |
| 296 | 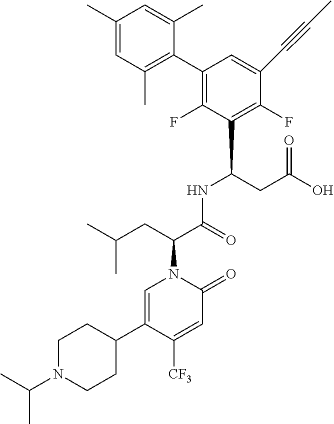 [SMILES: CC#Cc1cc(-c2c(C)cc(C)cc2C)c(F)c([C@@H](CC(=O)O)NC(=O)[C@H](CC(C)C)n2cc(C3CCN(C(C)C)CC3)c(C(F)(F)F)cc2=O)c1F] | 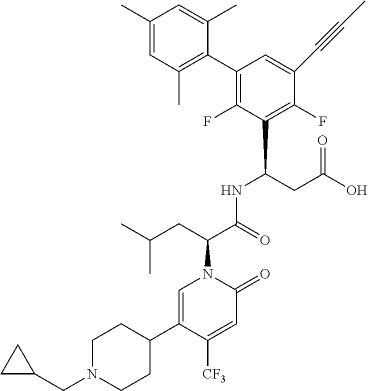 [SMILES: CC#Cc1cc(-c2c(C)cc(C)cc2C)c(F)c([C@@H](CC(=O)O)NC(=O)[C@H](CC(C)C)n2cc(C3CCN(CC4CC4)CC3)c(C(F)(F)F)cc2=O)c1F] |
| 297 | 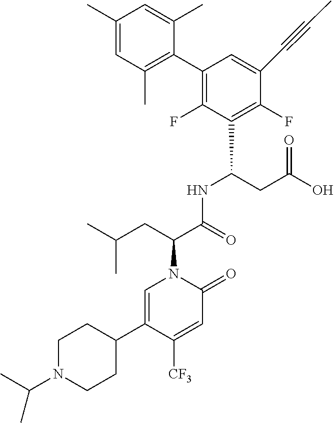 [SMILES: CC#Cc1cc(-c2c(C)cc(C)cc2C)c(F)c([C@H](CC(=O)O)NC(=O)[C@H](CC(C)C)n2cc(C3CCN(C(C)C)CC3)c(C(F)(F)F)cc2=O)c1F] | 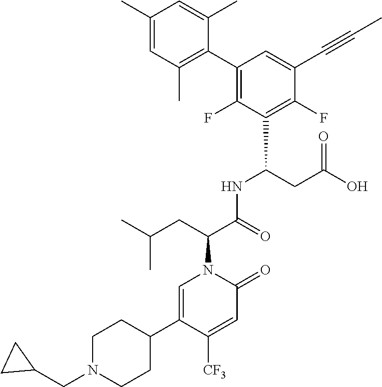 [SMILES: CC#Cc1cc(-c2c(C)cc(C)cc2C)c(F)c([C@H](CC(=O)O)NC(=O)[C@H](CC(C)C)n2cc(C3CCN(CC4CC4)CC3)c(C(F)(F)F)cc2=O)c1F] |
| 298 | 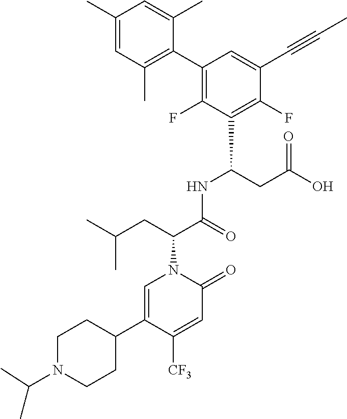 [SMILES: CC#Cc1cc(-c2c(C)cc(C)cc2C)c(F)c([C@H](CC(=O)O)NC(=O)[C@@H](CC(C)C)n2cc(C3CCN(C(C)C)CC3)c(C(F)(F)F)cc2=O)c1F] | 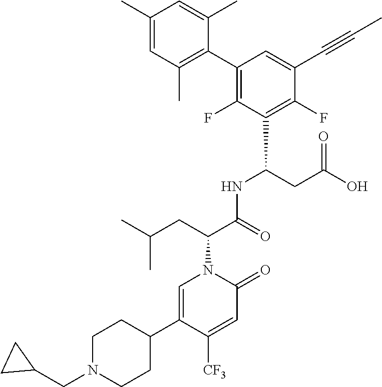 [SMILES: CC#Cc1cc(-c2c(C)cc(C)cc2C)c(F)c([C@H](CC(=O)O)NC(=O)[C@@H](CC(C)C)n2cc(C3CCN(CC4CC4)CC3)c(C(F)(F)F)cc2=O)c1F] |
| 299 | 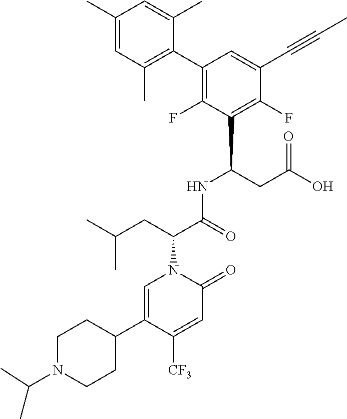 [SMILES: CC#Cc1cc(-c2c(C)cc(C)cc2C)c(F)c([C@@H](CC(=O)O)NC(=O)[C@@H](CC(C)C)n2cc(C3CCN(C(C)C)CC3)c(C(F)(F)F)cc2=O)c1F] | 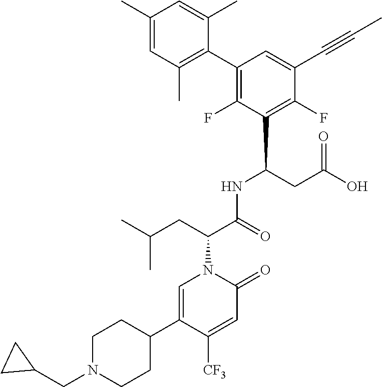 [SMILES: CC#Cc1cc(-c2c(C)cc(C)cc2C)c(F)c([C@@H](CC(=O)O)NC(=O)[C@@H](CC(C)C)n2cc(C3CCN(CC4CC4)CC3)c(C(F)(F)F)cc2=O)c1F] |
| 300 | 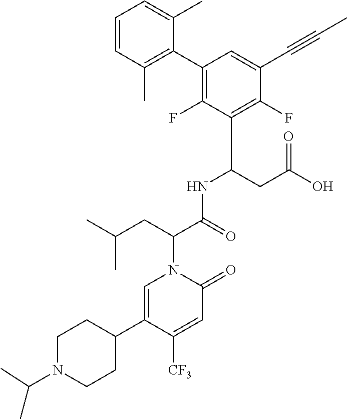 [SMILES: CC#Cc1cc(-c2c(C)cccc2C)c(F)c(C(CC(=O)O)NC(=O)C(CC(C)C)n2cc(C3CCN(C(C)C)CC3)c(C(F)(F)F)cc2=O)c1F] | 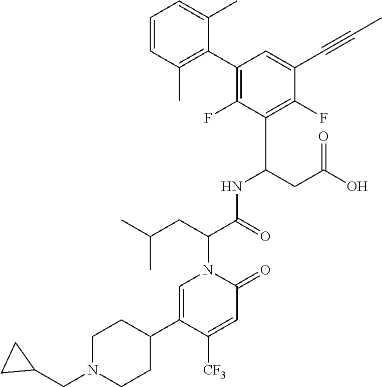 [SMILES: CC#Cc1cc(-c2c(C)cccc2C)c(F)c(C(CC(=O)O)NC(=O)C(CC(C)C)n2cc(C3CCN(CC4CC4)CC3)c(C(F)(F)F)cc2=O)c1F] |
| 301 | 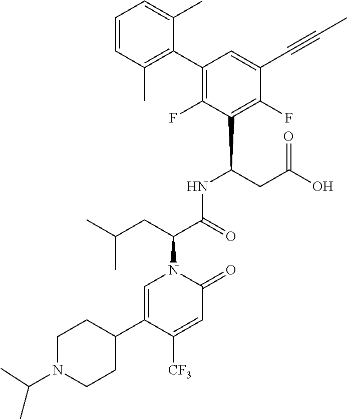 [SMILES: CC#Cc1cc(-c2c(C)cccc2C)c(F)c([C@@H](CC(=O)O)NC(=O)[C@H](CC(C)C)n2cc(C3CCN(C(C)C)CC3)c(C(F)(F)F)cc2=O)c1F] | 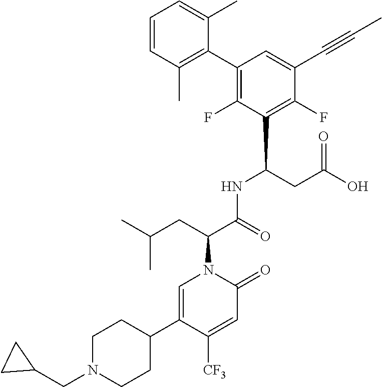 [SMILES: CC#Cc1cc(-c2c(C)cccc2C)c(F)c([C@@H](CC(=O)O)NC(=O)[C@H](CC(C)C)n2cc(C3CCN(CC4CC4)CC3)c(C(F)(F)F)cc2=O)c1F] |
| 302 | 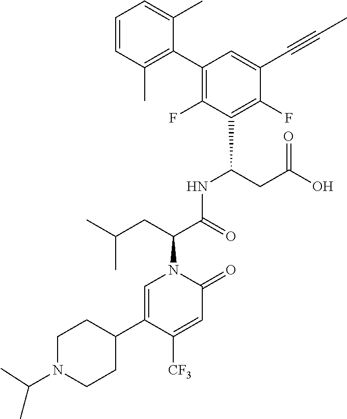 [SMILES: CC#Cc1cc(-c2c(C)cccc2C)c(F)c([C@H](CC(=O)O)NC(=O)[C@H](CC(C)C)n2cc(C3CCN(C(C)C)CC3)c(C(F)(F)F)cc2=O)c1F] | 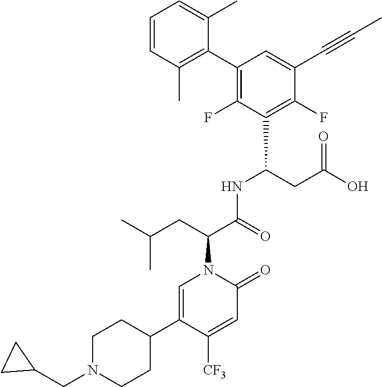 [SMILES: CC#Cc1cc(-c2c(C)cccc2C)c(F)c([C@H](CC(=O)O)NC(=O)[C@H](CC(C)C)n2cc(C3CCN(CC4CC4)CC3)c(C(F)(F)F)cc2=O)c1F] |
| 303 | 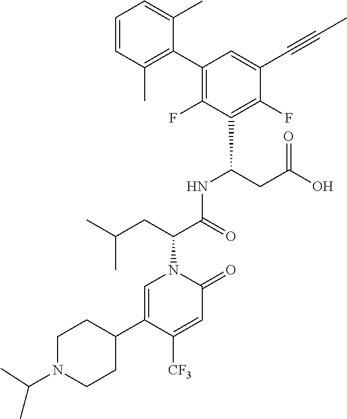 [SMILES: CC#Cc1cc(-c2c(C)cccc2C)c(F)c([C@H](CC(=O)O)NC(=O)[C@@H](CC(C)C)n2cc(C3CCN(C(C)C)CC3)c(C(F)(F)F)cc2=O)c1F] | 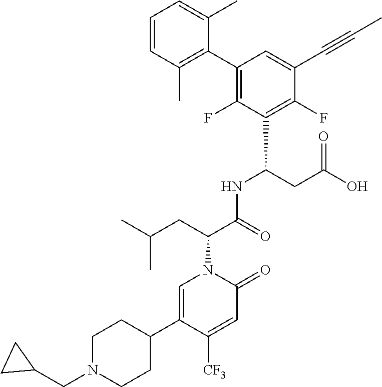 [SMILES: CC#Cc1cc(-c2c(C)cccc2C)c(F)c([C@H](CC(=O)O)NC(=O)[C@@H](CC(C)C)n2cc(C3CCN(CC4CC4)CC3)c(C(F)(F)F)cc2=O)c1F] |
| 304 | 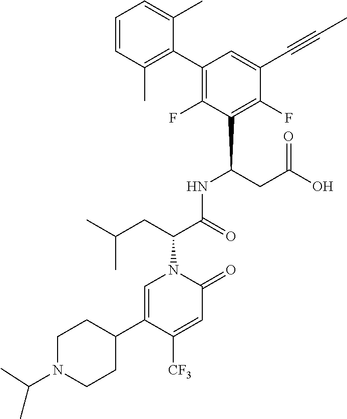 [SMILES: CC#Cc1cc(-c2c(C)cccc2C)c(F)c([C@@H](CC(=O)O)NC(=O)[C@@H](CC(C)C)n2cc(C3CCN(C(C)C)CC3)c(C(F)(F)F)cc2=O)c1F] | 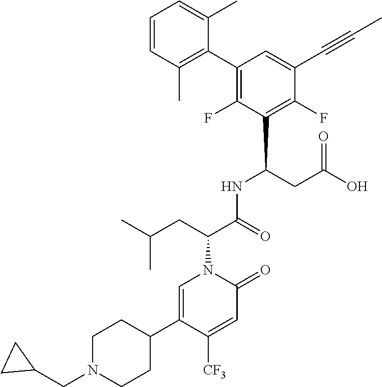 [SMILES: CC#Cc1cc(-c2c(C)cccc2C)c(F)c([C@@H](CC(=O)O)NC(=O)[C@@H](CC(C)C)n2cc(C3CCN(CC4CC4)CC3)c(C(F)(F)F)cc2=O)c1F] |
| 305 | 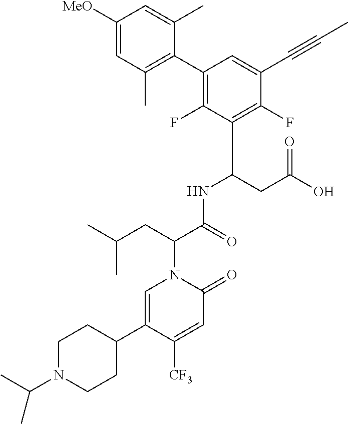 [SMILES: CC#Cc1cc(-c2c(C)cc(OC)cc2C)c(F)c(C(CC(=O)O)NC(=O)C(CC(C)C)n2cc(C3CCN(C(C)C)CC3)c(C(F)(F)F)cc2=O)c1F] | 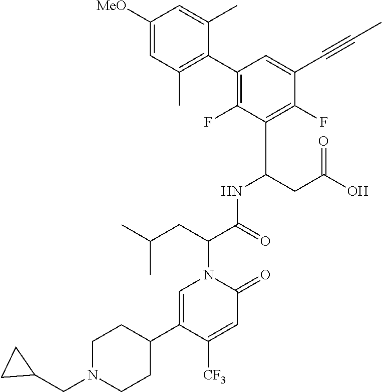 [SMILES: CC#Cc1cc(-c2c(C)cc(OC)cc2C)c(F)c(C(CC(=O)O)NC(=O)C(CC(C)C)n2cc(C3CCN(CC4CC4)CC3)c(C(F)(F)F)cc2=O)c1F] |
| 306 | 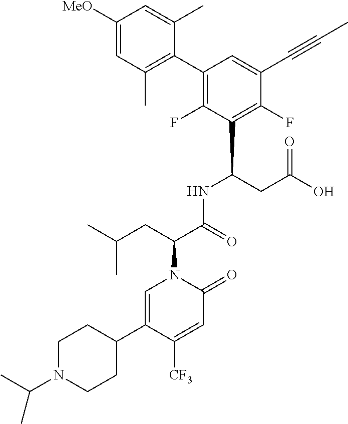 [SMILES: CC#Cc1cc(-c2c(C)cc(OC)cc2C)c(F)c([C@@H](CC(=O)O)NC(=O)[C@H](CC(C)C)n2cc(C3CCN(C(C)C)CC3)c(C(F)(F)F)cc2=O)c1F] | 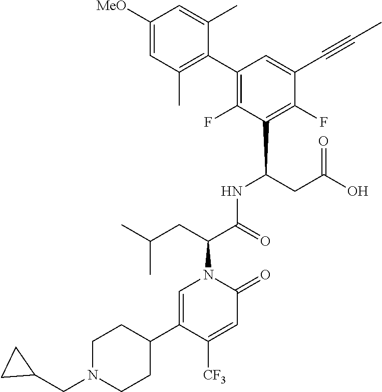 [SMILES: CC#Cc1cc(-c2c(C)cc(OC)cc2C)c(F)c([C@@H](CC(=O)O)NC(=O)[C@H](CC(C)C)n2cc(C3CCN(CC4CC4)CC3)c(C(F)(F)F)cc2=O)c1F] |
| 307 | 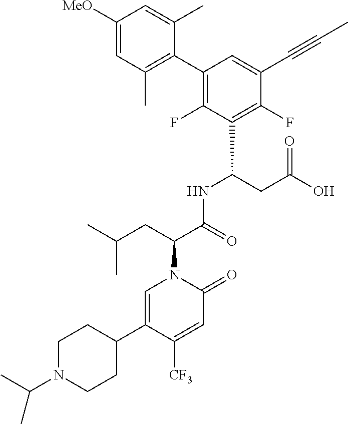 [SMILES: CC#Cc1cc(-c2c(C)cc(OC)cc2C)c(F)c([C@H](CC(=O)O)NC(=O)[C@H](CC(C)C)n2cc(C3CCN(C(C)C)CC3)c(C(F)(F)F)cc2=O)c1F] | 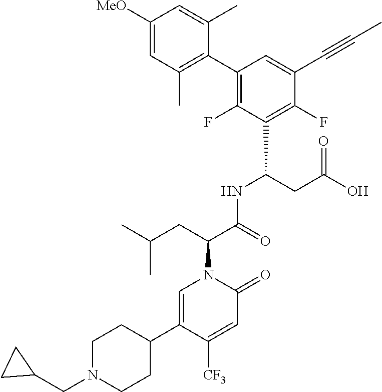 [SMILES: CC#Cc1cc(-c2c(C)cc(OC)cc2C)c(F)c([C@H](CC(=O)O)NC(=O)[C@H](CC(C)C)n2cc(C3CCN(CC4CC4)CC3)c(C(F)(F)F)cc2=O)c1F] |
| 308 | 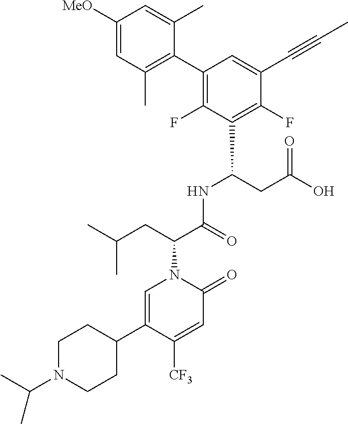 [SMILES: CC#Cc1cc(-c2c(C)cc(OC)cc2C)c(F)c([C@H](CC(=O)O)NC(=O)[C@@H](CC(C)C)n2cc(C3CCN(C(C)C)CC3)c(C(F)(F)F)cc2=O)c1F] | 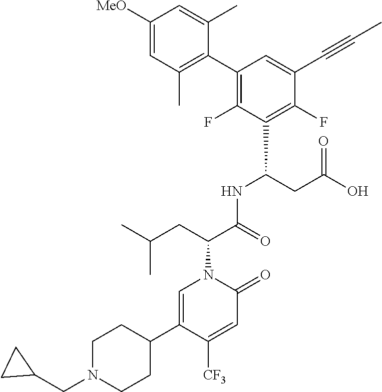 [SMILES: CC#Cc1cc(-c2c(C)cc(OC)cc2C)c(F)c([C@H](CC(=O)O)NC(=O)[C@@H](CC(C)C)n2cc(C3CCN(CC4CC4)CC3)c(C(F)(F)F)cc2=O)c1F] |
| 309 | 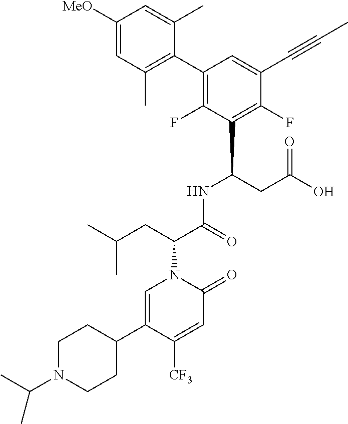 [SMILES: CC#Cc1cc(-c2c(C)cc(OC)cc2C)c(F)c([C@@H](CC(=O)O)NC(=O)[C@@H](CC(C)C)n2cc(C3CCN(C(C)C)CC3)c(C(F)(F)F)cc2=O)c1F] | 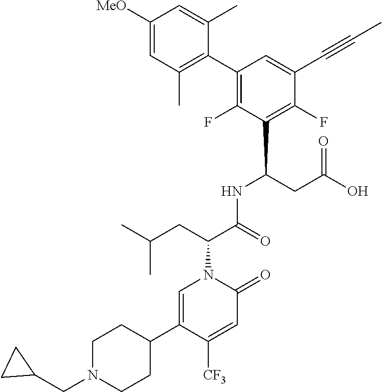 [SMILES: CC#Cc1cc(-c2c(C)cc(OC)cc2C)c(F)c([C@@H](CC(=O)O)NC(=O)[C@@H](CC(C)C)n2cc(C3CCN(CC4CC4)CC3)c(C(F)(F)F)cc2=O)c1F] |
| 310 | 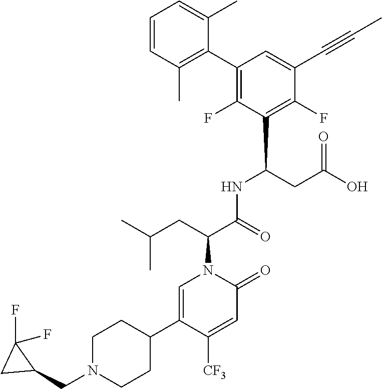 [SMILES: CC#Cc1cc(-c2c(C)cccc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] | 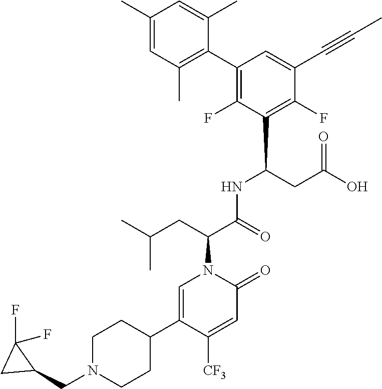 [SMILES: CC#Cc1cc(-c2c(C)cc(C)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] |
| 311 | 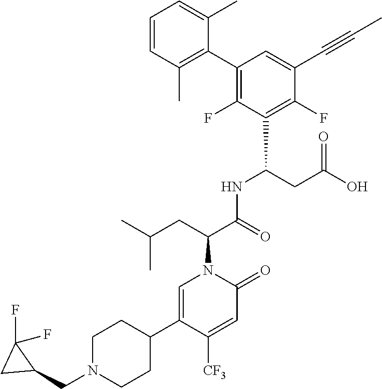 [SMILES: CC#Cc1cc(-c2c(C)cccc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] | 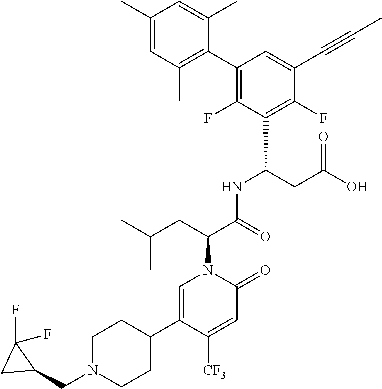 [SMILES: CC#Cc1cc(-c2c(C)cc(C)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 312 | 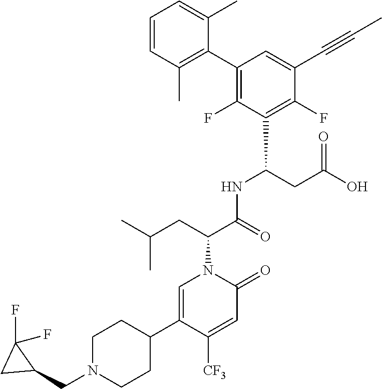 [SMILES: CC#Cc1cc(-c2c(C)cccc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] | 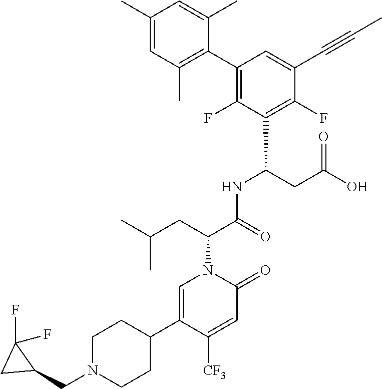 [SMILES: CC#Cc1cc(-c2c(C)cc(C)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] |
| 313 | 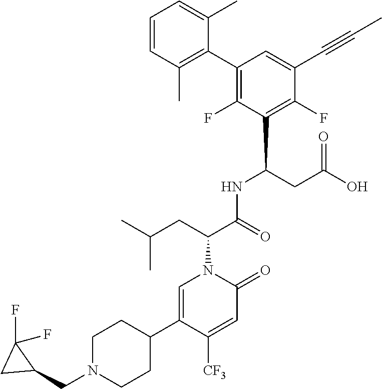 [SMILES: CC#Cc1cc(-c2c(C)cccc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] | 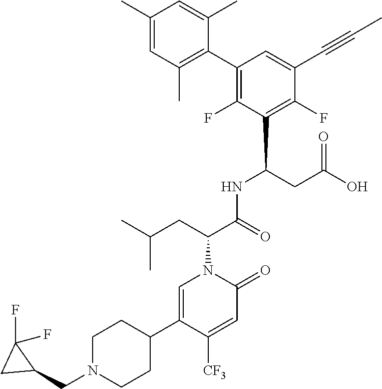 [SMILES: CC#Cc1cc(-c2c(C)cc(C)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] |
| 314 | 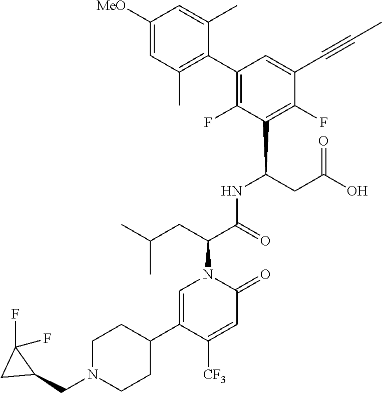 [SMILES: CC#Cc1cc(-c2c(C)cc(OC)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] | 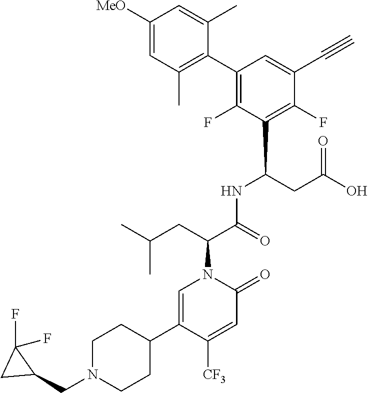 [SMILES: C#Cc1cc(-c2c(C)cc(OC)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] |
| 315 | 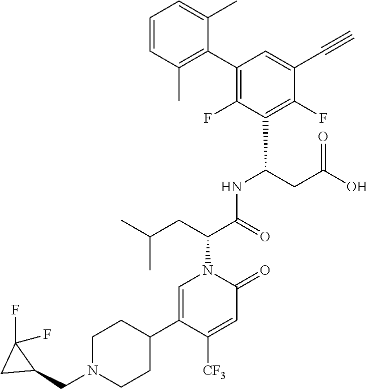 [SMILES: C#Cc1cc(-c2c(C)cccc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] | 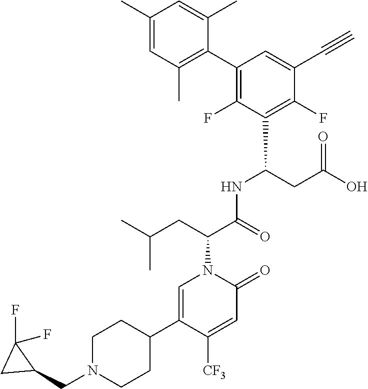 [SMILES: C#Cc1cc(-c2c(C)cc(C)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] |
| 316 | 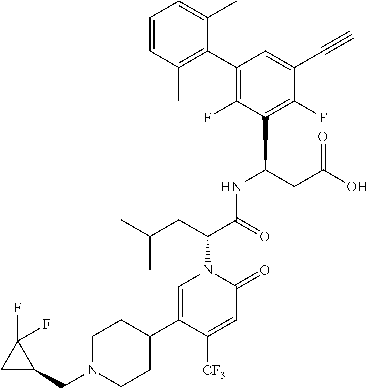 [SMILES: C#Cc1cc(-c2c(C)cccc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] | 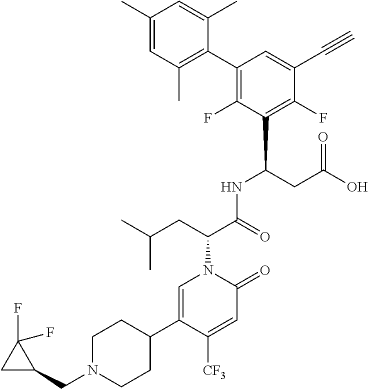 [SMILES: C#Cc1cc(-c2c(C)cc(C)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] |
| 317 | 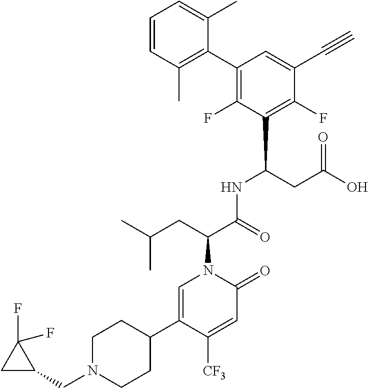 [SMILES: C#Cc1cc(-c2c(C)cccc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] | 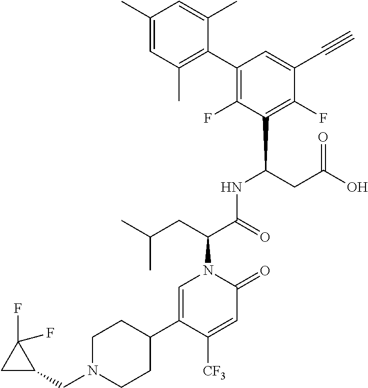 [SMILES: C#Cc1cc(-c2c(C)cc(C)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] |
| 318 | 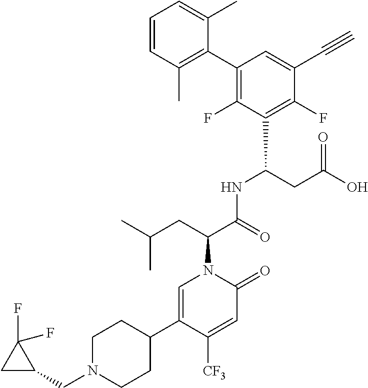 [SMILES: C#Cc1cc(-c2c(C)cccc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] | 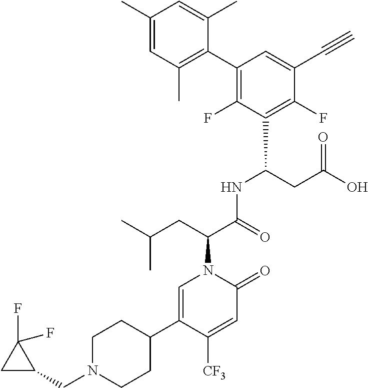 [SMILES: C#Cc1cc(-c2c(C)cc(C)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 319 | 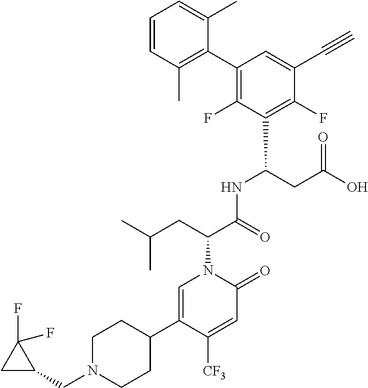 [SMILES: C#Cc1cc(-c2c(C)cccc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] | 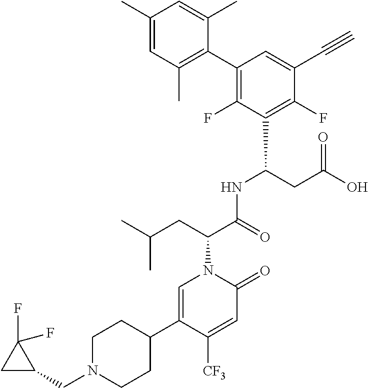 [SMILES: C#Cc1cc(-c2c(C)cc(C)cc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] |
| 320 | 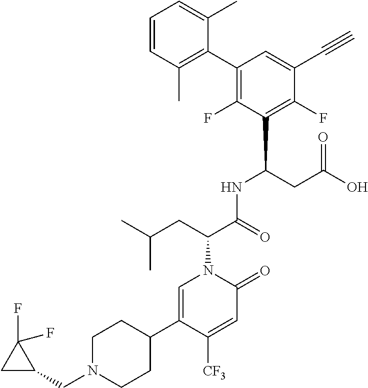 [SMILES: C#Cc1cc(-c2c(C)cccc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] | 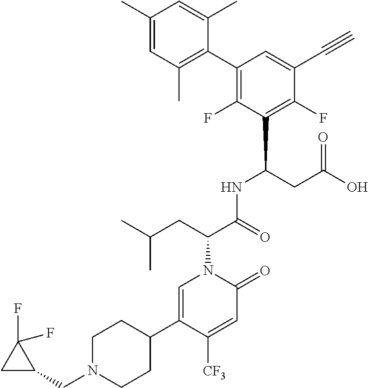 [SMILES: C#Cc1cc(-c2c(C)cc(C)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] |
| 321 | 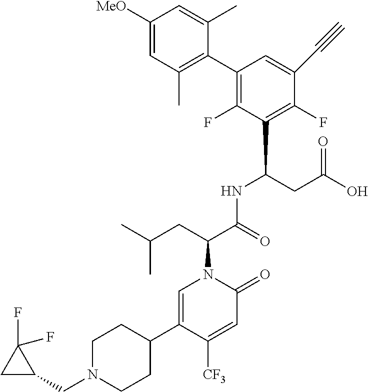 [SMILES: C#Cc1cc(-c2c(C)cc(OC)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] | 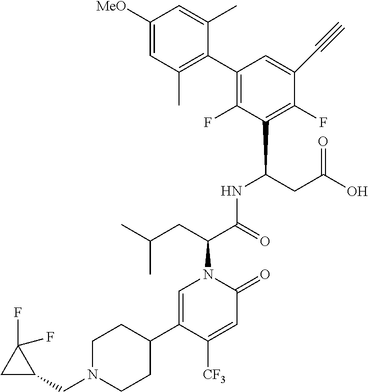 [SMILES: C#Cc1cc(-c2c(C)cc(OC)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] |
| 322 | 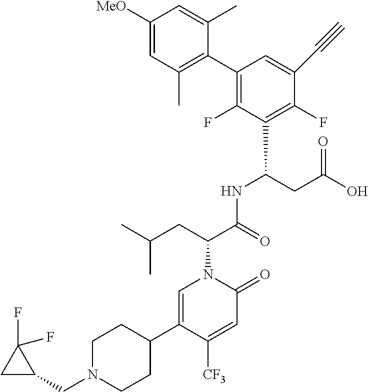 [SMILES: C#Cc1cc(-c2c(C)cc(OC)cc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] | 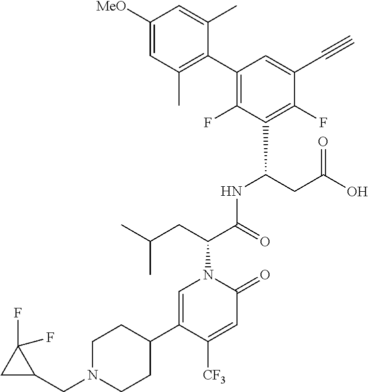 [SMILES: C#Cc1cc(-c2c(C)cc(OC)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] |
| 323 | 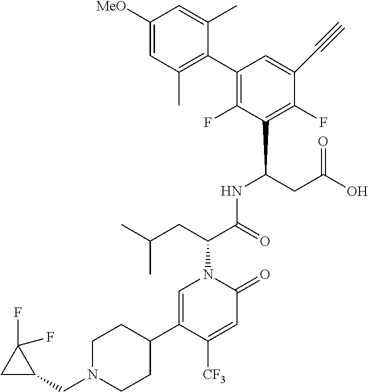 [SMILES: C#Cc1cc(-c2c(C)cc(OC)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] | 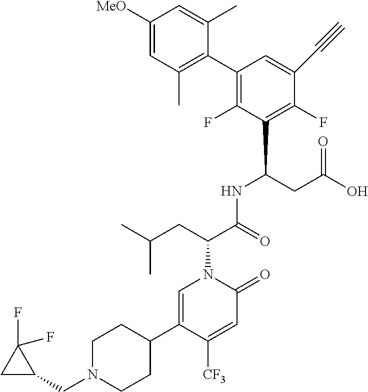 [SMILES: C#Cc1cc(-c2c(C)cc(OC)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] |
| 324 | 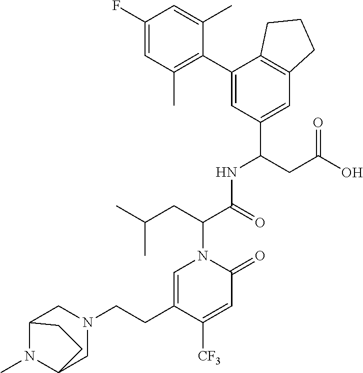 [SMILES: Cc1cc(F)cc(C)c1-c1cc(C(CC(=O)O)NC(=O)C(CC(C)C)n2cc(CCN3CC4CCC(C3)N4C)c(C(F)(F)F)cc2=O)cc2c1CCC2] | 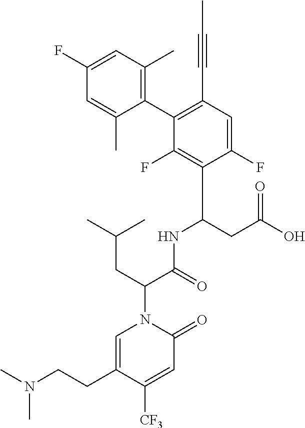 [SMILES: CC#Cc1cc(F)c(C(CC(=O)O)NC(=O)C(CC(C)C)n2cc(CCN(C)C)c(C(F)(F)F)cc2=O)c(F)c1-c1c(C)cc(F)cc1C] |
| 325 | 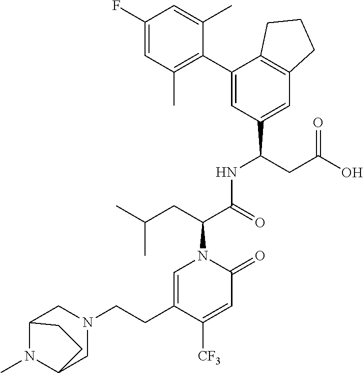 [SMILES: Cc1cc(F)cc(C)c1-c1cc([C@@H](CC(=O)O)NC(=O)[C@H](CC(C)C)n2cc(CCN3CC4CCC(C3)N4C)c(C(F)(F)F)cc2=O)cc2c1CCC2] | 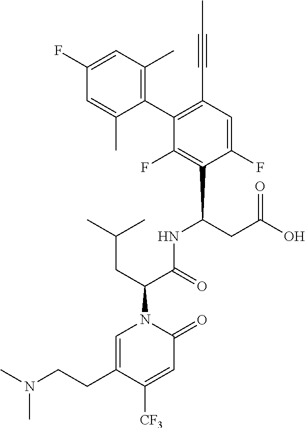 [SMILES: CC#Cc1cc(F)c([C@@H](CC(=O)O)NC(=O)[C@H](CC(C)C)n2cc(CCN(C)C)c(C(F)(F)F)cc2=O)c(F)c1-c1c(C)cc(F)cc1C] |
| 326 | 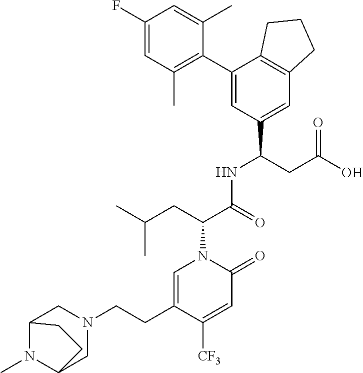 [SMILES: Cc1cc(F)cc(C)c1-c1cc([C@@H](CC(=O)O)NC(=O)[C@@H](CC(C)C)n2cc(CCN3CC4CCC(C3)N4C)c(C(F)(F)F)cc2=O)cc2c1CCC2] | 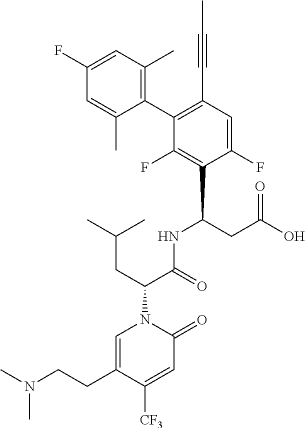 [SMILES: CC#Cc1cc(F)c([C@@H](CC(=O)O)NC(=O)[C@@H](CC(C)C)n2cc(CCN(C)C)c(C(F)(F)F)cc2=O)c(F)c1-c1c(C)cc(F)cc1C] |
| 327 | 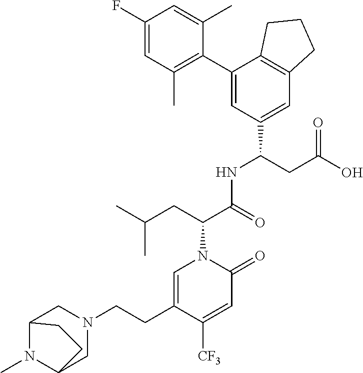 [SMILES: Cc1cc(F)cc(C)c1-c1cc([C@H](CC(=O)O)NC(=O)[C@@H](CC(C)C)n2cc(CCN3CC4CCC(C3)N4C)c(C(F)(F)F)cc2=O)cc2c1CCC2] | 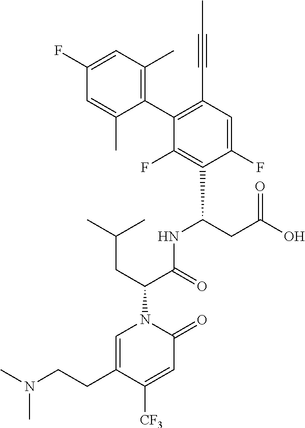 [SMILES: CC#Cc1cc(F)c([C@H](CC(=O)O)NC(=O)[C@@H](CC(C)C)n2cc(CCN(C)C)c(C(F)(F)F)cc2=O)c(F)c1-c1c(C)cc(F)cc1C] |
| 328 | 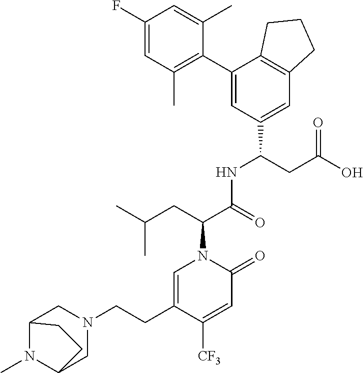 [SMILES: Cc1cc(F)cc(C)c1-c1cc([C@H](CC(=O)O)NC(=O)[C@H](CC(C)C)n2cc(CCN3CC4CCC(C3)N4C)c(C(F)(F)F)cc2=O)cc2c1CCC2] | 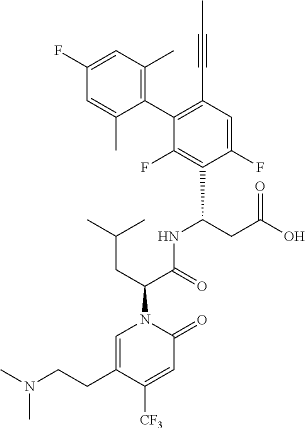 [SMILES: CC#Cc1cc(F)c([C@H](CC(=O)O)NC(=O)[C@H](CC(C)C)n2cc(CCN(C)C)c(C(F)(F)F)cc2=O)c(F)c1-c1c(C)cc(F)cc1C] |
| 329 | 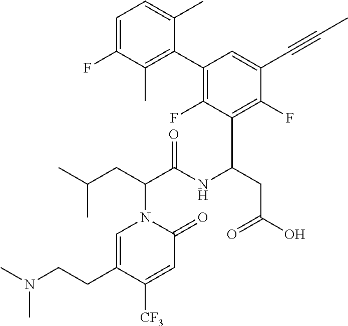 [SMILES: CC#Cc1cc(-c2c(C)ccc(F)c2C)c(F)c(C(CC(=O)O)NC(=O)C(CC(C)C)n2cc(CCN(C)C)c(C(F)(F)F)cc2=O)c1F] | 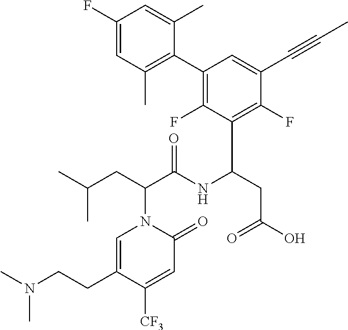 [SMILES: CC#Cc1cc(-c2c(C)cc(F)cc2C)c(F)c(C(CC(=O)O)NC(=O)C(CC(C)C)n2cc(CCN(C)C)c(C(F)(F)F)cc2=O)c1F] |
| 330 | 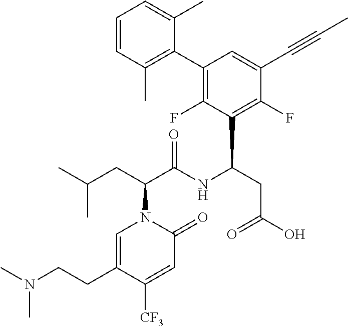 [SMILES: CC#Cc1cc(-c2c(C)cccc2C)c(F)c([C@@H](CC(=O)O)NC(=O)[C@H](CC(C)C)n2cc(CCN(C)C)c(C(F)(F)F)cc2=O)c1F] | 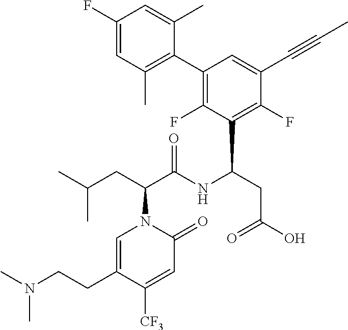 [SMILES: CC#Cc1cc(-c2c(C)cc(F)cc2C)c(F)c([C@@H](CC(=O)O)NC(=O)[C@H](CC(C)C)n2cc(CCN(C)C)c(C(F)(F)F)cc2=O)c1F] |
| 331 | 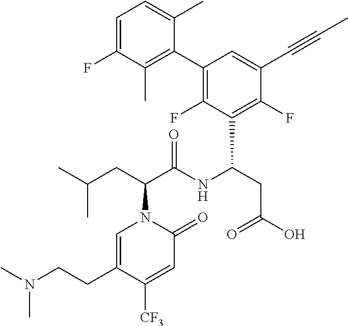 [SMILES: CC#Cc1cc(-c2c(C)ccc(F)c2C)c(F)c([C@H](CC(=O)O)NC(=O)[C@H](CC(C)C)n2cc(CCN(C)C)c(C(F)(F)F)cc2=O)c1F] | 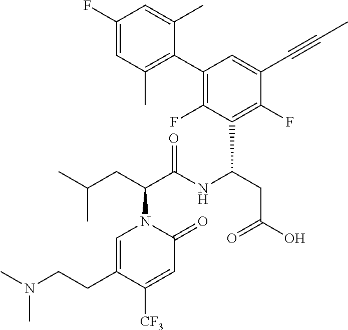 [SMILES: CC#Cc1cc(-c2c(C)cc(F)cc2C)c(F)c([C@H](CC(=O)O)NC(=O)[C@H](CC(C)C)n2cc(CCN(C)C)c(C(F)(F)F)cc2=O)c1F] |
| 332 | 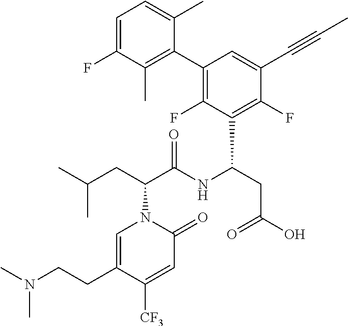 [SMILES: CC#Cc1cc(-c2c(C)ccc(F)c2C)c(F)c([C@H](CC(=O)O)NC(=O)[C@@H](CC(C)C)n2cc(CCN(C)C)c(C(F)(F)F)cc2=O)c1F] | 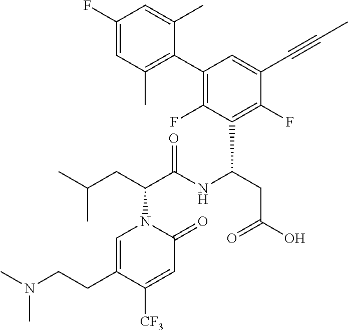 [SMILES: CC#Cc1cc(-c2c(C)cc(F)cc2C)c(F)c([C@H](CC(=O)O)NC(=O)[C@@H](CC(C)C)n2cc(CCN(C)C)c(C(F)(F)F)cc2=O)c1F] |
| 333 | 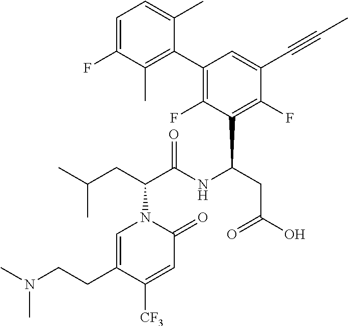 [SMILES: CC#Cc1cc(-c2c(C)ccc(F)c2C)c(F)c([C@@H](CC(=O)O)NC(=O)[C@@H](CC(C)C)n2cc(CCN(C)C)c(C(F)(F)F)cc2=O)c1F] | 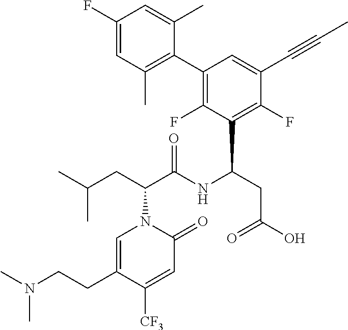 [SMILES: CC#Cc1cc(-c2c(C)cc(F)cc2C)c(F)c([C@@H](CC(=O)O)NC(=O)[C@@H](CC(C)C)n2cc(CCN(C)C)c(C(F)(F)F)cc2=O)c1F] |
| 334 | 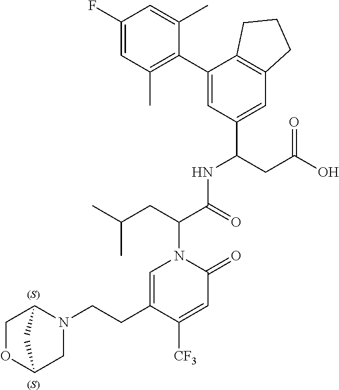 | 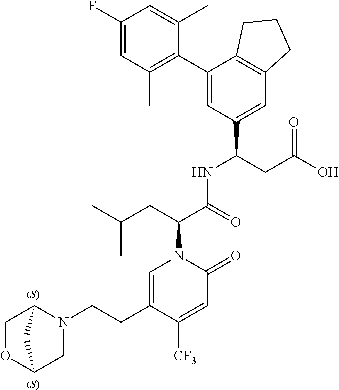 |
| 335 | 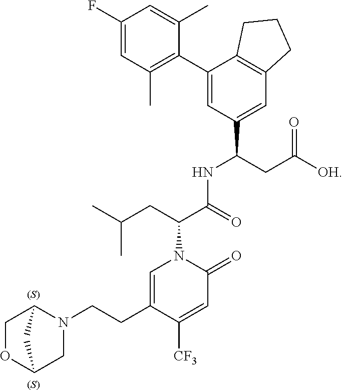 | |

| | | |
|---|---|---|
| 110 | 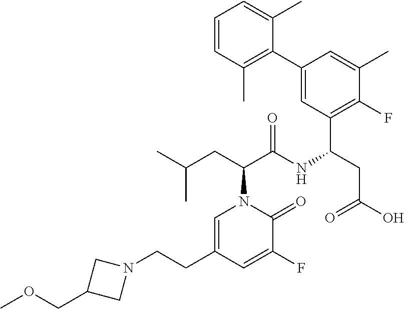 [SMILES: COCC1CN(CCc2cc(F)c(=O)n([C@@H](CC(C)C)C(=O)N[C@@H](CC(=O)O)c3cc(-c4c(C)cccc4C)cc(C)c3F)c2)C1] | 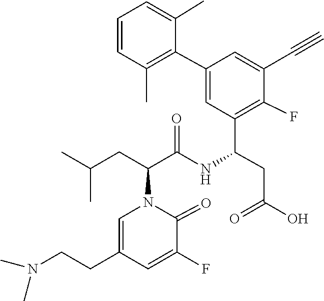 [SMILES: C#Cc1cc(-c2c(C)cccc2C)cc([C@H](CC(=O)O)NC(=O)[C@H](CC(C)C)n2cc(CCN(C)C)cc(F)c2=O)c1F] |
| 111 | 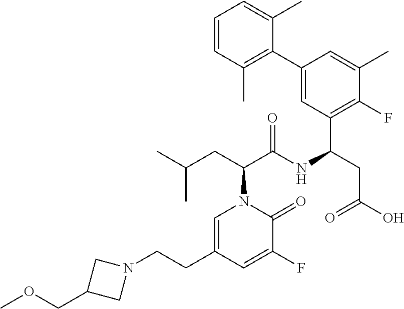 [SMILES: COCC1CN(CCc2cc(F)c(=O)n([C@@H](CC(C)C)C(=O)N[C@H](CC(=O)O)c3cc(-c4c(C)cccc4C)cc(C)c3F)c2)C1] | 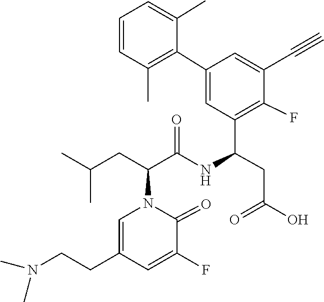 [SMILES: C#Cc1cc(-c2c(C)cccc2C)cc([C@@H](CC(=O)O)NC(=O)[C@H](CC(C)C)n2cc(CCN(C)C)cc(F)c2=O)c1F] |
| 112 | 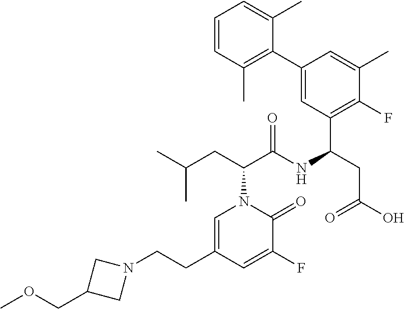 [SMILES: COCC1CN(CCc2cc(F)c(=O)n([C@H](CC(C)C)C(=O)N[C@H](CC(=O)O)c3cc(-c4c(C)cccc4C)cc(C)c3F)c2)C1] | 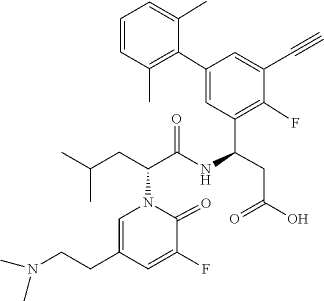 [SMILES: C#Cc1cc(-c2c(C)cccc2C)cc([C@@H](CC(=O)O)NC(=O)[C@@H](CC(C)C)n2cc(CCN(C)C)cc(F)c2=O)c1F] |
| 113 | 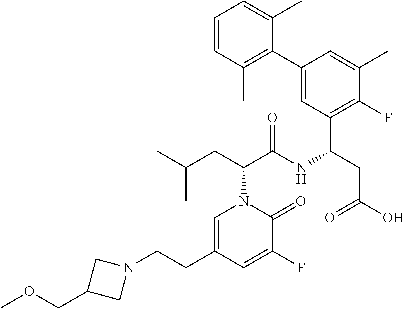 [SMILES: COCC1CN(CCc2cc(F)c(=O)n([C@H](CC(C)C)C(=O)N[C@@H](CC(=O)O)c3cc(-c4c(C)cccc4C)cc(C)c3F)c2)C1] | 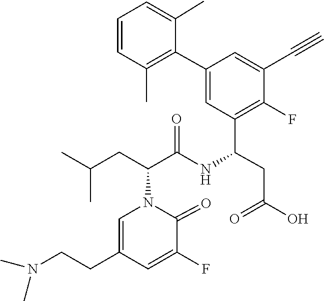 [SMILES: C#Cc1cc(-c2c(C)cccc2C)cc([C@H](CC(=O)O)NC(=O)[C@@H](CC(C)C)n2cc(CCN(C)C)cc(F)c2=O)c1F] |
| 114 | 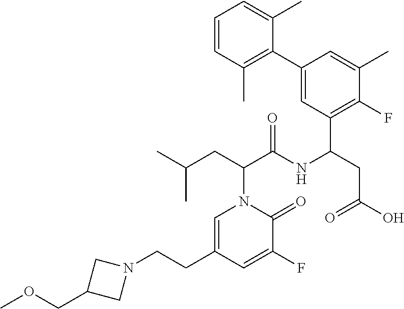 [SMILES: COCC1CN(CCc2cc(F)c(=O)n(C(CC(C)C)C(=O)NC(CC(=O)O)c3cc(-c4c(C)cccc4C)cc(C)c3F)c2)C1] | 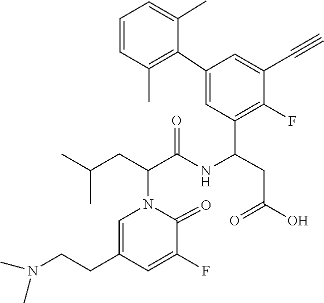 [SMILES: C#Cc1cc(-c2c(C)cccc2C)cc(C(CC(=O)O)NC(=O)C(CC(C)C)n2cc(CCN(C)C)cc(F)c2=O)c1F] |
| 115 | 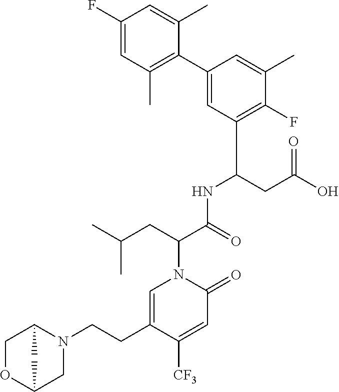 [SMILES: Cc1cc(-c2c(C)cc(F)cc2C)cc(C(CC(=O)O)NC(=O)C(CC(C)C)n2cc(CCN3C[C@@H]4C[C@H]3CO4)c(C(F)(F)F)cc2=O)c1F] | 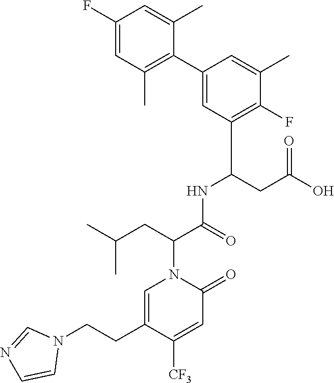 [SMILES: Cc1cc(-c2c(C)cc(F)cc2C)cc(C(CC(=O)O)NC(=O)C(CC(C)C)n2cc(CCn3ccnc3)c(C(F)(F)F)cc2=O)c1F] |
| 116 | 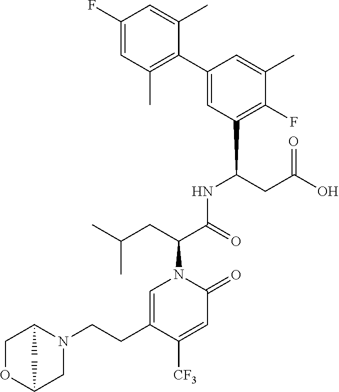 [SMILES: Cc1cc(-c2c(C)cc(F)cc2C)cc([C@@H](CC(=O)O)NC(=O)[C@H](CC(C)C)n2cc(CCN3C[C@@H]4C[C@H]3CO4)c(C(F)(F)F)cc2=O)c1F] | 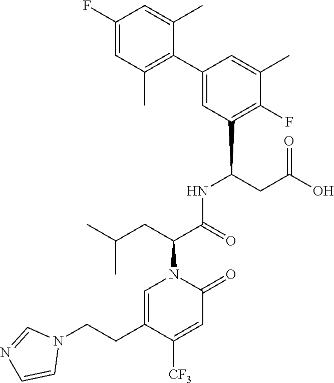 [SMILES: Cc1cc(-c2c(C)cc(F)cc2C)cc([C@@H](CC(=O)O)NC(=O)[C@H](CC(C)C)n2cc(CCn3ccnc3)c(C(F)(F)F)cc2=O)c1F] |
| 117 | 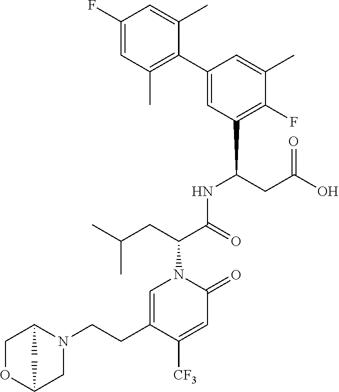 [SMILES: Cc1cc(-c2c(C)cc(F)cc2C)cc([C@@H](CC(=O)O)NC(=O)[C@@H](CC(C)C)n2cc(CCN3C[C@@H]4C[C@H]3CO4)c(C(F)(F)F)cc2=O)c1F] | 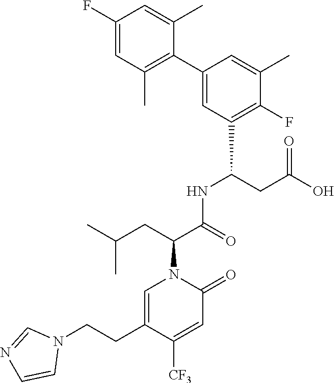 [SMILES: Cc1cc(-c2c(C)cc(F)cc2C)cc([C@H](CC(=O)O)NC(=O)[C@H](CC(C)C)n2cc(CCn3ccnc3)c(C(F)(F)F)cc2=O)c1F] |
| 118 | 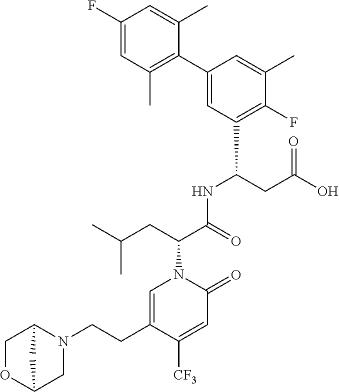 [SMILES: Cc1cc(-c2c(C)cc(F)cc2C)cc([C@H](CC(=O)O)NC(=O)[C@@H](CC(C)C)n2cc(CCN3C[C@@H]4C[C@H]3CO4)c(C(F)(F)F)cc2=O)c1F] | 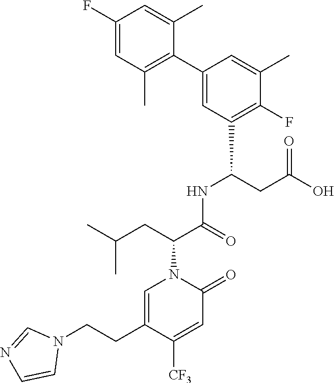 [SMILES: Cc1cc(-c2c(C)cc(F)cc2C)cc([C@H](CC(=O)O)NC(=O)[C@@H](CC(C)C)n2cc(CCn3ccnc3)c(C(F)(F)F)cc2=O)c1F] |
| 119 | 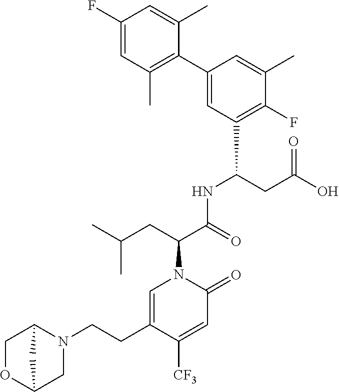 [SMILES: Cc1cc(-c2c(C)cc(F)cc2C)cc([C@H](CC(=O)O)NC(=O)[C@H](CC(C)C)n2cc(CCN3C[C@@H]4C[C@H]3CO4)c(C(F)(F)F)cc2=O)c1F] | 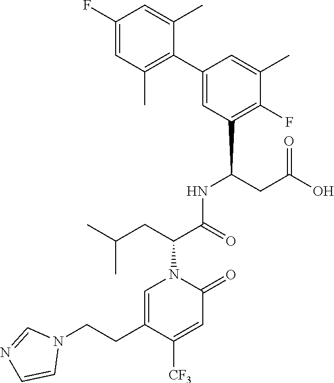 [SMILES: Cc1cc(-c2c(C)cc(F)cc2C)cc([C@@H](CC(=O)O)NC(=O)[C@@H](CC(C)C)n2cc(CCn3ccnc3)c(C(F)(F)F)cc2=O)c1F] |
| 120 | 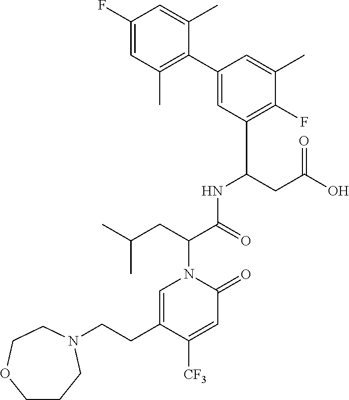 [SMILES: Cc1cc(-c2c(C)cc(F)cc2C)cc(C(CC(=O)O)NC(=O)C(CC(C)C)n2cc(CCN3CCCOCC3)c(C(F)(F)F)cc2=O)c1F] | 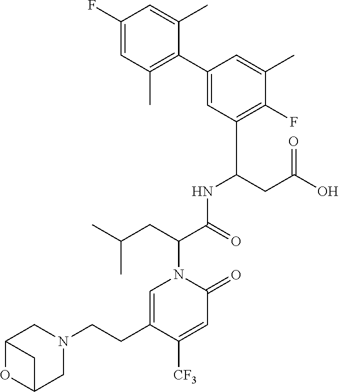 [SMILES: Cc1cc(-c2c(C)cc(F)cc2C)cc(C(CC(=O)O)NC(=O)C(CC(C)C)n2cc(CCN3CC4CC(C3)O4)c(C(F)(F)F)cc2=O)c1F] |
| 121 | 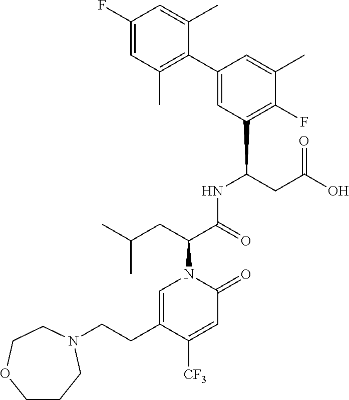 [SMILES: Cc1cc(-c2c(C)cc(F)cc2C)cc([C@@H](CC(=O)O)NC(=O)[C@H](CC(C)C)n2cc(CCN3CCCOCC3)c(C(F)(F)F)cc2=O)c1F] | 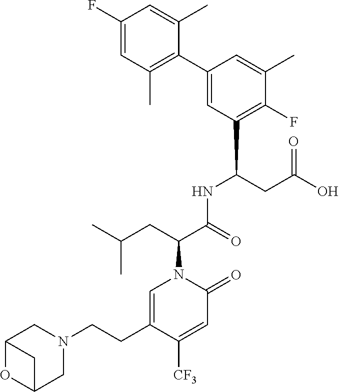 [SMILES: Cc1cc(-c2c(C)cc(F)cc2C)cc([C@@H](CC(=O)O)NC(=O)[C@H](CC(C)C)n2cc(CCN3CC4CC(C3)O4)c(C(F)(F)F)cc2=O)c1F] |
| 122 | 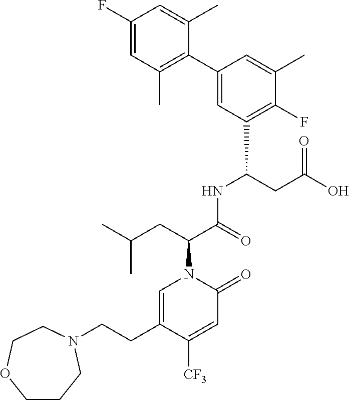 [SMILES: Cc1cc(-c2c(C)cc(F)cc2C)cc([C@H](CC(=O)O)NC(=O)[C@H](CC(C)C)n2cc(CCN3CCCOCC3)c(C(F)(F)F)cc2=O)c1F] | 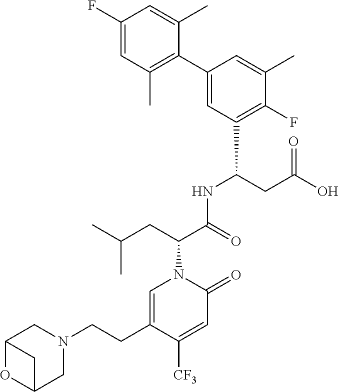 [SMILES: Cc1cc(-c2c(C)cc(F)cc2C)cc([C@H](CC(=O)O)NC(=O)[C@@H](CC(C)C)n2cc(CCN3CC4CC(C3)O4)c(C(F)(F)F)cc2=O)c1F] |
| 123 | 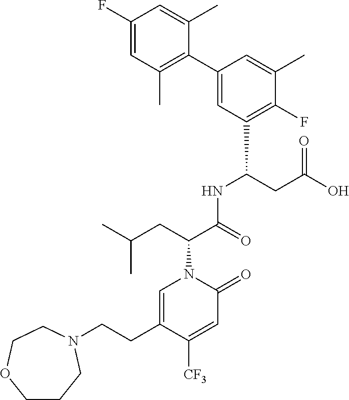 [SMILES: Cc1cc(-c2c(C)cc(F)cc2C)cc([C@H](CC(=O)O)NC(=O)[C@@H](CC(C)C)n2cc(CCN3CCCOCC3)c(C(F)(F)F)cc2=O)c1F] | 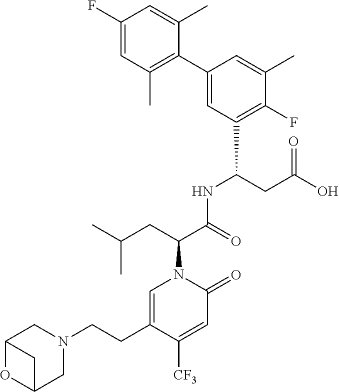 [SMILES: Cc1cc(-c2c(C)cc(F)cc2C)cc([C@H](CC(=O)O)NC(=O)[C@H](CC(C)C)n2cc(CCN3CC4CC(C3)O4)c(C(F)(F)F)cc2=O)c1F] |
| 124 | 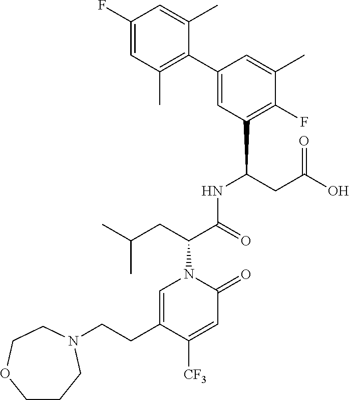 [SMILES: Cc1cc(-c2c(C)cc(F)cc2C)cc([C@@H](CC(=O)O)NC(=O)[C@@H](CC(C)C)n2cc(CCN3CCCOCC3)c(C(F)(F)F)cc2=O)c1F] | 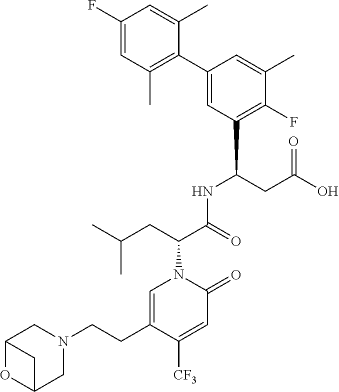 [SMILES: Cc1cc(-c2c(C)cc(F)cc2C)cc([C@@H](CC(=O)O)NC(=O)[C@@H](CC(C)C)n2cc(CCN3CC4CC(C3)O4)c(C(F)(F)F)cc2=O)c1F] |
| 125 | 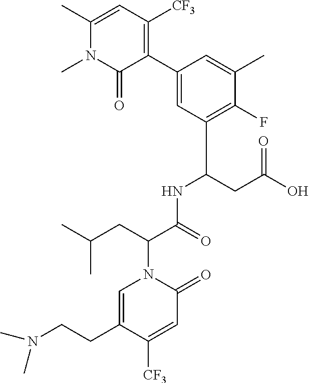 [SMILES: Cc1cc(-c2c(C(F)(F)F)cc(C)n(C)c2=O)cc(C(CC(=O)O)NC(=O)C(CC(C)C)n2cc(CCN(C)C)c(C(F)(F)F)cc2=O)c1F] | 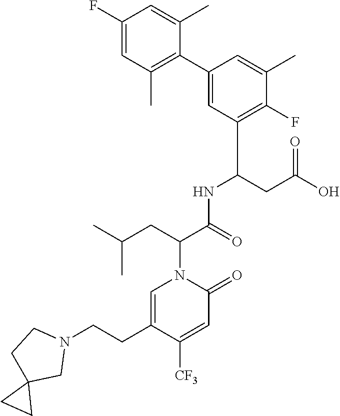 [SMILES: Cc1cc(-c2c(C)cc(F)cc2C)cc(C(CC(=O)O)NC(=O)C(CC(C)C)n2cc(CCN3CCC4(CC4)C3)c(C(F)(F)F)cc2=O)c1F] |
| 126 | 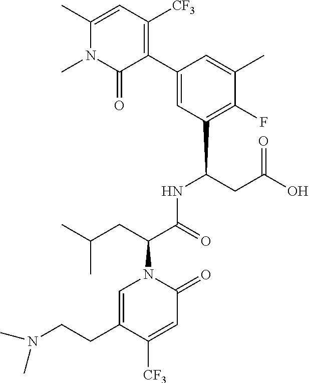 [SMILES: Cc1cc(-c2c(C(F)(F)F)cc(C)n(C)c2=O)cc([C@@H](CC(=O)O)NC(=O)[C@H](CC(C)C)n2cc(CCN(C)C)c(C(F)(F)F)cc2=O)c1F] | 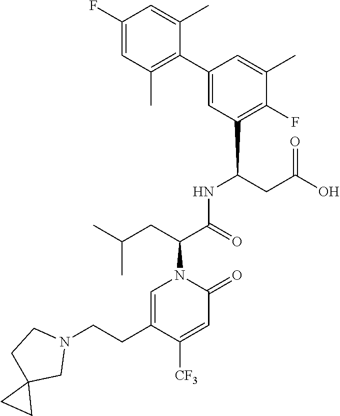 [SMILES: Cc1cc(-c2c(C)cc(F)cc2C)cc([C@@H](CC(=O)O)NC(=O)[C@H](CC(C)C)n2cc(CCN3CCC4(CC4)C3)c(C(F)(F)F)cc2=O)c1F] |
| 127 | 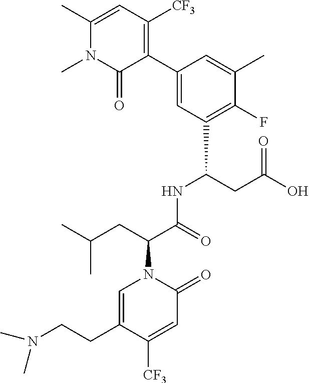 [SMILES: Cc1cc(-c2c(C(F)(F)F)cc(C)n(C)c2=O)cc([C@H](CC(=O)O)NC(=O)[C@H](CC(C)C)n2cc(CCN(C)C)c(C(F)(F)F)cc2=O)c1F] | 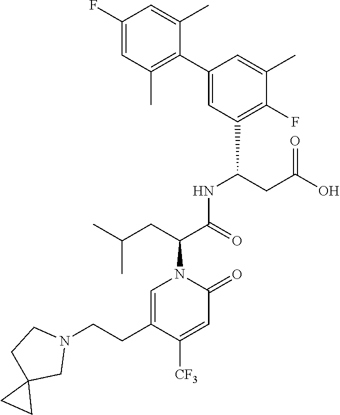 [SMILES: Cc1cc(-c2c(C)cc(F)cc2C)cc([C@H](CC(=O)O)NC(=O)[C@H](CC(C)C)n2cc(CCN3CCC4(CC4)C3)c(C(F)(F)F)cc2=O)c1F] |
| 128 | 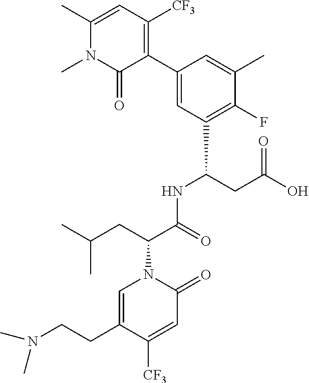 [SMILES: Cc1cc(-c2c(C(F)(F)F)cc(C)n(C)c2=O)cc([C@H](CC(=O)O)NC(=O)[C@@H](CC(C)C)n2cc(CCN(C)C)c(C(F)(F)F)cc2=O)c1F] | 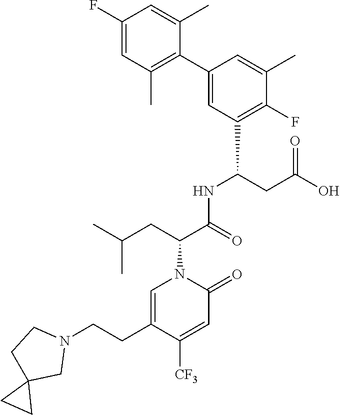 [SMILES: Cc1cc(-c2c(C)cc(F)cc2C)cc([C@H](CC(=O)O)NC(=O)[C@@H](CC(C)C)n2cc(CCN3CCC4(CC4)C3)c(C(F)(F)F)cc2=O)c1F] |
| 129 | 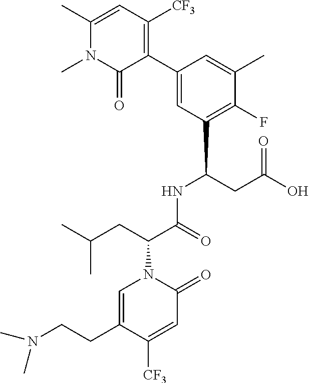 [SMILES: Cc1cc(-c2c(C(F)(F)F)cc(C)n(C)c2=O)cc([C@@H](CC(=O)O)NC(=O)[C@@H](CC(C)C)n2cc(CCN(C)C)c(C(F)(F)F)cc2=O)c1F] | 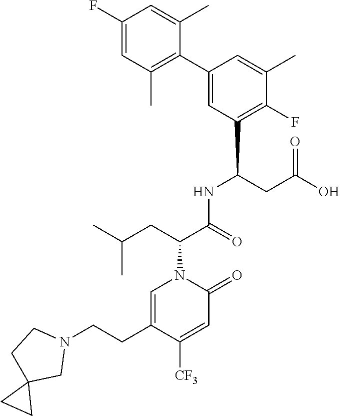 [SMILES: Cc1cc(-c2c(C)cc(F)cc2C)cc([C@@H](CC(=O)O)NC(=O)[C@@H](CC(C)C)n2cc(CCN3CCC4(CC4)C3)c(C(F)(F)F)cc2=O)c1F] |
| 130 | 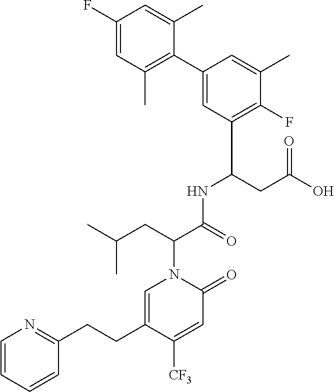 [SMILES: Cc1cc(-c2c(C)cc(F)cc2C)cc(C(CC(=O)O)NC(=O)C(CC(C)C)n2cc(CCc3ccccn3)c(C(F)(F)F)cc2=O)c1F] | 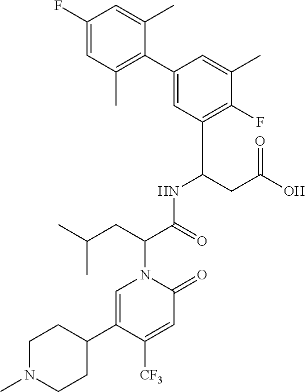 [SMILES: Cc1cc(-c2c(C)cc(F)cc2C)cc(C(CC(=O)O)NC(=O)C(CC(C)C)n2cc(C3CCN(C)CC3)c(C(F)(F)F)cc2=O)c1F] |
| 131 | 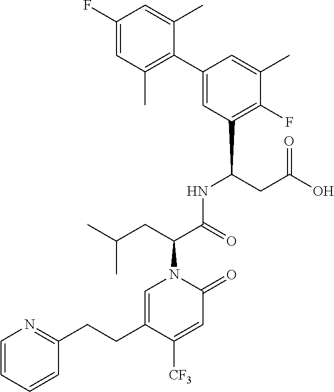 [SMILES: Cc1cc(-c2c(C)cc(F)cc2C)cc([C@@H](CC(=O)O)NC(=O)[C@H](CC(C)C)n2cc(CCc3ccccn3)c(C(F)(F)F)cc2=O)c1F] | 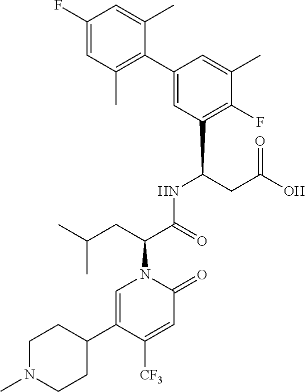 [SMILES: Cc1cc(-c2c(C)cc(F)cc2C)cc([C@@H](CC(=O)O)NC(=O)[C@H](CC(C)C)n2cc(C3CCN(C)CC3)c(C(F)(F)F)cc2=O)c1F] |
| 132 | 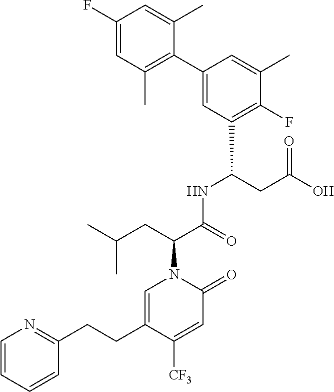 [SMILES: Cc1cc(-c2c(C)cc(F)cc2C)cc([C@H](CC(=O)O)NC(=O)[C@H](CC(C)C)n2cc(CCc3ccccn3)c(C(F)(F)F)cc2=O)c1F] | 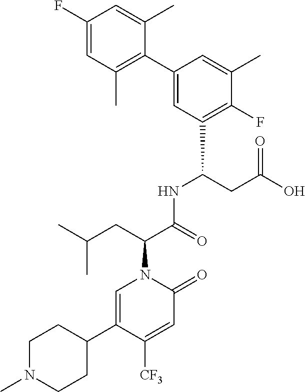 [SMILES: Cc1cc(-c2c(C)cc(F)cc2C)cc([C@H](CC(=O)O)NC(=O)[C@H](CC(C)C)n2cc(C3CCN(C)CC3)c(C(F)(F)F)cc2=O)c1F] |
| 133 | 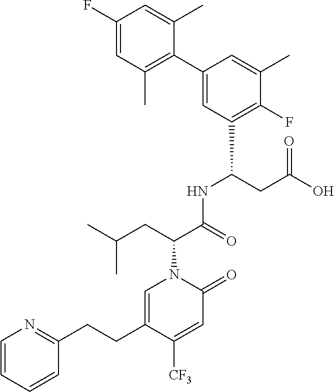 [SMILES: Cc1cc(-c2c(C)cc(F)cc2C)cc([C@H](CC(=O)O)NC(=O)[C@@H](CC(C)C)n2cc(CCc3ccccn3)c(C(F)(F)F)cc2=O)c1F] | 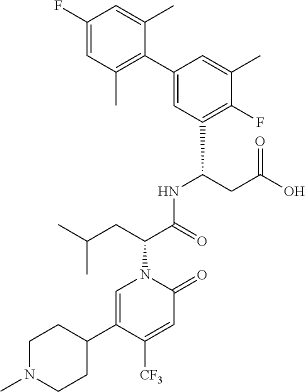 [SMILES: Cc1cc(-c2c(C)cc(F)cc2C)cc([C@H](CC(=O)O)NC(=O)[C@@H](CC(C)C)n2cc(C3CCN(C)CC3)c(C(F)(F)F)cc2=O)c1F] |
| 134 | 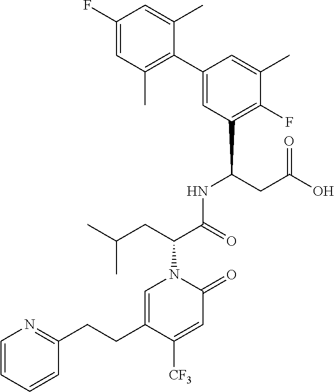 [SMILES: Cc1cc(-c2c(C)cc(F)cc2C)cc([C@@H](CC(=O)O)NC(=O)[C@@H](CC(C)C)n2cc(CCc3ccccn3)c(C(F)(F)F)cc2=O)c1F] | 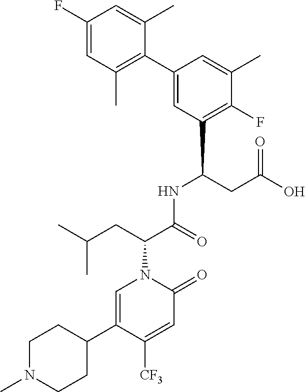 [SMILES: Cc1cc(-c2c(C)cc(F)cc2C)cc([C@@H](CC(=O)O)NC(=O)[C@@H](CC(C)C)n2cc(C3CCN(C)CC3)c(C(F)(F)F)cc2=O)c1F] |
| 135 | 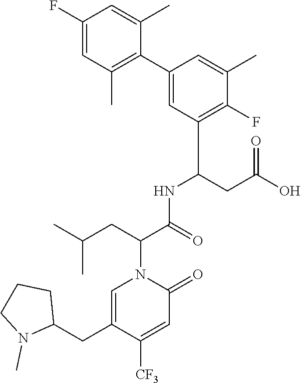 [SMILES: Cc1cc(-c2c(C)cc(F)cc2C)cc(C(CC(=O)O)NC(=O)C(CC(C)C)n2cc(CC3CCCN3C)c(C(F)(F)F)cc2=O)c1F] | 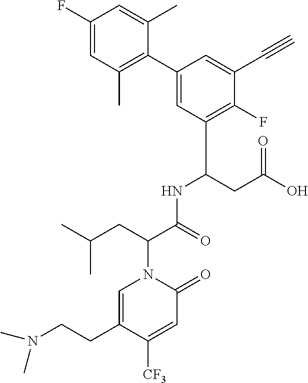 [SMILES: C#Cc1cc(-c2c(C)cc(F)cc2C)cc(C(CC(=O)O)NC(=O)C(CC(C)C)n2cc(CCN(C)C)c(C(F)(F)F)cc2=O)c1F] |
| 136 | 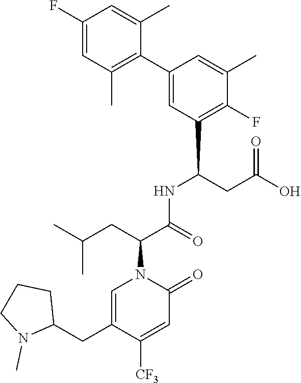 [SMILES: Cc1cc(-c2c(C)cc(F)cc2C)cc([C@@H](CC(=O)O)NC(=O)[C@H](CC(C)C)n2cc(CC3CCCN3C)c(C(F)(F)F)cc2=O)c1F] | 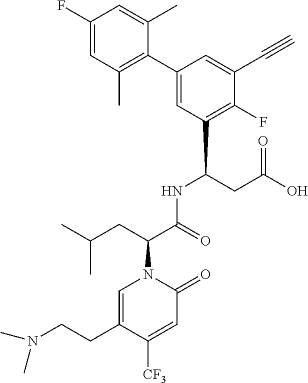 [SMILES: C#Cc1cc(-c2c(C)cc(F)cc2C)cc([C@@H](CC(=O)O)NC(=O)[C@H](CC(C)C)n2cc(CCN(C)C)c(C(F)(F)F)cc2=O)c1F] |
| 137 | 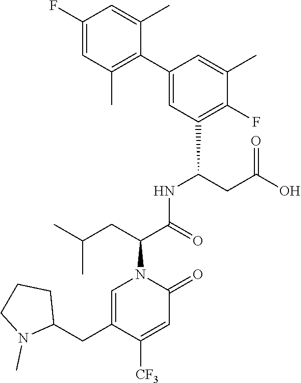 [SMILES: Cc1cc(-c2c(C)cc(F)cc2C)cc([C@H](CC(=O)O)NC(=O)[C@H](CC(C)C)n2cc(CC3CCCN3C)c(C(F)(F)F)cc2=O)c1F] | 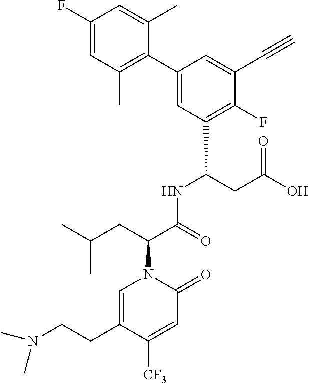 [SMILES: C#Cc1cc(-c2c(C)cc(F)cc2C)cc([C@H](CC(=O)O)NC(=O)[C@H](CC(C)C)n2cc(CCN(C)C)c(C(F)(F)F)cc2=O)c1F] |
| 138 | 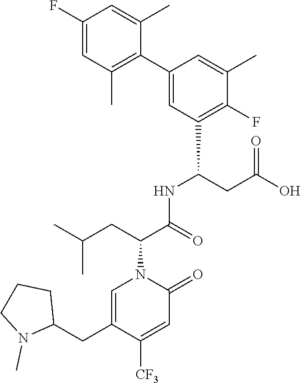 [SMILES: Cc1cc(-c2c(C)cc(F)cc2C)cc([C@H](CC(=O)O)NC(=O)[C@@H](CC(C)C)n2cc(CC3CCCN3C)c(C(F)(F)F)cc2=O)c1F] | 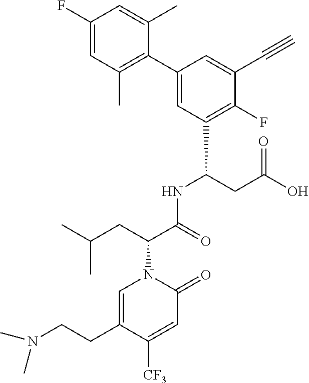 [SMILES: C#Cc1cc(-c2c(C)cc(F)cc2C)cc([C@H](CC(=O)O)NC(=O)[C@@H](CC(C)C)n2cc(CCN(C)C)c(C(F)(F)F)cc2=O)c1F] |
| 139 | 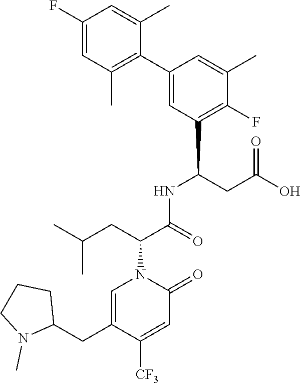 [SMILES: Cc1cc(-c2c(C)cc(F)cc2C)cc([C@@H](CC(=O)O)NC(=O)[C@@H](CC(C)C)n2cc(CC3CCCN3C)c(C(F)(F)F)cc2=O)c1F] | 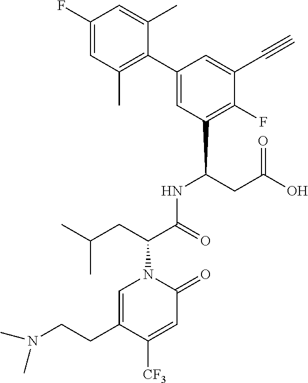 [SMILES: C#Cc1cc(-c2c(C)cc(F)cc2C)cc([C@@H](CC(=O)O)NC(=O)[C@@H](CC(C)C)n2cc(CCN(C)C)c(C(F)(F)F)cc2=O)c1F] |
| 140 | 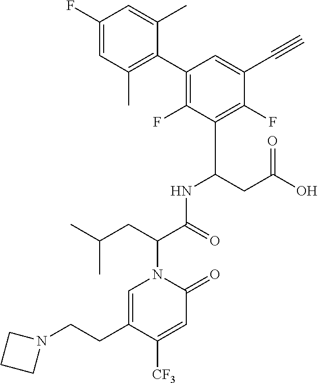 [SMILES: C#Cc1cc(-c2c(C)cc(F)cc2C)c(F)c(C(CC(=O)O)NC(=O)C(CC(C)C)n2cc(CCN3CCC3)c(C(F)(F)F)cc2=O)c1F] | 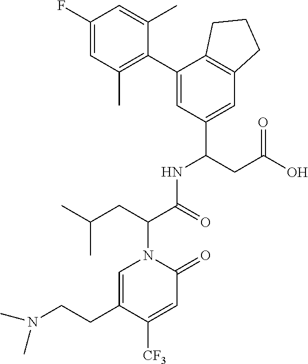 [SMILES: Cc1cc(F)cc(C)c1-c1cc(C(CC(=O)O)NC(=O)C(CC(C)C)n2cc(CCN(C)C)c(C(F)(F)F)cc2=O)cc2c1CCC2] |
| 141 | 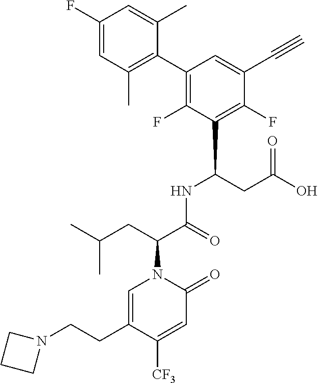 [SMILES: C#Cc1cc(-c2c(C)cc(F)cc2C)c(F)c([C@@H](CC(=O)O)NC(=O)[C@H](CC(C)C)n2cc(CCN3CCC3)c(C(F)(F)F)cc2=O)c1F] | 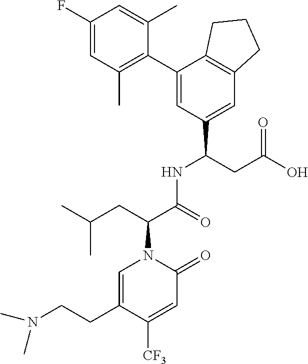 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)cc2c1CCC2] |
| 142 | 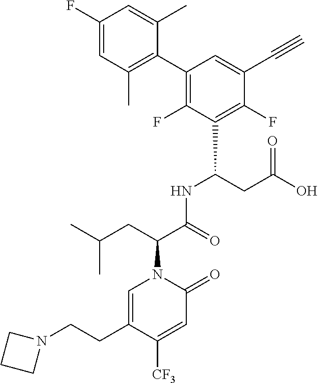 [SMILES: C#Cc1cc(-c2c(C)cc(F)cc2C)c(F)c([C@H](CC(=O)O)NC(=O)[C@H](CC(C)C)n2cc(CCN3CCC3)c(C(F)(F)F)cc2=O)c1F] | 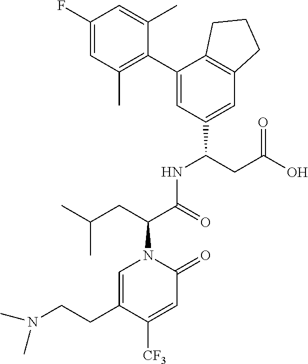 [SMILES: Cc1cc(F)cc(C)c1-c1cc([C@H](CC(=O)O)NC(=O)[C@H](CC(C)C)n2cc(CCN(C)C)c(C(F)(F)F)cc2=O)cc2c1CCC2] |
| 143 | 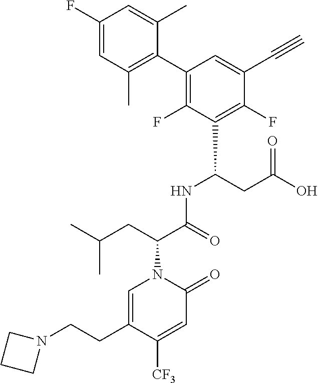 [SMILES: C#Cc1cc(-c2c(C)cc(F)cc2C)c(F)c([C@H](CC(=O)O)NC(=O)[C@@H](CC(C)C)n2cc(CCN3CCC3)c(C(F)(F)F)cc2=O)c1F] | 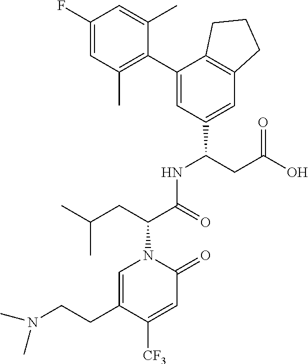 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cc2c1CCC2] |
| 144 | 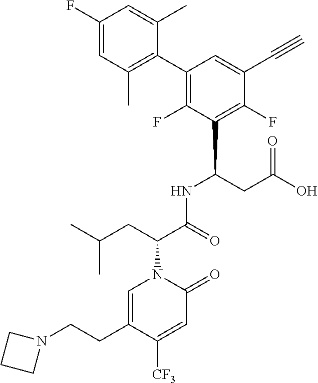 [SMILES: C#Cc1cc(-c2c(C)cc(F)cc2C)c(F)c([C@@H](CC(=O)O)NC(=O)[C@@H](CC(C)C)n2cc(CCN3CCC3)c(C(F)(F)F)cc2=O)c1F] | 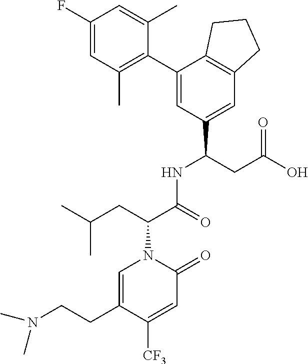 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)cc2c1CCC2] |
| 145 | 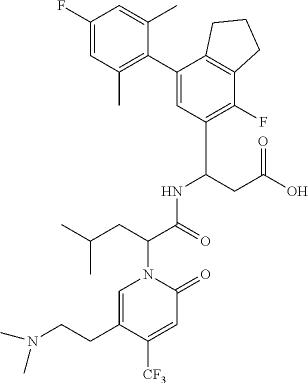 [SMILES: Cc1cc(F)cc(C)c1-c1cc(C(CC(=O)O)NC(=O)C(CC(C)C)n2cc(CCN(C)C)c(C(F)(F)F)cc2=O)c(F)c2c1CCC2] | |
| 146 | 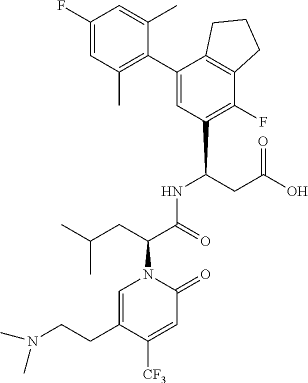 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)c(F)c2c1CCC2] | |
| 147 | 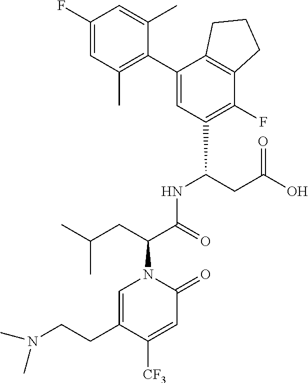 [SMILES: Cc1cc(F)cc(C)c1-c1cc([C@H](CC(=O)O)NC(=O)[C@H](CC(C)C)n2cc(CCN(C)C)c(C(F)(F)F)cc2=O)c(F)c2c1CCC2] | |
| 148 | 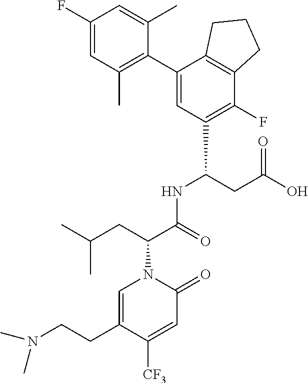 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)c(F)c2c1CCC2] | |
| 149 | 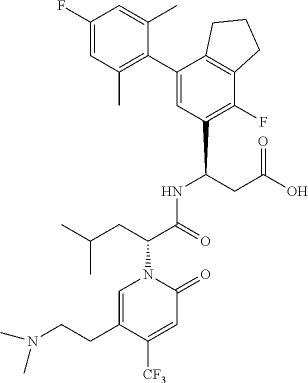 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)c(F)c2c1CCC2] | |
| 150 | 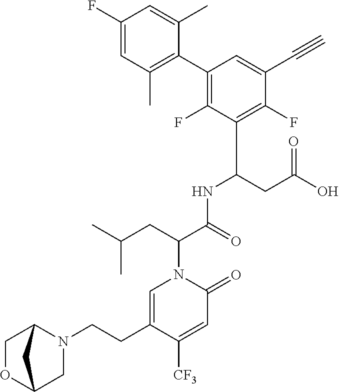 [SMILES: C#Cc1cc(-c2c(C)cc(F)cc2C)c(F)c(C(CC(=O)O)NC(=O)C(CC(C)C)n2cc(CCN3C[C@H]4C[C@@H]3CO4)c(C(F)(F)F)cc2=O)c1F] | 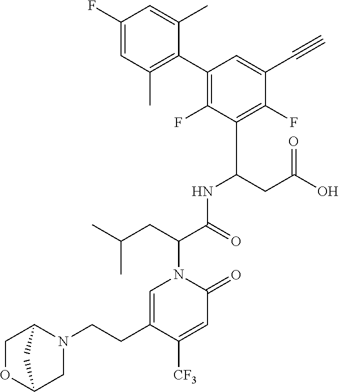 [SMILES: C#Cc1cc(-c2c(C)cc(F)cc2C)c(F)c(C(CC(=O)O)NC(=O)C(CC(C)C)n2cc(CCN3C[C@@H]4C[C@H]3CO4)c(C(F)(F)F)cc2=O)c1F] |
| 151 | 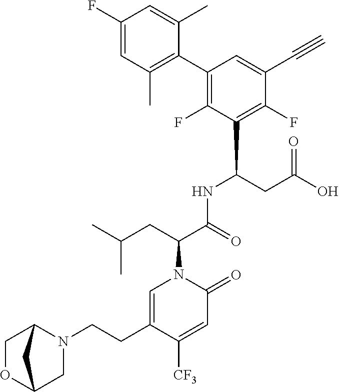 [SMILES: C#Cc1cc(-c2c(C)cc(F)cc2C)c(F)c([C@@H](CC(=O)O)NC(=O)[C@H](CC(C)C)n2cc(CCN3C[C@H]4C[C@@H]3CO4)c(C(F)(F)F)cc2=O)c1F] | 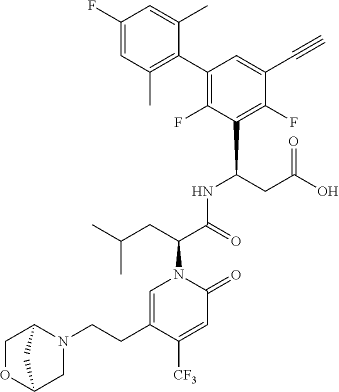 [SMILES: C#Cc1cc(-c2c(C)cc(F)cc2C)c(F)c([C@@H](CC(=O)O)NC(=O)[C@H](CC(C)C)n2cc(CCN3C[C@@H]4C[C@H]3CO4)c(C(F)(F)F)cc2=O)c1F] |
| 152 | 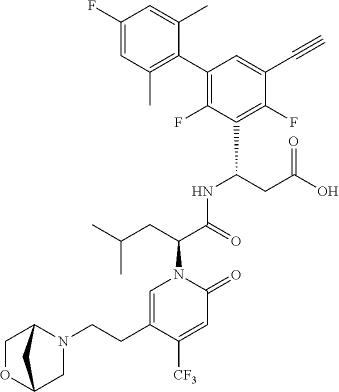 [SMILES: C#Cc1cc(-c2c(C)cc(F)cc2C)c(F)c([C@H](CC(=O)O)NC(=O)[C@H](CC(C)C)n2cc(CCN3C[C@H]4C[C@@H]3CO4)c(C(F)(F)F)cc2=O)c1F] | 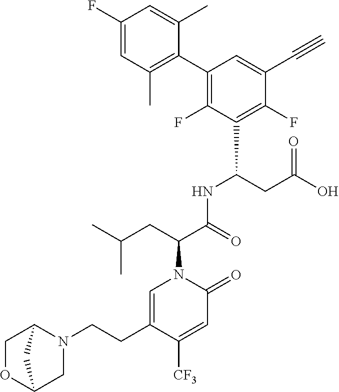 [SMILES: C#Cc1cc(-c2c(C)cc(F)cc2C)c(F)c([C@H](CC(=O)O)NC(=O)[C@H](CC(C)C)n2cc(CCN3C[C@@H]4C[C@H]3CO4)c(C(F)(F)F)cc2=O)c1F] |
| 153 | 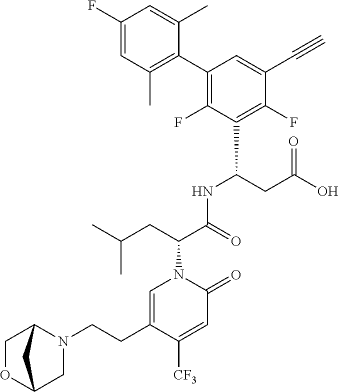 [SMILES: C#Cc1cc(-c2c(C)cc(F)cc2C)c(F)c([C@H](CC(=O)O)NC(=O)[C@@H](CC(C)C)n2cc(CCN3C[C@H]4C[C@@H]3CO4)c(C(F)(F)F)cc2=O)c1F] | 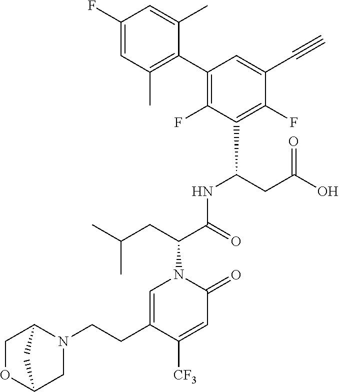 [SMILES: C#Cc1cc(-c2c(C)cc(F)cc2C)c(F)c([C@H](CC(=O)O)NC(=O)[C@@H](CC(C)C)n2cc(CCN3C[C@@H]4C[C@H]3CO4)c(C(F)(F)F)cc2=O)c1F] |
| 154 | 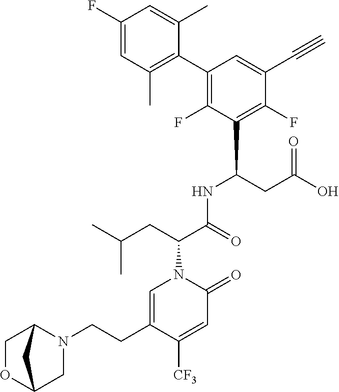 [SMILES: C#Cc1cc(-c2c(C)cc(F)cc2C)c(F)c([C@@H](CC(=O)O)NC(=O)[C@@H](CC(C)C)n2cc(CCN3C[C@H]4C[C@@H]3CO4)c(C(F)(F)F)cc2=O)c1F] | 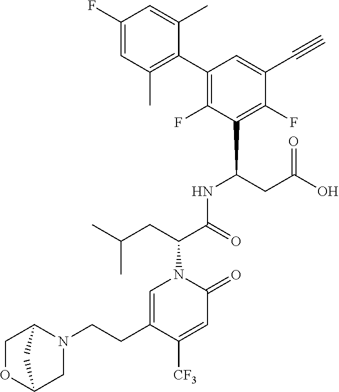 [SMILES: C#Cc1cc(-c2c(C)cc(F)cc2C)c(F)c([C@@H](CC(=O)O)NC(=O)[C@@H](CC(C)C)n2cc(CCN3C[C@@H]4C[C@H]3CO4)c(C(F)(F)F)cc2=O)c1F] |
| 155 | 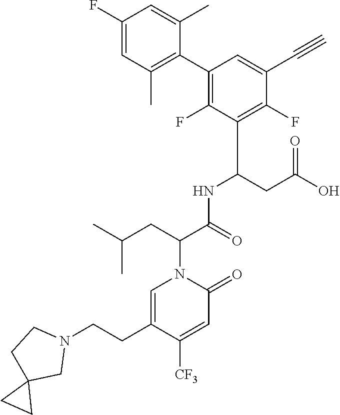 [SMILES: C#Cc1cc(-c2c(C)cc(F)cc2C)c(F)c(C(CC(=O)O)NC(=O)C(CC(C)C)n2cc(CCN3CCC4(CC4)C3)c(C(F)(F)F)cc2=O)c1F] | 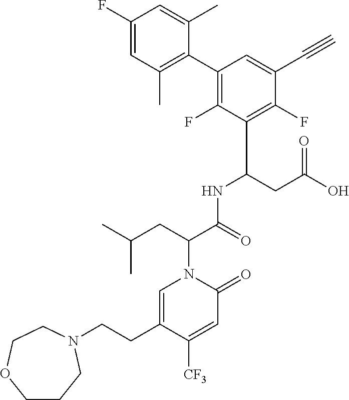 [SMILES: C#Cc1cc(-c2c(C)cc(F)cc2C)c(F)c(C(CC(=O)O)NC(=O)C(CC(C)C)n2cc(CCN3CCCOCC3)c(C(F)(F)F)cc2=O)c1F] |
| 156 | 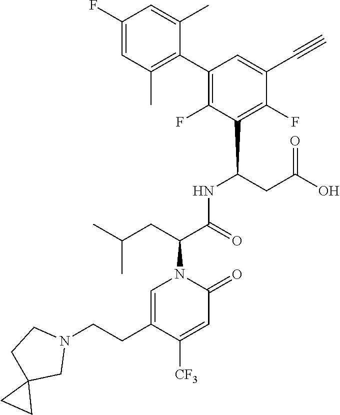 [SMILES: C#Cc1cc(-c2c(C)cc(F)cc2C)c(F)c([C@@H](CC(=O)O)NC(=O)[C@H](CC(C)C)n2cc(CCN3CCC4(CC4)C3)c(C(F)(F)F)cc2=O)c1F] | 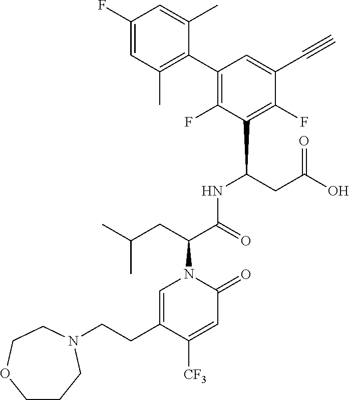 [SMILES: C#Cc1cc(-c2c(C)cc(F)cc2C)c(F)c([C@@H](CC(=O)O)NC(=O)[C@H](CC(C)C)n2cc(CCN3CCCOCC3)c(C(F)(F)F)cc2=O)c1F] |
| 157 | 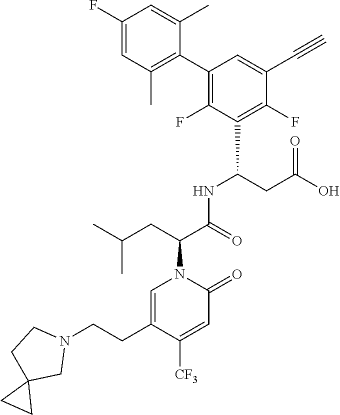 [SMILES: C#Cc1cc(-c2c(C)cc(F)cc2C)c(F)c([C@H](CC(=O)O)NC(=O)[C@H](CC(C)C)n2cc(CCN3CCC4(CC4)C3)c(C(F)(F)F)cc2=O)c1F] | 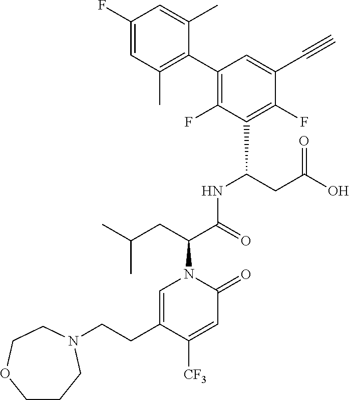 [SMILES: C#Cc1cc(-c2c(C)cc(F)cc2C)c(F)c([C@H](CC(=O)O)NC(=O)[C@H](CC(C)C)n2cc(CCN3CCCOCC3)c(C(F)(F)F)cc2=O)c1F] |
| 158 | 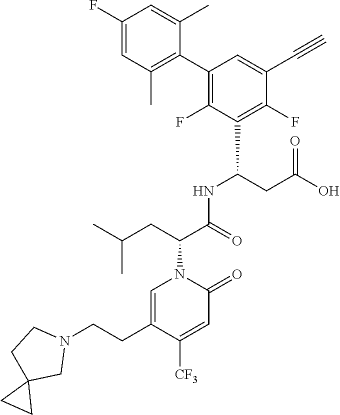 [SMILES: C#Cc1cc(-c2c(C)cc(F)cc2C)c(F)c([C@H](CC(=O)O)NC(=O)[C@@H](CC(C)C)n2cc(CCN3CCC4(CC4)C3)c(C(F)(F)F)cc2=O)c1F] | 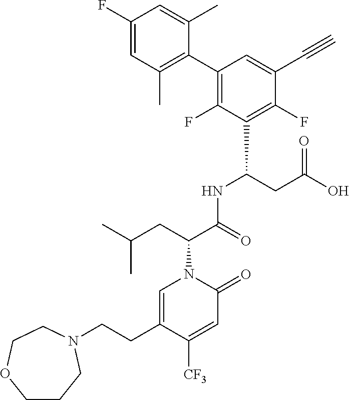 [SMILES: C#Cc1cc(-c2c(C)cc(F)cc2C)c(F)c([C@H](CC(=O)O)NC(=O)[C@@H](CC(C)C)n2cc(CCN3CCCOCC3)c(C(F)(F)F)cc2=O)c1F] |
| 159 | 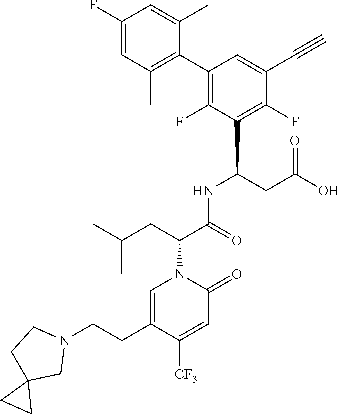 [SMILES: C#Cc1cc(-c2c(C)cc(F)cc2C)c(F)c([C@@H](CC(=O)O)NC(=O)[C@@H](CC(C)C)n2cc(CCN3CCC4(CC4)C3)c(C(F)(F)F)cc2=O)c1F] | 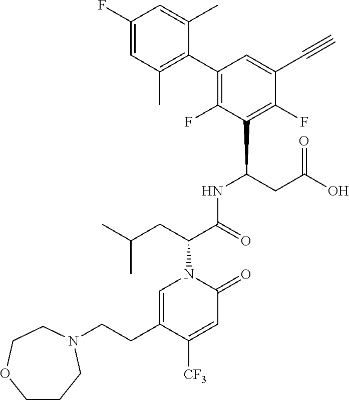 [SMILES: C#Cc1cc(-c2c(C)cc(F)cc2C)c(F)c([C@@H](CC(=O)O)NC(=O)[C@@H](CC(C)C)n2cc(CCN3CCCOCC3)c(C(F)(F)F)cc2=O)c1F] |
| 160 | 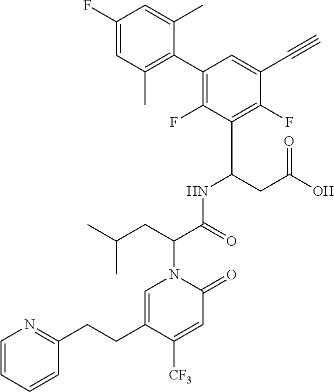 [SMILES: C#Cc1cc(-c2c(C)cc(F)cc2C)c(F)c(C(CC(=O)O)NC(=O)C(CC(C)C)n2cc(CCc3ccccn3)c(C(F)(F)F)cc2=O)c1F] | 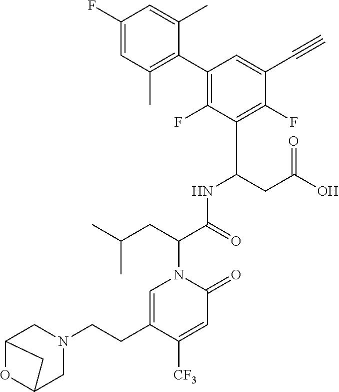 [SMILES: C#Cc1cc(-c2c(C)cc(F)cc2C)c(F)c(C(CC(=O)O)NC(=O)C(CC(C)C)n2cc(CCN3CC4CC(C3)O4)c(C(F)(F)F)cc2=O)c1F] |
| 161 | 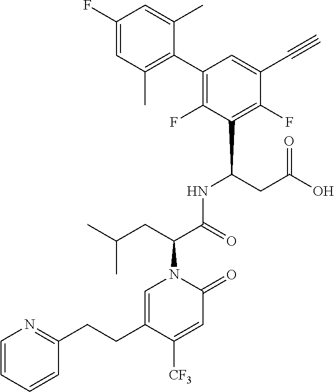 [SMILES: C#Cc1cc(-c2c(C)cc(F)cc2C)c(F)c([C@@H](CC(=O)O)NC(=O)[C@H](CC(C)C)n2cc(CCc3ccccn3)c(C(F)(F)F)cc2=O)c1F] | 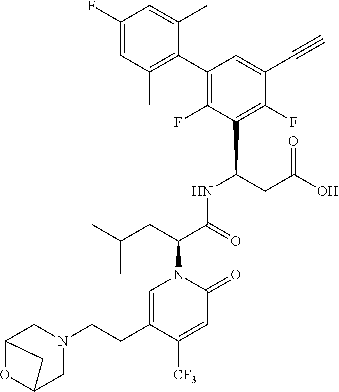 [SMILES: C#Cc1cc(-c2c(C)cc(F)cc2C)c(F)c([C@@H](CC(=O)O)NC(=O)[C@H](CC(C)C)n2cc(CCN3CC4CC(C3)O4)c(C(F)(F)F)cc2=O)c1F] |
| 162 | 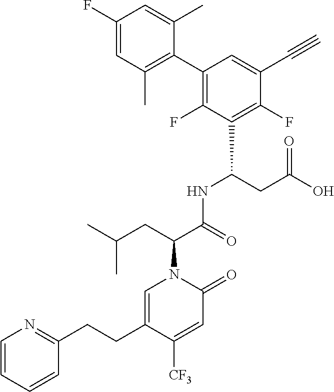 [SMILES: C#Cc1cc(-c2c(C)cc(F)cc2C)c(F)c([C@H](CC(=O)O)NC(=O)[C@H](CC(C)C)n2cc(CCc3ccccn3)c(C(F)(F)F)cc2=O)c1F] | 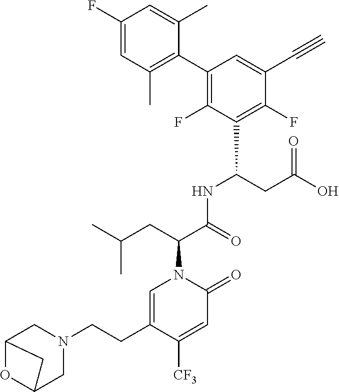 [SMILES: C#Cc1cc(-c2c(C)cc(F)cc2C)c(F)c([C@H](CC(=O)O)NC(=O)[C@H](CC(C)C)n2cc(CCN3CC4CC(C3)O4)c(C(F)(F)F)cc2=O)c1F] |
| 163 | 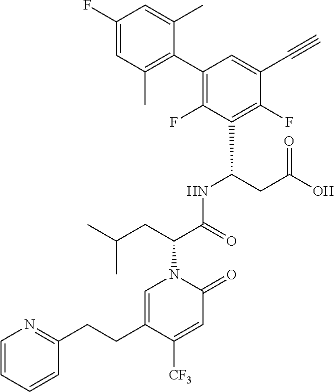 [SMILES: C#Cc1cc(-c2c(C)cc(F)cc2C)c(F)c([C@H](CC(=O)O)NC(=O)[C@@H](CC(C)C)n2cc(CCc3ccccn3)c(C(F)(F)F)cc2=O)c1F] | 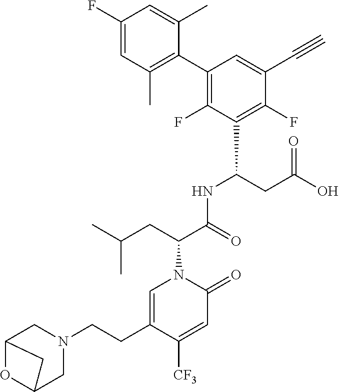 [SMILES: C#Cc1cc(-c2c(C)cc(F)cc2C)c(F)c([C@H](CC(=O)O)NC(=O)[C@@H](CC(C)C)n2cc(CCN3CC4CC(C3)O4)c(C(F)(F)F)cc2=O)c1F] |
| 164 | 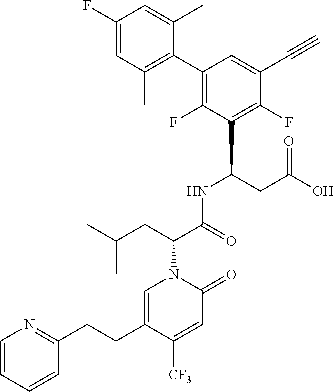 [SMILES: C#Cc1cc(-c2c(C)cc(F)cc2C)c(F)c([C@@H](CC(=O)O)NC(=O)[C@@H](CC(C)C)n2cc(CCc3ccccn3)c(C(F)(F)F)cc2=O)c1F] | 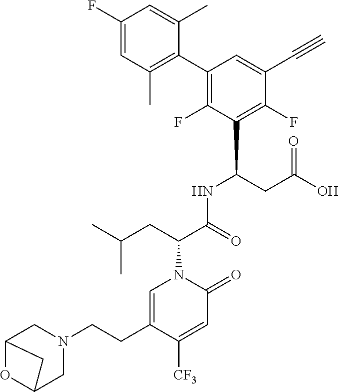 [SMILES: C#Cc1cc(-c2c(C)cc(F)cc2C)c(F)c([C@@H](CC(=O)O)NC(=O)[C@@H](CC(C)C)n2cc(CCN3CC4CC(C3)O4)c(C(F)(F)F)cc2=O)c1F] |
| 165 | 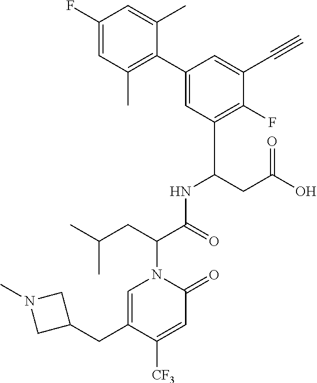 [SMILES: C#Cc1cc(-c2c(C)cc(F)cc2C)cc(C(CC(=O)O)NC(=O)C(CC(C)C)n2cc(CC3CN(C)C3)c(C(F)(F)F)cc2=O)c1F] | 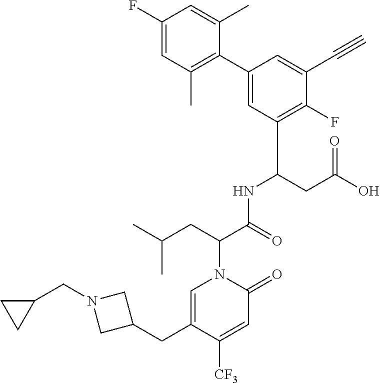 [SMILES: C#Cc1cc(-c2c(C)cc(F)cc2C)cc(C(CC(=O)O)NC(=O)C(CC(C)C)n2cc(CC3CN(CC4CC4)C3)c(C(F)(F)F)cc2=O)c1F] |
| 166 | 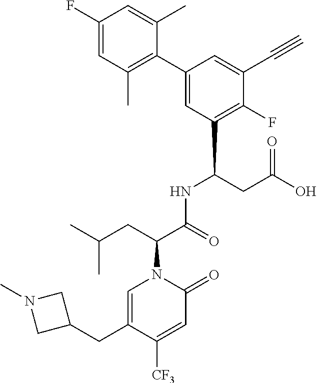 [SMILES: C#Cc1cc(-c2c(C)cc(F)cc2C)cc([C@@H](CC(=O)O)NC(=O)[C@H](CC(C)C)n2cc(CC3CN(C)C3)c(C(F)(F)F)cc2=O)c1F] | 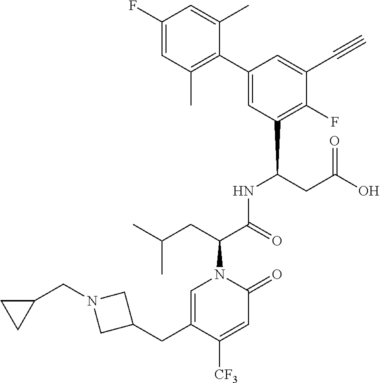 [SMILES: C#Cc1cc(-c2c(C)cc(F)cc2C)cc([C@@H](CC(=O)O)NC(=O)[C@H](CC(C)C)n2cc(CC3CN(CC4CC4)C3)c(C(F)(F)F)cc2=O)c1F] |
| 167 | 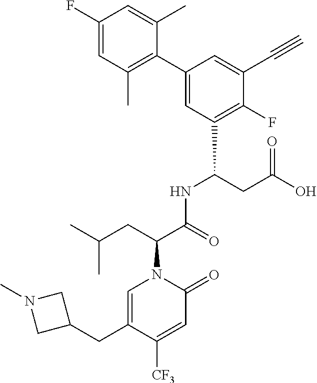 [SMILES: C#Cc1cc(-c2c(C)cc(F)cc2C)cc([C@H](CC(=O)O)NC(=O)[C@H](CC(C)C)n2cc(CC3CN(C)C3)c(C(F)(F)F)cc2=O)c1F] | 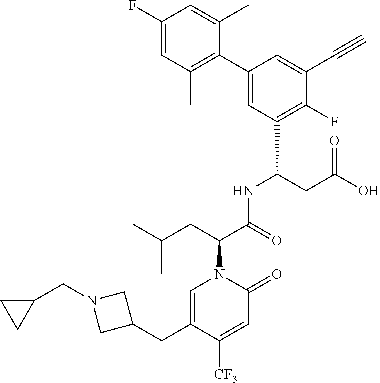 [SMILES: C#Cc1cc(-c2c(C)cc(F)cc2C)cc([C@H](CC(=O)O)NC(=O)[C@H](CC(C)C)n2cc(CC3CN(CC4CC4)C3)c(C(F)(F)F)cc2=O)c1F] |
| 168 | 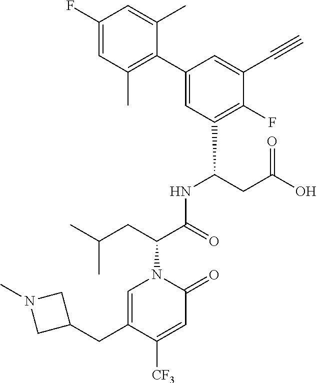 [SMILES: C#Cc1cc(-c2c(C)cc(F)cc2C)cc([C@H](CC(=O)O)NC(=O)[C@@H](CC(C)C)n2cc(CC3CN(C)C3)c(C(F)(F)F)cc2=O)c1F] | 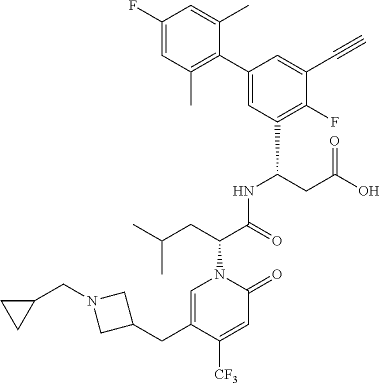 [SMILES: C#Cc1cc(-c2c(C)cc(F)cc2C)cc([C@H](CC(=O)O)NC(=O)[C@@H](CC(C)C)n2cc(CC3CN(CC4CC4)C3)c(C(F)(F)F)cc2=O)c1F] |
| 169 | 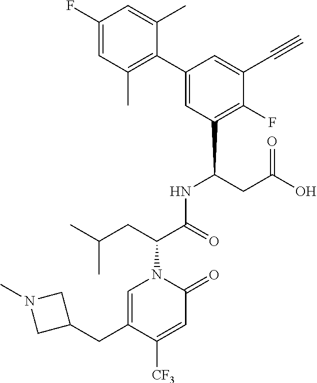 [SMILES: C#Cc1cc(-c2c(C)cc(F)cc2C)cc([C@@H](CC(=O)O)NC(=O)[C@@H](CC(C)C)n2cc(CC3CN(C)C3)c(C(F)(F)F)cc2=O)c1F] | 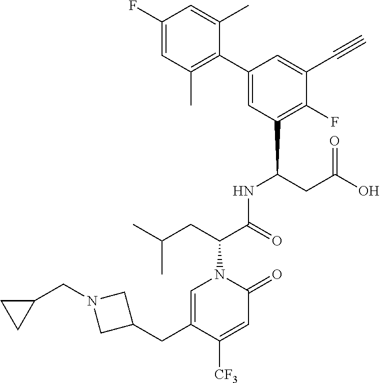 [SMILES: C#Cc1cc(-c2c(C)cc(F)cc2C)cc([C@@H](CC(=O)O)NC(=O)[C@@H](CC(C)C)n2cc(CC3CN(CC4CC4)C3)c(C(F)(F)F)cc2=O)c1F] |
| 170 | 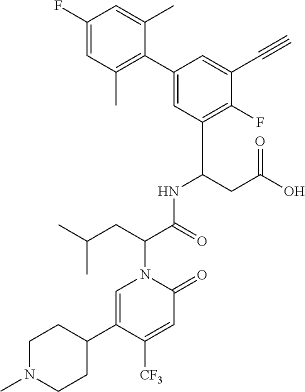 [SMILES: C#Cc1cc(-c2c(C)cc(F)cc2C)cc(C(CC(=O)O)NC(=O)C(CC(C)C)n2cc(C3CCN(C)CC3)c(C(F)(F)F)cc2=O)c1F] | 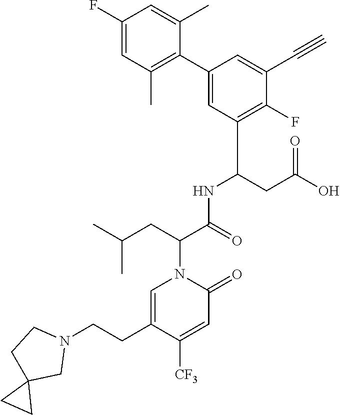 [SMILES: C#Cc1cc(-c2c(C)cc(F)cc2C)cc(C(CC(=O)O)NC(=O)C(CC(C)C)n2cc(CCN3CCC4(CC4)C3)c(C(F)(F)F)cc2=O)c1F] |
| 171 | 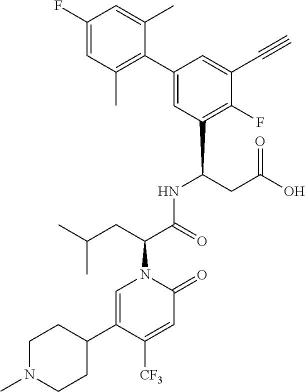 [SMILES: C#Cc1cc(-c2c(C)cc(F)cc2C)cc([C@@H](CC(=O)O)NC(=O)[C@H](CC(C)C)n2cc(C3CCN(C)CC3)c(C(F)(F)F)cc2=O)c1F] | 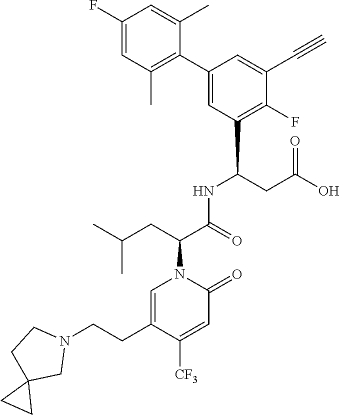 [SMILES: C#Cc1cc(-c2c(C)cc(F)cc2C)cc([C@@H](CC(=O)O)NC(=O)[C@H](CC(C)C)n2cc(CCN3CCC4(CC4)C3)c(C(F)(F)F)cc2=O)c1F] |
| 172 | 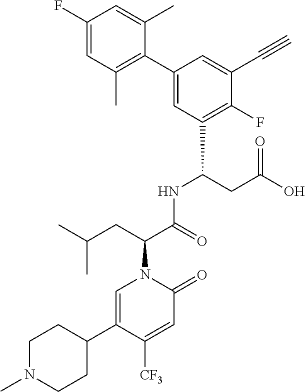 [SMILES: C#Cc1cc(-c2c(C)cc(F)cc2C)cc([C@H](CC(=O)O)NC(=O)[C@H](CC(C)C)n2cc(C3CCN(C)CC3)c(C(F)(F)F)cc2=O)c1F] | 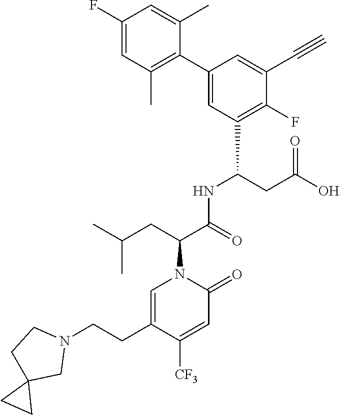 [SMILES: C#Cc1cc(-c2c(C)cc(F)cc2C)cc([C@H](CC(=O)O)NC(=O)[C@H](CC(C)C)n2cc(CCN3CCC4(CC4)C3)c(C(F)(F)F)cc2=O)c1F] |
| 173 | 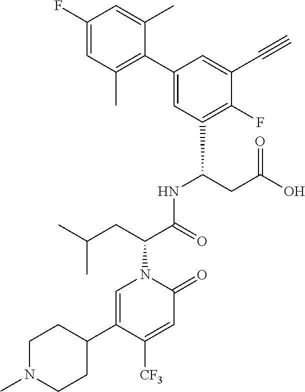 [SMILES: C#Cc1cc(-c2c(C)cc(F)cc2C)cc([C@H](CC(=O)O)NC(=O)[C@@H](CC(C)C)n2cc(C3CCN(C)CC3)c(C(F)(F)F)cc2=O)c1F] | 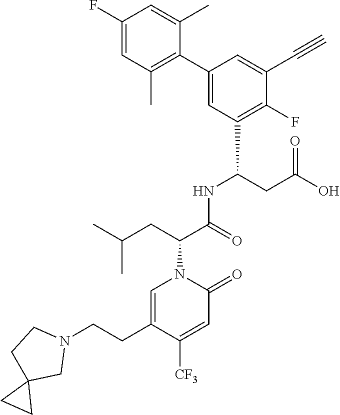 [SMILES: C#Cc1cc(-c2c(C)cc(F)cc2C)cc([C@H](CC(=O)O)NC(=O)[C@@H](CC(C)C)n2cc(CCN3CCC4(CC4)C3)c(C(F)(F)F)cc2=O)c1F] |
| 174 | 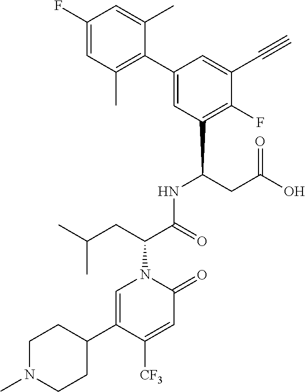 [SMILES: C#Cc1cc(-c2c(C)cc(F)cc2C)cc([C@@H](CC(=O)O)NC(=O)[C@@H](CC(C)C)n2cc(C3CCN(C)CC3)c(C(F)(F)F)cc2=O)c1F] | 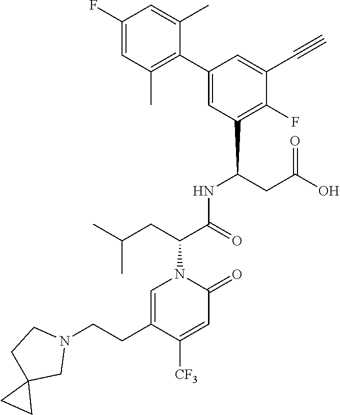 [SMILES: C#Cc1cc(-c2c(C)cc(F)cc2C)cc([C@@H](CC(=O)O)NC(=O)[C@@H](CC(C)C)n2cc(CCN3CCC4(CC4)C3)c(C(F)(F)F)cc2=O)c1F] |
| 175 | 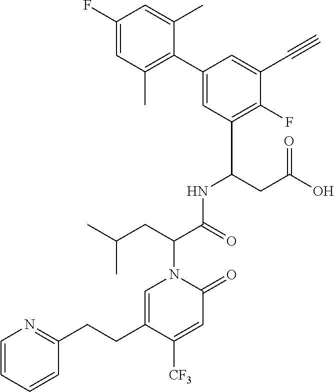 [SMILES: C#Cc1cc(-c2c(C)cc(F)cc2C)cc(C(CC(=O)O)NC(=O)C(CC(C)C)n2cc(CCc3ccccn3)c(C(F)(F)F)cc2=O)c1F] | 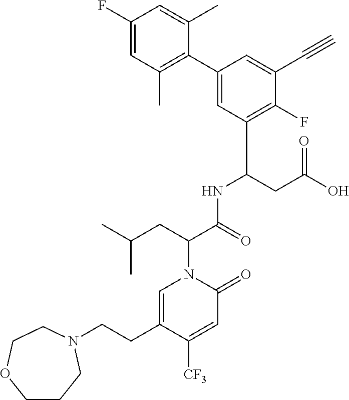 [SMILES: C#Cc1cc(-c2c(C)cc(F)cc2C)cc(C(CC(=O)O)NC(=O)C(CC(C)C)n2cc(CCN3CCCOCC3)c(C(F)(F)F)cc2=O)c1F] |
| 176 | 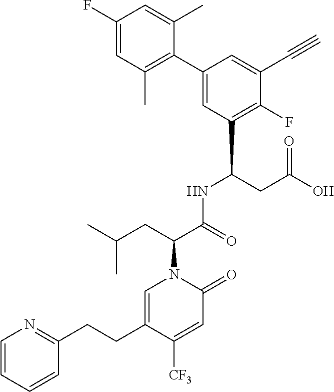 [SMILES: C#Cc1cc(-c2c(C)cc(F)cc2C)cc([C@@H](CC(=O)O)NC(=O)[C@H](CC(C)C)n2cc(CCc3ccccn3)c(C(F)(F)F)cc2=O)c1F] | 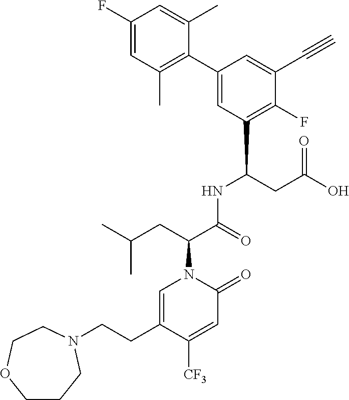 [SMILES: C#Cc1cc(-c2c(C)cc(F)cc2C)cc([C@@H](CC(=O)O)NC(=O)[C@H](CC(C)C)n2cc(CCN3CCCOCC3)c(C(F)(F)F)cc2=O)c1F] |
| 177 | 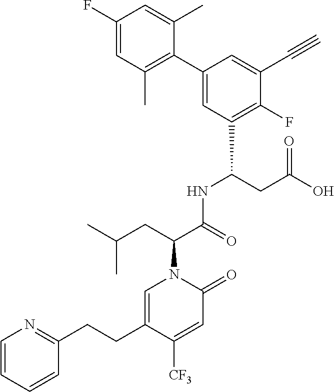 [SMILES: C#Cc1cc(-c2c(C)cc(F)cc2C)cc([C@H](CC(=O)O)NC(=O)[C@H](CC(C)C)n2cc(CCc3ccccn3)c(C(F)(F)F)cc2=O)c1F] | 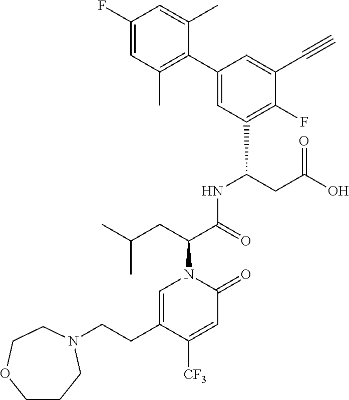 [SMILES: C#Cc1cc(-c2c(C)cc(F)cc2C)cc([C@H](CC(=O)O)NC(=O)[C@H](CC(C)C)n2cc(CCN3CCCOCC3)c(C(F)(F)F)cc2=O)c1F] |
| 178 | 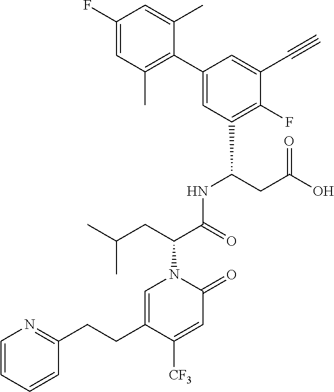 [SMILES: C#Cc1cc(-c2c(C)cc(F)cc2C)cc([C@H](CC(=O)O)NC(=O)[C@@H](CC(C)C)n2cc(CCc3ccccn3)c(C(F)(F)F)cc2=O)c1F] | 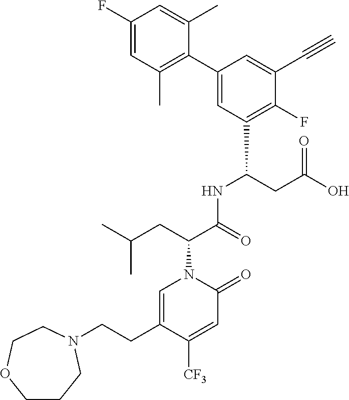 [SMILES: C#Cc1cc(-c2c(C)cc(F)cc2C)cc([C@H](CC(=O)O)NC(=O)[C@@H](CC(C)C)n2cc(CCN3CCCOCC3)c(C(F)(F)F)cc2=O)c1F] |
| 179 | 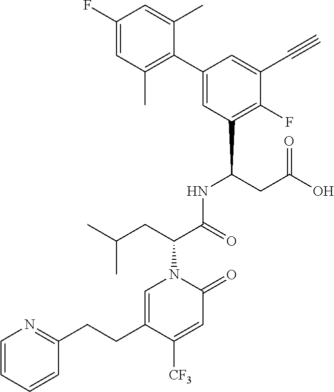 [SMILES: C#Cc1cc(-c2c(C)cc(F)cc2C)cc([C@@H](CC(=O)O)NC(=O)[C@@H](CC(C)C)n2cc(CCc3ccccn3)c(C(F)(F)F)cc2=O)c1F] | 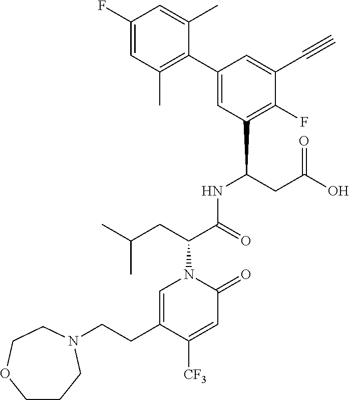 [SMILES: C#Cc1cc(-c2c(C)cc(F)cc2C)cc([C@@H](CC(=O)O)NC(=O)[C@@H](CC(C)C)n2cc(CCN3CCCOCC3)c(C(F)(F)F)cc2=O)c1F] |
| 180 | 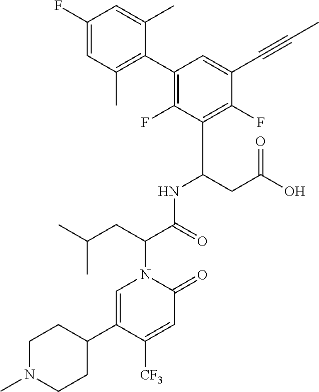 [SMILES: CC#Cc1cc(-c2c(C)cc(F)cc2C)c(F)c(C(CC(=O)O)NC(=O)C(CC(C)C)n2cc(C3CCN(C)CC3)c(C(F)(F)F)cc2=O)c1F] | 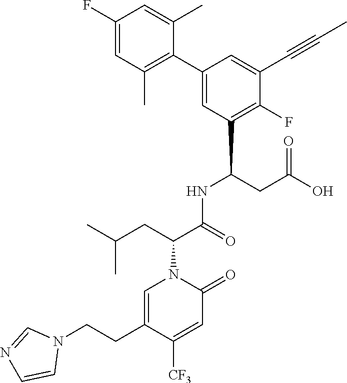 [SMILES: CC#Cc1cc(-c2c(C)cc(F)cc2C)cc([C@@H](CC(=O)O)NC(=O)[C@@H](CC(C)C)n2cc(CCn3ccnc3)c(C(F)(F)F)cc2=O)c1F] |
| 181 | 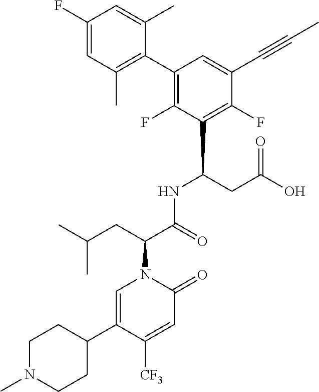 [SMILES: CC#Cc1cc(-c2c(C)cc(F)cc2C)c(F)c([C@@H](CC(=O)O)NC(=O)[C@H](CC(C)C)n2cc(C3CCN(C)CC3)c(C(F)(F)F)cc2=O)c1F] | 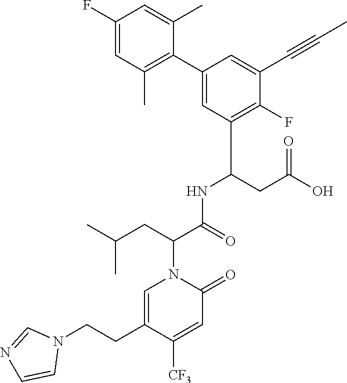 [SMILES: CC#Cc1cc(-c2c(C)cc(F)cc2C)cc(C(CC(=O)O)NC(=O)C(CC(C)C)n2cc(CCn3ccnc3)c(C(F)(F)F)cc2=O)c1F] |
| 182 | 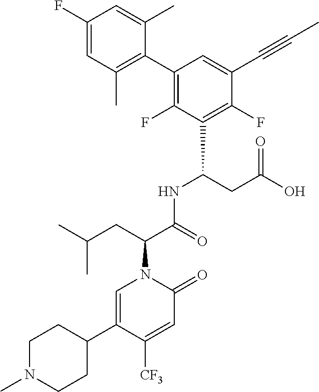 [SMILES: CC#Cc1cc(-c2c(C)cc(F)cc2C)c(F)c([C@H](CC(=O)O)NC(=O)[C@H](CC(C)C)n2cc(C3CCN(C)CC3)c(C(F)(F)F)cc2=O)c1F] | 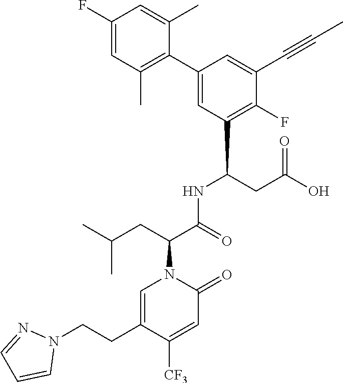 [SMILES: CC#Cc1cc(-c2c(C)cc(F)cc2C)cc([C@@H](CC(=O)O)NC(=O)[C@H](CC(C)C)n2cc(CCn3cccn3)c(C(F)(F)F)cc2=O)c1F] |
| 183 | 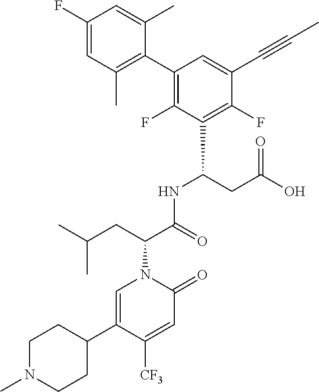 [SMILES: CC#Cc1cc(-c2c(C)cc(F)cc2C)c(F)c([C@H](CC(=O)O)NC(=O)[C@@H](CC(C)C)n2cc(C3CCN(C)CC3)c(C(F)(F)F)cc2=O)c1F] | 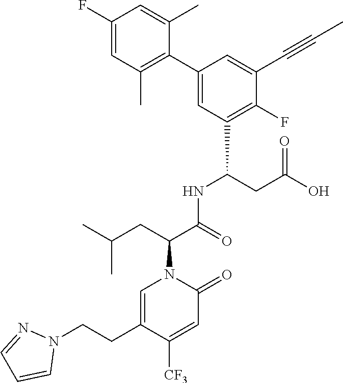 [SMILES: CC#Cc1cc(-c2c(C)cc(F)cc2C)cc([C@H](CC(=O)O)NC(=O)[C@H](CC(C)C)n2cc(CCn3cccn3)c(C(F)(F)F)cc2=O)c1F] |
| 184 | 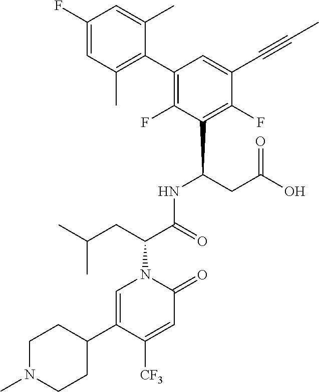 [SMILES: CC#Cc1cc(-c2c(C)cc(F)cc2C)c(F)c([C@@H](CC(=O)O)NC(=O)[C@@H](CC(C)C)n2cc(C3CCN(C)CC3)c(C(F)(F)F)cc2=O)c1F] | 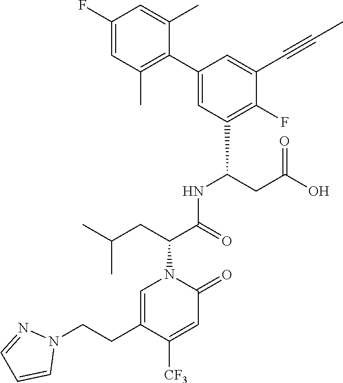 [SMILES: CC#Cc1cc(-c2c(C)cc(F)cc2C)cc([C@H](CC(=O)O)NC(=O)[C@@H](CC(C)C)n2cc(CCn3cccn3)c(C(F)(F)F)cc2=O)c1F] |
| 185 | 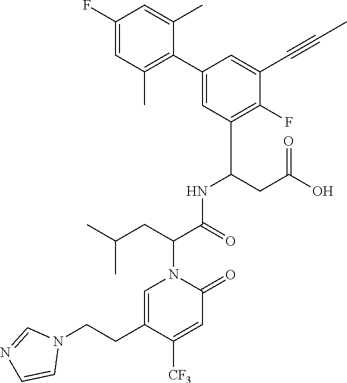 [SMILES: CC#Cc1cc(-c2c(C)cc(F)cc2C)cc(C(CC(=O)O)NC(=O)C(CC(C)C)n2cc(CCn3ccnc3)c(C(F)(F)F)cc2=O)c1F] | 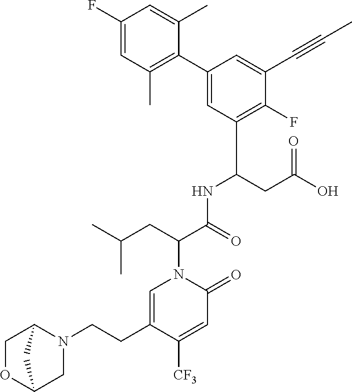 [SMILES: CC#Cc1cc(-c2c(C)cc(F)cc2C)cc(C(CC(=O)O)NC(=O)C(CC(C)C)n2cc(CCN3C[C@@H]4C[C@H]3CO4)c(C(F)(F)F)cc2=O)c1F] |
| 186 | 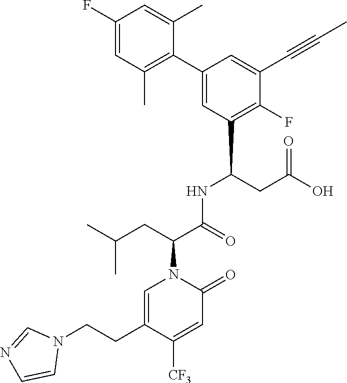 [SMILES: CC#Cc1cc(-c2c(C)cc(F)cc2C)cc([C@@H](CC(=O)O)NC(=O)[C@H](CC(C)C)n2cc(CCn3ccnc3)c(C(F)(F)F)cc2=O)c1F] | 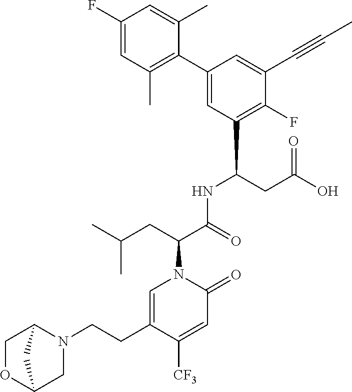 [SMILES: CC#Cc1cc(-c2c(C)cc(F)cc2C)cc([C@@H](CC(=O)O)NC(=O)[C@H](CC(C)C)n2cc(CCN3C[C@@H]4C[C@H]3CO4)c(C(F)(F)F)cc2=O)c1F] |
| 187 | 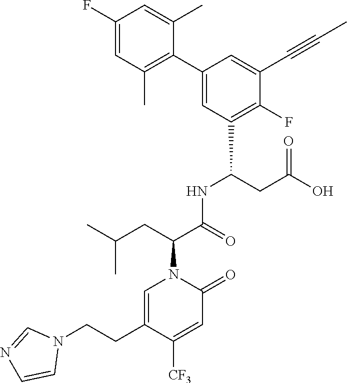 [SMILES: CC#Cc1cc(-c2c(C)cc(F)cc2C)cc([C@H](CC(=O)O)NC(=O)[C@H](CC(C)C)n2cc(CCn3ccnc3)c(C(F)(F)F)cc2=O)c1F] | 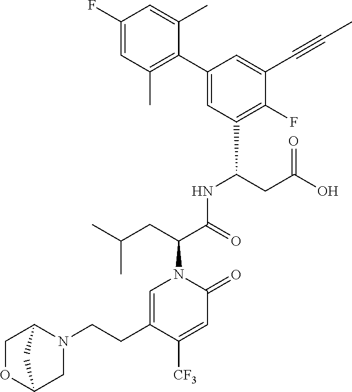 [SMILES: CC#Cc1cc(-c2c(C)cc(F)cc2C)cc([C@H](CC(=O)O)NC(=O)[C@H](CC(C)C)n2cc(CCN3C[C@@H]4C[C@H]3CO4)c(C(F)(F)F)cc2=O)c1F] |
| 188 | 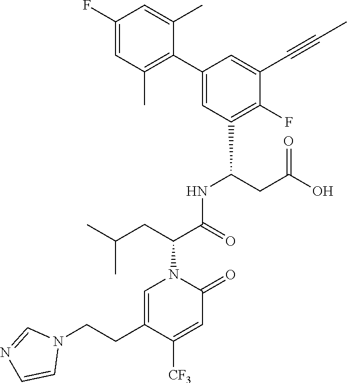 [SMILES: CC#Cc1cc(-c2c(C)cc(F)cc2C)cc([C@H](CC(=O)O)NC(=O)[C@@H](CC(C)C)n2cc(CCn3ccnc3)c(C(F)(F)F)cc2=O)c1F] | 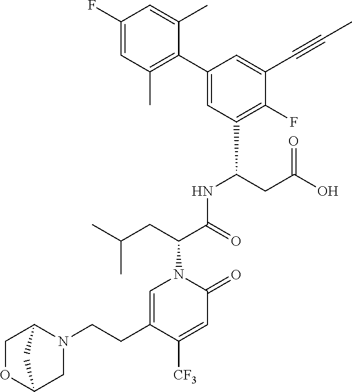 [SMILES: CC#Cc1cc(-c2c(C)cc(F)cc2C)cc([C@H](CC(=O)O)NC(=O)[C@@H](CC(C)C)n2cc(CCN3C[C@@H]4C[C@H]3CO4)c(C(F)(F)F)cc2=O)c1F] |
| 189 | 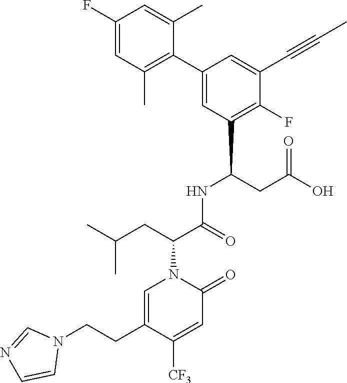 [SMILES: CC#Cc1cc(-c2c(C)cc(F)cc2C)cc([C@@H](CC(=O)O)NC(=O)[C@@H](CC(C)C)n2cc(CCn3ccnc3)c(C(F)(F)F)cc2=O)c1F] | 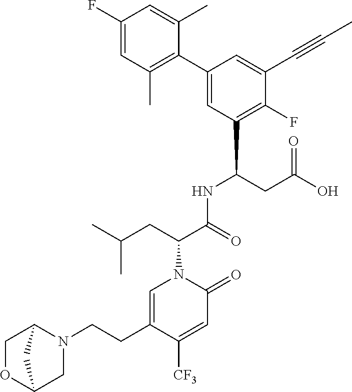 [SMILES: CC#Cc1cc(-c2c(C)cc(F)cc2C)cc([C@@H](CC(=O)O)NC(=O)[C@@H](CC(C)C)n2cc(CCN3C[C@@H]4C[C@H]3CO4)c(C(F)(F)F)cc2=O)c1F] |
| 190 | 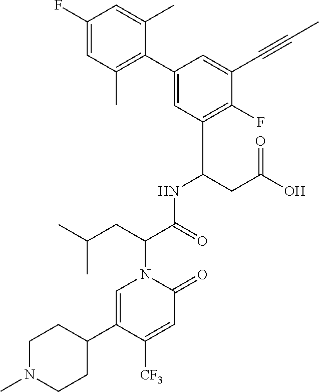 [SMILES: CC#Cc1cc(-c2c(C)cc(F)cc2C)cc(C(CC(=O)O)NC(=O)C(CC(C)C)n2cc(C3CCN(C)CC3)c(C(F)(F)F)cc2=O)c1F] | 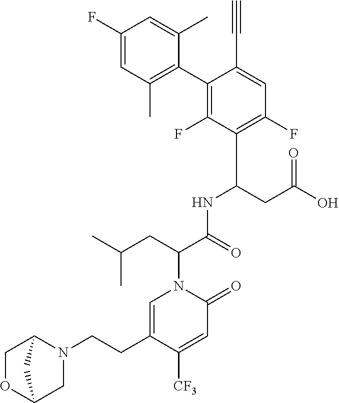 [SMILES: C#Cc1cc(F)c(C(CC(=O)O)NC(=O)C(CC(C)C)n2cc(CCN3C[C@@H]4C[C@H]3CO4)c(C(F)(F)F)cc2=O)c(F)c1-c1c(C)cc(F)cc1C] |
| 191 | 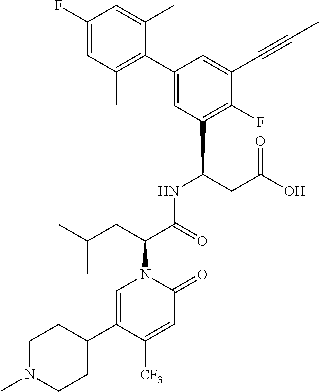 [SMILES: CC#Cc1cc(-c2c(C)cc(F)cc2C)cc([C@@H](CC(=O)O)NC(=O)[C@H](CC(C)C)n2cc(C3CCN(C)CC3)c(C(F)(F)F)cc2=O)c1F] | 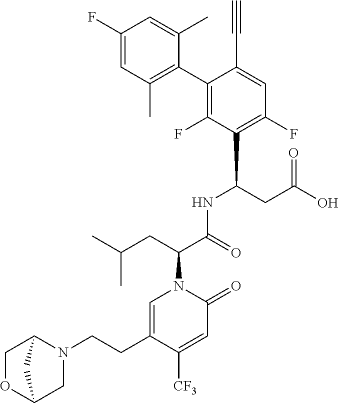 [SMILES: C#Cc1cc(F)c([C@@H](CC(=O)O)NC(=O)[C@H](CC(C)C)n2cc(CCN3C[C@@H]4C[C@H]3CO4)c(C(F)(F)F)cc2=O)c(F)c1-c1c(C)cc(F)cc1C] |
| 192 | 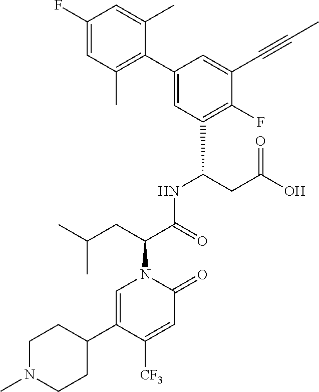 [SMILES: CC#Cc1cc(-c2c(C)cc(F)cc2C)cc([C@H](CC(=O)O)NC(=O)[C@H](CC(C)C)n2cc(C3CCN(C)CC3)c(C(F)(F)F)cc2=O)c1F] | 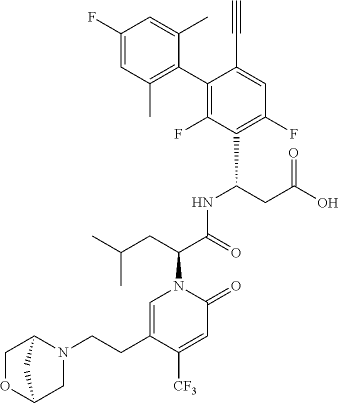 [SMILES: C#Cc1cc(F)c([C@H](CC(=O)O)NC(=O)[C@H](CC(C)C)n2cc(CCN3C[C@@H]4C[C@H]3CO4)c(C(F)(F)F)cc2=O)c(F)c1-c1c(C)cc(F)cc1C] |
| 193 | 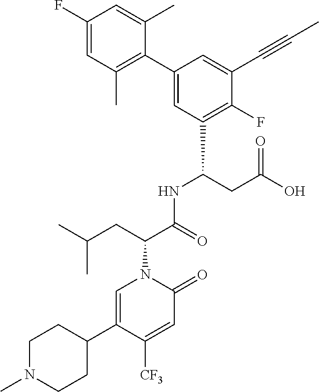 [SMILES: CC#Cc1cc(-c2c(C)cc(F)cc2C)cc([C@H](CC(=O)O)NC(=O)[C@@H](CC(C)C)n2cc(C3CCN(C)CC3)c(C(F)(F)F)cc2=O)c1F] | 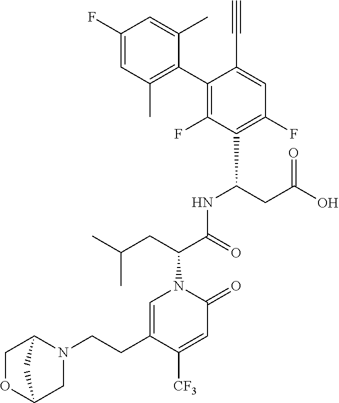 [SMILES: C#Cc1cc(F)c([C@H](CC(=O)O)NC(=O)[C@@H](CC(C)C)n2cc(CCN3C[C@@H]4C[C@H]3CO4)c(C(F)(F)F)cc2=O)c(F)c1-c1c(C)cc(F)cc1C] |
| 194 | 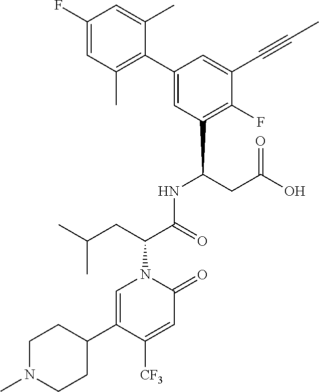 [SMILES: CC#Cc1cc(-c2c(C)cc(F)cc2C)cc([C@@H](CC(=O)O)NC(=O)[C@@H](CC(C)C)n2cc(C3CCN(C)CC3)c(C(F)(F)F)cc2=O)c1F] | 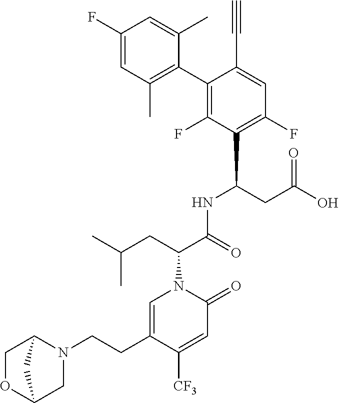 [SMILES: C#Cc1cc(F)c([C@@H](CC(=O)O)NC(=O)[C@@H](CC(C)C)n2cc(CCN3C[C@@H]4C[C@H]3CO4)c(C(F)(F)F)cc2=O)c(F)c1-c1c(C)cc(F)cc1C] |
| 195 | 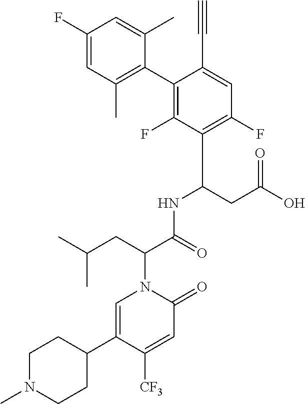 [SMILES: C#Cc1cc(F)c(C(CC(=O)O)NC(=O)C(CC(C)C)n2cc(C3CCN(C)CC3)c(C(F)(F)F)cc2=O)c(F)c1-c1c(C)cc(F)cc1C] | 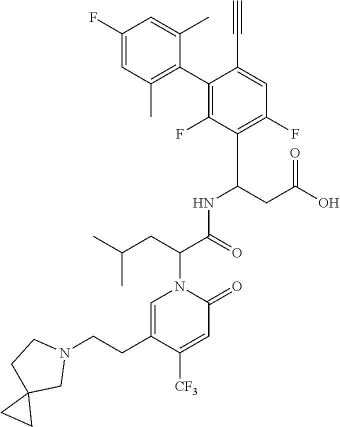 [SMILES: C#Cc1cc(F)c(C(CC(=O)O)NC(=O)C(CC(C)C)n2cc(CCN3CCC4(CC4)C3)c(C(F)(F)F)cc2=O)c(F)c1-c1c(C)cc(F)cc1C] |
| 196 | 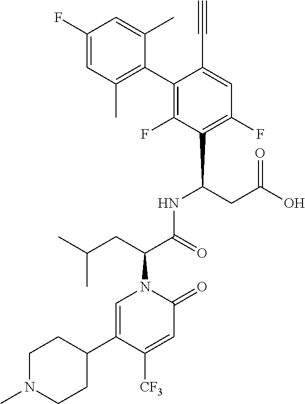 [SMILES: C#Cc1cc(F)c([C@@H](CC(=O)O)NC(=O)[C@H](CC(C)C)n2cc(C3CCN(C)CC3)c(C(F)(F)F)cc2=O)c(F)c1-c1c(C)cc(F)cc1C] | 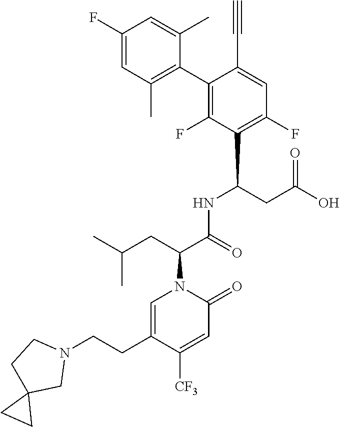 [SMILES: C#Cc1cc(F)c([C@@H](CC(=O)O)NC(=O)[C@H](CC(C)C)n2cc(CCN3CCC4(CC4)C3)c(C(F)(F)F)cc2=O)c(F)c1-c1c(C)cc(F)cc1C] |
| 197 | 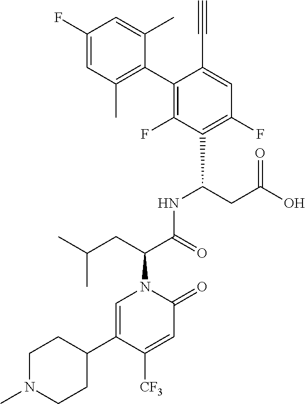 [SMILES: C#Cc1cc(F)c([C@H](CC(=O)O)NC(=O)[C@H](CC(C)C)n2cc(C3CCN(C)CC3)c(C(F)(F)F)cc2=O)c(F)c1-c1c(C)cc(F)cc1C] | 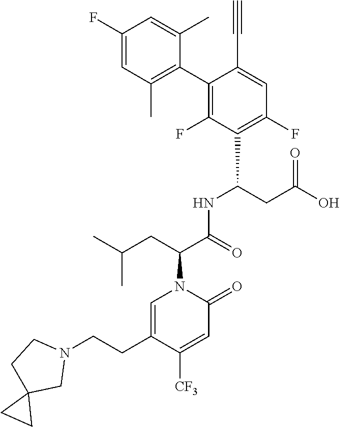 [SMILES: C#Cc1cc(F)c([C@H](CC(=O)O)NC(=O)[C@H](CC(C)C)n2cc(CCN3CCC4(CC4)C3)c(C(F)(F)F)cc2=O)c(F)c1-c1c(C)cc(F)cc1C] |
| 198 | 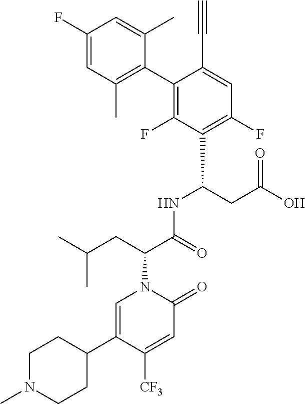 [SMILES: C#Cc1cc(F)c([C@H](CC(=O)O)NC(=O)[C@@H](CC(C)C)n2cc(C3CCN(C)CC3)c(C(F)(F)F)cc2=O)c(F)c1-c1c(C)cc(F)cc1C] | 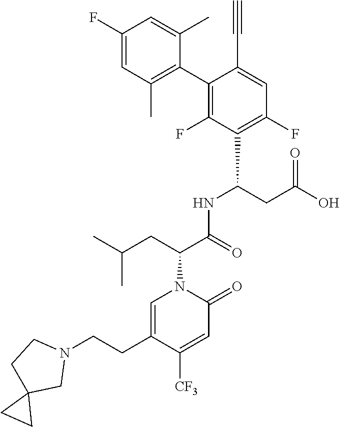 [SMILES: C#Cc1cc(F)c([C@H](CC(=O)O)NC(=O)[C@@H](CC(C)C)n2cc(CCN3CCC4(CC4)C3)c(C(F)(F)F)cc2=O)c(F)c1-c1c(C)cc(F)cc1C] |
| 199 | 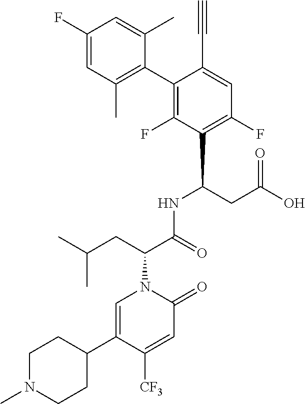 [SMILES: C#Cc1cc(F)c([C@@H](CC(=O)O)NC(=O)[C@@H](CC(C)C)n2cc(C3CCN(C)CC3)c(C(F)(F)F)cc2=O)c(F)c1-c1c(C)cc(F)cc1C] | 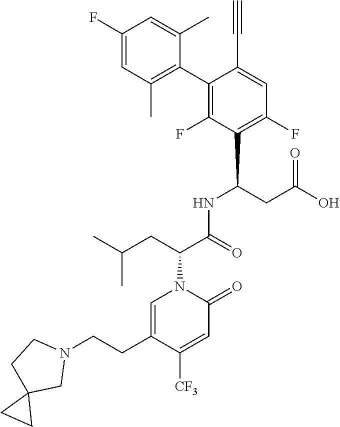 [SMILES: C#Cc1cc(F)c([C@@H](CC(=O)O)NC(=O)[C@@H](CC(C)C)n2cc(CCN3CCC4(CC4)C3)c(C(F)(F)F)cc2=O)c(F)c1-c1c(C)cc(F)cc1C] |
| 200 | 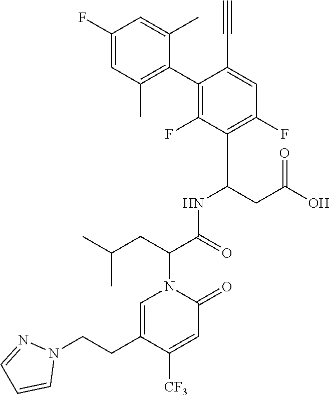 [SMILES: C#Cc1cc(F)c(C(CC(=O)O)NC(=O)C(CC(C)C)n2cc(CCn3cccn3)c(C(F)(F)F)cc2=O)c(F)c1-c1c(C)cc(F)cc1C] | 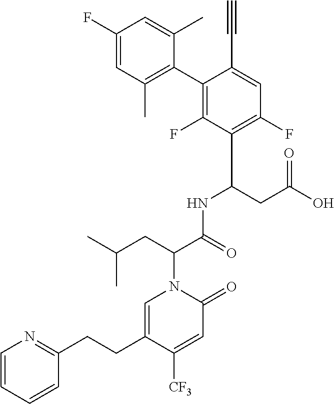 [SMILES: C#Cc1cc(F)c(C(CC(=O)O)NC(=O)C(CC(C)C)n2cc(CCc3ccccn3)c(C(F)(F)F)cc2=O)c(F)c1-c1c(C)cc(F)cc1C] |
| 201 | 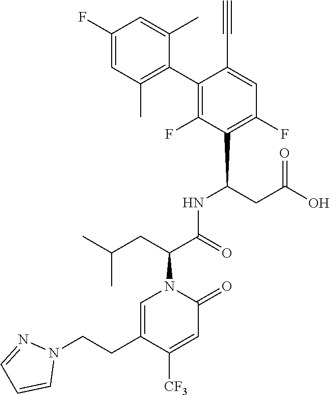 [SMILES: C#Cc1cc(F)c([C@@H](CC(=O)O)NC(=O)[C@H](CC(C)C)n2cc(CCn3cccn3)c(C(F)(F)F)cc2=O)c(F)c1-c1c(C)cc(F)cc1C] | 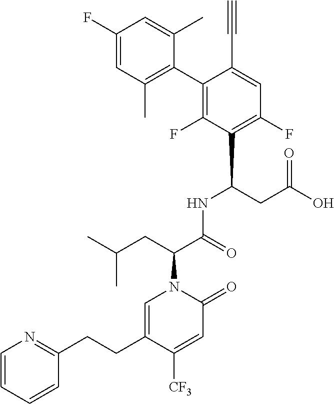 [SMILES: C#Cc1cc(F)c([C@@H](CC(=O)O)NC(=O)[C@H](CC(C)C)n2cc(CCc3ccccn3)c(C(F)(F)F)cc2=O)c(F)c1-c1c(C)cc(F)cc1C] |
| 202 | 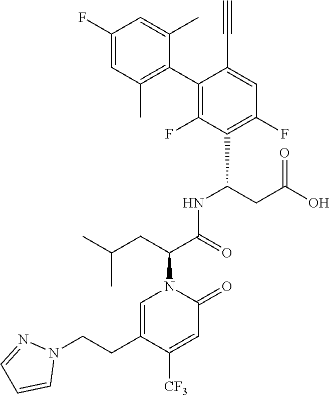 [SMILES: C#Cc1cc(F)c([C@H](CC(=O)O)NC(=O)[C@H](CC(C)C)n2cc(CCn3cccn3)c(C(F)(F)F)cc2=O)c(F)c1-c1c(C)cc(F)cc1C] | 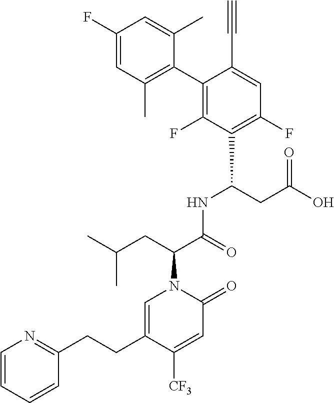 [SMILES: C#Cc1cc(F)c([C@H](CC(=O)O)NC(=O)[C@H](CC(C)C)n2cc(CCc3ccccn3)c(C(F)(F)F)cc2=O)c(F)c1-c1c(C)cc(F)cc1C] |
| 203 | 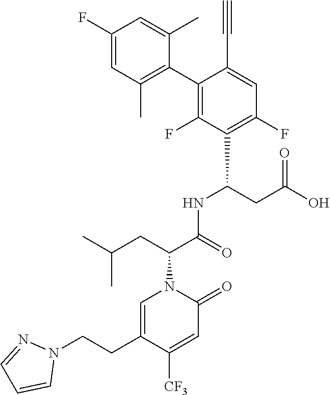 [SMILES: C#Cc1cc(F)c([C@H](CC(=O)O)NC(=O)[C@@H](CC(C)C)n2cc(CCn3cccn3)c(C(F)(F)F)cc2=O)c(F)c1-c1c(C)cc(F)cc1C] | 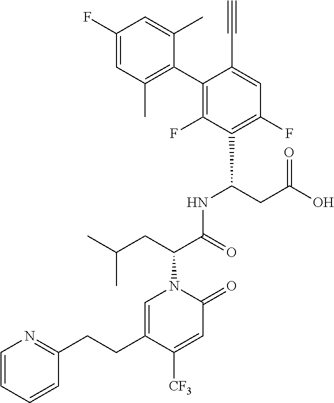 [SMILES: C#Cc1cc(F)c([C@H](CC(=O)O)NC(=O)[C@@H](CC(C)C)n2cc(CCc3ccccn3)c(C(F)(F)F)cc2=O)c(F)c1-c1c(C)cc(F)cc1C] |
| 204 | 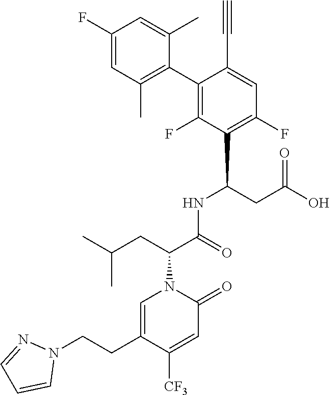 [SMILES: C#Cc1cc(F)c([C@@H](CC(=O)O)NC(=O)[C@@H](CC(C)C)n2cc(CCn3cccn3)c(C(F)(F)F)cc2=O)c(F)c1-c1c(C)cc(F)cc1C] | 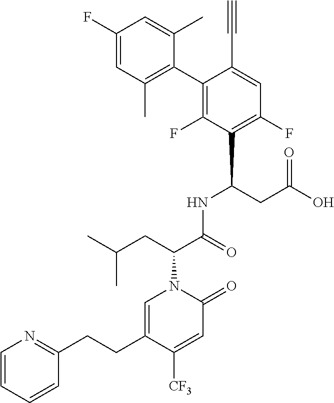 [SMILES: C#Cc1cc(F)c([C@@H](CC(=O)O)NC(=O)[C@@H](CC(C)C)n2cc(CCc3ccccn3)c(C(F)(F)F)cc2=O)c(F)c1-c1c(C)cc(F)cc1C] |
| 205 | 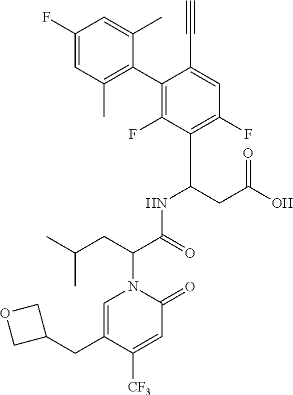 [SMILES: C#Cc1cc(F)c(C(CC(=O)O)NC(=O)C(CC(C)C)n2cc(CC3COC3)c(C(F)(F)F)cc2=O)c(F)c1-c1c(C)cc(F)cc1C] | 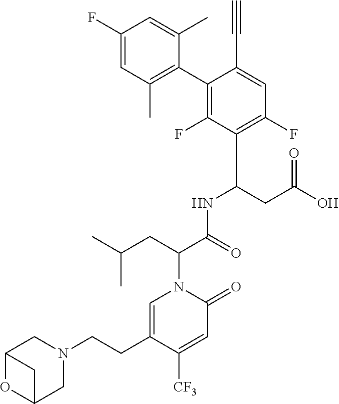 [SMILES: C#Cc1cc(F)c(C(CC(=O)O)NC(=O)C(CC(C)C)n2cc(CCN3CC4CC(C3)O4)c(C(F)(F)F)cc2=O)c(F)c1-c1c(C)cc(F)cc1C] |
| 206 | 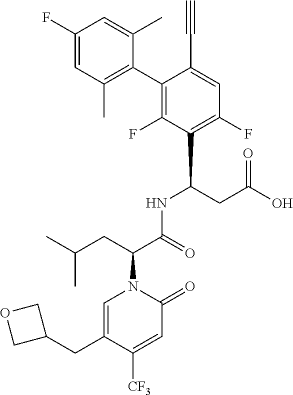 [SMILES: C#Cc1cc(F)c([C@@H](CC(=O)O)NC(=O)[C@H](CC(C)C)n2cc(CC3COC3)c(C(F)(F)F)cc2=O)c(F)c1-c1c(C)cc(F)cc1C] | 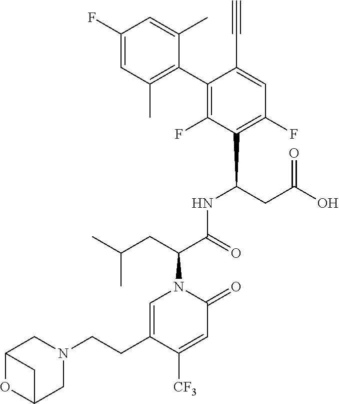 [SMILES: C#Cc1cc(F)c([C@@H](CC(=O)O)NC(=O)[C@H](CC(C)C)n2cc(CCN3CC4CC(C3)O4)c(C(F)(F)F)cc2=O)c(F)c1-c1c(C)cc(F)cc1C] |
| 207 | 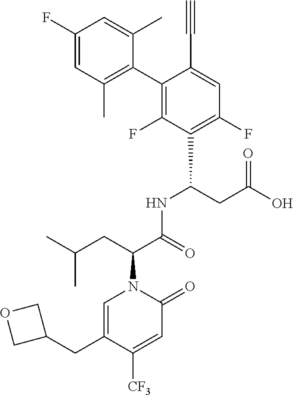 [SMILES: C#Cc1cc(F)c([C@H](CC(=O)O)NC(=O)[C@H](CC(C)C)n2cc(CC3COC3)c(C(F)(F)F)cc2=O)c(F)c1-c1c(C)cc(F)cc1C] | 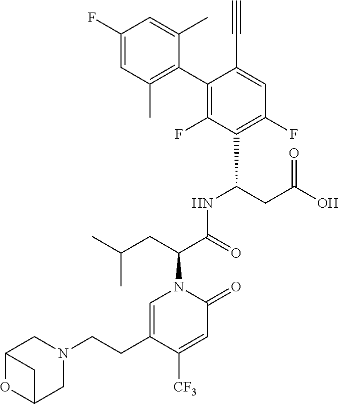 [SMILES: C#Cc1cc(F)c([C@H](CC(=O)O)NC(=O)[C@H](CC(C)C)n2cc(CCN3CC4CC(C3)O4)c(C(F)(F)F)cc2=O)c(F)c1-c1c(C)cc(F)cc1C] |
| 208 | 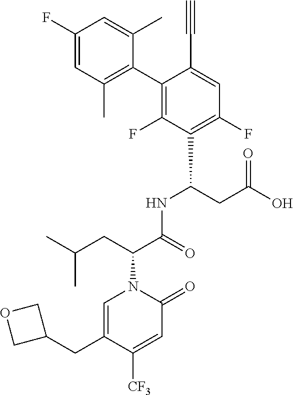 [SMILES: C#Cc1cc(F)c([C@H](CC(=O)O)NC(=O)[C@@H](CC(C)C)n2cc(CC3COC3)c(C(F)(F)F)cc2=O)c(F)c1-c1c(C)cc(F)cc1C] | 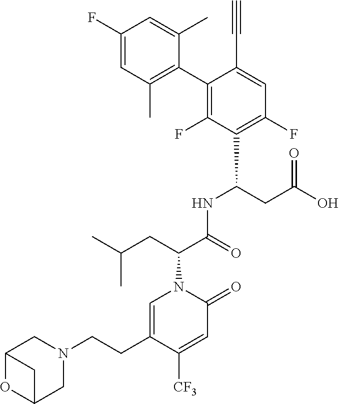 [SMILES: C#Cc1cc(F)c([C@H](CC(=O)O)NC(=O)[C@@H](CC(C)C)n2cc(CCN3CC4CC(C3)O4)c(C(F)(F)F)cc2=O)c(F)c1-c1c(C)cc(F)cc1C] |
| 209 | 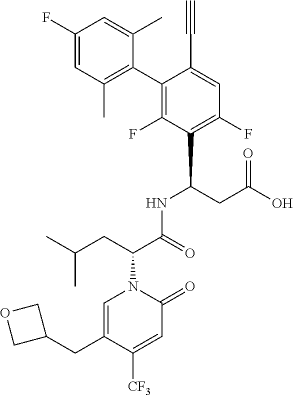 [SMILES: C#Cc1cc(F)c([C@@H](CC(=O)O)NC(=O)[C@@H](CC(C)C)n2cc(CC3COC3)c(C(F)(F)F)cc2=O)c(F)c1-c1c(C)cc(F)cc1C] | 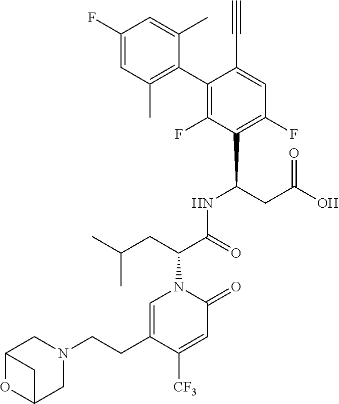 [SMILES: C#Cc1cc(F)c([C@@H](CC(=O)O)NC(=O)[C@@H](CC(C)C)n2cc(CCN3CC4CC(C3)O4)c(C(F)(F)F)cc2=O)c(F)c1-c1c(C)cc(F)cc1C] |
| 210 | 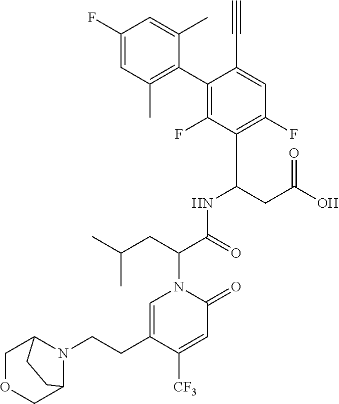 [SMILES: C#Cc1cc(F)c(C(CC(=O)O)NC(=O)C(CC(C)C)n2cc(CCN3C4CCC3COC4)c(C(F)(F)F)cc2=O)c(F)c1-c1c(C)cc(F)cc1C] | 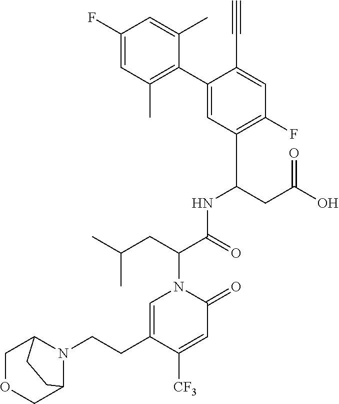 [SMILES: C#Cc1cc(F)c(C(CC(=O)O)NC(=O)C(CC(C)C)n2cc(CCN3C4CCC3COC4)c(C(F)(F)F)cc2=O)cc1-c1c(C)cc(F)cc1C] |
| 211 | 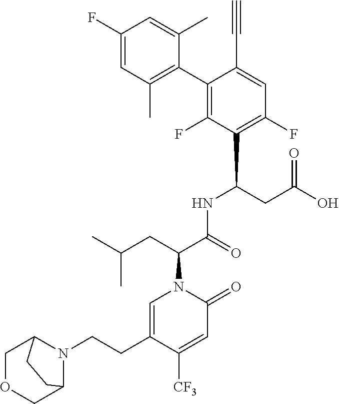 [SMILES: C#Cc1cc(F)c([C@@H](CC(=O)O)NC(=O)[C@H](CC(C)C)n2cc(CCN3C4CCC3COC4)c(C(F)(F)F)cc2=O)c(F)c1-c1c(C)cc(F)cc1C] | 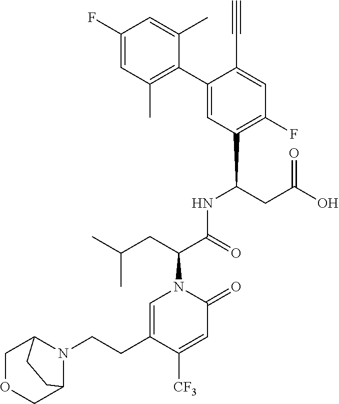 [SMILES: C#Cc1cc(F)c([C@@H](CC(=O)O)NC(=O)[C@H](CC(C)C)n2cc(CCN3C4CCC3COC4)c(C(F)(F)F)cc2=O)cc1-c1c(C)cc(F)cc1C] |
| 212 | 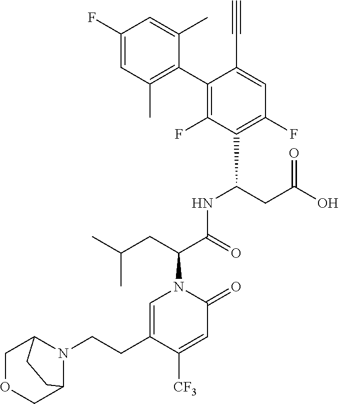 [SMILES: C#Cc1cc(F)c([C@H](CC(=O)O)NC(=O)[C@H](CC(C)C)n2cc(CCN3C4CCC3COC4)c(C(F)(F)F)cc2=O)c(F)c1-c1c(C)cc(F)cc1C] | 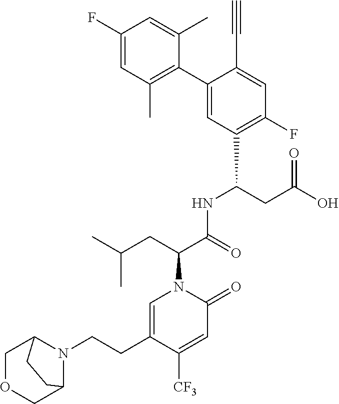 [SMILES: C#Cc1cc(F)c([C@H](CC(=O)O)NC(=O)[C@H](CC(C)C)n2cc(CCN3C4CCC3COC4)c(C(F)(F)F)cc2=O)cc1-c1c(C)cc(F)cc1C] |
| 213 | 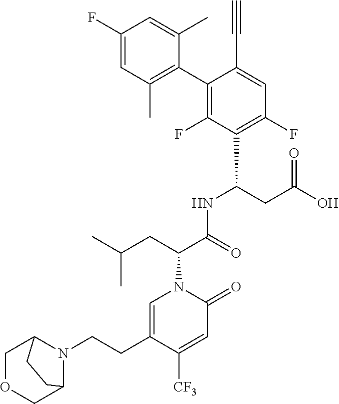 [SMILES: C#Cc1cc(F)c([C@H](CC(=O)O)NC(=O)[C@@H](CC(C)C)n2cc(CCN3C4CCC3COC4)c(C(F)(F)F)cc2=O)c(F)c1-c1c(C)cc(F)cc1C] | 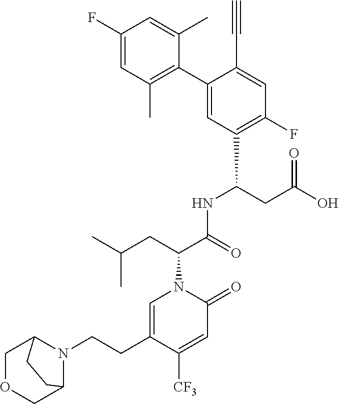 [SMILES: C#Cc1cc(F)c([C@H](CC(=O)O)NC(=O)[C@@H](CC(C)C)n2cc(CCN3C4CCC3COC4)c(C(F)(F)F)cc2=O)cc1-c1c(C)cc(F)cc1C] |
| 214 | 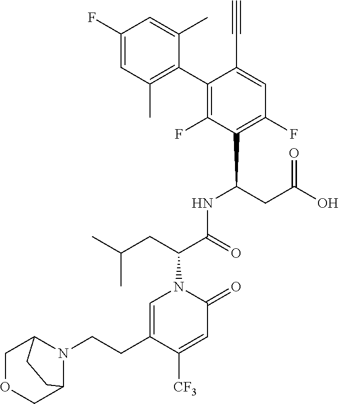 [SMILES: C#Cc1cc(F)c([C@@H](CC(=O)O)NC(=O)[C@@H](CC(C)C)n2cc(CCN3C4CCC3COC4)c(C(F)(F)F)cc2=O)c(F)c1-c1c(C)cc(F)cc1C] | 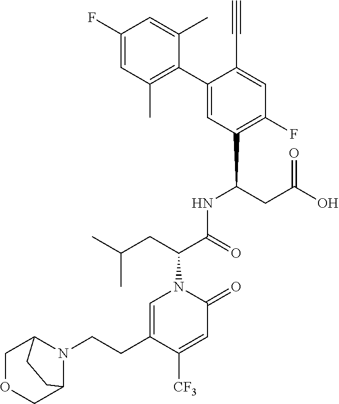 [SMILES: C#Cc1cc(F)c([C@@H](CC(=O)O)NC(=O)[C@@H](CC(C)C)n2cc(CCN3C4CCC3COC4)c(C(F)(F)F)cc2=O)cc1-c1c(C)cc(F)cc1C] |
| 215 | 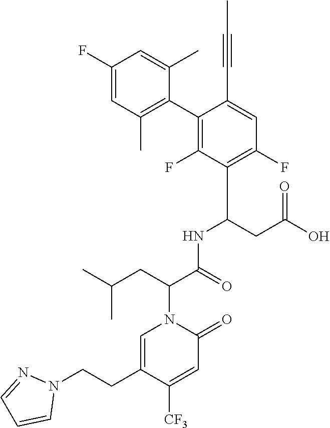 [SMILES: CC#Cc1cc(F)c(C(CC(=O)O)NC(=O)C(CC(C)C)n2cc(CCn3cccn3)c(C(F)(F)F)cc2=O)c(F)c1-c1c(C)cc(F)cc1C] | 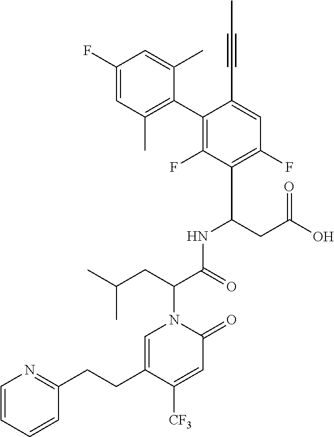 [SMILES: CC#Cc1cc(F)c(C(CC(=O)O)NC(=O)C(CC(C)C)n2cc(CCc3ccccn3)c(C(F)(F)F)cc2=O)c(F)c1-c1c(C)cc(F)cc1C] |
| 216 | 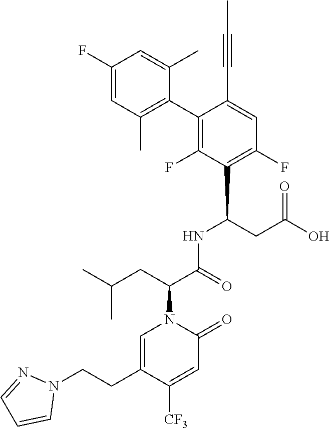 [SMILES: CC#Cc1cc(F)c([C@@H](CC(=O)O)NC(=O)[C@H](CC(C)C)n2cc(CCn3cccn3)c(C(F)(F)F)cc2=O)c(F)c1-c1c(C)cc(F)cc1C] | 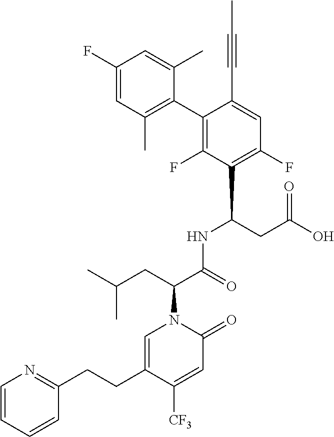 [SMILES: CC#Cc1cc(F)c([C@@H](CC(=O)O)NC(=O)[C@H](CC(C)C)n2cc(CCc3ccccn3)c(C(F)(F)F)cc2=O)c(F)c1-c1c(C)cc(F)cc1C] |
| 217 | 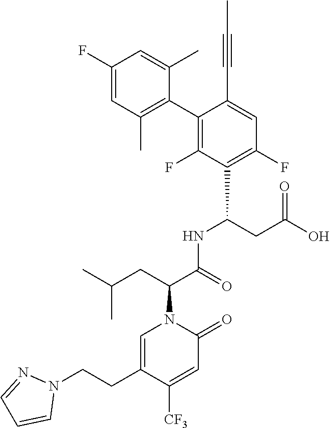 [SMILES: CC#Cc1cc(F)c([C@H](CC(=O)O)NC(=O)[C@H](CC(C)C)n2cc(CCn3cccn3)c(C(F)(F)F)cc2=O)c(F)c1-c1c(C)cc(F)cc1C] | 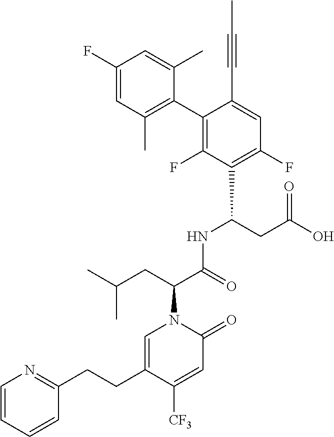 [SMILES: CC#Cc1cc(F)c([C@H](CC(=O)O)NC(=O)[C@H](CC(C)C)n2cc(CCc3ccccn3)c(C(F)(F)F)cc2=O)c(F)c1-c1c(C)cc(F)cc1C] |
| 218 | 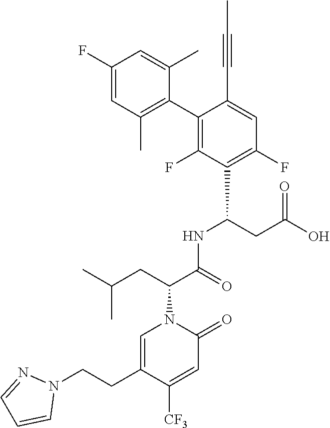 [SMILES: CC#Cc1cc(F)c([C@H](CC(=O)O)NC(=O)[C@@H](CC(C)C)n2cc(CCn3cccn3)c(C(F)(F)F)cc2=O)c(F)c1-c1c(C)cc(F)cc1C] | 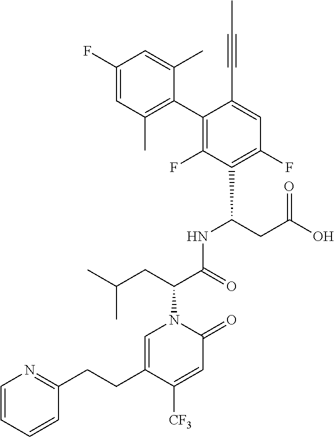 [SMILES: CC#Cc1cc(F)c([C@H](CC(=O)O)NC(=O)[C@@H](CC(C)C)n2cc(CCc3ccccn3)c(C(F)(F)F)cc2=O)c(F)c1-c1c(C)cc(F)cc1C] |
| 219 | 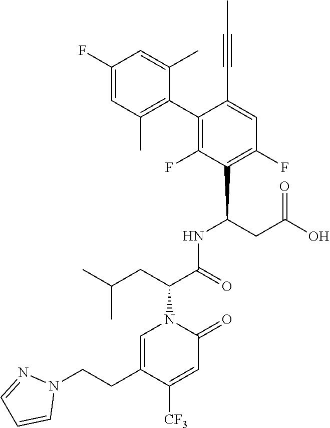 [SMILES: CC#Cc1cc(F)c([C@@H](CC(=O)O)NC(=O)[C@@H](CC(C)C)n2cc(CCn3cccn3)c(C(F)(F)F)cc2=O)c(F)c1-c1c(C)cc(F)cc1C] | 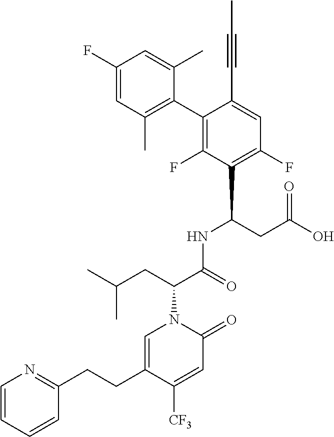 [SMILES: CC#Cc1cc(F)c([C@@H](CC(=O)O)NC(=O)[C@@H](CC(C)C)n2cc(CCc3ccccn3)c(C(F)(F)F)cc2=O)c(F)c1-c1c(C)cc(F)cc1C] |
| 220 | 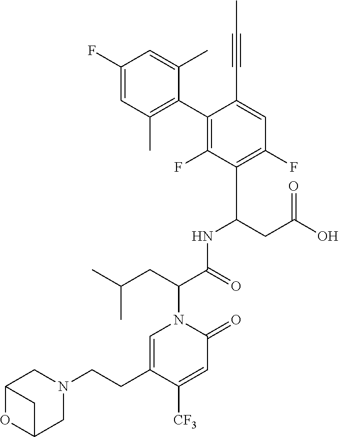 [SMILES: CC#Cc1cc(F)c(C(CC(=O)O)NC(=O)C(CC(C)C)n2cc(CCN3CC4CC(C3)O4)c(C(F)(F)F)cc2=O)c(F)c1-c1c(C)cc(F)cc1C] | 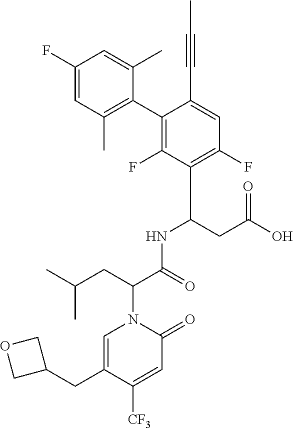 [SMILES: CC#Cc1cc(F)c(C(CC(=O)O)NC(=O)C(CC(C)C)n2cc(CC3COC3)c(C(F)(F)F)cc2=O)c(F)c1-c1c(C)cc(F)cc1C] |
| 221 | 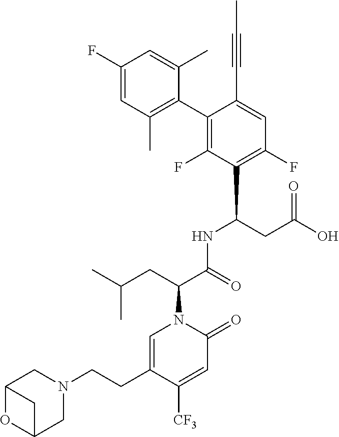 [SMILES: CC#Cc1cc(F)c([C@@H](CC(=O)O)NC(=O)[C@H](CC(C)C)n2cc(CCN3CC4CC(C3)O4)c(C(F)(F)F)cc2=O)c(F)c1-c1c(C)cc(F)cc1C] | 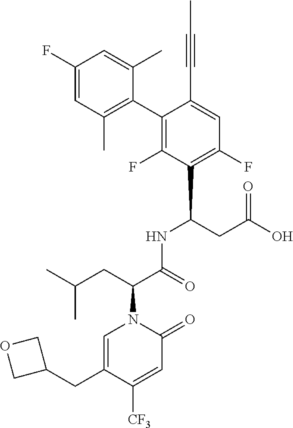 [SMILES: CC#Cc1cc(F)c([C@@H](CC(=O)O)NC(=O)[C@H](CC(C)C)n2cc(CC3COC3)c(C(F)(F)F)cc2=O)c(F)c1-c1c(C)cc(F)cc1C] |
| 222 | 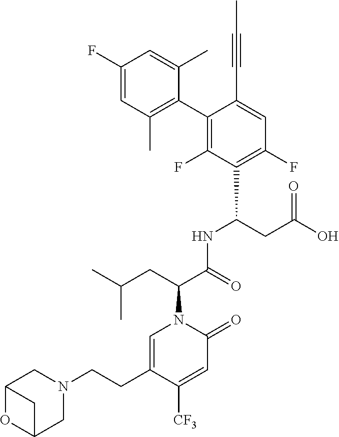 [SMILES: CC#Cc1cc(F)c([C@H](CC(=O)O)NC(=O)[C@H](CC(C)C)n2cc(CCN3CC4CC(C3)O4)c(C(F)(F)F)cc2=O)c(F)c1-c1c(C)cc(F)cc1C] | 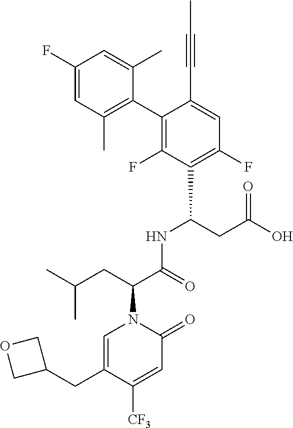 [SMILES: CC#Cc1cc(F)c([C@H](CC(=O)O)NC(=O)[C@H](CC(C)C)n2cc(CC3COC3)c(C(F)(F)F)cc2=O)c(F)c1-c1c(C)cc(F)cc1C] |
| 223 | 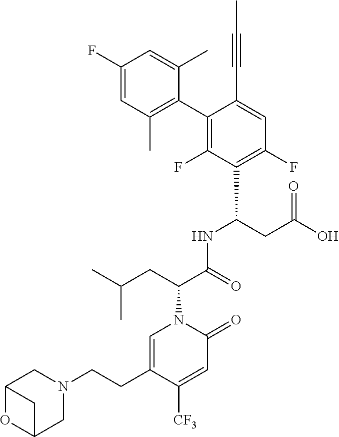 [SMILES: CC#Cc1cc(F)c([C@H](CC(=O)O)NC(=O)[C@@H](CC(C)C)n2cc(CCN3CC4CC(C3)O4)c(C(F)(F)F)cc2=O)c(F)c1-c1c(C)cc(F)cc1C] | 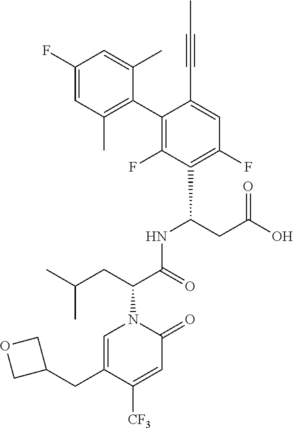 [SMILES: CC#Cc1cc(F)c([C@H](CC(=O)O)NC(=O)[C@@H](CC(C)C)n2cc(CC3COC3)c(C(F)(F)F)cc2=O)c(F)c1-c1c(C)cc(F)cc1C] |
| 224 | 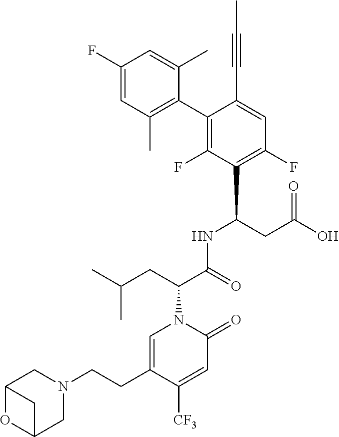 [SMILES: CC#Cc1cc(F)c([C@@H](CC(=O)O)NC(=O)[C@@H](CC(C)C)n2cc(CCN3CC4CC(C3)O4)c(C(F)(F)F)cc2=O)c(F)c1-c1c(C)cc(F)cc1C] | 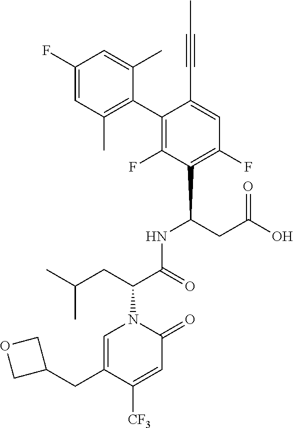 [SMILES: CC#Cc1cc(F)c([C@@H](CC(=O)O)NC(=O)[C@@H](CC(C)C)n2cc(CC3COC3)c(C(F)(F)F)cc2=O)c(F)c1-c1c(C)cc(F)cc1C] |
| 225 | 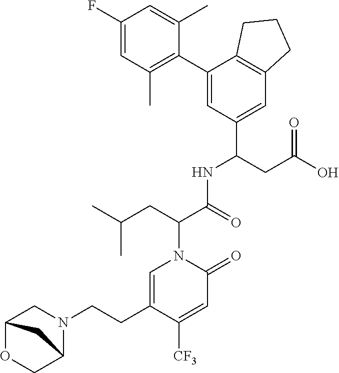 [SMILES: Cc1cc(F)cc(C)c1-c1cc(C(CC(=O)O)NC(=O)C(CC(C)C)n2cc(CCN3C[C@@H]4C[C@H]3CO4)c(C(F)(F)F)cc2=O)cc2c1CCC2] | 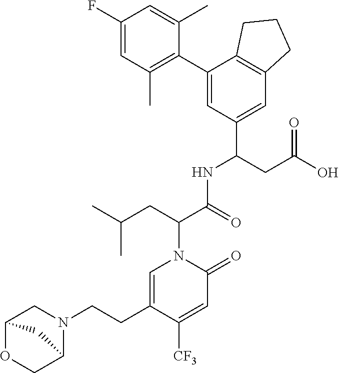 [SMILES: Cc1cc(F)cc(C)c1-c1cc(C(CC(=O)O)NC(=O)C(CC(C)C)n2cc(CCN3C[C@H]4C[C@@H]3CO4)c(C(F)(F)F)cc2=O)cc2c1CCC2] |
| 226 | 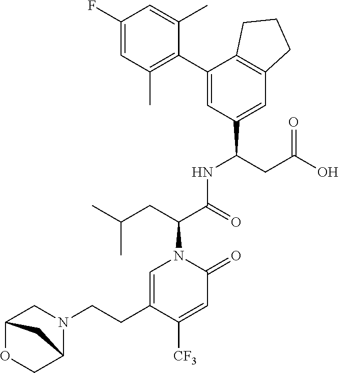 [SMILES: Cc1cc(F)cc(C)c1-c1cc([C@@H](CC(=O)O)NC(=O)[C@H](CC(C)C)n2cc(CCN3C[C@@H]4C[C@H]3CO4)c(C(F)(F)F)cc2=O)cc2c1CCC2] | 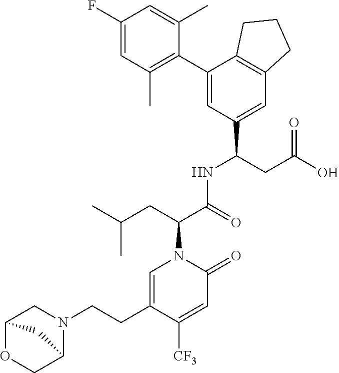 [SMILES: Cc1cc(F)cc(C)c1-c1cc([C@@H](CC(=O)O)NC(=O)[C@H](CC(C)C)n2cc(CCN3C[C@H]4C[C@@H]3CO4)c(C(F)(F)F)cc2=O)cc2c1CCC2] |
| 227 | 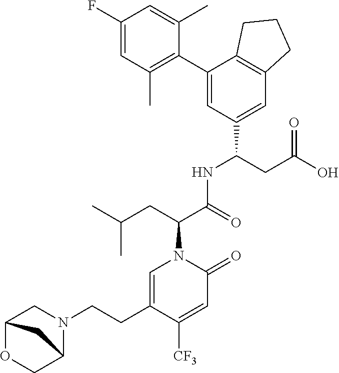 [SMILES: Cc1cc(F)cc(C)c1-c1cc([C@H](CC(=O)O)NC(=O)[C@H](CC(C)C)n2cc(CCN3C[C@@H]4C[C@H]3CO4)c(C(F)(F)F)cc2=O)cc2c1CCC2] | 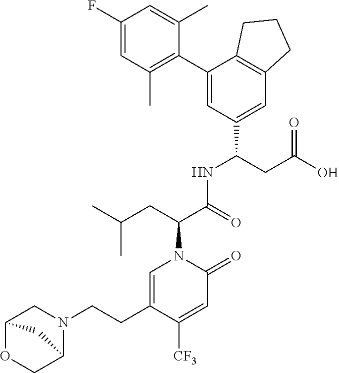 [SMILES: Cc1cc(F)cc(C)c1-c1cc([C@H](CC(=O)O)NC(=O)[C@H](CC(C)C)n2cc(CCN3C[C@H]4C[C@@H]3CO4)c(C(F)(F)F)cc2=O)cc2c1CCC2] |
| 228 | 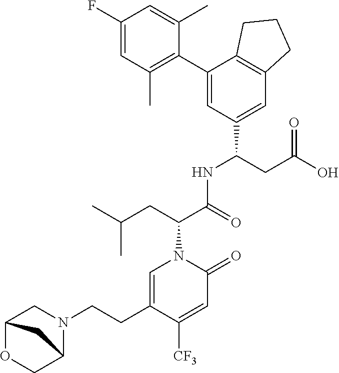 [SMILES: Cc1cc(F)cc(C)c1-c1cc([C@H](CC(=O)O)NC(=O)[C@@H](CC(C)C)n2cc(CCN3C[C@@H]4C[C@H]3CO4)c(C(F)(F)F)cc2=O)cc2c1CCC2] | 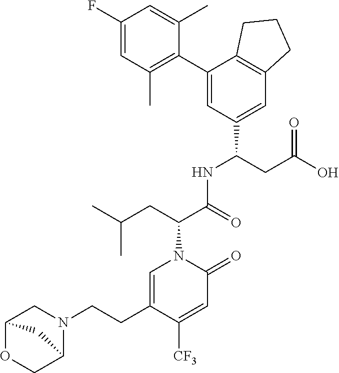 [SMILES: Cc1cc(F)cc(C)c1-c1cc([C@H](CC(=O)O)NC(=O)[C@@H](CC(C)C)n2cc(CCN3C[C@H]4C[C@@H]3CO4)c(C(F)(F)F)cc2=O)cc2c1CCC2] |
| 229 | 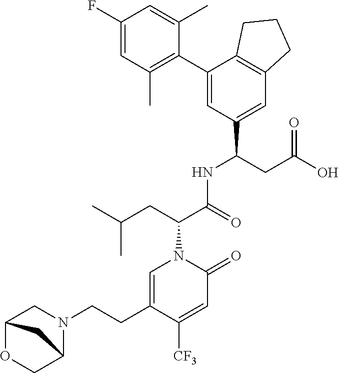 [SMILES: Cc1cc(F)cc(C)c1-c1cc([C@@H](CC(=O)O)NC(=O)[C@@H](CC(C)C)n2cc(CCN3C[C@@H]4C[C@H]3CO4)c(C(F)(F)F)cc2=O)cc2c1CCC2] | 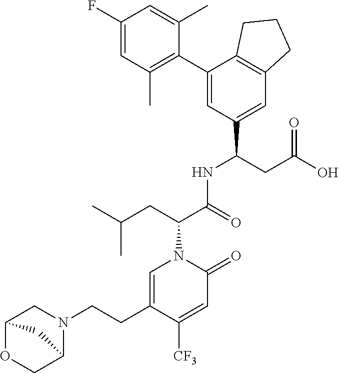 [SMILES: Cc1cc(F)cc(C)c1-c1cc([C@@H](CC(=O)O)NC(=O)[C@@H](CC(C)C)n2cc(CCN3C[C@H]4C[C@@H]3CO4)c(C(F)(F)F)cc2=O)cc2c1CCC2] |
| 230 | 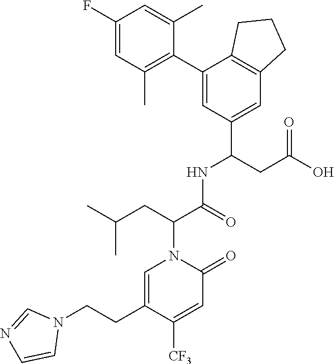 [SMILES: Cc1cc(F)cc(C)c1-c1cc(C(CC(=O)O)NC(=O)C(CC(C)C)n2cc(CCn3ccnc3)c(C(F)(F)F)cc2=O)cc2c1CCC2] | 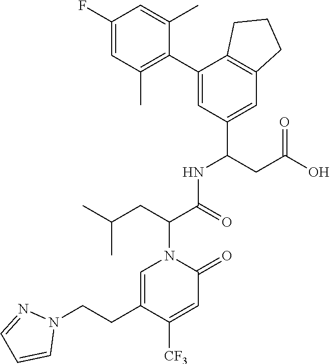 [SMILES: Cc1cc(F)cc(C)c1-c1cc(C(CC(=O)O)NC(=O)C(CC(C)C)n2cc(CCn3cccn3)c(C(F)(F)F)cc2=O)cc2c1CCC2] |
| 231 | 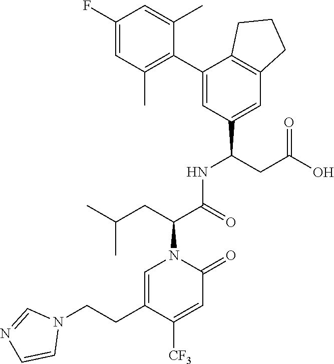 [SMILES: Cc1cc(F)cc(C)c1-c1cc([C@@H](CC(=O)O)NC(=O)[C@H](CC(C)C)n2cc(CCn3ccnc3)c(C(F)(F)F)cc2=O)cc2c1CCC2] | 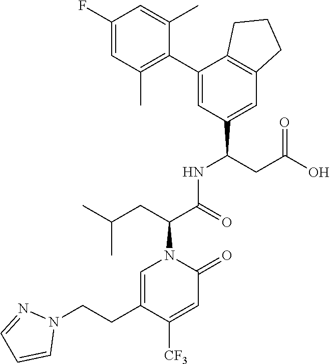 [SMILES: Cc1cc(F)cc(C)c1-c1cc([C@@H](CC(=O)O)NC(=O)[C@H](CC(C)C)n2cc(CCn3cccn3)c(C(F)(F)F)cc2=O)cc2c1CCC2] |
| 232 | 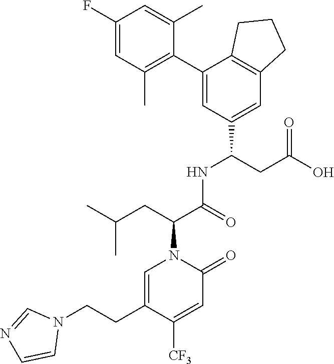 [SMILES: Cc1cc(F)cc(C)c1-c1cc([C@H](CC(=O)O)NC(=O)[C@H](CC(C)C)n2cc(CCn3ccnc3)c(C(F)(F)F)cc2=O)cc2c1CCC2] | 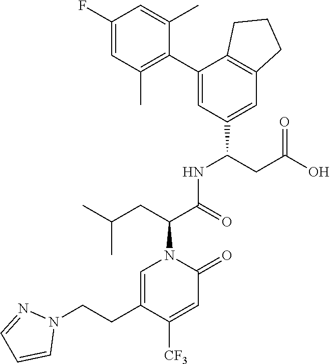 [SMILES: Cc1cc(F)cc(C)c1-c1cc([C@H](CC(=O)O)NC(=O)[C@H](CC(C)C)n2cc(CCn3cccn3)c(C(F)(F)F)cc2=O)cc2c1CCC2] |
| 233 | 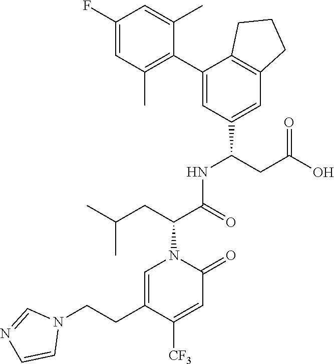 [SMILES: Cc1cc(F)cc(C)c1-c1cc([C@H](CC(=O)O)NC(=O)[C@@H](CC(C)C)n2cc(CCn3ccnc3)c(C(F)(F)F)cc2=O)cc2c1CCC2] | 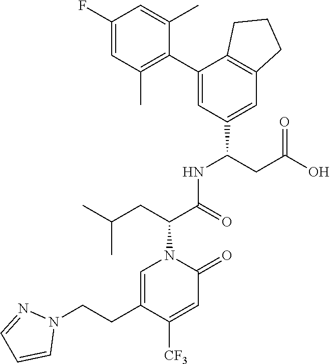 [SMILES: Cc1cc(F)cc(C)c1-c1cc([C@H](CC(=O)O)NC(=O)[C@@H](CC(C)C)n2cc(CCn3cccn3)c(C(F)(F)F)cc2=O)cc2c1CCC2] |
| 234 | 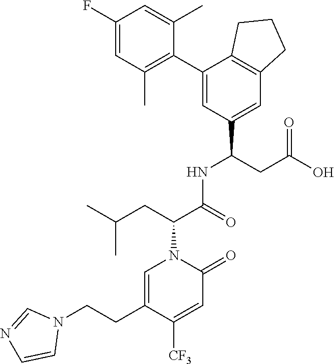 [SMILES: Cc1cc(F)cc(C)c1-c1cc([C@@H](CC(=O)O)NC(=O)[C@@H](CC(C)C)n2cc(CCn3ccnc3)c(C(F)(F)F)cc2=O)cc2c1CCC2] | 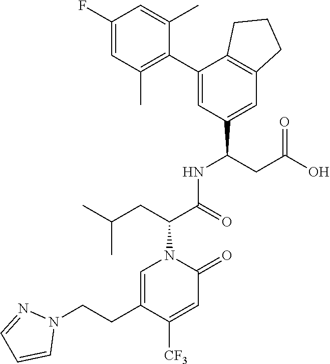 [SMILES: Cc1cc(F)cc(C)c1-c1cc([C@@H](CC(=O)O)NC(=O)[C@@H](CC(C)C)n2cc(CCn3cccn3)c(C(F)(F)F)cc2=O)cc2c1CCC2] |
| 235 | 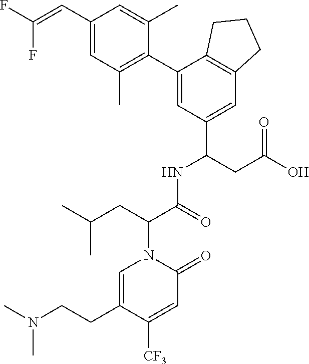 [SMILES: Cc1cc(C=C(F)F)cc(C)c1-c1cc(C(CC(=O)O)NC(=O)C(CC(C)C)n2cc(CCN(C)C)c(C(F)(F)F)cc2=O)cc2c1CCC2] | 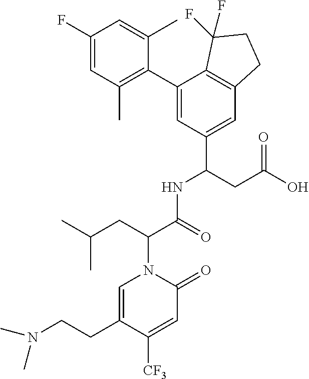 [SMILES: Cc1cc(F)cc(C)c1-c1cc(C(CC(=O)O)NC(=O)C(CC(C)C)n2cc(CCN(C)C)c(C(F)(F)F)cc2=O)cc2c1C(F)(F)CC2] |
| 236 | 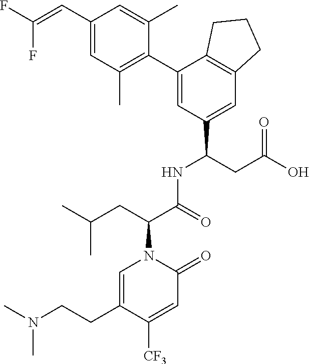 [SMILES: Cc1cc(C=C(F)F)cc(C)c1-c1cc([C@@H](CC(=O)O)NC(=O)[C@H](CC(C)C)n2cc(CCN(C)C)c(C(F)(F)F)cc2=O)cc2c1CCC2] | 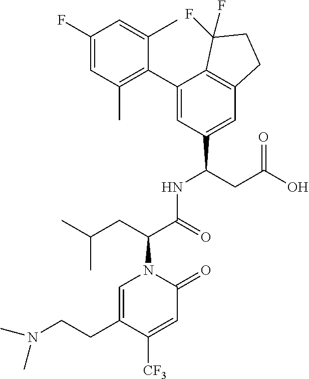 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)cc2c1C(F)(F)CC2] |
| 237 | 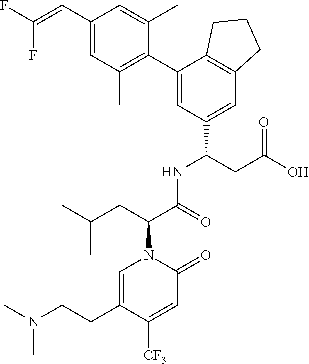 [SMILES: Cc1cc(C=C(F)F)cc(C)c1-c1cc([C@H](CC(=O)O)NC(=O)[C@H](CC(C)C)n2cc(CCN(C)C)c(C(F)(F)F)cc2=O)cc2c1CCC2] | 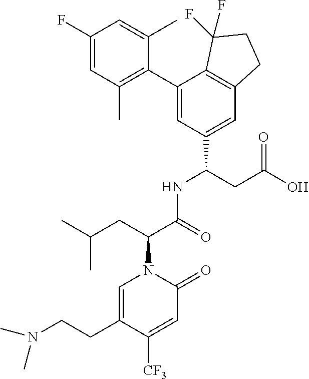 [SMILES: Cc1cc(F)cc(C)c1-c1cc([C@H](CC(=O)O)NC(=O)[C@H](CC(C)C)n2cc(CCN(C)C)c(C(F)(F)F)cc2=O)cc2c1C(F)(F)CC2] |
| 238 | 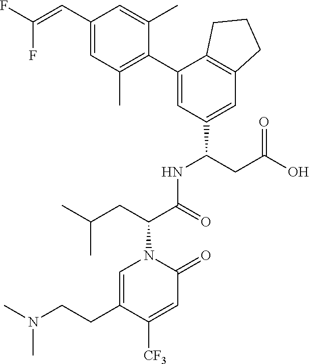 [SMILES: Cc1cc(C=C(F)F)cc(C)c1-c1cc([C@H](CC(=O)O)NC(=O)[C@@H](CC(C)C)n2cc(CCN(C)C)c(C(F)(F)F)cc2=O)cc2c1CCC2] | 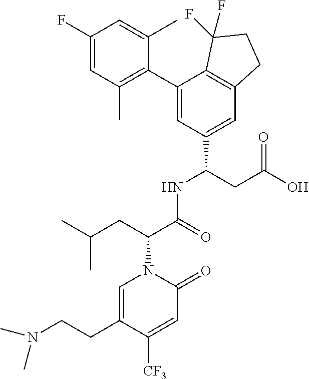 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cc2c1C(F)(F)CC2] |
| 239 | 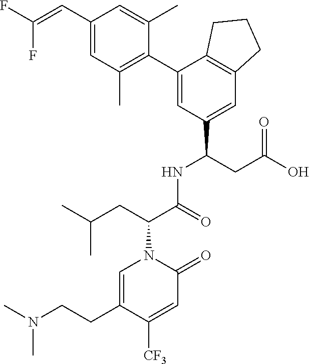 [SMILES: Cc1cc(C=C(F)F)cc(C)c1-c1cc([C@@H](CC(=O)O)NC(=O)[C@@H](CC(C)C)n2cc(CCN(C)C)c(C(F)(F)F)cc2=O)cc2c1CCC2] | 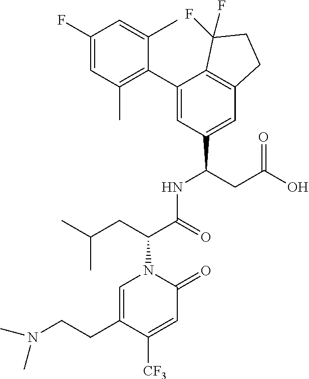 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)cc2c1C(F)(F)CC2] |
| 240 | 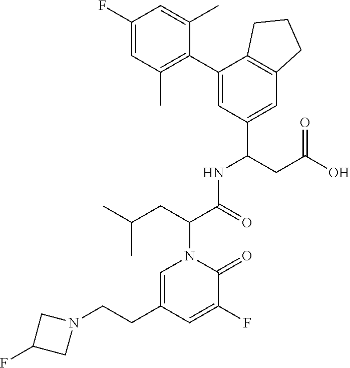 [SMILES: Cc1cc(F)cc(C)c1-c1cc(C(CC(=O)O)NC(=O)C(CC(C)C)n2cc(CCN3CC(F)C3)cc(F)c2=O)cc2c1CCC2] | 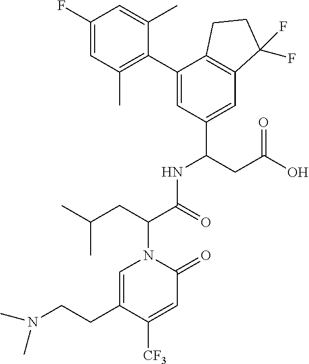 [SMILES: Cc1cc(F)cc(C)c1-c1cc(C(CC(=O)O)NC(=O)C(CC(C)C)n2cc(CCN(C)C)c(C(F)(F)F)cc2=O)cc2c1CCC2(F)F] |
| 241 | 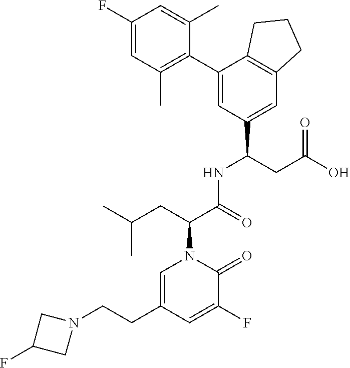 [SMILES: Cc1cc(F)cc(C)c1-c1cc([C@@H](CC(=O)O)NC(=O)[C@H](CC(C)C)n2cc(CCN3CC(F)C3)cc(F)c2=O)cc2c1CCC2] | 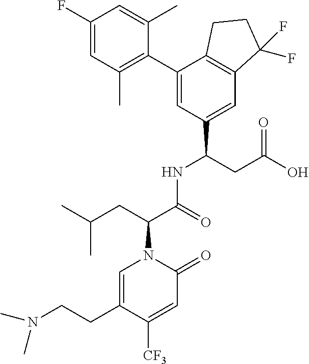 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)cc2c1CCC2(F)F] |
| 242 | 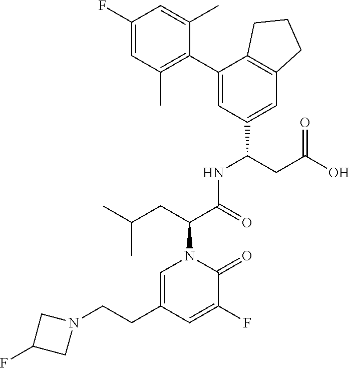 [SMILES: Cc1cc(F)cc(C)c1-c1cc([C@H](CC(=O)O)NC(=O)[C@H](CC(C)C)n2cc(CCN3CC(F)C3)cc(F)c2=O)cc2c1CCC2] | 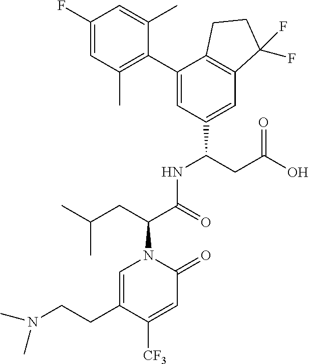 [SMILES: Cc1cc(F)cc(C)c1-c1cc([C@H](CC(=O)O)NC(=O)[C@H](CC(C)C)n2cc(CCN(C)C)c(C(F)(F)F)cc2=O)cc2c1CCC2(F)F] |
| 243 | 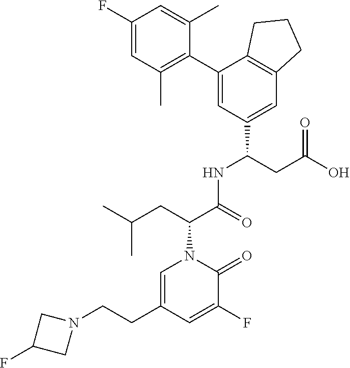 [SMILES: Cc1cc(F)cc(C)c1-c1cc([C@H](CC(=O)O)NC(=O)[C@@H](CC(C)C)n2cc(CCN3CC(F)C3)cc(F)c2=O)cc2c1CCC2] | 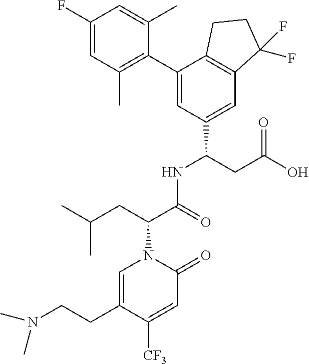 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)cc2c1CCC2(F)F] |
| 244 | 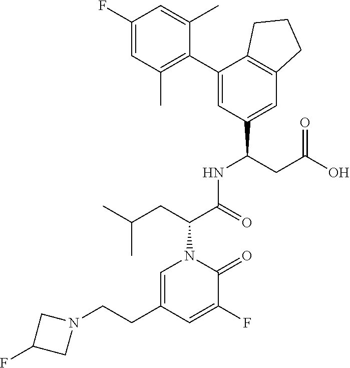 [SMILES: Cc1cc(F)cc(C)c1-c1cc([C@@H](CC(=O)O)NC(=O)[C@@H](CC(C)C)n2cc(CCN3CC(F)C3)cc(F)c2=O)cc2c1CCC2] | 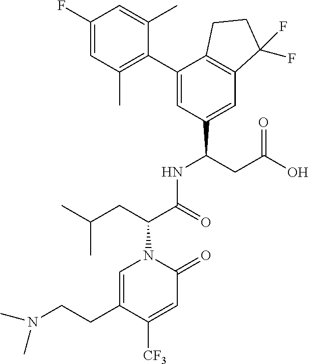 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)cc2c1CCC2(F)F] |
| 245 | 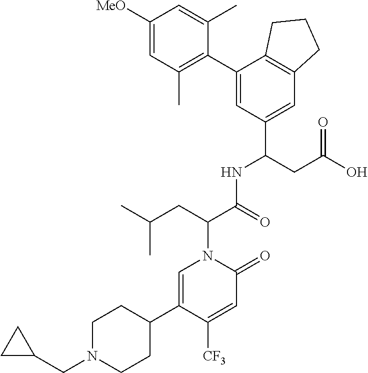 [SMILES: COc1cc(C)c(-c2cc(C(CC(=O)O)NC(=O)C(CC(C)C)n3cc(C4CCN(CC5CC5)CC4)c(C(F)(F)F)cc3=O)cc3c2CCC3)c(C)c1] | 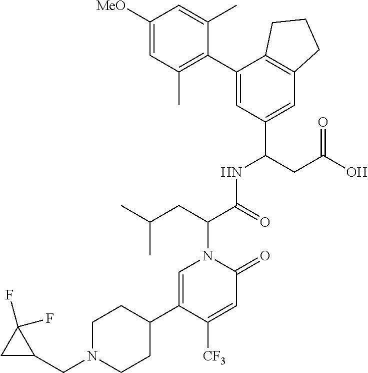 [SMILES: COc1cc(C)c(-c2cc(C(CC(=O)O)NC(=O)C(CC(C)C)n3cc(C4CCN(CC5CC5(F)F)CC4)c(C(F)(F)F)cc3=O)cc3c2CCC3)c(C)c1] |
| 246 | 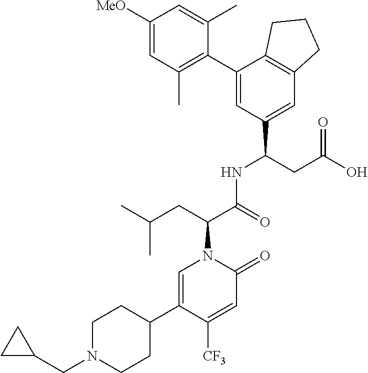 [SMILES: COc1cc(C)c(-c2cc([C@@H](CC(=O)O)NC(=O)[C@H](CC(C)C)n3cc(C4CCN(CC5CC5)CC4)c(C(F)(F)F)cc3=O)cc3c2CCC3)c(C)c1] | 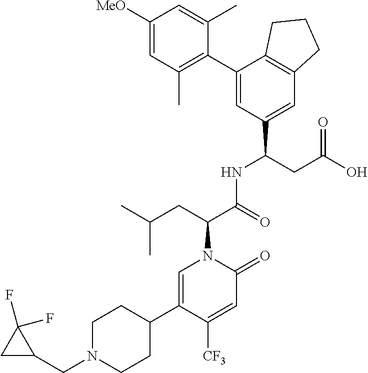 [SMILES: COc1cc(C)c(-c2cc([C@@H](CC(=O)O)NC(=O)[C@H](CC(C)C)n3cc(C4CCN(CC5CC5(F)F)CC4)c(C(F)(F)F)cc3=O)cc3c2CCC3)c(C)c1] |
| 247 | 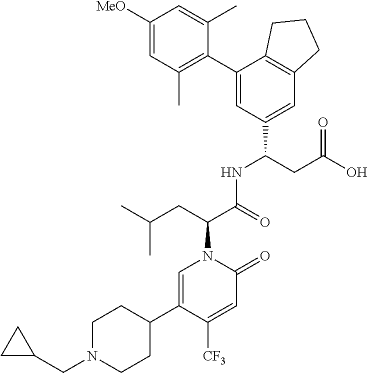 [SMILES: COc1cc(C)c(-c2cc([C@H](CC(=O)O)NC(=O)[C@H](CC(C)C)n3cc(C4CCN(CC5CC5)CC4)c(C(F)(F)F)cc3=O)cc3c2CCC3)c(C)c1] | 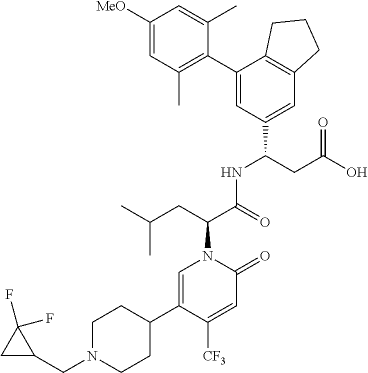 [SMILES: COc1cc(C)c(-c2cc([C@H](CC(=O)O)NC(=O)[C@H](CC(C)C)n3cc(C4CCN(CC5CC5(F)F)CC4)c(C(F)(F)F)cc3=O)cc3c2CCC3)c(C)c1] |
| 248 | 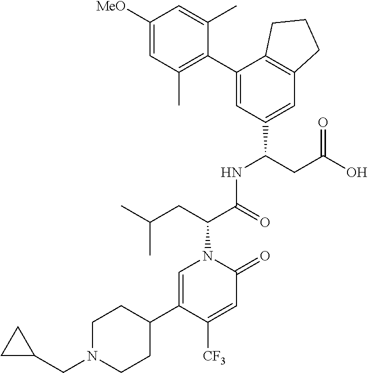 [SMILES: COc1cc(C)c(-c2cc([C@H](CC(=O)O)NC(=O)[C@@H](CC(C)C)n3cc(C4CCN(CC5CC5)CC4)c(C(F)(F)F)cc3=O)cc3c2CCC3)c(C)c1] | 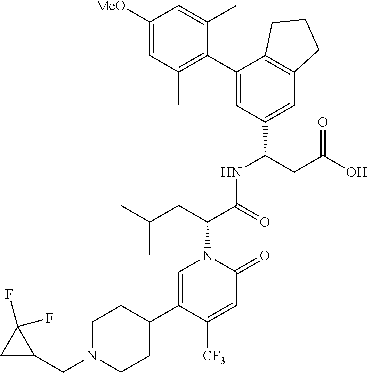 [SMILES: COc1cc(C)c(-c2cc([C@H](CC(=O)O)NC(=O)[C@@H](CC(C)C)n3cc(C4CCN(CC5CC5(F)F)CC4)c(C(F)(F)F)cc3=O)cc3c2CCC3)c(C)c1] |
| 249 | 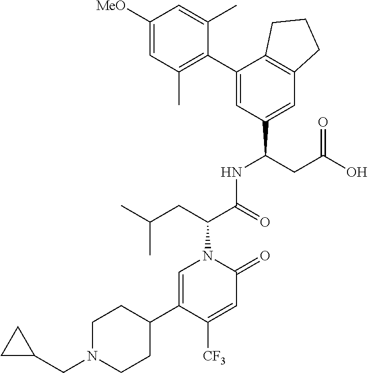 [SMILES: COc1cc(C)c(-c2cc([C@@H](CC(=O)O)NC(=O)[C@@H](CC(C)C)n3cc(C4CCN(CC5CC5)CC4)c(C(F)(F)F)cc3=O)cc3c2CCC3)c(C)c1] | 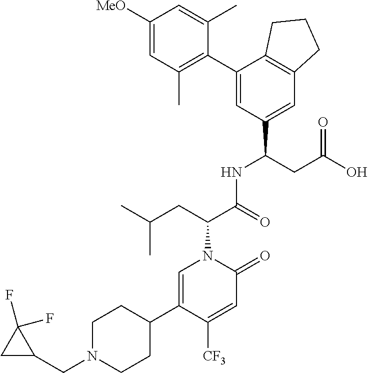 [SMILES: COc1cc(C)c(-c2cc([C@@H](CC(=O)O)NC(=O)[C@@H](CC(C)C)n3cc(C4CCN(CC5CC5(F)F)CC4)c(C(F)(F)F)cc3=O)cc3c2CCC3)c(C)c1] |
| 250 | | |
| 251 | | |
| 252 | | |
| 253 | | |
| 254 | | |
| 255 | 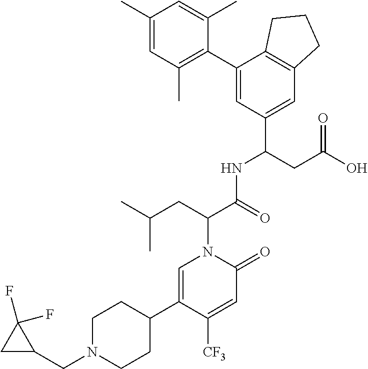 [SMILES: Cc1cc(C)c(-c2cc(C(CC(=O)O)NC(=O)C(CC(C)C)n3cc(C4CCN(CC5CC5(F)F)CC4)c(C(F)(F)F)cc3=O)cc3c2CCC3)c(C)c1] | 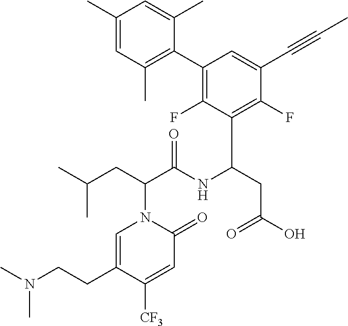 [SMILES: CC#Cc1cc(-c2c(C)cc(C)cc2C)c(F)c(C(CC(=O)O)NC(=O)C(CC(C)C)n2cc(CCN(C)C)c(C(F)(F)F)cc2=O)c1F] |
| 256 | 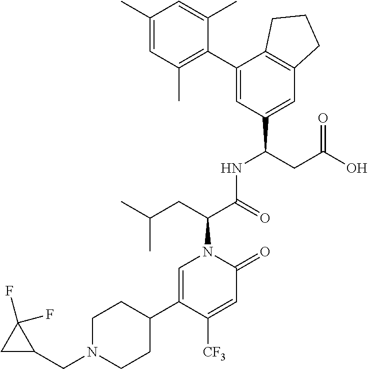 [SMILES: Cc1cc(C)c(-c2cc([C@@H](CC(=O)O)NC(=O)[C@H](CC(C)C)n3cc(C4CCN(CC5CC5(F)F)CC4)c(C(F)(F)F)cc3=O)cc3c2CCC3)c(C)c1] | 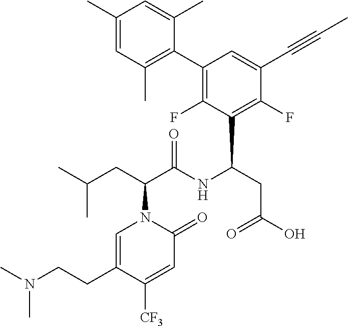 [SMILES: CC#Cc1cc(-c2c(C)cc(C)cc2C)c(F)c([C@@H](CC(=O)O)NC(=O)[C@H](CC(C)C)n2cc(CCN(C)C)c(C(F)(F)F)cc2=O)c1F] |
| 257 | 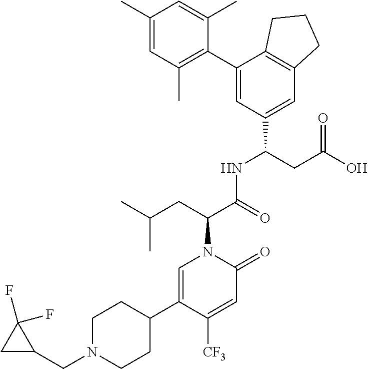 [SMILES: Cc1cc(C)c(-c2cc([C@H](CC(=O)O)NC(=O)[C@H](CC(C)C)n3cc(C4CCN(CC5CC5(F)F)CC4)c(C(F)(F)F)cc3=O)cc3c2CCC3)c(C)c1] | 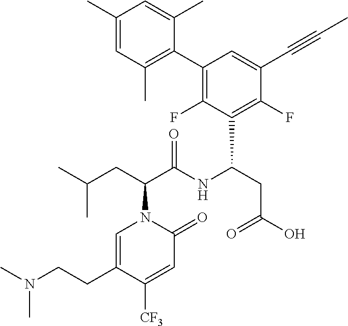 [SMILES: CC#Cc1cc(-c2c(C)cc(C)cc2C)c(F)c([C@H](CC(=O)O)NC(=O)[C@H](CC(C)C)n2cc(CCN(C)C)c(C(F)(F)F)cc2=O)c1F] |
| 258 | 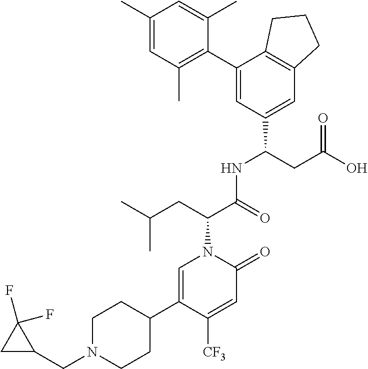 [SMILES: Cc1cc(C)c(-c2cc([C@H](CC(=O)O)NC(=O)[C@@H](CC(C)C)n3cc(C4CCN(CC5CC5(F)F)CC4)c(C(F)(F)F)cc3=O)cc3c2CCC3)c(C)c1] | 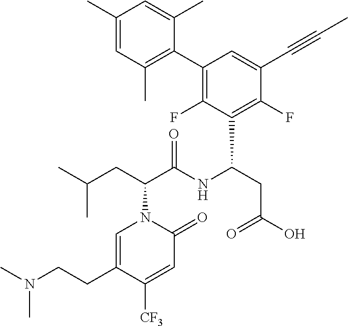 [SMILES: CC#Cc1cc(-c2c(C)cc(C)cc2C)c(F)c([C@H](CC(=O)O)NC(=O)[C@@H](CC(C)C)n2cc(CCN(C)C)c(C(F)(F)F)cc2=O)c1F] |
| 259 | 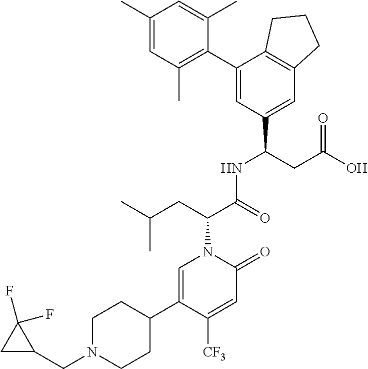 [SMILES: Cc1cc(C)c(-c2cc([C@@H](CC(=O)O)NC(=O)[C@@H](CC(C)C)n3cc(C4CCN(CC5CC5(F)F)CC4)c(C(F)(F)F)cc3=O)cc3c2CCC3)c(C)c1] | 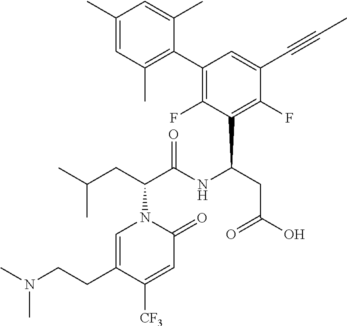 [SMILES: CC#Cc1cc(-c2c(C)cc(C)cc2C)c(F)c([C@@H](CC(=O)O)NC(=O)[C@@H](CC(C)C)n2cc(CCN(C)C)c(C(F)(F)F)cc2=O)c1F] |
| 260 | 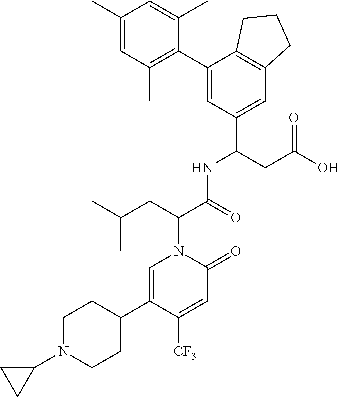 [SMILES: Cc1cc(C)c(-c2cc(C(CC(=O)O)NC(=O)C(CC(C)C)n3cc(C4CCN(C5CC5)CC4)c(C(F)(F)F)cc3=O)cc3c2CCC3)c(C)c1] | 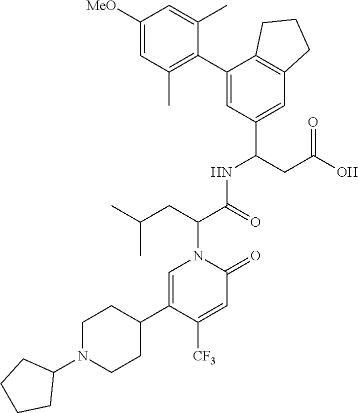 [SMILES: COc1cc(C)c(-c2cc(C(CC(=O)O)NC(=O)C(CC(C)C)n3cc(C4CCN(C5CCCC5)CC4)c(C(F)(F)F)cc3=O)cc3c2CCC3)c(C)c1] |
| 261 | 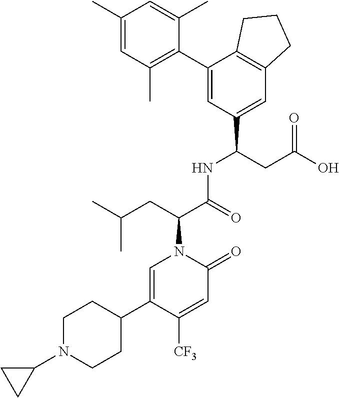 [SMILES: Cc1cc(C)c(-c2cc([C@@H](CC(=O)O)NC(=O)[C@H](CC(C)C)n3cc(C4CCN(C5CC5)CC4)c(C(F)(F)F)cc3=O)cc3c2CCC3)c(C)c1] | 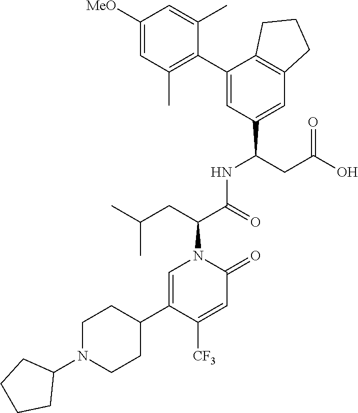 [SMILES: COc1cc(C)c(-c2cc([C@@H](CC(=O)O)NC(=O)[C@H](CC(C)C)n3cc(C4CCN(C5CCCC5)CC4)c(C(F)(F)F)cc3=O)cc3c2CCC3)c(C)c1] |
| 262 | 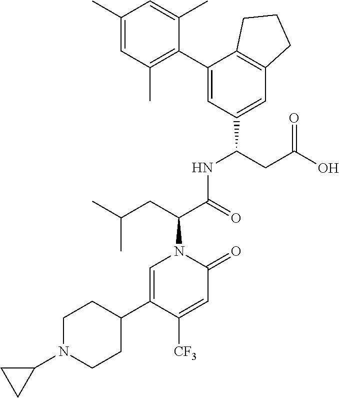 [SMILES: Cc1cc(C)c(-c2cc([C@H](CC(=O)O)NC(=O)[C@H](CC(C)C)n3cc(C4CCN(C5CC5)CC4)c(C(F)(F)F)cc3=O)cc3c2CCC3)c(C)c1] | 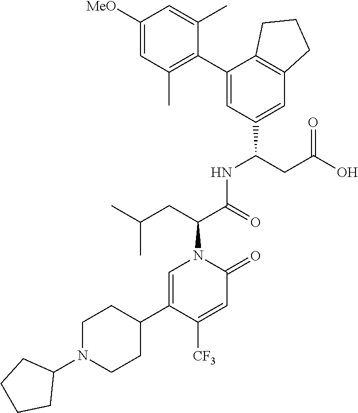 [SMILES: COc1cc(C)c(-c2cc([C@H](CC(=O)O)NC(=O)[C@H](CC(C)C)n3cc(C4CCN(C5CCCC5)CC4)c(C(F)(F)F)cc3=O)cc3c2CCC3)c(C)c1] |
| 263 | 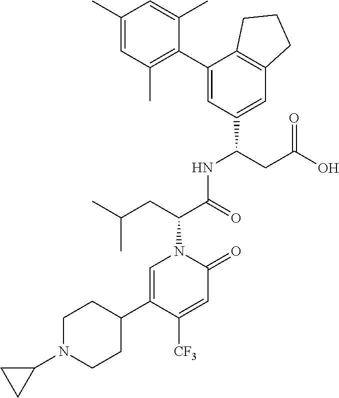 [SMILES: Cc1cc(C)c(-c2cc([C@H](CC(=O)O)NC(=O)[C@@H](CC(C)C)n3cc(C4CCN(C5CC5)CC4)c(C(F)(F)F)cc3=O)cc3c2CCC3)c(C)c1] | 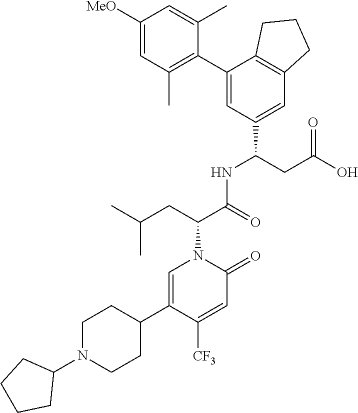 [SMILES: COc1cc(C)c(-c2cc([C@H](CC(=O)O)NC(=O)[C@@H](CC(C)C)n3cc(C4CCN(C5CCCC5)CC4)c(C(F)(F)F)cc3=O)cc3c2CCC3)c(C)c1] |
| 264 | 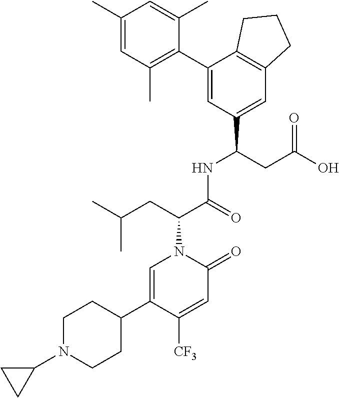 [SMILES: Cc1cc(C)c(-c2cc([C@@H](CC(=O)O)NC(=O)[C@@H](CC(C)C)n3cc(C4CCN(C5CC5)CC4)c(C(F)(F)F)cc3=O)cc3c2CCC3)c(C)c1] | 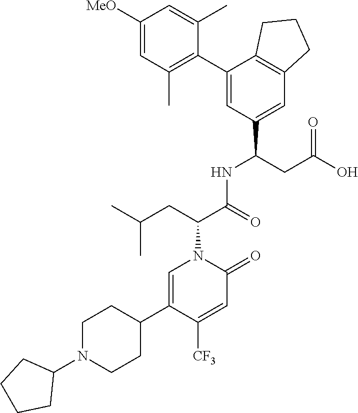 [SMILES: COc1cc(C)c(-c2cc([C@@H](CC(=O)O)NC(=O)[C@@H](CC(C)C)n3cc(C4CCN(C5CCCC5)CC4)c(C(F)(F)F)cc3=O)cc3c2CCC3)c(C)c1] |
| 265 | 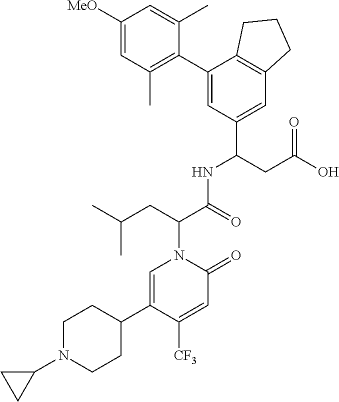 [SMILES: COc1cc(C)c(-c2cc(C(CC(=O)O)NC(=O)C(CC(C)C)n3cc(C4CCN(C5CC5)CC4)c(C(F)(F)F)cc3=O)cc3c2CCC3)c(C)c1] | 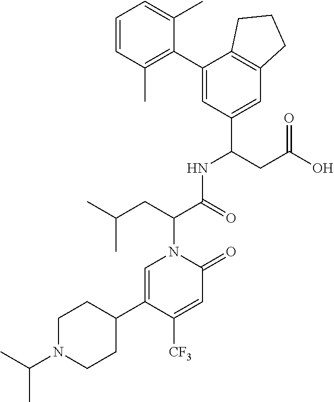 [SMILES: Cc1cccc(C)c1-c1cc(C(CC(=O)O)NC(=O)C(CC(C)C)n2cc(C3CCN(C(C)C)CC3)c(C(F)(F)F)cc2=O)cc2c1CCC2] |
| 266 | 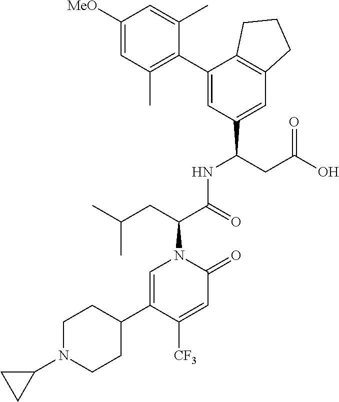 [SMILES: COc1cc(C)c(-c2cc([C@@H](CC(=O)O)NC(=O)[C@H](CC(C)C)n3cc(C4CCN(C5CC5)CC4)c(C(F)(F)F)cc3=O)cc3c2CCC3)c(C)c1] | 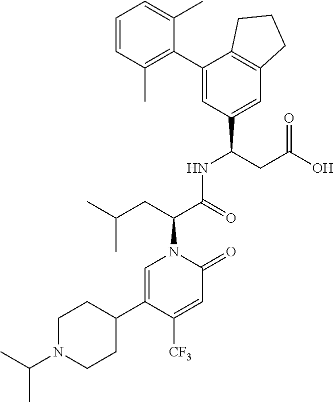 [SMILES: Cc1cccc(C)c1-c1cc([C@@H](CC(=O)O)NC(=O)[C@H](CC(C)C)n2cc(C3CCN(C(C)C)CC3)c(C(F)(F)F)cc2=O)cc2c1CCC2] |
| 267 | 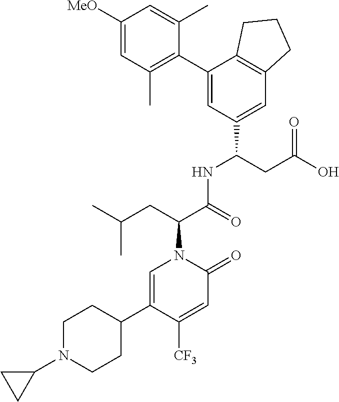 [SMILES: COc1cc(C)c(-c2cc([C@H](CC(=O)O)NC(=O)[C@H](CC(C)C)n3cc(C4CCN(C5CC5)CC4)c(C(F)(F)F)cc3=O)cc3c2CCC3)c(C)c1] | 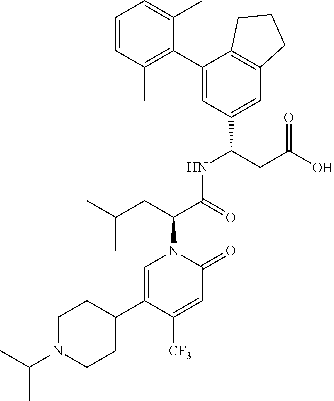 [SMILES: Cc1cccc(C)c1-c1cc([C@H](CC(=O)O)NC(=O)[C@H](CC(C)C)n2cc(C3CCN(C(C)C)CC3)c(C(F)(F)F)cc2=O)cc2c1CCC2] |
| 268 | 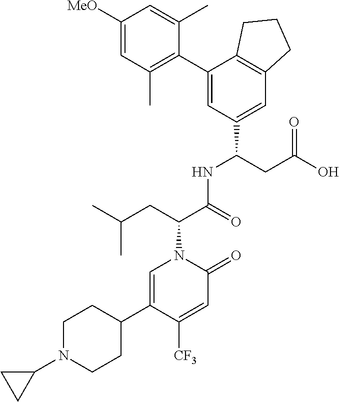 [SMILES: COc1cc(C)c(-c2cc([C@H](CC(=O)O)NC(=O)[C@@H](CC(C)C)n3cc(C4CCN(C5CC5)CC4)c(C(F)(F)F)cc3=O)cc3c2CCC3)c(C)c1] | 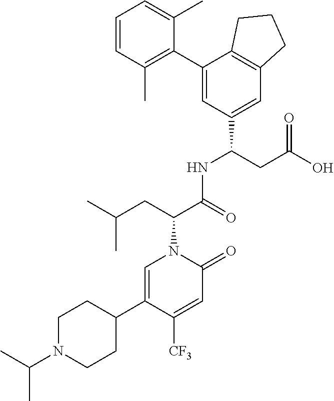 [SMILES: Cc1cccc(C)c1-c1cc([C@H](CC(=O)O)NC(=O)[C@@H](CC(C)C)n2cc(C3CCN(C(C)C)CC3)c(C(F)(F)F)cc2=O)cc2c1CCC2] |
| 269 | 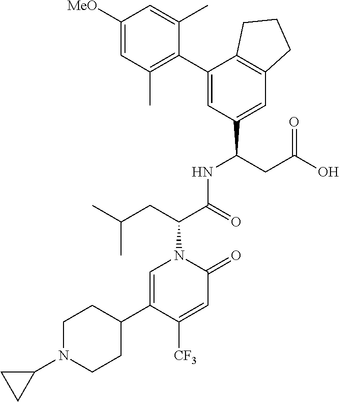 [SMILES: COc1cc(C)c(-c2cc([C@@H](CC(=O)O)NC(=O)[C@@H](CC(C)C)n3cc(C4CCN(C5CC5)CC4)c(C(F)(F)F)cc3=O)cc3c2CCC3)c(C)c1] | 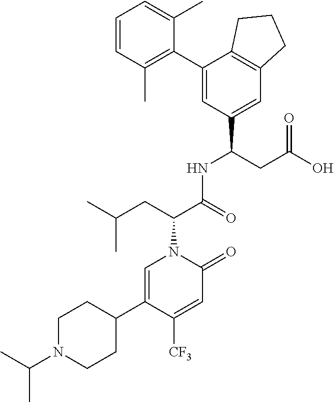 [SMILES: Cc1cccc(C)c1-c1cc([C@@H](CC(=O)O)NC(=O)[C@@H](CC(C)C)n2cc(C3CCN(C(C)C)CC3)c(C(F)(F)F)cc2=O)cc2c1CCC2] |
| 270 | 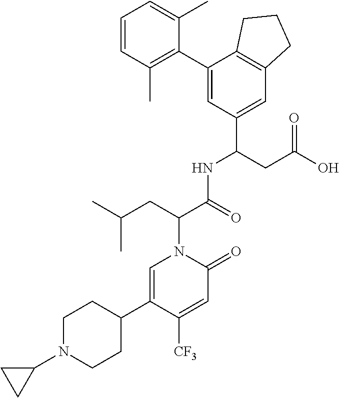 [SMILES: Cc1cccc(C)c1-c1cc(C(CC(=O)O)NC(=O)C(CC(C)C)n2cc(C3CCN(C4CC4)CC3)c(C(F)(F)F)cc2=O)cc2c1CCC2] | 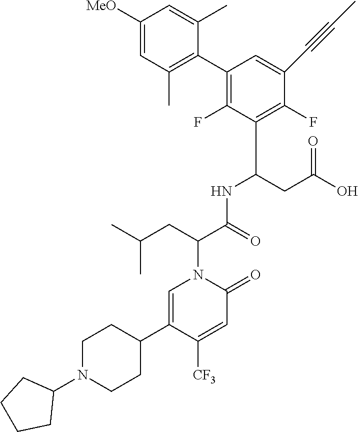 [SMILES: CC#Cc1cc(-c2c(C)cc(OC)cc2C)c(F)c(C(CC(=O)O)NC(=O)C(CC(C)C)n2cc(C3CCN(C4CCCC4)CC3)c(C(F)(F)F)cc2=O)c1F] |
| 271 | 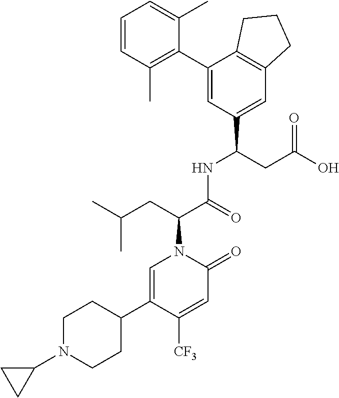 [SMILES: Cc1cccc(C)c1-c1cc([C@@H](CC(=O)O)NC(=O)[C@H](CC(C)C)n2cc(C3CCN(C4CC4)CC3)c(C(F)(F)F)cc2=O)cc2c1CCC2] | 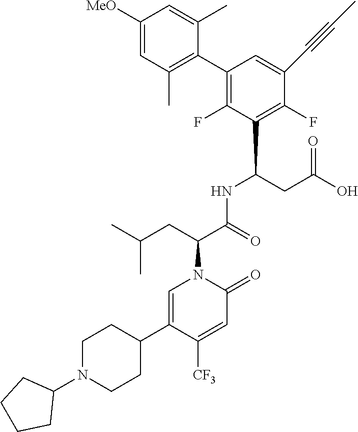 [SMILES: CC#Cc1cc(-c2c(C)cc(OC)cc2C)c(F)c([C@@H](CC(=O)O)NC(=O)[C@H](CC(C)C)n2cc(C3CCN(C4CCCC4)CC3)c(C(F)(F)F)cc2=O)c1F] |
| 272 | 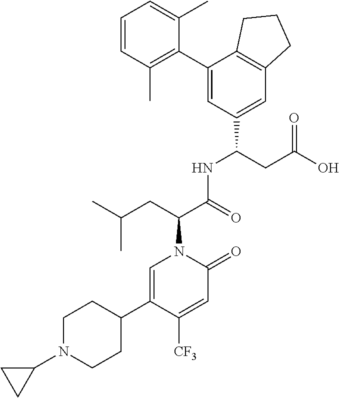 [SMILES: Cc1cccc(C)c1-c1cc([C@H](CC(=O)O)NC(=O)[C@H](CC(C)C)n2cc(C3CCN(C4CC4)CC3)c(C(F)(F)F)cc2=O)cc2c1CCC2] | 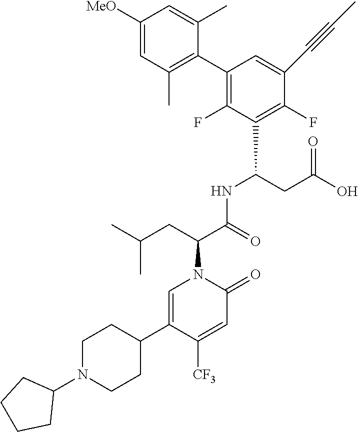 [SMILES: CC#Cc1cc(-c2c(C)cc(OC)cc2C)c(F)c([C@H](CC(=O)O)NC(=O)[C@H](CC(C)C)n2cc(C3CCN(C4CCCC4)CC3)c(C(F)(F)F)cc2=O)c1F] |
| 273 | 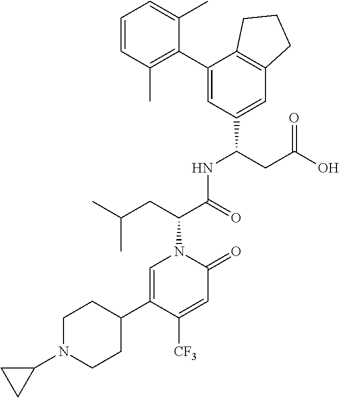 [SMILES: Cc1cccc(C)c1-c1cc([C@H](CC(=O)O)NC(=O)[C@@H](CC(C)C)n2cc(C3CCN(C4CC4)CC3)c(C(F)(F)F)cc2=O)cc2c1CCC2] | 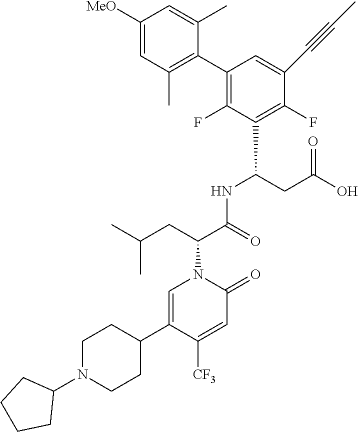 [SMILES: CC#Cc1cc(-c2c(C)cc(OC)cc2C)c(F)c([C@H](CC(=O)O)NC(=O)[C@@H](CC(C)C)n2cc(C3CCN(C4CCCC4)CC3)c(C(F)(F)F)cc2=O)c1F] |
| 274 | 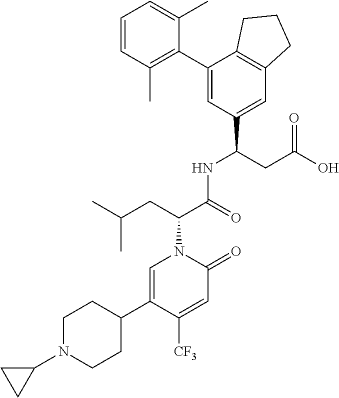 [SMILES: Cc1cccc(C)c1-c1cc([C@@H](CC(=O)O)NC(=O)[C@@H](CC(C)C)n2cc(C3CCN(C4CC4)CC3)c(C(F)(F)F)cc2=O)cc2c1CCC2] | 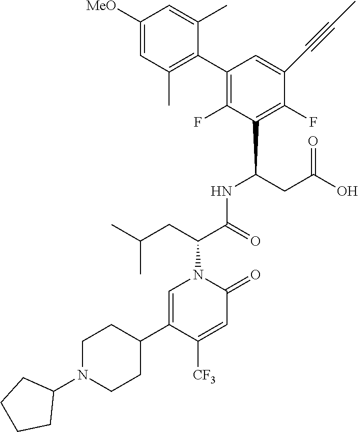 [SMILES: CC#Cc1cc(-c2c(C)cc(OC)cc2C)c(F)c([C@@H](CC(=O)O)NC(=O)[C@@H](CC(C)C)n2cc(C3CCN(C4CCCC4)CC3)c(C(F)(F)F)cc2=O)c1F] |
| 275 | 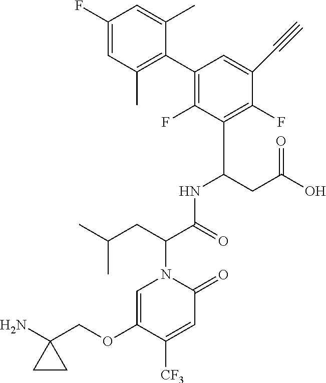 [SMILES: C#Cc1cc(-c2c(C)cc(F)cc2C)c(F)c(C(CC(=O)O)NC(=O)C(CC(C)C)n2cc(OCC3(N)CC3)c(C(F)(F)F)cc2=O)c1F] | 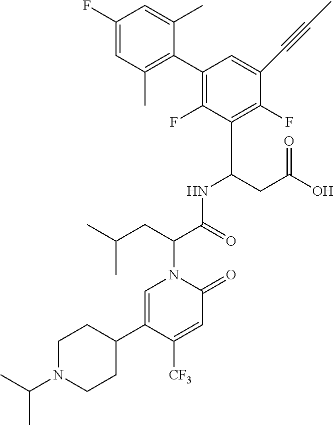 [SMILES: CC#Cc1cc(-c2c(C)cc(F)cc2C)c(F)c(C(CC(=O)O)NC(=O)C(CC(C)C)n2cc(C3CCN(C(C)C)CC3)c(C(F)(F)F)cc2=O)c1F] |
| 276 | 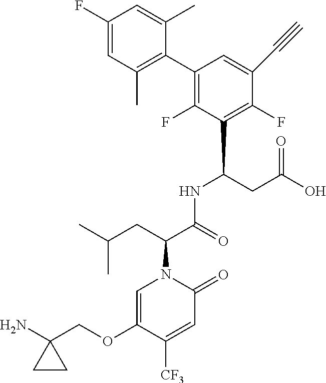 [SMILES: C#Cc1cc(-c2c(C)cc(F)cc2C)c(F)c([C@@H](CC(=O)O)NC(=O)[C@H](CC(C)C)n2cc(OCC3(N)CC3)c(C(F)(F)F)cc2=O)c1F] | 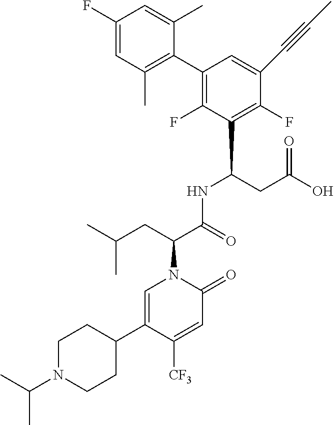 [SMILES: CC#Cc1cc(-c2c(C)cc(F)cc2C)c(F)c([C@@H](CC(=O)O)NC(=O)[C@H](CC(C)C)n2cc(C3CCN(C(C)C)CC3)c(C(F)(F)F)cc2=O)c1F] |
| 277 | 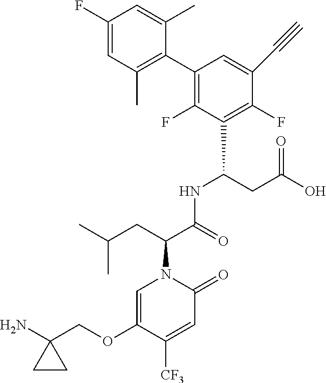 [SMILES: C#Cc1cc(-c2c(C)cc(F)cc2C)c(F)c([C@H](CC(=O)O)NC(=O)[C@H](CC(C)C)n2cc(OCC3(N)CC3)c(C(F)(F)F)cc2=O)c1F] | 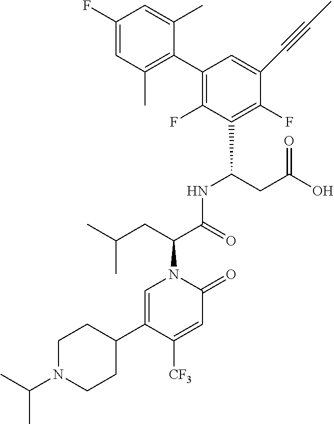 [SMILES: CC#Cc1cc(-c2c(C)cc(F)cc2C)c(F)c([C@H](CC(=O)O)NC(=O)[C@H](CC(C)C)n2cc(C3CCN(C(C)C)CC3)c(C(F)(F)F)cc2=O)c1F] |
| 278 | 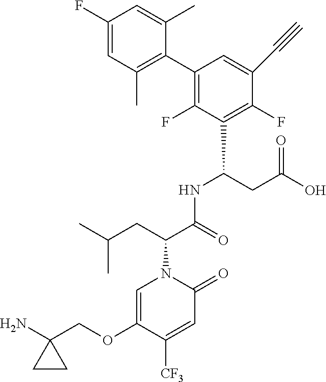 [SMILES: C#Cc1cc(-c2c(C)cc(F)cc2C)c(F)c([C@H](CC(=O)O)NC(=O)[C@@H](CC(C)C)n2cc(OCC3(N)CC3)c(C(F)(F)F)cc2=O)c1F] | 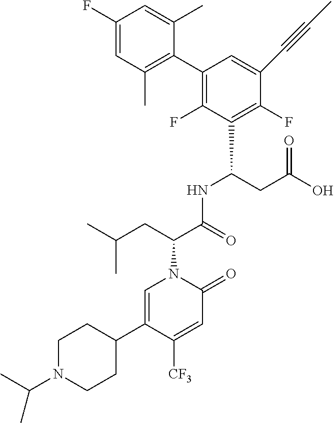 [SMILES: CC#Cc1cc(-c2c(C)cc(F)cc2C)c(F)c([C@H](CC(=O)O)NC(=O)[C@@H](CC(C)C)n2cc(C3CCN(C(C)C)CC3)c(C(F)(F)F)cc2=O)c1F] |
| 279 | 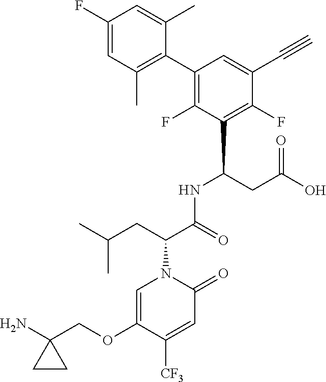 [SMILES: C#Cc1cc(-c2c(C)cc(F)cc2C)c(F)c([C@@H](CC(=O)O)NC(=O)[C@@H](CC(C)C)n2cc(OCC3(N)CC3)c(C(F)(F)F)cc2=O)c1F] | 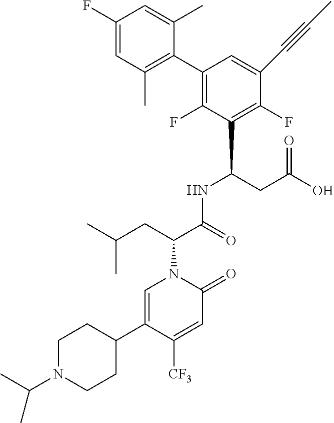 [SMILES: CC#Cc1cc(-c2c(C)cc(F)cc2C)c(F)c([C@@H](CC(=O)O)NC(=O)[C@@H](CC(C)C)n2cc(C3CCN(C(C)C)CC3)c(C(F)(F)F)cc2=O)c1F] |
| 280 | 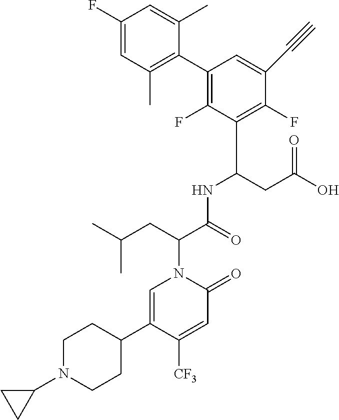 [SMILES: C#Cc1cc(-c2c(C)cc(F)cc2C)c(F)c(C(CC(=O)O)NC(=O)C(CC(C)C)n2cc(C3CCN(C4CC4)CC3)c(C(F)(F)F)cc2=O)c1F] | 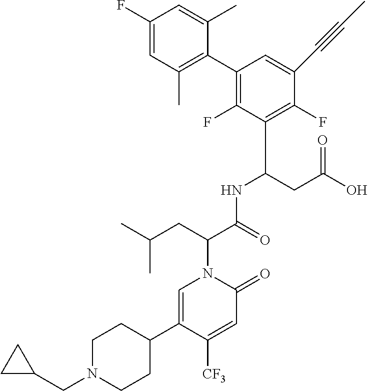 [SMILES: CC#Cc1cc(-c2c(C)cc(F)cc2C)c(F)c(C(CC(=O)O)NC(=O)C(CC(C)C)n2cc(C3CCN(CC4CC4)CC3)c(C(F)(F)F)cc2=O)c1F] |
| 281 | 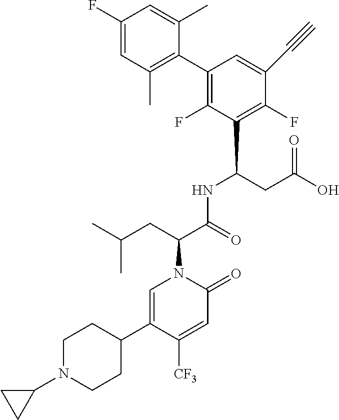 [SMILES: C#Cc1cc(-c2c(C)cc(F)cc2C)c(F)c([C@@H](CC(=O)O)NC(=O)[C@H](CC(C)C)n2cc(C3CCN(C4CC4)CC3)c(C(F)(F)F)cc2=O)c1F] | 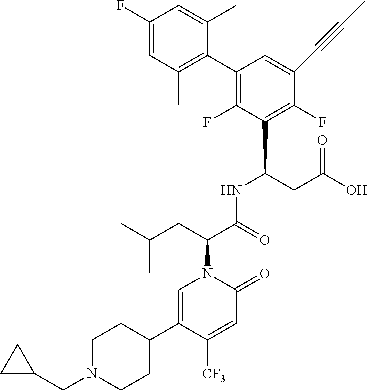 [SMILES: CC#Cc1cc(-c2c(C)cc(F)cc2C)c(F)c([C@@H](CC(=O)O)NC(=O)[C@H](CC(C)C)n2cc(C3CCN(CC4CC4)CC3)c(C(F)(F)F)cc2=O)c1F] |
| 282 | 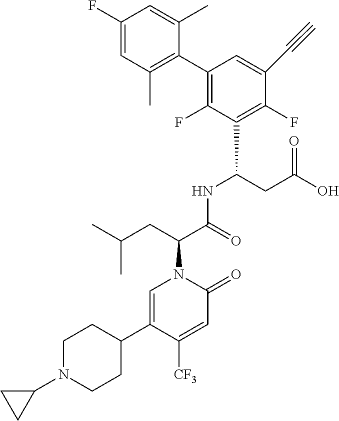 [SMILES: C#Cc1cc(-c2c(C)cc(F)cc2C)c(F)c([C@H](CC(=O)O)NC(=O)[C@H](CC(C)C)n2cc(C3CCN(C4CC4)CC3)c(C(F)(F)F)cc2=O)c1F] | 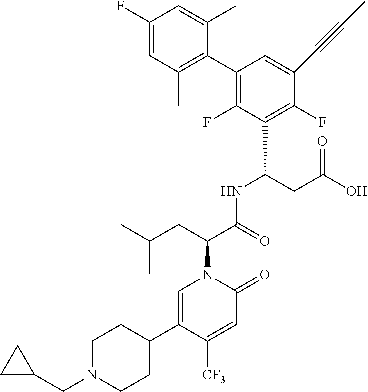 [SMILES: CC#Cc1cc(-c2c(C)cc(F)cc2C)c(F)c([C@H](CC(=O)O)NC(=O)[C@H](CC(C)C)n2cc(C3CCN(CC4CC4)CC3)c(C(F)(F)F)cc2=O)c1F] |
| 283 | 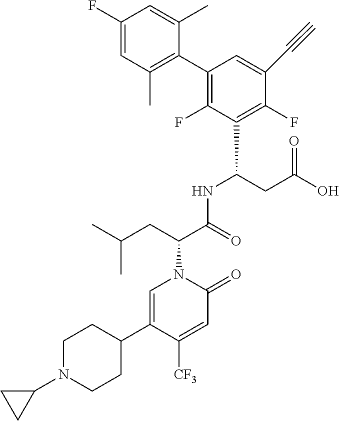 [SMILES: C#Cc1cc(-c2c(C)cc(F)cc2C)c(F)c([C@H](CC(=O)O)NC(=O)[C@@H](CC(C)C)n2cc(C3CCN(C4CC4)CC3)c(C(F)(F)F)cc2=O)c1F] | 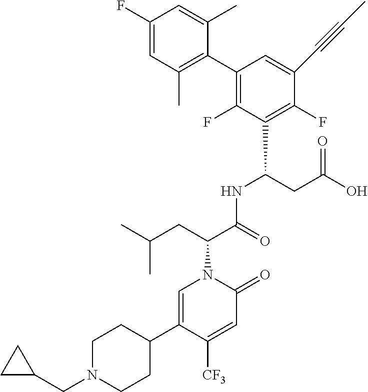 [SMILES: CC#Cc1cc(-c2c(C)cc(F)cc2C)c(F)c([C@H](CC(=O)O)NC(=O)[C@@H](CC(C)C)n2cc(C3CCN(CC4CC4)CC3)c(C(F)(F)F)cc2=O)c1F] |
| 284 | 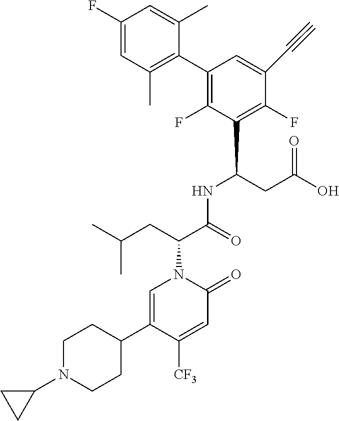 [SMILES: C#Cc1cc(-c2c(C)cc(F)cc2C)c(F)c([C@@H](CC(=O)O)NC(=O)[C@@H](CC(C)C)n2cc(C3CCN(C4CC4)CC3)c(C(F)(F)F)cc2=O)c1F] | 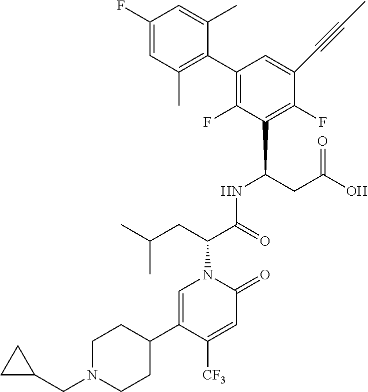 [SMILES: CC#Cc1cc(-c2c(C)cc(F)cc2C)c(F)c([C@@H](CC(=O)O)NC(=O)[C@@H](CC(C)C)n2cc(C3CCN(CC4CC4)CC3)c(C(F)(F)F)cc2=O)c1F] |
| 285 | 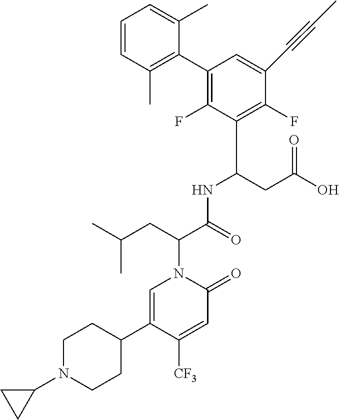 [SMILES: CC#Cc1cc(-c2c(C)cccc2C)c(F)c(C(CC(=O)O)NC(=O)C(CC(C)C)n2cc(C3CCN(C4CC4)CC3)c(C(F)(F)F)cc2=O)c1F] | 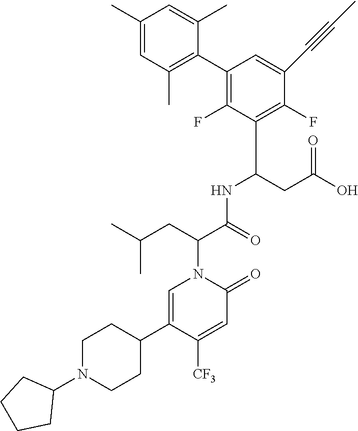 [SMILES: CC#Cc1cc(-c2c(C)cc(C)cc2C)c(F)c(C(CC(=O)O)NC(=O)C(CC(C)C)n2cc(C3CCN(C4CCCC4)CC3)c(C(F)(F)F)cc2=O)c1F] |
| 286 | 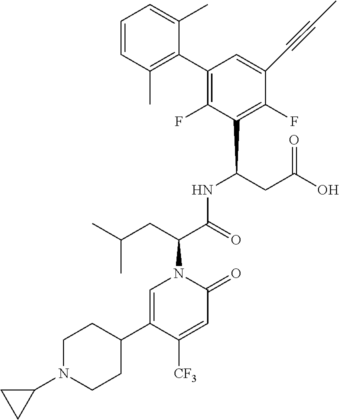 [SMILES: CC#Cc1cc(-c2c(C)cccc2C)c(F)c([C@@H](CC(=O)O)NC(=O)[C@H](CC(C)C)n2cc(C3CCN(C4CC4)CC3)c(C(F)(F)F)cc2=O)c1F] | 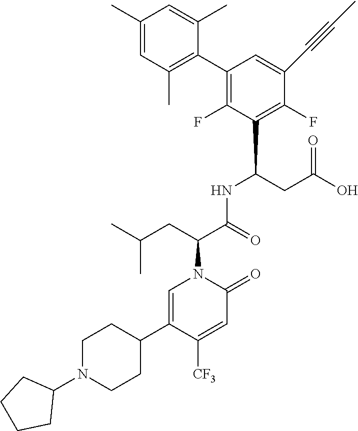 [SMILES: CC#Cc1cc(-c2c(C)cc(C)cc2C)c(F)c([C@@H](CC(=O)O)NC(=O)[C@H](CC(C)C)n2cc(C3CCN(C4CCCC4)CC3)c(C(F)(F)F)cc2=O)c1F] |
| 287 | 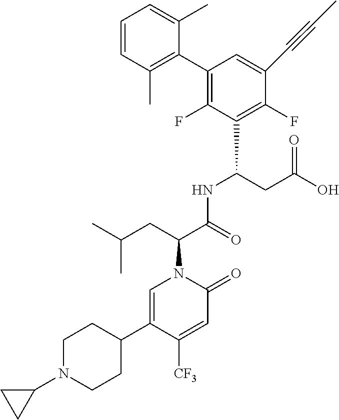 [SMILES: CC#Cc1cc(-c2c(C)cccc2C)c(F)c([C@H](CC(=O)O)NC(=O)[C@H](CC(C)C)n2cc(C3CCN(C4CC4)CC3)c(C(F)(F)F)cc2=O)c1F] | 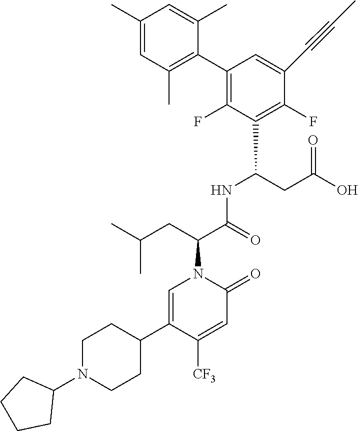 [SMILES: CC#Cc1cc(-c2c(C)cc(C)cc2C)c(F)c([C@H](CC(=O)O)NC(=O)[C@H](CC(C)C)n2cc(C3CCN(C4CCCC4)CC3)c(C(F)(F)F)cc2=O)c1F] |
| 288 | 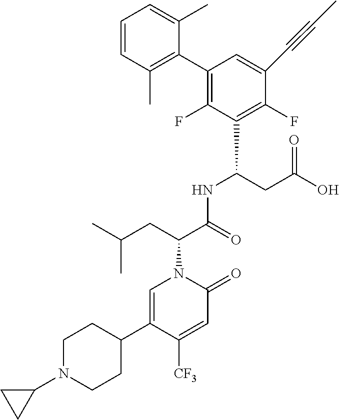 [SMILES: CC#Cc1cc(-c2c(C)cccc2C)c(F)c([C@H](CC(=O)O)NC(=O)[C@@H](CC(C)C)n2cc(C3CCN(C4CC4)CC3)c(C(F)(F)F)cc2=O)c1F] | 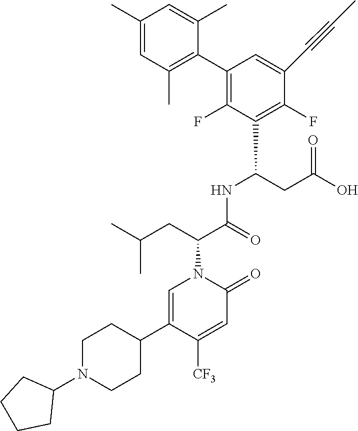 [SMILES: CC#Cc1cc(-c2c(C)cc(C)cc2C)c(F)c([C@H](CC(=O)O)NC(=O)[C@@H](CC(C)C)n2cc(C3CCN(C4CCCC4)CC3)c(C(F)(F)F)cc2=O)c1F] |
| 289 | 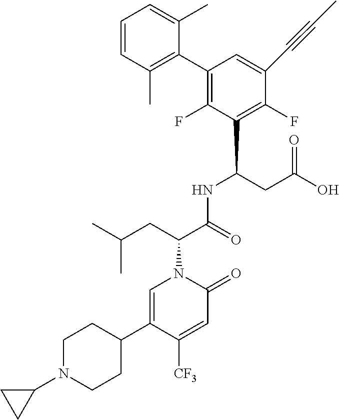 [SMILES: CC#Cc1cc(-c2c(C)cccc2C)c(F)c([C@@H](CC(=O)O)NC(=O)[C@@H](CC(C)C)n2cc(C3CCN(C4CC4)CC3)c(C(F)(F)F)cc2=O)c1F] | 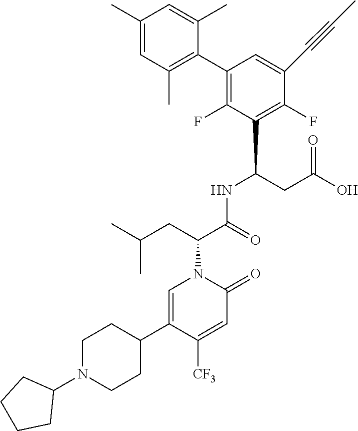 [SMILES: CC#Cc1cc(-c2c(C)cc(C)cc2C)c(F)c([C@@H](CC(=O)O)NC(=O)[C@@H](CC(C)C)n2cc(C3CCN(C4CCCC4)CC3)c(C(F)(F)F)cc2=O)c1F] |
| 290 | 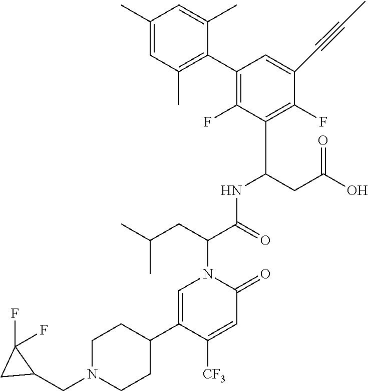 [SMILES: CC#Cc1cc(-c2c(C)cc(C)cc2C)c(F)c(C(CC(=O)O)NC(=O)C(CC(C)C)n2cc(C3CCN(CC4CC4(F)F)CC3)c(C(F)(F)F)cc2=O)c1F] | 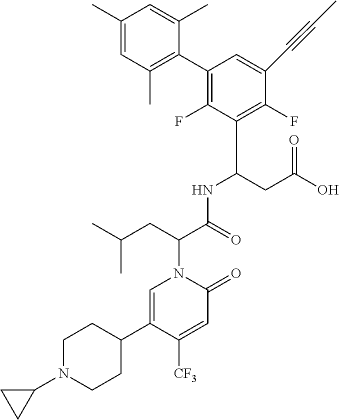 [SMILES: CC#Cc1cc(-c2c(C)cc(C)cc2C)c(F)c(C(CC(=O)O)NC(=O)C(CC(C)C)n2cc(C3CCN(C4CC4)CC3)c(C(F)(F)F)cc2=O)c1F] |
| 291 | 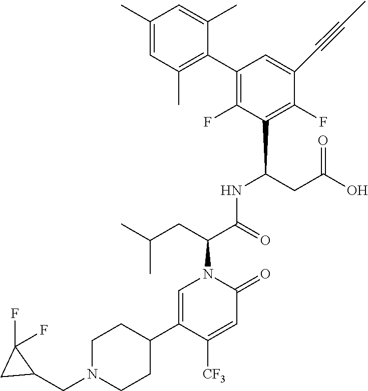 [SMILES: CC#Cc1cc(-c2c(C)cc(C)cc2C)c(F)c([C@@H](CC(=O)O)NC(=O)[C@H](CC(C)C)n2cc(C3CCN(CC4CC4(F)F)CC3)c(C(F)(F)F)cc2=O)c1F] | 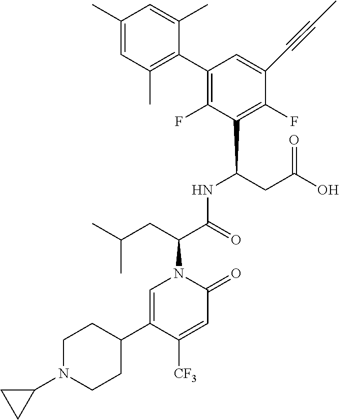 [SMILES: CC#Cc1cc(-c2c(C)cc(C)cc2C)c(F)c([C@@H](CC(=O)O)NC(=O)[C@H](CC(C)C)n2cc(C3CCN(C4CC4)CC3)c(C(F)(F)F)cc2=O)c1F] |
| 292 | 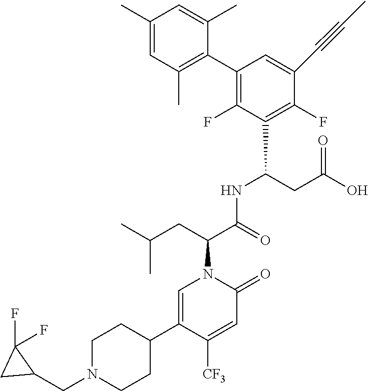 [SMILES: CC#Cc1cc(-c2c(C)cc(C)cc2C)c(F)c([C@H](CC(=O)O)NC(=O)[C@H](CC(C)C)n2cc(C3CCN(CC4CC4(F)F)CC3)c(C(F)(F)F)cc2=O)c1F] | 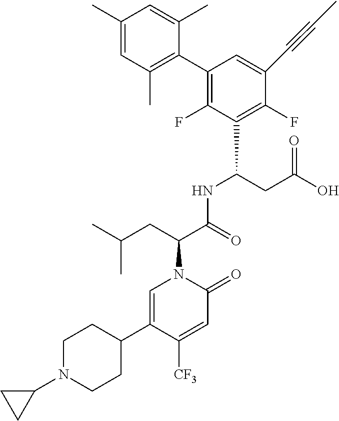 [SMILES: CC#Cc1cc(-c2c(C)cc(C)cc2C)c(F)c([C@H](CC(=O)O)NC(=O)[C@H](CC(C)C)n2cc(C3CCN(C4CC4)CC3)c(C(F)(F)F)cc2=O)c1F] |
| 293 | 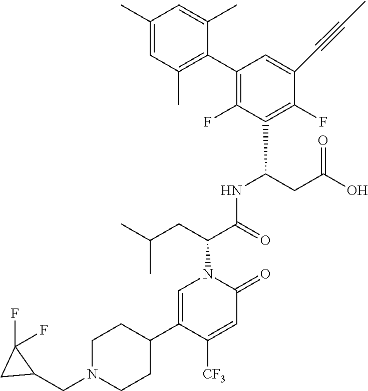 [SMILES: CC#Cc1cc(-c2c(C)cc(C)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] | 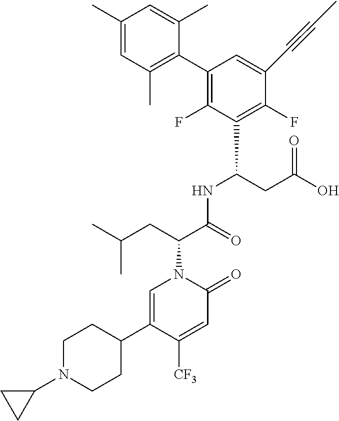 [SMILES: CC#Cc1cc(-c2c(C)cc(C)cc2C)c(F)c([C@H](CC(=O)O)NC(=O)[C@@H](CC(C)C)n2cc(C3CCN(C4CC4)CC3)c(C(F)(F)F)cc2=O)c1F] |
| 294 | 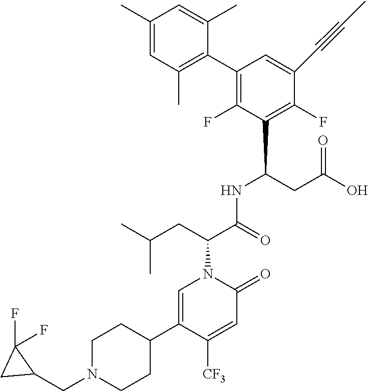 [SMILES: CC#Cc1cc(-c2c(C)cc(C)cc2C)c(F)c([C@@H](CC(=O)O)NC(=O)[C@@H](CC(C)C)n2cc(C3CCN(CC4CC4(F)F)CC3)c(C(F)(F)F)cc2=O)c1F] | 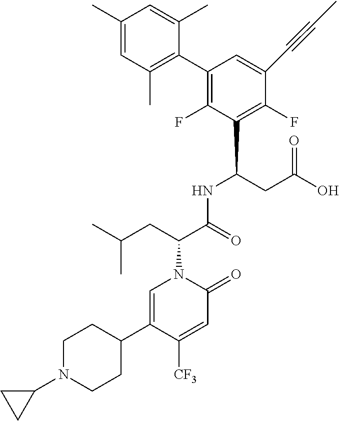 [SMILES: CC#Cc1cc(-c2c(C)cc(C)cc2C)c(F)c([C@@H](CC(=O)O)NC(=O)[C@@H](CC(C)C)n2cc(C3CCN(C4CC4)CC3)c(C(F)(F)F)cc2=O)c1F] |
| 295 | 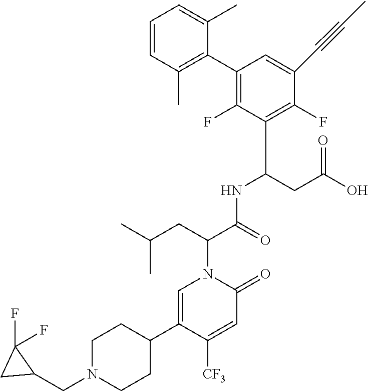 [SMILES: CC#Cc1cc(-c2c(C)cccc2C)c(F)c(C(CC(=O)O)NC(=O)C(CC(C)C)n2cc(C3CCN(CC4CC4(F)F)CC3)c(C(F)(F)F)cc2=O)c1F] | 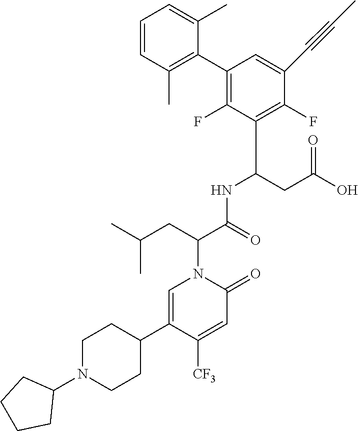 [SMILES: CC#Cc1cc(-c2c(C)cccc2C)c(F)c(C(CC(=O)O)NC(=O)C(CC(C)C)n2cc(C3CCN(C4CCCC4)CC3)c(C(F)(F)F)cc2=O)c1F] |
| 296 | 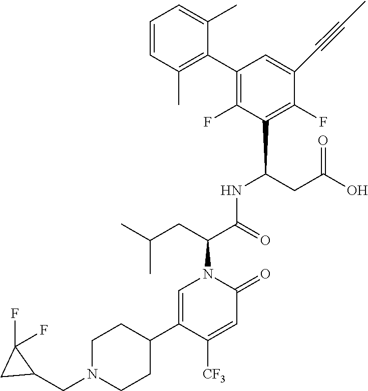 [SMILES: CC#Cc1cc(-c2c(C)cccc2C)c(F)c([C@@H](CC(=O)O)NC(=O)[C@H](CC(C)C)n2cc(C3CCN(CC4CC4(F)F)CC3)c(C(F)(F)F)cc2=O)c1F] | 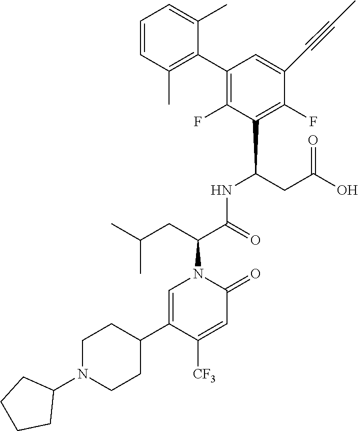 [SMILES: CC#Cc1cc(-c2c(C)cccc2C)c(F)c([C@@H](CC(=O)O)NC(=O)[C@H](CC(C)C)n2cc(C3CCN(C4CCCC4)CC3)c(C(F)(F)F)cc2=O)c1F] |
| 297 | 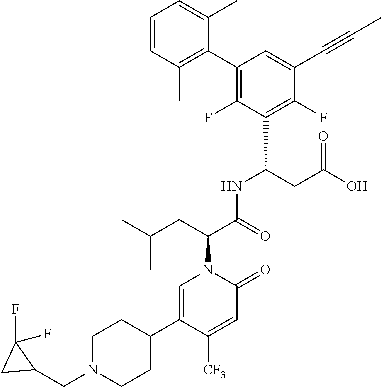 [SMILES: CC#Cc1cc(-c2c(C)cccc2C)c(F)c([C@H](CC(=O)O)NC(=O)[C@H](CC(C)C)n2cc(C3CCN(CC4CC4(F)F)CC3)c(C(F)(F)F)cc2=O)c1F] | 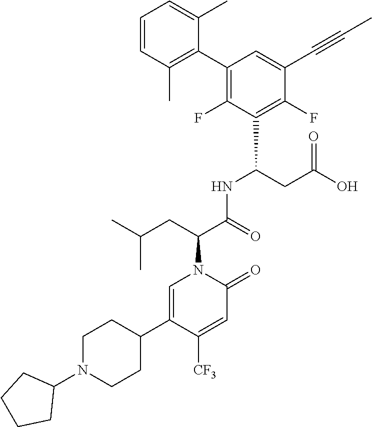 [SMILES: CC#Cc1cc(-c2c(C)cccc2C)c(F)c([C@H](CC(=O)O)NC(=O)[C@H](CC(C)C)n2cc(C3CCN(C4CCCC4)CC3)c(C(F)(F)F)cc2=O)c1F] |
| 298 | 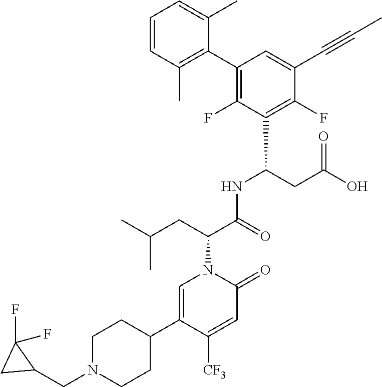 [SMILES: CC#Cc1cc(-c2c(C)cccc2C)c(F)c([C@H](CC(=O)O)NC(=O)[C@@H](CC(C)C)n2cc(C3CCN(CC4CC4(F)F)CC3)c(C(F)(F)F)cc2=O)c1F] | 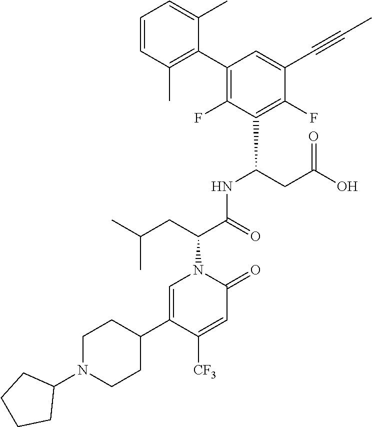 [SMILES: CC#Cc1cc(-c2c(C)cccc2C)c(F)c([C@H](CC(=O)O)NC(=O)[C@@H](CC(C)C)n2cc(C3CCN(C4CCCC4)CC3)c(C(F)(F)F)cc2=O)c1F] |
| 299 | 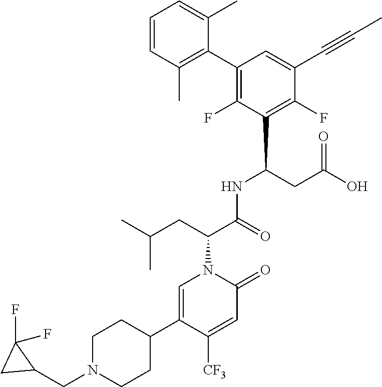 [SMILES: CC#Cc1cc(-c2c(C)cccc2C)c(F)c([C@@H](CC(=O)O)NC(=O)[C@@H](CC(C)C)n2cc(C3CCN(CC4CC4(F)F)CC3)c(C(F)(F)F)cc2=O)c1F] | 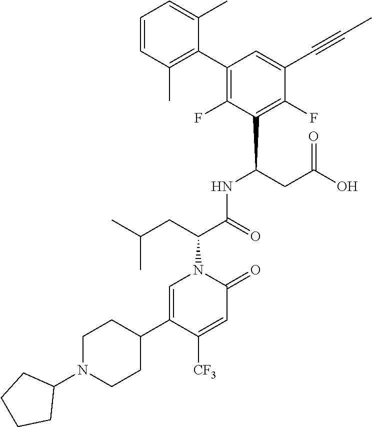 [SMILES: CC#Cc1cc(-c2c(C)cccc2C)c(F)c([C@@H](CC(=O)O)NC(=O)[C@@H](CC(C)C)n2cc(C3CCN(C4CCCC4)CC3)c(C(F)(F)F)cc2=O)c1F] |
| 300 | 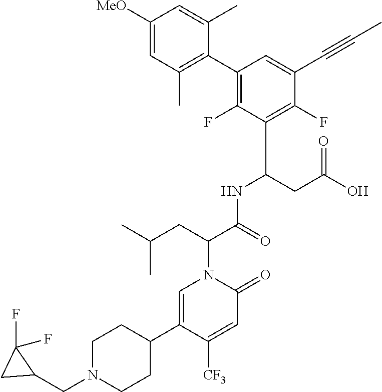 [SMILES: CC#Cc1cc(-c2c(C)cc(OC)cc2C)c(F)c(C(CC(=O)O)NC(=O)C(CC(C)C)n2cc(C3CCN(CC4CC4(F)F)CC3)c(C(F)(F)F)cc2=O)c1F] | 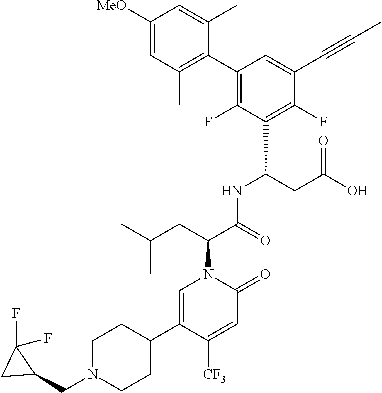 [SMILES: CC#Cc1cc(-c2c(C)cc(OC)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 301 | 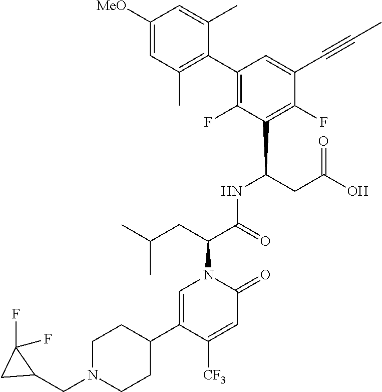 [SMILES: CC#Cc1cc(-c2c(C)cc(OC)cc2C)c(F)c([C@@H](CC(=O)O)NC(=O)[C@H](CC(C)C)n2cc(C3CCN(CC4CC4(F)F)CC3)c(C(F)(F)F)cc2=O)c1F] | 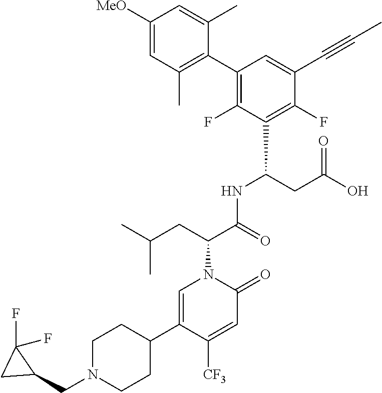 [SMILES: CC#Cc1cc(-c2c(C)cc(OC)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] |
| 302 | 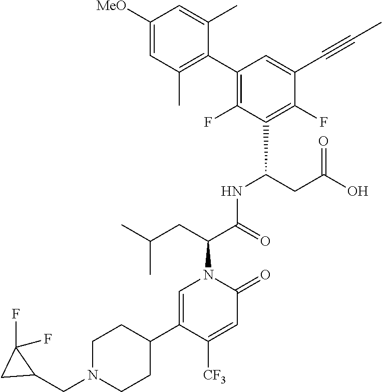 [SMILES: CC#Cc1cc(-c2c(C)cc(OC)cc2C)c(F)c([C@H](CC(=O)O)NC(=O)[C@H](CC(C)C)n2cc(C3CCN(CC4CC4(F)F)CC3)c(C(F)(F)F)cc2=O)c1F] | 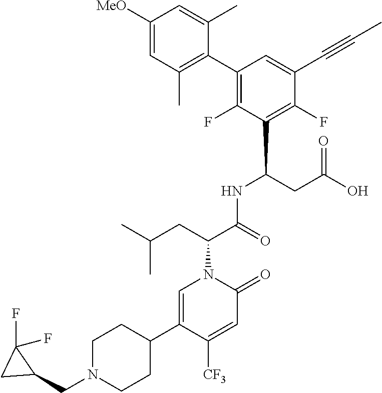 [SMILES: CC#Cc1cc(-c2c(C)cc(OC)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] |
| 303 | 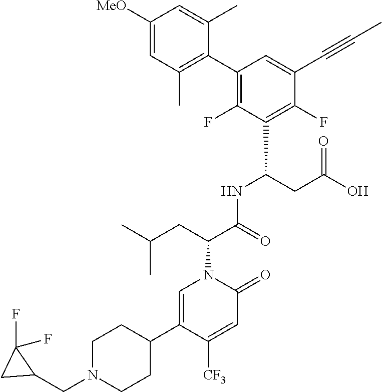 [SMILES: CC#Cc1cc(-c2c(C)cc(OC)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] | 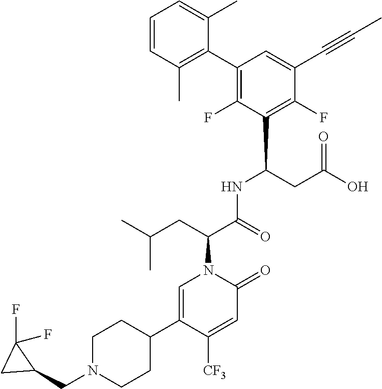 [SMILES: CC#Cc1cc(-c2c(C)cccc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] |
| 304 | 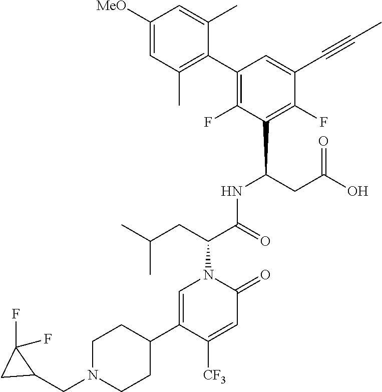 [SMILES: CC#Cc1cc(-c2c(C)cc(OC)cc2C)c(F)c([C@@H](CC(=O)O)NC(=O)[C@@H](CC(C)C)n2cc(C3CCN(CC4CC4(F)F)CC3)c(C(F)(F)F)cc2=O)c1F] | 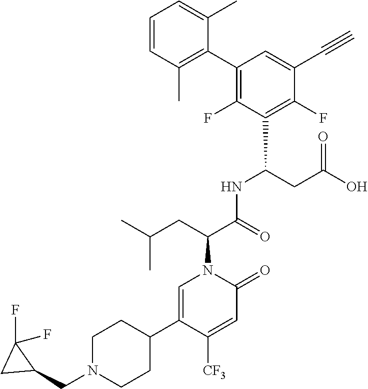 [SMILES: C#Cc1cc(-c2c(C)cccc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 305 | 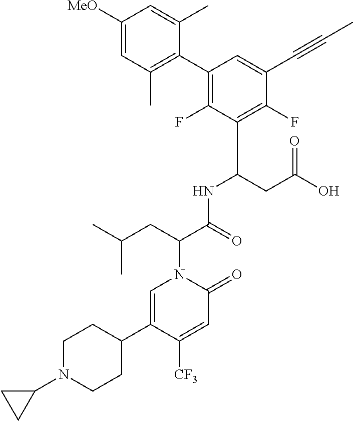 [SMILES: CC#Cc1cc(-c2c(C)cc(OC)cc2C)c(F)c(C(CC(=O)O)NC(=O)C(CC(C)C)n2cc(C3CCN(C4CC4)CC3)c(C(F)(F)F)cc2=O)c1F] | 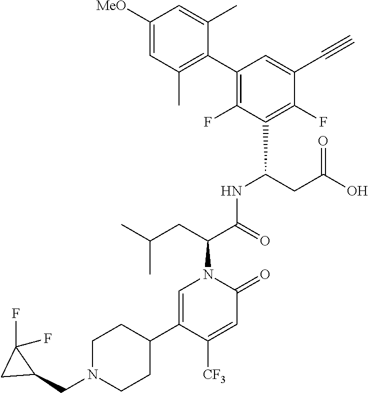 [SMILES: C#Cc1cc(-c2c(C)cc(OC)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 306 | 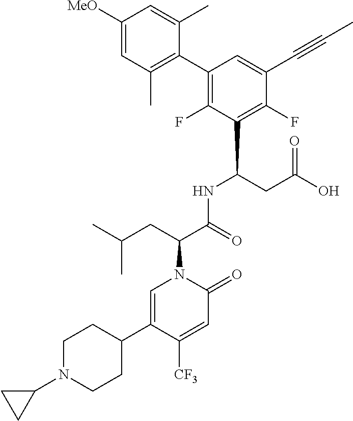 [SMILES: CC#Cc1cc(-c2c(C)cc(OC)cc2C)c(F)c([C@@H](CC(=O)O)NC(=O)[C@H](CC(C)C)n2cc(C3CCN(C4CC4)CC3)c(C(F)(F)F)cc2=O)c1F] | 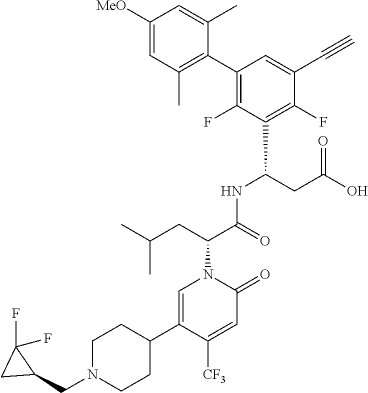 [SMILES: C#Cc1cc(-c2c(C)cc(OC)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] |
| 307 | 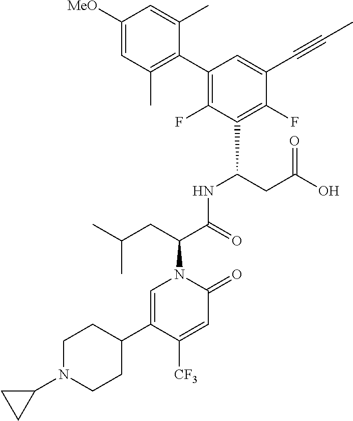 [SMILES: CC#Cc1cc(-c2c(C)cc(OC)cc2C)c(F)c([C@H](CC(=O)O)NC(=O)[C@H](CC(C)C)n2cc(C3CCN(C4CC4)CC3)c(C(F)(F)F)cc2=O)c1F] | 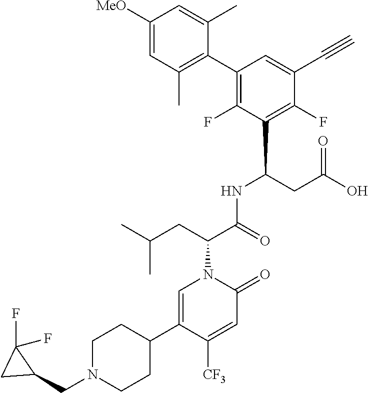 [SMILES: C#Cc1cc(-c2c(C)cc(OC)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] |
| 308 | 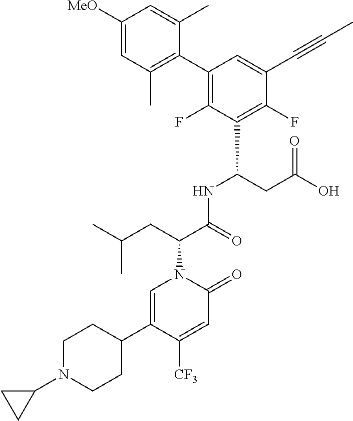 [SMILES: CC#Cc1cc(-c2c(C)cc(OC)cc2C)c(F)c([C@H](CC(=O)O)NC(=O)[C@@H](CC(C)C)n2cc(C3CCN(C4CC4)CC3)c(C(F)(F)F)cc2=O)c1F] | 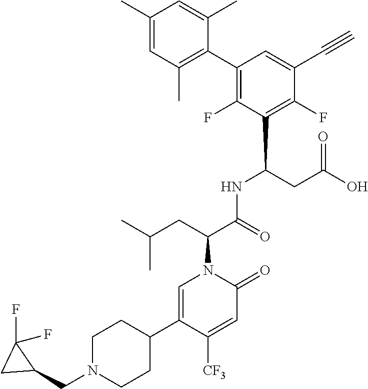 [SMILES: C#Cc1cc(-c2c(C)cc(C)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] |
| 309 | 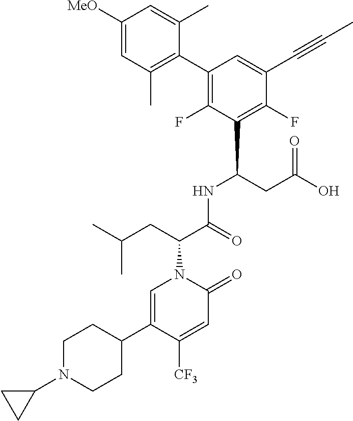 [SMILES: CC#Cc1cc(-c2c(C)cc(OC)cc2C)c(F)c([C@@H](CC(=O)O)NC(=O)[C@@H](CC(C)C)n2cc(C3CCN(C4CC4)CC3)c(C(F)(F)F)cc2=O)c1F] | 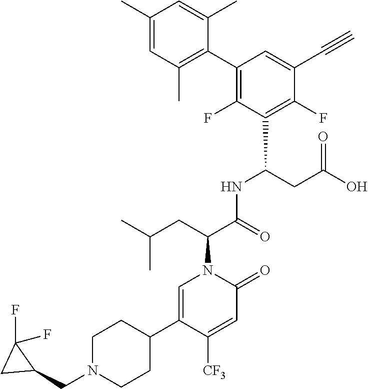 [SMILES: C#Cc1cc(-c2c(C)cc(C)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 310 | 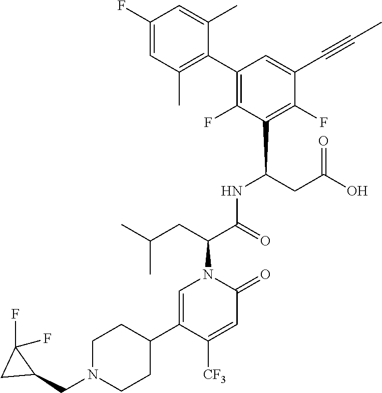 [SMILES: CC#Cc1cc(-c2c(C)cc(F)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] | 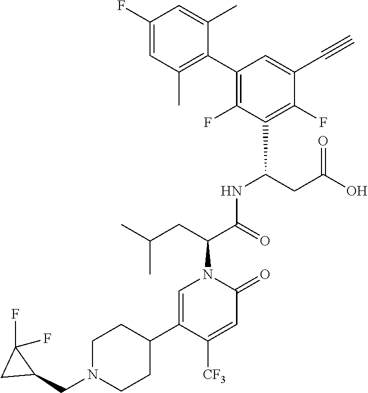 [SMILES: C#Cc1cc(-c2c(C)cc(F)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] |
| 311 | 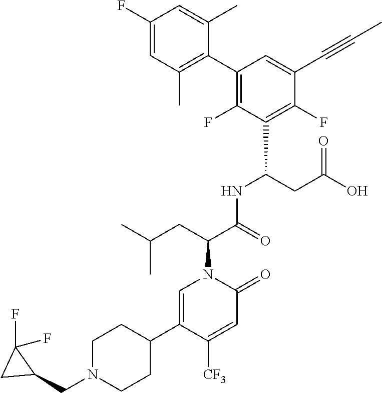 [SMILES: CC#Cc1cc(-c2c(C)cc(F)cc2C)c(F)c([C@H](CC(=O)O)NC(=O)[C@H](CC(C)C)n2cc(C3CCN(C[C@H]4CC4(F)F)CC3)c(C(F)(F)F)cc2=O)c1F] | 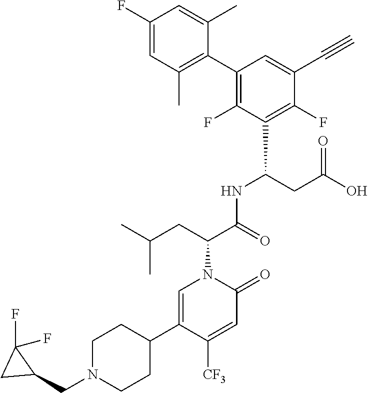 [SMILES: C#Cc1cc(-c2c(C)cc(F)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] |
| 312 | 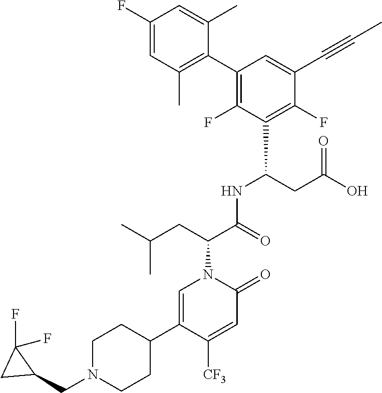 [SMILES: CC#Cc1cc(-c2c(C)cc(F)cc2C)c(F)c([C@H](CC(=O)O)NC(=O)[C@@H](CC(C)C)n2cc(C3CCN(C[C@H]4CC4(F)F)CC3)c(C(F)(F)F)cc2=O)c1F] | 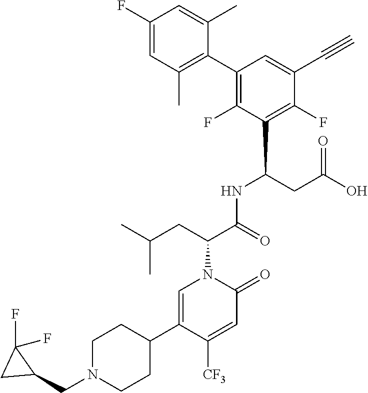 [SMILES: C#Cc1cc(-c2c(C)cc(F)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] |
| 313 | 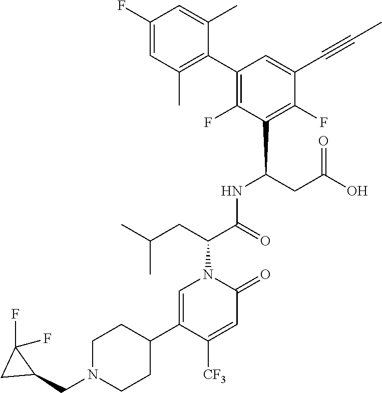 [SMILES: CC#Cc1cc(-c2c(C)cc(F)cc2C)c(F)c([C@@H](CC(=O)O)NC(=O)[C@@H](CC(C)C)n2cc(C3CCN(C[C@H]4CC4(F)F)CC3)c(C(F)(F)F)cc2=O)c1F] | 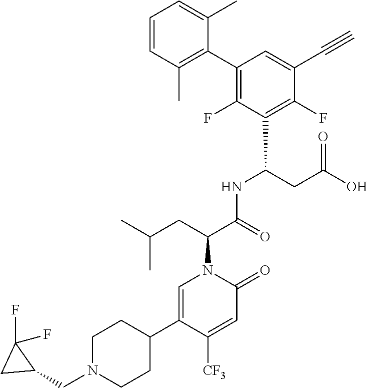 [SMILES: C#Cc1cc(-c2c(C)cccc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 314 | 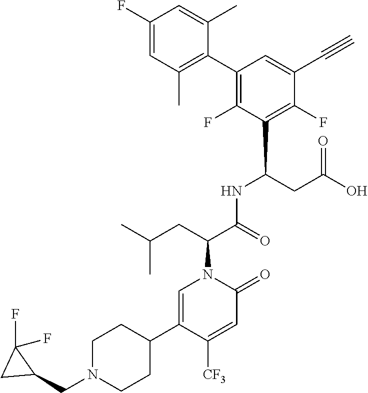 [SMILES: C#Cc1cc(-c2c(C)cc(F)cc2C)c(F)c([C@@H](CC(=O)O)NC(=O)[C@H](CC(C)C)n2cc(C3CCN(C[C@H]4CC4(F)F)CC3)c(C(F)(F)F)cc2=O)c1F] | 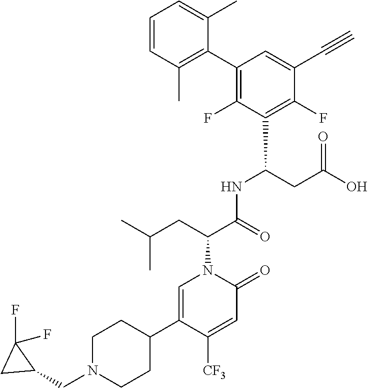 [SMILES: C#Cc1cc(-c2c(C)cccc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] |
| 315 | 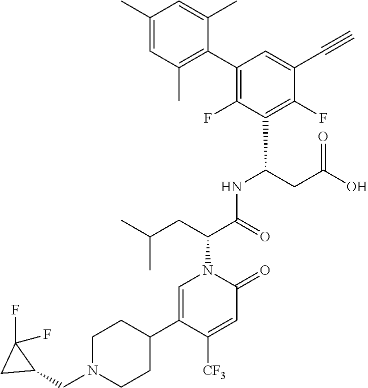 [SMILES: C#Cc1cc(-c2c(C)cc(C)cc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] | 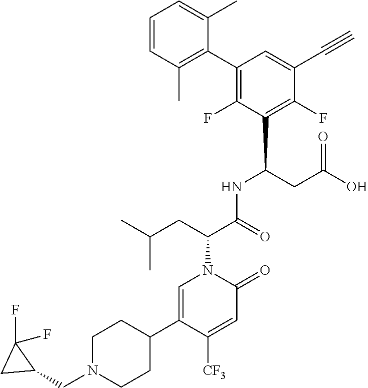 [SMILES: C#Cc1cc(-c2c(C)cccc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] |
| 316 | 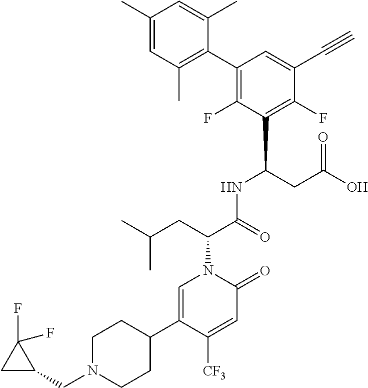 [SMILES: C#Cc1cc(-c2c(C)cc(C)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] | 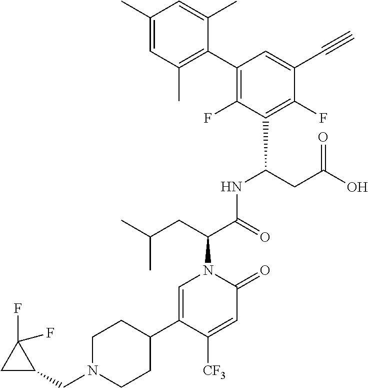 [SMILES: C#Cc1cc(-c2c(C)cc(C)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 317 | 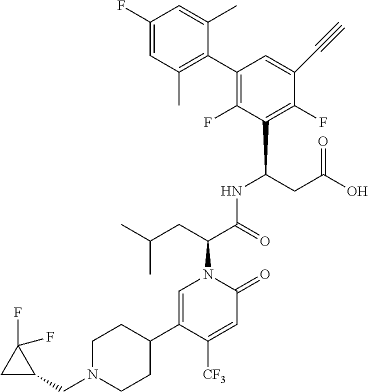 [SMILES: C#Cc1cc(-c2c(C)cc(F)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] | |
| 318 | 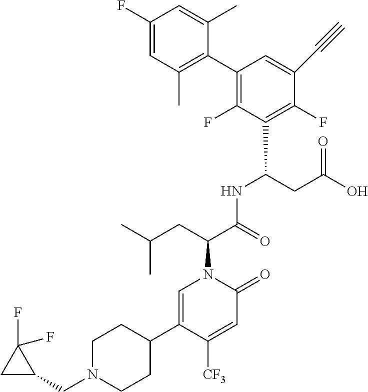 [SMILES: C#Cc1cc(-c2c(C)cc(F)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] | |
| 319 | 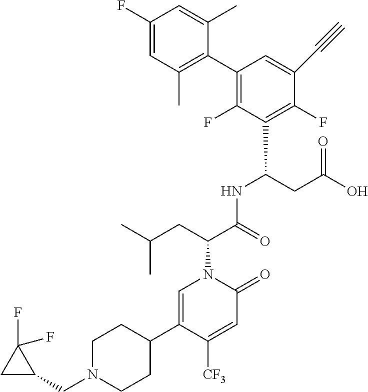 [SMILES: C#Cc1cc(-c2c(C)cc(F)cc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] | 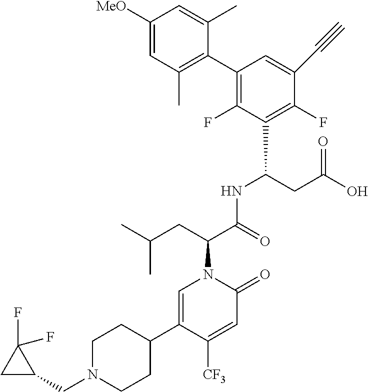 [SMILES: C#Cc1cc(-c2c(C)cc(OC)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 320 | 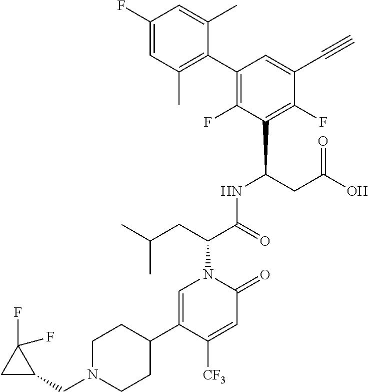 [SMILES: C#Cc1cc(-c2c(C)cc(F)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] | 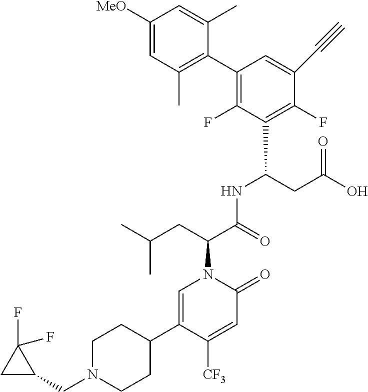 [SMILES: C#Cc1cc(-c2c(C)cc(OC)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 321 | 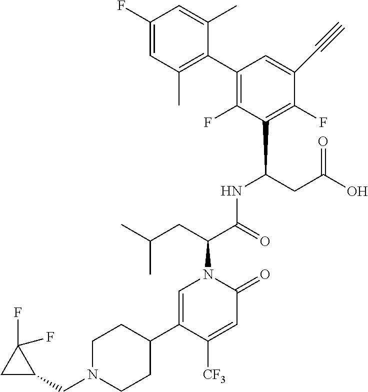 [SMILES: C#Cc1cc(-c2c(C)cc(F)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] | 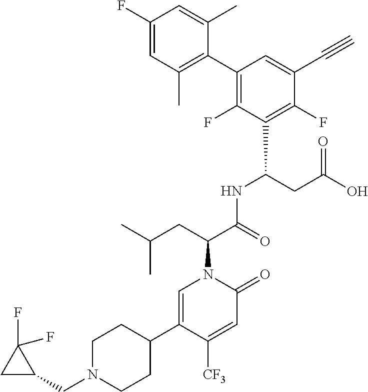 [SMILES: C#Cc1cc(-c2c(C)cc(F)cc2C)c(F)c([C@H](CC(=O)O)NC(=O)[C@H](CC(C)C)n2cc(C3CCN(C[C@@H]4CC4(F)F)CC3)c(C(F)(F)F)cc2=O)c1F] |
| 322 | 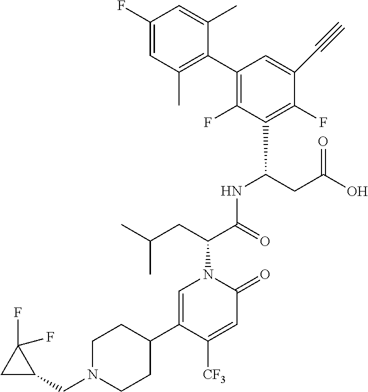 [SMILES: C#Cc1cc(-c2c(C)cc(F)cc2C)c(F)c([C@H](CC(=O)O)NC(=O)[C@@H](CC(C)C)n2cc(C3CCN(C[C@@H]4CC4(F)F)CC3)c(C(F)(F)F)cc2=O)c1F] | 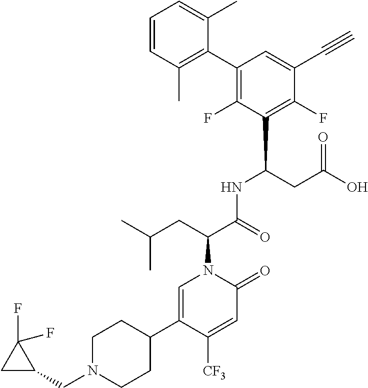 [SMILES: C#Cc1cc(-c2c(C)cccc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] |
| 323 | 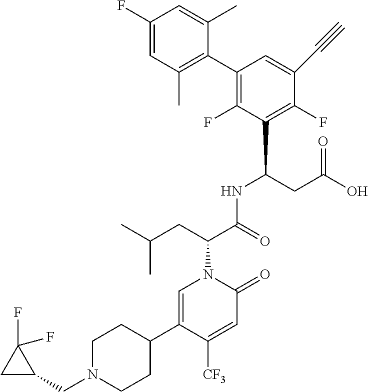 [SMILES: C#Cc1cc(-c2c(C)cc(F)cc2C)c(F)c([C@@H](CC(=O)O)NC(=O)[C@@H](CC(C)C)n2cc(C3CCN(C[C@@H]4CC4(F)F)CC3)c(C(F)(F)F)cc2=O)c1F] | 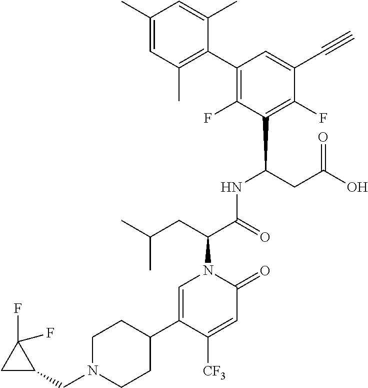 [SMILES: C#Cc1cc(-c2c(C)cc(C)cc2C)c(F)c([C@@H](CC(=O)O)NC(=O)[C@H](CC(C)C)n2cc(C3CCN(C[C@@H]4CC4(F)F)CC3)c(C(F)(F)F)cc2=O)c1F] |
| 324 | 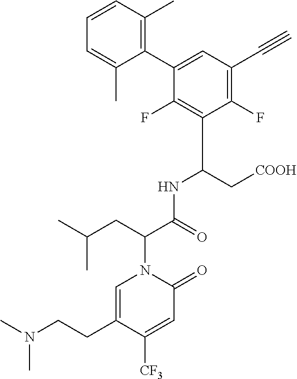 [SMILES: C#Cc1cc(-c2c(C)cccc2C)c(F)c(C(CC(=O)O)NC(=O)C(CC(C)C)n2cc(CCN(C)C)c(C(F)(F)F)cc2=O)c1F] | 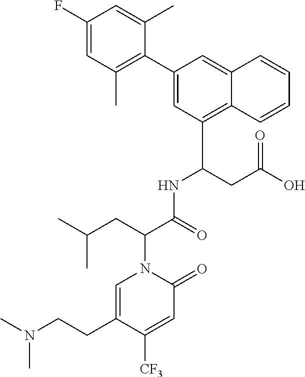 [SMILES: Cc1cc(F)cc(C)c1-c1cc(C(CC(=O)O)NC(=O)C(CC(C)C)n2cc(CCN(C)C)c(C(F)(F)F)cc2=O)c2ccccc2c1] |
| 325 | 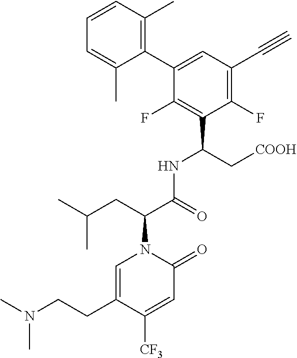 [SMILES: C#Cc1cc(-c2c(C)cccc2C)c(F)c([C@@H](CC(=O)O)NC(=O)[C@H](CC(C)C)n2cc(CCN(C)C)c(C(F)(F)F)cc2=O)c1F] | 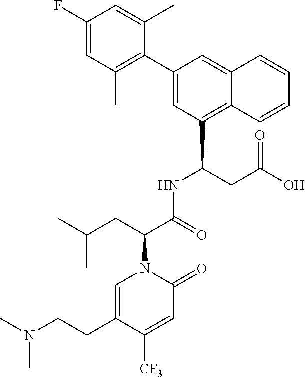 [SMILES: Cc1cc(F)cc(C)c1-c1cc([C@@H](CC(=O)O)NC(=O)[C@H](CC(C)C)n2cc(CCN(C)C)c(C(F)(F)F)cc2=O)c2ccccc2c1] |
| 326 | 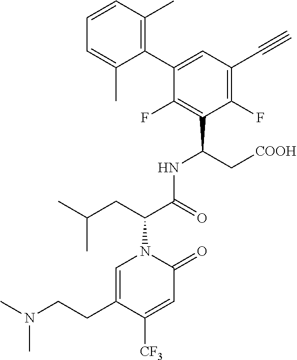 [SMILES: C#Cc1cc(-c2c(C)cccc2C)c(F)c([C@@H](CC(=O)O)NC(=O)[C@@H](CC(C)C)n2cc(CCN(C)C)c(C(F)(F)F)cc2=O)c1F] | 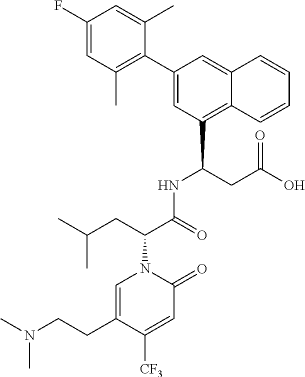 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)c2ccccc2c1] |
| 327 | 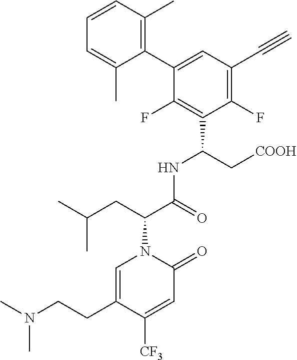 [SMILES: C#Cc1cc(-c2c(C)cccc2C)c(F)c([C@H](CC(=O)O)NC(=O)[C@@H](CC(C)C)n2cc(CCN(C)C)c(C(F)(F)F)cc2=O)c1F] | 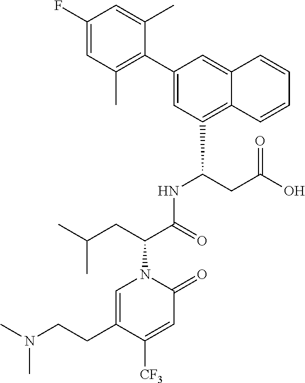 [SMILES: Cc1cc(F)cc(C)c1-c1cc([C@H](CC(=O)O)NC(=O)[C@@H](CC(C)C)n2cc(CCN(C)C)c(C(F)(F)F)cc2=O)c2ccccc2c1] |
| 328 | 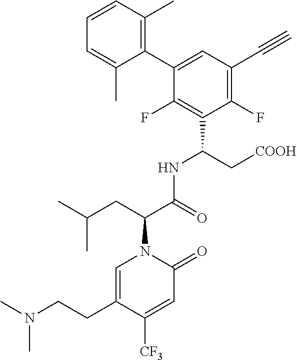 [SMILES: C#Cc1cc(-c2c(C)cccc2C)c(F)c([C@H](CC(=O)O)NC(=O)[C@H](CC(C)C)n2cc(CCN(C)C)c(C(F)(F)F)cc2=O)c1F] | 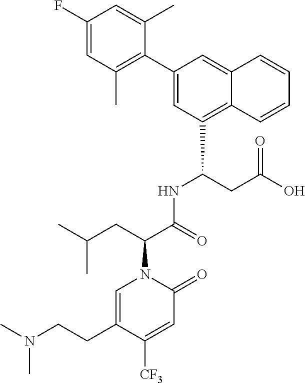 [SMILES: Cc1cc(F)cc(C)c1-c1cc([C@H](CC(=O)O)NC(=O)[C@H](CC(C)C)n2cc(CCN(C)C)c(C(F)(F)F)cc2=O)c2ccccc2c1] |
| 329 | 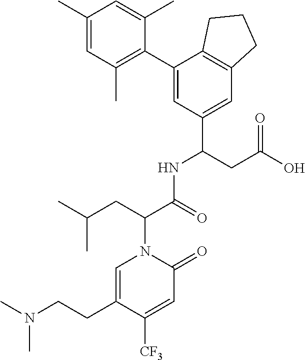 [SMILES: Cc1cc(C)c(-c2cc(C(CC(=O)O)NC(=O)C(CC(C)C)n3cc(CCN(C)C)c(C(F)(F)F)cc3=O)cc3c2CCC3)c(C)c1] | 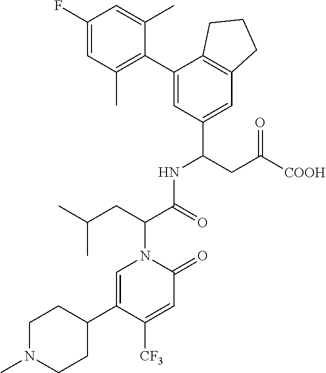 [SMILES: Cc1cc(F)cc(C)c1-c1cc(C(CC(=O)C(=O)O)NC(=O)C(CC(C)C)n2cc(C3CCN(C)CC3)c(C(F)(F)F)cc2=O)cc2c1CCC2] |
| 330 | 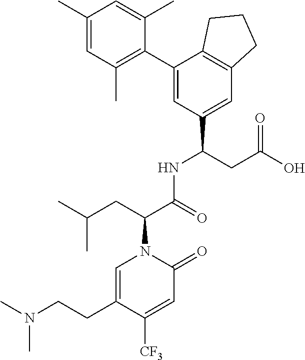 [SMILES: Cc1cc(C)c(-c2cc([C@@H](CC(=O)O)NC(=O)[C@H](CC(C)C)n3cc(CCN(C)C)c(C(F)(F)F)cc3=O)cc3c2CCC3)c(C)c1] | 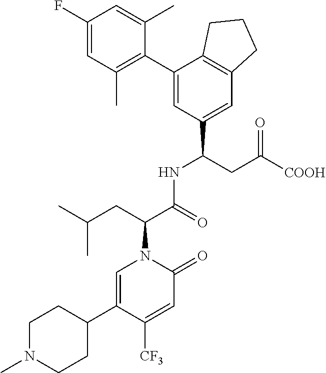 [SMILES: Cc1cc(F)cc(C)c1-c1cc([C@@H](CC(=O)C(=O)O)NC(=O)[C@H](CC(C)C)n2cc(C3CCN(C)CC3)c(C(F)(F)F)cc2=O)cc2c1CCC2] |
| 331 | 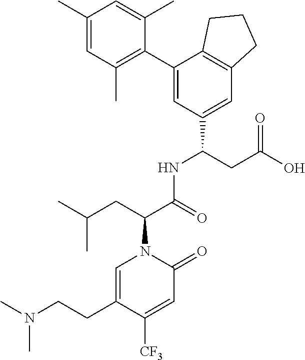 [SMILES: Cc1cc(C)c(-c2cc([C@H](CC(=O)O)NC(=O)[C@H](CC(C)C)n3cc(CCN(C)C)c(C(F)(F)F)cc3=O)cc3c2CCC3)c(C)c1] | 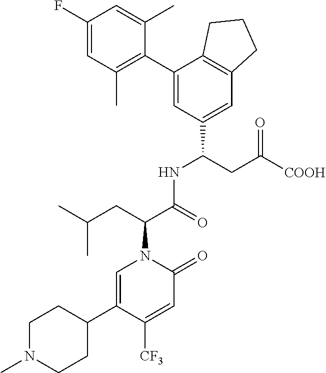 [SMILES: Cc1cc(F)cc(C)c1-c1cc([C@H](CC(=O)C(=O)O)NC(=O)[C@H](CC(C)C)n2cc(C3CCN(C)CC3)c(C(F)(F)F)cc2=O)cc2c1CCC2] |
| 332 | 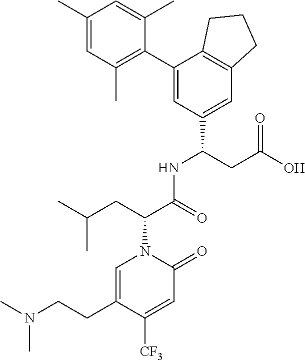 [SMILES: Cc1cc(C)c(-c2cc([C@H](CC(=O)O)NC(=O)[C@@H](CC(C)C)n3cc(CCN(C)C)c(C(F)(F)F)cc3=O)cc3c2CCC3)c(C)c1] | 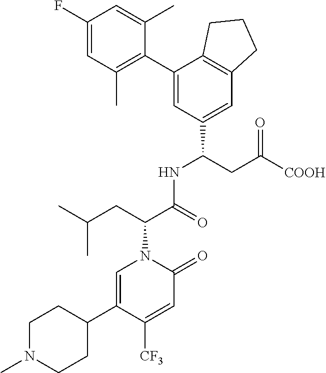 [SMILES: Cc1cc(F)cc(C)c1-c1cc([C@H](CC(=O)C(=O)O)NC(=O)[C@@H](CC(C)C)n2cc(C3CCN(C)CC3)c(C(F)(F)F)cc2=O)cc2c1CCC2] |
| 333 | 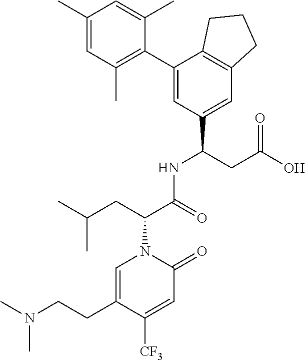 [SMILES: Cc1cc(C)c(-c2cc([C@@H](CC(=O)O)NC(=O)[C@@H](CC(C)C)n3cc(CCN(C)C)c(C(F)(F)F)cc3=O)cc3c2CCC3)c(C)c1] | 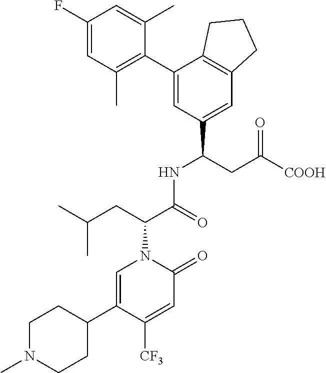 [SMILES: Cc1cc(F)cc(C)c1-c1cc([C@@H](CC(=O)C(=O)O)NC(=O)[C@@H](CC(C)C)n2cc(C3CCN(C)CC3)c(C(F)(F)F)cc2=O)cc2c1CCC2] |
| 334 | 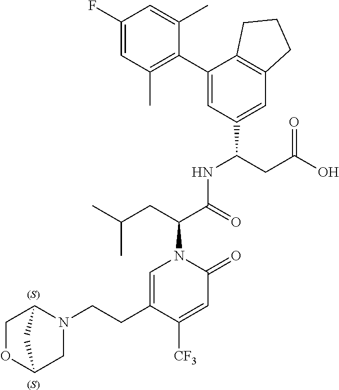 | 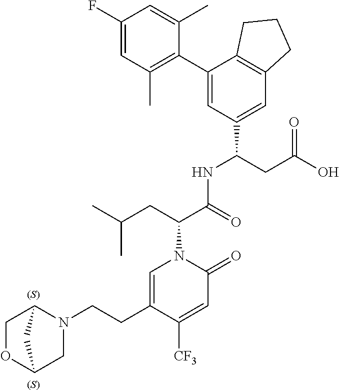 |
| 335 | | |

The present disclosure relates to a pharmaceutical composition, comprising the compound or the stereoisomer, deuterated form, solvate, prodrug, metabolite, pharmaceutically acceptable salt or co-crystal thereof according to the present disclosure, and a pharmaceutically acceptable carrier.

The present disclosure relates to the use of the compound or the stereoisomer, deuterated form, solvate, prodrug, metabolite, pharmaceutically acceptable salt or co-crystal thereof according to the present disclosure in the preparation of a drug for the treatment of diseases related to α4β7 activity or expression level, preferably the use in the preparation of a drug for inflammatory bowel diseases.

The present disclosure relates to a pharmaceutical composition or pharmaceutical preparation, comprising a therapeutically effective amount of the compound or the stereoisomer, deuterated form, solvate, prodrug, metabolite, pharmaceutically acceptable salt or co-crystal thereof according to the present disclosure, and a pharmaceutically acceptable excipient. The pharmaceutical composition can be in a unit preparation form (the amount of the drug substance in the unit preparation is also referred to as the “preparation strength”).

The present disclosure also provides a method for treating a disease in a mammal, comprising administering to the mammal a therapeutically effective amount of the compound or the stereoisomer, deuterated form, solvate, prodrug, metabolite, pharmaceutically acceptable salt or co-crystal thereof or the pharmaceutical composition according to the present disclosure. In some embodiments, the mammal according to the present disclosure comprises humans.

The term “effective amount” or “therapeutically effective amount” in the present application refers to a sufficient amount of the compound disclosed in the present application being administered which will relieve to some extent one or more of the symptoms of the disease or condition being treated, e.g., inflammatory bowel diseases. In some embodiments, the outcome is the reduction and/or remission of signs, symptoms or causes of the disease, or any other desired change in the biological system. For example, an “effective amount” in terms of the therapeutic use is an amount of the composition comprising the compound disclosed in the present application that is required to provide clinically significant reduction of the symptoms of the disease. Examples of the therapeutically effective amount includes, but are not limited to 1-1500 mg, 1-1200 mg, 1-1000 mg, 1-900 mg, 1-800 mg, 1-700 mg, 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 80-1000 mg, or 80-800 mg.

In some embodiments, the pharmaceutical composition comprises the compound or the stereoisomer, deuterated form, solvate, prodrug, metabolite, pharmaceutically acceptable salt or co-crystal thereof according to the present disclosure in an amount including, but not limited to 1-1000 mg, 20-800 mg, 40-800 mg, 40-400 mg, 25-200 mg, 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 300 mg, 320 mg, 400 mg, 480 mg, 500 mg, 600 mg, 640 mg, or 840 mg.

The present disclosure relates to a method for treating a disease in a mammal, the method comprising administering to a subject a therapeutically effective amount of the compound or the stereoisomer, deuterated form, solvate, prodrug, metabolite, pharmaceutically acceptable salt or co-crystal thereof according to the present disclosure, wherein the therapeutically effective amount is preferably 1-1500 mg, and the disease is preferably inflammatory bowel diseases.

The present disclosure relates to a method for treating a disease in a mammal, the method comprising administering to a subject a drug, i.e., the compound or the stereoisomer, deuterated form, solvate, prodrug, metabolite, pharmaceutically acceptable salt or co-crystal thereof according to the present disclosure in a daily dose of 1-1000 mg/day, wherein the daily dose can be a single dose or divided doses; in some embodiments, the daily dose includes, but is not limited to 10-1500 mg/day, 10-1000 mg/day, 10-800 mg/day, 25-800 mg/day, 50-800 mg/day, 100-800 mg/day, 200-800 mg/day, 25-400 mg/day, 50-400 mg/day, 100-400 mg/day, or 200-400 mg/day, in some embodiments, the daily dose includes, but is not limited to 10 mg/day, 20 mg/day, 25 mg/day, 50 mg/day, 80 mg/day, 100 mg/day, 125 mg/day, 150 mg/day, 160 mg/day, 200 mg/day, 300 mg/day, 320 mg/day, 400 mg/day, 480 mg/day, 600 mg/day, 640 mg/day, 800 mg/day, or 1000 mg/day.

The present disclosure relates to a kit, wherein the kit can comprise a composition in the form of a single dose or multiple doses and comprises the compound or the stereoisomer, deuterated form, solvate, prodrug, metabolite, pharmaceutically acceptable salt or co-crystal thereof according to the present disclosure, and the amount of the compound or the stereoisomer, deuterated form, solvate, prodrug, metabolite, pharmaceutically acceptable salt or co-crystal thereof according to the present disclosure is identical to the amount of same in the above-mentioned pharmaceutical composition.

In the present disclosure, the amount of the compound or the stereoisomer, deuterated form, solvate, prodrug, metabolite, pharmaceutically acceptable salt or co-crystal thereof according to the present disclosure is calculated in the form of a free base in each case.

The term “preparation strength” refers to the weight of the drug substance contained in each vial, tablet or other unit preparation.

Synthetic method of the compound according to the present disclosure:

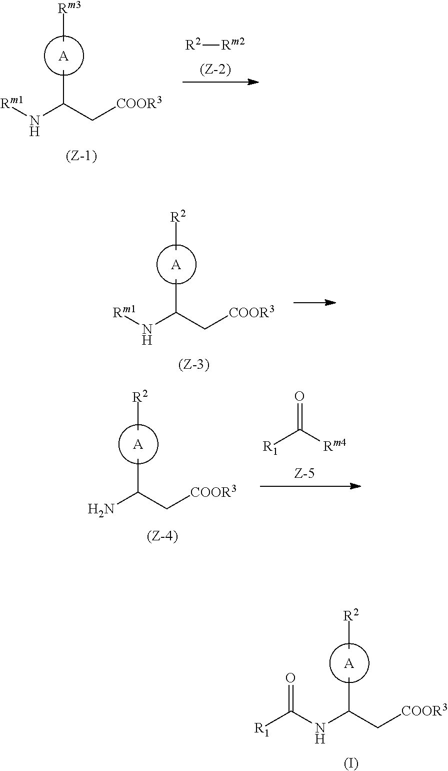

a compound of general formula (Z-1) and a compound of general formula (Z-2) are subjected to coupling or substitution reaction to obtain a compound of general formula (Z-3);
the protecting group of the compound of general formula (Z-3) is removed to obtain a compound of general formula (Z-4); and
the compound of general formula (Z-4) and a compound of general formula (Z-5) are subjected to coupling or substitution reaction to obtain the compound of general formula (I), wherein R^{m1} is selected from Boc; Cbz; tert-butylsulfinyl, etc.;
R^{m2} and R^{m3} are each independently selected from H, Cl, Br, I, OTf, B(OH)₂, boronic ester group, alkyl-substituted stannyl, etc.;
R^{m4} is selected from Cl, Br, I, OH, etc.; and
the definitions of the remaining groups are the same as those in any one of the embodiments of the above general formula (I).

Unless stated to the contrary, the terms used in the description and claims have the following meanings.

The carbon, hydrogen, oxygen, sulfur, nitrogen or F, Cl, Br, I involved in the groups and compounds of the present disclosure all comprise their isotopes, and the carbon, hydrogen, oxygen, sulfur or nitrogen involved in the groups and compounds of the present disclosure is optionally replaced with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Alkyl” refers to a linear or branched saturated aliphatic hydrocarbon group containing 1 to 20 carbon atoms, preferably alkyl containing 1 to 8 carbon atoms, more preferably alkyl containing 1 to 6 carbon atoms, further preferably alkyl containing 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof. The alkyl is optionally substituted with 0 to 6 substituents selected from F, Cl, Br, I, hydroxyl, sulfhydryl, nitro, cyano, amino, alkylamino, amido, alkenyl, alkynyl, C₁₋₆ alkyl, C₁₋₆ hydroxyalkyl, C₁₋₆ alkoxy, 3- to 8-membered carbocyclyl, 3- to 8-membered heterocyclyl, 3- to 8-membered carbocyclyloxy, 3- to 8-membered heterocyclyloxy, carboxyl or a carboxylate group, and the definition of the alkyl described herein is consistent with this definition.

“Alkylene” refers to linear and branched divalent saturated hydrocarbon groups, including —(CH₂)ᵥ— (v is an integer from 1 to 10), and examples of alkylene include, but are not limited to, methylene, ethylene, propylene, butylene, etc. The alkylene is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkylene described herein is consistent with this definition.

“Cycloalkyl” refers to a monovalent saturated carbocyclic hydrocarbon group, usually having from 3 to 12 carbon atoms, and including monocyclic cycloalkyl, fused cycloalkyl, spiro cycloalkyl or bridged cycloalkyl, and non-limiting examples include cyclopropyl, cyclobutyl, cyclopentyl, cyclohexyl, cycloheptyl, etc. The cycloalkyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The cycloalkyl described herein is as defined above.

“Alkenyl” refers to linear and branched monovalent unsaturated hydrocarbon groups having at least 1, usually 1, 2 or 3 carbon-carbon double bonds, in which the main chain comprises 2 to 10 carbon atoms, further preferably 2 to 6 carbon atoms, and more preferably 2 to 4 carbon atoms. Examples of alkenyl include, but are not limited to ethenyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene and 1,4-hexadiene. The alkenyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkenyl described herein is consistent with this definition.

“Alkynyl” refers to linear and branched monovalent unsaturated hydrocarbon groups having at least 1, usually 1, 2 or 3 carbon-carbon triple bonds, in which the main chain comprises 2 to 10 carbon atoms, further preferably 2 to 6 carbon atoms, and more preferably 2 to 4 carbon atoms. Examples of alkynyl include but are not limited to ethynyl, propargyl, 1-propynyl, 2-propynyl, 1-butynyl, 2-butynyl, 3-butynyl, 1-pentynyl, 2-pentynyl, 3-pentynyl, 4-pentynyl, 1-methyl-1-butynyl, 2-methyl-1-butynyl, 2-methyl-3-butynyl, 1-hexynyl, 2-hexynyl, 3-hexynyl, 4-hexynyl, 5-hexynyl, 1-methyl-1-pentynyl, 2-methyl-1-pentynyl, 1-heptynyl, 2-heptynyl, 3-heptynyl, 4-heptynyl, 1-octynyl, 3-octynyl, 1-nonynyl, 3-nonynyl, 1-decynyl, 4-decynyl, etc. The alkynyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkynyl described herein is consistent with this definition.

“Alkoxy” refers to —O-alkyl. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy. The alkoxy is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkoxy described herein is consistent with this definition.

“Carbocyclyl” or “carbocycle” refers to a substituted or unsubstituted saturated or unsaturated aromatic ring or non-aromatic ring, wherein the aromatic ring or non-aromatic ring can be a 3- to 8-membered monocyclic ring, a 4- to 12-membered bicyclic ring or a 10- to 15-membered tricyclic ring system. Carbocyclyl can be connected to an aromatic ring or a non-aromatic ring, wherein the aromatic ring or non-aromatic ring is optionally a monocyclic ring, a bridged ring or a spiro ring. Non-limiting examples include cyclopropane, cyclobutane, cyclopentane, cyclohexane, cycloheptane, 1-cyclopentyl-1-enyl, 1-cyclopentyl-2-enyl, 1-cyclopentyl-3-enyl, cyclohexyl, 1-cyclohexyl-2-enyl, 1-cyclohexyl-3-enyl, cyclohexenyl, a benzene ring, a naphthalene ring

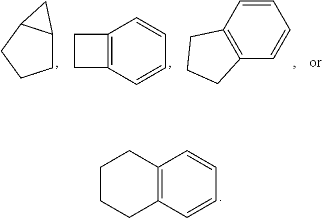

The carbocycle is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group. The definition of the carbocycle or carbocyclyl described herein is consistent with this definition.

“Heterocyclyl” or “heterocycle” refers to a substituted or unsubstituted saturated or unsaturated aromatic ring or non-aromatic ring, and the aromatic ring or non-aromatic ring can be a 3- to 8-membered monocyclic ring, a 4- to 12-membered bicyclic ring or a 10- to 15-membered tricyclic ring system, and contains 1 to 3 heteroatoms selected from N, O or S, preferably 3- to 8-membered heterocyclyl, and the optionally substituted N, S in the ring of heterocyclyl can be oxidized into various oxidation states. Heterocyclyl can be connected to a heteroatom or a carbon atom; heterocyclyl can be connected to an aromatic ring or a non-aromatic ring; and heterocyclyl can be connected to a bridged ring or a spiro ring. Non-limiting examples include oxiranyl, aziridinyl, oxetanyl, azetidinyl, 1,3-dioxolanyl, 1,4-dioxolanyl, 1,3-dioxanyl, azacycloheptyl, pyridyl, furyl, thienyl, pyranyl, N-alkylpyrrolyl, pyrimidyl, pyrazinyl, pyridazinyl, imidazolyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, tetrahydrothiazolyl, tetrahydropyranyl, benzoimidazolyl, benzopyridyl, pyrrolopyridyl, benzodihydrofuryl, pyrrolyl, pyrazolyl, thiazolyl, oxazolyl, pyrazinyl, indazolyl, benzothienyl, benzofuryl, benzopyrrolyl, benzoimidazolyl, benzothiazolyl, benzoxazolyl, benzopyridyl, benzopyrimidyl, benzopyrazinyl, piperazinyl, azabicyclo[3.2.1]octanyl, azabicyclo[5.2.0]nonanyl, oxatricyclo[5.3.1.1]dodecyl, azaadamantyl, oxaspiro[3.3]heptanyl,

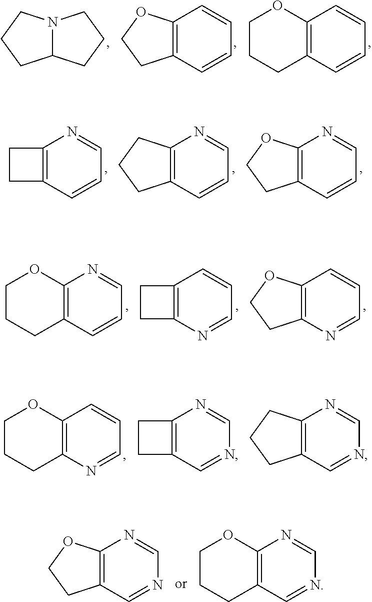

The heterocyclyl is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group. The definition of the heterocyclyl described herein is consistent with this definition.

“Heterocycloalkyl” refers to substituted or unsubstituted saturated heterocyclyl, and can be a 3- to 8-membered monocyclic ring, a 4- to 12-membered bicyclic ring or a 10- to 15-membered tricyclic ring system, and contains 1 to 3 heteroatoms selected from N, O or S, preferably 3- to 8-membered heterocyclyl, and the optionally substituted N, S in the ring of heterocycloalkyl can be oxidized into various oxidation states. Heterocycloalkyl can be connected to a heteroatom or a carbon atom. Non-limiting examples include oxiranyl, aziridinyl, oxetanyl, azetidinyl, 1,3-dioxolanyl, 1,4-dioxolanyl, 1,3-dioxanyl, azacycloheptyl, piperidyl, or morpholinyl. The heterocycloalkyl is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group. The definition of the heterocycloalkyl described herein is consistent with this definition.

“Spiro ring” refers to a 5- to 20-membered polycyclic group sharing one atom (referred to as a spiro atom) between substituted or unsubstituted monocyclic rings, which may contain 0 to 5 double bonds, and may contain 0 to 5 heteroatoms selected from N, O or S(═O)n (n is selected from 0, 1 or 2). The spiro ring is preferably 6- to 14-membered, further preferably 6- to 12-membered, and more preferably 6- to 10-membered. Its non-limiting examples include:

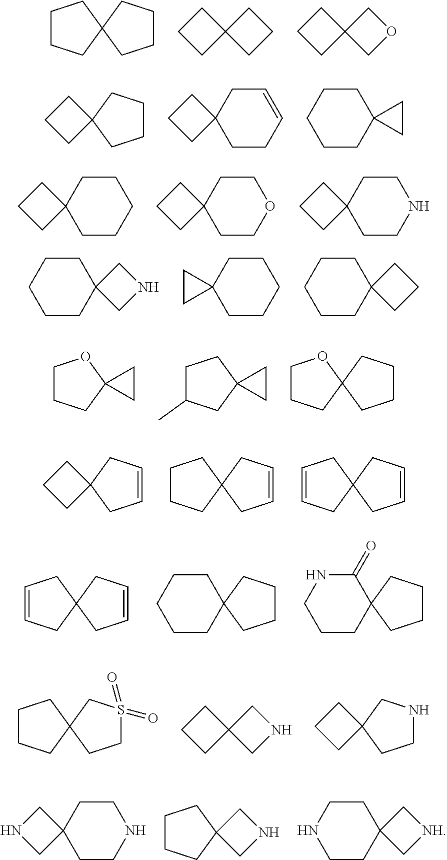

When the spiro ring is substituted, substituents can be 1 to 5 groups selected from F, Cl, Br, I, alkyl, cycloalkyl, alkoxy, haloalkyl, thiol, hydroxyl, nitro, sulfhydryl, amino, cyano, isocyano, aryl, heteroaryl, heterocyclyl, bridged ring group, spiro ring group, fused ring group, hydroxyalkyl, ═O, carbonyl, aldehyde, carboxylic acid, formate, —(CH₂)ₘ—C(═O)—Ra, —O—(CH₂)ₘ—C(═O)—Ra, —(CH₂)ₘ—C(═O)—NRbRc, —(CH₂)ₘS(═O)nRa, —(CH₂)ₘ-alkenyl-Ra, ORd or —(CH₂)ₘ-alkynyl-Ra (wherein m and n are 0, 1 or 2), arylthio, thiocarbonyl, silyl or —NRbRc and the like, wherein Rb and Rc are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, Rb and Rc may form a five- or six-membered cycloalkyl or heterocyclyl. Ra and Rd are each independently selected from aryl, heteroaryl, alkyl, alkoxy, cycloalkyl, heterocyclyl, carbonyl, ester group, bridged ring group, spiro ring group or fused ring group. The definition of the spiro ring described herein is consistent with this definition.

“Fused ring” refers to a polycyclic group in which each ring in the system shares an adjacent pair of atoms with other rings in the system, wherein one or more of the rings may contain 0 or more double bonds, which may be substituted or unsubstituted, and each ring in the fused ring system may contain 0 to 5 heteroatoms selected from N, S(═O)n or O (n is selected from 0, 1 or 2). The fused ring is preferably 5- to 20-membered, further preferably 5- to 14-membered, more preferably 5- to 12-membered, and still further preferably 5- to 10-membered. Non-limiting examples include:

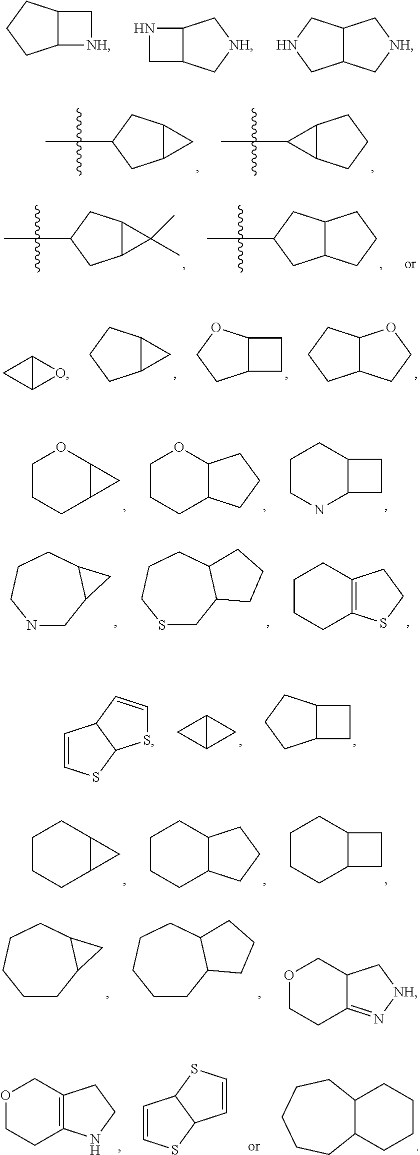

When the fused ring is substituted, substituents can be 1 to 5 groups selected from F, Cl, Br, I, alkyl, cycloalkyl, alkoxy, haloalkyl, thiol, hydroxyl, nitro, sulfhydryl, amino, cyano, isocyano, aryl, heteroaryl, heterocyclyl, bridged ring group, spiro ring group, fused ring group, hydroxyalkyl, ═O, carbonyl, aldehyde group, carboxylic acid group, formate group, —(CH₂)ₘ—C(═O)—Ra, —O—(CH₂)ₘ—C(═O)—Ra, —(CH₂)ₘ—C(═O)—NRbRc, —(CH₂)ₘS(═O)ₙRa, —(CH₂)ₘ-alkenyl-Ra, ORd or —(CH₂)ₘ-alkynyl-Ra (wherein m and n are 0, 1 or 2), arylthio, thiocarbonyl, silyl or —NRbRc and the like, wherein Rb and Rc are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, Rb and Rc may form a five- or six-membered cycloalkyl or heterocyclyl. Ra and Rd are each independently selected from aryl, heteroaryl, alkyl, alkoxy, cycloalkyl, heterocyclyl, carbonyl, ester group, bridged ring group, spiro ring group or fused ring group. The definition of the fused ring described herein is consistent with this definition.

“Bridged ring” refers to a polycyclic group containing any two atoms that are not directly connected, which group may contain 0 or more double bonds and can be substituted or unsubstituted, and any ring in the fused ring system may contain 0 to 5 heteroatoms or groups selected from N, S(═O)n or O (wherein n is 0, 1 or 2). The ring atoms contain 5 to 20 atoms, preferably 5 to 14 atoms, further preferably 5 to 12 atoms, and still further preferably 5 to 10 atoms. Non-limiting examples include

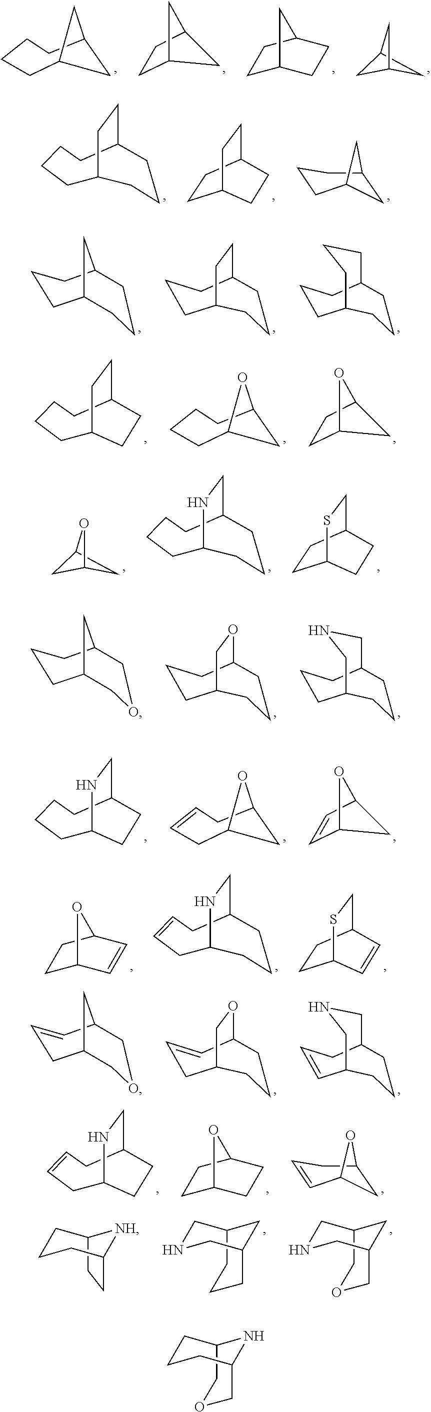

and adamantane. When the bridged ring is substituted, substituents can be 1 to 5 groups selected from F, Cl, Br, I, alkyl, cycloalkyl, alkoxy, haloalkyl, thiol group, hydroxyl, nitro, sulfhydryl, amino, cyano, isocyano, aryl, heteroaryl, heterocyclyl, bridged ring group, spiro ring group, fused ring group, hydroxyalkyl, ═O, carbonyl, aldehyde group, carboxylic acid group, formate group, —(CH₂)ₘ—C(═O)—Ra, —O—(CH₂)ₘ—C(═O)—Ra, —(CH₂)ₘ—C(═O)—NRbRc, —(CH₂)ₘS(═O)ₙRa, —(CH₂)ₘ-alkenyl-Ra, ORd or —(CH₂)ₘ-alkynyl-Ra (wherein m and n are 0, 1 or 2), arylthio, thiocarbonyl, silyl or —NRbRc and the like, wherein Rb and Rc are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, Rb and Rc may form a five- or six-membered cycloalkyl or heterocyclyl. Ra and Rd are each independently selected from aryl, heteroaryl, alkyl, alkoxy, cycloalkyl, heterocyclyl, carbonyl, ester group, bridged ring group, spiro ring group or fused ring group. The definition of the bridged ring described herein is consistent with this definition.

“Aryl” or “aromatic ring” refers to a monovalent aromatic hydrocarbon group with a monocyclic ring or a fused ring, which generally has 6 to 12 carbon atoms and can be substituted or unsubstituted. When the aryl or aromatic ring is substituted, substituents can be 1 to 5 groups selected from F, Cl, Br, I, alkyl, cycloalkyl, alkoxy, haloalkyl, thiol group, hydroxyl, nitro, sulfhydryl, amino, cyano, isocyano, aryl, heteroaryl, heterocyclyl, bridged ring group, spiro ring group, fused ring group, hydroxyalkyl, ═O, carbonyl, aldehyde group, carboxylic acid group, formate group, —(CH₂)ₘ—C(═O)—Ra, —O—(CH₂)ₘ—C(═O)—Ra, —(CH₂)ₘ—C(═O)—NRbRc, —(CH₂)ₘS(═O)ₙRa, —(CH₂)ₘ-alkenyl-Ra, ORd or —(CH₂)ₘ-alkynyl-Ra (wherein m and n are 0, 1 or 2), arylthio, thiocarbonyl, silyl or —NRbRc and the like, wherein Rb and Rc are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, Rb and Rc may form a five- or six-membered cycloalkyl or heterocyclyl. Ra and Rd are each independently selected from aryl, heteroaryl, alkyl, alkoxy, cycloalkyl, heterocyclyl, carbonyl, ester group, bridged ring group, spiro ring group or fused ring group. The definition of the aryl or aromatic ring described herein is consistent with this definition.

“Heteroaryl” refers to a substituted or unsubstituted 5- to 15-membered aromatic ring, contains 1 to 5 heteroatoms or groups selected from N, O or S(═O)ₙ, and is preferably a 5- to 10-membered heteraromatic ring, further preferably a 5- to 6-membered heteraromatic ring. Non-limiting examples of heteroaryl include, but are not limited to pyridyl, furyl, thienyl, pyranyl, N-alkylpyrrolyl, pyrimidyl, pyrazinyl, pyridazinyl, imidazolyl, benzopyrazolyl, benzoimidazolyl, benzopyridyl, pyrrolopyridyl, etc. The heteroaryl ring can be fused to an aryl, heterocyclyl or cycloalkyl ring, wherein the ring connected to the parent structure is a heteroaryl ring, and non-limiting examples include

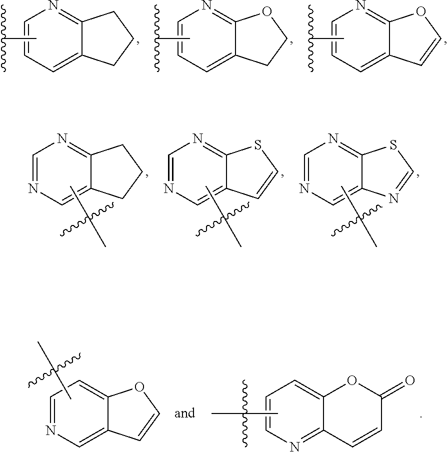

When the heteroaryl is substituted, substituents can be 1 to 5 groups selected from F, Cl, Br, I, alkyl, cycloalkyl, alkoxy, haloalkyl, thiol, hydroxyl, nitro, sulfhydryl, amino, cyano, isocyano, aryl, heteroaryl, heterocyclyl, bridged ring group, spiro ring group, fused ring group, hydroxyalkyl, ═O, carbonyl, aldehyde, carboxylic acid, formate, —(CH₂)ₘ—C(═O)—Ra, —O—(CH₂)ₘ—C(═O)—Ra, —(CH₂)ₘ—C(═O)—NRbRc, —(CH₂)ₘS(═O)ₙRa, —(CH₂)ₘ-alkenyl-Ra, ORd or —(CH₂)ₘ-alkynyl-Ra (wherein m and n are 0, 1 or 2), arylthio, thiocarbonyl, silyl or —NRbRc and the like, wherein Rb and Rc are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, Rb and Rc may form a five- or six-membered cycloalkyl or heterocyclyl. Ra and Rd are each independently selected from aryl, heteroaryl, alkyl, alkoxy, cycloalkyl, heterocyclyl, carbonyl, ester group, bridged ring group, spiro ring group or fused ring group. The definition of the heteroaryl described herein is consistent with this definition.

“Containing 1 to 4 heteroatoms selected from O, S or N” refers to the circumstance where 1, 2, 3 or 4 heteroatoms selected from O, S or N may be contained.

“Substituted with 0 to X substituents” refers to the circumstance where the specified entity is substituted with 0, 1, 2, 3 . . . X substituents, wherein X is selected from any integer between 1 and 10. For example, “substituted with 0 to 4 substituents” refers to the circumstance where the specified entity is substituted with 0, 1, 2, 3 or 4 substituents. For example, “substituted with 0 to 5 substituents” refers to the circumstance where the specified entity is substituted with 0, 1, 2, 3, 4 or 5 substituents. For example, “bridged-heterocyclic ring is optionally substituted with 0 to 4 substituents selected from H or F” means that the bridged-heterocyclic ring is optionally substituted with 0, 1, 2, 3 or 4 substituents selected from H or F.

An X- to Y-membered ring (X is selected from an integer less than Y and greater than or equal to 3, and Y is selected from any integer between 4 and 12) includes X+1-, X+2-, X+3-, X+4-, . . . , Y-membered rings. Rings include heterocycle, carbocycle, an aromatic ring, aryl, heteroaryl, cycloalkyl, a mono-heterocyclic ring, a fused-heterocyclic ring, a spiro-heterocyclic ring or a bridged-heterocyclic ring. For example, a “4- to 7-membered mono-heterocyclic ring” refers to a 4-, 5-, 6- or 7-membered mono-heterocyclic ring, and a “5- to 10-membered fused-heterocyclic ring” refers to a 5-, 6-, 7-, 8-, 9- or 10-membered fused-heterocyclic ring.

The term “optional” or “optionally” refers to that the events or circumstances subsequently described may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

“Pharmaceutically acceptable salt” or “pharmaceutically acceptable salt thereof” refers to a salt of the compound according to the present disclosure, which salt maintains the biological effectiveness and characteristics of a free acid or a free base, and is obtained by reacting the free acid with a non-toxic inorganic base or organic base, or reacting the free base with a non-toxic inorganic acid or organic acid.

“Pharmaceutical composition” refers to a mixture of one or more of the compounds of the present disclosure, a pharmaceutically acceptable salt or a prodrug thereof, and other chemical components, wherein “other chemical components” refer to pharmaceutically acceptable carriers, excipients and/or one or more other therapeutic agents.

“Carrier” refers to a material that does not cause significant irritation to an organism and does not eliminate the biological activity and characteristics of a compound administered.

“Animal” is meant to include mammals, such as humans, companion animals, zoo animals, and domestic animals, preferably humans, horses, or dogs.

The term “stereoisomer” refers to an isomer produced as a result of different spatial arrangement of atoms in molecules, including cis-trans isomers, enantiomers and conformational isomers.

### DETAILED DESCRIPTION OF EMBODIMENTS

The technical solutions of the present disclosure will be described in detail by the following examples, but the scope of protection of the present disclosure includes but is not limited thereto.

To achieve the objectives of the present disclosure, according to organic synthesis techniques known to those skilled in the art, and starting from commercially available chemicals and/or compounds described in chemical documents, the prepared compounds, “commercially available chemicals”, for use in the reactions described herein are obtained from standard commercial sources, including Shanghai Aladdin Bio-Chem Technology Co., Ltd., Shanghai Macklin Biochemical Co., Ltd., Sigma-Aldrich, Alfa Aesar (China) Chemical Co., Ltd., Tokyo Chemical Industry (Shanghai) Co., Ltd., Energy Chemical Co., Ltd., Shanghai Titan Scientific Co., Ltd., Kelong Chemical Co., Ltd., J&K Scientific and the like.

The structures of the compounds are determined by nuclear magnetic resonance (NMR) or (and) mass spectrometry (MS). The NMR shift (δ) is given in the unit of 10-6 (ppm). NMR is determined with Bruker Avance III 400 and Bruker Avance 300; the solvent for determination is deuterated dimethyl sulfoxide (DMSO-d6), deuterated chloroform (CDCl₃) and deuterated methanol (CD₃OD); and the internal standard is tetramethylsilane (TMS).

MS is determined with Agilent 6120B (ESI) and Agilent 6120B (APCI);
HPLC is determined with Agilent 1260DAD high pressure liquid chromatograph (Zorbax SB-C18 100×4.6 mm, 3.5 μM);
Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
and for the column chromatography, Yantai Huanghai silica gel of 200-300 mesh silica gel is generally used as a carrier.
Boc: tert-butoxycarbonyl; Ts: p-toluenesulfonyl; Cbz: benzyloxycarbonyl; TMS: trimethylsilyl;
TIPS: triisopropylsilyl; Bpin: pinacol boronate; MOM: methoxymethyl ethers; HOBT: 1-hydroxybenzotriazole; EDCI: 1-(3-dimethylaminopropyl)-3-ethylcarbodiimide hydrochloride
HATU: 2-(7-azabenzotriazole)-N,N,N′,N′-tetramethyluronium hexafluorophosphate.
* next to a chemical bond represents that the chirality of the chiral atom is R or S; unless otherwise specified, synthesis is carried out at room temperature.

### Intermediate 1: 4-methyl-2-(4-methyl-2-oxopyridin-1(2H)-yl)pentanoic acid

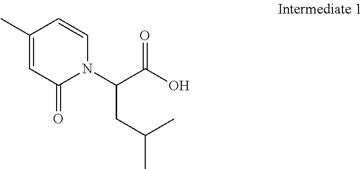 [SMILES: Cc1ccn(C(CC(C)C)C(=O)O)c(=O)c1]

Magnesium tert-butoxide (1.75 g, 10.26 mmol) and potassium tert-butoxide (580 mg, 5.13 mmol) were added to THF (30 mL), and at 0° C., 2-hydroxy-4methylpyridine (1 g, 5.13 mmol) was added. The mixture was stirred for 20 min, 2-bromo-4-methylpentanoic acid (840 mg, 7.7 mmol) was added, and the resulting mixture was stirred at room temperature for 40 h. The reaction mixture was adjusted with 3N hydrochloric acid to pH=4, and then extracted with ethyl acetate (150 mL×3). The organic phases were combined and dried over anhydrous sodium sulfate and the filtrate was concentrated under reduced pressure and purified by reverse phase column to afford intermediate 1 (501 mg, yield: 43.74%).

Ms m/z (ESI): 224.3 [M+H]⁺.

### Example 1: Preparation of Compound 1

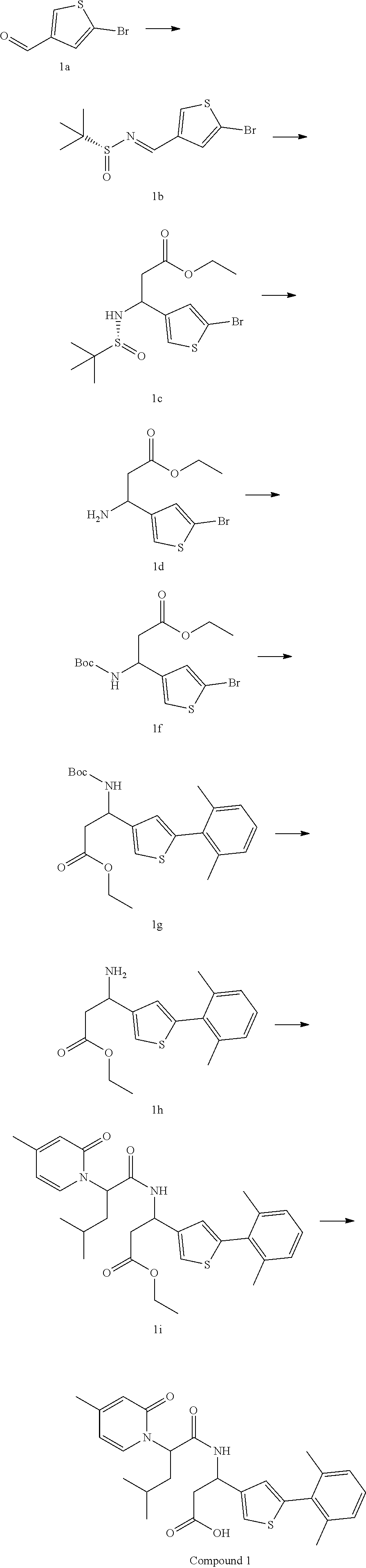

### Step 1: Synthesis of 1b

1a (9.0 g, 47.11 mmol) was dissolved in THF (150 mL), (R)-(+)-tert-butylsulfinamide (8.56 g, 70.66 mmol) and tetraethyl titanate (21.49 g, 94.22 mmol) were successively added, and the mixture was stirred at 85° C. under nitrogen protection for 4 h. The reaction mixture was cooled to room temperature, and concentrated under reduced pressure to afford a residue, which was then subjected to silica gel column chromatography (petroleum ether:ethyl acetate=5:1) to afford 1b (12.5 g, yield: 90.18%).

LC-Ms m/z (ESI): 294.1 [M+H]⁺.

### Step 2: Synthesis of 1c

Zinc powder (11.75 g, 179.64 mmol) was added to anhydrous THF (100 mL). The reaction system was subjected to nitrogen replacement three times, and at 0° C., bromine (0.48 g, 2.99 mmol) was slowly added. The mixture was stirred for 10 min and at 50° C., ethyl bromoacetate (10.0 g, 59.88 mmol) was added. The resulting mixture was stirred for 4 h and then cooled to room temperature. A solution of 1b (9.0 g, 47.11 mmol) in THF (50 mL) was slowly added. The reaction mixture was then stirred at 25° C. under nitrogen protection for 2 h. The reaction liquid was concentrated under reduced pressure and subjected to silica gel column chromatography (petroleum ether:ethyl acetate=4:1) to afford 1c (7.1 g, yield: 54.63%).

LC-Ms m/z (ESI): 382.2 [M+H]⁺.

### Step 3: Synthesis of 1d

1c (7.0 g, 18.31 mmol) was dissolved in THF (150 mL), hydrogen chloride 1,4-dioxane solution (4N, 10 mL) was added, and the mixture was stirred at room temperature for 2 h. After the reaction system was adjusted to pH=8 with saturated sodium bicarbonate, the mixture was diluted with water (100 mL), and extracted with ethyl acetate (200 mL×3). The organic phases were combined, dried over anhydrous sodium sulfate and filtered, and the filtrate was concentrated under reduced pressure to afford crude product 1d.

### Step 4: Synthesis of 1f

1d (4.5 g, 16.18 mmol) was dissolved in THF (150 mL) and water (50 mL), sodium carbonate (5.14 g, 48.54 mmol) and di-tert-butyl dicarbonate (4.36 g, 20 mmol) were added, and the mixture was stirred at room temperature for 12 h. The reaction system was diluted with water (100 mL) and extracted with ethyl acetate (150 mL×3). The organic phases were combined, dried over anhydrous sodium sulfate and filtered. The filtrate was concentrated under reduced pressure and subjected to silica gel column chromatography (petroleum ether:ethyl acetate=5:1) to afford 1f (5.1 g, 83.3%).

### Step 5: Synthesis of 1g

Under nitrogen protection, 1f (1 g, 2.64 mmol), (2,6-dimethylphenyl)boronic acid (480.0 mg, 3.17 mmol), Pd(dppf)Cl₂·DCM (220.0 mg, 0.26 mmol) and cesium carbonate (2.58 g, 7.92 mmol) were added to 1,4-dioxane (30.0 mL) and water (6.0 mL), and the mixture was stirred at 100° C. for 5 h.

After the reaction mixture was cooled to room temperature, the solvent was removed under reduced pressure. Water (30 mL) was added, and the resulting mixture was extracted with ethyl acetate (20 mL×3). The organic phases were combined, dried over anhydrous sodium sulfate and filtered. The filtrate was concentrated under reduced pressure, and the residue was subjected to silica gel column chromatography (PE:EA=4:1) to afford 1g (810 mg, yield: 76%).

### Step 6: Synthesis of 1h

1g (800 mg, 1.98 mmol) was dissolved in dichloromethane (10 mL), TFA (2 mL) was added, and the mixture was stirred at room temperature for 2 h. The reaction mixture was concentrated under reduced pressure to afford 1h (450 mg, yield: 74.9%).

Ms m/z (ESI): 304.2[M+H]⁺.

### Step 7: Synthesis of 1i

Intermediate 1 (180 mg, 0.81 mmol) was dissolved in DCM (8 mL). At 0° C., oxalyl chloride (410 mg, 3.24 mmol) and DMF (0.05 mL) were added, and the reaction mixture was stirred for 1 h and concentrated under reduced pressure to afford residue 1. 1h (220 mg, 0.74 mmol) and DIPEA (380 mg, 2.96 mmol) were dissolved in DCM (8 mL). At 0° C., residue 1 was added and the resulting mixture was stirred at 25° C. for 12 h. The reaction liquid was concentrated under reduced pressure and subjected to silica gel column chromatography (petroleum ether:ethyl acetate=1:1) to afford 1i (280 mg, yield: 74.39%).

LC-Ms m/z (ESI): 509.3[M+H]⁺.

### Step 8: Synthesis of Compound 1

1i (280 mg, 0.55 mmol) was dissolved in acetonitrile (10 mL) and water (2 mL), 1,5,7-triazidobicyclo(4.4.0)dec-5-ene (280 mg, 0.55 mmol) was added, and the mixture was stirred at room temperature for 3 h. The reaction mixture was concentrated under reduced pressure and the residue was subjected to silica gel column chromatography to afford compound 1 (213 mg, 80.6%).

LC-Ms m/z (ESI): 481.1[M+H]⁺.

### Example 2: Preparation of Compound 2 and Stereoisomer Thereof

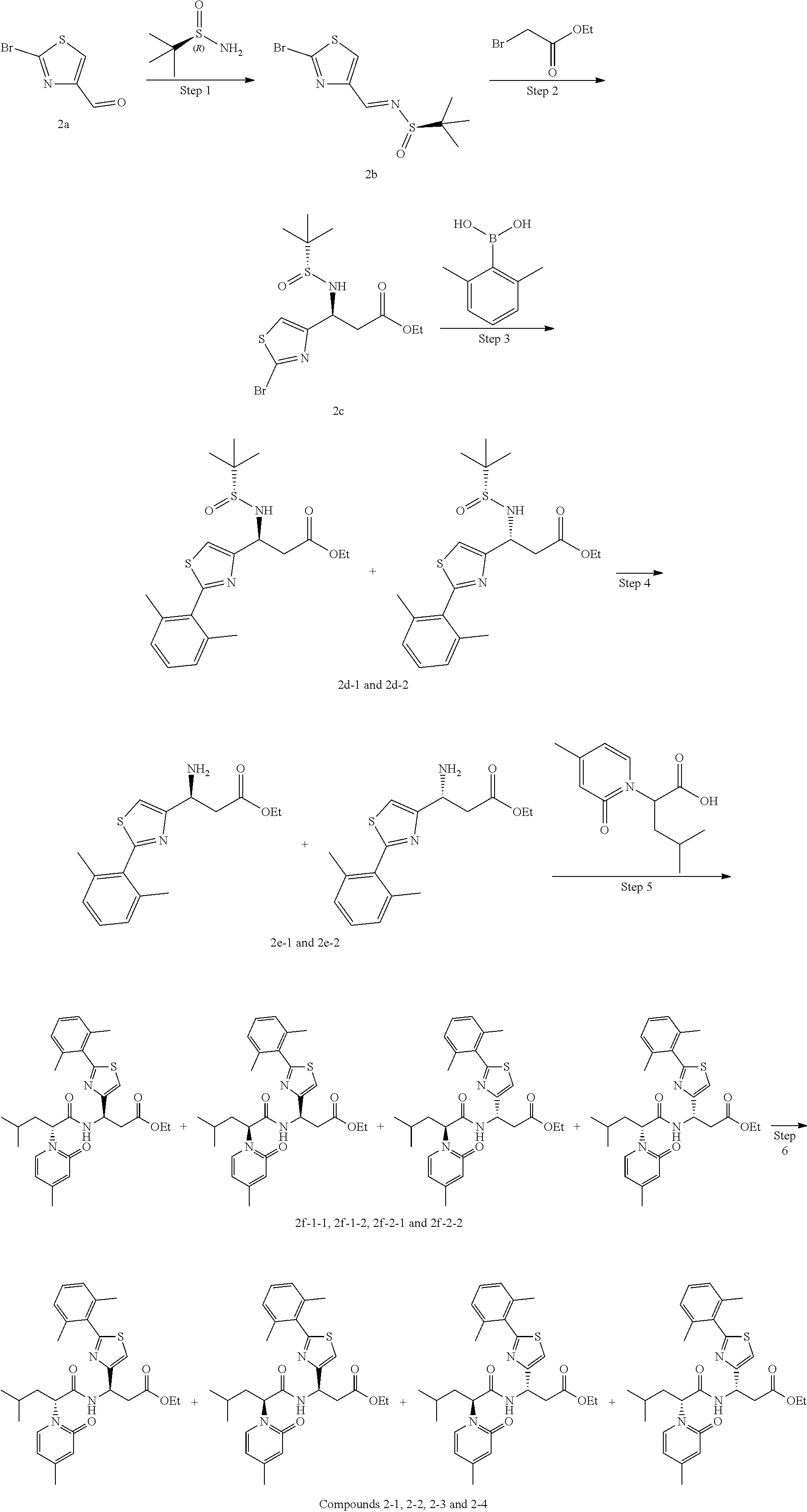

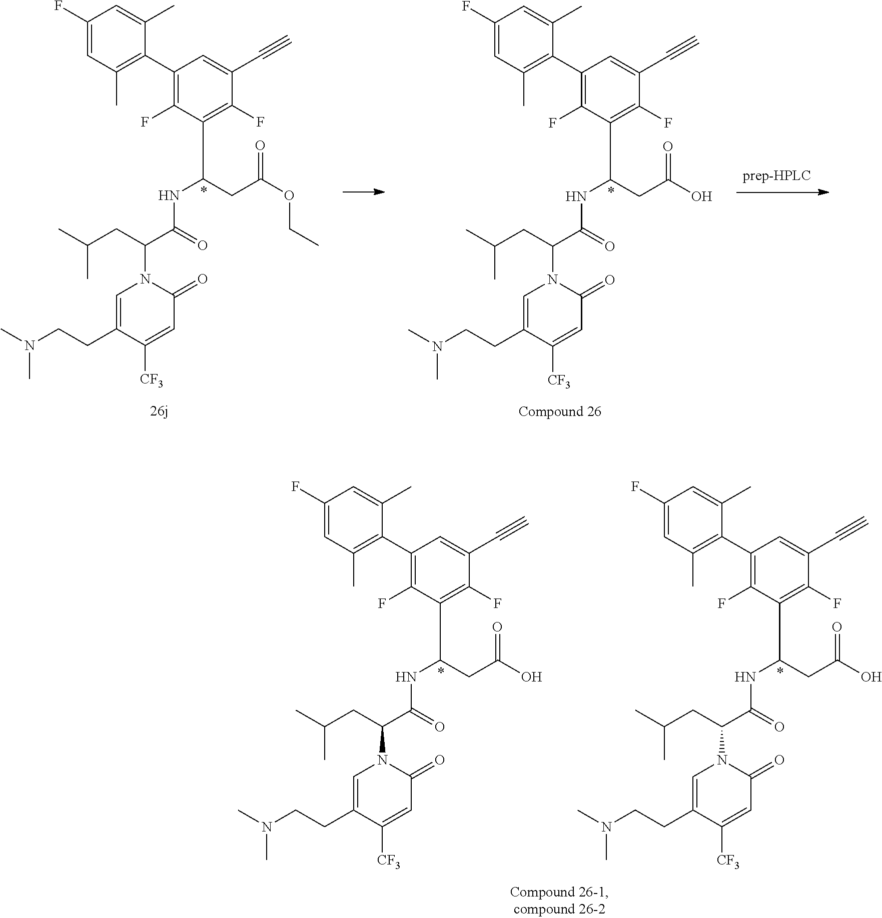 [SMILES: C#Cc1cc(-c2c(C)cc(F)cc2C)c(F)c(C(CC(=O)OCC)NC(=O)C(CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)C(CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)[C@H](CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)[C@@H](CC(C)C)n2cc(CCN(C)C)c(C(F)(F)F)cc2=O)c1F]

For the trifluoroacetate of compound 2-1 (45.0 mg), trifluoroacetate of compound 2-2 (60.0 mg), trifluoroacetate of compound 2-3 (63.0 mg) and trifluoroacetate of compound 2-4 (86.0 mg), reference can be made to example 1 for the reaction conditions and operations.

Preparative HPLC conditions for the final product: instrument: waters 2767 preparative chromatographic column: SunFire@Prep C18 (19 mm×150 mm); composition of mobile phases: mobile phase A: acetonitrile, and mobile phase B: water (containing 0.1% TFA).

Compound 2-1: LC-Ms m/z (ESI): 482.3 [M+H]⁺.

¹H NMR (400 MHz, DMSO-d₆) δ 8.92 (d, 1H), 7.58 (d, 1H), 7.50 (s, 1H), 6.90 (d, 1H), 7.27 (t, 1H), 7.15 (d, 2H), 6.19 (s, 1H), 6.12-6.05 (m, 1H), 5.59-5.52 (m, 1H), 5.40-5.33 (m, 1H), 2.96-2.87 (m, 1H), 2.80-2.69 (m, 1H), 2.21 (s, 3H), 2.04 (s, 6H), 1.91-1.81 (m, 1H), 1.79-1.69 (m, 1H), 1.39-1.26 (m, 1H), 0.94-0.76 (m, 6H).

Compound 2-2: LC-Ms m/z (ESI): 482.3 [M+H]⁺.

¹H NMR (400 MHz, DMSO-d₆) δ 8.92 (d, 1H), 7.59 (d, 1H), 7.55 (s, 1H), 7.29 (t, 1H), 6.90 (d, 1H), 7.17 (d, 2H), 6.19 (s, 1H), 6.12-6.05 (m, 1H), 5.68-5.59 (m, 1H), 5.39-5.29 (m, 1H), 2.93-2.83 (m, 1H), 2.81-2.71 (m, 1H), 2.12 (s, 3H), 2.07 (s, 6H), 1.87-1.76 (m, 1H), 1.70-1.60 (m, 1H), 1.27-1.16 (m, 1H), 0.85-0.75 (m, 6H).

Compound 2-3: LC-Ms m/z (ESI): 482.3 [M+H]⁺.

¹H NMR (400 MHz, DMSO-d₆) δ 8.91 (d, 1H), 7.58 (d, 1H), 7.50 (d, 1H), 7.27 (t, 1H), 7.15 (d, 2H), 6.19 (s, 1H), 6.11-6.07 (m, 1H), 5.59-5.52 (m, 1H), 5.40-5.33 (m, 1H), 2.96-2.87 (m, 1H), 2.79-2.70 (m, 1H), 2.11 (s, 3H), 2.04 (s, 6H), 1.92-1.82 (m, 1H), 1.79-1.69 (m, 1H), 1.39-1.27 (m, 1H), 0.91-0.79 (m, 6H).

Compound 2-4: LC-Ms m/z (ESI): 482.3 [M+H]⁺.

¹H NMR (400 MHz, DMSO-d₆) δ 8.89 (d, 1H), 7.59 (d, 1H), 7.54 (d, 1H), 7.28 (t, 1H), 7.16 (d, 2H), 6.19 (s, 1H), 6.11-6.06 (m, 1H), 5.67-5.60 (m, 1H), 5.39-5.31 (m, 1H), 2.93-2.83 (m, 1H), 2.81-2.71 (m, 1H), 2.12 (s, 3H), 2.07 (s, 6H), 1.87-1.76 (m, 1H), 1.71-1.61 (m, 1H), 1.27-1.15 (m, 1H), 0.85-0.73 (m, 6H).

### Example 3: Preparation of Compound 3 and Stereoisomer Thereof

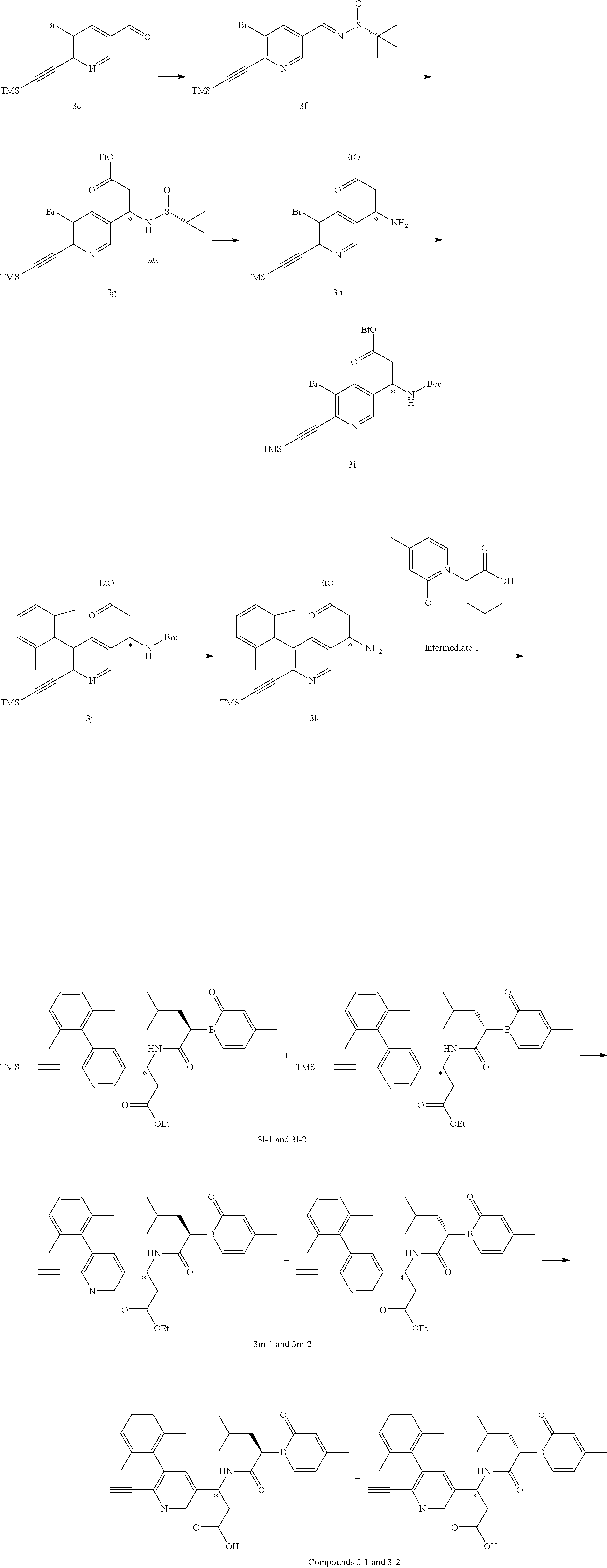

### Step 1: Synthesis of 3f

3e (see Journal of the American Chemical Society (2020), 142 (43), 18387-18406 for the preparation method) (6.5 g, 23.0 mmol) was dissolved in THF (100 mL). Under ice bath conditions, (R)-(+)-tert-butylsulfinamide (3.4 g, 27.6 mmol) and tetraethyl titanate (13.1 g, 57.6 mmol) were successively added and the resulting mixture was stirred at 55° C. under nitrogen protection for 3 h. The reaction mixture was cooled to room temperature, followed by the addition of ice water (100 mL). The mixture was filtered and the filter cake was washed with ethyl acetate (20 mL×2). The filtrate was collected and allowed to stand for layering. The organic phase was collected and the aqueous phase was extracted with ethyl acetate (30 mL×3). The organic phases were combined, dried over anhydrous sodium sulfate, filtered and concentrated to afford a crude, which was subjected to silica gel column chromatography to afford product 3f (8.6 g, yield: 96.9%).

LC-Ms m/z (ESI): 385.0 [M+H]⁺.

### Step 2: Synthesis of 3g

Zinc powder (20.2 g, 308.0 mmol) was added to anhydrous THF (180 mL), CuCl (6.5 g, 66.0 mmol) was added, and the mixture was subjected to nitrogen replacement three times and stirred at 60° C. for 1 h. At 0° C., a solution of ethyl bromoacetate (18.4 g, 110.0 mmol) in THF (10 mL) was slowly added dropwise and the mixture was stirred at 60° C. for 1 h. At 0° C., a solution of 3f (8.5 g, 22.0 mmol) in THF (10 mL) was slowly added dropwise and the mixture was stirred at 0° C. for 3 h. The reaction mixture was filtered and the filtrate was quenched with 1 N HCl, and extracted with ethyl acetate (50 mL×3). The organic phases were combined, dried over anhydrous sodium sulfate, filtered and concentrated and the residue was purified by silica gel column to afford 3g (8.2 g, yield: 78.7%).

LC-Ms m/z (ESI): 473.1 [M+H]⁺.

### Step 3: Synthesis of 3h

3g (1.8 g, 3.8 mmol) was dissolved in THF (12 mL), 4 N hydrogen chloride 1,4-dioxane solution (12 mL) was added, and the mixture was stirred at room temperature for 2 h. The reaction system was concentrated under reduced pressure to afford crude 3h.

LC-Ms m/z (ESI): 369.0[M+H]⁺.

### Step 4: Synthesis of 3i

3h (1.4 g, 3.5 mmol) was dissolved in THF (15 mL) and water (20 mL), sodium carbonate (740.0 mg, 6.9 mmol) was added, a solution of di-tert-butyl dicarbonate (830.0 mg, 3.8 mmol) in THF (5 mL) was added dropwise, and the mixture was stirred at room temperature for 3 h. The reaction mixture was extracted with ethyl acetate (20 mL×2), and the organic phases were combined, dried over anhydrous sodium sulfate, filtered and concentrated and the residue was subjected to silica gel column chromatography to afford 3i (1.1 g, yield: 69.4%).

LC-Ms m/z (ESI): 469.0[M+H]⁺.

### Step 5: Synthesis of 3j

Under nitrogen protection, 3i (910.0 mg, 1.9 mmol) was dissolved in 1,4-dioxane (20 mL) and water (2 mL), 2,6-dimethyl phenylboronic acid (580.0 mg, 3.9 mmol), Pd(dppf)Cl₂ (160.0 mg, 0.2 mmol) and cesium carbonate (1.9 g, 5.8 mmol) were successively added, and the resulting mixture was stirred at 90° C. for 6 h. The reaction mixture was cooled to room temperature and filtered and the filtrate was concentrated under reduced pressure. The crude was dissolved in ethyl acetate (50 mL), and water (50 mL) was added for layering and extraction. The organic phase was collected and the aqueous phase was extracted with ethyl acetate (50 mL×2). The organic phases were combined, dried over anhydrous sodium sulfate and filtered and the filtrate was concentrated under reduced pressure and subjected to silica gel column chromatography to afford 3j (564.0 mg, yield: 58.8%).

LC-Ms m/z (ESI): 495.2[M+H]⁺.

### Step 6: Synthesis of 3k

3j (564.0 mg, 1.1 mmol) was dissolved in dichloromethane (10 mL), trifluoroacetic acid (2 mL) was added, and the resulting mixture was stirred at room temperature for 2 h. The reaction system was concentrated under reduced pressure to afford crude 3k.

LC-Ms m/z (ESI): 395.2[M+H]⁺.

### Step 7: Synthesis of 3l-1 and 3l-2

Intermediate 1 (280.0 mg, 1.3 mmol) was dissolved in DMF (10 mL), 3k (490.0 mg, 1.1 mmol), HATU (650.0 mg, 1.7 mmol) and DIPEA (740.0 mg, 5.7 mmol) were successively added, and the mixture was reacted at room temperature for 1 h. Water (50 mL) was added and the resulting mixture was extracted with ethyl acetate (15 mL×3). The organic phases were combined and washed with saturated brine. The organic phase was collected, dried over anhydrous sodium sulfate, filtered and concentrated and the residue was subjected to silica gel column chromatography to afford products 3l-1 (156.0 mg, yield: 22.8%) and 3l-2 (134.0 g, yield: 19.6%).

LC-Ms m/z (ESI): 600.3[M+H]⁺.

### Step 8: Synthesis of 3m-1 and 3m-2

3l-1 (35.0 mg, 0.06 mmol) was dissolved in methanol (3 mL), potassium fluoride (11.0 mg, 0.2 mmol) was added, and the mixture was reacted at room temperature for 18 h. Water (10 mL) was added and the resulting mixture was extracted with ethyl acetate (10 mL×3). The organic phases were combined, dried over anhydrous sodium sulfate, filtered and concentrated to afford crude 3m-1.

LC-Ms m/z (ESI): 528.3[M+H]⁺.

3l-2 (42.0 mg, 0.07 mmol) was dissolved in methanol (3 mL), potassium fluoride (13.0 mg, 0.2 mmol) was added, and the mixture was reacted at room temperature for 18 h. Water (10 mL) was added and the resulting mixture was extracted with ethyl acetate (10 mL×3). The organic phases were combined, dried over anhydrous sodium sulfate, filtered and concentrated to afford crude 3m-2.

LC-Ms m/z (ESI): 528.3[M+H]⁺.

### Step 9: Synthesis of Compound 3-1 and Compound 3-2

3m-1 (31.0 mg, 0.06 mmol) was dissolved in THF (3 mL), a solution of lithium hydroxide monohydrate (74.0 mg, 0.18 mmol) in water (1 mL) was added dropwise, and the resulting mixture was stirred at room temperature for 18 h. Water (6 mL) was added and the resulting mixture was adjusted with 1 N hydrochloric acid aqueous solution to pH=5-6 and extracted with ethyl acetate (15 mL×3). The organic phases were combined, dried over anhydrous sodium sulfate, filtered and concentrated to afford a crude, which was subjected to prep-HPLC to afford the trifluoroacetate of compound 3-1 (8.0 mg, yield: 18.6%).

Conditions for preparative liquid chromatography: instrument and preparative column: waters 2767 (preparative liquid phase chromatographic instrument) was used; the preparative column model was SunFire@Prep C18 (19 mm×250 mm). Mobile phase system: acetonitrile/water (containing 1% TFA).

Trifluoroacetate of compound 3-1: LC-Ms m/z=500.2 [M+1]⁺.

¹H NMR (400 MHz, DMSO-d₆) δ 8.90 (d, 1H), 8.48 (d, 1H), 7.53-7.43 (m, 2H), 7.24-7.17 (m, 1H), 7.16-7.08 (m, 2H), 6.09 (s, 1H), 6.05-5.98 (m, 1H), 5.56-5.46 (m, 1H), 5.28-5.17 (m, 1H), 3.96 (s, 1H), 2.81 (d, 2H), 2.08 (s, 3H), 1.88 (s, 3H), 1.85-1.67 (m, 5H), 1.39-1.27 (m, 1H), 0.90-0.80 (m, 6H).

3m-2 (36.0 mg, 0.07 mmol) was dissolved in THF (3 mL), a solution of lithium hydroxide monohydrate (86.0 mg, 0.20 mmol) in water (1 mL) was added dropwise, and the resulting mixture was stirred at room temperature for 18 h. Water (6 mL) was added and the resulting mixture was adjusted with 1N hydrochloric acid aqueous solution to pH=5-6 and extracted with ethyl acetate (15 mL×3). The organic phases were combined, dried over anhydrous sodium sulfate, filtered and concentrated to afford a crude, which was subjected to prep-HPLC to afford the trifluoroacetate of compound 3-2 (10.0 mg, yield: 20.2%).

Conditions for preparative liquid chromatography:
instrument and preparative column: waters 2767 (preparative liquid phase chromatographic instrument) was used; the preparative column model was SunFire@Prep C18 (19 mm×250 mm). Mobile phase system: acetonitrile/water (containing 1% TFA).

Trifluoroacetate of compound 3-2: MS m/z=500.2 [M+H]⁺.

¹H NMR (400 MHz, DMSO-d₆) δ 9.03 (d, 1H), 8.52 (d, 1H), 7.60-7.50 (m, 2H), 7.26-7.18 (m, 1H), 7.17-7.11 (m, 2H), 6.18 (s, 1H), 6.11-6.04 (m, 1H), 5.62-5.52 (m, 1H), 5.23-5.14 (m, 1H), 3.99 (s, 1H), 2.87-2.73 (m, 2H), 2.11 (s, 3H), 1.91 (s, 6H), 1.72-1.61 (m, 1H), 1.59-1.50 (m, 1H), 1.20-1.09 (m, 1H), 0.80-0.70 (m, 6H).

### Example 4: Preparation of Compound 4 and Stereoisomer Thereof

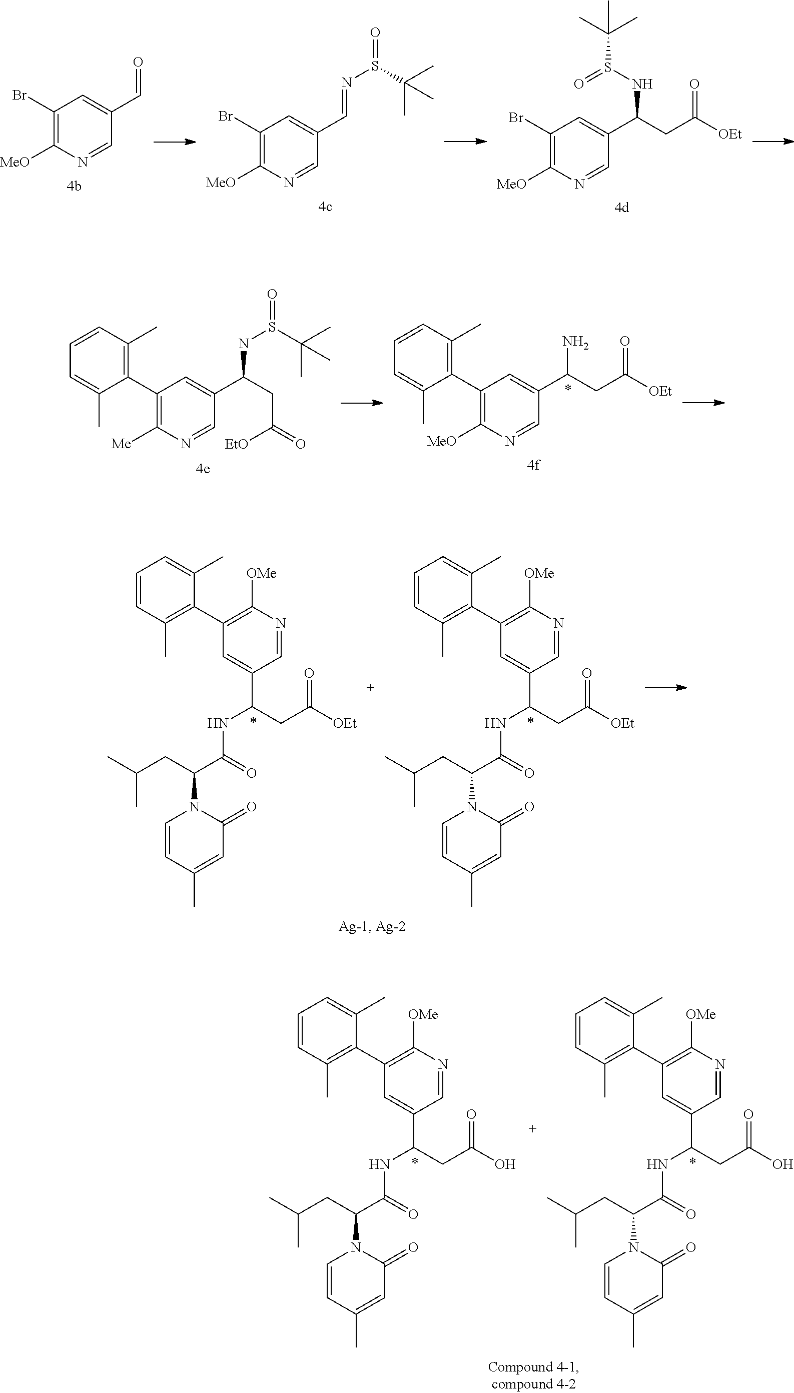

For the trifluoroacetate of compound 4-1 (40 mg) and trifluoroacetate of compound 4-2 (40 mg), reference can be made to example 1 for the reaction conditions and operations.

HPLC preparation conditions: instrument: waters 2767 preparative chromatographic column: SunFire@Prep C18 (19 mm×150 mm); composition of mobile phases: mobile phase A: acetonitrile, and mobile phase B: water (containing 0.1% TFA).

Compound 4-1: Ms m/z (ESI): 506.2 [M+H]⁺.

¹H NMR (400 MHz, CDCl₃) δ 8.11 (d, 1H), 8.06 (d, 1H), 7.40 (d, 1H), 7.24-7.11 (m, 2H), 7.05 (d, 2H), 6.39 (s, 1H), 6.19-6.11 (m, 1H), 5.68-5.59 (m, 1H), 5.36-5.26 (m, 1H), 3.86 (s, 3H), 2.99-2.76 (m, 2H), 2.15 (s, 3H), 2.03-1.93 (m, 1H), 1.90 (s, 3H), 1.82 (s, 3H), 1.81-1.71 (m, 1H), 1.53-1.39 (m, 1H), 0.96-0.87 (m 6H).

Compound 4-2: Ms m/z (ESI): 506.2 [M+H]⁺.

¹H NMR (400 MHz, CDCl₃) δ 8.29 (s, 1H), 8.16 (d, 1H), 7.59 (d, 1H), 7.47 (s, 1H), 7.22-7.14 (m, 1H), 7.13-7.06 (m, 2H), 6.47 (s, 1H), 6.29 (d, 1H), 5.76-5.68 (m, 1H), 5.38-5.28 (m, 1H), 3.93 (s, 3H), 2.94-2.75 (m, 2H), 2.21 (s, 3H), 1.99 (s, 3H), 1.97 (s, 3H), 1.92-1.69 (m, 2H), 1.42-1.24 (m, 1H), 0.90-0.75 (m, 6H).

### Example 5: Preparation of Compound 5 and Stereoisomer Thereof

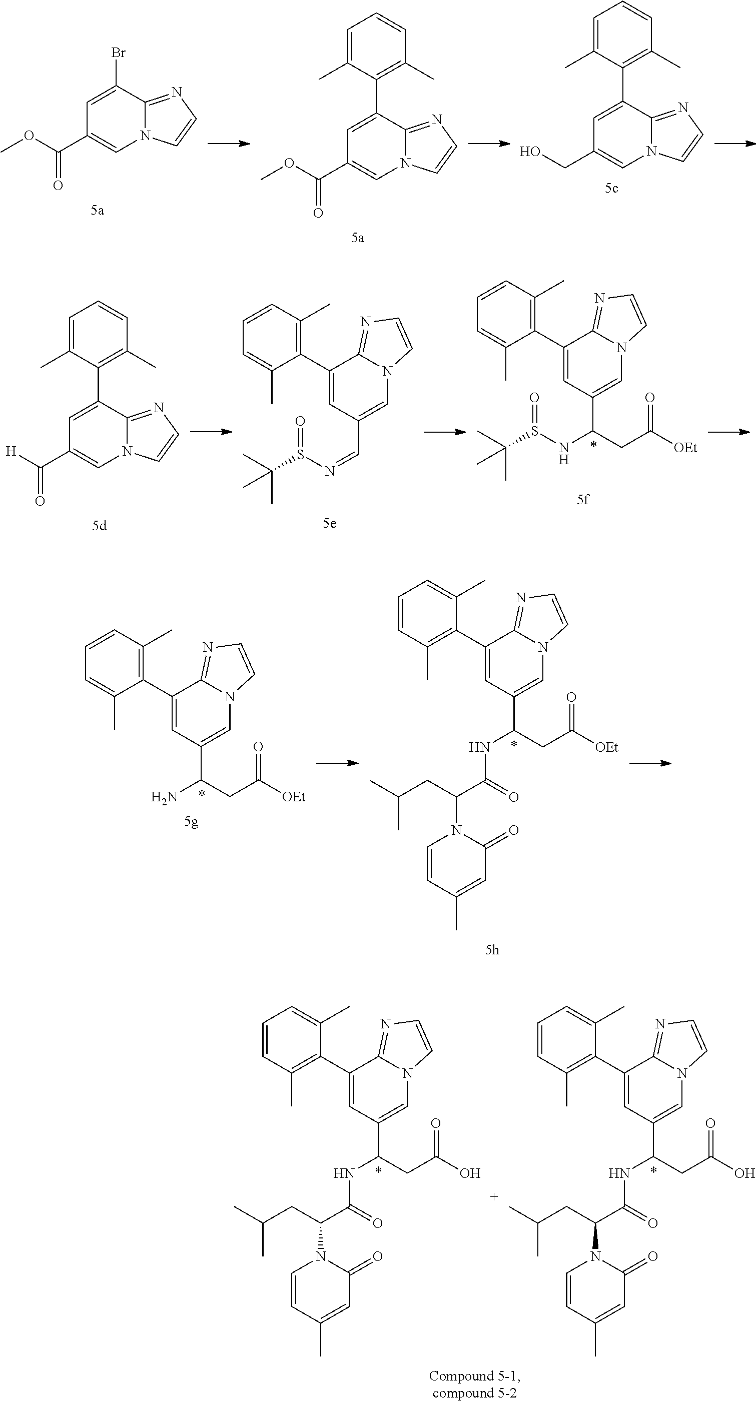

### Step 1: Synthesis of 5b

Under nitrogen protection, 5a (3 g, 12.45 mmol), (2,6-dimethyl phenyl) boronic acid (3.73 g, 24.9 mmol) and cesium carbonate (12.17 g, 37.35 mmol) were added to 1,4-dioxane (48.0 mL) and water (8.0 mL), Pd(PPh₃)₄ (1.44 g, 1.25 mmol) was added, and the resulting mixture was reacted at 100° C. in a sealed tube for 6 h. The reaction mixture was cooled to room temperature and filtered through celite. The solvent was removed under reduced pressure, and the residue was subjected to flash chromatography on a silica gel column (petroleum ether/ethyl acetate 100%-40%) to afford 5b (1.53 g, yield: 44%).

Ms m/z (ESI): 281.2 [M+H]⁺.

### Step 2: Synthesis of 5c

Under nitrogen protection, 5b (1.17 g, 4 mmol) was dissolved in dry THF (17 mL) and at 0° C., lithium aluminum hydride (400 mg, 10.43 mmol) was slowly added. The resulting mixture was reacted at room temperature for 1 h. 10% sodium sulfate aqueous solution (10 mL) was added and the solid was filtered. Ethyl acetate (100 mL) was added and the resulting mixture was washed with water (30 ml) once and washed with saturated brine (30 mL) once, dried over anhydrous sodium sulfate and filtered. The filtrate was concentrated under reduced pressure to afford crude 5c (1 g, 95%).

Ms m/z (ESI): 253.2 [M+H]⁺.

### Step 3: Synthesis of 5d

Under nitrogen protection, 5c (1 g, 3.96 mmol) was dissolved in dry dichloromethane (20 mL), sodium bicarbonate (670 mg, 7.92 mmol) and Dess-Martin periodinane (2.52 g, 5.94 mmol) were added, and the resulting mixture was reacted at room temperature for 5 h. Saturated sodium thiosulfate solution (10 mL) and saturated sodium bicarbonate solution (10 mL) were added and the resulting mixture was extracted with ethyl acetate (50 mL×3), dried over anhydrous sodium sulfate and filtered. The filtrate was concentrated under reduced pressure to afford crude 5d (1 g, 99%).

### Step 4: Synthesis of 5e

Under nitrogen protection, 5d (1 g, 4 mmol) and R-tert-butylsulfinamide (0.73 g, 6 mmol) were dissolved in THF (9 mL), tetraethyl titanate (1.37 g, 6 mmol) was slowly added, and the resulting mixture was reacted at 40° C. for 2 h. Water (50 mL) and ethyl acetate (50 ml) were added and the mixture was filtered through celite and washed with ethyl acetate (100 ml×2). The organic phases were combined, washed with saturated brine (100 mL), dried over anhydrous sodium sulfate and filtered and the filtrate was concentrated under reduced pressure. The residue was subjected to flash column chromatography on a silica gel column (dichloromethane/methanol: 100%-95%) to afford 5e (580 mg, three-step total yield: 40%).

Ms m/z (ESI): 354.2 [M+H]⁺.

### Step 5: Synthesis of 5f

Zinc powder (1.38 g, 21.16 mmol) was added to dry THF (9 mL), the mixture was subjected to nitrogen replacement three times, CuCl (451 mg, 4.56 mmol) was added, and the resulting mixture was reacted at 60° C. for 2 h. The reaction mixture was cooled to room temperature, ethyl bromoacetate (1.25 g, 7.47 mmol) was slowly added, and the resulting mixture was reacted at 60° C. for 1 h and cooled to 0° C. A solution of 5e (480 mg, 7.83 mmol) in THF (1 mL) was added and the mixture was stirred at 0° C. for 3 h. The reaction mixture was filtered through celite and 1 N HCl (50 mL) was added. The resulting mixture was extracted with ethyl acetate (50 ml×3), washed with saturated brine (50 mL), dried over anhydrous sodium sulfate and filtered and the filtrate was concentrated under reduced pressure. The residue was subjected to flash column chromatography (dichloromethane/methanol: 100%-95%) to afford 5f (510 mg, yield: 84.92%).

Ms m/z (ESI): 442.2 [M+H]⁺.

### Steps 6-8

For the trifluoroacetate of compound 5-1 (18 mg, retention time=5.211 min) and trifluoroacetate of compound 5-2 (14 mg, retention time=7.594 min), reference can be made to example 1 for the reaction conditions and operations.

HPLC preparation conditions: instrument: waters 2767 preparative chromatographic column: SunFire@Prep C18 (19 mm×150 mm); composition of mobile phases: mobile phase A: acetonitrile, and mobile phase B: water (containing 0.1% TFA).

Trifluoroacetate of compound 5-1:

¹H NMR (400 MHz, CDCl₃) δ 8.76-8.55 (m, 2H), 7.74-7.63 (m, 1H), 7.61-7.53 (m, 1H), 7.37 (d, 1H), 7.23 (s, 1H), 7.17 (t, 1H), 7.07-7.00 (m, 2H), 6.21 (s, 1H), 6.11 (d, 1H), 5.71-5.61 (m, 1H), 5.46-5.38 (m, 1H), 3.10-2.90 (m, 2H), 2.13 (s, 3H), 2.06-1.94 (m, 1H), 1.92-1.73 (m, 7H), 1.55-1.43 (m, 1H), 0.98-0.86 (m, 6H).

MS m/z (ESI): 515.2[M+H]⁺.

Trifluoroacetate of compound 5-2: MS m/z (ESI): 515.2[M+H]⁺.

### Example 6: Preparation of Compound 6 and Stereoisomer Thereof

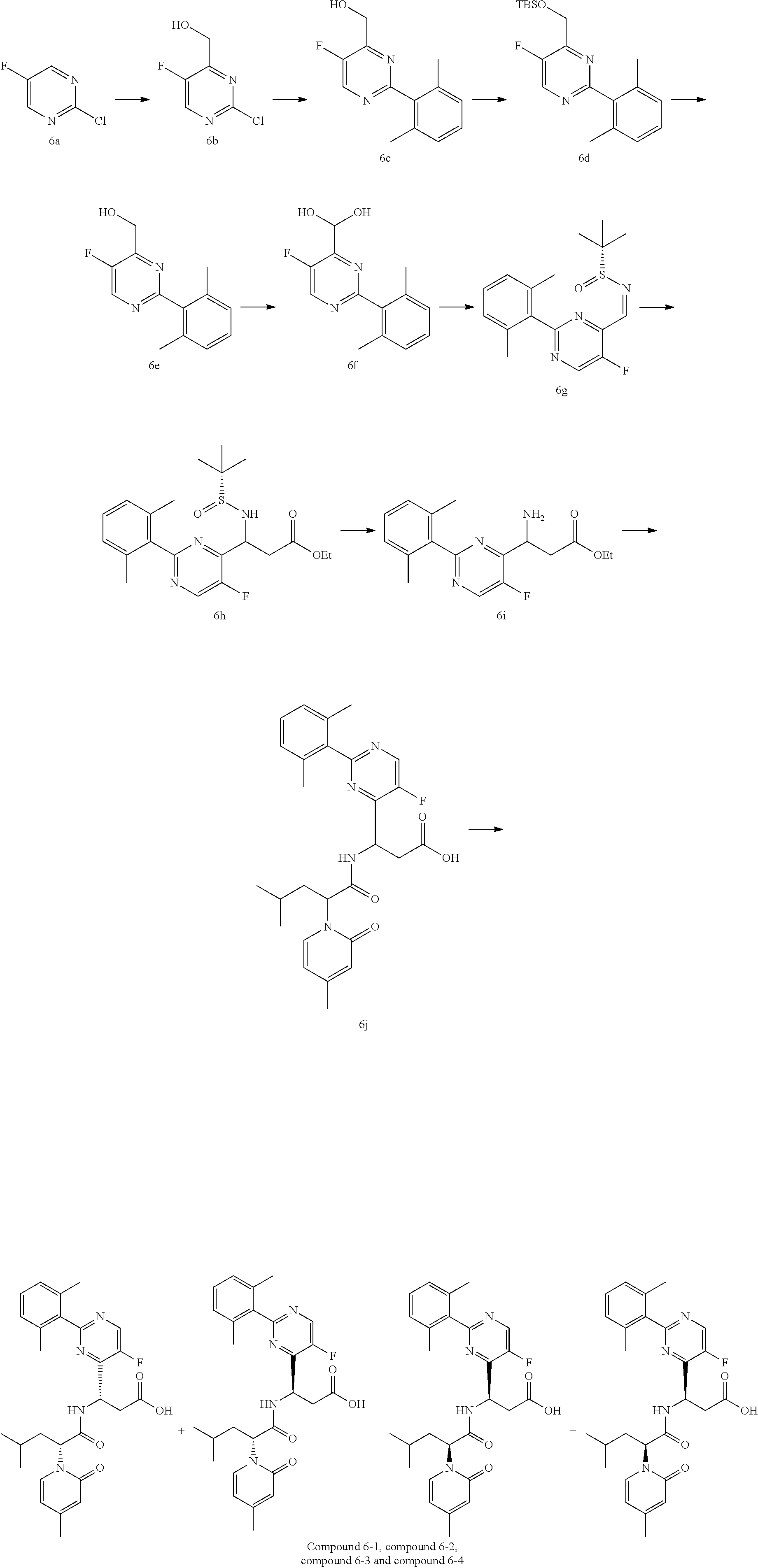

### Step 1: Synthesis of 6b

6a (7.2 g, 54.29 mmol) was dissolved in methanol (500 mL), trifluoroacetic acid (6.87 g, 60.26 mmol) and benzoyl peroxide (15 g, 61.89 mmol) were added, and the resulting mixture was subjected to nitrogen replacement four times, reacted at 65° C. for 24 h and concentrated under reduced pressure to remove the solvent. Saturated sodium bicarbonate (100 mL) was added. The resulting mixture was extracted with dichloromethane (300 mL×2), dried over anhydrous sodium sulfate and filtered and the filtrate was concentrated under reduced pressure. The residue was subjected to silica gel column chromatography to afford 6b (3.3 g, yield: 37.39%).

Ms m/z (ESI): 163.0[M+H]⁺.

### Step 2: Synthesis of 6c

Under nitrogen protection, 6b (3 g, 18.46 mmol), (2,6-dimethylphenyl)boronic acid (5.55 g, 37 mmol), Pd(dppf)Cl₂·DCM (1.5 g, 1.83 mmol) and cesium carbonate (18.4 g, 55.38 mmol) were added to 1,4-dioxane (60.0 mL) and water (10.0 mL), and the mixture was stirred at 100° C. for 3 h. The reaction mixture was cooled to room temperature. The solvent was removed under reduced pressure, water (60 mL) was added, and the resulting mixture was extracted with ethyl acetate (100 mL×3). The organic phases were combined, dried over anhydrous sodium sulfate and filtered and the filtrate was concentrated under reduced pressure. The residue was subjected to flash chromatography on a silica gel column to afford 6c (5.3 g).

Ms m/z (ESI): 233.2 [M+H]⁺.

### Step 3: Synthesis of 6d

Under nitrogen protection, 6c (4.8 g, 15.5 mmol) was dissolved in DMF (35 mL), imidazole (1.58 g, 23.25 mmol) and tert-butyldimethylsilane (2.8 g, 18.6 mmol) were successively added, and the resulting mixture was reacted at room temperature overnight. Ethyl acetate (100 mL) was added and the mixture was washed with water twice (30 mL×2) and washed with saturated brine, dried over anhydrous sodium sulfate and filtered. The filtrate was concentrated under reduced pressure and the residue was subjected to flash chromatography on a silica gel column to afford 6d (2.5 g, 46.55%).

### Step 4: Synthesis of 6e

Under nitrogen protection, 6d (2.3 g, 6.64 mmol) was dissolved in THF (40 mL), acetic acid (710 mg, 11.82 mmol) and TBAF solution (12 mL, 12 mmol) were added, and the resulting mixture was reacted at room temperature for 5 h. Saturated ammonium chloride solution (40 mL) was added and the mixture was extracted with ethyl acetate three times (60 mL×3), washed with saturated brine (40 mL), dried over anhydrous sodium sulfate and filtered. The filtrate was concentrated under reduced pressure and the residue was subjected to flash chromatography on a silica gel column (ethyl acetate/petroleum ether: 0%-20%) to afford 6e (1.4 g, yield: 90.78%).

¹H NMR (400 MHz, CDCl₃) δ 8.65 (d, 1H), 7.28-7.21 (m, 1H), 7.13 (d, 2H), 4.92 (d, 2H), 3.20 (s, 1H), 2.08 (s, 6H).

### Step 5: Synthesis of 6f

Under nitrogen protection, 6e (200 mg, 0.86 mmol) was dissolved in dry dichloromethane (6 mL), sodium bicarbonate (145 mg, 1.72 mmol) and Dess-Martin periodinane (548 mg, 1.29 mmol) were added, and the resulting mixture was reacted at room temperature for 1 h. Saturated sodium thiosulfate solution (8 mL) and saturated sodium bicarbonate solution (8 mL) were added and the resulting mixture was extracted with ethyl acetate (30 mL×3), dried over anhydrous sodium sulfate and filtered. The filtrate was concentrated under reduced pressure to afford crude 6f (1 g, 99%).

Ms m/z (ESI): 249.1 [M+H]⁺.

### Steps 6-10

For the synthesis of 6-1 (13 mg, retention time=1.395 min), compound 6-2 (22 mg, retention time=1.644 min), compound 6-3 (11 mg, retention time=1.406 min), and compound 6-4 (10 mg, retention time=1.596 min), reference can be made to example 1 for the reaction conditions and operations.

SFC resolution conditions: instrument: Waters 150 MGM; chromatographic column: Chiralpak Column; mobile phase: A for CO₂ and B for MeOH; gradient: 30% phase B isocratic elution; flow rate: 110 mL/min.

Ms m/z (ESI): 495.2 [M+H]⁺.

Compound 6-1: ¹H NMR (400 MHz, CDCl₃) δ 8.53 (d, 1H), 7.99 (d, 1H), 7.33 (d, 1H), 7.16 (t, 1H), 6.99 (d, 2H), 6.30 (s, 1H), 5.94-5.89 (m, 1H), 5.76 (t, 1H), 5.67-5.59 (m, 1H), 2.92-2.76 (m, 2H), 2.11 (s, 3H), 1.99-1.91 (m, 1H), 1.90 (s, 6H), 1.79-1.69 (m, 1H), 1.51-1.37 (m, 1H), 0.96-0.88 (m, 6H).

Compound 6-2: ¹H NMR (400 MHz, CDCl₃) δ 8.59 (d, 1H), 8.09 (d, 1H), 7.55 (d, 1H), 7.22-7.17 (m, 1H), 7.10-7.03 (m, 2H), 6.39 (s, 1H), 6.22-6.16 (m, 1H), 5.80-5.74 (m, 1H), 5.68-5.61 (m, 1H), 3.08-2.99 (m, 1H), 2.94-2.85 (m, 1H), 2.16 (s, 3H), 2.05 (s, 6H), 1.84-1.74 (m, 1H), 1.70-1.60 (m, 1H), 1.41-1.28 (m, 1H), 0.75 (d, 3H), 0.70 (d, 3H).

Compound 6-3: ¹H NMR (400 MHz, CDCl₃) δ 8.53 (d, 1H), 7.99 (d, 1H), 7.33 (d, 1H), 7.16 (t, 1H), 6.99 (d, 2H), 6.30 (s, 1H), 5.94-5.89 (m, 1H), 5.76 (t, 1H), 5.67-5.60 (m, 1H), 2.92-2.76 (m, 2H), 2.11 (s, 3H), 2.01-1.91 (m, 1H), 1.90 (s, 6H), 1.79-1.69 (m, 1H), 1.51-1.37 (m, 1H), 0.96-0.88 (m, 6H).

Compound 6-4: ¹H NMR (400 MHz, CDCl₃) δ 8.59 (d, 1H), 8.09 (d, 1H), 7.55 (d, 1H), 7.22-7.17 (m, 1H), 7.10-7.03 (m, 2H), 6.39 (s, 1H), 6.20-6.16 (m, 1H), 5.80-5.74 (m, 1H), 5.68-5.61 (m, 1H), 3.08-2.99 (m, 1H), 2.94-2.85 (m, 1H), 2.16 (s, 3H), 2.05 (s, 6H), 1.84-1.74 (m, 1H), 1.70-1.60 (m, 1H), 1.41-1.28 (m, 1H), 0.75 (d, 3H), 0.70 (d, 3H).

### Example 7: Preparation of Compound 7 and Stereoisomer Thereof

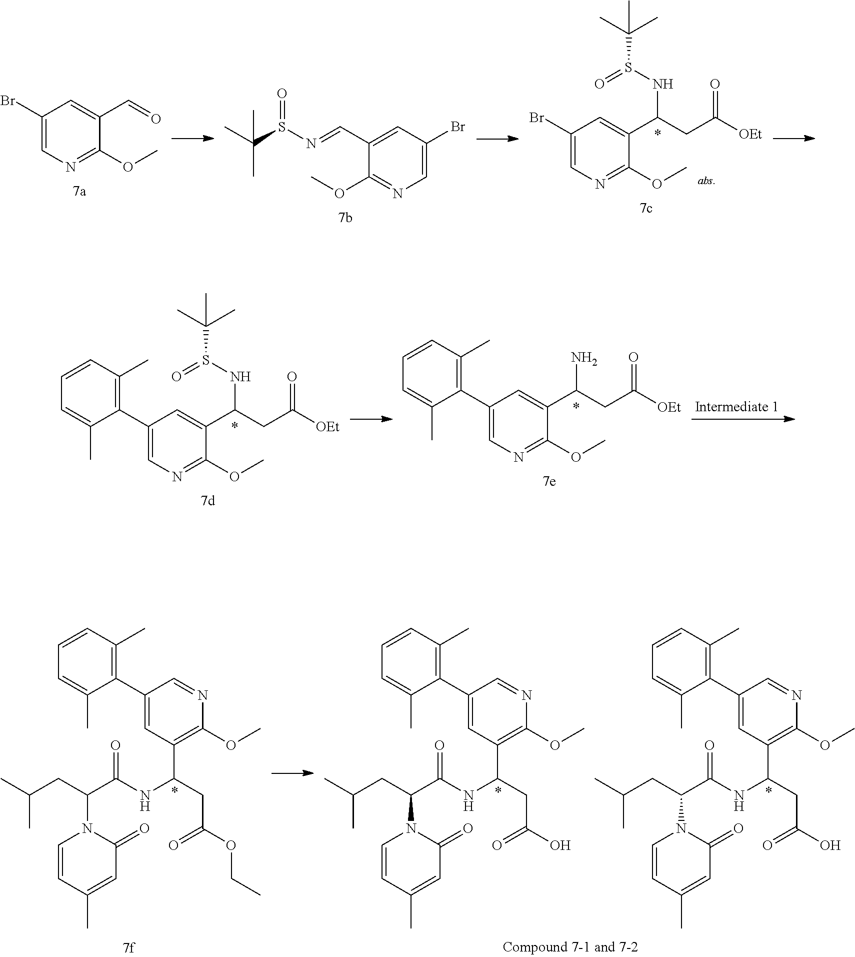

For the trifluoroacetate of compound 7-1 (12 mg, retention time=1.381 min) and trifluoroacetate of compound 7-2 (11 mg, retention time=1.612 min), reference can be made to example 6 for the reaction conditions and operations.

HPLC preparation conditions: instrument: waters 2767 preparative chromatographic column: SunFire@Prep C18 (19 mm×150 mm); composition of mobile phases: mobile phase A: acetonitrile, and mobile phase B: water (containing 0.1% TFA).

Trifluoroacetate of compound 7-1: Ms m/z (ESI): 506.2[M+H]⁺.

¹H NMR (400 MHz, DMSO-d₆) δ 8.76 (d, 1H), 7.81 (d, 1H), 7.47 (d, 1H), 7.32 (d, 1H), 7.22-7.15 (m, 1H), 7.14-7.07 (m, 2H), 6.03 (s, 1H), 5.98 (dd, 1H), 5.57-5.49 (m, 1H), 5.40-5.31 (m, 1H), 3.92 (s, 3H), 2.76-2.67 (m, 1H), 2.63-2.53 (m, 1H), 2.05 (s, 3H), 1.96 (s, 3H), 1.82-1.66 (m, 5H), 1.39-1.26 (m, 1H), 0.86 (d, 3H), 0.83 (d, 3H).

Trifluoroacetate of compound 7-2: Ms m/z (ESI): 506.2[M+H]⁺.

¹H NMR (400 MHz, DMSO-d₆) δ 8.92 (d, 1H), 7.87 (d, 1H), 7.56 (d, 1H), 7.32 (d, 1H), 7.22-7.11 (m, 3H), 6.17 (s, 1H), 6.08 (dd, 1H), 5.64-5.56 (m, 1H), 5.44-5.36 (m, 1H), 3.95 (s, 3H), 2.73-2.64 (m, 1H), 2.62-2.53 (m, 1H), 2.11 (s, 3H), 2.00-1.94 (m, 6H), 1.65-1.57 (m, 2H), 1.21-1.11 (m, 1H), 0.77-0.68 (m, 6H).

Ms m/z (ESI): 506.2[M+H]⁺.

### Example 8: Preparation of Compound 8 and Stereoisomer Thereof

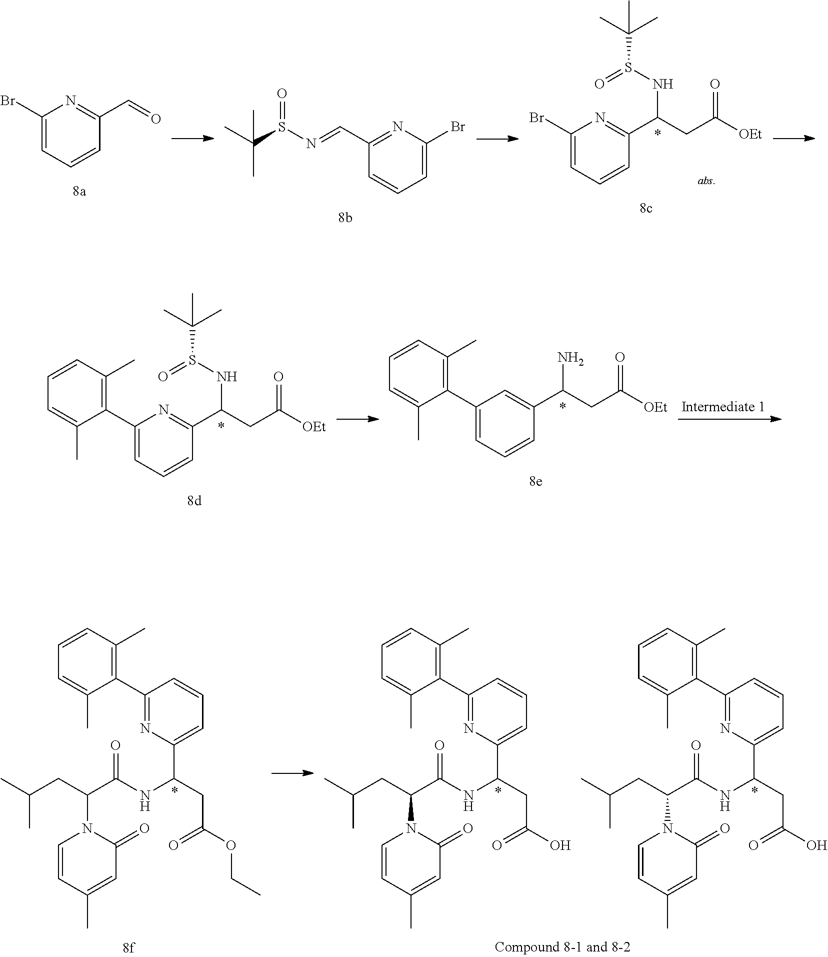

For compound 8-1 (50 mg, retention time: 1.473 min) and compound 8-2 (30 mg, retention time: 1.591 min), reference can be made to example 1 for the reaction conditions and operations;

SFC resolution conditions: instrument: Waters 150 MGM; chromatographic column: Chiralpak Column; composition of mobile phases: mobile phase A: CO2, mobile phase B: IPA (0.1% NH₃·H₂O).

Ammonium salt of compound 8-1:

Ms m/z (ESI): 476.2[M+H]⁺.

¹H NMR (400 MHz, DMSO-d6) δ 9.48 (d, 1H), 7.76 (t, 1H), 7.53 (d, 1H), 7.27 (d, 1H), 7.22-7.14 (m, 1H), 7.13-7.04 (m, 3H), 6.18 (s, 1H), 6.06 (dd, 1H), 5.64-5.50 (m, 1H), 5.09-5.00 (m, 1H), 2.45-2.31 (m, 2H), 2.11 (s, 3H), 1.96 (s, 6H), 1.87-1.68 (m, 2H), 1.28-1.17 (m, 1H), 0.80 (d, 6H).

Ammonium salt of compound 8-2:

Ms m/z (ESI): 476.2[M+H]⁺.

¹H NMR (400 MHz, DMSO-d6) δ 8.94 (d, 1H), 7.80 (t, 1H), 7.56 (d, 1H), 7.27-7.06 (m, 5H), 6.17 (s, 1H), 6.06 (dd, 1H), 5.65-5.57 (m, 1H), 5.31-5.20 (m, 1H), 2.96-2.85 (m, 1H), 2.75-2.63 (m, 1H), 2.11 (s, 3H), 1.97-1.80 (s, 7H), 1.79-1.68 (m, 2H), 1.37-1.24 (m, 1H), 0.91-0.79 (m, 6H).

### Example 9: Preparation of Compound 9

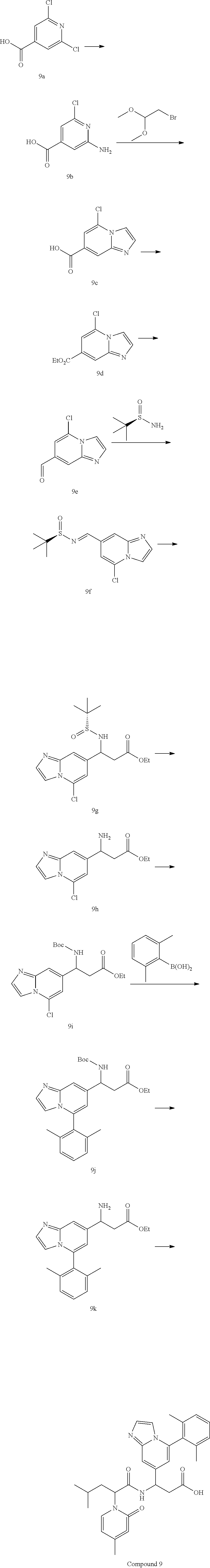

### Step 1: Synthesis of 9b

2,6-dichloroisonicotinic acid (3.0 g, 15.63 mmol) and ammonia water (12 mL) were added to a 30-mL microwave tube, and the mixture was reacted at 165° C. for 3 h. The reaction mixture was concentrated under reduced pressure and the residue was purified by silica gel column chromatography to afford 9b (1.9 g, yield: 70%).

LCMS m/z=173.1 [M+1]⁺.

### Step 2: Synthesis of 9c

9b (1.90 g, 11.01 mmol) and 2-bromo-1,1-dimethoxyethane (2.33 g, 13.76 mmol) were dissolved in ethanol solution (75 mL) and the mixture was subjected to nitrogen replacement three times. At room temperature, hydrogen bromide (1.86 g, 11.01 mmol, 48%) was added dropwise. After the dropwise addition was completed, the mixture system was reacted at 100° C. for 3 h. The reaction system was cooled to room temperature, then cooled at −78° C. for 10 min, and suction-filtered with a sand core funnel to afford 9b (1.77 g, yield: 82%).

LCMS m/z=197.0 [M+1]⁺.

### Step 3: Synthesis of 9d

9c (1.5 g, 7.63 mmol), 4-dimethylaminopyridine (93 mg, 0.763 mmol) and ethanol solution (1.41 g, 30.52 mmol) were dissolved in THF solution (15 mL), 1-(3-dimethylaminopropyl)-3-ethylcarbodiimide hydrochloride (1.61 g, 8.39 mmol) was added in portions, and the resulting mixture was reacted at room temperature for 16 h. The reaction mixture was extracted with ethyl acetate solution (100 mL) and the organic phase was dried over anhydrous sodium sulfate and concentrated under reduced pressure. The residue was purified by silica gel column chromatography to afford 9c (904 mg, yield: 53%).

LCMS m/z=225.1 [M+1]⁺.

### Step 4: Synthesis of 9e

9d (904 mg, 4.02 mmol) was dissolved in dichloromethane solution (10 mL). At −78° C., diisobutylaluminum hydride solution (5.4 mL, 8.04 mmol, 1.5 M) was added dropwise and the mixture was reacted at −78° C. for 1 h. Methanol solution (10 mL) and hydrochloric acid solution (10 mL, 1M) were added, the mixture was extracted with dichloromethane solution (50 mL), the organic phase was dried over anhydrous sodium sulfate and filtered. The filtrate was concentrated under reduced pressure and the residue was purified by silica gel column chromatography to afford 9e (415 mg, yield: 51%).

LCMS m/z=181.1 [M+1]⁺.

### Steps 5-11

Reference can be made to example 1 for the reaction conditions and operations to afford the trifluoroacetate of compound 9 (14 mg).

HPLC preparation conditions: instrument: waters 2767 (preparative liquid phase chromatographic instrument); chromatographic column: XBridge@Prep C18 (30 mm×150 mm); composition of mobile phases: mobile phase A: acetonitrile, mobile phase B: water (containing 0.1% trifluoroacetic acid)

LCMS m/z=515.3 [M+H]⁺.

### Example 10: Preparation of Compound 10 and Stereoisomer Thereof

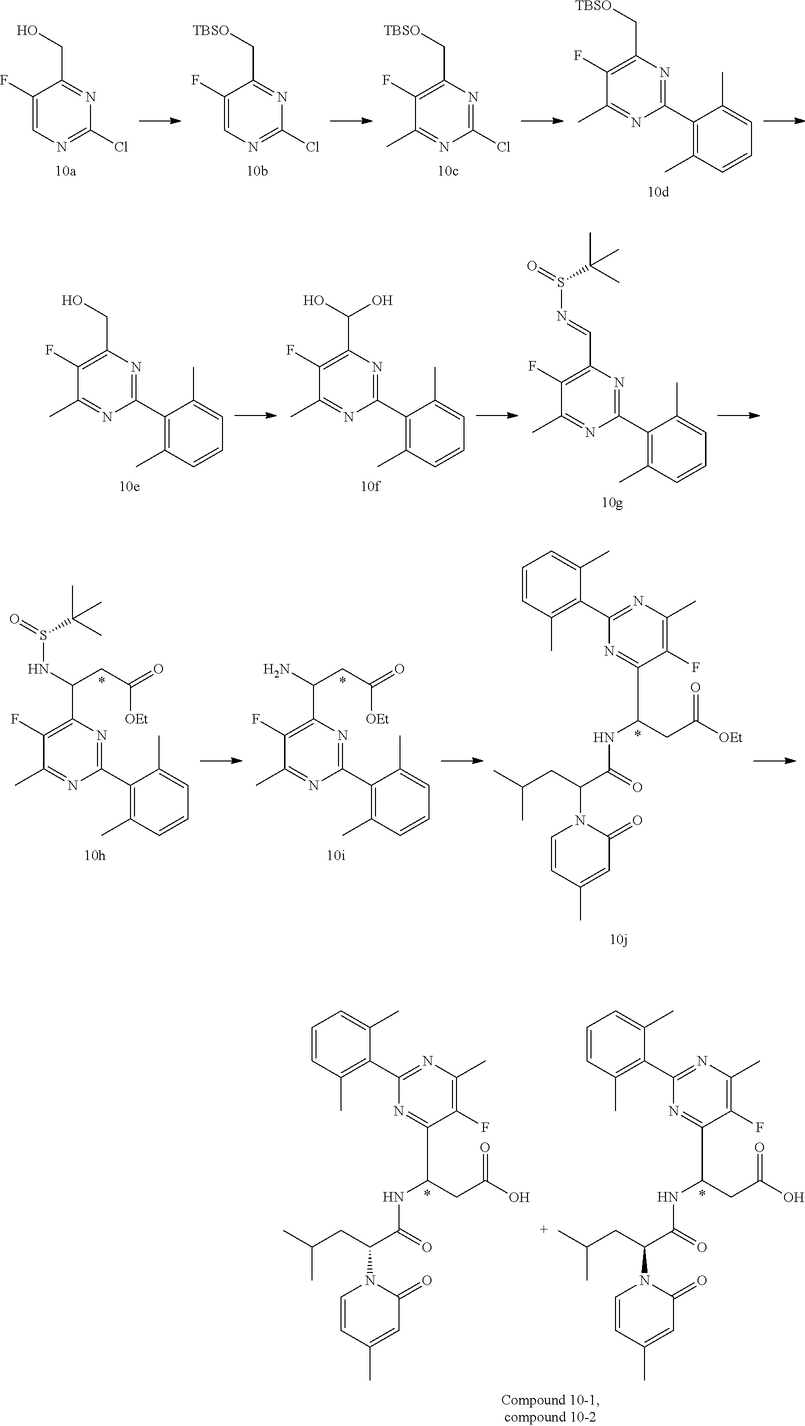

### Step 1: Synthesis of 10b

Under nitrogen protection, 10a (3.4 g, 20.92 mmol) was dissolved in DMF (50 mL), imidazole (2.14 g, 31.38 mmol) and tert-butyldimethylsilane (3.78 g, 25.10 mmol) were successively added, and the resulting mixture was reacted at room temperature overnight. Ethyl acetate (100 mL) was added and the mixture was washed with water (30 mL×2) and washed with saturated brine, dried over anhydrous sodium sulfate and filtered. The filtrate was concentrated under reduced pressure and the residue was subjected to flash chromatography on a silica gel column (ethyl acetate/petroleum ether: 0%-20%) to afford 10b (5.3 g, 91.52%).

MS m/z (ESI): 277.1 [M+H]⁺.

### Step 2: Synthesis of 10c

Under nitrogen protection, 10b (4.1 g, 14.81 mmol) was dissolved in diethyl ether (46 mL). At −30° C., methyllithium solution (7.7 mL, 21.98 mmol, 2 M in THF) was slowly added and the resulting mixture was reacted at −30° C. for 30 min and reacted at 0° C. for 30 min. At 0° C., a solution of acetic acid (0.92 g, 15.4 mmol) and water (0.15 g, 8.29 mmol) in THF (2 mL) was added and the mixture was reacted at room temperature for 5 min. A solution of 2,3-dichloro-5,6-dicyanobenzoquinone (3.5 g, 15.40 mmol) in THF (17 mL) was added and the resulting mixture was stirred at room temperature for 20 min. At 0° C., 3 M sodium hydroxide solution (11 mL) was added and stirred for 5 min. Ethyl acetate (80 mL) was added and the resulting mixture was filtered through celite and concentrated under reduced pressure. The residue was subjected to flash chromatography on a silica gel column (ethyl acetate/petroleum ether: 0%-15%) to afford 10c (2.44 g, 56.65%).

MS m/z (ESI): 291.1 [M+H]⁺.

### Step 3: Synthesis of 10d

Under nitrogen protection, 10c (1.5 g, 5.16 mmol), (2,6-dimethylphenyl)boronic acid (1.55 g, 10.32 mmol), Pd(dppf)Cl₂·DCM (0.42 g, 0.51 mmol) and cesium carbonate (5.04 g, 15.48 mmol) were added to 1,4-dioxane (17.0 mL) and water (3.5 mL), and the mixture was stirred at 100° C. for 3 h. The reaction mixture was cooled to room temperature. The solvent was removed under reduced pressure, water (30 mL) was added, and the resulting mixture was extracted with ethyl acetate (60 mL×3). The organic phases were combined, dried over anhydrous sodium sulfate and filtered and the filtrate was concentrated under reduced pressure. The residue was subjected to flash chromatography on a silica gel column (ethyl acetate/petroleum ether: 0%-20%) to afford 10d (1.8 g, 96.75%).

MS m/z (ESI): 361.2 [M+H]⁺.

### Step 4: Synthesis of 10e

Under nitrogen protection, 10d (1.8 g, 4.99 mmol) was dissolved in THF (30 mL), acetic acid (0.51 mL, 8.88 mmol) and TBAF solution (9 mL, 8.98 mmol, 1 M in THF) were added, and the resulting mixture was reacted at room temperature for 5 h. Saturated ammonium chloride solution (20 mL) was added and the mixture was extracted with ethyl acetate (30 mL×3), washed with saturated brine (20 mL), dried over anhydrous sodium sulfate and filtered. The filtrate was concentrated under reduced pressure and the residue was subjected to flash chromatography on a silica gel column (ethyl acetate/petroleum ether: 0%-30%) to afford 10e (1.16 g, yield: 94.39%).

MS m/z (ESI): 247.2 [M+H]⁺.

### Steps 5-10

For compounds 10-1 (20 mg, retention time: 2.531 min) and 10-2 (21 mg, retention time: 3.015 min), reference can be made to example 6 for the reaction conditions and operations.

HPLC preparation conditions: instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid).

Compound 10-1: Ms m/z (ESI): 509.3 [M+H]⁺.

H NMR (400 MHz, CDCl₃) δ 7.94 (s, 1H), 7.36 (s, 1H), 7.19-7.12 (m, 1H), 7.05-6.98 (m, 2H), 6.25 (s, 1H), 5.89 (s, 1H), 5.73 (s, 1H), 5.62 (s, 1H), 2.99-2.71 (m, 2H), 2.51 (s, 3H), 2.09 (s, 3H), 1.93 (s, 6H), 1.79-1.68 (m, 1H), 1.47-1.35 (m, 1H), 1.33-1.26 (m, 1H), 0.92-0.78 (m, 6H).

Compound 10-2: Ms m/z (ESI): 509.3 [M+H]⁺.

¹H NMR (400 MHz, CDCl₃) δ 8.00 (d, 1H), 7.53 (d, 1H), 7.20-7.14 (m, 1H), 7.08-7.02 (m, 2H), 6.43 (s, 1H), 6.22-6.16 (m, 1H), 5.71 (t, 1H), 5.61 (q, 1H), 3.02-2.84 (m, 2H), 2.57 (d, 3H), 2.16 (s, 3H), 2.04 (s, 6H), 1.89-1.78 (m, 1H), 1.71-1.61 (m, 1H), 1.41-1.32 (m, 1H), 0.81-0.72 (m, 6H).

### Example 11: Preparation of Compound 11

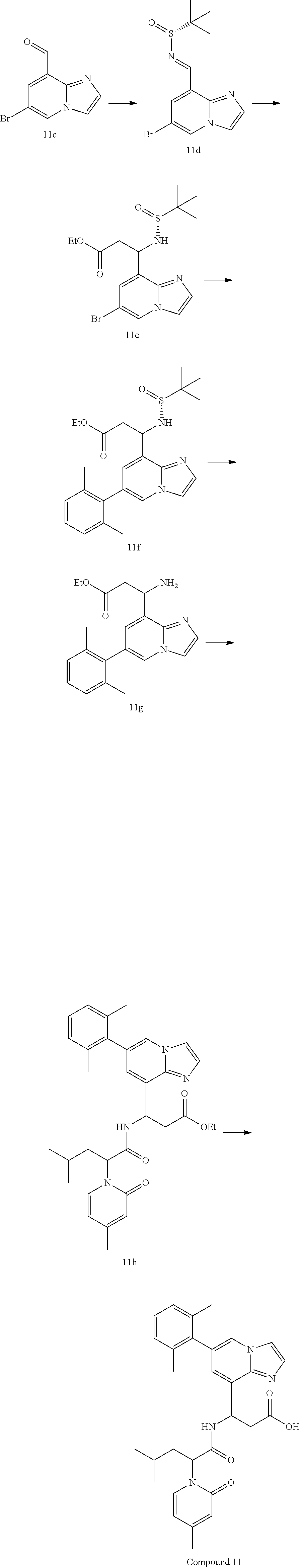

For compound 11, reference can be made to example 5 for the reaction conditions and operations, and the final product was purified by thin layer chromatography (methanol/dichloromethane: 1/15) to afford compound 11 (31 mg).

Ms m/z (ESI): 515.3 [M+H]⁺.

¹H NMR (400 MHz, CDCl₃) δ 8.43 (s, 1H), 7.76 (s, 1H), 7.60 (s, 1H), 7.52 (s, 1H), 7.27-7.21 (m, 1H), 7.19-7.12 (m, 1H), 7.10-6.99 (m, 2H), 6.68 (s, 1H), 6.14 (s, 1H), 5.93-5.75 (m, 2H), 5.67 (s, 1H), 3.18-2.88 (m, 2H), 2.11-2.02 (m, 1H), 2.02-1.93 (m, 6H), 1.90-1.83 (m, 3H), 1.84-1.68 (m, 1H), 1.37-1.28 (m, 1H), 0.91-0.77 (m, 6H).

### Example 12: Preparation of Compound 12 and Stereoisomer Thereof

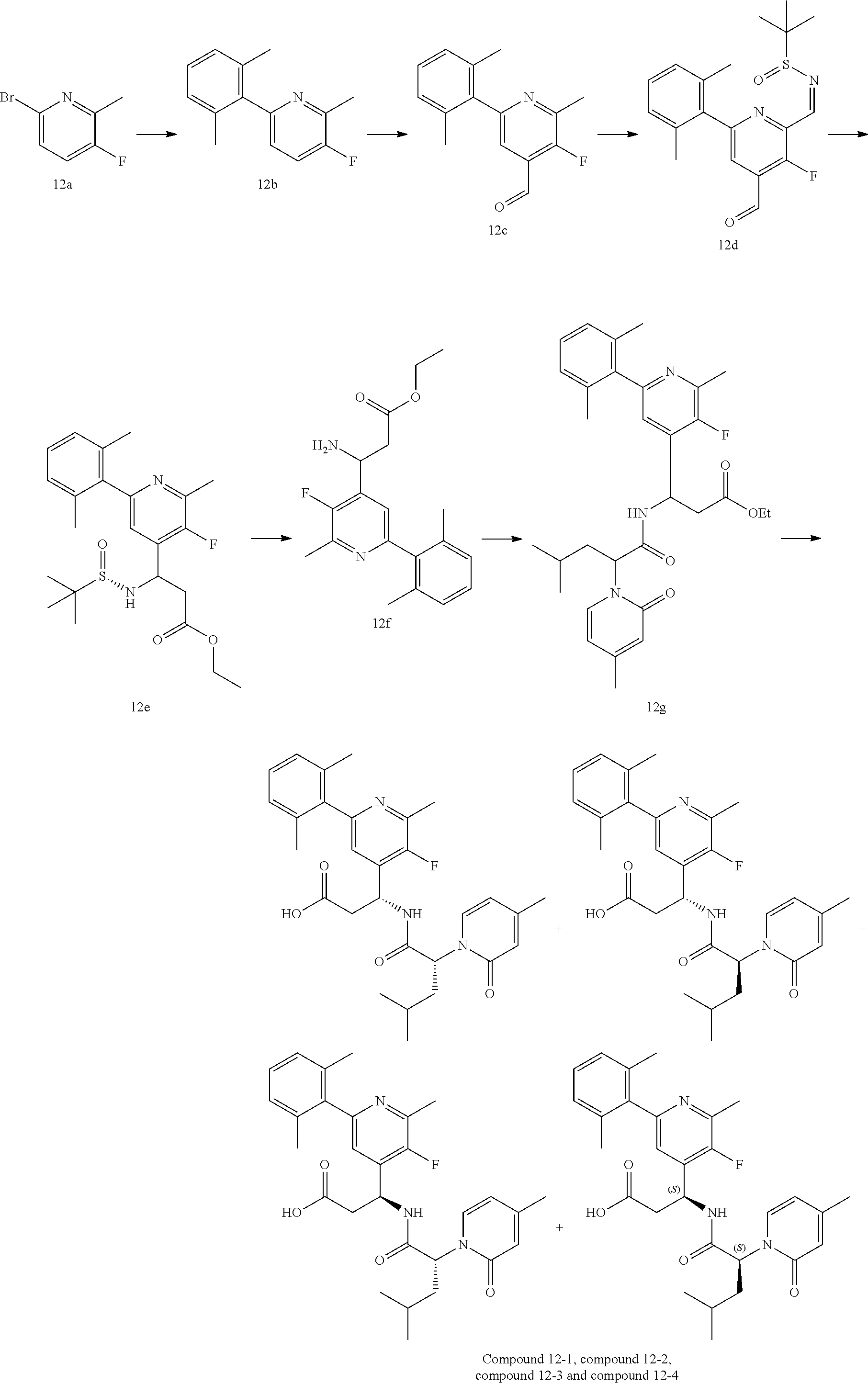

### Step 1: Synthesis of 12b

Under nitrogen protection, 12a (5 g, 26.31 mmol), (2,6-dimethylphenyl)boronic acid (4.74 g, 31.57 mmol), Pd(dppf)Cl₂·DCM (2.15 g, 2.63 mmol) and cesium carbonate (10.91 g, 78.93 mmol) were added to 1,4-dioxane (30.0 mL) and water (6.0 mL), and the mixture was stirred at 100° C. for 3 h. The reaction mixture was cooled to room temperature. The solvent was removed under reduced pressure, water (60 mL) was added, and the resulting mixture was extracted with ethyl acetate (100 mL×3). The organic phases were combined, dried over anhydrous sodium sulfate and filtered and the filtrate was concentrated under reduced pressure. The residue was subjected to flash chromatography on a silica gel column to afford 12b (3.6 g, 63.56%).

Ms m/z (ESI): 216.2[M+H]⁺.

### Step 2: Synthesis of 12c

Under nitrogen protection, 12b (1 g, 4.65 mmol) was dissolved in THF (20 mL). At −78° C., n-butyllithium (0.36 g, 5.58 mmol) was added and stirred for 1 h. DMF (0.68 g, 9.3 mmol) was added and the resulting mixture was reacted at room temperature for 1 h. Ethyl acetate (100 mL) was added and the mixture was washed with water (30 mL×2) and washed with saturated brine, dried over anhydrous sodium sulfate and filtered. The filtrate was concentrated under reduced pressure and the residue was subjected to flash chromatography on a silica gel column to afford 12c (0.61 g, 53.92%).

Ms m/z (ESI): 244.1 [M+H]⁺.

### Steps 3-7

For the ammonium salt of compound 12-1 (8 mg, retention time=0.977 min), ammonium salt of compound 12-2 (21.7 mg, retention time=1.079 min), ammonium salt of compound 12-3 (3 mg, retention time=1.084 min), and ammonium salt of compound 12-4 (28.9 mg, retention time=1.526 min), reference can be made to example 5 for the reaction conditions and operations.

SFC resolution conditions: instrument: Waters 150 MGM; chromatographic column: Chiralpak Column; mobile phase: A for CO₂ and B for MeOH (0.1% NH₃·H₂O); gradient: 20% phase B, isocratic elution; flow rate: 100 mL/min.

Ammonium salt of compound 12-1: Ms m/z (ESI): 508.2 [M+H]⁺.

¹H NMR (400 MHz, DMSO-d₆) δ 9.04 (s, 1H), 7.43 (d, 1H), 7.21-7.14 (m, 1H), 7.10-7.03 (m, 2H), 6.94 (d, 1H), 6.05 (s, 1H), 6.00-5.94 (m, 1H), 5.55-5.48 (m, 1H), 5.41-5.30 (m, 1H), 2.55-2.52 (m, 2H), 2.42 (d, 3H), 2.04 (s, 3H), 1.91-1.76 (m, 7H), 1.75-1.64 (m, 1H), 1.32-1.25 (m, 1H), 0.89-0.77 (m, 6H).

Ammonium salt of compound 12-2: Ms m/z (ESI): 508.2 [M+H]⁺.

¹H NMR (400 MHz, DMSO-d₆) δ 9.04 (s, 1H), 7.43 (d, 1H), 7.21-7.14 (m, 1H), 7.10-7.03 (m, 2H), 6.94 (d, 1H), 6.05 (s, 1H), 6.00-5.94 (m, 1H), 5.55-5.48 (m, 1H), 5.41-5.30 (m, 1H), 2.55-2.52 (m, 2H), 2.42 (d, 3H), 2.04 (s, 3H), 1.91-1.76 (m, 7H), 1.75-1.64 (m, 1H), 1.32-1.25 (m, 1H), 0.89-0.77 (m, 6H).

Ammonium salt of compound 12-3: Ms m/z (ESI): 508.2 [M+H]⁺.

¹H NMR (400 MHz, DMSO-d₆) δ 9.23 (d, 1H), 7.55 (d, 1H), 7.23-7.15 (m, 1H), 7.13-7.06 (m, 3H), 6.20-6.14 (m, 1H), 6.11-6.03 (m 1H), 5.62-5.53 (m, 1H), 5.45-5.36 (m 1H), 2.65-2.56 (m, 2H), 2.46 (d, 3H), 2.11 (s, 3H), 1.95 (s, 6H), 1.71-1.49 (m, 2H), 1.20-1.07 (m, 1H), 0.78-0.68 (m, 6H).

Ammonium salt of compound 12-4: Ms m/z (ESI): 508.2 [M+H]⁺.

¹H NMR (400 MHz, DMSO-d₆) δ 9.23 (d, 1H), 7.55 (d, 1H), 7.23-7.15 (m, 1H), 7.13-7.06 (m, 3H), 6.20-6.14 (m, 1H), 6.11-6.03 (m 1H), 5.62-5.53 (m, 1H), 5.45-5.36 (m 1H), 2.65-2.56 (m, 2H), 2.46 (d, 3H), 2.11 (s, 3H), 1.95 (s, 6H), 1.71-1.49 (m, 2H), 1.20-1.07 (m, 1H), 0.78-0.68 (m, 6H).

### Example 13: Preparation of Compound 13

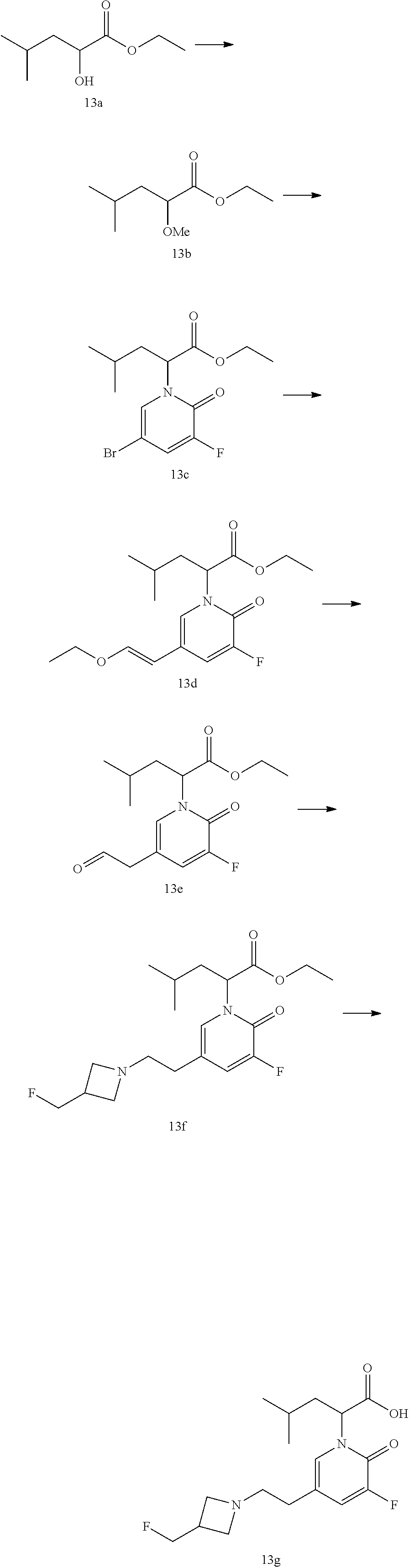

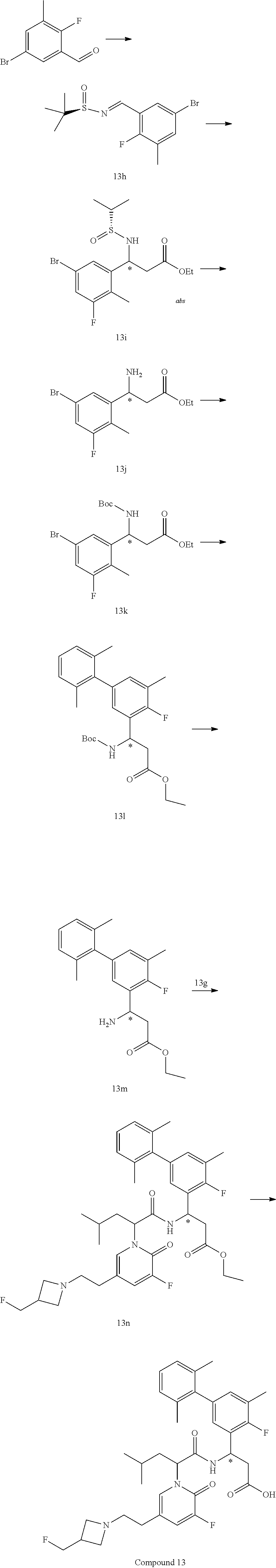

### Step 1: Synthesis of 13b

13a (15 g, 93.68 mmol) was dissolved in dichloromethane (60 mL) and triethylamine (18.96 g, 187.37 mmol) was added. At 0° C., methylsulfonyl chloride (16.09 g, 140.52 mmol) was slowly added dropwise and the resulting mixture was slowly warmed to room temperature and reacted at room temperature for 3 h. Water (60 mL) was added to the reaction system and liquid separation was carried out. The organic phase was washed with saturated sodium chloride solution (50 mL), dried over anhydrous sodium sulfate, filtered and concentrated under reduced pressure and the residue was separated and purified by column chromatography on a silica gel chromatographic column to afford 13b (17.5 g, yield: 78.47%).

Ms m/z (ESI): 239.1 [M+H]⁺.

### Step 2: Synthesis of 13c

3-fluoro-5-bromo-2-pyridone (5.5 g, 28.65 mmol) and 13b (10.23 g, 42.97 mmol) were dissolved in acetonitrile (300 mL), potassium carbonate (7.92 g, 57.3 mmol) was added, and the resulting mixture was reacted at 80° C. for 16 h. The reaction system was cooled to room temperature, concentrated under reduced pressure and separated and purified by column chromatography on a silica gel chromatographic column to afford 13c (8.4 g, yield: 87.74%).

Ms m/z (ESI): 334.0 [M+H]⁺.

### Step 3: Synthesis of 13d

13c (5 g, 14.96 mmol) was dissolved in 1,4-dioxane (120 mL) and water (10 mL), (E)-1-ethoxyethenyl-2-boronic acid pinacol ester (4.44 g, 22.44 mmol), Pd(PPh₃)₄ (1.73 g, 1.5 mmol) and potassium carbonate (4.14 g, 29.92 mmol) were added, and the resulting mixture was reacted at 70° C. under nitrogen protection for 16 h. The reaction system was cooled to room temperature, ethyl acetate (120 mL) was slowly added, and the resulting mixture was washed with water (100 mL×2) and washed with saturated brine (100 mL). The organic phase was dried over anhydrous sodium sulfate, filtered and concentrated under reduced pressure. The residue was subjected to column chromatography on a silica gel chromatographic column (petroleum ether:ethyl acetate=10:1-3:1) to afford 13d (2.4 g, yield: 49.30%).

Ms m/z (ESI): 326.1.0 [M+H]⁺.

### Step 4: Synthesis of 13e

13d (1.95 g, 5.99 mmol) was dissolved in dichloromethane (10 mL), trifluoroacetic acid (4 mL) was added, and the resulting mixture was reacted at 40° C. for 4 h. The reaction system was cooled to room temperature and concentrated under reduced pressure. Saturated sodium bicarbonate aqueous solution (50 mL) was added and the mixture was extracted with ethyl acetate (40 mL×2).

The organic phases were combined, dried over anhydrous sodium sulfate, filtered and concentrated under reduced pressure to afford 13e (1.75 g, 98.82%).

Ms m/z (ESI): 298.1 [M+H]⁺.

### Step 5: Synthesis of 13f

13e (0.5 g, 1.68 mmol) and 3-(fluoromethyl)azetidine hydrochloride (0.32 g, 2.55 mmol) were dissolved in DCE (10 mL) and the mixture was reacted at room temperature for 20 min. Sodium triacetoxyborohydride (0.71 g, 3.34 mmol) was added in portions and the resulting mixture was reacted at room temperature for 16 h. The reaction mixture was concentrated under reduced pressure and then subjected to column chromatography on a silica gel chromatographic column (dichloromethane:methanol=50:1-10:1) to afford 13f (0.42 g, yield: 67.49%).

Ms m/z (ESI): 371.2 [M+H]⁺.

### Step 6: Synthesis of 13g

13f (0.42 g, 1.13 mmol) was dissolved in anhydrous ethanol (6 mL) and water (1 mL), lithium hydroxide (0.095 g, 2.26 mmol) was added, and the resulting mixture was reacted at room temperature for 4 h. The reaction mixture was concentrated under reduced pressure to remove the solvent, adjusted with 1 N hydrochloric acid to pH 7, and purified by column chromatography on a C18 reverse phase column (acetonitrile/water-trifluoroacetic acid) to afford 13g (0.35 g, yield: 90.47%).

Ms m/z (ESI): 343.1 [M+H]⁺.

### Step 7: Synthesis of 13h

5-bromo-2-fluoro-3-methyl benzaldehyde (5 g, 23.03 mmol) and (R)-(+)-tert-butylsulfinamide (3.35 g, 27.64 mmol) were dissolved in dry THF (40 mL). Under nitrogen protection, tetraethyl titanate (7.92 g, 34.73 mmol) was added at room temperature and the resulting mixture was reacted at 40° C. for 3 h. The reaction system was cooled to room temperature and water (40 mL) and ethyl acetate (40 mL) were added. The resulting mixture was stirred at room temperature for 10 min and filtered and the filter cake was washed with ethyl acetate (40 mL×2). The filtrate was subjected to liquid separation, the ethyl acetate layer was washed with water (40 mL×2), and washed with saturated NaCl aqueous solution (40 mL). The organic phase was dried over anhydrous sodium sulfate, filtered and concentrated under reduced pressure to afford 13h (5.8 g, 78.59%).

Ms m/z (ESI): 320.0 [M+H]⁺.

### Steps 8-14

Reference can be made to example 1 for the reaction conditions and operations to afford compound 13 (20 mg).

Ms m/z (ESI): 626.3 [M+H]⁺.

¹H NMR (400 MHz, CD₃OD) δ 7.43-7.31 (m, 2H), 7.14-7.02 (m, 3H), 6.94-6.81 (m, 2H), 5.70-5.45 (m, 2H), 4.60-4.41 (m, 2H), 4.31-3.84 (m, 4H), 3.42-3.32 (m, 2H), 3.26-3.03 (m, 1H), 2.78-2.50 (m, 4H), 2.34-2.243 (m, 3H), 2.00-1.89 (m, 8H), 1.49-1.34 (m, 1H), 0.97-0.87 (m, 6H).

### Example 14: Preparation of Compound 14

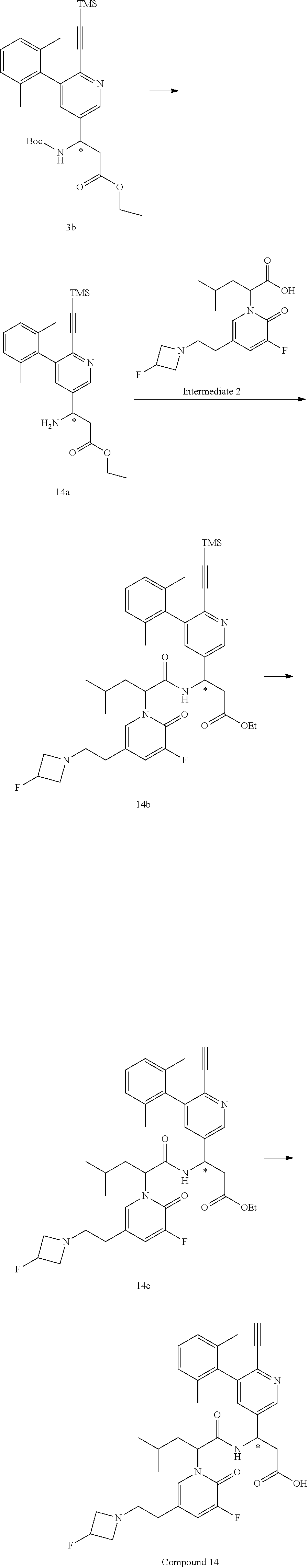 [SMILES: CCOC(=O)CC(NC(=O)OC(C)(C)C)c1cnc(C#C[Si](C)(C)C)c(-c2c(C)cccc2C)c1 ; CCOC(=O)CC(N)c1cnc(C#C[Si](C)(C)C)c(-c2c(C)cccc2C)c1 ; CC(C)CC(C(=O)O)n1cc(CCN2CC(F)C2)cc(F)c1=O ; CCOC(=O)CC(NC(=O)C(CC(C)C)n1cc(CCN2CC(F)C2)cc(F)c1=O)c1cnc(C#C[Si](C)(C)C)c(-c2c(C)cccc2C)c1 ; C#Cc1ncc(C(CC(=O)OCC)NC(=O)C(CC(C)C)n2cc(CCN3CC(F)C3)cc(F)c2=O)cc1-c1c(C)cccc1C ; C#Cc1ncc(C(CC(=O)O)NC(=O)C(CC(C)C)n2cc(CCN3CC(F)C3)cc(F)c2=O)cc1-c1c(C)cccc1C]

Reference can be made to example 3 for the reaction conditions and operations to afford compound 14 (42 mg).

Ms m/z (ESI): 605.3 [M+H]⁺.

¹H NMR (400 MHz, CD₃OD) δ 8.55-8.46 (m, 1H), 7.62-7.50 (m, 1H), 7.45-7.30 (m, 2H), 7.23-7.15 (m, 1H), 7.15-7.07 (m, 2H), 5.71-5.60 (m, 1H), 5.44-5.04 (m, 2H), 4.34-4.12 (m, 1H), 4.04-3.80 (m, 2H), 3.70-3.53 (m, 1H), 3.45 (d, 1H), 3.29-3.20 (m, 1H), 3.07-2.98 (m, 1H), 2.80-2.56 (m, 4H), 2.03-1.92 (m, 6H), 1.90-1.77 (m, 2H), 1.48-1.34 (m, 1H), 0.99-0.87 (m, 6H).

### Example 15: Preparation of Compound 15 and Stereoisomer Thereof

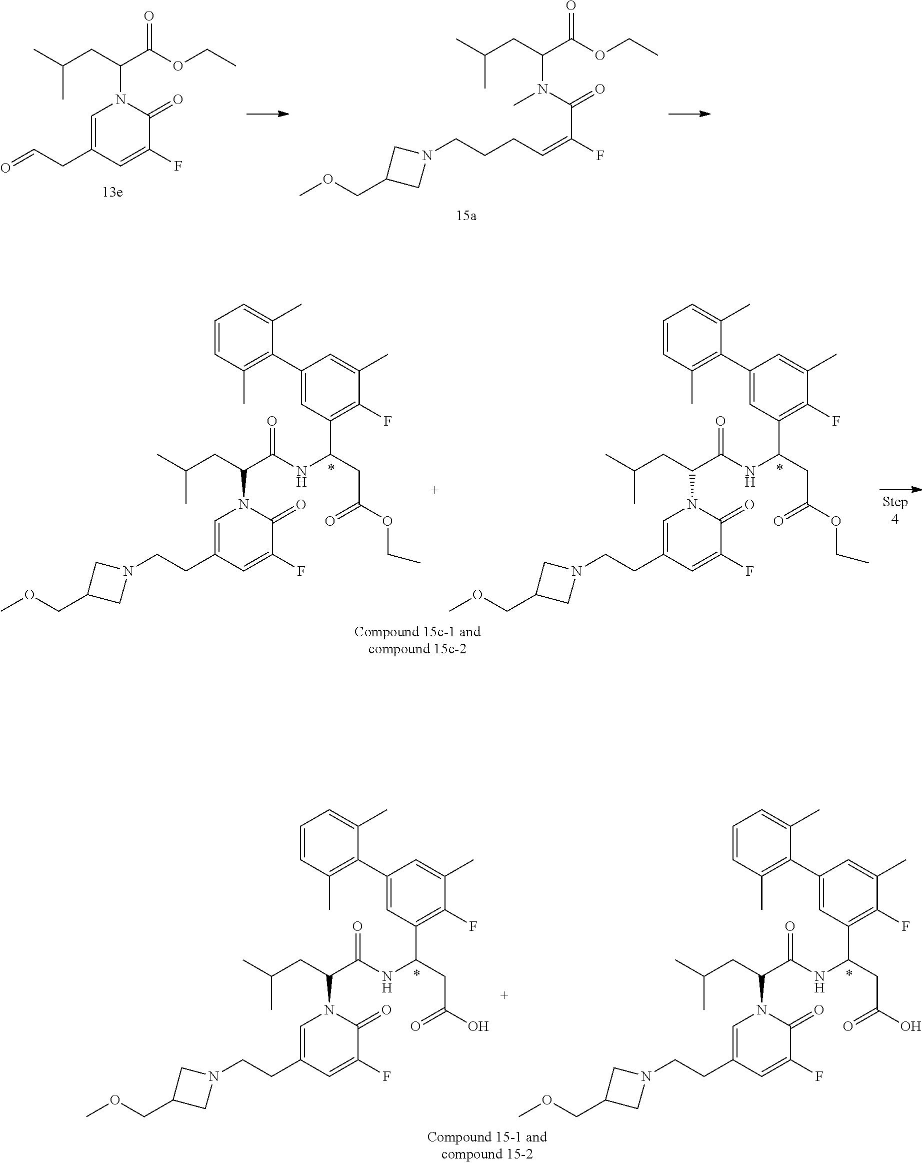

### Step 1: Synthesis of 15a

13e (0.5 g, 1.68 mmol) and 3-methoxymethylazetidine hydrochloride (0.35 g, 2.51 mmol) were dissolved in DCE (10 mL) and the mixture was reacted at room temperature for 20 min. Sodium triacetoxyborohydride (0.71 g, 3.36 mmol) was added in portions and the resulting mixture was reacted at room temperature for 16 h. The reaction mixture was concentrated under reduced pressure to remove the reaction solvent and the residue was subjected to column chromatography on a silica gel chromatographic column (dichloromethane:methanol=50:1-10:1) to afford 15a (0.47 g, yield: 73.47%).

Ms m/z (ESI): 383.2 [M+H]⁺.

### Step 2: Synthesis of 15b

15a (0.47 g, 1.23 mmol) was dissolved in anhydrous ethanol (6 mL) and water (1 mL), lithium hydroxide monohydrate (103 mg, 2.46 mmol) was added, and the resulting mixture was reacted at room temperature for 4 h. The reaction mixture was concentrated under reduced pressure, adjusted with 1 N hydrochloric acid to pH 7, and purified by column chromatography on a C18 reverse phase column (acetonitrile/water-trifluoroacetic acid) to afford 15b (0.38 g, yield: 87.35%).

Ms m/z (ESI): 355.2 [M+H]⁺.

### Step 3: Synthesis of 15c-1 and 15c-2

13m (0.17 g, 0.51 mmol) and 15b (183 mg, 51 mmol) were dissolved in DMF (8 mL), HATU (294 mg, 0.77 mmol) and DIPEA (0.13 g, 1.03 mmol) were added, and the resulting mixture was reacted at room temperature for 16 h. Water (40 mL) was added and the mixture was extracted with ethyl acetate (40 mL×2). The ethyl acetate layers were combined, dried over anhydrous sodium sulfate, filtered and concentrated under reduced pressure and the residue was subjected to column chromatography on a silica gel chromatographic column (dichloromethane:methanol=50:1-10:1) to afford 15c-1 (60 mg, yield: 17.49%, TLC Rf=0.43) and 15c-2 (61 mg, yield: 17.78%, TLC Rf=0.28).

Ms m/z (ESI): 666.3 [M+H]⁺.

### Step 4: Synthesis of Compound 15-1 and Compound 15-2

15c-1 (25 mg, 0.04 mmol) was dissolved in anhydrous ethanol (4 mL) and water (1 mL), lithium hydroxide monohydrate (7 mg, 0.15 mmol) was added, and the resulting mixture was reacted at room temperature for 16 h. The reaction mixture was concentrated under reduced pressure, adjusted with 1 N hydrochloric acid to pH 7, and separated and purified by a C18 reverse phase column to afford compound 15-1 (8 mg, yield: 33.33%).

Ms m/z (ESI): 638.3 [M+H]⁺.

¹H NMR (400 MHz, CD₃OD) δ 7.51 (d, 1H), 7.39-7.28 (m, 1H), 7.15-7.00 (m, 3H), 6.98-6.85 (m, 2H), 5.81-5.74 (m, 1H), 5.66-5.57 (m, 1H), 4.33-3.73 (m, 4H), 3.53-3.34 (m, 7H), 3.15-2.95 (m, 1H), 2.89-2.82 (m, 2H), 2.79-2.62 (m, 2H), 2.35-2.26 (m, 3H), 2.05-1.87 (m, 6H), 1.88-1.71 (m, 2H), 1.45-1.23 (m, 1H), 0.91 (m, 6H).

15c-2 (25 mg, 0.04 mmol) was dissolved in anhydrous ethanol (4 mL) and water (1 mL), lithium hydroxide monohydrate (7 mg, 0.15 mmol) was added, and the resulting mixture was reacted at room temperature for 16 h. The reaction mixture was concentrated under reduced pressure, adjusted with 1 N hydrochloric acid to pH 7, and separated and purified by a C18 reverse phase column to afford compound 15-2 (7 mg, yield: 29.16%).

Ms m/z (ESI): 638.4 [M+H]⁺.

¹H NMR (400 MHz, CD₃OD) δ 7.49 (s, 1H), 7.34-7.26 (m, 1H), 7.14-7.00 (m, 3H), 6.93-6.81 (m, 2H), 5.86-5.40 (m, 2H), 4.28-3.92 (m, 4H), 3.48-3.45 (m, 2H), 3.43 (s, 3H), 3.39-3.34 (m, 2H), 3.03 (s, 1H), 2.86-2.79 (m, 2H), 2.75-2.66 (m, 2H), 2.28 (s, 3H), 2.00-1.90 (m, 5H), 1.84 (s, 3H), 1.50-1.35 (m, 1H), 0.99-0.89 (m, 6H).

### Example 16: Preparation of Compound 16 and Stereoisomer Thereof

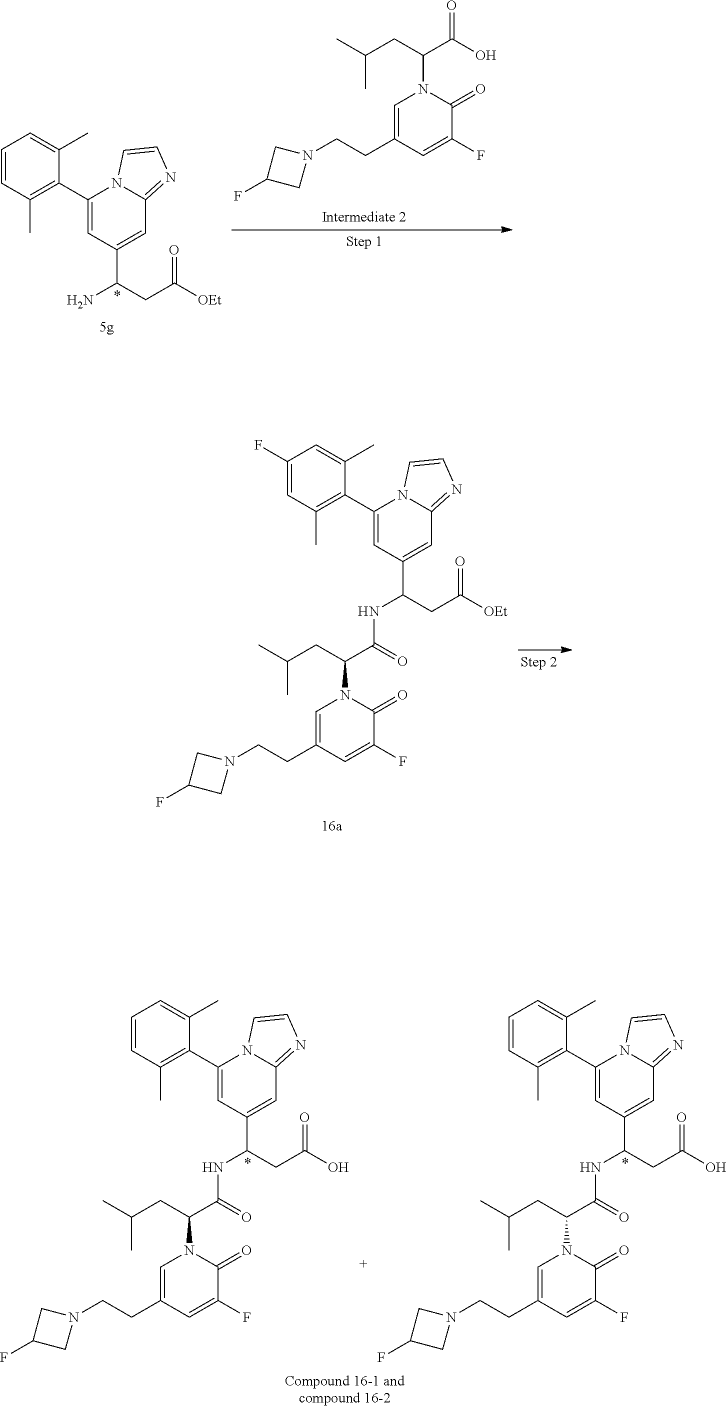 [SMILES: CCOC(=O)CC(N)c1cc(-c2c(C)cccc2C)n2ccnc2c1 ; CC(C)CC(C(=O)O)n1cc(CCN2CC(F)C2)cc(F)c1=O ; CCOC(=O)CC(NC(=O)[C@H](CC(C)C)n1cc(CCN2CC(F)C2)cc(F)c1=O)c1cc(-c2c(C)cc(F)cc2C)n2ccnc2c1 ; Cc1cccc(C)c1-c1cc(C(CC(=O)O)NC(=O)[C@H](CC(C)C)n2cc(CCN3CC(F)C3)cc(F)c2=O)cc2nccn12 ; Cc1cccc(C)c1-c1cc(C(CC(=O)O)NC(=O)[C@@H](CC(C)C)n2cc(CCN3CC(F)C3)cc(F)c2=O)cc2nccn12]

### Step 1: Synthesis of 16a

Under nitrogen protection, 5g (71 mg, 0.21 mmol) and intermediate 2 (76 mg, 0.23 mmol) were added to a reaction flask, dry DMF (3.0 mL) was added and dissolved, HATU (160 mg, 0.42 mmol) and DIPEA (160 mg, 1.26 mmol) were added. The mixture was then stirred at room temperature overnight, ethyl acetate (40 mL) was added, and the resulting mixture was washed with water twice (10 mL×2), washed with saturated brine once (10 mL), dried over anhydrous sodium sulfate and filtered. The filtrate was concentrated under reduced pressure and then subjected to silica gel column chromatography (methanol/dichloromethane: 0%-8%) to afford 16a (95 mg, yield: 69.84%).

Ms m/z (ESI): 648.3 [M+H]⁺.

### Step 2: Synthesis of Compound 16-1 and Compound 16-2

16a (75 mg, 0.12 mmol) was dissolved in THF (2.0 mL) and water (0.7 mL), lithium hydroxide monohydrate (15 mg, 0.36 mmol) was added, and the resulting mixture was reacted at room temperature for 6 h. The reaction mixture was adjusted with 1N hydrochloric acid to pH 5-6, concentrated under reduced pressure, and subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compounds 16-1 (18 mg, retention time=1.069 min, 24.21%) and 16-2 (18 mg, retention time=1.423 min, 24.21%).

Compound 16-1: Ms m/z (ESI): 620.2 [M+H]⁺.

¹H NMR (400 MHz, CDCl₃) δ 8.43 (d, 1H), 8.25 (s, 1H), 7.67 (s, 1H), 7.43 (s, 1H), 7.23-7.17 (m, 1H), 7.15-7.07 (m, 2H), 7.03-6.96 (m, 2H), 6.84 (d, 1H), 5.64 (s, 1H), 5.25-5.01 (m, 2H), 4.26-4.14 (m, 1H), 3.84-3.72 (m, 1H), 3.45-3.42 (m, 1H), 3.23-3.08 (m, 1H), 3.07-2.98 (m, 2H), 2.78-2.55 (m, 3H), 2.47-2.35 (m, 1H), 2.11-2.03 (m, 1H), 2.02 (s, 3H), 1.97 (s, 3H), 1.87-1.77 (m, 1H), 1.48-1.36 (m, 1H), 0.93-0.88 (m, 6H).

Compound 16-2: Ms m/z (ESI): 620.2 [M+H]⁺.

¹H NMR (400 MHz, CDCl₃) δ 8.15-8.11 (m, 1H), 7.65-7.57 (m, 2H), 7.24-7.18 (m, 1H), 7.15-7.10 (m, 2H), 7.04-6.84 (m, 4H), 5.78-5.72 (m, 1H), 5.61-5.53 (m, 1H), 5.37-5.16 (m, 1H), 4.19-4.08 (m, 1H), 4.07-3.96 (m, 1H), 3.47-3.28 (m, 2H), 3.11-2.96 (m, 2H), 2.67-2.57 (m, 2H), 2.56-2.42 (m, 2H), 2.09-2.04 (m, 1H), 2.04-2.01 (m, 6H), 1.84-1.75 (m, 1H), 1.48-1.38 (m, 1H), 0.95-0.88 (m, 6H).

### Example 17: Preparation of Compound 17 and Stereoisomer Thereof

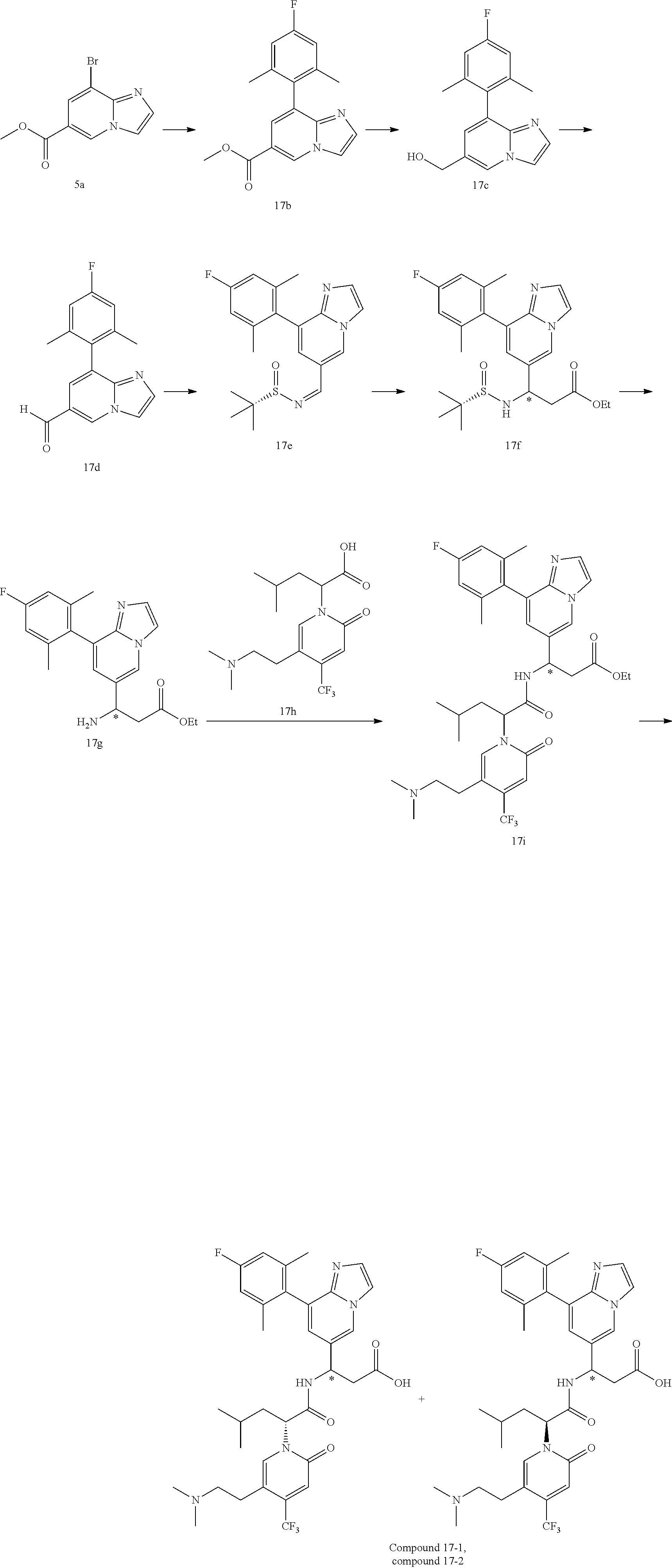

For the ammonium salt of compound 17-1 (42 mg, retention time=1.360 min) and ammonium salt of compound 17-2 (65 mg, retention time=2.093 min), reference can be made to example 5 for the reaction conditions and operations.

SFC resolution conditions: instrument: Waters 150 SFC; chromatographic column: Chiralpak Column (250*30 mm*10 um; mobile phases: A for CO₂; B for methanol solution containing 0.1% ammonia water); elution condition: isocratic elution using 45% B; flow rate: 100 mL/min.

Ammonium salt of compound 17-1: LC-Ms m/z (ESI): 658.2[M+H]⁺.

¹H NMR (400 MHz, DMSO-d₆) δ 9.04 (d, 1H), 8.48 (s, 1H), 7.91 (s, 1H), 7.82 (s, 1H), 7.45 (s, 1H), 7.04-6.91 (m, 3H), 6.68 (s, 1H), 5.56-5.46 (m, 1H), 5.26-5.15 (m, 1H), 2.82 (d, 2H), 2.60-2.52 (m, 2H), 2.39-2.28 (m, 2H), 2.15 (s, 6H), 1.99-1.91 (m, 1H), 1.89 (s, 3H), 1.85 (s, 3H), 1.83-1.71 (m, 1H), 1.40-1.28 (m, 1H), 0.89 (d, 3H), 0.84 (d, 3H).

Ammonium salt of compound 17-2: LC-Ms m/z (ESI): 658.2[M+H]⁺.

¹H NMR (400 MHz, DMSO-d₆) δ 9.09 (d, 1H), 8.53 (s, 1H), 8.01 (s, 1H), 7.85 (s, 1H), 7.49 (s, 1H), 7.03 (d, 3H), 6.76 (s, 1H), 5.64-5.53 (m, 1H), 5.22-5.11 (m, 1H), 2.86-2.73 (m, 2H), 2.70-2.61 (m, 2H), 2.61-2.52 (m, 2H), 2.31 (s, 6H), 1.96 (s, 3H), 1.94 (s, 3H), 1.87-1.77 (m, 1H), 1.65-1.53 (m, 1H), 1.21-1.12 (m 1H), 0.82-0.69 (m, 6H).

### Example 18: Preparation of Compound 18 and Stereoisomer Thereof

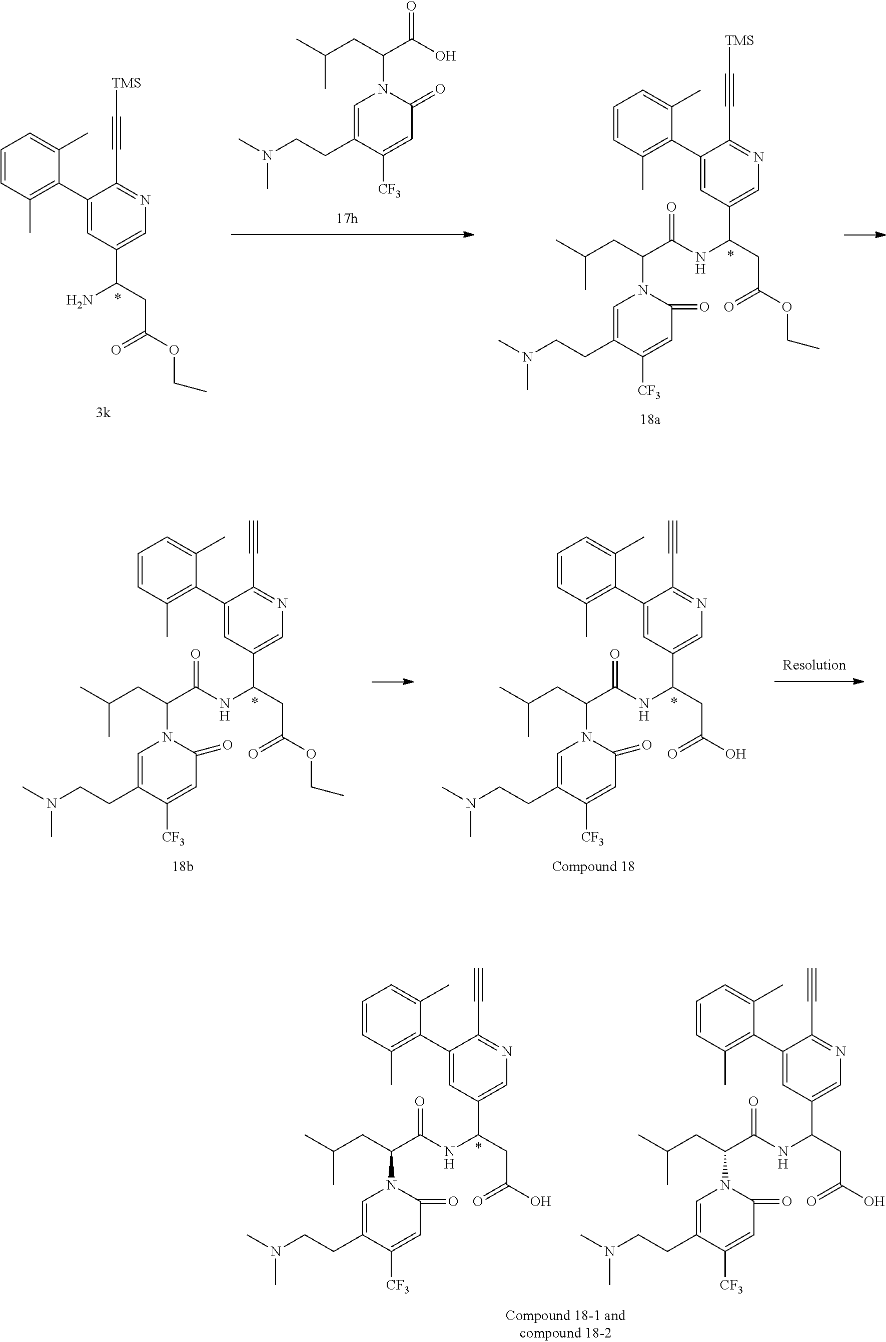

For the trifluoroacetate of compound 18-1 (17 mg, retention time=5.081 min) and trifluoroacetate of compound 18-2 (15 mg, retention time=5.132 min), reference can be made to example 3 for the reaction conditions and operations.

HPLC preparation conditions: instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid).

Trifluoroacetate of compound 18-1: LC-Ms m/z (ESI): 625.3 [M+H]⁺.

¹H NMR (400 MHz, CDCl₃) δ 8.70 (s, 1H), 7.69 (s, 1H), 7.59 (s, 1H), 7.52-7.40 (m, 1H), 7.26-7.18 (m, 1H), 7.15-7.07 (m, 2H), 6.90 (s, 1H), 5.73-5.65 (m, 1H), 5.45-5.35 (m, 1H), 3.52-3.40 (m, 1H), 3.19-2.94 (m, 5H), 2.92-2.79 (m, 7H), 2.03-1.81 (m, 8H), 1.46-1.33 (m, 1H), 0.96-0.82 (m, 6H).

Trifluoroacetate of compound 18-2: LC-Ms m/z (ESI): 625.3 [M+H]⁺.

¹H NMR (400 MHz, CDCl₃) δ 8.62 (s, 1H), 7.96-7.77 (m, 1H), 7.53-7.42 (m, 2H), 7.24-7.17 (m, 1H), 7.14-7.07 (m, 2H), 6.85 (m, 1H), 5.74-5.64 (m, 1H), 5.61-5.51 (m, 1H), 3.64-3.51 (m, 1H), 3.30-3.19 (m, 1H), 3.08-2.97 (m, 1H), 2.95-2.82 (m, 9H), 2.76-2.66 (m, 1H), 2.09-2.01 (m, 1H), 1.98 (m, 6H), 1.63-1.52 (m, 1H), 1.42-1.30 (m, 1H), 0.89-0.75 (m, 6H).

### Example 19: Preparation of Compound 19 and Stereoisomer Thereof

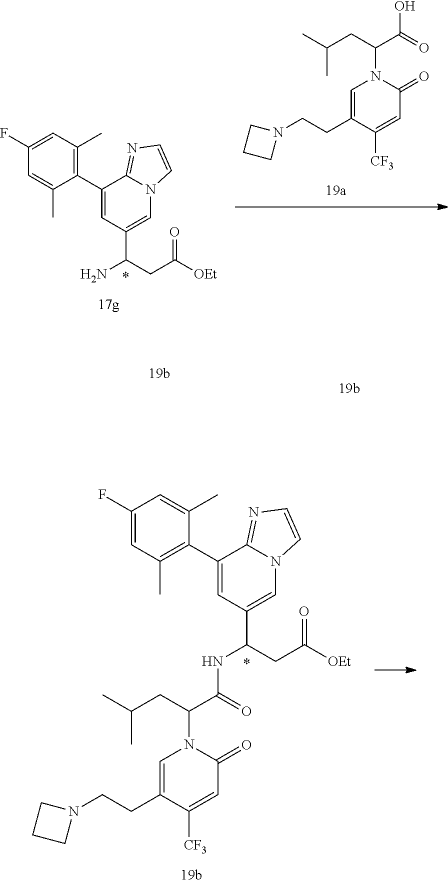 [SMILES: CCOC(=O)CC(N)c1cc(-c2c(C)cc(F)cc2C)c2nccn2c1 ; CC(C)CC(C(=O)O)n1cc(CCN2CCC2)c(C(F)(F)F)cc1=O ; CCOC(=O)CC(NC(=O)C(CC(C)C)n1cc(CCN2CCC2)c(C(F)(F)F)cc1=O)c1cc(-c2c(C)cc(F)cc2C)c2nccn2c1]

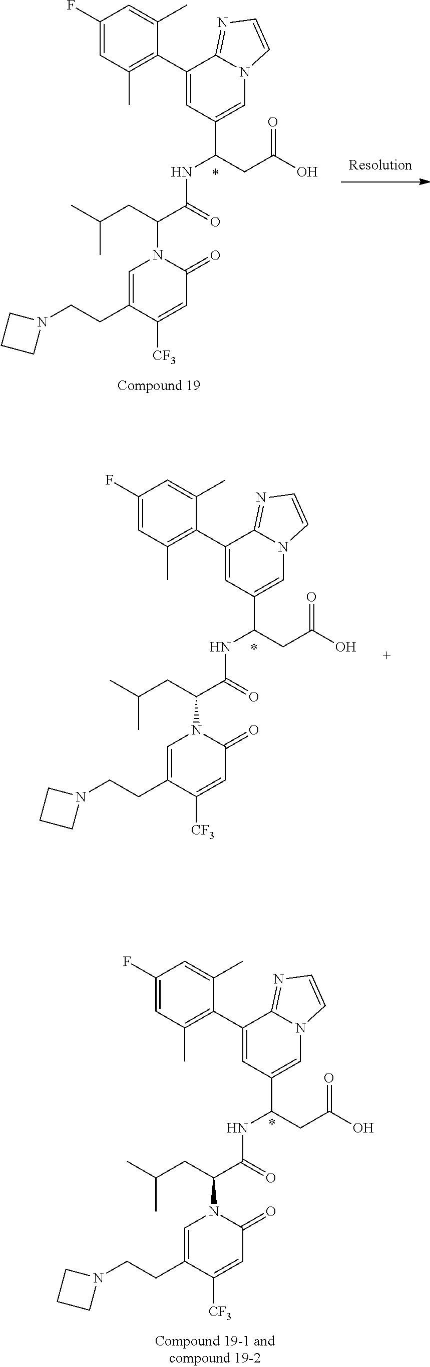 [SMILES: Cc1cc(F)cc(C)c1-c1cc(C(CC(=O)O)NC(=O)C(CC(C)C)n2cc(CCN3CCC3)c(C(F)(F)F)cc2=O)cn2ccnc12 ; Cc1cc(F)cc(C)c1-c1cc(C(CC(=O)O)NC(=O)[C@@H](CC(C)C)n2cc(CCN3CCC3)c(C(F)(F)F)cc2=O)cn2ccnc12 ; Cc1cc(F)cc(C)c1-c1cc(C(CC(=O)O)NC(=O)[C@H](CC(C)C)n2cc(CCN3CCC3)c(C(F)(F)F)cc2=O)cn2ccnc12]

### Step 1: Synthesis of 19b

Under nitrogen protection, 17g (138 mg, 0.39 mmol) and 19a (see WO 2021076890A1 for the synthetic method) (140 mg, 0.39 mmol) were added to a reaction flask, followed by dry DMF (2.5 mL), HATU (297 mg, 0.78 mmol) and DIPEA (302 mg, 2.34 mmol), and the resulting mixture was stirred at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to silica gel column chromatography to afford 19b (128 mg, yield: 47%).

LC-Ms m/z (ESI): 698.3 [M+H]⁺.

### Step 2: Synthesis of Compound 19-1 and Compound 19-2

19b (126 mg, 0.18 mmol) was dissolved in THF (2.5 mL) and water (0.5 mL), lithium hydroxide monohydrate (23 mg, 0.55 mmol) was added, and the resulting mixture was reacted at room temperature for 6 h. The reaction mixture was adjusted with 1 N hydrochloric acid to pH 5-6 and concentrated under reduced pressure to remove the solvent to afford a crude of compound 19, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 19-1 (25 mg, retention time=3.034 min, yield: 20.74%) and compound 19-2 (20 mg, retention time=3.112 min, yield: 16.60%).

Compound 19-1: LC-Ms m/z (ESI): 670.3 [M+H]⁺.

Compound 19-2: LC-Ms m/z (ESI): 670.3 [M+H]⁺.

¹H NMR (400 MHz, DMSO-d₆) δ 9.06 (d, 1H), 8.52 (d, 1H), 7.99 (d, 1H), 7.75 (s, 1H), 7.48 (d, 1H), 7.08-6.97 (m, 3H), 6.75 (s, 1H), 5.56-5.49 (m, 1H), 5.23-5.14 (m, 1H), 3.28-3.17 (m, 4H), 2.78-2.72 (m, 2H), 2.63-2.54 (m, 2H), 2.47-2.40 (m, 1H), 2.02-1.94 (m 1H), 1.95 (s, 3H), 1.94 (s, 3H), 1.27-1.19 (m, 1H), 1.85-1.78 (m, 1H), 1.69-1.60 (m, 1H), 0.82-0.71 (m, 6H).

### Example 20: Preparation of Compound 20 and Stereoisomer Thereof

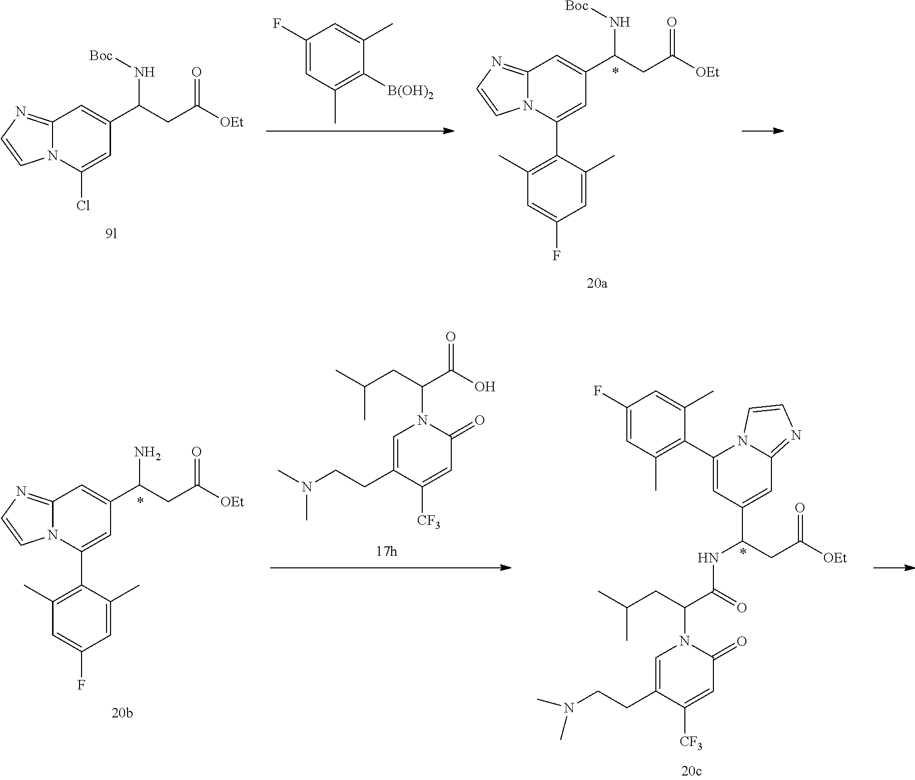 [SMILES: CCOC(=O)CC(NC(=O)OC(C)(C)C)c1cc(Cl)n2ccnc2c1 ; Cc1cc(F)cc(C)c1B(O)O ; CCOC(=O)CC(NC(=O)OC(C)(C)C)c1cc(-c2c(C)cc(F)cc2C)n2ccnc2c1 ; CCOC(=O)CC(N)c1cc(-c2c(C)cc(F)cc2C)n2ccnc2c1 ; CC(C)CC(C(=O)O)n1cc(CCN(C)C)c(C(F)(F)F)cc1=O ; CCOC(=O)CC(NC(=O)C(CC(C)C)n1cc(CCN(C)C)c(C(F)(F)F)cc1=O)c1cc(-c2c(C)cc(F)cc2C)n2ccnc2c1]

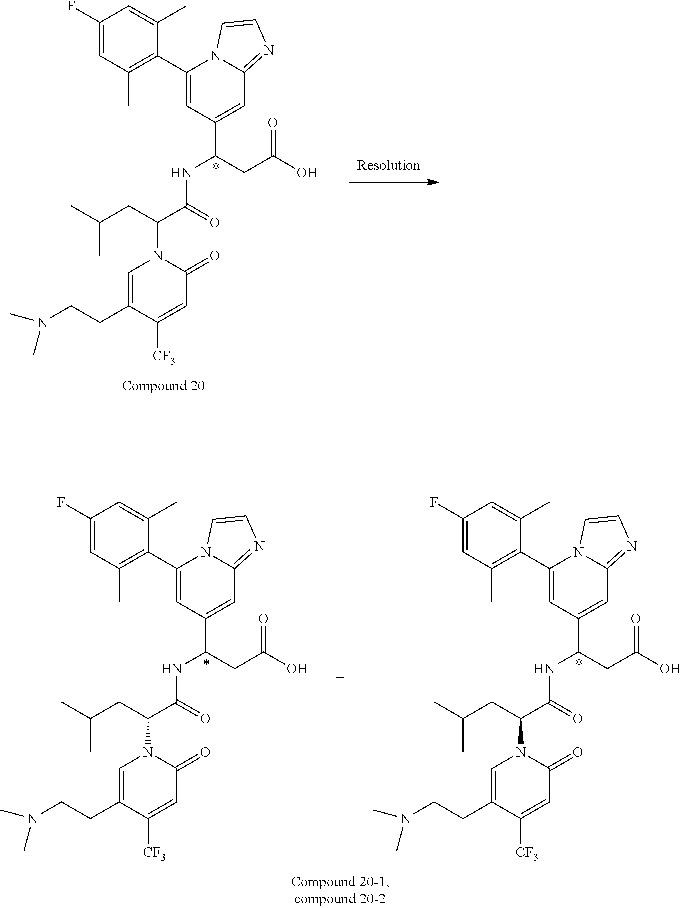 [SMILES: Cc1cc(F)cc(C)c1-c1cc(C(CC(=O)O)NC(=O)C(CC(C)C)n2cc(CCN(C)C)c(C(F)(F)F)cc2=O)cc2nccn12 ; Cc1cc(F)cc(C)c1-c1cc(C(CC(=O)O)NC(=O)[C@@H](CC(C)C)n2cc(CCN(C)C)c(C(F)(F)F)cc2=O)cc2nccn12 ; Cc1cc(F)cc(C)c1-c1cc(C(CC(=O)O)NC(=O)[C@H](CC(C)C)n2cc(CCN(C)C)c(C(F)(F)F)cc2=O)cc2nccn12]

For the trifluoroacetate of compound 20-1 (42 mg, retention time=3.084 min, yield: 15.20%) and trifluoroacetate of compound 20-2 (52 mg, retention time=3.171 min, yield: 18.82%), reference can be made to example 9 for the reaction conditions and operations.

HPLC preparation conditions: instrument: waters 2767 preparative chromatographic column: SunFire@Prep C18 (19 mm×150 mm), composition of mobile phases: mobile phase A: acetonitrile, and mobile phase B: water (containing 0.1% trifluoroacetic acid).

Trifluoroacetate of compound 20-1: LC-Ms m/z (ESI): 658.2 [M+H]⁺.

¹H NMR (400 MHz, DMSO-d₆) δ 9.76 (s, 1H), 9.26 (d, 1H), 8.07 (s, 1H), 7.94 (s, 1H), 7.79 (s, 1H), 7.45 (d, 1H), 7.31 (s, 1H), 7.22-7.15 (m, 2H), 6.73 (s, 1H), 5.64-5.57 (m, 1H), 5.38-5.29 (m, 1H), 3.18-3.07 (m, 3H), 2.97-2.86 (m, 2H), 2.83 (s, 7H), 2.05-1.95 (m, 1H), 1.94 (s, 3H), 1.86 (s, 4H), 1.44-1.34 (m, 1H), 0.92 (d, 3H), 0.88 (d, 3H).

Trifluoroacetate of compound 20-2: LC-Ms m/z (ESI): 658.3 [M+H]⁺.

¹H NMR (400 MHz, DMSO-d₆) δ 9.76 (s, 1H), 9.29 (d, 1H), 8.04 (s, 1H), 7.92 (s, 1H), 7.81 (s, 1H), 7.45 (s, 1H), 7.33-7.17 (m, 3H), 6.83 (s, 1H), 5.71-5.64 (m, 1H), 5.35-5.27 (m, 1H), 3.19-3.02 (m, 2H), 2.93-2.76 (m, 10H), 1.96 (d, 6H), 1.91-1.79 (m, 1H), 1.76-1.67 (m, 1H), 1.28-1.16 (m, 1H), 0.87-0.76 (m, 6H).

### Example 21: Preparation of Compound 21 and Stereoisomer Thereof

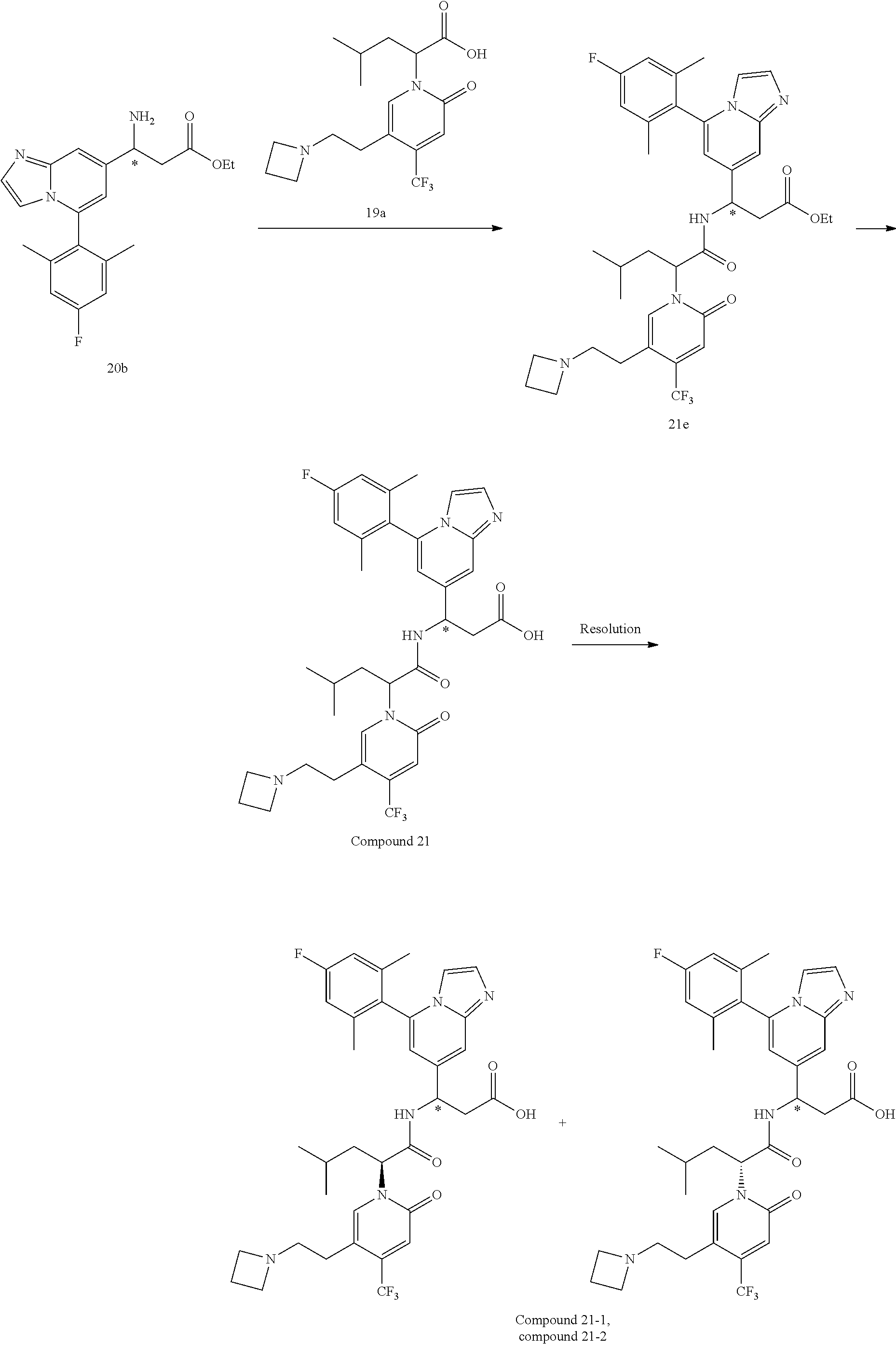 [SMILES: CCOC(=O)CC(N)c1cc(-c2c(C)cc(F)cc2C)n2ccnc2c1 ; CC(C)CC(C(=O)O)n1cc(CCN2CCC2)c(C(F)(F)F)cc1=O ; CCOC(=O)CC(NC(=O)C(CC(C)C)n1cc(CCN2CCC2)c(C(F)(F)F)cc1=O)c1cc(-c2c(C)cc(F)cc2C)n2ccnc2c1 ; Cc1cc(F)cc(C)c1-c1cc(C(CC(=O)O)NC(=O)C(CC(C)C)n2cc(CCN3CCC3)c(C(F)(F)F)cc2=O)cc2nccn12 ; Cc1cc(F)cc(C)c1-c1cc(C(CC(=O)O)NC(=O)[C@H](CC(C)C)n2cc(CCN3CCC3)c(C(F)(F)F)cc2=O)cc2nccn12 ; Cc1cc(F)cc(C)c1-c1cc(C(CC(=O)O)NC(=O)[C@@H](CC(C)C)n2cc(CCN3CCC3)c(C(F)(F)F)cc2=O)cc2nccn12]

### Step 1: Synthesis of 21a

Under nitrogen protection, crude 20b (184 mg, 0.51 mmol) and 19a (181 mg, 0.51 mmol) were added to a reaction flask, followed by dry DMF (4.0 mL), HATU (389 mg, 1.02 mmol) and DIPEA (395 mg, 3.06 mmol). The reaction mixture was stirred at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 21a (234 mg, yield: 65.76%).

LC-Ms m/z (ESI): 349.8 [M+2H]²⁺.

### Step 2: Synthesis of Compounds 21-1 and 21-2

21a (209 mg, 0.30 mmol) was dissolved in THF (3 mL) and water (1.0 mL), lithium hydroxide monohydrate (38 mg, 0.9 mmol) was added, and the resulting mixture was reacted at room temperature for 3 h. The reaction mixture was adjusted with 1 N hydrochloric acid to pH 5-6 and concentrated under reduced pressure to afford a crude of compound 20, which was directly subjected to preparative HPLC (instrument: waters 2767 preparative chromatographic column: SunFire@Prep C18 (19 mmx 150 mm); composition of mobile phases: mobile phase A: acetonitrile, and mobile phase B: water (containing 0.1% ammonium acetate)) to afford compound 21-1 (20 mg, retention time=3.177 min, yield: 9.95%) and compound 21-2 (26 mg, retention time=3.249 min, yield: 12.94%).

Compound 21-1: LC-Ms m/z (ESI): 670.3 [M+H]⁺.

¹H NMR (400 MHz, DMSO-d₆) δ 9.23 (d, 1H), 7.82 (s, 1H), 7.52 (s, 1H), 7.44 (s, 1H), 7.12 (d, 2H), 7.00 (s, 1H), 6.74 (d, 1H), 6.70 (s, 1H), 5.57-5.48 (m, 1H), 5.24-5.16 (m, 1H), 3.22-3.18 (m, 4H), 2.82-2.67 (m, 3H), 2.57-2.49 (m, 2H), 2.46-2.38 (m, 1H), 2.04-1.93 (m, 2H), 1.91 (s, 3H), 1.87 (s, 3H), 1.86-1.80 (m, 2H), 1.42-1.34 (m, 1H), 0.91 (d, 3H), 0.86 (d, 3H).

Compound 21-2: LC-Ms m/z (ESI): 670.3 [M+H]⁺.

¹H NMR (400 MHz, DMSO-d₆) δ 9.13 (d, 1H), 7.77 (s, 1H), 7.54 (s, 1H), 7.47 (s, 1H), 7.17 (s, 1H), 7.15 (s, 1H), 7.03 (s, 1H), 6.78 (s, 1H), 6.76 (s, 1H), 5.59-5.52 (m, 1H), 5.27-5.20 (m, 1H), 3.31-3.19 (m, 3H), 2.79-2.52 (m, 6H), 2.00 (q, J=7.2 Hz, 2H), 1.95 (s, 6H), 1.91-1.81 (m, 1H), 1.78-1.66 (m 2H), 1.32-1.20 (m, 1H), 0.86-0.74 (m, 6H).

### Example 22: Preparation of Compound 22 and Stereoisomer Thereof

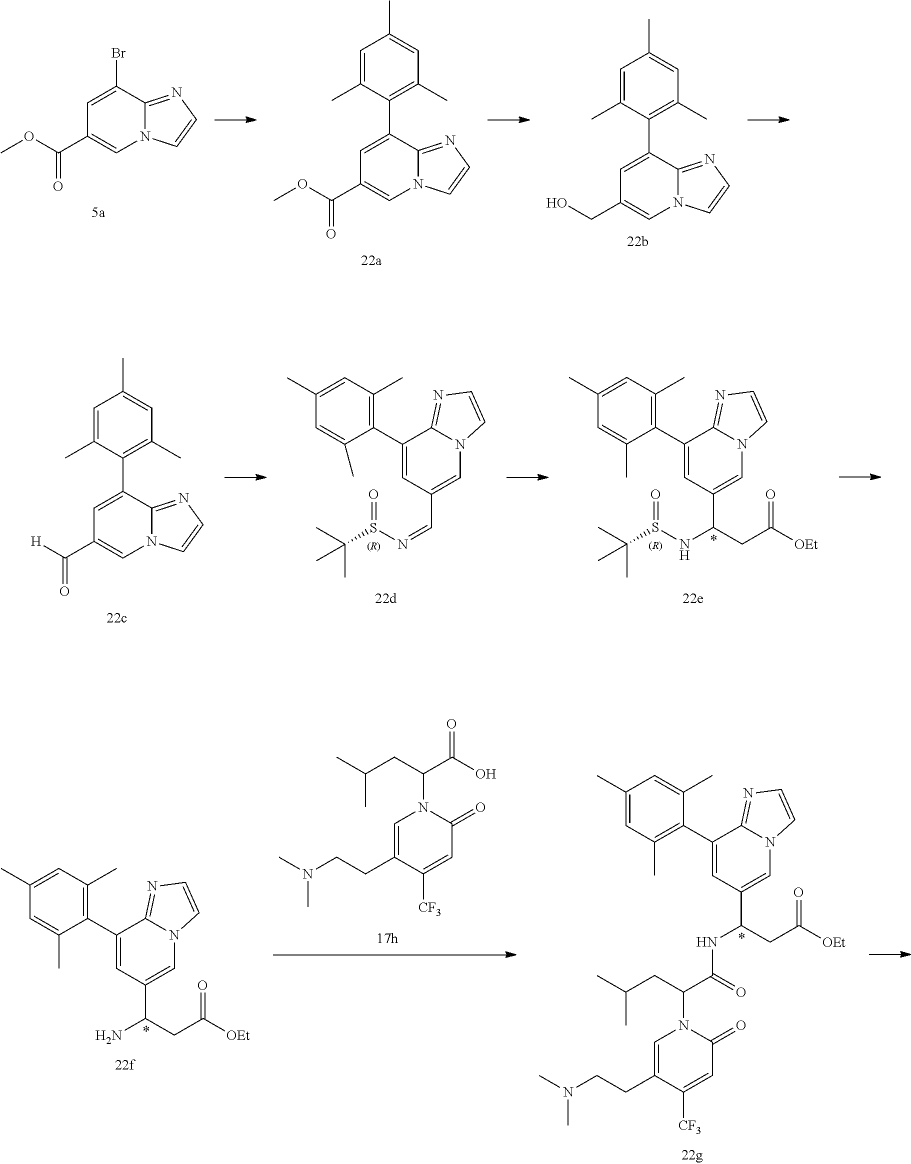

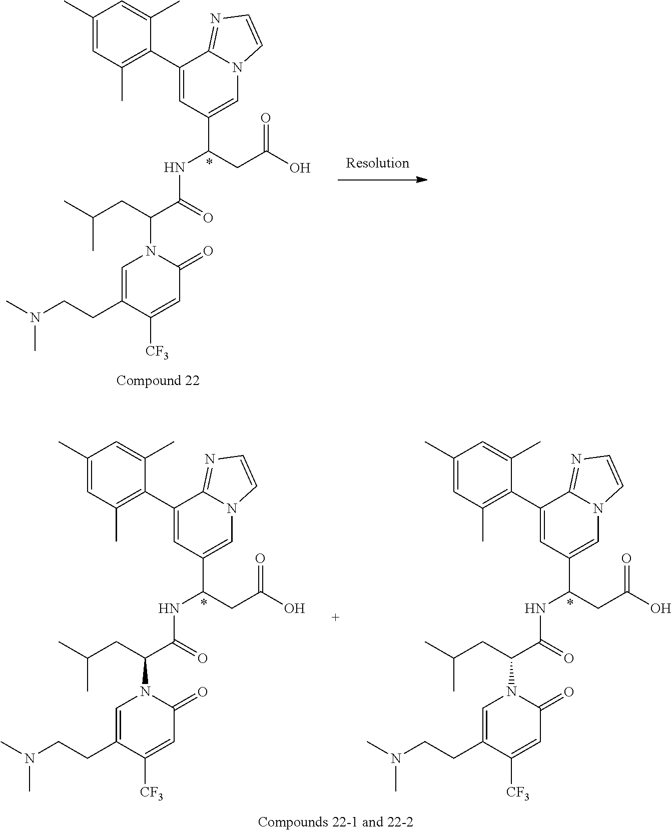 [SMILES: Cc1cc(C)c(-c2cc(C(CC(=O)O)NC(=O)C(CC(C)C)n3cc(CCN(C)C)c(C(F)(F)F)cc3=O)cn3ccnc23)c(C)c1 ; Cc1cc(C)c(-c2cc(C(CC(=O)O)NC(=O)[C@H](CC(C)C)n3cc(CCN(C)C)c(C(F)(F)F)cc3=O)cn3ccnc23)c(C)c1 ; Cc1cc(C)c(-c2cc(C(CC(=O)O)NC(=O)[C@@H](CC(C)C)n3cc(CCN(C)C)c(C(F)(F)F)cc3=O)cn3ccnc23)c(C)c1]

For the trifluoroacetate of compound 22-1 (30 mg, retention time=3.076 min) and trifluoroacetate of compound 22-2 (34 mg, retention time=3.158 min), reference can be made to example 5 for the reaction conditions and operations.

HPLC preparation conditions: instrument: waters 2767 preparative chromatographic column: SunFire@Prep C18 (19 mm×150 mm), composition of mobile phases: mobile phase A: acetonitrile, and mobile phase B: water (containing 0.1% trifluoroacetic acid).

Trifluoroacetate of compound 22-1: LC-Ms m/z (ESI): 654.4 [M+H]⁺.

¹H NMR (400 MHz, DMSO-d₆) δ 9.71 (s, 1H), 9.19 (d, 1H), 8.86 (s, 1H), 8.35 (s, 1H), 8.05 (s, 1H), 7.93 (s, 1H), 7.61 (s, 1H), 7.06 (s), 7.04 (s, 1H), 6.72 (s, 1H), 5.55-5.48 (m, 1H), 5.34-5.25 (m, 1H), 3.12 (s, 2H), 2.92 (d, J=7.3 Hz, 3H), 2.83 (d, J=3.7 Hz, 7H), 2.34 (s, 3H), 2.05-1.93 (m, 2H), 1.88 (s, 3H), 1.86-1.82 (m, 1H), 1.80 (s, 3H), 1.41-1.32 (m, 1H), 0.91 (d, 3H), 0.87 (d, 3H).

Trifluoroacetate of compound 22-2: LC-Ms m/z (ESI): 654.4 [M+H]⁺.

¹H NMR (400 MHz, DMSO-d₆) δ 9.72 (s, 1H), 9.25 (d, 1H), 8.90 (s, 1H), 8.39 (s, 1H), 8.04 (s, 1H), 7.92 (s, 1H), 7.62 (s, 1H), 7.08 (s, 2H), 6.83 (s, 1H), 5.65-5.58 (m, 1H), 5.30-5.22 (m, 1H), 3.18-3.01 (m, 3H), 2.96-2.89 (m, 3H), 2.88-2.79 (m, 8H), 2.34 (s, 3H), 1.91 (s, 6H), 1.87-1.74 (m, 2H), 1.67-1.56 (m, 1H), 1.23-1.12 (m, 1H), 0.81-0.74 (m, 6H).

### Example 23: Preparation of Compound 23 and Stereoisomer Thereof

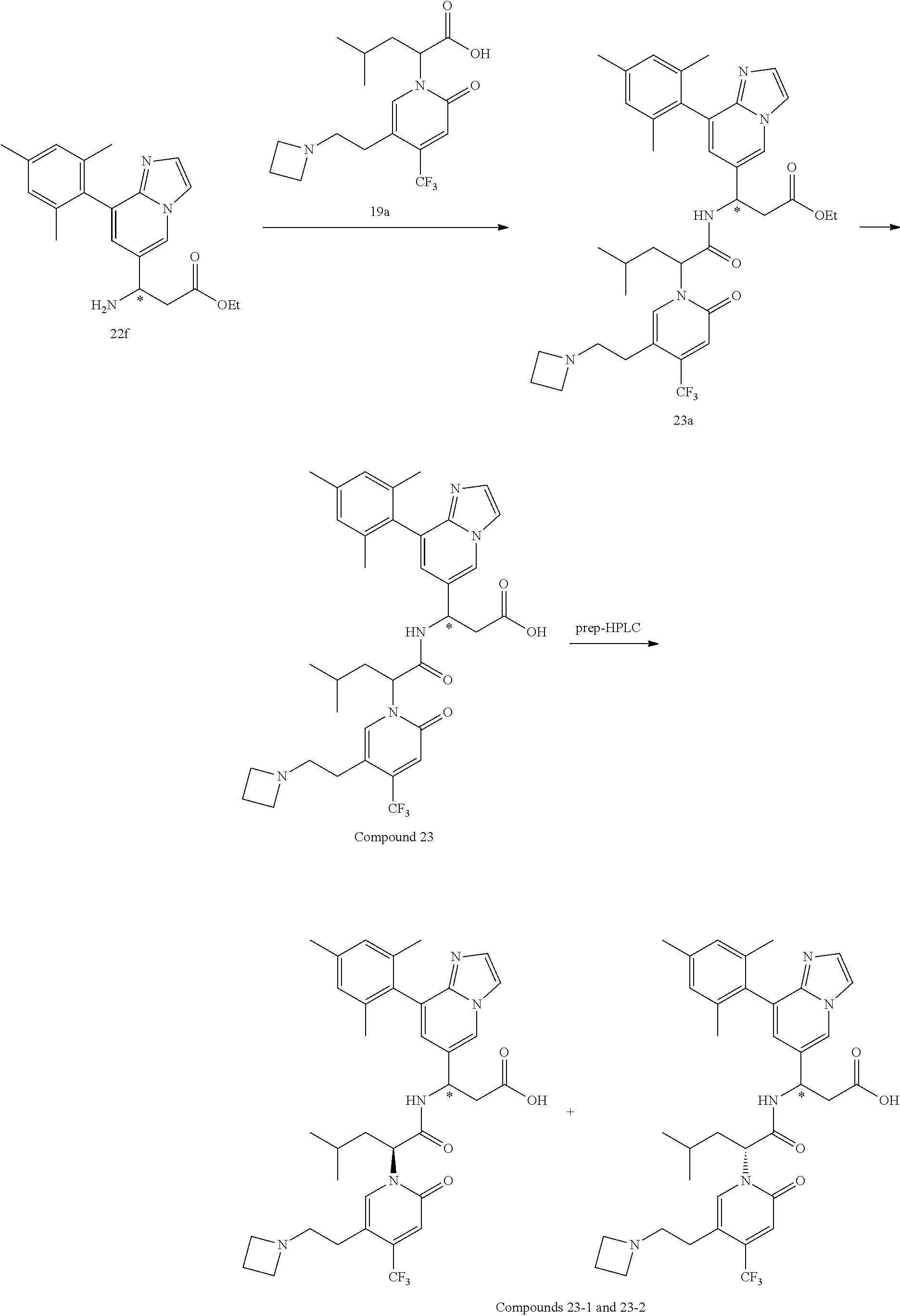 [SMILES: CCOC(=O)CC(N)c1cc(-c2c(C)cc(C)cc2C)c2nccn2c1 ; CC(C)CC(C(=O)O)n1cc(CCN2CCC2)c(C(F)(F)F)cc1=O ; CCOC(=O)CC(NC(=O)C(CC(C)C)n1cc(CCN2CCC2)c(C(F)(F)F)cc1=O)c1cc(-c2c(C)cc(C)cc2C)c2nccn2c1 ; Cc1cc(C)c(-c2cc(C(CC(=O)O)NC(=O)C(CC(C)C)n3cc(CCN4CCC4)c(C(F)(F)F)cc3=O)cn3ccnc23)c(C)c1 ; Cc1cc(C)c(-c2cc(C(CC(=O)O)NC(=O)[C@H](CC(C)C)n3cc(CCN4CCC4)c(C(F)(F)F)cc3=O)cn3ccnc23)c(C)c1 ; Cc1cc(C)c(-c2cc(C(CC(=O)O)NC(=O)[C@@H](CC(C)C)n3cc(CCN4CCC4)c(C(F)(F)F)cc3=O)cn3ccnc23)c(C)c1]

### Step 1: Synthesis of 23a

Under nitrogen protection, 19a (148 mg, 0.41 mmol) was dissolved in dry DMF (4 mL), HATU (312 mg, 0.82 mmol) and DIPEA (318 mg, 2.46 mmol) were added, and the resulting mixture was stirred at room temperature for 40 min. Crude 22f (144 mg, 0.41 mmol) was added and the mixture was reacted at room temperature overnight. Ethyl acetate (80 mL) was added and the resulting reaction mixture was successively washed with water (20 mL×2) and saturated brine (20 mL×1), dried over anhydrous sodium sulfate and filtered. The filtrate was concentrated under reduced pressure and subjected to column chromatography on a silica gel column to afford 23a (132 mg, yield: 46.4%).

LC-Ms m/z (ESI): 347.8 [M+2H]²⁺/2.

### Step 2: Synthesis of Compounds 23-1 and 23-2

23a (180 mg, 0.26 mmol) was dissolved in THF (3 mL) and water (0.8 mL), lithium hydroxide monohydrate (33 mg, 0.78 mmol) was added, and the resulting mixture was reacted at room temperature for 5 h. The reaction mixture was adjusted with 1 N hydrochloric acid to pH 5-6 and concentrated under reduced pressure to afford a crude of compound 23, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 23-1 (29 mg, retention time=3.112 min, yield: 16.75%) and compound 23-2 (31 mg, retention time=3.201 min, yield: 17.91%).

Compound 23-1: LC-Ms m/z (ESI): 666.4 [M+H]⁺.

¹H NMR (400 MHz, DMSO-d₆) δ 9.15 (d, 1H), 8.44 (s, 1H), 7.91 (s, 1H), 7.79 (s, 1H), 7.44 (s, 1H), 6.94-6.91 (m, 3H), 6.70 (s, 1H), 5.51-5.42 (m, 1H), 5.23-5.12 (m, 1H), 3.20-3.18 (m, 5H), 2.77 (d, 2H), 2.51-2.33 (m, 3H), 2.29 (s, 3H), 1.98-1.89 (m, 5H), 1.84 (s, 3H), 1.82-1.77 (m, 1H), 1.42-1.32 (m, 2H), 0.90 (d, 3H), 0.85 (d, 3H).

Compound 23-2: LC-Ms m/z (ESI): 666.4 [M+H]⁺.

¹H NMR (400 MHz, DMSO-d₆) δ 9.12 (d, 1H), 8.48 (d, 1H), 7.97 (s, 1H), 7.75 (s, 1H), 7.46 (s, 1H), 6.96 (d, 3H), 6.75 (s, 1H), 5.57-5.50 (m, 1H), 5.21-5.10 (m, 1H), 3.23-3.17 (m, 3H), 2.74-2.66 (m, 3H), 2.60-2.63 (m, 2H), 2.48-2.41 (m, 2H), 2.30 (s, 3H), 2.01-1.93 (m, 2H), 1.93-1.86 (m, 6H), 1.86-1.74 (m, 1H), 1.68-1.59 (m, 1H), 1.25-1.18 (m, 1H), 0.81-0.74 (m, 6H).

### Example 24: Preparation of Compound 24 and Stereoisomer Thereof

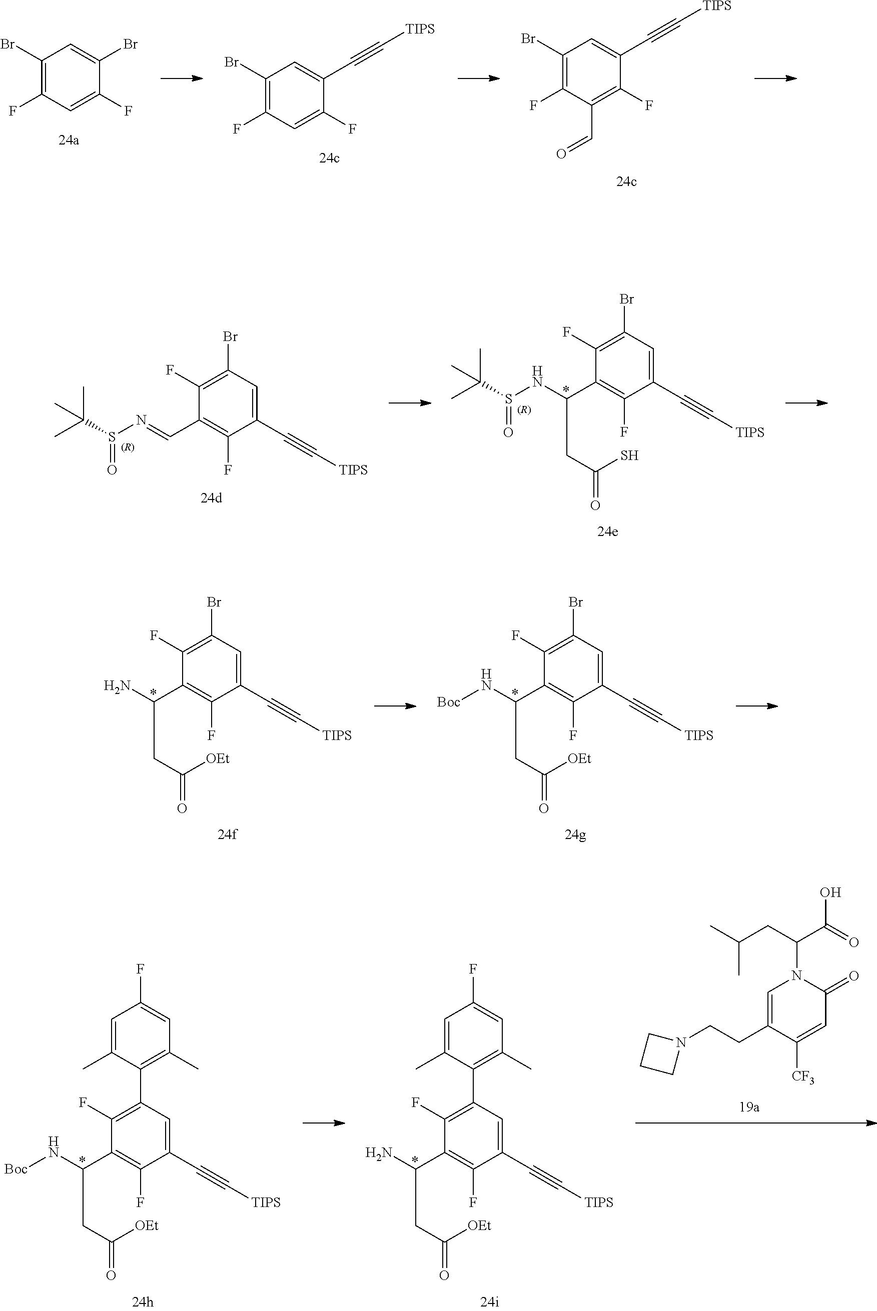

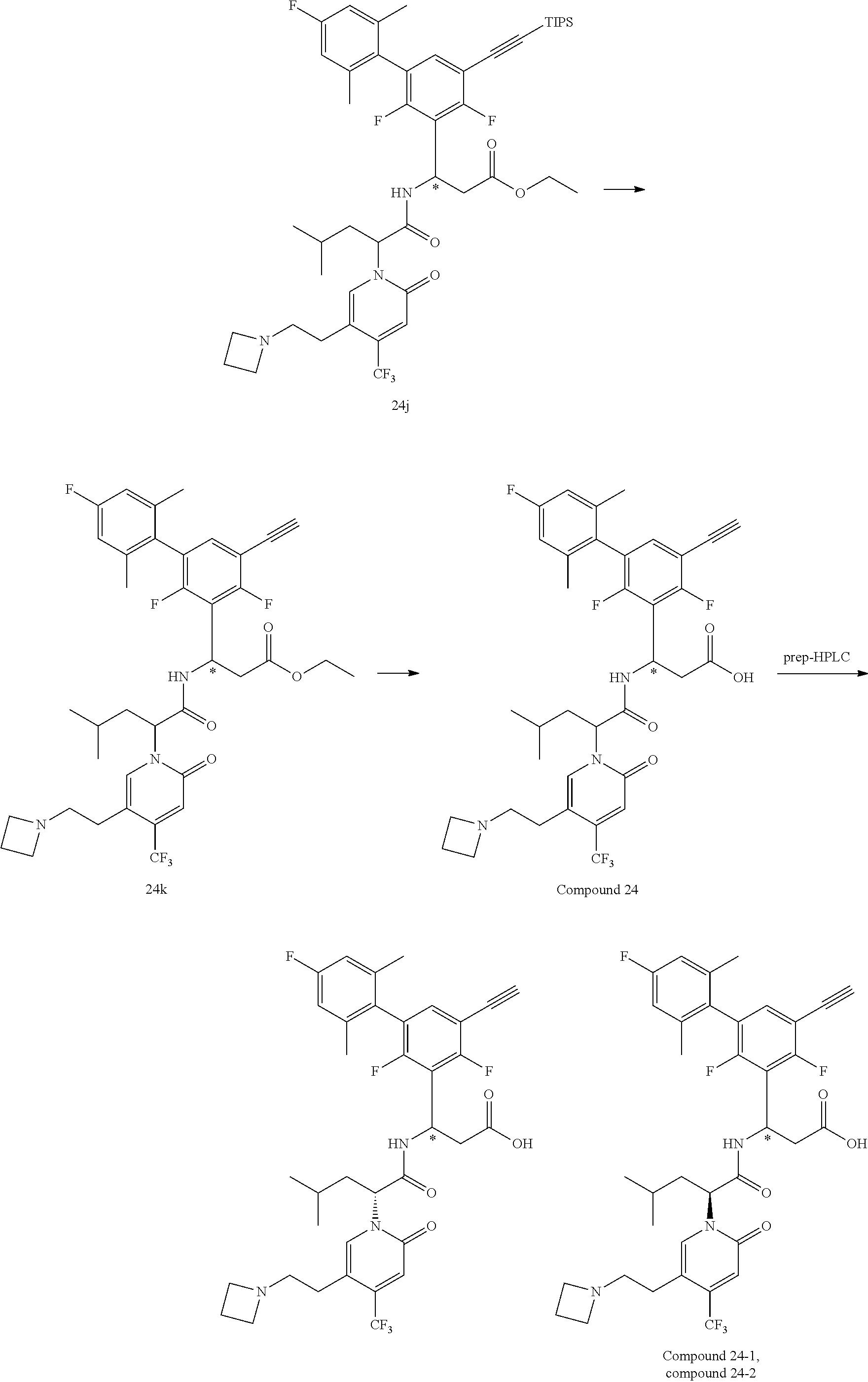

### Step 1: Synthesis of 24b

Under nitrogen-replete conditions, 24a (10.0 g, 37.05 mmol), CuI (0.35 g, 1.85 mmol) and PdCl₂(PPh₃)₂ (1.30 g, 1.85 mmol) were added to a sealed tube, followed by DMF (80 mL), triisopropylsilylethyne (6.08 g, 33.35 mmol) and triethylamine (11.25 g, 111.17 mmol), and the resulting mixture was reacted at 60° C. for 3 h. The reaction mixture was cooled to room temperature, water (250 mL) was added, and the mixture was extracted with ethyl acetate (250 mL×3). The organic phases were combined, washed with water (250 mL) once, washed with saturated brine (250 mL) once, dried over anhydrous sodium sulfate and filtered. The filtrate was concentrated under reduced pressure and the residue was purified by silica gel column chromatography to afford 24b as a colorless liquid (10.3 g, yield: 74.74%).

### Step 2: Synthesis of 24c

24b (9.5 g, 25.53 mmol) was dissolved in THF (150 mL). Under nitrogen protection, lithium diisopropylamide (12.76 mL) (2 mol/L in THF) was added at −78° C. and the mixture was reacted at −78° C. for 1 h. DMF (4 mL) was slowly added dropwise and the resulting mixture was reacted at −78° C. for 1 h. After the dropwise addition was completed, the reaction was quenched at −78° C. with saturated aqueous ammonium chloride solution (150 mL), the reaction mixture was extracted with ethyl acetate (150 mL×3), and the organic layer was washed with saturated brine (200 mL), dried over anhydrous sodium sulfate and filtered. The filtrate was concentrated under reduced pressure and then purified by column chromatography to afford 24c as a colorless liquid (8.16 g, yield: 79.92%).

### Step 3: Synthesis of 24d

24c (8.16 g, 20.39 mmol) and (R)-(+)-tert-butylsulfinamide (2.96 g, 24.47 mmol) were dissolved in THF (150 mL). Under nitrogen protection, tetrabutyl titanate (6.97 g, 30.59 mmol) was added at 0° C. and the resulting mixture was reacted at 45° C. for 3 h. The reaction liquid was cooled to room temperature. Water (150 mL) and ethyl acetate (150 mL) were added and the resulting mixture was filtered. The filter cake was washed with ethyl acetate and the filtrate was extracted with ethyl acetate (150 mL×3). The organic layer was washed with saturated brine (150 mL), dried over anhydrous sodium sulfate and filtered. The filtrate was concentrated under reduced pressure and the residue was purified by column chromatography on a silica gel chromatographic column to afford 24d as a colorless liquid (7.5 g, yield: 73.09%).

### Step 4: Synthesis of 24e

Under nitrogen protection, CuCl (7.38 g, 74.53 mmol) and zinc powder (13.56 g, 208.70 mmol) were added to ultra-dry THF (40 mL) and the mixture was reacted at 60° C. for 2 h and cooled to room temperature. Ethyl bromoacetate (12.44 g, 74.53 mmol) was added and the resulting mixture was reacted at 60° C. for 1 h and cooled to room temperature. Under ice bath conditions, 24d (7.5 g, 14.90 mmol) was added and the reaction mixture was reacted at room temperature for 2 h. The reaction was cooled to room temperature and the reaction liquid was filtered. The filtrate was concentrated under reduced pressure and the residue was purified by column chromatography on a silica gel chromatographic column to afford 24e as a colorless liquid (6.1 g, yield: 69.23%).

### Step 5: Synthesis of Compound 24f

24e (6.1 g, 10.31 mmol) was dissolved in dichloromethane (40 mL), 4 N HCl-1,4-dioxane solution (5 mL) was added, and the resulting mixture was reacted at room temperature for 3 h. The reaction system was concentrated under reduced pressure to afford crude 24f (5.02 g, yield: 100%).

LC-Ms m/z (ESI): 488.3 [M+H]⁺.

### Step 6: Synthesis of 24g

24f (5.02 g, 10.30 mmol) was dissolved in THF (50 mL), di-tert-butyl dicarbonate (4.5 g, 20.60 mmol) and triethylamine (3.12 g, 30.90 mmol) were added, and the resulting mixture was reacted at room temperature for 3 h. The reaction system was concentrated under reduced pressure and the residue was subjected to column chromatography on a silica gel chromatographic column to afford 24g (4.86 g, yield: 80.33%).

LC-Ms m/z (ESI): 588.1 [M+H]⁺.

### Step 7: Synthesis of 24h

Under nitrogen protection, 24g (2 g, 3.4 mmol), (4-fluoro-2,6-dimethoxyphenyl)boronic acid (858 mg, 5.1 mmol), Pd(dppf)Cl₂·DCM (278 mg, 0.34 mmol) and cesium carbonate (2.22 g, 6.81 mmol) were placed into a reaction flask, 1,4-dioxane (15 mL) and water (3 mL) were then added, and the resulting mixture was stirred at 100° C. for 2 h. The reaction mixture was cooled to room temperature and concentrated under reduced pressure. Water (80 mL) was added and the resulting mixture was extracted with ethyl acetate (100 mL×3). The organic phases were combined, dried over anhydrous sodium sulfate and filtered and the filtrate was concentrated under reduced pressure. The residue was purified by chromatographic column to afford 24h as a colorless liquid (520 mg, yield: 24.19%).

LC-Ms m/z (ESI): 632.1 [M+H]⁺.

### Step 8: Synthesis of Compound 24i

24h (260 mg, 0.41 mmol) was dissolved in dichloromethane (4 mL), trifluoroacetic acid (2 mL) was added, and the resulting mixture was stirred at room temperature for 6 h. The reaction mixture was concentrated under reduced pressure to afford 24i.

LC-Ms m/z (ESI): 532.1 [M+H]⁺.

### Step 9: Synthesis of 24j

Under nitrogen protection, 19a (176 mg, 0.49 mmol) and HATU (279 mg, 0.73 mmol) were dissolved in DMF (8 mL), triethylamine (100 mg, 0.98 mmol) was added, and the resulting mixture was reacted at room temperature for 40 min. Crude 24i (260 mg, 0.49 mmol) was added and the mixture was reacted at room temperature overnight. Water (50 mL) was added and the resulting mixture was extracted with ethyl acetate (50 mL×3). The organic phases were combined, washed with water (100 mL) once, washed with saturated brine (100 mL) once, dried over anhydrous sodium sulfate and filtered. The filtrate was concentrated under reduced pressure and the residue was subjected to column chromatography on a silica gel chromatographic column to afford 24j as a colorless liquid (170 mg, yield: 39.81%).

LC-Ms m/z (ESI): 874.3 [M+H]⁺.

### Step 10: Synthesis of 24k

24j (170 mg, 0.19 mmol) was dissolved in DMF (3 mL), cesium fluoride (118 mg, 0.77 mmol) was added, and the resulting mixture was reacted at room temperature for 2 h. The reaction mixture was filtered, the filter cake was washed with ethyl acetate, and the filtrate was concentrated under reduced pressure to afford crude 24k (110 mg, yield: 79.13%).

LC-Ms m/z (ESI): 718.1 [M+H]⁺.

### Step 11: Synthesis of Compounds 24-1 and 24-2

24k (110 mg, 0.15 mmol) was dissolved in anhydrous ethanol (2 mL), a solution of lithium hydroxide monohydrate (25 mg, 0.60 mmol) in water (0.5 mL) was slowly added dropwise, and the resulting mixture was reacted at room temperature for 5 h. Water (6 mL) was added and the resulting mixture was adjusted with 1 N hydrochloric acid aqueous solution to pH=5-6 and extracted with ethyl acetate (15 mL×3). The organic phases were combined, dried over anhydrous sodium sulfate, filtered and concentrated to afford crude compound 24, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid)) to afford the trifluoroacetate of compound 24-1 (14 mg, yield: 13.33%) and trifluoroacetate of compound 24-2 (16 mg, yield: 15.23%).

Trifluoroacetate of compound 24-1: LC-Ms m/z=690.2 [M+H]⁺.

¹H NMR (400 MHz, CDCl₃) δ 11.77 (s, 1H), 7.41 (s, 1H), 6.91 (s, 1H), 6.85 (s, 1H), 6.83 (s, 1H), 6.67 (d, 1H), 6.13-6.04 (m, 1H), 5.77-5.69 (m, 1H), 4.55-4.39 (m, 2H), 3.88-3.73 (m, 1H), 3.65-3.56 (m, 1H), 3.45-3.35 (m, 1H), 3.36-3.27 (m, 1H), 3.10-2.99 (m, 2H), 2.90-2.67 (m, 3H), 2.44-2.33 (m, 1H), 2.04-2.00 (m, 6H), 1.99-1.89 (m, 2H), 1.80-1.71 (m, 1H), 1.46-1.36 (m, 1H), 0.98 (s, 3H), 0.96 (s, 3H).

Trifluoroacetate of compound 24-2: LC-Ms m/z=690.2 [M+H]⁺.

¹H NMR (400 MHz, CDCl₃) δ 11.08 (s, 1H), δ 7.41 (s, 1H), 7.26-7.20 (m, 2H), 6.94 (s, 1H), 6.84 (s, 1H), 6.82 (s, 1H), 6.04-5.93 (m, 1H), 5.56 (t, 1H), 4.55-4.43 (m, 2H), 3.92-3.79 (m, 2H), 3.79-3.67 (m, 2H), 3.30-3.22 (m, 1H), 3.02-2.91 (m, 2H), 2.82-2.65 (m, 3H), 2.46-2.33 (m, 1H), 2.02 (s, 3H), 2.01 (s, 3H), 2.00-1.94 (m, 1H), 1.72-1.64 (m, 1H), 1.44-1.34 (m, 1H), 0.91-0.83 (m, 6H).

### Example 25: Preparation of Compound 25 and Stereoisomer Thereof

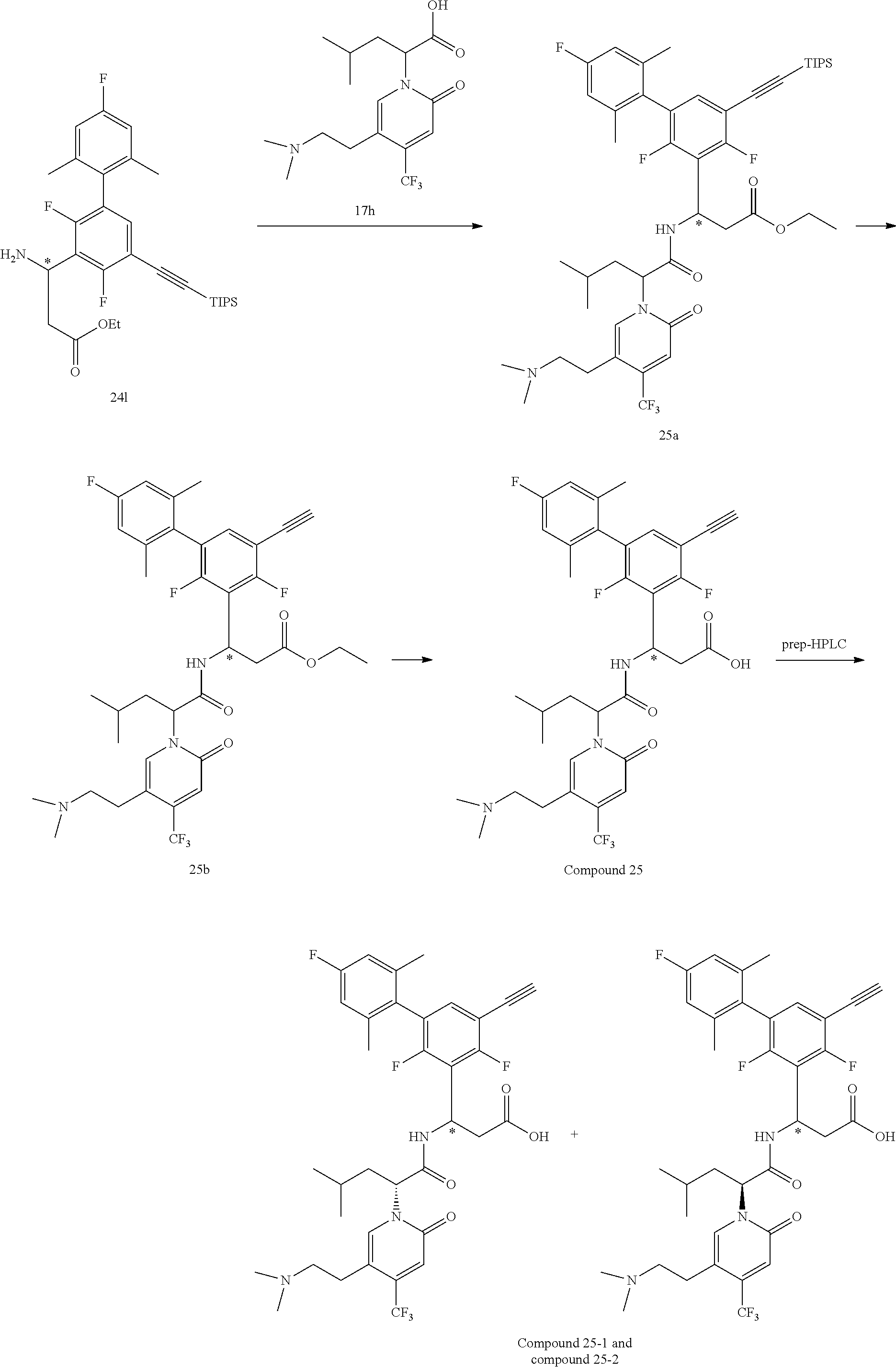

### Step 1: Synthesis of 25a

Under nitrogen protection, intermediate 17h (131 mg, 0.37 mmol) and HATU (214 mg, 0.56 mmol) were dissolved in DMF (8 mL), triethylamine (114 mg, 1.13 mmol) was added, and the resulting mixture was reacted at room temperature for 40 min. Crude 24i (200 mg, 0.37 mmol) was added and the mixture was reacted at room temperature overnight. Water (50 mL) was added and the resulting mixture was extracted with ethyl acetate (5 mL×3). The organic phases were combined, washed with water (100 mL) once, washed with saturated NaCl aqueous solution (100 mL) once, dried over anhydrous sodium sulfate and filtered. The filtrate was concentrated under reduced pressure and the residue was subjected to column chromatography on a silica gel chromatographic column to afford 25a as a colorless liquid (135 mg, yield: 41.66%).

LC-Ms m/z=862.3 [M+H]⁺.

### Step 2: Synthesis of 25b

25a (135 mg, 0.15 mmol) was dissolved in DMF (3 mL), cesium fluoride (95 mg, 0.62 mmol) was added, and the resulting mixture was reacted at room temperature for 2 h. The reaction mixture was filtered, the filter cake was washed with ethyl acetate, and the filtrate was concentrated under reduced pressure to afford crude 25b (80 mg, yield: 72.72%).

LC-Ms m/z (ESI): 706.1 [M+H]⁺.

### Step 3: Synthesis of Compounds 25-1 and 25-2

25b (80 mg, 0.11 mmol) was dissolved in anhydrous ethanol (2 mL), a solution of lithium hydroxide monohydrate (19 mg, 0.45 mmol) in water (0.5 mL) was slowly added dropwise, and the resulting mixture was reacted at room temperature for 5 h. Water (6 mL) was added and the resulting mixture was adjusted with 1 N hydrochloric acid aqueous solution to pH=5-6 and extracted with ethyl acetate (15 mL×3). The organic phases were combined, dried over anhydrous sodium sulfate, filtered and concentrated to afford crude compound 25, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid)) to afford the trifluoroacetate of compound 25-1 (6 mg, yield: 7.89%) and trifluoroacetate of compound 25-2 (10 mg, yield: 13.15%).

Trifluoroacetate of compound 25-1: LC-Ms m/z (ESI): 678.1 [M+H]⁺.

¹H NMR (400 MHz, CDCl₃) δ 8.12 (s, 1H), 7.42 (s, 1H), 7.15 (t, 1H), 6.94 (s, 1H), 6.82 (s, 1H), 6.80 (s, 1H), 5.68-5.60 (m 2H), 3.20-3.12 (m, 2H), 2.96-2.83 (m, 2H), 2.68 (d, 2H), 2.59 (s, 6H), 2.03 (s, 3H), 2.00 (s, 3H), 1.97-1.91 (m, 1H), 1.82-1.71 (m, 2H), 1.47-1.36 (m, 1H), 0.92 (s, 3H), 0.91 (s, 3H).

Trifluoroacetate of compound 25-2: LC-Ms m/z (ESI): 678.1 [M+H]⁺.

¹H NMR (400 MHz, CDCl₃) δ 7.22 (s, 1H), 7.22-7.16 (m, 2H), 7.01 (s, 1H), 6.84-6.79 (m, 2H), 6.03-5.94 (m, 1H), 5.66-5.58 (m, 1H), 3.10-2.99 (m, 1H), 3.00-2.88 (m, 1H), 2.78-2.58 (m, 4H), 2.46 (s, 6H), 2.43-2.37 (m, 1H), 2.11-2.03 (m, 1H), 2.02 (s, 3H), 2.01 (s, 3H), 1.78-1.69 (m, 1H), 1.48-1.40 (m, 1H), 0.94-0.85 (m, 6H).

### Example 26: Preparation of Compound 26 and Stereoisomer Thereof

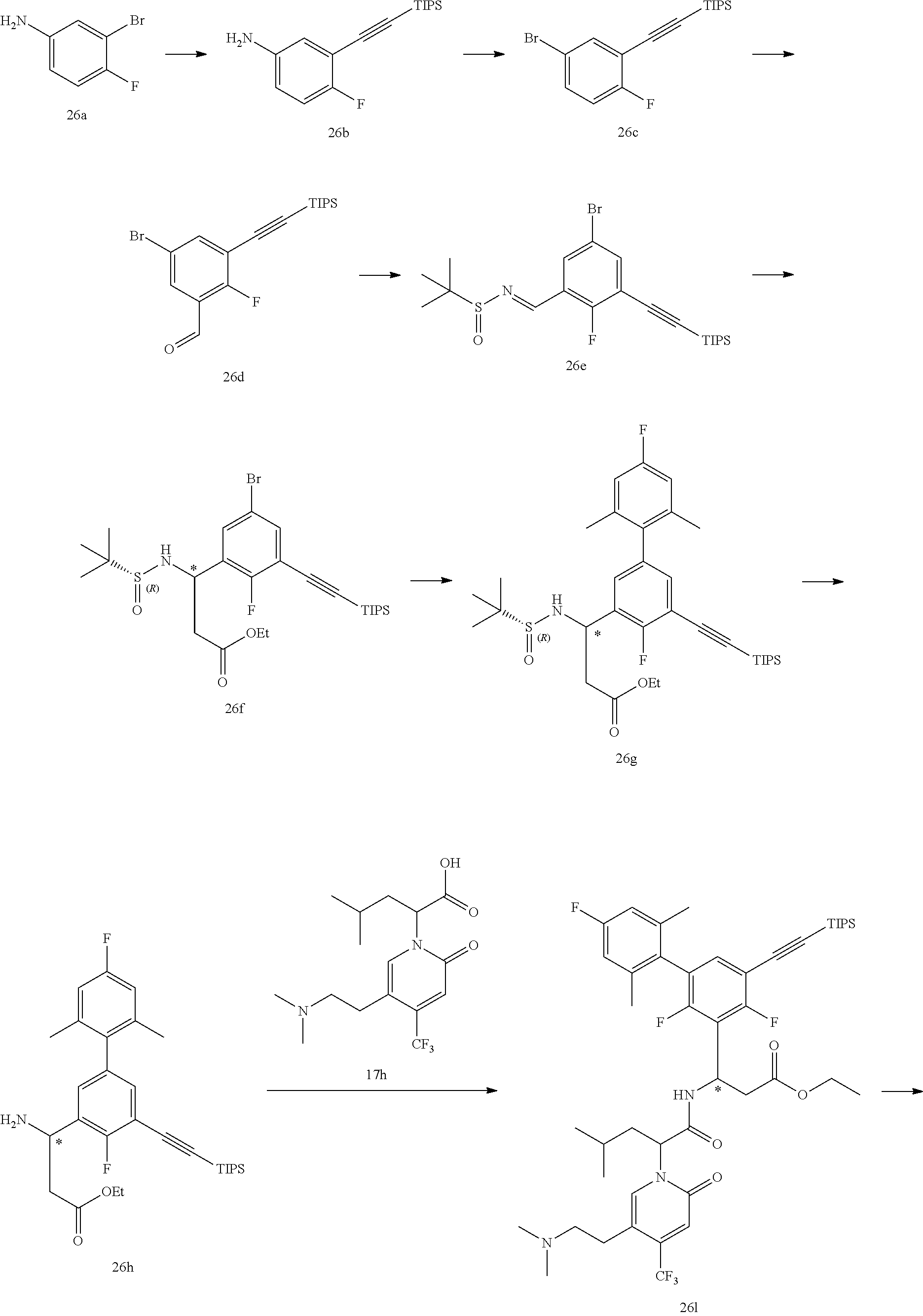

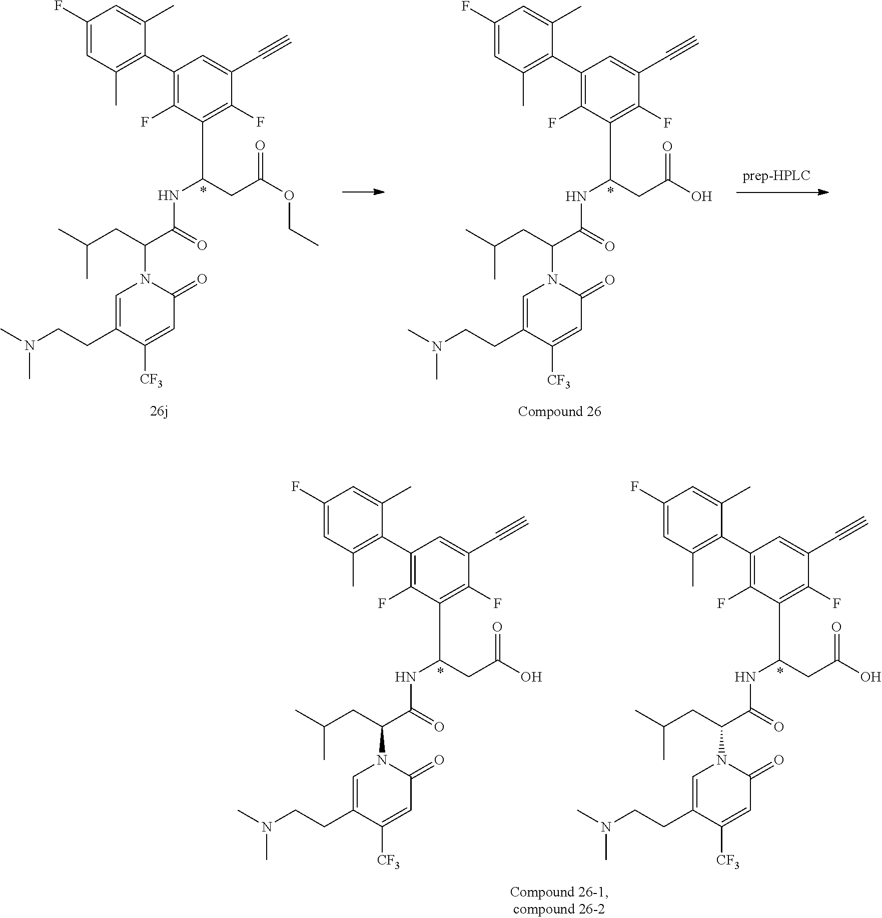 [SMILES: C#Cc1cc(-c2c(C)cc(F)cc2C)c(F)c(C(CC(=O)OCC)NC(=O)C(CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)C(CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)[C@H](CC(C)C)n2cc(CCN(C)C)c(C(F)(F)F)cc2=O)c1F ; C#Cc1cc(-c2c(C)cc(F)cc2C)c(F)c(C(CC(=O)O)NC(=O)[C@@H](CC(C)C)n2cc(CCN(C)C)c(C(F)(F)F)cc2=O)c1F]

### Step 1: Synthesis of 26b

Under nitrogen-replete conditions, 26a (5.0 g, 26.33 mmol), CuI (0.5 g, 2.63 mmol) and PdCl₂ (PPh₃)₂ (1.85 g, 2.63 mmol) were added to a sealed tube, followed by DMF (50 mL), triisopropylsilylethyne (7.2 g, 39.49 mmol) and triethylamine (7.9 g, 78.99 mmol), and the resulting mixture was reacted at 60° C. for 3 h. The reaction mixture was cooled to room temperature and water (200 mL) was added. The resulting mixture was extracted with ethyl acetate (200 mL×3) and the organic phases were combined, dried over anhydrous sodium sulfate, filtered and concentrated under reduced pressure. The residue was purified by silica gel column chromatography to afford 26b (6.0 g, yield: 78.18%).

### Step 2: Synthesis of 26c

26b (6.0 g, 20.58 mmol) and cuprous bromide (7.4 g, 51.45 mmol) were dissolved in acetonitrile (100 mL) and water (20 mL). At 0° C., p-toluenesulfonic acid (9.21 g, 53.51 mmol) was added and the resulting mixture was continuously stirred for 1 h. A solution of sodium nitrite (2.1 g, 30.87 mmol) in water (6 L) was added dropwise. After the dropwise addition was completed, the reaction mixture was extracted with ethyl acetate (200 mL×3) and the organic layer was washed with brine, dried over anhydrous sodium sulfate, filtered and concentrated under reduced pressure. The residue was purified by column chromatography to afford 26c (3.3 g, yield: 45.12%).

### Step 3: Synthesis of 26d

26c (1.5 g, 4.22 mmol) was dissolved in THF (25 mL). Under nitrogen protection, lithium diisopropylamide (7.5 mL) was added at −78° C. and the resulting mixture was continuously reacted for 1 h. DMF (2 mL) was slowly added dropwise and the mixture was reacted at −78° C. for 1 h. The reaction was quenched with saturated aqueous ammonium chloride solution (150 mL) and the reaction mixture was extracted with ethyl acetate (100×3). The organic layer was washed with brine, dried over anhydrous sodium sulfate, filtered and concentrated under reduced pressure. The residue was purified by column chromatography to afford 26d (1.34 g, yield: 82.83%).

### Step 4: Synthesis of 26e

26d (1.34 g, 3.50 mmol) and (R)-(+)-tert-butylsulfinamide (509.0 mg, 4.20 mmol) were dissolved in THF (15 mL). Under nitrogen protection, tetrabutyl titanate (1.79 mL) was added at 0° C. and the resulting mixture was reacted overnight. The reaction mixture was filtered, the filter cake was washed with ethyl acetate, and the filtrate was extracted with ethyl acetate. The organic phase was washed with water, dried over anhydrous sodium sulfate, filtered and concentrated under reduced pressure. The residue was purified by chromatographic column to afford 26e (1.2 g, yield: 70.47%).

### Step 5: Synthesis of 26f

Under nitrogen protection, CuCl (914.8 mg, 9.24 mmol) and zinc powder (2.8 g, 43.12 mmol) were added to THF (20 mL) and the resulting mixture was reacted at 60° C. for 2 h and cooled to room temperature. Ethyl bromoacetate (2.6 g, 15.40 mmol) was added and the mixture was reacted at 60° C. for 1 h and cooled to room temperature. Under ice bath, compound 26e (1.5 g, 3.08 mmol) was added and the mixture was reacted for 2 h. The reaction system was extracted with ethyl acetate and the organic phase was washed with water, dried over anhydrous sodium sulfate, filtered and concentrated under reduced pressure. The residue was purified by chromatography to afford 26f (1.2 g, yield: 67.80%).

### Step 6: Synthesis of 26g

Under nitrogen protection, 26f (700.0 mg, 1.22 mmol), (4-fluoro-2,6-dimethoxyphenyl)boronic acid (245.9 mg, 1.46 mmol), Pd(dppf)Cl₂ (199.2 mg, 0.24 mmol) and cesium carbonate (1.2 g, 3.66 mmol) were dissolved in 1,4-dioxane (7 mL) and water (1 mL) and the resulting mixture was stirred at 100° C. for 2 h. The reaction system was cooled to room temperature and the solvent was removed under reduced pressure. Water (80 mL) was added and the resulting mixture was extracted with ethyl acetate (100 mL×3). The organic phases were combined, dried over anhydrous sodium sulfate and filtered and the filtrate was concentrated under reduced pressure and then purified by chromatographic column to afford 26g (350.0 mg, yield: 46.43%).

### Step 7: Synthesis of 26h

26g (350.0 mg, 0.57 mmol) was dissolved in dichloromethane (4 mL), 4 N hydrogen chloride-1,4-dioxane solution (4 mL) was added, and the resulting mixture was stirred at room temperature for 6 h. The reaction mixture was concentrated under reduced pressure to afford 26h.

LC-Ms m/z (ESI): 514.8 [M+H]⁺.

### Step 8: Synthesis of 26i

Under nitrogen protection, 17h (284.3 mg, 0.82 mmol) and HATU (517.1 mg, 1.36 mmol) were dissolved in DMF (4 mL), triethylamine (351.5 mg, 2.72 mmol) was added, and the resulting mixture was reacted at room temperature for 40 min. Crude 26h (350.0 mg, 0.68 mmol) was added and the mixture was reacted at room temperature overnight. Water (80 mL) was added and the resulting mixture was extracted with ethyl acetate (100 mL×3). The organic phases were combined, dried over anhydrous sodium sulfate and filtered and the filtrate was concentrated under reduced pressure and then purified by chromatographic column to afford 26i (330.0 mg, yield: 57.49%).

LC-Ms m/z (ESI): 844.4 [M+H]⁺.

### Step 9: Synthesis of 26j

26i (330.0 mg, 0.39 mmol) was dissolved in DMF (3 mL), cesium fluoride (177.7 mg, 1.17 mmol) was added, and the resulting mixture was reacted at room temperature for 2 h. The reaction mixture was filtered, the filter cake was washed with ethyl acetate, and the filtrate was concentrated under reduced pressure to afford 26j.

LC-Ms m/z (ESI): 688.3 [M+H]⁺.

### Step 10: Synthesis of Compounds 26-1 and 26-2

26j (250.0 mg, 0.36 mmol) was dissolved in THF (2 mL), a solution of lithium hydroxide (17.0 mg, 0.71 mmol) in water (2 mL) was slowly added dropwise, and the resulting mixture was reacted at room temperature for 5 h. Water (6 mL) was added and the resulting mixture was adjusted with 1 N hydrochloric acid aqueous solution to pH=5-6 and extracted with ethyl acetate (15 mL×3). The organic phases were combined, dried over anhydrous sodium sulfate, filtered and concentrated to afford crude compound 26, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 26-1 (30.0 mg, yield: 10.11%) and compound 26-2 (50.0 mg, yield: 20.21%).

Compound 26-1:

LC-Ms m/z=660.3 [M+H]⁺.

¹H NMR (400 MHz, DMSO-d₆) δ 9.07 (d, 1H), 7.78 (s, 1H), 7.19-7.15 (m, 1H), 7.14-7.09 (m, 1H), 7.00-6.94 (m, 1H), 6.94-6.88 (m, 1H), 6.61 (s, 1H), 5.54-5.47 (m, 1H), 5.44-5.35 (m, 1H), 4.49 (s, 1H), 2.70 (d, 2H), 2.62-2.52 (m, 2H), 2.41-2.29 (m, 2H), 2.17 (s, 6H), 1.99-1.88 (m, 4H), 1.82-1.71 (m, 4H), 1.40-1.28 (m, 1H), 0.88 (d, 3H), 0.82 (d, 3H).

Compound 26-2:

LC-Ms m/z=660.3 [M+H]⁺.

¹H NMR (400 MHz, DMSO-d₆) δ 9.13 (d, 1H), 7.80 (s, 1H), 7.25-7.21 (m, 1H), 7.20-7.16 (m, 1H), 7.02 (d, 1H), 6.99 (d, 1H), 6.74 (s, 1H), 5.60-5.53 (m, 1H), 5.47-5.40 (m 1H), 4.53 (s, 1H), 2.73-2.66 (d, 2H), 2.64-2.55 (m, 2H), 2.46-2.39 (m, 2H), 2.20 (s, 6H), 1.97 (d, 6H), 1.81-1.71 (m, 1H), 1.63-1.55 (m, 1H), 1.25-1.11 (m, 1H), 0.79-0.73 (m, 6H).

### Example 27: Preparation of Compound 27 and Stereoisomer Thereof

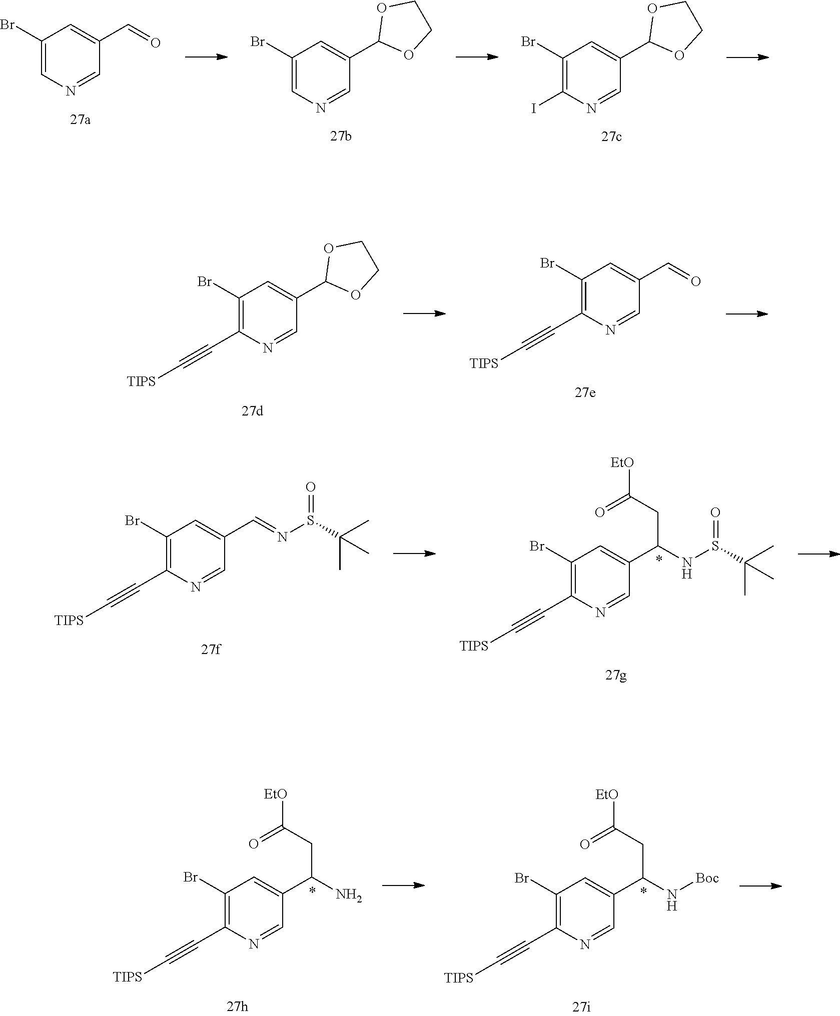

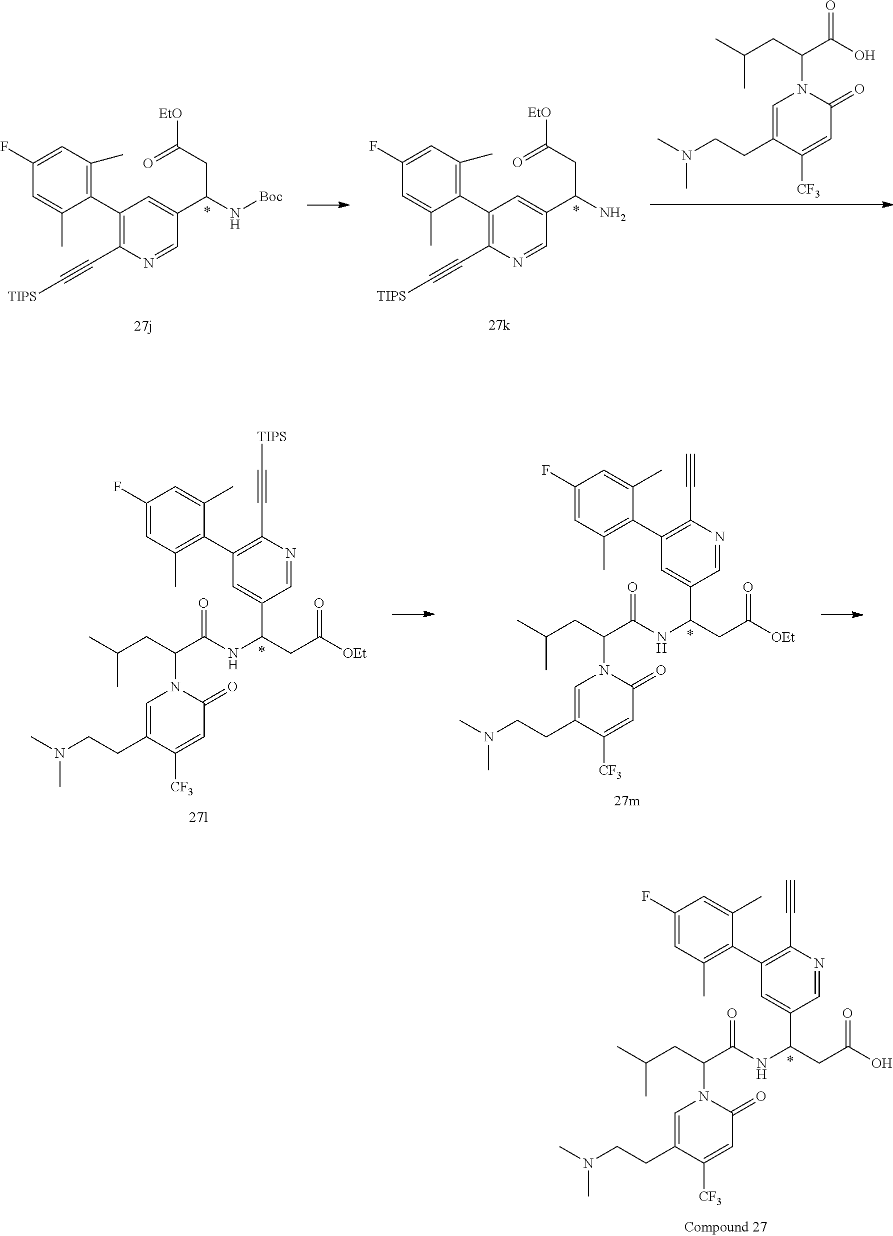

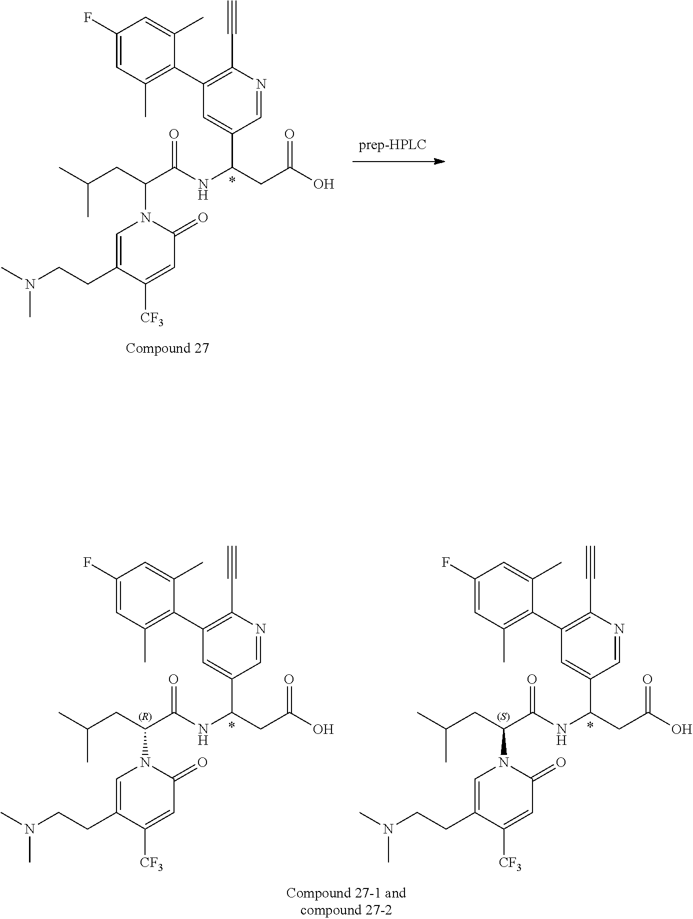

### Step 1: Synthesis of 27b

27a (10.0 g, 53.8 mmol) was dissolved in toluene (170 mL), and ethylene glycol (33.4 g, 573.6 mmol) and p-toluenesulfonic acid monohydrate (930.0 mg, 5.4 mmol) were successively added. Under nitrogen protection, the mixture was stirred at 120° C. for 3 h. The reaction liquid was cooled to room temperature, then poured into saturated sodium bicarbonate solution (72 mL), and allowed to stand for layering. The organic phase was collected and the aqueous phase was extracted with ethyl acetate (30 mL×2). The organic phases were combined and washed with saturated sodium chloride. The organic phase was collected, dried over anhydrous sodium sulfate, filtered and concentrated to afford crude 27b (10.5 g, yield: 85.4%).

LC-Ms m/z (ESI): 230.0 [M+H]⁺.

### Step 2: Synthesis of 27c

27b (10.5 g, 45.9 mmol) was dissolved in anhydrous THF (300 mL). Under nitrogen protection, the mixture was stirred at −40° C. for 30 min and magnesium dichloride(2,2,6,6-tetramethylpiperidine)lithium salt (16.7 g, 68.9 mmol) was slowly added dropwise and stirred at −40° C. for 5 h. Iodine (17.5 g, 68.9 mmol) was added in portions and stirred at −40° C. for 30 min. The resulting mixture was naturally warmed to room temperature and reacted for 18 h. Saturated sodium thiosulfate solution was added and the reaction mixture was allowed to stand for layering. The aqueous phase was extracted with ethyl acetate (50 mL×3), and the organic phases were combined, dried over anhydrous sodium sulfate, filtered and concentrated and the residue was subjected to silica gel column chromatography to afford 27c (11.3 g, yield: 69.3%).

LC-Ms m/z (ESI): 355.9 [M+H]⁺.

### Step 3: Synthesis of 27d

27c (11.3 g, 31.8 mmol) was dissolved in THF (150 mL). Ethynyltriisopropylsilane (5.8 g, 31.8 mmol), Pd(PPh₃)₄ (693 mg, 0.6 mmol), CuI (304.7 mg, 1.6 mmol) and DIPEA (17.2 g, 133.6 mmol) were successively added. Under nitrogen protection, the mixture was stirred at 0° C. for 1 h and reacted at room temperature for 18 h. The reaction mixture was filtered, water (200 mL) was added to the filtrate, and the mixture was allowed to stand for layering. The aqueous phase was extracted with ethyl acetate (50 mL×3), and the organic phases were combined, dried over anhydrous sodium sulfate, filtered and concentrated to afford a crude, which was purified by silica gel column to afford 27d (8.2 g, yield: 63.1%).

LC-Ms m/z (ESI): 411.4 [M+H]⁺.

### Step 4: Synthesis of 27e

27d (8.2 g, 20.0 mmol) was dissolved in THF (190 mL) and 3 M hydrochloric acid aqueous solution (96 mL) was added. Under nitrogen protection, the mixture was stirred at room temperature for 18 h. The reaction liquid was allowed to stand for layering, the organic phase was collected, and the aqueous phase was extracted with ethyl acetate (50 mL×2). The organic phases were combined and washed with saturated sodium bicarbonate solution. The organic phase was collected, dried over anhydrous sodium sulfate, filtered and concentrated to afford crude 27e.

### Step 5: Synthesis of 27f

27e (6.8 g, 17.7 mmol) was dissolved in THF (100 mL). Under ice bath conditions, (R)-(+)-tert-butylsulfinamide (2.6 g, 21.3 mmol) and tetraethyl titanate (10.1 g, 44.3 mmol) were successively added. Under nitrogen protection, the mixture was stirred at 55° C. for 3 h. The reaction mixture was cooled to room temperature, followed by the addition of ice water (100 mL). The mixture was filtered and the filter cake was washed with ethyl acetate (20 mL×2). The filtrate was collected and allowed to stand for layer separation. The organic phase was collected and the aqueous phase was extracted with ethyl acetate (30 mL×3). The organic phases were combined, dried over anhydrous sodium sulfate, filtered and concentrated and the residue was subjected to silica gel column chromatography to afford 27f (7.9 g, yield: 95.2%).

LC-Ms m/z (ESI): 470.5 [M+H]⁺.

### Step 6: Synthesis of 27g

Zinc powder (15.3 g, 235.2 mmol) was added to anhydrous THF (180 mL), CuCl (5.0 g, 50.4 mmol) was added, and the mixture was subjected to nitrogen replacement three times and stirred at 60° C. for 1 h. At 0° C., a solution of ethyl bromoacetate (14.0 g, 84.0 mmol) in THF (10 mL) was added dropwise and the mixture was stirred at 60° C. for 1 h. At 0° C., a solution of 27f (7.9 g, 16.8 mmol) in THF (10 mL) was added dropwise and the resulting mixture was stirred at 0° C. for 3 h. The reaction mixture was filtered and the filtrate was quenched with 1 N HCl, and extracted with ethyl acetate (50 mL×3). The organic phases were combined, dried over anhydrous sodium sulfate, filtered and concentrated and the residue was purified by silica gel column to afford 27g (8.1 g, yield: 86.2%).

LC-Ms m/z (ESI): 558.6 [M+H]⁺.

### Step 7: Synthesis of 27h

27g (8.1 g, 14.5 mmol) was dissolved in THF (12 mL), a 4 M hydrogen chloride 1,4-dioxane solution (12 mL) was added at room temperature, and the mixture was stirred at room temperature for 2 h. The reaction liquid was concentrated under reduced pressure to afford crude 27h.

LC-Ms m/z (ESI): 454.5[M+H]⁺.

### Step 8: Synthesis of 27i

27h (8 g, 17.6 mmol) was dissolved in THF (15 mL) and water (20 mL), sodium carbonate (3.7 g, 35.2 mmol) was added, a solution of di-tert-butyl dicarbonate (4.2 g, 19.4 mmol) in THF (5 mL) was added dropwise, and the mixture was stirred at room temperature for 3 h. The reaction mixture was extracted with ethyl acetate (20 mL×2), and the organic phases were combined, dried over anhydrous sodium sulfate, filtered and concentrated and the residue was subjected to silica gel column chromatography to afford 27i (7.4 g, yield: 76.3%).

LC-Ms m/z (ESI): 554.6[M+H]⁺.

### Step 9: Synthesis of 27j

27i (2 g, 3.6 mmol) was dissolved in 1,4-dioxane (20 mL) and water (2 mL), and 4-fluoro-2,6-dimethylphenylboronic acid (726 mg, 4.3 mmol), Pd(dppf)Cl₂ (294 mg, 0.36 mmol) and cesium carbonate (3.5 g, 10.8 mmol) were successively added. Under nitrogen protection, the mixture was stirred at 90° C. for 6 h. The reaction liquid was cooled to room temperature and filtered, the filtrate was concentrated under reduced pressure, and ethyl acetate (50 mL) and water (50 mL) were added for layering and extraction. The organic phase was collected and the aqueous phase was extracted with ethyl acetate (50 mL×2). The organic phases were combined, dried over anhydrous sodium sulfate, filtered and concentrated under reduced pressure and the residue was subjected to silica gel column chromatography to afford 27j (1.5 g, yield: 71.4%).

LC-Ms m/z (ESI): 597.8[M+H]⁺.

### Step 10: Synthesis of 27k

27j (1.5 g, 2.5 mmol) was dissolved in dichloromethane (10 mL), trifluoroacetic acid (2 mL) was added, and the resulting mixture was stirred at room temperature for 2 h. The reaction liquid was concentrated under reduced pressure to afford crude 27k.

LC-Ms m/z (ESI): 497.7[M+H]⁺.

### Step 11: Synthesis of 27l

17h (417 mg, 1.2 mmol) was dissolved in DMF (10 mL), 27k (600 mg, 1.2 mmol), HATU (912 mg, 2.4 mmol) and DIPEA (624 mg, 4.8 mmol) were added, and the mixture was reacted at room temperature for 1 h. Water (50 mL) was added and the resulting mixture was extracted with ethyl acetate (15 mL×3). The organic phases were combined and washed with saturated brine. The organic phase was collected, dried over anhydrous sodium sulfate, filtered and concentrated to afford a crude, which was subjected to silica gel column chromatography to afford 27l (500 mg, yield: 50.4%).

LC-Ms m/z (ESI): 828.0[M+H]⁺.

### Step 12: Synthesis of 27m

27l (500 mg, 0.6 mmol) was dissolved in methanol (3 mL), cesium fluoride (360 mg, 2.39 mmol) was added, and the mixture was reacted at room temperature for 18 h. Water (10 mL) was added and the resulting mixture was extracted with ethyl acetate (10 mL×3). The organic phases were combined, dried over anhydrous sodium sulfate, filtered and concentrated to afford crude 27m (300 mg).

LC-Ms m/z (ESI): 671.7[M+H]⁺.

### Step 13: Synthesis of Compounds 27-1 and 27-2

27m (300 mg, 0.45 mmol) was dissolved in THF (5 mL) and water (1.5 mL), lithium hydroxide monohydrate (33 mg, 1.37 mmol) was added, and the resulting mixture was reacted at room temperature for 6 h. The reaction mixture was adjusted with 1 N hydrochloric acid to pH 5-6 and concentrated under reduced pressure to afford crude compound 27, which was directly subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 27-1 (34.98 mg, retention time=5.183 min, yield: 12.1%) and compound 27-2 (39 mg, retention time=5.237 min, yield: 13.5%).

Compound 27-1: LC-Ms m/z (ESI): 643.7[M+H]⁺.

¹H NMR (400 MHz, DMSO-d₆) δ 9.09 (d, 1H), 8.50 (d, 1H), 7.80 (s, 1H), 7.48 (d, 1H), 7.03-6.93 (m, 2H), 6.65 (s, 1H), 5.54-5.46 (m, 1H), 5.26-5.15 (m, 1H), 4.01 (s, 1H), 2.86-2.69 (m, 2H), 2.66-2.53 (m, 2H), 2.44-2.29 (m, 2H), 2.24-2.21 (m, 6H), 2.00-1.85 (m, 4H), 1.82-1.71 (m, 4H), 1.42-1.27 (m, 1H), 0.95-0.73 (m, 6H).

Compound 27-2: LC-Ms m/z (ESI): 643.7[M+H]⁺.

¹H NMR (400 MHz, DMSO-d₆) δ 9.14 (d, 1H), 8.54 (d, 1H), 7.81 (s, 1H), 7.54 (d, 1H), 7.03 (d, 2H), 6.75 (s, 1H), 5.61-5.51 (m, 1H), 5.25-5.13 (m, 1H), 4.04 (s, 1H), 2.77 (d, 2H), 2.66-2.53 (m, 2H), 2.44-2.29 (m, 2H), 2.19 (s, 6H), 1.92 (s, 6H), 1.84-1.75 (m, 1H), 1.69-1.58 (m, 1H), 1.26-1.12 (m, 1H), 0.83-0.72 (m, 6H).

### Example 28: Preparation of Compound 28 and Stereoisomer Thereof

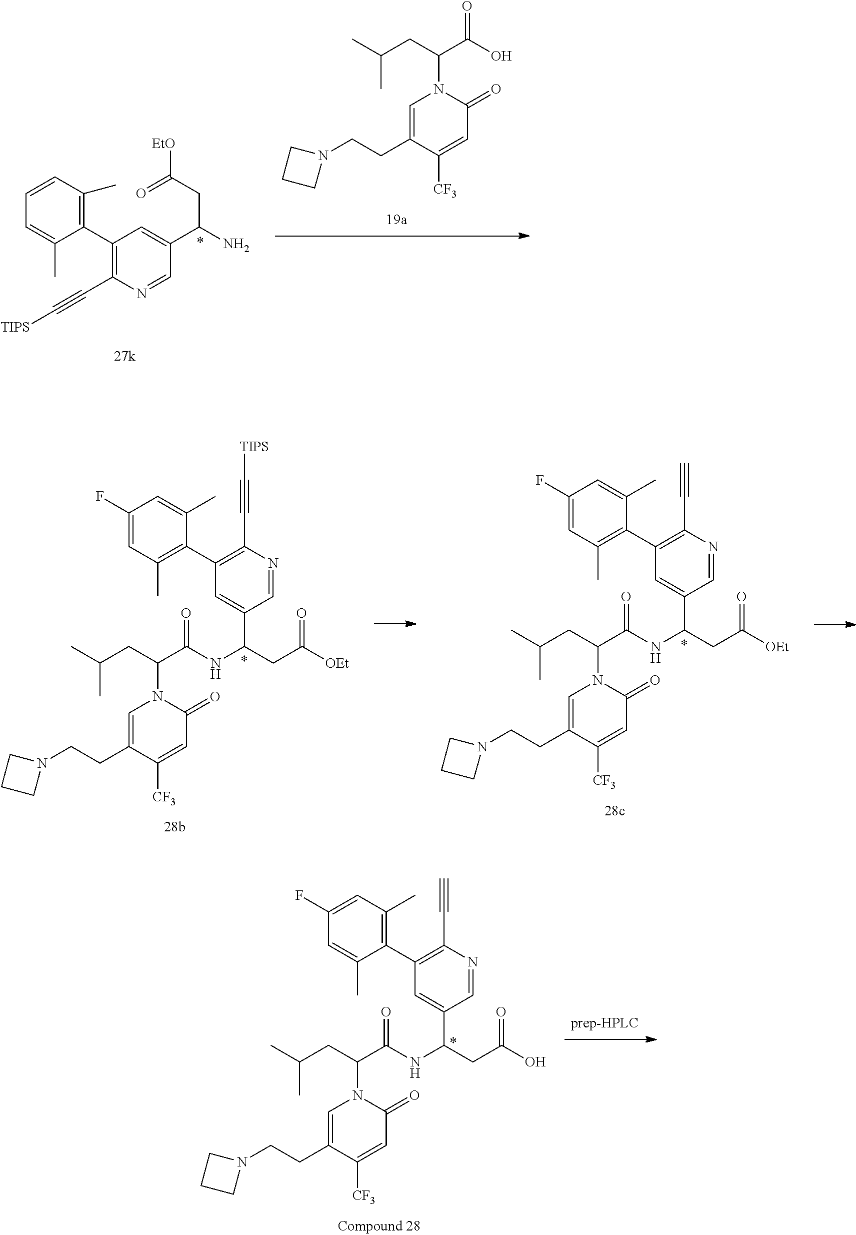

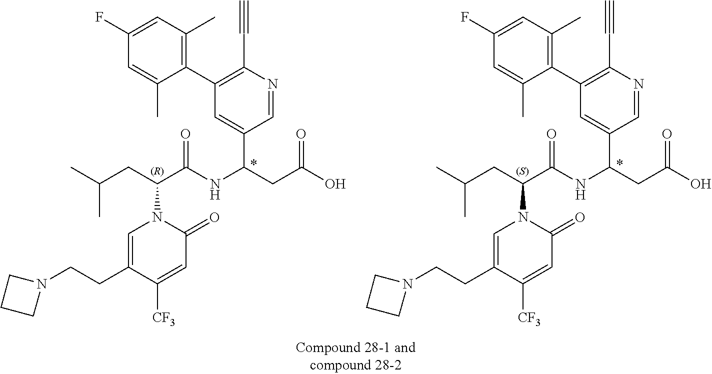

### Step 1: Synthesis of 28b

19a (220 mg, 0.60 mmol) was dissolved in DMF (10 mL), 27k (300 mg, 0.60 mmol), HATU (460 mg, 1.21 mmol) and DIPEA (310 mg, 2.4 mmol) were successively added, and the mixture was reacted at room temperature for 1 h. Water (50 mL) was added and the resulting mixture was extracted with ethyl acetate (15 mL×3). The organic phases were combined and washed with saturated brine. The organic phase was collected, dried over anhydrous sodium sulfate, filtered and concentrated to afford a crude, which was separated and purified by silica gel column chromatography to afford 28b (423 mg, yield: 84%).

LC-Ms m/z (ESI): 840.1[M+H]⁺.

### Step 2: Synthesis of Compound 28c

28b (423 mg, 0.50 mmol) was dissolved in methanol (3 mL), cesium fluoride (300 mg, 2.0 mmol) was added, and the mixture was reacted at room temperature for 18 h. Water (10 mL) was added and the resulting mixture was extracted with ethyl acetate (10 mL×3). The organic phases were combined, dried over anhydrous sodium sulfate, filtered and concentrated to afford crude 28c (289 mg).

LC-Ms m/z (ESI): 683.7[M+H]⁺.

### Step 3: Synthesis of Compounds 28-1 and 28-2

28c (289 mg, 0.42 mmol) was dissolved in THF (5 mL) and water (1.5 mL), lithium hydroxide monohydrate (30 mg, 1.27 mmol) was added, and the resulting mixture was reacted at room temperature for 6 h. The reaction mixture was adjusted with 1 N hydrochloric acid to pH 5-6, and concentrated under reduced pressure to afford crude compound 28, which was separated and purified by prep-HPLC (composition of mobile phases: mobile phase A: acetonitrile, and mobile phase B: water (containing 0.1% ammonium acetate)) to afford compound 28-1 (19 mg, retention time=3.770 min, yield: 6.9%) and compound 28-2 (17 mg, retention time=3.859 min, yield: 6.2%).

Compound 28-1: LC-Ms m/z (ESI): 655.7[M+H]⁺.

¹H NMR (400 MHz, DMSO-d₆) δ 9.21 (d, 1H), 8.50 (d, 1H), 7.79 (s, 1H), 7.47 (d, 1H), 7.03-6.93 (m, 2H), 6.66 (s, 1H), 5.50-5.41 (m, 1H), 5.22-5.12 (m, 1H), 4.00 (s, 1H), 3.30-3.15 (m, 4H), 2.76-2.70 (m, 2H), 2.62-2.55 (m, 2H), 2.45-2.36 (m, 2H), 2.03-1.92 (m, 3H), 1.89 (s, 3H), 1.85-1.76 (m, 4H), 1.32 (s, 1H), 0.90 (d, 3H), 0.84 (d, 3H).

Compound 28-2: LC-Ms m/z (ESI): 655.7[M+H]⁺.

¹H NMR (400 MHz, DMSO-d₆) δ 9.11 (d, 1H), 8.54 (d, 1H), 7.74 (s, 1H), 7.53 (d, 1H), 7.03 (d, 2H), 6.75 (s, 1H), 5.54-5.46 (m, 1H), 5.25-5.17 (m, 1H), 4.03 (s, 1H), 3.32-3.15 (m, 4H), 2.74-2.57 (m, 4H), 2.55-2.45 (m, 2H), 2.06-1.95 (m, 2H), 1.92 (s, 6H), 1.83-1.65 (m, 2H), 1.30-1.17 (m, 1H), 0.86-0.73 (m, 6H).

### Example 29: Preparation of Compound 29 and Stereoisomer Thereof

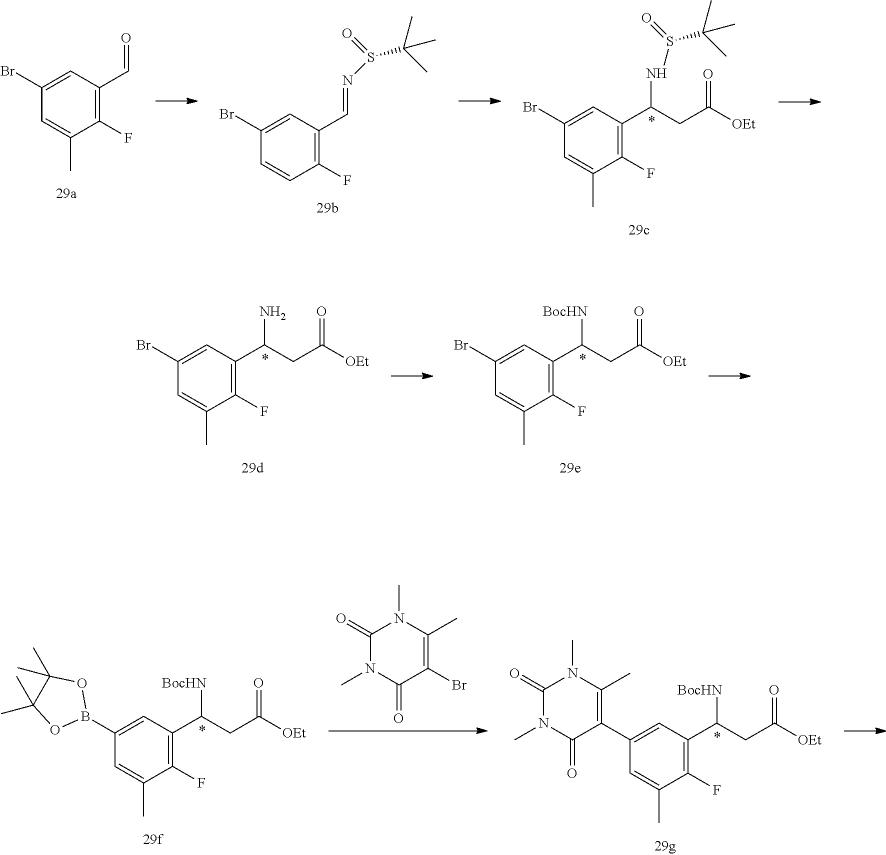

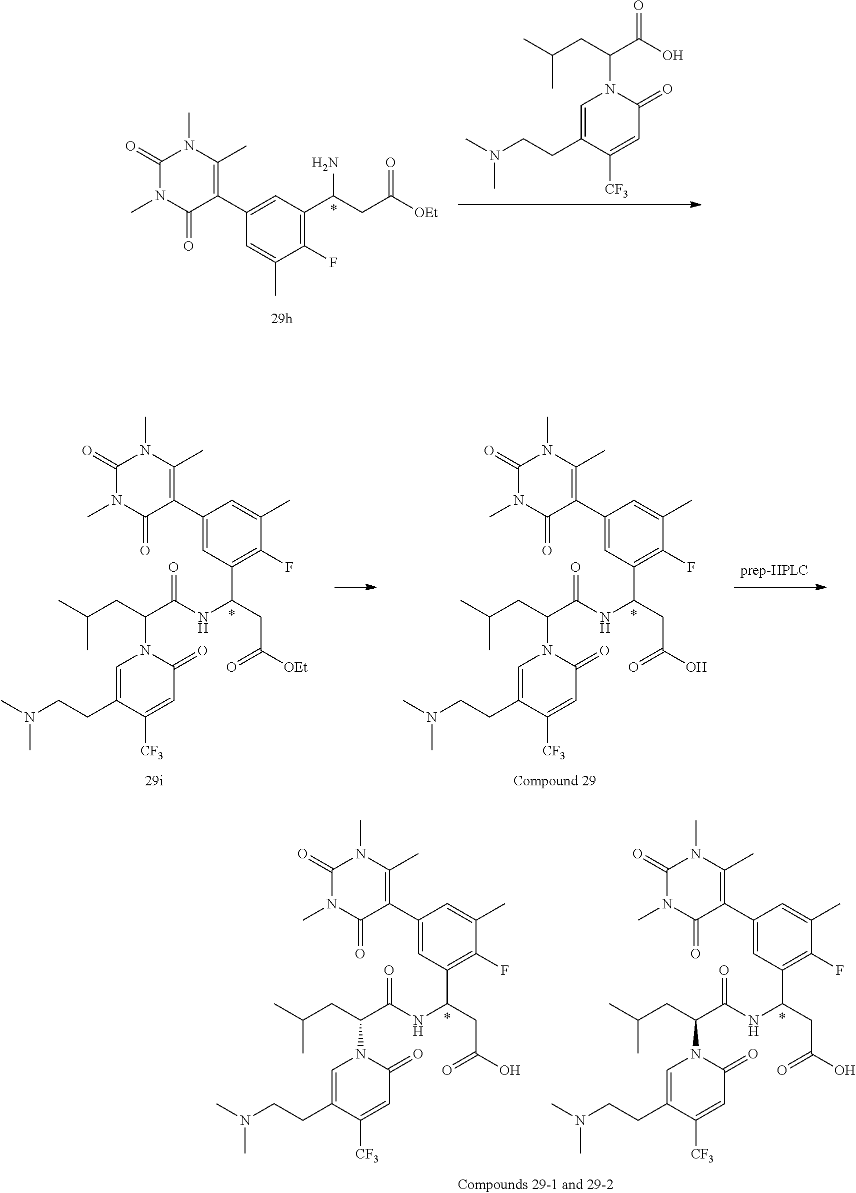

### Step 1: Synthesis of 29b

29a (10 g, 46.8 mmol) was dissolved in THF (120 mL), (R)-(+)-tert-butylsulfinamide (6.7 g, 55.3 mmol) and tetraethyl titanate (42.1 g, 184.3 mmol) were successively added, and the mixture was stirred at 50° C. under nitrogen protection for 3 h. The reaction mixture was cooled to room temperature, followed by the addition of ice water (100 mL). The mixture was filtered and the filter cake was washed with ethyl acetate (20 mL×2). The filtrate was allowed to stand for layering. The organic phase was collected and the aqueous phase was extracted with ethyl acetate (30 mL×3). The organic phases were combined, dried over anhydrous sodium sulfate, filtered and concentrated to afford crude 29b.

LC-Ms m/z (ESI): 307.1 [M+H]⁺.

### Step 2: Synthesis of 29c

Zinc powder (17.2 g, 262.4 mmol) was added to anhydrous THF (80 mL), followed by CuCl (5.6 g, 56.2 mmol). The mixture was subjected to nitrogen replacement three times and reacted at 60° C. for 1 h. At 0° C., a solution of ethyl bromoacetate (15.7 g, 93.7 mmol) in THF (10 mL) was slowly added dropwise and the resulting mixture was stirred at 60° C. for 1 h. At 0° C., a solution of 29b (6 g, 18.7 mmol) in THF (10 mL) was slowly added dropwise and the mixture was stirred at 0° C. for 3 h. The reaction mixture was filtered and the filtrate was quenched with 1 N HCl, and extracted with ethyl acetate (50 mL×3). The organic phases were combined, dried over anhydrous sodium sulfate, filtered and concentrated and the residue was purified by silica gel column to afford 29c (5.8 g, yield: 75.8%).

LC-Ms m/z (ESI): 408.4 [M+H]⁺.

### Step 3: Synthesis of 29d

29c (1 g, 2.45 mmol) was dissolved in THF (12 mL), a 4 M hydrogen chloride 1,4-dioxane solution (12 mL) was added, and the mixture was stirred at room temperature for 2 h. The reaction mixture was concentrated under reduced pressure to afford crude 29d.

LC-Ms m/z (ESI): 305.1[M+H]⁺.

### Step 4: Synthesis of 29e

29d (1 g, 3.29 mmol) was dissolved in THF (15 mL) and water (20 mL), sodium carbonate (1.1 mg, 9.8 mmol) was added, a solution of di-tert-butyl dicarbonate (860 mg, 3.9 mmol) in THF (5 mL) was added dropwise, and the mixture was stirred at room temperature for 3 h. The reaction mixture was extracted with ethyl acetate (20 mL×2), and the organic phases were combined, dried over anhydrous sodium sulfate, filtered and concentrated to afford a crude, which was subjected to silica gel column chromatography to afford 29e (1.2 g, yield: 90.2%).

LC-Ms m/z (ESI): 405.2[M+H]⁺.

### Step 5: Synthesis of 29f

29e (1 g, 2.47 mmol) was dissolved in ultra-dry 1,4-dioxane (20 mL), bis(pinacolato)diboron (750 mg, 3.0 mmol), Pd(dppf)Cl₂ (100 mg, 0.12 mmol) and potassium acetate (480 mg, 4.9 mmol) were successively added, and the resulting mixture was stirred at 110° C. under nitrogen protection for 3 h.

The reaction mixture was cooled to room temperature and filtered and the filtrate was concentrated under reduced pressure. The crude was dissolved in ethyl acetate (50 mL), and water (50 mL) was added for layering and extraction. The aqueous phase was extracted with ethyl acetate (50 mL×2). The organic phases were combined, dried over anhydrous sodium sulfate, filtered and concentrated under reduced pressure and the residue was subjected to silica gel column chromatography to afford 29f (1 g, yield: 89.7%).

LC-Ms m/z (ESI): 452.3[M+H]⁺.

### Step 6: Synthesis of 29g

29f (1 g, 2.2 mmol) was dissolved in 1,4-dioxane (20 mL) and water (2 mL), and 5-bromo-1,3,6-trimethylpyrimidine-2,4(1H, 3H)-dione (620 mg, 2.7 mmol), Pd(dppf)Cl₂ (163 mg, 0.2 mmol) and potassium carbonate (920 mg, 6.7 mmol) were successively added. Under nitrogen protection, the mixture was stirred at 100° C. for 5 h. The reaction mixture was cooled to room temperature and filtered and the filtrate was concentrated under reduced pressure. The crude was dissolved in ethyl acetate (50 mL), and water (50 mL) was added for layering and extraction. The organic phase was collected and the aqueous phase was extracted with ethyl acetate (50 mL×2). The organic phases were combined, dried over anhydrous sodium sulfate, filtered and spun to dryness to afford a crude, which was subjected to silica gel column chromatography to afford 29g (900 mg, yield: 84.9%).

LC-Ms m/z (ESI): 478.5[M+H]⁺.

### Step 7: Synthesis of 29h

29g (900 mg, 1.88 mmol) was dissolved in THF (12 mL), a 4 N hydrogen chloride 1,4-dioxane solution (12 mL) was added, and the mixture was stirred at room temperature for 2 h. The reaction mixture was concentrated under reduced pressure to afford crude 29h.

LC-Ms m/z (ESI): 378.4[M+H]⁺.

### Step 8: Synthesis of 29i

17h (640 mg, 1.85 mmol) was dissolved in DMF (10 mL), 29h (700 mg, 1.85 mmol), HATU (1.4 g, 3.7 mmol) and DIPEA (960 mg, 7.44 mmol) were successively added, and the mixture was reacted at room temperature for 1 h. Water (50 mL) was added and the resulting mixture was extracted with ethyl acetate (15 mL×3). The organic phases were combined and washed with saturated brine. The organic phase was collected, dried over anhydrous sodium sulfate, filtered and concentrated and the residue was subjected to silica gel column chromatography to afford 29i (520 mg, yield: 39.7%).

LC-Ms m/z (ESI): 708.7[M+H]⁺.

### Step 9: Synthesis of Compounds 29-1 and 29-2

29i (520 mg, 0.73 mmol) was dissolved in THF (5 mL) and water (1.5 mL), 1,5,7-triazabicyclo[4.4.0]dec-5-ene (200 mg, 1.44 mmol) was added, and the resulting mixture was reacted at room temperature for 6 h. The reaction mixture was adjusted with 1 N hydrochloric acid to pH 5-6, and concentrated under reduced pressure to afford crude compound 29, which was separated and purified by prep-HPLC (composition of mobile phases: mobile phase A: acetonitrile, mobile phase B: water (containing 0.1% ammonium acetate)) to afford compound 29-1 (80 mg, retention time=3.306 min, yield: 16.1%) and compound 29-2 (60 mg, retention time=3.234 min, yield: 12.1%).

Compound 29-1: LC-Ms m/z (ESI): 680.7[M+H]⁺.

¹H NMR (400 MHz, DMSO-d₆) δ 9.58-9.33 (m, 1H), 7.78 (s, 1H), 7.02-6.90 (m, 2H), 6.74 (s, 1H), 5.66-5.56 (m, 1H), 5.37-5.26 (m, 1H), 3.41 (s, 3H), 3.22 (s, 3H), 2.66-2.53 (m, 2H), 2.46-2.30 (m, 4H), 2.22 (s, 3H), 2.16 (s, 6H), 2.11 (s, 3H), 1.90-1.78 (m, 1H), 1.77-1.66 (m, 1H), 1.27-1.14 (m, 1H), 0.88-0.73 (s, 6H).

Compound 29-2 LC-Ms m/z (ESI): 680.7[M+H]⁺.

¹H NMR (400 MHz, DMSO-d₆) δ 9.36 (m, 1H), 7.76 (s, 1H), 6.97-6.87 (m, 2H), 6.68 (s, 1H), 5.61-5.53 (m, 1H), 5.36-5.27 (m, 1H), 3.40 (s, 3H), 3.21 (s, 3H), 2.61-2.51 (m, 2H), 2.49-2.47 (m, 2H), 2.41-2.28 (m, 2H), 2.20 (s, 3H), 2.15 (s, 6H), 2.06-1.91 (m, 4H), 1.83-1.74 (m, 1H), 1.35-1.21 (m, 1H), 0.91-0.78 (m, 6H).

### Example 30: Preparation of Compound 30 and Stereoisomer Thereof

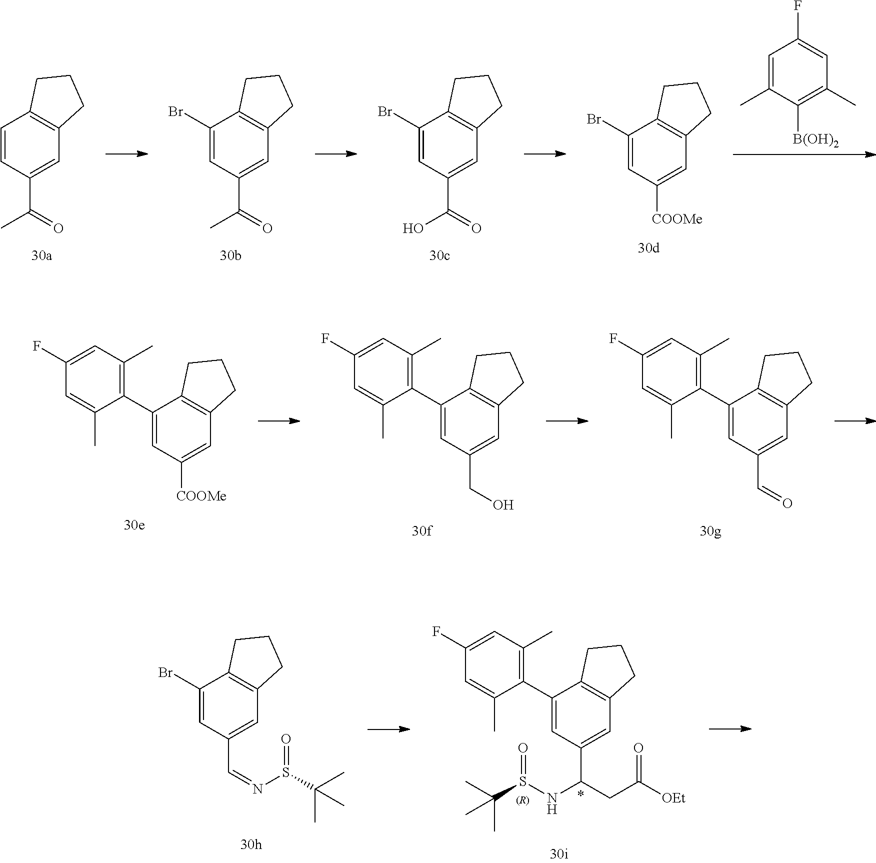

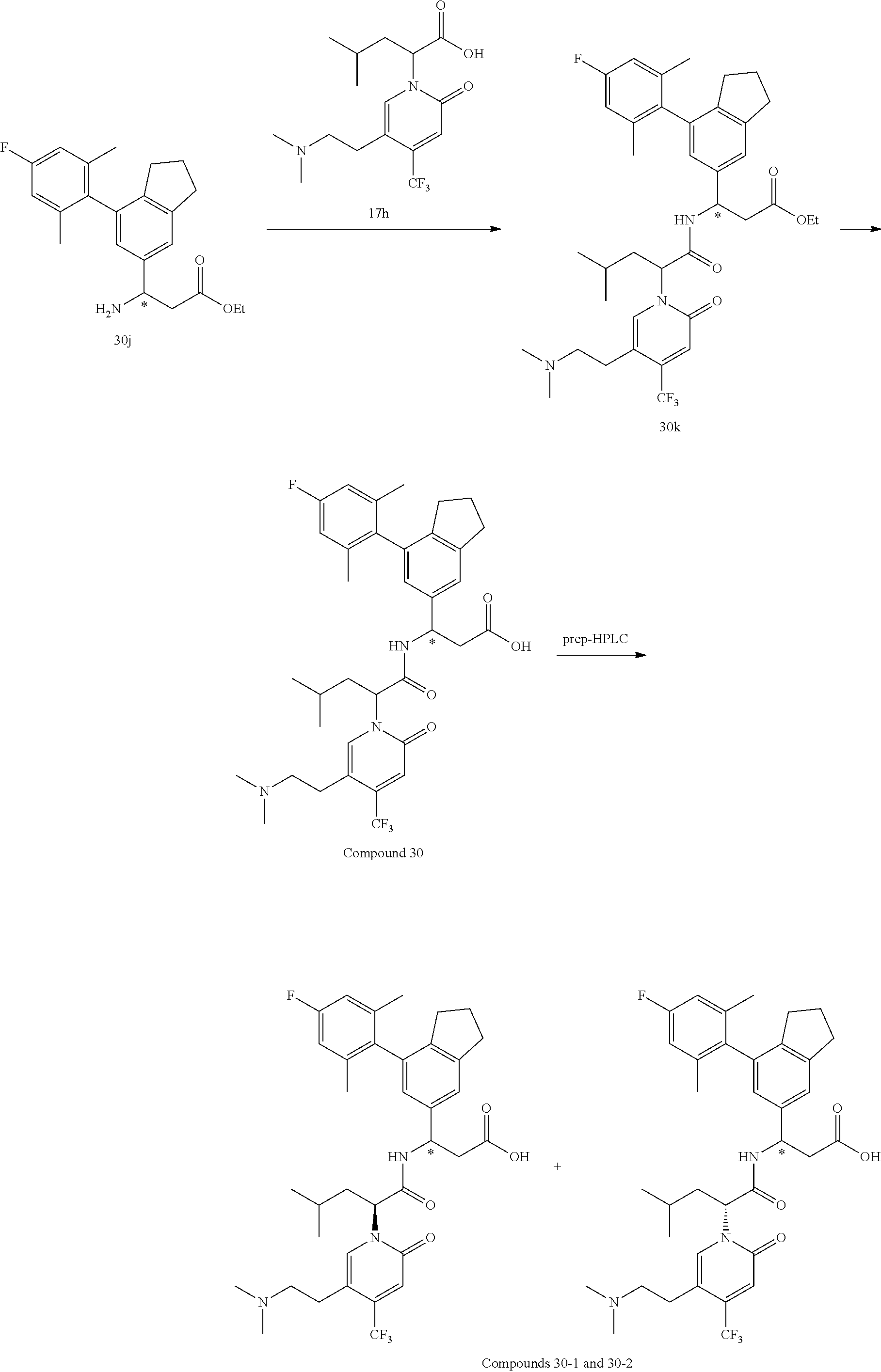 [SMILES: CCOC(=O)CC(N)c1cc2c(c(-c3c(C)cc(F)cc3C)c1)CCC2 ; CC(C)CC(C(=O)O)n1cc(CCN(C)C)c(C(F)(F)F)cc1=O ; CCOC(=O)CC(NC(=O)C(CC(C)C)n1cc(CCN(C)C)c(C(F)(F)F)cc1=O)c1cc2c(c(-c3c(C)cc(F)cc3C)c1)CCC2 ; Cc1cc(F)cc(C)c1-c1cc(C(CC(=O)O)NC(=O)C(CC(C)C)n2cc(CCN(C)C)c(C(F)(F)F)cc2=O)cc2c1CCC2 ; Cc1cc(F)cc(C)c1-c1cc(C(CC(=O)O)NC(=O)[C@H](CC(C)C)n2cc(CCN(C)C)c(C(F)(F)F)cc2=O)cc2c1CCC2 ; Cc1cc(F)cc(C)c1-c1cc(C(CC(=O)O)NC(=O)[C@@H](CC(C)C)n2cc(CCN(C)C)c(C(F)(F)F)cc2=O)cc2c1CCC2]

### Step 1: Synthesis of 30b

Under nitrogen protection, 30a (6.3 g, 39.34 mmol) was dissolved in dichloromethane (150 mL), and anhydrous aluminum chloride (13.11 g, 98.35 mmol) was added. The reaction mixture was reacted at room temperature for 5 min, a solution of bromine (8.80 g, 55.08 mmol) in dichloromethane (50 mL) was added, and the resulting mixture was reacted at room temperature for 2 h. The reaction liquid was poured into ice water (200 mL), dichloromethane (100 mL) was added, and the resulting mixture was washed with saturated brine (80 mL), dried over anhydrous sodium sulfate, filtered and concentrated. The residue was subjected to silica gel column chromatography to afford 30b (8.0 g, 85.05%).

LC-Ms m/z (ESI): 241.1 [M+H]⁺.

### Step 2: Synthesis of 30c

30b (8.1 g, 33.88 mmol) was dissolved in DME (100 mL), 1 M sodium hypochlorite solution (188 mL) and 10 N sodium hydroxide solution (18.5 mL) were added, and the mixture was reacted at 50° C. for 1 h. Water (150 mL) was added, the resulting mixture was extracted with diethyl ether (200 mL), and the aqueous phase was adjusted with hydrochloric acid to pH 1-2 and extracted with diethyl ether (150 ml×2). The organic phases were combined, dried over anhydrous sodium sulfate and concentrated to afford 30c (7.6 g, 93.05%).

LC-Ms m/z (ESI): 238.9 [M+H]⁺.

### Step 3: Synthesis of 30d

30c (7.6 g, 31.52 mmol) was dissolved in methanol (50 mL), thionyl chloride (11.25 g, 94.56 mmol) was added, and the mixture was reacted at 70° C. for 2 h. The reaction mixture was concentrated under reduced pressure and subjected to silica gel column chromatography to afford 30d (8.0 g, 99.49%).

LC-Ms m/z (ESI): 255.0 [M+H]⁺.

### Step 4: Synthesis of 30e

Under nitrogen protection, 30d (2 g, 7.84 mmol), 4-fluoro2,6-dimethylphenylboronic acid (2.63 g, 15.68 mmol) and cesium carbonate (7.66 g, 23.52 mmol) were added to 1,4-dioxane (30.0 mL) and water (3.0 mL), followed by Pd(PPh₃)₄ (1.81 g, 1.57 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 30e (2.2 g, yield: 94.05%).

### Step 5: Synthesis of 30f

Under nitrogen protection, 30e (2.13 g, 7.14 mmol) was dissolved in dry THF (30 mL) and at 0° C., lithium aluminum hydride (406 mg, 10.71 mmol) was slowly added. The resulting mixture was reacted at room temperature for 1 h. 10% sodium sulfate aqueous solution (20 mL) was added and the mixture was filtered. The solvent was removed under reduced pressure to afford crude 30f.

### Step 6: Synthesis of Compound 30g

Under nitrogen protection, crude 30f (1.87 g, 6.92 mmol) was dissolved in dry dichloromethane (35 mL), sodium bicarbonate (1.16 g, 13.84 mmol) and Dess-Martin periodinane (3.81 g, 9.00 mmol) were added, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate and filtered. The solvent was removed under reduced pressure and the residue was subjected to column chromatography on a silica gel column to afford 30g (1.63 g, 87.68%).

### Step 7: Synthesis of 30h

Under nitrogen protection, 30g (1.61 g, 6.00 mmol) and R-tert-butylsulfinamide (1.01 g, 9.00 mmol) were dissolved in THF (35 mL), tetraethyl titanate (2.05 g, 9.00 mmol) was slowly added, and the resulting mixture was reacted at 45° C. for 15 h and concentrated under reduced pressure to afford a crude, which was subjected to flash column chromatography on a silica gel column to afford 30h (1.73 g, yield: 77.61%).

LC-Ms m/z (ESI): 372.1 [M+H]⁺.

### Step 8: Synthesis of 30i

Zinc powder (915 mg, 14 mmol) was added to dry THF (5 mL), the mixture was subjected to nitrogen replacement three times, CuCl (297 mg, 3 mmol) was added, and the resulting mixture was reacted at 60° C. for 2 h. The reaction mixture was cooled to room temperature, ethyl bromoacetate (835 mg, 5 mmol) was slowly added, and the resulting mixture was reacted at 60° C. for 1 h and cooled to 0° C. A solution of 30h (371 mg, 1 mmol) in THF (1 mL) was added and the mixture was stirred at 0° C. for 3 h. The reaction mixture was filtered through celite and saturated ammonium chloride solution (20 mL) was added. The resulting mixture was extracted with ethyl acetate (30 ml×3), washed with saturated brine (20 mL), dried over anhydrous sodium sulfate, filtered and concentrated under reduced pressure. The residue was subjected to flash column chromatography to afford 30i (386 mg, yield: 83.98%).

LC-Ms m/z (ESI): 460.2 [M+H]⁺.

### Step 9: Synthesis of 30j

30i (272 mg, 0.27 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude 30j.

LC-Ms m/z (ESI): 356.3 [M+H]⁺.

### Step 10: Synthesis of Compound 30k

Under nitrogen protection, 17h (101 mg, 0.29 mmol) was dissolved in dry DMF (1.5 mL), and HATU (220 mg, 0.58 mmol) and DIPEA (225 mg, 1.74 mmol) were added. At room temperature, the reaction mixture was stirred for 40 min, and then crude 30j (103 mg, 0.29 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 30k (145 mg, yield: 72.91%).

LC-Ms m/z (ESI): 687.0 [M+H]⁺.

### Step 11: Synthesis of Compounds 30-1 and 30-2

30k (248 mg, 0.36 mmol) was dissolved in THF (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 30, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 30-1 (31 mg, retention time=5.624 min, yield: 12.76%) and compound 30-2 (42 mg, retention time=5.701 min, yield: 17.74%).

Compound 30-1:

LC-Ms m/z (ESI): 658.3 [M+H]⁺.

¹H NMR (400 MHz, DMSO-d₆) δ 8.92 (d, 1H), 7.79 (s, 1H), 7.12 (s, 1H), 6.96-6.87 (m, 2H), 6.73 (d, 1H), 6.67 (s, 1H), 5.61-5.52 (m, 1H), 5.20-5.11 (m, 1H), 2.92-2.85 (m, 2H), 2.72-2.66 (m, 2H), 2.63-2.53 (m, 2H), 2.40-2.28 (m, 4H), 2.16 (s, 6H), 1.99-1.89 (m, 3H), 1.85 (s, 3H), 1.80 (s, 3H), 1.79-1.71 (m, 1H), 1.39-1.27 (m, 1H), 0.88 (d, 3H), 0.83 (d, 3H).

Compound 30-2:

LC-Ms m/z (ESI): 658.3 [M+H]⁺.

¹H NMR (400 MHz, DMSO-d₆) δ 8.98 (d, 1H), 7.81 (s, 1H), 7.15 (s, 1H), 6.98 (s, 1H), 6.95 (s, 1H), 6.78 (d, 1H), 6.74 (s, 1H), 5.62-5.55 (m, 1H), 5.18-5.09 (m, 1H), 2.96-2.87 (m, 2H), 2.72-2.53 (m, 4H), 2.44-2.31 (m, 4H), 2.17 (s, 6H), 2.02-1.92 (m, 2H), 1.90 (s, 3H), 1.89 (s, 3H), 1.83-1.73 (m, 1H), 1.67-1.56 (m, 1H), 1.26-1.14 (m, 1H), 0.82-0.73 (m, 6H).

### Example 31: Preparation of Compound 31 and Stereoisomer Thereof

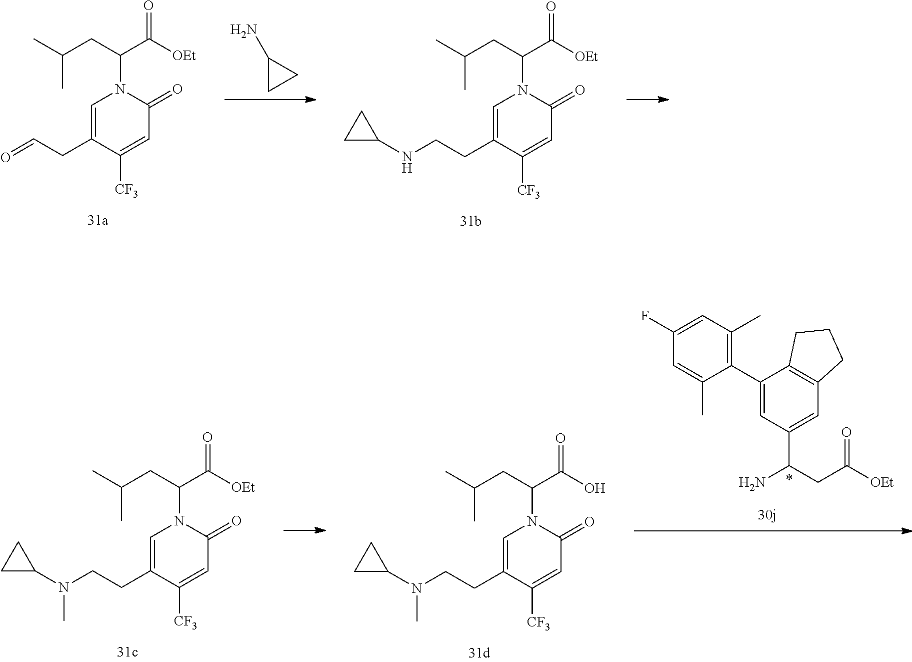

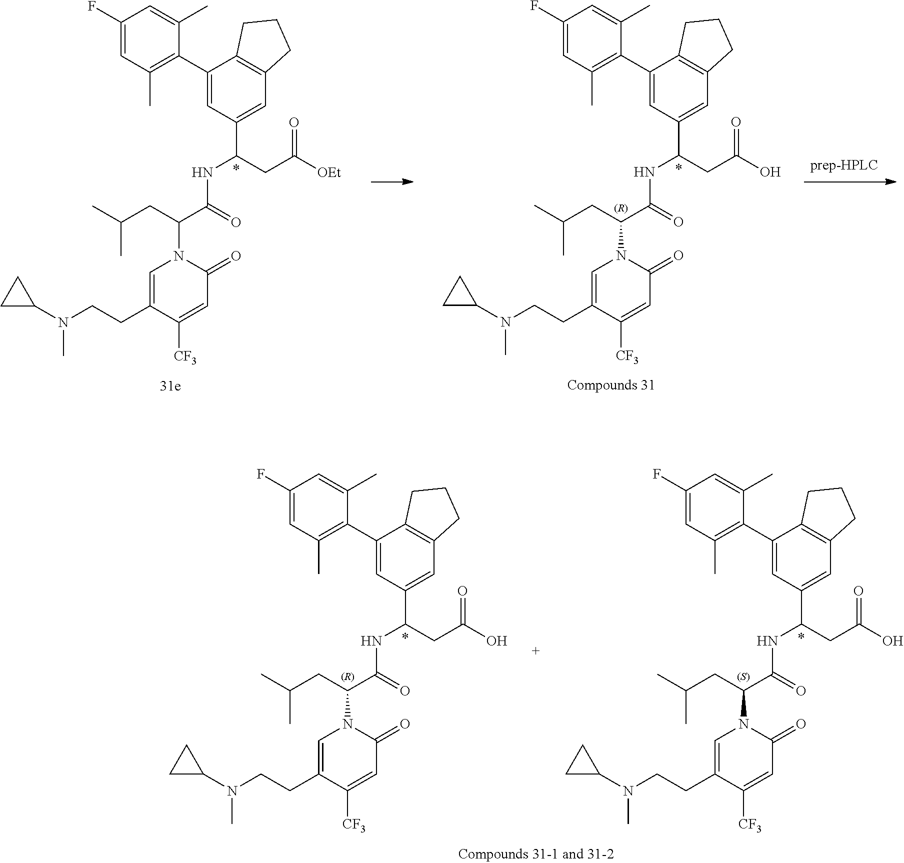

### Step 1: Synthesis of 31b

Under nitrogen protection, 31a (see WO 2021076890 A1 for the synthetic method) (680 mg, 1.96 mmol), cyclopropylamine (224 mg, 3.92 mmol), and acetic acid (59 mg, 0.98 mmol) were dissolved in DCE (8 mL), the mixture was reacted at room temperature for 1 h, and sodium triacetoxyborohydride (831 mg, 3.92 mmol) was added. The resulting mixture was reacted at room temperature overnight and filtered through celite. Dichloromethane (30 mL) was added and the mixture was washed with saturated sodium bicarbonate solution (10 mL), dried over anhydrous sodium sulfate and filtered. The filtrate was concentrated under reduced pressure and the residue was subjected to column chromatography on a silica gel column to afford 31b (370 mg, yield: 48.60%).

LC-Ms m/z (ESI): 389.4 [M+H]⁺.

### Step 2: Synthesis of 31c

Under nitrogen protection, 31b (311 mg, 0.80 mmol), paraformaldehyde (144 mg, 1.6 mmol) and acetic acid (24 mg, 0.40 mmol) were dissolved in DCE (4 mL), the mixture was reacted at room temperature for 1 h, and sodium triacetoxyborohydride (339 mg, 1.60 mmol) was added. The resulting mixture was reacted at room temperature overnight. The reaction mixture was filtered through celite, dichloromethane (30 mL) was added, and the resulting mixture was washed with saturated sodium bicarbonate solution (10 mL), dried over anhydrous sodium sulfate and filtered. The filtrate was concentrated under reduced pressure and then subjected to column chromatography on a silica gel column to afford 31c (260 mg, yield: 80.76%).

LC-Ms m/z (ESI): 403.2 [M+H]⁺.

### Step 3: Synthesis of 31d

31c (295 mg, 0.73 mmol) was dissolved in THF (3.6 mL) and water (1.2 mL), lithium hydroxide monohydrate (92 mg, 2.19 mmol) was added, and the resulting mixture was reacted at room temperature for 5 h. The reaction mixture was adjusted with 1 N hydrochloric acid to pH 5-6 and concentrated under reduced pressure. The residue was subjected to C18 reverse phase column chromatography to afford 31d (260 mg, 95.13%).

LC-Ms m/z (ESI): 375.1 [M+H]⁺.

### Step 4: Synthesis of 31e

Under nitrogen protection, 31d (153 mg, 0.41 mmol) was dissolved in dry DMF (2.5 mL), HATU (312 mg, 0.82 mmol) and DIPEA (318 mg, 2.46 mmol) were added, and the resulting mixture was stirred at room temperature for 40 min. Crude 30j (144 mg, 0.41 mmol) was added and the mixture was reacted at room temperature overnight. Ethyl acetate (80 mL) was added and the resulting reaction mixture was successively washed with water (20 mL×2) and saturated brine (20 mL×1), dried over anhydrous sodium sulfate and filtered. The filtrate was concentrated under reduced pressure and subjected to column chromatography on a silica gel column to afford 31e (160 mg, yield: 54.82%).

LC-Ms m/z (ESI): 712.3 [M+H]⁺.

### Step 5: Synthesis of Compounds 31-1 and 31-2

31e (160 mg, 0.22 mmol) was dissolved in THF (3 mL) and water (1 mL), lithium hydroxide monohydrate (28 mg, 0.66 mmol) was added, and the resulting mixture was reacted at room temperature for 5 h. The reaction mixture was adjusted with 1 N hydrochloric acid to pH 5-6 and concentrated under reduced pressure to afford crude compound 31, which was separated and purified by prep-HPLC (instrument: waters 2767 preparative chromatographic column: SunFire @Prep C18 (19 mm×150 mm); composition of mobile phases: mobile phase A: acetonitrile, and mobile phase B: water (containing 0.1% ammonium acetate)) to afford compound 31-1 (19 mg, retention time=5.684 min, yield: 12.63%) and compound 31-2 (21 mg, retention time=5.749 min, yield: 13.96%).

LC-Ms m/z (ESI): 684.3 [M+H]⁺.

Compound 31-1:

LC-Ms m/z (ESI): 684.3 [M+H]⁺.

¹H NMR (400 MHz, DMSO-d₆) δ 8.91 (d, 1H), 7.76 (s, 1H), 7.12 (s, 1H), 7.00-6.86 (m, 2H), 6.73 (d, 1H), 6.67 (s, 1H), 5.62-5.53 (m, 1H), 5.20-5.10 (m, 1H), 2.92-2.84 (m, 2H), 2.74-2.55 (m, 6H), 2.33 (t, 2H), 2.27 (s, 3H), 1.99-1.89 (m, 2H), 1.89-1.81 (m, 4H), 1.79 (s, 3H), 1.77-1.71 (m, 1H), 1.67-1.61 (m, 1H), 1.38-1.23 (m, 1H), 0.88 (d, 3H), 0.84 (d, 3H), 0.42-0.37 (m, 2H), 0.30-0.23 (m, 1H), 0.21-0.14 (m, 1H).

Compound 31-2

LC-Ms m/z (ESI): 684.3 [M+H]⁺:

¹H NMR (400 MHz, DMSO-d₆) δ 9.00 (d, 1H), 7.81 (s, 1H), 7.15 (s, 1H), 6.98 (s, 1H), 6.96 (s, 1H), 6.78 (d, 1H), 6.74 (s, 1H), 5.64-5.56 (m, 1H), 5.18-5.09 (m, 1H), 2.91 (t, 2H), 2.72-2.54 (m, 6H), 2.37 (t, 2H), 2.27 (s, 3H), 2.01-1.91 (m, 2H), 1.91-1.88 (m, 6H), 1.77-1.51 (m, 3H), 1.23-1.09 (m, 1H), 0.81-0.73 (m, 6H), 0.41-0.36 (m, 2H), 0.27-0.23 (m, 1H), 0.20-0.12 (m, 1H).

### Example 32: Preparation of Compound 32 and Stereoisomer Thereof

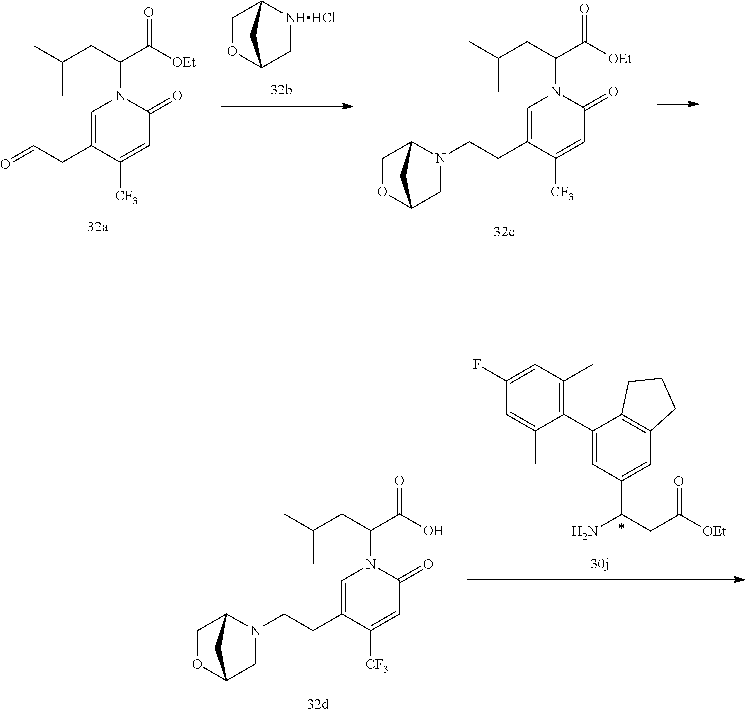

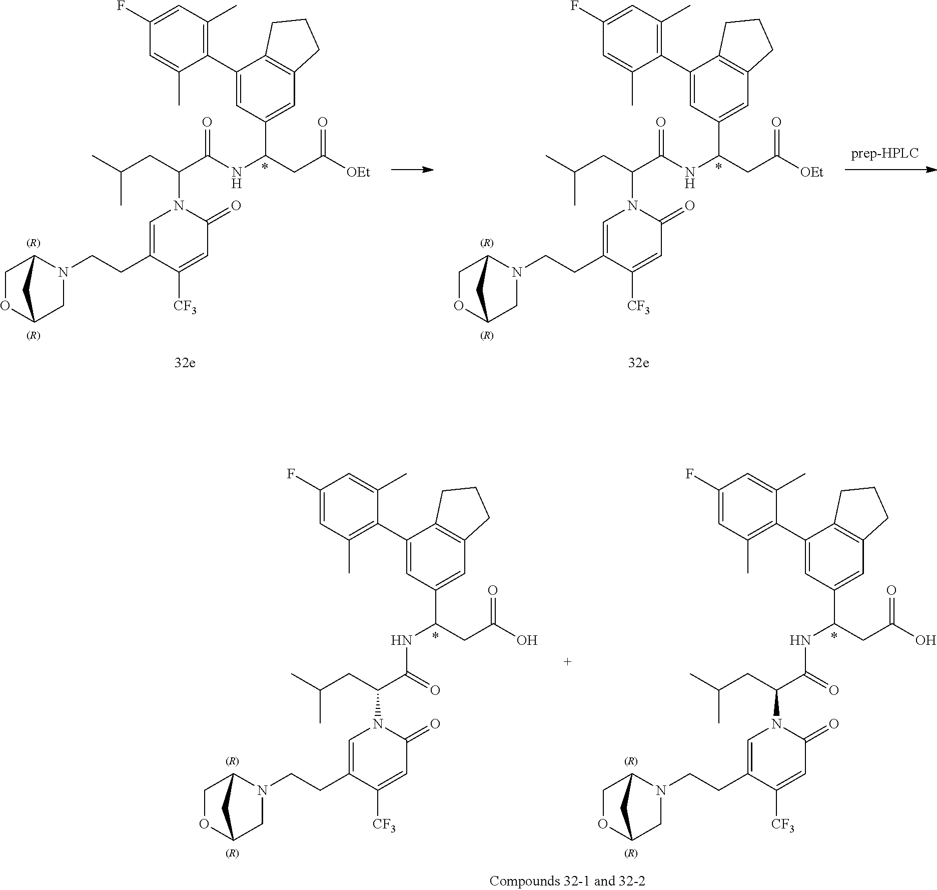

### Step 1: Synthesis of 32c

Under nitrogen protection, 32a (1.18 g, 3.4 mmol) and 32b (0.46 g, 3.4 mmol) were dissolved in 1,2-dichloroethane (17 mL) and acetic acid (103 mg, 1.7 mmol) was then added. After the resulting mixture was reacted at room temperature for 1 h, sodium triacetylborohydride (1.44 g, 6.8 mmol) was added. The reaction mixture was reacted at room temperature overnight, filtered and concentrated and the residue was subjected to silica gel column chromatography to afford 32c (860 mg, 58.76%).

LC-Ms m/z (ESI): 431.2 [M+H]⁺.

### Step 2: Synthesis of 32d

32c (860 mg, 2.00 mmol) was dissolved in THF (12 mL) and water (4 mL), lithium hydroxide monohydrate (252 mg, 6.00 mmol) was added, and the resulting mixture was reacted at room temperature for 5 h. The reaction mixture was adjusted with 1 N hydrochloric acid to pH 5-6 and concentrated under reduced pressure to afford a crude, which was purified by C18 column to afford 32d (630 mg, 78.28%).

LC-Ms m/z (ESI): 403.1 [M+H]⁺.

### Step 3: Synthesis of Compound 32e

Under nitrogen protection, 32d (128 mg, 0.36 mmol) was dissolved in dry DMF (3.0 mL), and HATU (274 mg, 0.72 mmol) and DIPEA (279 mg, 2.16 mmol) were added. At room temperature, the reaction mixture was stirred for 40 min, and then crude 30j (140 mg, 0.36 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 32e (425 mg, crude).

LC-Ms m/z (ESI): 741.0 [M+H]⁺.

### Step 4: Synthesis of Compounds 32-1 and 32-2

Crude 32e (425 mg) was dissolved in THF (4 mL) and water (1 mL), lithium hydroxide monohydrate (45 mg, 1.08 mmol) was added, and the resulting mixture was reacted at room temperature for 5 h.

The reaction mixture was adjusted with 1 N hydrochloric acid to pH 5-6 and concentrated under reduced pressure to afford crude compound 32, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 32-1 (30 mg, retention time=4.235 min, three-step yield: 11.71%) and compound 32-2 (30 mg, retention time=4.341 min, three-step yield: 11.71%).

Compound 32-1:

LC-Ms m/z (ESI): 713.1 [M+H]⁺.

¹H NMR (400 MHz, DMSO-d₆) δ 8.95 (d, 1H), 7.84 (s, 1H), 7.12 (s, 1H), 6.96-6.87 (m, 2H), 6.73 (d, 1H), 6.67 (s, 1H), 5.61-5.54 (m, 1H), 5.21-5.12 (m, 1H), 4.32-4.30 (m, 1H), 3.77 (d, 1H), 3.49-3.41 (m, 3H), 2.91-2.85 (m, 2H), 2.79-2.73 (m, 1H), 2.71-2.54 (m, 5H), 2.43-2.30 (m, 3H), 1.97-1.87 (m, 2H), 1.84 (s, 3H), 1.83-1.74 (m, 4H), 1.71-1.65 (m, 1H), 1.56-1.50 (m, 1H), 1.39-1.29 (m, 1H), 0.88 (d, 3H), 0.84 (d, 3H).

Compound 32-2:

LC-Ms m/z (ESI): 713.1 [M+H]⁺.

¹H NMR (400 MHz, DMSO-d₆) δ 9.02 (d, 1H), 7.87 (s, 1H), 7.15 (d, 1H), 6.98 (s, 1H), 6.96 (s, 1H), 6.78 (d, 1H), 6.73 (s, 1H), 5.64-5.56 (m, 1H), 5.17-5.09 (m, 1H), 4.33-4.29 (m, 1H), 3.80 (d, 1H), 3.48-3.45 (m, 2H), 2.94-2.87 (m, 2H), 2.82-2.77 (m, 1H), 2.73-2.64 (m, 3H), 2.64-2.53 (m, 3H), 2.42-2.32 (m, 3H), 2.01-1.92 (m, 2H), 1.90 (s, 3H), 1.89 (s, 3H), 1.82-1.71 (m, 1H), 1.69-1.53 (m, 3H), 1.23-1.16 (m, 1H), 0.81-0.72 (m, 6H).

### Example 33: Preparation of Compound 33 and Stereoisomer Thereof

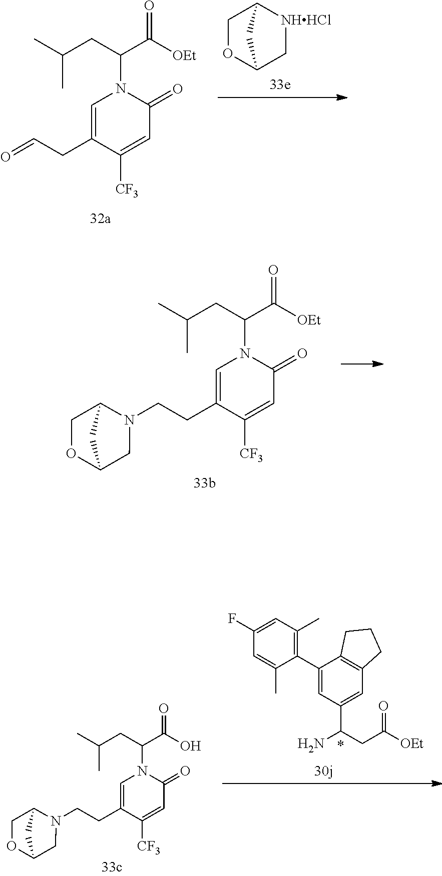

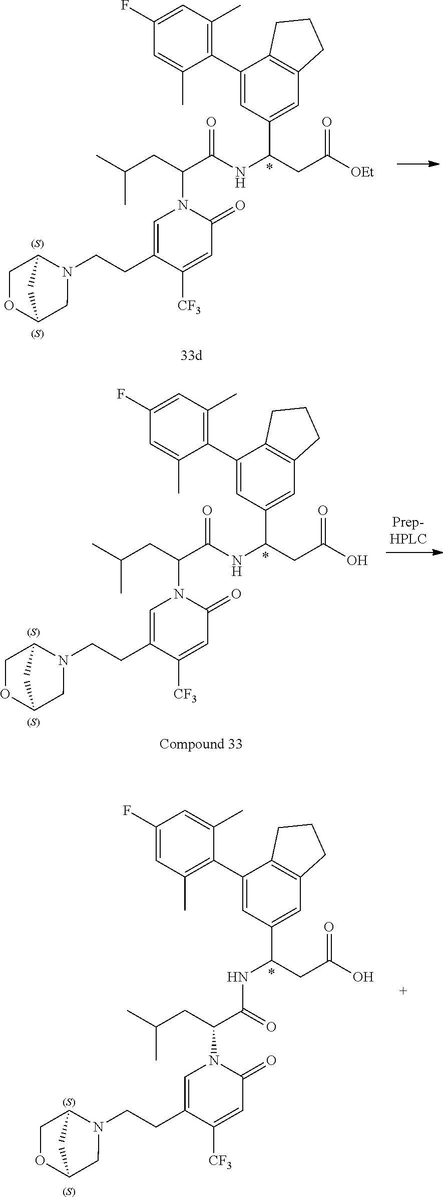

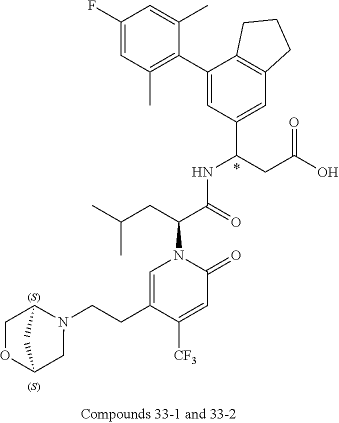

### Step 1: Synthesis of 33b

Under nitrogen protection, 32a (1.18 g, 3.4 mmol) and 33a (0.46 g, 3.4 mmol) were dissolved in 1,2-dichloroethane (17 mL) and acetic acid (103 mg, 1.7 mmol) was then added. After the resulting mixture was reacted at room temperature for 1 h, sodium triacetylborohydride (1.44 g, 6.8 mmol) was added. The reaction mixture was reacted at room temperature overnight, filtered and concentrated and the residue was subjected to silica gel column chromatography to afford 33b (790 mg, 53.98%).

LC-Ms m/z (ESI): 431.2 [M+H]⁺.

### Step 2: Synthesis of 33c

33b (780 mg, 1.81 mmol) was dissolved in THF (12 mL) and water (4 mL), lithium hydroxide monohydrate (228 mg, 5.43 mmol) was added, and the resulting mixture was reacted at room temperature for 5 h. The reaction mixture was adjusted with 1 N hydrochloric acid to pH 5-6 and concentrated under reduced pressure to afford a crude, which was purified by C18 column to afford 33c (630 mg, 86.50%).

LC-Ms m/z (ESI): 403.1 [M+H]⁺.

### Step 3: Synthesis of Compound 33d

Under nitrogen protection, 33c (128 mg, 0.36 mmol) was dissolved in dry DMF (3.0 mL), and HATU (274 mg, 0.72 mmol) and DIPEA (279 mg, 2.16 mmol) were added. At room temperature, the reaction mixture was stirred for 40 min, and then crude 30j (140 mg, 0.36 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 33d (450 mg, crude).

LC-Ms m/z (ESI): 740.3 [M+H]⁺.

### Step 4: Synthesis of Compounds 33-1 and 33-2

crude 33d (450 mg) was dissolved in THF (4 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 33, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 33-1 (25 mg, retention time=4.231 min, three-step yield: 8.78%) and compound 33-2 (25 mg, retention time=4.345 min, three-step yield: 8.78%).

Compound 33-1:

LC-Ms m/z (ESI): 713.1 [M+H]⁺.

¹H NMR (400 MHz, DMSO-d₆) δ 8.94 (d, 1H), 7.84 (s, 1H), 7.11 (s, 1H), 6.97-6.87 (m, 2H), 6.73 (d, 1H), 6.67 (s, 1H), 5.62-5.54 (m, 1H), 5.20-5.12 (m, 1H), 4.30 (t, 1H), 3.80 (d, 1H), 3.50-3.43 (m, 3H), 2.91-2.83 (m, 2H), 2.81-2.74 (m, 1H), 2.72-2.63 (m, 3H), 2.60-2.51 (m, 2H), 2.47-2.41 (m, 1H), 2.40-2.30 (m, 3H), 1.99-1.87 (m, 2H), 1.85 (s, 3H), 1.83-7.73 (m, 4H), 1.69-1.64 (m, 1H), 1.58-1.51 (m, 1H), 1.39-1.28 (m, 1H), 0.88 (d, 3H), 0.84 (d, 3H).

Compound 33-2:

LC-Ms m/z (ESI): 713.1 [M+H]⁺.

¹H NMR (400 MHz, DMSO-d₆) δ 9.05 (d, 1H), 7.87 (s, 1H), 7.15 (s, 1H), 6.98 (s, 1H), 6.95 (s, 1H), 6.78 (d, 1H), 6.73 (s, 1H), 5.64-5.55 (m, 1H), 5.17-5.06 (m, 1H), 4.31 (t, 1H), 3.78 (d, 1H), 3.50-3.44 (m, 3H), 2.95-2.86 (m, 2H), 2.80-2.74 (m, 1H), 2.71-2.52 (m, 5H), 2.44-2.31 (m, 3H), 2.03-1.92 (m, 2H), 1.91-1.88 (m, 6H), 1.81-1.73 (m, 1H), 1.72-1.58 (m, 2H), 1.58-1.51 (m, 1H), 1.22-1.13 (m, 1H), 0.81-0.73 (m, 6H).

### Example 34: Preparation of Compound 34 and Stereoisomer Thereof

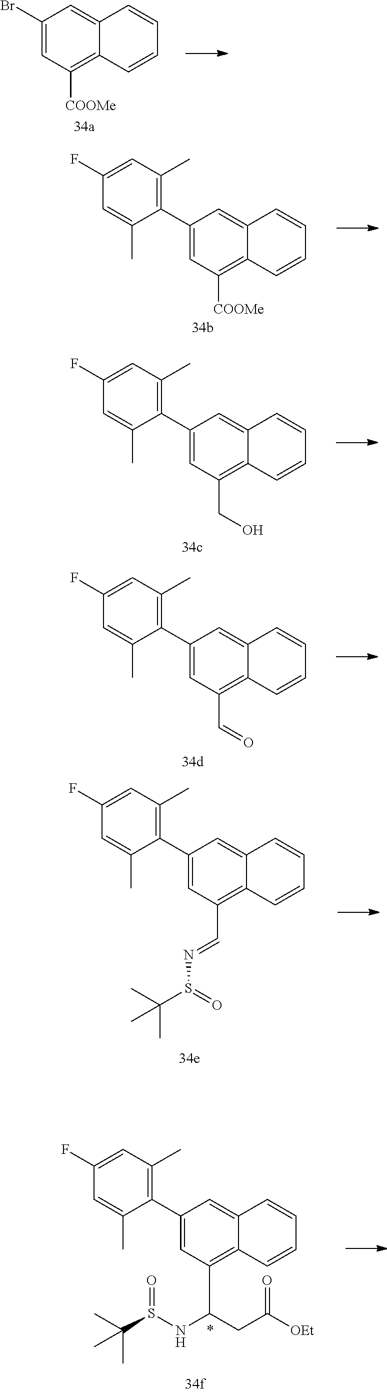 [SMILES: COC(=O)c1cc(Br)cc2ccccc12 ; COC(=O)c1cc(-c2c(C)cc(F)cc2C)cc2ccccc12 ; Cc1cc(F)cc(C)c1-c1cc(CO)c2ccccc2c1 ; Cc1cc(F)cc(C)c1-c1cc(C=O)c2ccccc2c1 ; Cc1cc(F)cc(C)c1-c1cc(/C=N/[S@](=O)C(C)(C)C)c2ccccc2c1 ; CCOC(=O)CC(N[S@](=O)C(C)(C)C)c1cc(-c2c(C)cc(F)cc2C)cc2ccccc12]

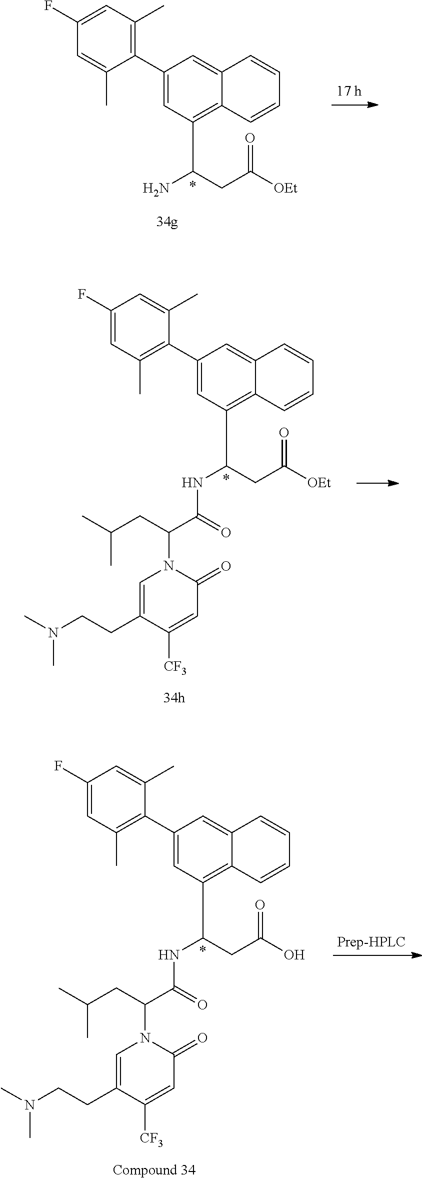 [SMILES: CCOC(=O)CC(N)c1cc(-c2c(C)cc(F)cc2C)cc2ccccc12 ; CCOC(=O)CC(NC(=O)C(CC(C)C)n1cc(CCN(C)C)c(C(F)(F)F)cc1=O)c1cc(-c2c(C)cc(F)cc2C)cc2ccccc12 ; Cc1cc(F)cc(C)c1-c1cc(C(CC(=O)O)NC(=O)C(CC(C)C)n2cc(CCN(C)C)c(C(F)(F)F)cc2=O)c2ccccc2c1]

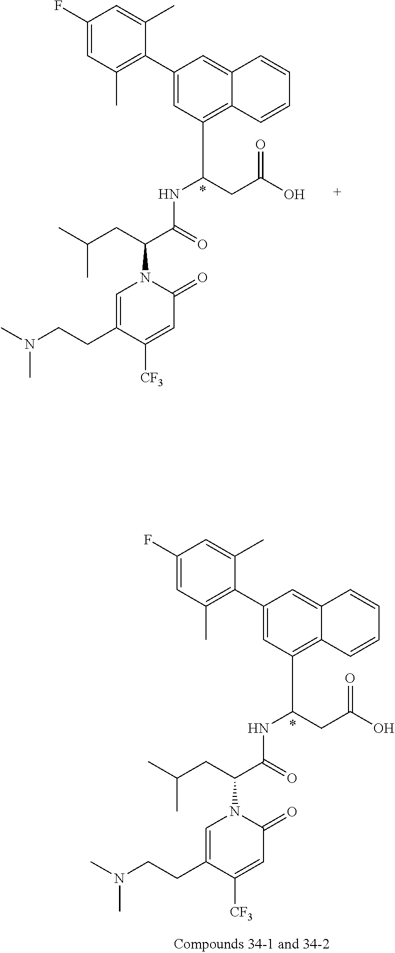 [SMILES: Cc1cc(F)cc(C)c1-c1cc(C(CC(=O)O)NC(=O)[C@H](CC(C)C)n2cc(CCN(C)C)c(C(F)(F)F)cc2=O)c2ccccc2c1 ; Cc1cc(F)cc(C)c1-c1cc(C(CC(=O)O)NC(=O)[C@@H](CC(C)C)n2cc(CCN(C)C)c(C(F)(F)F)cc2=O)c2ccccc2c1]

### Step 1: Synthesis of 34b

Under nitrogen protection, 34a (1.2 g, 4.53 mmol), 4-fluoro-2,6-dimethylphenylboronic acid (1.14 g, 6.79 mmol) and cesium carbonate (4.43 g, 13.59 mmol) were added to 1,4-dioxane (30.0 mL) and water (3.0 mL), followed by Pd(PPh₃)₄ (1.05 g, 0.91 mmol), and the mixture was reacted at 100° C. in a sealed tube for 3 h. The reaction system was cooled to room temperature and filtered through celite. Ethyl acetate (100 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 34b (1.3 g, yield: 93.07%).

### Step 2: Synthesis of 34c

Under nitrogen protection, 34b (1.25 g, 4.05 mmol) was dissolved in dry THF (25 mL) and at 0° C., lithium aluminum hydride (230 mg, 6.07 mmol) was slowly added. The resulting mixture was reacted at room temperature for 1 h. 10% sodium sulfate aqueous solution (20 mL) was added and the mixture was stirred for 20 min and filtered. The solvent was removed under reduced pressure to afford crude 34c.

### Step 3: Synthesis of 34d

Under nitrogen protection, crude 34c (1.13 g, 4.05 mmol) was dissolved in dry dichloromethane (20 mL), sodium bicarbonate (0.68 g, 8.1 mmol) and Dess-Martin periodinane (2.23 g, 5.26 mmol) were added, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate, filtered and concentrated under reduced pressure. The residue was subjected to silica gel column chromatography to afford 34d (909 mg, 80.64%).

### Step 4: Synthesis of 34e

Under nitrogen protection, 34d (909 mg, 3.22 mmol) and R-tert-butylsulfinamide (585.4 mg, 4.83 mmol) were dissolved in THF (22 mL), tetraethyl titanate (1.10 g, 4.83 mmol) was slowly added, and the resulting mixture was reacted at 45° C. for 15 h and concentrated under reduced pressure to afford a crude, which was subjected to flash column chromatography on a silica gel column to afford 34e (1.03 g, two-step total yield: 83.84%).

LC-Ms m/z (ESI): 382.6 [M+H]⁺.

### Step 5: Synthesis of 34f

Zinc powder (960 mg, 14 mmol) was added to dry THF (5 mL), the mixture was subjected to nitrogen replacement three times, CuCl (312 mg, 3.15 mmol) was added, and the resulting mixture was reacted at 60° C. for 2 h. The reaction mixture was cooled to room temperature, ethyl bromoacetate (876 mg, 5.25 mmol) was slowly added, and the resulting mixture was reacted at 60° C. for 1 h and cooled to 0° C. A solution of 34e (400 mg, 1 mmol) in THF (1 mL) was added and the mixture was stirred at 0° C. for 5 h. The reaction mixture was filtered through celite and saturated ammonium chloride solution (20 mL) was added. The resulting mixture was extracted with ethyl acetate (30 ml×3), washed with saturated brine (20 mL), dried over anhydrous sodium sulfate, filtered and concentrated under reduced pressure. The residue was subjected to flash column chromatography to afford 34f (480 mg, yield: 97.35%).

LC-Ms m/z (ESI): 470.3 [M+H]⁺.

### Step 6: Synthesis of 34g

34f (240 mg, 0.51 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude 34g (180 mg).

LC-Ms m/z (ESI): 366.3 [M+H]⁺.

### Step 7: Synthesis of Compound 34h

Under nitrogen protection, the above-mentioned crude 34g (180 mg) and 17h (143 mg, 0.41 mmol) were dissolved in dry DMF (3.5 mL), and HATU (310 mg, 0.82 mmol) and DIPEA (320 mg, 2.46 mmol) were added. The reaction mixture was reacted at room temperature overnight. Ethyl acetate (80 mL) was added and the resulting mixture was successively washed with water (20 mL×2) and saturated brine (20 mL×1), dried over anhydrous sodium sulfate and filtered. The filtrate was concentrated under reduced pressure and the residue was subjected to column chromatography on a silica gel column to afford 34h (360 mg).

LC-Ms m/z (ESI): 696.6 [M+H]⁺.

### Step 8: Synthesis of Compounds 34-1 and 34-2

34h (360 mg) was dissolved in THF (3 mL) and water (1 mL), lithium hydroxide monohydrate (56.65 mg, 1.35 mmol) was added, and the resulting mixture was reacted at room temperature for 5 h. The reaction mixture was adjusted with 1 N hydrochloric acid to pH 5-6 and concentrated under reduced pressure to afford crude compound 34, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 34-1 (30 mg, retention time=4.161 min, yield: 12.76%) and compound 34-2 (40 mg, retention time=4.242 min, yield: 17.74%).

Compound 34-1

LC-Ms m/z (ESI): 668.3 [M+H]⁺.

¹H NMR (400 MHz, Methanol-d₄) δ 8.24 (d, 1H), 7.93-7.85 (m, 2H), 7.60-7.49 (m, 3H), 7.25 (d, 1H), 6.92-6.86 (m, 1H), 6.85-6.81 (m, 1H), 6.73 (s, 1H), 6.20-6.14 (m, 1H), 5.76-5.69 (m, 1H), 3.13-3.03 (m, 2H), 2.99-2.82 (m, 3H), 2.81-2.72 (m, 7H), 2.08-2.00 (m, 5H), 1.84 (s, 3H), 1.50-1.42 (m, 1H), 0.98 (d, 3H), 0.95 (d, 3H).

Compound 34-2

LC-Ms m/z (ESI): 668.3 [M+H]⁺.

¹H NMR (400 MHz, Methanol-d₄) δ 8.31 (d, 1H), 7.95-7.88 (m, 2H), 7.67-7.53 (m, 3H), 7.28 (d, 1H), 6.93-6.86 (m, 3H), 6.35-6.28 (m, 1H), 5.67-5.59 (m, 1H), 3.31-3.20 (m, 1H), 3.06-3.00 (m, 2H), 2.88 (s, 6H), 2.83-2.74 (m, 1H), 2.64-2.55 (m, 1H), 2.06-2.03 (m, 6H), 2.03-2.00 (m, 2H), 1.71-1.63 (m, 1H), 1.47-1.38 (m, 1H), 0.90-0.82 (m, 6H).

### Example 35: Preparation of Compound 35 and Stereoisomer Thereof

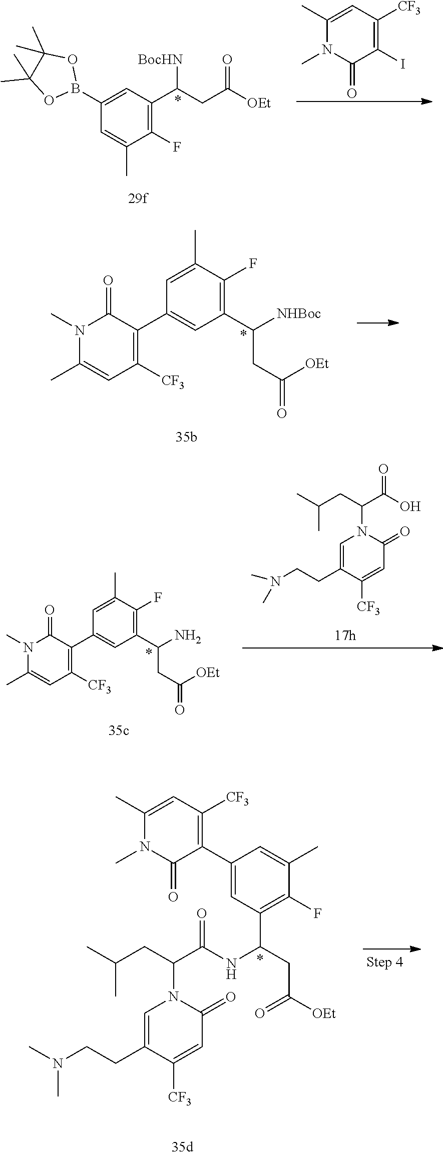 [SMILES: CCOC(=O)CC(NC(=O)OC(C)(C)C)c1cc(B2OC(C)(C)C(C)(C)O2)cc(C)c1F ; Cc1cc(C(F)(F)F)c(I)c(=O)n1C ; CCOC(=O)CC(NC(=O)OC(C)(C)C)c1cc(-c2c(C(F)(F)F)cc(C)n(C)c2=O)cc(C)c1F ; CCOC(=O)CC(N)c1cc(-c2c(C(F)(F)F)cc(C)n(C)c2=O)cc(C)c1F ; CC(C)CC(C(=O)O)n1cc(CCN(C)C)c(C(F)(F)F)cc1=O ; CCOC(=O)CC(NC(=O)C(CC(C)C)n1cc(CCN(C)C)c(C(F)(F)F)cc1=O)c1cc(-c2c(C(F)(F)F)cc(C)n(C)c2=O)cc(C)c1F]

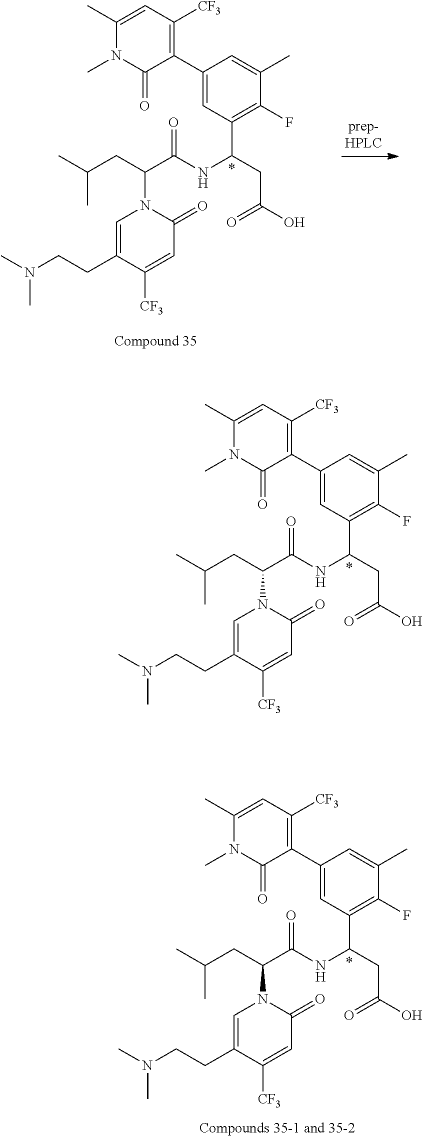

### Step 1: Synthesis of 35b

Under nitrogen protection, 29f (1.5 g, 3.32 mmol), 3-iodo-1,6-dimethyl-4-(trifluoromethyl)pyridin-2(1H)-one (1.26 g, 3.98 mmol) and potassium carbonate (1.38 g, 9.98 mmol) were added to 1,4-dioxane (30.0 mL) and water (3.0 mL), followed by Pd(dppf)Cl₂ (0.24 g, 0.33 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 35b (1 g, yield: 58.54%).

LC-Ms m/z (ESI): 515.5 [M+H]⁺.

### Step 2: Synthesis of 35c

35b (1 g, 1.94 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude 35c.

LC-Ms m/z (ESI): 415.4 [M+H]⁺.

### Step 3: Synthesis of 35d

Under nitrogen protection, 17h (670 mg, 1.93 mmol) was dissolved in dry DMF (1.5 mL), and HATU (1.47 g, 3.86 mmol) and DIPEA (1.0 g, 7.74 mmol) were added. At room temperature, the reaction mixture was stirred for 40 min, and then crude 35c (800 mg, 1.93 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 35d (1 g, yield: 69.57%).

LC-Ms m/z (ESI): 745.7 [M+H]⁺.

### Step 4: Synthesis of Compounds 35-1 and 35-2

35d (1.0 g, 1.34 mmol) was dissolved in THF (6 mL) and water (2 mL), 1,5,7-triazidobicyclo(4.4.0)dec-5-ene (560 mg, 4.02 mmol) was then added, and the resulting mixture was reacted at room temperature for 5 h. The reaction mixture was adjusted with 1 N hydrochloric acid to pH 5-6 and concentrated under reduced pressure to afford crude compound 35, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 35-1 (50 mg, retention time=5.137 min, yield: 5.21%) and compound 35-2 (50 mg, retention time=5.202 min, yield: 5.21%).

Compound 35-1:

LC-Ms m/z (ESI): 717.7 [M+H]⁺.

¹H NMR (400 MHz, DMSO-d₆) δ 9.13 (s, 1H), 7.78 (s, 1H), 6.92 (s, 2H), 6.62 (s, 1H), 6.47 (s, 1H), 5.63-5.51 (m, 1H), 5.40 (s, 1H), 3.48 (s, 3H), 2.68-2.53 (m, 4H), 2.49-2.46 (m, 4H) 2.40-2.28 (m, 2H), 2.20 (s, 3H), 2.16 (s, 6H), 1.83-1.70 (m, 1H), 1.34-1.22 (m, 1H), 0.87 (d, 3H), 0.82 (d, 3H).

Compound 35-2:

LC-Ms m/z (ESI): 717.7 [M+H]⁺.

¹H NMR (400 MHz, DMSO-d₆) δ 9.13 (d, 1H), 7.76 (s, 1H), 7.01-6.93 (m, 2H), 6.74 (s, 1H), 6.51 (s, 1H), 5.63-5.56 (m, 1H), 5.49-5.39 (m, 1H), 3.50 (s, 3H), 2.64-2.54 (m, 4H), 2.50-2.46 (m, 4H), 2.45-2.36 (m, 2H), 2.23 (s, 3H), 2.19 (s, 6H), 1.81-1.70 (m, 1H), 1.25-1.07 (m, 1H), 0.80-0.74 (m, 6H).

### Example 36: Preparation of Compound 36 and Stereoisomer Thereof

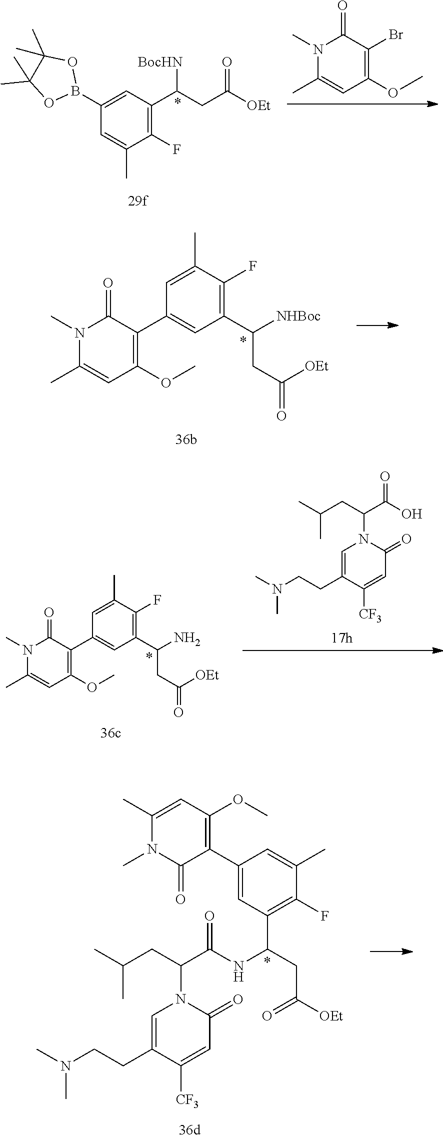

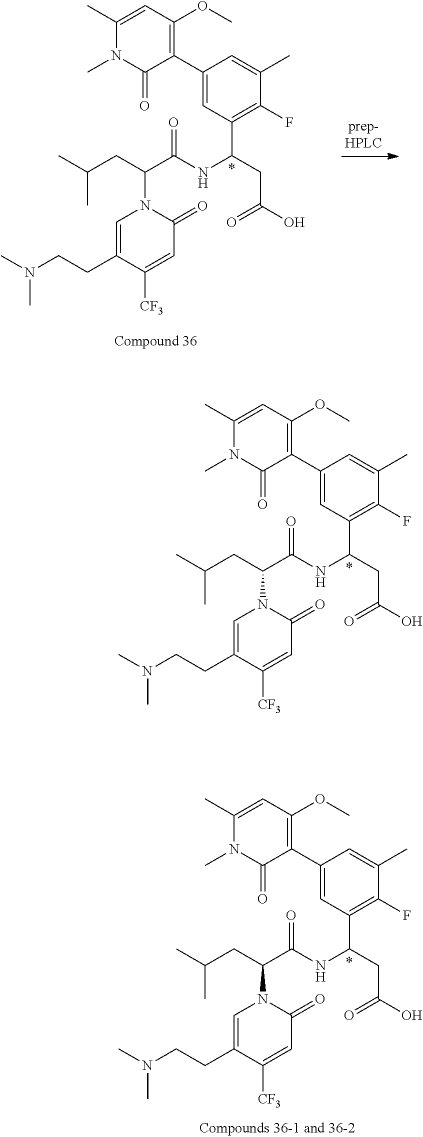

### Step 1: Synthesis of 36b

Under nitrogen protection, 29f (500 mg, 1.11 mmol), 3-bromo-4-methoxy-1,6-dimethylpyridin-2(1H)-one (0.26 g, 1.11 mmol) and potassium carbonate (0.46 g, 3.33 mmol) were added to 1,4-dioxane (30.0 mL) and water (3.0 mL), followed by Pd(dppf)Cl₂ (0.081 g, 0.11 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 36b (0.38 g, yield: 71.84%).

LC-Ms m/z (ESI): 477.5 [M+H]⁺.

### Step 2: Synthesis of 36c

36b (380 mg, 0.80 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude 36c.

LC-Ms m/z (ESI): 377.4 [M+H]⁺.

### Step 3: Synthesis of 36d

Under nitrogen protection, 17h (280 mg, 0.80 mmol) was dissolved in dry DMF (1.5 mL), and HATU (610 mg, 1.61 mmol) and DIPEA (620 mg, 4.80 mmol) were added. At room temperature, the reaction mixture was stirred for 40 min, and then crude 36c (300 mg, 0.80 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to silica gel column chromatography to afford 36d (420 mg, yield: 74.28%).

LC-Ms m/z (ESI): 707.7 [M+H]⁺.

### Step 4: Synthesis of Compounds 36-1 and 36-2

36d (420 mg, 0.59 mmol) was dissolved in THF (6 mL) and water (2 mL), 1,5,7-triazidobicyclo(4.4.0)dec-5-ene (250 mg, 1.77 mmol) was then added, and the resulting mixture was reacted at room temperature for 5 h. The reaction mixture was adjusted with 1 N hydrochloric acid to pH 5-6 and concentrated under reduced pressure to afford crude compound 36, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 36-1 (60 mg, retention time=3.417 min, yield: 14.98%) and compound 36-2 (60 mg, retention time=3.371 min, yield: 14.98%).

Compound 36-1:

LC-Ms m/z (ESI): 680.1[M+H]⁺.

¹H NMR (400 MHz, DMSO-d₆) δ 9.01 (d, 1H), 7.79 (s, 1H), 7.16-7.08 (m, 1H), 7.09-7.00 (m, 1H), 6.74 (s, 1H), 6.32 (s, 1H), 5.67-5.58 (m, 1H), 5.45-5.37 (m, 1H), 3.73 (s, 3H), 3.42 (s, 3H), 2.63-2.52 (m, 4H), 2.43-2.39 (m, 4H), 2.22-2.20 (m, 3H), 2.19 (s, 6H), 1.93-1.84 (m, 1H), 1.80-1.71 (m, 1H), 1.26-1.16 (m, 1H), 0.84-0.77 (m, 6H).

Compound 36-2:

LC-Ms m/z (ESI): 680.1 [M+H]⁺.

### Example 37: Preparation of Compound 37 and Stereoisomer Thereof

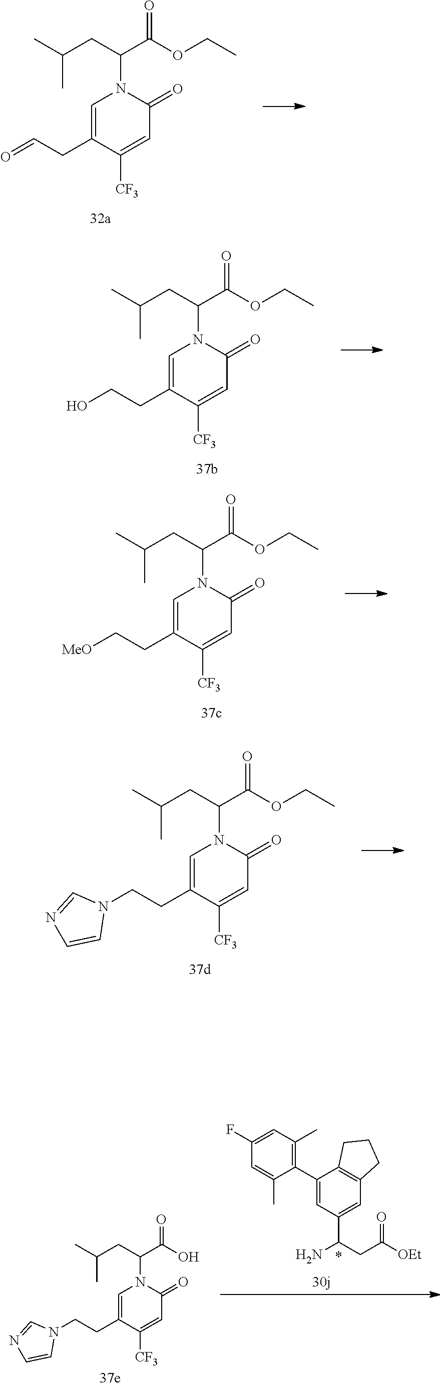 [SMILES: CCOC(=O)C(CC(C)C)n1cc(CC=O)c(C(F)(F)F)cc1=O ; CCOC(=O)C(CC(C)C)n1cc(CCO)c(C(F)(F)F)cc1=O ; CCOC(=O)C(CC(C)C)n1cc(CCOC)c(C(F)(F)F)cc1=O ; CCOC(=O)C(CC(C)C)n1cc(CCn2ccnc2)c(C(F)(F)F)cc1=O ; CC(C)CC(C(=O)O)n1cc(CCn2ccnc2)c(C(F)(F)F)cc1=O ; CCOC(=O)CC(N)c1cc2c(c(-c3c(C)cc(F)cc3C)c1)CCC2]

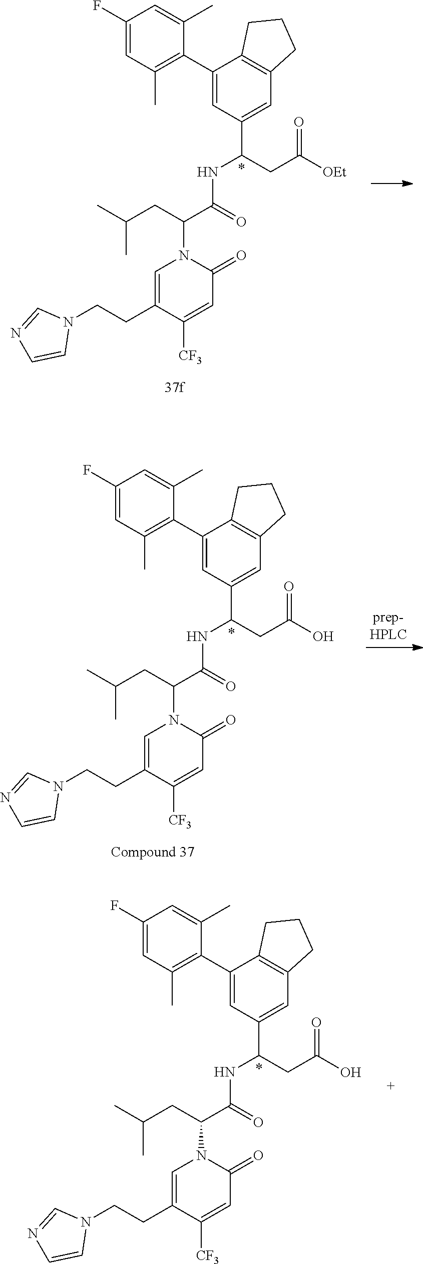 [SMILES: CCOC(=O)CC(NC(=O)C(CC(C)C)n1cc(CCn2ccnc2)c(C(F)(F)F)cc1=O)c1cc2c(c(-c3c(C)cc(F)cc3C)c1)CCC2 ; Cc1cc(F)cc(C)c1-c1cc(C(CC(=O)O)NC(=O)C(CC(C)C)n2cc(CCn3ccnc3)c(C(F)(F)F)cc2=O)cc2c1CCC2 ; Cc1cc(F)cc(C)c1-c1cc(C(CC(=O)O)NC(=O)[C@@H](CC(C)C)n2cc(CCn3ccnc3)c(C(F)(F)F)cc2=O)cc2c1CCC2]

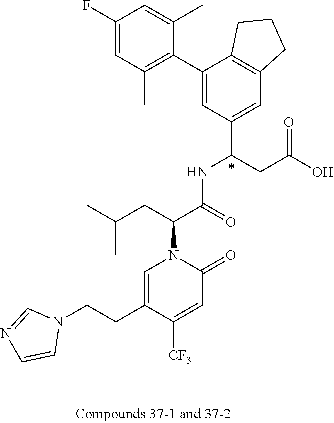 [SMILES: Cc1cc(F)cc(C)c1-c1cc(C(CC(=O)O)NC(=O)[C@H](CC(C)C)n2cc(CCn3ccnc3)c(C(F)(F)F)cc2=O)cc2c1CCC2]

### Step 1: Synthesis of 37b

Under nitrogen protection, 32a (4.2 g, 12.09 mmol) was dissolved in methanol (50 mL). At 0° C., sodium borohydride (0.91 g, 24.06 mmol) was slowly added and the mixture was reacted at 0° C. for 30 min. The reaction mixture was adjusted with 1 N hydrochloric acid solution to about pH 7. The reaction liquid was poured into ice water (100 mL), extracted with dichloromethane (50×2 mL), washed with saturated brine (80 mL), dried over anhydrous sodium sulfate, filtered and concentrated. The residue was subjected to silica gel column chromatography to afford 37b (3.2 g, 75.76%).

LC-Ms m/z (ESI): 350.3 [M+H]⁺.

### Step 2: Synthesis of 37c

Under nitrogen protection, 37b (3.0 g, 8.59 mmol) was dissolved in ultra-dry dichloromethane (100 mL), followed by the addition of triethylamine solution (5.21 g, 51.51 mmol). Under ice bath, methylsulfonyl chloride was added dropwise, and the resulting mixture was reacted at room temperature for 3 h. The reaction liquid was poured into ice water (200 mL), extracted with dichloromethane (100×2 mL), washed with saturated brine (80 mL), dried over anhydrous sodium sulfate, filtered and concentrated. The residue was subjected to silica gel column chromatography to afford 37c (3.0 g, 81.71%).

LC-Ms m/z (ESI): 428.4 [M+H]⁺.

### Step 3: Synthesis of 37d

Under nitrogen protection, 37c (3.0 g, 7.02 mmol) and imidazole (0.95 g, 14.04 mmol) were dissolved in acetonitrile (100 mL), followed by the addition of potassium carbonate (2.91 g, 21.05 mmol), and the resulting mixture was reacted at 80° C. overnight. The reaction liquid was filtered through celite, concentrated under reduced pressure and then subjected to silica gel column chromatography to afford 37d (841 mg, 29.99%).

LC-Ms m/z (ESI): 400.4 [M+H]⁺.

### Step 4: Synthesis of 37e

37d (820 mg, 2.05 mmol) was dissolved in THF (3 mL) and water (1 mL), lithium hydroxide monohydrate (140 mg, 5.97 mmol) was added, and the resulting mixture was reacted at room temperature for 5 h. The reaction mixture was adjusted with 1 N hydrochloric acid to pH 5-6, concentrated under reduced pressure and then purified by reverse phase column to afford compound 37e (720 mg, 94.58%).

LC-Ms m/z (ESI): 372.3 [M+H]⁺.

### Step 4: Synthesis of 37f

Under nitrogen protection, 37e (190 mg, 0.51 mmol) was dissolved in dry DMF (3 mL), and HATU (390 mg, 1.02 mmol) and DIPEA (260 mg, 2.04 mmol) were added. At room temperature, the reaction mixture was stirred for 40 min, and then crude 30j (180 mg, 0.51 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 37f (300 mg, yield: 82.99%).

LC-Ms m/z (ESI): 709.8 [M+H]⁺.

### Step 5: Synthesis of Compounds 37-1 and 37-2

37f (300 mg, 0.42 mmol) was dissolved in THF (3 mL) and water (1 mL), 1,5,7-triazidobicyclo(4.4.0)dec-5-ene (180 mg, 1.29 mmol) was then added, and the resulting mixture was reacted at room temperature for 5 h. The reaction mixture was adjusted with 1 N hydrochloric acid to pH 5-6 and concentrated under reduced pressure to afford crude compound 37, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid)) to afford the trifluoroacetate of compound 37-1 (10 mg, retention time=4.229 min, yield: 3.50%) and trifluoroacetate of compound 37-2 (10 mg, retention time=4.321 min, yield: 3.50%).

Trifluoroacetate of compound 37-1:

LC-Ms m/z (ESI): 681.3 [M+H]⁺.

¹H NMR (400 MHz, Methanol-d₄) δ 9.05 (d, 1H), 8.91 (d, 1H), 7.68 (s, 1H), 7.61 (t, 1H), 7.49 (t, 1H), 7.21 (d, 1H), 6.86-6.73 (m, 4H), 5.75-5.66 (m, 1H), 5.41-5.32 (m, 1H), 4.54-4.39 (m, 2H), 3.20-3.11 (m, 2H), 2.96 (t, 2H), 2.88-2.82 (m, 2H), 2.47-2.39 (m, 2H), 2.08-1.97 (m, 3H), 1.95-1.86 (m, 4H), 1.84 (s, 3H), 1.45-1.35 (m, 1H), 1.02-0.91 (m, 6H).

Trifluoroacetate of compound 37-2:

LC-Ms m/z (ESI): 681.9 [M+H]⁺.

¹H NMR (400 MHz, Methanol-d₄) δ 9.04 (d, 1H), 8.88 (t, 1H), 7.72 (s, 1H), 7.63 (t, 1H), 7.56 (t, 1H), 7.27 (s, 1H), 6.91-6.82 (m, 4H), 5.74-5.67 (m, 1H), 5.43-5.31 (m, 1H), 4.59-4.43 (m, 2H), 3.21-3.10 (m, 2H), 3.00 (t, 2H), 2.92-2.77 (m, 2H), 2.48 (t, 2H), 2.10-2.01 (m, 2H), 1.96 (s, 6H), 1.80-1.69 (m, 2H), 1.35-1.21 (m, 1H), 0.91-0.81 (m, 6H).

### Example 38: Preparation of Compound 38 and Stereoisomer Thereof

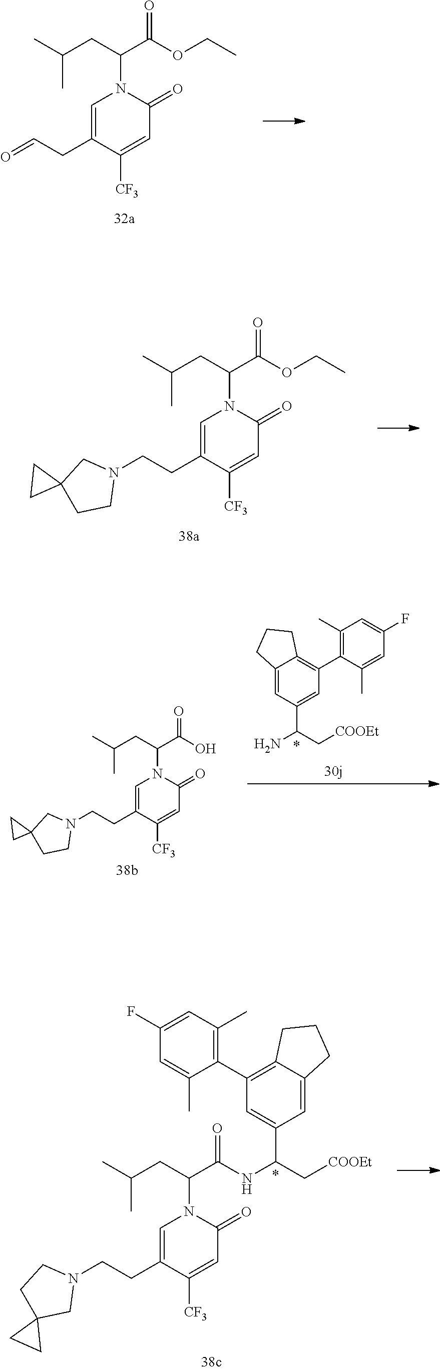 [SMILES: CCOC(=O)C(CC(C)C)n1cc(CC=O)c(C(F)(F)F)cc1=O ; CCOC(=O)C(CC(C)C)n1cc(CCN2CCC3(CC3)C2)c(C(F)(F)F)cc1=O ; CC(C)CC(C(=O)O)n1cc(CCN2CCC3(CC3)C2)c(C(F)(F)F)cc1=O ; CCOC(=O)CC(N)c1cc2c(c(-c3c(C)cc(F)cc3C)c1)CCC2 ; CCOC(=O)CC(NC(=O)C(CC(C)C)n1cc(CCN2CCC3(CC3)C2)c(C(F)(F)F)cc1=O)c1cc2c(c(-c3c(C)cc(F)cc3C)c1)CCC2]

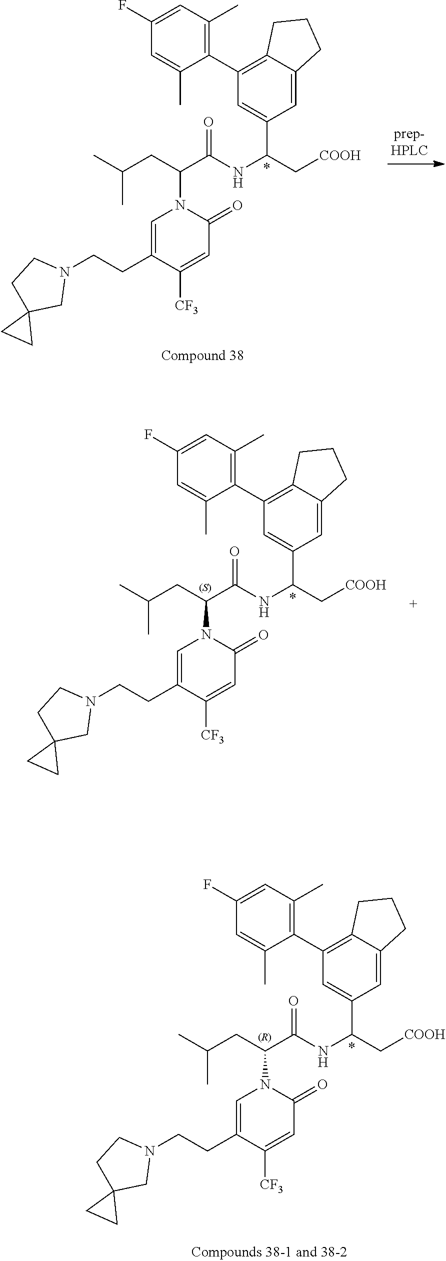

### Step 1: Synthesis of 38a

Under nitrogen protection, 32a (1.5 g, 4.32 mmol) and 5-azaspiro[2.4]heptane (0.42 g, 4.32 mmol) were dissolved in 1,2-dichloroethane (10 mL), acetic acid (0.5 mL) was added dropwise, and the mixture was stirred at room temperature for 2 hours. Sodium triacetoxyborohydride (1.82 g, 8.64 mmol) was added and the resulting mixture was stirred for 16 hours, concentrated under reduced pressure and then subjected to flash column chromatography on a silica gel column to afford 38a (1.0 g, yield: 54.05%).

LC-Ms m/z (ESI): 429.2 [M+H]⁺.

### Step 2: Synthesis of 38b

38a (1.0 g, 2.34 mmol) was dissolved in THF (3 mL) and water (1 mL), lithium hydroxide monohydrate (112 mg, 4.68 mmol) was added, and the resulting mixture was reacted at room temperature for 5 h. The reaction mixture was adjusted with 1 N hydrochloric acid to pH 5-6, concentrated under reduced pressure and then subjected to column chromatography to afford 38b (650 mg, 69.55%).

LC-Ms m/z (ESI): 401.2 [M+H]⁺.

### Step 3: Synthesis of 38c

Under nitrogen protection, 30j (234 mg, 0.66 mmol) was dissolved in dry DMF (1.5 mL), and HATU (376 mg, 0.99 mmol) and DIPEA (225 mg, 1.74 mmol) were added. At room temperature, the reaction mixture was stirred for 40 min, and then crude 38b (264 mg, 0.66 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 38d (470 mg).

LC-Ms m/z (ESI): 738.4 [M+H]⁺.

### Step 4: Synthesis of Compounds 38-1 and 38-2

38d (470 mg) was dissolved in THF (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 38, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 38-1 (15 mg, retention time=5.420 min, yield: 3.20%) and compound 38-2 (15 mg, retention time=5.491 min, yield: 3.20%).

Compound 38-1: LC-Ms m/z (ESI): 710.3 [M+H]⁺.

¹H NMR (400 MHz, Methanol-d₄) δ 7.89 (s, 1H), 7.19 (d, 1H), 6.84-6.76 (m, 4H), 5.73-5.65 (m, 1H), 5.34-5.27 (m, 1H), 3.42 (t, 1H), 3.26 (t, 1H), 3.16 (s, 2H), 2.99-2.87 (m, 4H), 2.77-2.62 (m, 3H), 2.40 (t, 2H), 2.06-1.92 (m, 6H), 1.90 (s, 3H), 1.84 (s, 3H), 1.46-1.37 (m, 1H), 0.95 (d, 3H), 0.94 (d, 3H), 0.79-0.66 (m, 4H).

Compound 38-2: LC-Ms m/z (ESI): 710.3 [M+H]⁺.

¹H NMR (400 MHz, Methanol-d₄) δ 7.81 (s, 1H), 7.22 (s, 1H), 6.89 (s, 1H), 6.84 (s, 1H), 6.82-6.78 (m, 2H), 5.59 (t, 1H), 5.44-5.38 (m, 1H), 3.56-3.49 (m, 2H), 3.49-3.33 (m, 2H), 3.26 (s, 2H), 3.03-2.85 (m, 4H), 2.63-2.50 (m, 2H), 2.44 (t, 2H), 2.06-1.98 (m, 4H), 1.94 (s, 3H), 1.93 (s, 3H), 1.70-1.62 (m, 1H), 1.45-1.36 (m, 1H), 0.90 (d, 3H), 0.89 (d, 3H), 0.83-0.70 (m, 4H).

### Example 39: Preparation of Compound 39 and Stereoisomer Thereof

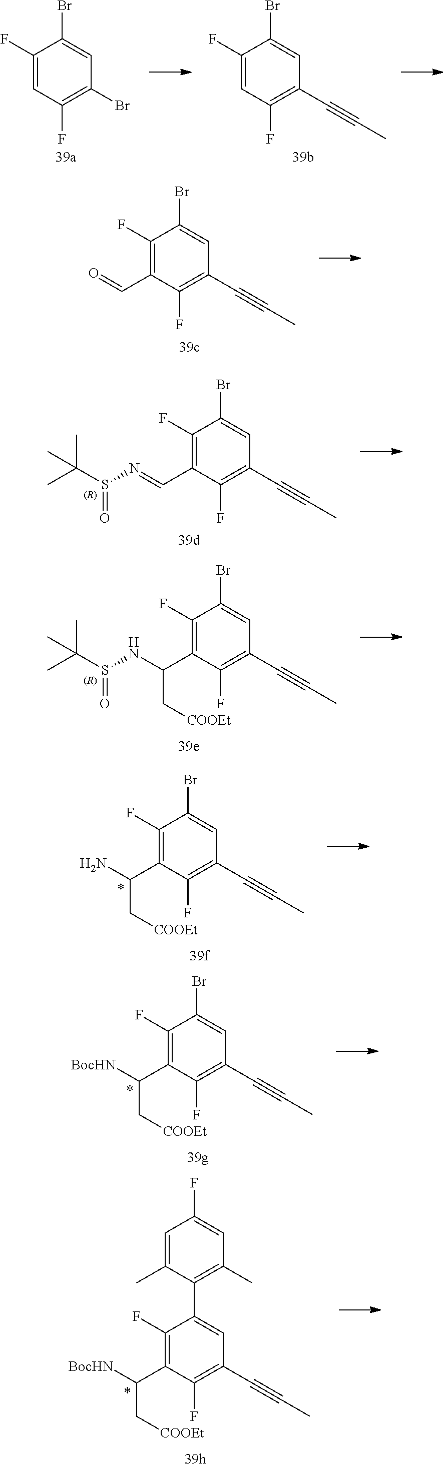

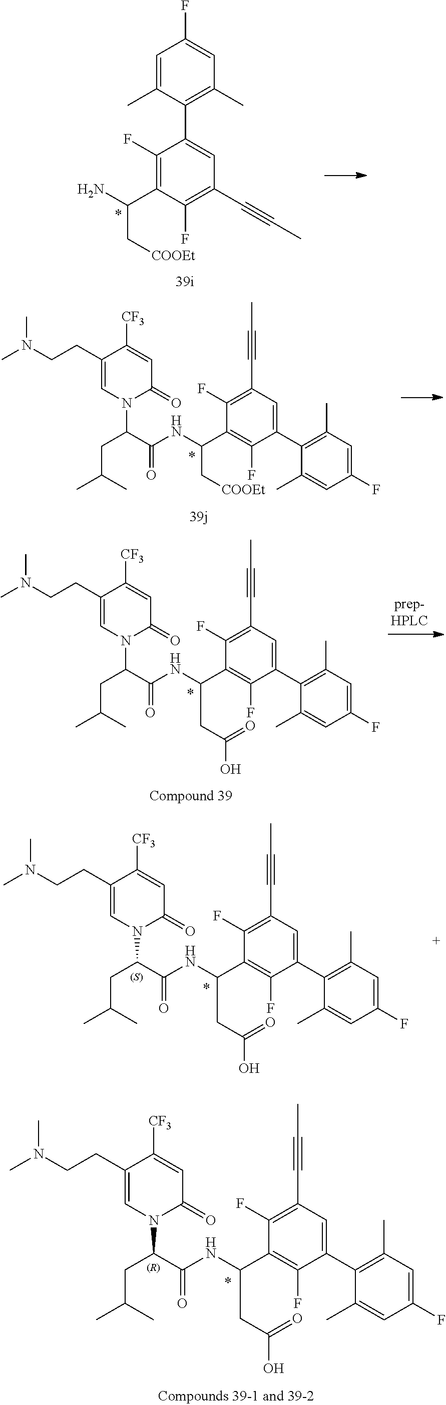

### Step 1: Synthesis of 39b

Under nitrogen protection, 39a (25 g, 91.94 mmol), CuI (3.5 g, 18.39 mmol), PdCl₂(PPh₃)₂ (6.45 g, 9.19 mmol), 1-(trimethylsilyl)propyne (9.29 g, 82.75 mmol) and cesium fluoride (41.90 g, 275.82 mmol) were dissolved in THF (250 mL) and the resulting mixture was reacted at 60° C. for 72 h, concentrated under reduced pressure and then subjected to silica gel column chromatography to afford 39b (8.5 g, 40.02%).

### Step 2: Synthesis of 39c

Under nitrogen protection, 39b (8.5 g, 36.79 mmol) was dissolved in THF (75 mL). At −78° C., lithium diisopropylamide (5.91 g, 55.19 mmol) was added and the resulting mixture was stirred for 1 h. DMF (5.38 g, 73.58 mmol) was added dropwise and the mixture was stirred for 30 min. Saturated aqueous ammonium chloride solution (150 mL) was added and the resulting mixture was extracted with ethyl acetate (200 mL×3). The organic phases were combined, dried over anhydrous sodium sulfate and concentrated to afford 39c (7.2 g, 75.55%).

### Step 3: Synthesis of 39d

Under nitrogen protection, 39c (7.2 g, 27.79 mmol) and R-tert-butylsulfinamide (4.04 g, 33.35 mmol) were dissolved in THF (75 mL), tetraethyl titanate (9.51 g, 41.69 mmol) was slowly added, and the resulting mixture was reacted at 45° C. for 3 h and concentrated under reduced pressure to afford a crude, which was subjected to flash column chromatography on a silica gel column to afford 39d (6.80 g, yield: 67.55%).

### Step 4: Synthesis of 39e

Zinc powder (17.19 g, 262.78 mmol) was added to dry THF (45 mL), the mixture was subjected to nitrogen replacement three times, CuCl (5.57 g, 56.31 mmol) was added, and the resulting mixture was reacted at 60° C. for 2 h. The reaction mixture was cooled to room temperature, ethyl bromoacetate (15.67 g, 93.85 mmol) was slowly added, and the resulting mixture was reacted at 60° C. for 1 h and cooled to 0° C. A solution of 39d (6.8 g, 18.77 mmol) in THF (10 mL) was added and the mixture was stirred at 0° C. for 3 h. The reaction mixture was filtered through celite and saturated ammonium chloride solution (200 mL) was added. The resulting mixture was extracted with ethyl acetate (300 ml×3), washed with saturated brine (200 mL), dried over anhydrous sodium sulfate, filtered and concentrated under reduced pressure. The residue was subjected to flash column chromatography to afford 39e (7.0 g, yield: 82.81%).

### Step 5: Synthesis of 39f

39e (3.0 g, 6.66 mmol) was dissolved in dichloromethane (10 mL), 4 N hydrogen chloride dioxane solution (5 mL) was added, and the resulting mixture was stirred at room temperature for 3 h. The reaction mixture was concentrated under reduced pressure to afford crude 39f.

### Step 6: Synthesis of Compound 39g

Under nitrogen protection, crude 39f (2.0 g, 5.78 mmol) was dissolved in dry dichloromethane (35 mL), triethylamine (1.75 g, 17.34 mmol) and di-tert-butyl dicarbonate (1.51 g, 6.94 mmol) were added, and the resulting mixture was reacted at room temperature for 3 h. The solvent was removed under reduced pressure and the residue was subjected to column chromatography on a silica gel column to afford 39g (1. 3 g, 50.40%).

### Step 7: Synthesis of 39h

Under nitrogen protection, 39g (0.45 g, 1.11 mmol), 2,6-dimethyl-4-fluorophenylboronic acid (0.56 g, 3.33 mmol), potassium phosphate (0.47 g, 2.21 mmol), and RuPhos Pd G3 (CAS: 1445085-77-7) (0.19 g, 0.22 mmol) were dissolved in DMF (8 mL) and water (0.8 mL) and the mixture was stirred at 75° C. for 3 h. Water (20 mL) was added and the resulting mixture was extracted with ethyl acetate (30 ml×3), washed with saturated brine (20 mL×2), dried over anhydrous sodium sulfate, filtered and concentrated under reduced pressure. The residue was subjected to flash column chromatography to afford 39h (0.3 g, yield: 60.39%).

### Step 8: Synthesis of 39i

39h (300 mg, 0.61 mmol) was dissolved in dichloromethane (2 mL), 4 N hydrogen chloride dioxane solution (2 mL) was added, and the resulting mixture was stirred at room temperature for 3 h. The reaction mixture was concentrated under reduced pressure to afford crude 39i.

LC-Ms m/z (ESI): 390.2 [M+H]⁺.

### Step 9: Synthesis of 39j

Under nitrogen protection, 17h (180 mg, 0.51 mmol) was dissolved in dry DMF (1.5 mL), and HATU (290 mg, 0.77 mmol) and DIPEA (130 mg, 1.0 mmol) were added. At room temperature, the reaction mixture was stirred for 40 min, and then crude 39i (200 mg, 0.51 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 39j (200 mg, yield: 54.48%).

LC-Ms m/z (ESI): 720.3 [M+H]⁺.

### Step 10: Synthesis of Compounds 39-1 and 39-2

39j (200 mg, 0.28 mmol) was dissolved in methanol (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 39, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 39-1 (20 mg, retention time=4.149 min, yield: 10.33%) and compound 39-2 (20 mg, retention time=4.226 min, yield: 10.33%).

Compound 39-1: LC-Ms m/z (ESI): 692.3 [M+H]⁺.

¹H NMR (400 MHz, Methanol-d₄) δ 7.86-7.81 (m, 1H), 7.04 (t, 1H), 6.89-6.80 (m, 3H), 5.74-5.62 (m, 2H), 3.11-3.04 (m, 2H), 2.98-2.87 (m, 3H), 2.76-2.66 (m, 7H), 2.03 (s, 3H), 2.00 (s, 3H), 1.99-1.94 (m, 2H), 1.92 (s, 3H), 1.42-1.31 (m, 1H), 0.96-0.90 (m, 5H).

Compound 39-2: LC-Ms m/z (ESI): 692.3 [M+H]⁺.

¹H NMR (400 MHz, Methanol-d₄) δ 7.79 (s, 1H), 7.10 (t, 1H), 6.96-6.79 (m, 3H), 5.82-5.75 (m, 1H), 5.62 (t, 1H), 3.26-3.11 (m, 2H), 3.00-2.92 (m, 2H), 2.83-2.73 (m, 7H), 2.61-2.52 (m, 1H), 2.06 (s, 3H), 2.04-1.98 (m, 6H), 1.94-1.85 (m, 1H), 1.79-1.68 (m, 1H), 1.39-1.27 (m, 1H), 0.92-0.84 (m, 6H).

### Example 40: Preparation of Compound 40 and Stereoisomer Thereof

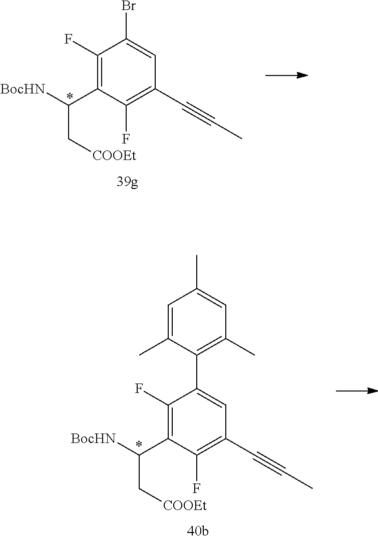 [SMILES: CC#Cc1cc(Br)c(F)c(C(CC(=O)OCC)NC(=O)OC(C)(C)C)c1F ; CC#Cc1cc(-c2c(C)cc(C)cc2C)c(F)c(C(CC(=O)OCC)NC(=O)OC(C)(C)C)c1F]

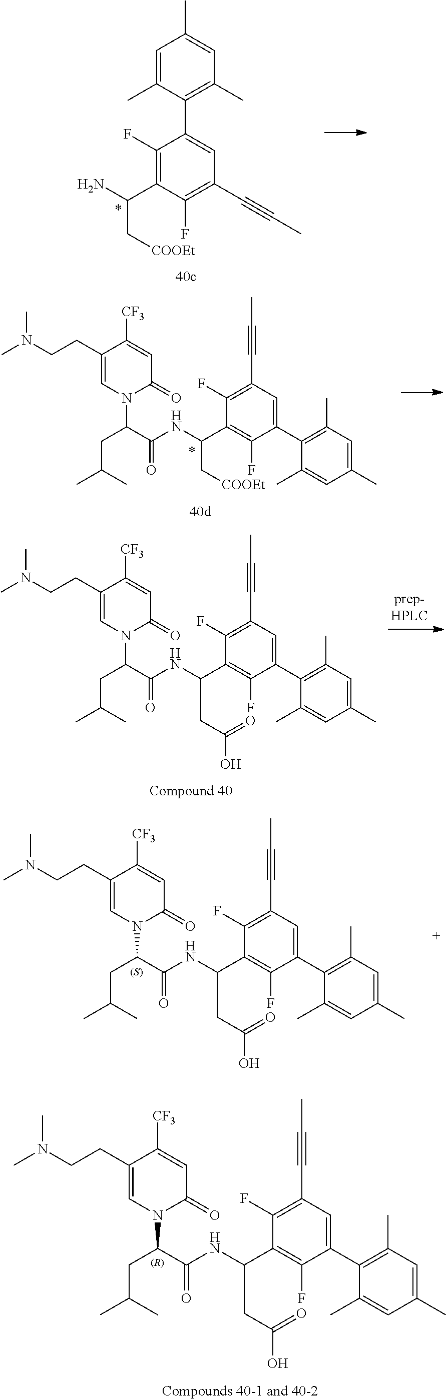

### Step 1: Synthesis of 40b

Under nitrogen protection, 39g (0.45 g, 1.11 mmol), 2,4,6-trimethyl-phenylboronic acid (0.56 g, 3.33 mmol), potassium phosphate (0.47 g, 2.21 mmol), and RuPhos Pd G3 (0.19 g, 0.22 mmol) were dissolved in DMF (8 mL) and water (0.8 mL) and the mixture was stirred at 75° C. for 3 h. Water (20 mL) was added and the resulting mixture was extracted with ethyl acetate (30 ml×3), washed with saturated brine (20 mL×2), dried over anhydrous sodium sulfate, filtered and concentrated under reduced pressure. The residue was subjected to flash column chromatography to afford 40b (0.3 g, yield: 60.39%).

### Step 2: Synthesis of 40c

40b (300 mg, 0.61 mmol) was dissolved in dichloromethane (2 mL), 4 N hydrogen chloride dioxane solution (2 mL) was added, and the resulting mixture was stirred at room temperature for 3 h. The reaction mixture was concentrated under reduced pressure to afford crude 40c.

LC-Ms m/z (ESI): 386.2 [M+H]⁺.

### Step 3: Synthesis of 40d

Under nitrogen protection, 17h (180 mg, 0.52 mmol) was dissolved in dry DMF (1.5 mL), and HATU (290 mg, 0.78 mmol) and DIPEA (130 mg, 1.04 mmol) were added. At room temperature, the reaction mixture was stirred for 40 min, and then crude 40c (200 mg, 0.51 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 40d (200 mg, yield: 53.73%).

LC-Ms m/z (ESI): 716.3 [M+H]⁺.

### Step 4: Synthesis of Compounds 40-1 and 40-2

40d (200 mg, 0.28 mmol) was dissolved in methanol (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 40, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 40-1 (20 mg, retention time=4.226 min, yield: 10.39%) and compound 40-2 (20 mg, retention time=5.699 min, yield: 10.39%).

Compound 40-1: LC-Ms m/z (ESI): 688.3 [M+H]⁺.

¹H NMR (400 MHz, Methanol-d₄) δ 7.83 (s, 1H), 7.01 (t, 1H), 6.92-6.87 (m, 2H), 6.84 (s, 1H), 5.72-5.63 (m, 2H), 3.10-3.02 (m, 2H), 2.96-2.86 (m, 3H), 2.73 (s, 6H), 2.71-2.65 (m, 1H), 2.28 (s, 3H), 2.03 (s, 3H), 1.99-1.92 (m, 5H), 1.86 (s, 3H), 1.45-1.34 (m, 1H), 0.97-0.89 (m, 6H).

Compound 40-2: LC-Ms m/z (ESI): 688.3 [M+H]⁺.

¹H NMR (400 MHz, Methanol-d₄) δ 7.79 (s, 1H), 7.07 (t, 1H), 6.96-6.88 (m, 3H), 5.83-5.76 (m, 1H), 5.62 (t, 1H), 3.29-3.12 (m, 2H), 3.01-2.94 (m, 2H), 2.84-2.74 (m, 7H), 2.60-2.53 (m, 1H), 2.29 (s, 3H), 2.06 (s, 3H), 2.00-1.95 (m, 6H), 1.94-1.87 (m, 1H), 1.79-1.68 (m, 1H), 1.41-1.27 (m, 1H), 0.93-0.84 (m, 6H).

### Example 41: Preparation of Compound 41 and Stereoisomer Thereof

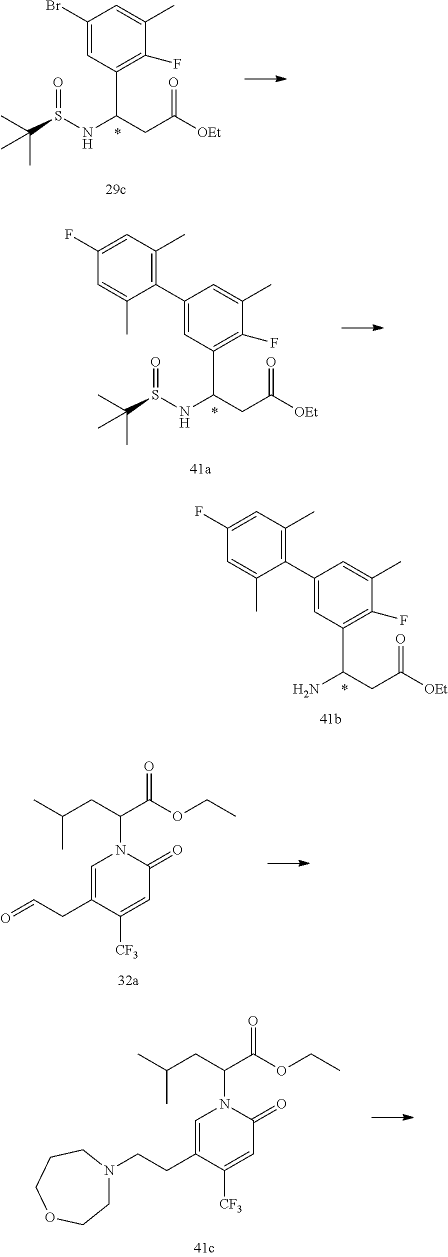 [SMILES: CCOC(=O)CC(N[S@](=O)C(C)(C)C)c1cc(Br)cc(C)c1F ; CCOC(=O)CC(N[S@](=O)C(C)(C)C)c1cc(-c2c(C)cc(F)cc2C)cc(C)c1F ; CCOC(=O)CC(N)c1cc(-c2c(C)cc(F)cc2C)cc(C)c1F ; CCOC(=O)C(CC(C)C)n1cc(CC=O)c(C(F)(F)F)cc1=O ; CCOC(=O)C(CC(C)C)n1cc(CCN2CCCOCC2)c(C(F)(F)F)cc1=O]

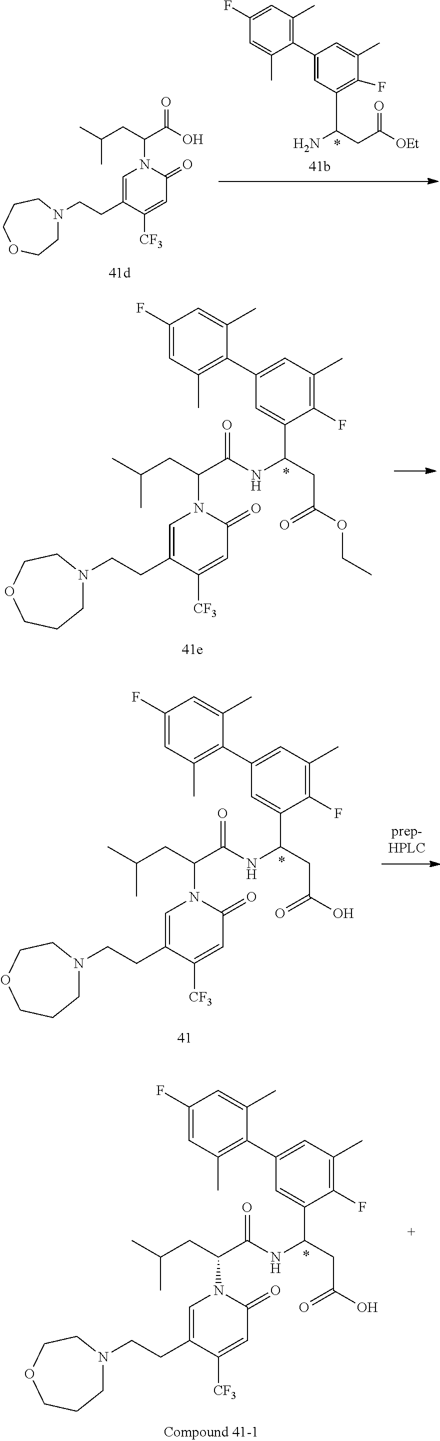 [SMILES: CC(C)CC(C(=O)O)n1cc(CCN2CCCOCC2)c(C(F)(F)F)cc1=O ; CCOC(=O)CC(N)c1cc(-c2c(C)cc(F)cc2C)cc(C)c1F ; CCOC(=O)CC(NC(=O)C(CC(C)C)n1cc(CCN2CCCOCC2)c(C(F)(F)F)cc1=O)c1cc(-c2c(C)cc(F)cc2C)cc(C)c1F ; Cc1cc(-c2c(C)cc(F)cc2C)cc(C(CC(=O)O)NC(=O)C(CC(C)C)n2cc(CCN3CCCOCC3)c(C(F)(F)F)cc2=O)c1F ; Cc1cc(-c2c(C)cc(F)cc2C)cc(C(CC(=O)O)NC(=O)[C@@H](CC(C)C)n2cc(CCN3CCCOCC3)c(C(F)(F)F)cc2=O)c1F]

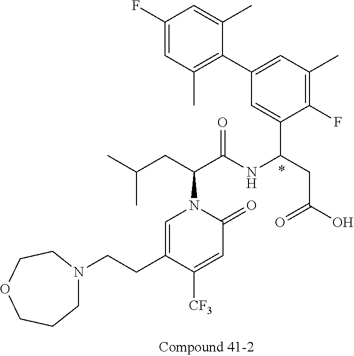 [SMILES: Cc1cc(-c2c(C)cc(F)cc2C)cc(C(CC(=O)O)NC(=O)[C@H](CC(C)C)n2cc(CCN3CCCOCC3)c(C(F)(F)F)cc2=O)c1F]

### Step 1: Synthesis of 41a

Under nitrogen protection, 29c (315 mg, 0.77 mmol), 4-fluoro2,6-dimethylphenylboronic acid (194 mg, 1.16 mmol) and cesium carbonate (753 mg, 2.31 mmol) were added to 1,4-dioxane (3.5 mL) and water (0.35 mL), followed by Pd(dppf)Cl₂ (122 mg, 0.15 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 41a (250 mg, yield: 71.90%).

### Step 2: Synthesis of 41b

41a (250 mg, 0.55 mmol) was dissolved in THF (3 mL), 4 N hydrogen chloride dioxane solution (1.5 mL) was added, and the resulting mixture was stirred at room temperature for 3 h. The reaction mixture was concentrated under reduced pressure to afford crude 41b.

### Step 3: Synthesis of 41c

Under nitrogen protection, 1,4-oxazepane hydrochloride (360 mg, 2.59 mmol) was dissolved in 1,2-dichloroethane (8 mL), DIPEA was added, and the resulting mixture was reacted at room temperature for 15 min. 32a (0.9 g, 2.59 mmol) and acetic acid (0.15 mL) were added and the mixture was stirred at room temperature for 1 h. Sodium triacetoxyborohydride (820 mg, 3.86 mmol) was added and the mixture was stirred for 16 h and concentrated under reduced pressure to afford a crude, which was subjected to flash column chromatography on a silica gel column to afford 41c (0.49 g, yield: 43.75%).

LC-Ms m/z (ESI): 433.7 [M+H]⁺.

### Step 4: Synthesis of 41d

41c (0.72 g, 1.66 mmol) was dissolved in ethanol (6 mL) and water (0.6 mL), lithium hydroxide monohydrate (210 mg, 4.98 mmol) was added, and the resulting mixture was reacted at room temperature for 3 h. The reaction mixture was adjusted with 1 N hydrochloric acid to pH 4, concentrated under reduced pressure and then subjected to column chromatography to afford 41d (530 mg, yield: 78.95%).

LC-Ms m/z (ESI): 405.6 [M+H]⁺.

### Step 5: Synthesis of 41e

Under nitrogen protection, 41d (340 mg, 0.84 mmol) and 41b (290 mg, 0.84 mmol) were dissolved in dry DMF (10 mL), and HATU (480 mg, 1.26 mmol) and DIPEA (220 mg, 1.68 mmol) were added. The reaction mixture was reacted at room temperature for 3 h, diluted with water, and extracted with ethyl acetate. The organic phases were combined, dried over anhydrous sodium sulfate, filtered and concentrated under reduced pressure. The residue was subjected to column chromatography on a silica gel column to afford 41e (347 mg, yield: 56.29%).

LC-Ms m/z (ESI): 735.0 [M+H]⁺.

### Step 6: Synthesis of Compounds 41-1 and 41-2

41e (310 mg, 0.42 mmol) was dissolved in THF (3 mL) and water (2 mL), lithium hydroxide monohydrate (35 mg, 0.84 mmol) was added, and the resulting mixture was reacted at room temperature for 5 h. The reaction mixture was concentrated under reduced pressure to afford crude compound 41, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 41-1 (50 mg, retention time=5.420 min, yield: 16.87%) and compound 41-2 (50 mg, retention time=5.491 min, yield: 16.87%).

Compound 41-1:

LC-Ms m/z (ESI): 706.3 [M+H]⁺.

¹H NMR (400 MHz, DMSO-d₆) δ 9.66 (s, 1H), 9.04 (d, 1H), 7.93 (s, 1H), 7.00-6.88 (m, 4H), 6.69 (s, 1H), 5.62-5.56 (m 1H), 5.49-5.41 (m, 1H), 3.92-3.52 (m, 6H), 3.26-3.18 (m, 3H), 2.97-2.82 (m, 2H), 2.75-2.63 (m, 2H), 2.26-2.20 (m, 3H), 2.12-2.03 (m, 2H), 1.97-1.92 (m, 4H), 1.84-1.79 (m, 1H), 1.77 (s, 3H), 1.39-1.28 (m, 1H), 0.89 (d, 3H), 0.85 (d, 3H).

Compound 41-2:

LC-Ms m/z (ESI): 706.3 [M+H]⁺.

1H NMR (400 MHz, DMSO-d₆) δ 9.60 (s, 1H), 9.16 (d, 1H), 7.97 (s, 1H), 7.02-6.94 (m, 4H), 6.82 (s, 1H), 5.68-5.59 (m, 1H), 5.54-5.45 (m, 1H), 3.93-3.49 (m, 6H), 3.28-3.17 (m, 3H), 2.98-2.84 (m, 2H), 2.72-2.66 (m, 2H), 2.26 (s, 3H), 2.16-2.02 (m, 2H), 1.99-1.90 (m, 6H), 1.83-1.72 (m, 1H), 1.65-1.55 (m, 1H), 1.22-1.13 (m, 1H), 0.80-0.73 (m, 6H).

### Example 42: Preparation of Compound 42 and Stereoisomer Thereof

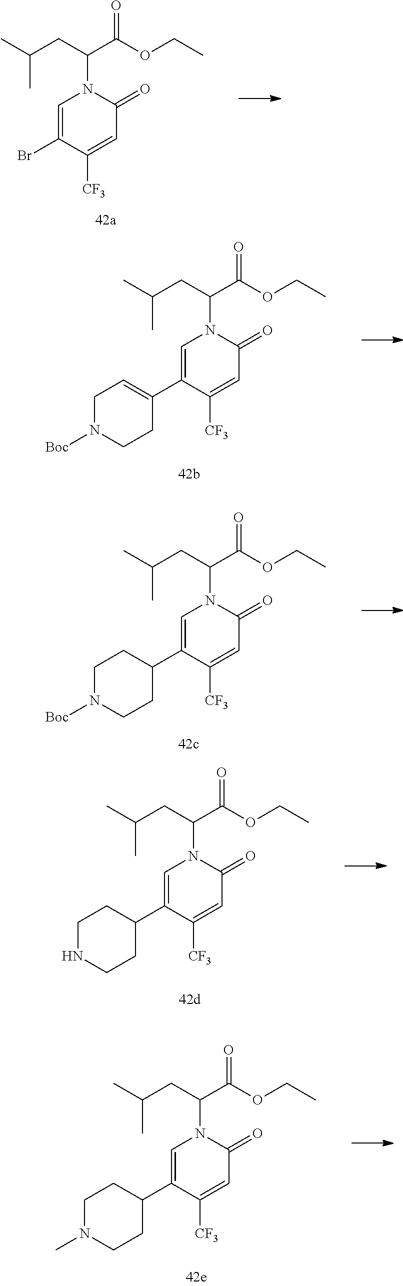

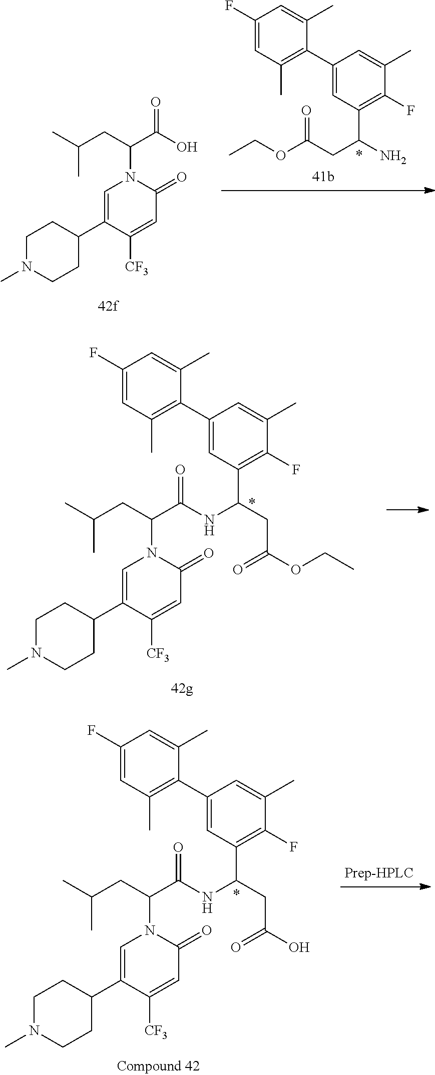

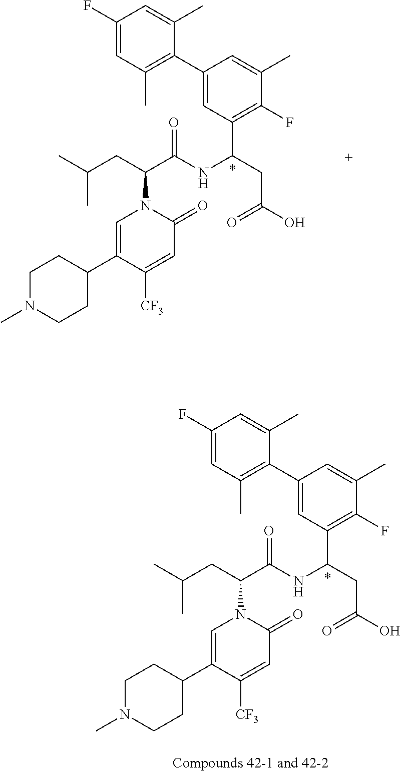 [SMILES: Cc1cc(-c2c(C)cc(F)cc2C)cc(C(CC(=O)O)NC(=O)[C@H](CC(C)C)n2cc(C3CCN(C)CC3)c(C(F)(F)F)cc2=O)c1F ; Cc1cc(-c2c(C)cc(F)cc2C)cc(C(CC(=O)O)NC(=O)[C@@H](CC(C)C)n2cc(C3CCN(C)CC3)c(C(F)(F)F)cc2=O)c1F]

### Step 1: Synthesis of 42b

Under nitrogen protection, 42a (5 g, 13.0 mmol), 3,6-dihydro-2H-pyridine-1-tertbutoxycarbonyl-4-boronic acid pinacol ester (4.84 g, 15.6 mmol) and cesium carbonate (12.8 g, 39.1 mmol) were added to 1,4-dioxane (30.0 mL) and water (3.0 mL), followed by Pd(PPh₃)₄ (1.51 g, 1.3 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 42b (4.6 g, yield: 73.1%).

LC-Ms m/z (ESI): 487.2 [M+H]⁺.

### Step 2: Synthesis of 42c

42b (4.6 g, 9.4 mmol) was dissolved in methanol (100 mL), Pd/C (460 mg) was added, and the mixture was reacted at 25° C. for 4 h. The reaction mixture was concentrated under reduced pressure, adjusted with saturated sodium bicarbonate to about pH 8 and subjected to silica gel column chromatography to afford 42c (2.4 g, 52.1%).

LC-Ms m/z (ESI): 489.3 [M+H]⁺.

### Step 3: Synthesis of 42d

42c (2.4 g, 4.91 mmol) was dissolved in dichloromethane (15 mL), TFA (5 mL) was added, and the resulting mixture was reacted at 25° C. for 2 h. The reaction mixture was concentrated under reduced pressure. Ethyl acetate (150 mL) was added and the resulting mixture was successively washed with water (30 mL) and saturated brine (30 mL), and dried over anhydrous sodium sulfate to afford 42d (1.5 g, 78.9%).

LC-Ms m/z (ESI): 389.3 [M+H]⁺.

### Step 4: Synthesis of 42e

Under nitrogen protection, 42d (1.5 g, 3.86 mmol) and carbaldehyde aqueous solution (3 mL) were dissolved in 1,2-dichloroethane (10 mL), acetic acid (0.5 mL) was added dropwise, and the mixture was stirred at room temperature for 2 h. Sodium triacetoxyborohydride (1.62 g, 7.72 mmol) was added and the resulting mixture was stirred for 16 h, and concentrated under reduced pressure to afford a crude, which was subjected to flash column chromatography on a silica gel column to afford 42e (1.3 g, yield: 83.8%).

LC-Ms m/z (ESI): 403.2 [M+H]⁺.

### Step 5: Synthesis of 42f

42d (300 mg, 0.74 mmol) was dissolved in THF (3 mL) and water (1 mL), lithium hydroxide monohydrate (90 mg, 2.2 mmol) was added, and the resulting mixture was reacted at room temperature for 3 h. The reaction mixture was adjusted with 1 N hydrochloric acid to pH 5-6 and concentrated under reduced pressure to afford crude 42F.

LC-Ms m/z (ESI): 375.4 [M+H]⁺.

### Step 6: Synthesis of Compound 42g

Under nitrogen protection, 42f (250 mg, 0.67 mmol) was dissolved in dry DMF (5 mL), and HATU (304 mg, 0.8 mmol) and DIPEA (259 mg, 2.01 mmol) were added. At room temperature, the reaction mixture was stirred for 10 min, and then crude 41b (278 mg, 0.8 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to silica gel column chromatography to afford 42g (245 mg, yield: 52.3%).

LC-Ms m/z (ESI): 704.3 [M+H]⁺.

### Step 7: Synthesis of 42

42g (245 mg, 0.35 mmol) was dissolved in THF (5 mL) and water (1 mL), lithium hydroxide monohydrate (44 mg, 1.04 mmol) was added, and the resulting mixture was reacted at room temperature for 4 h. The reaction mixture was adjusted with 1 N hydrochloric acid to pH 5-6 and concentrated under reduced pressure to afford crude compound 42, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 42-1 (31 mg, retention time=5.324 min) and compound 42-2 (42 mg, retention time=5.501 min).

Compound 42-1:

¹H NMR (400 MHz, DMSO-d₆) δ 9.14 (d, 1H), 7.78 (s, 1H), 7.01-6.93 (m, 4H), 6.74 (s, 1H), 5.65-5.56 (m, 1H), 5.52-5.42 (m, 1H), 2.93-2.82 (m, 2H), 2.69-2.57 (m, 2H), 2.45-2.31 (m, 1H), 2.26 (s, 3H), 2.26-2.14 (m, 3H), 2.00-1.91 (m, 7H), 1.82-1.71 (m, 1H), 1.70-1.65 (m, 5H), 1.19-1.05 (m, 1H), 0.78-0.67 (m, 6H).

LC-Ms m/z (ESI): 676.3 [M+H]⁺.

Compound 42-2:

¹H NMR (400 MHz, DMSO-d₆) δ 9.14 (d, 1H), 7.78 (s, 1H), 7.02-6.88 (m, 4H), 6.74 (s, 1H), 5.66-5.56 (m, 1H), 5.52-5.43 (m, 1H), 2.93-2.82 (m, 2H), 2.69-2.60 (m, 2H), 2.45-2.31 (m, 1H), 2.26 (s, 3H), 2.20 (s, 3H), 1.98-1.90 (m, 7H), 1.84-1.75 (m, 1H), 1.70-1.57 (m, 5H), 1.21-1.11 (m, 1H), 0.78-0.70 (m, 6H).

LC-Ms m/z (ESI): 676.3 [M+H]⁺.

### Example 43: Preparation of Compound 43 and Stereoisomer Thereof

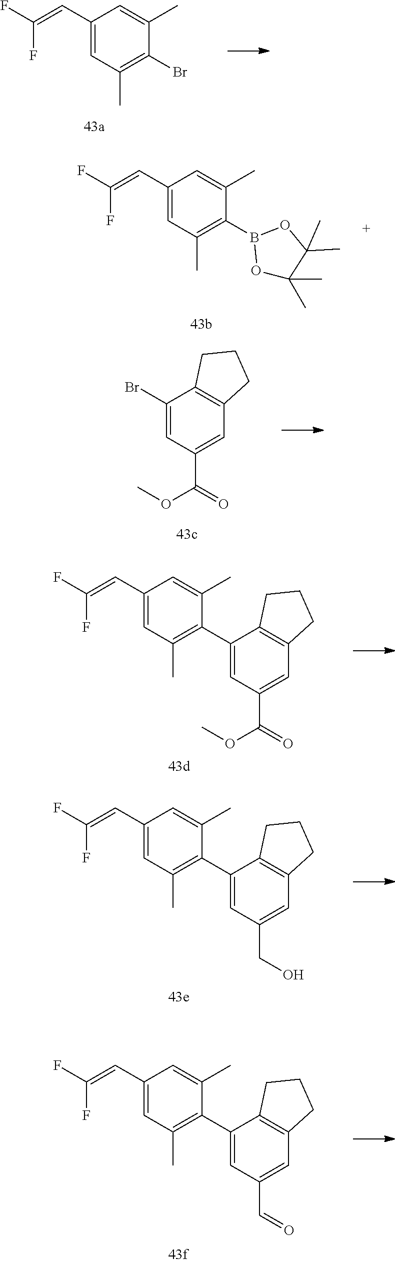 [SMILES: Cc1cc(C=C(F)F)cc(C)c1Br ; Cc1cc(C=C(F)F)cc(C)c1B1OC(C)(C)C(C)(C)O1 ; COC(=O)c1cc(Br)c2c(c1)CCC2 ; COC(=O)c1cc2c(c(-c3c(C)cc(C=C(F)F)cc3C)c1)CCC2 ; Cc1cc(C=C(F)F)cc(C)c1-c1cc(CO)cc2c1CCC2 ; Cc1cc(C=C(F)F)cc(C)c1-c1cc(C=O)cc2c1CCC2]

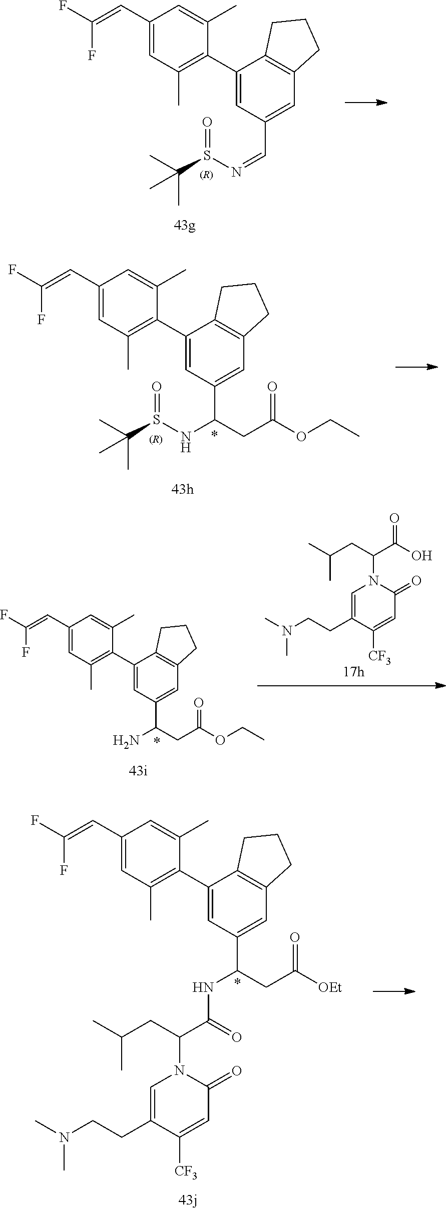

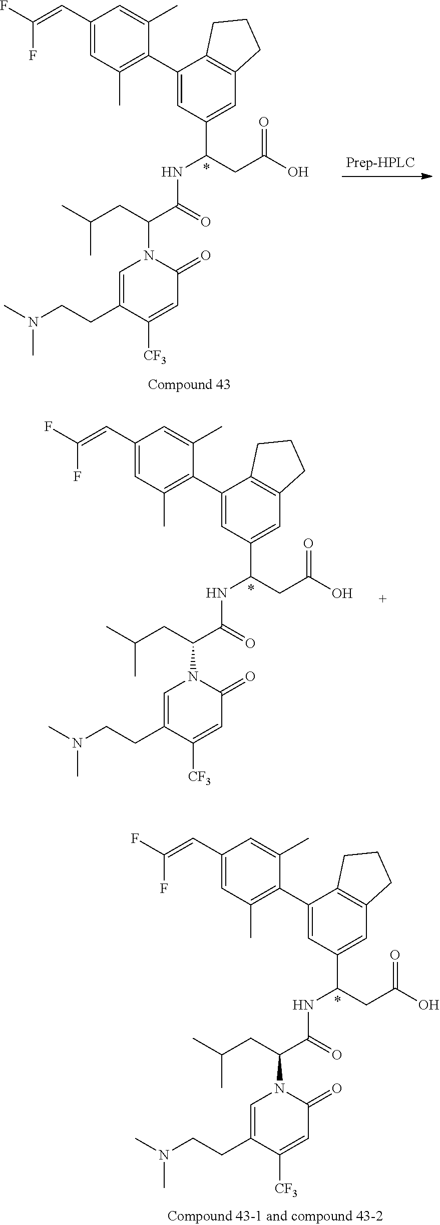 [SMILES: Cc1cc(C=C(F)F)cc(C)c1-c1cc(C(CC(=O)O)NC(=O)C(CC(C)C)n2cc(CCN(C)C)c(C(F)(F)F)cc2=O)cc2c1CCC2 ; Cc1cc(C=C(F)F)cc(C)c1-c1cc(C(CC(=O)O)NC(=O)[C@@H](CC(C)C)n2cc(CCN(C)C)c(C(F)(F)F)cc2=O)cc2c1CCC2 ; Cc1cc(C=C(F)F)cc(C)c1-c1cc(C(CC(=O)O)NC(=O)[C@H](CC(C)C)n2cc(CCN(C)C)c(C(F)(F)F)cc2=O)cc2c1CCC2]

### Step 1: Synthesis of 43b

43a (1.0 g, 4.05 mmol, see Journal of Fluorine Chemistry, 2013, 150, 53-59 for the synthesis), Pd(dppf)C₂ (0.23 g, 0.41 mmol), potassium carbonate (0.99 g, 10.13 mmol) and bis(pinacolato)diboron (1.54 g, 6.07 mmol) were dissolved in dioxane (5 ml) and the mixture was reacted at 100° C. under nitrogen atmosphere for 4 h. The reaction mixture was concentrated under reduced pressure and then separated by silica gel column chromatography to afford 43b (0.55 g, 74.53%).

### Step 2: Synthesis of 43d

43b (208 mg, 0.71 mmol), 43c (150 mg, 0.59 mmol) and cesium carbonate (576 mg, 1.77 mmol) were added to 1,4-dioxane (3.5 mL) and water (0.35 mL), followed by Pd(PPh₃)₄ (136 mg, 0.12 mmol), and the mixture was reacted at 100° C. under nitrogen atmosphere in a sealed tube reaction for 5 h. The reaction system was cooled to room temperature and filtered. Ethyl acetate (40 mL) was added and the mixture was successively washed with water (20 mL) and saturated brine (20 mL), dried over anhydrous sodium sulfate, filtered and concentrated under reduced pressure. The residue was subjected to flash chromatography on a silica gel column to afford 43d (340 mg, crude).

### Step 3: Synthesis of 43e

Under nitrogen atmosphere, crude 43d (340 mg, 0.99 mmol) was dissolved in dry dichloromethane (14 mL), the mixture was cooled to −78° C., and diisopropyl aluminum hydride (141 mg, 0.99 mmol) was slowly added. The mixture was stirred at −78° C. for 1 h, and the reaction was quenched at −78° C. by adding ammonium chloride solution (20 mL). The reaction mixture was slowly warmed to room temperature, extracted with dichloromethane (20 mL×3), dried over anhydrous sodium sulfate and filtered. The solvent was removed under reduced pressure and the residue was subjected to column chromatography on a silica gel column to afford 43e (176 mg, two-step yield: 56.92%).

### Step 4: Synthesis of 43f

Under nitrogen atmosphere, crude 43e (176 mg, 0.56 mmol) was dissolved in dry dichloromethane (5 mL), sodium bicarbonate (94 mg, 1.12 mmol) and Dess-Martin periodinane (308 mg, 0.73 mmol) were added at room temperature, and the resulting mixture was reacted at room temperature for 5 h. Saturated sodium thiosulfate solution (5 mL) and saturated sodium bicarbonate solution (5 mL) were added and vigorously stirred to clarification and the mixture was extracted with dichloromethane (20 mL×3), dried over anhydrous sodium sulfate, filtered and concentrated under reduced pressure. The residue was separated and purified by column chromatography on a silica gel column to afford 43f (165 mg, 94.33%).

### Step 5: Synthesis of 43g

Under nitrogen protection, 43f (165 mg, 0.53 mmol) and R-tert-butylsulfinamide (96 mg, 0.80 mmol) were dissolved in tetrahydrofuran (5 mL), tetraethyl titanate (181 mg, 0.80 mmol) was slowly added, and the resulting mixture was reacted at 45° C. for 15 h and concentrated under reduced pressure to afford a crude, which was subjected to flash column chromatography on a silica gel column to afford 43g (210 mg, yield: 95.35%).

LC-Ms m/z (ESI): 416.3 [M+H]⁺.

### Step 6: Synthesis of 43h

Zinc powder (274 mg, 4.2 mmol) was added to dry tetrahydrofuran (3 mL), the mixture was subjected to nitrogen replacement three times, CuCl (89 mg, 0.90 mmol) was added, and the resulting mixture was reacted at 60° C. for 2 h. The reaction mixture was cooled to room temperature, ethyl bromoacetate (250 mg, 1.5 mmol) was slowly added, and the resulting mixture was reacted at 60° C. for 1 h. The reaction liquid was cooled to 0° C., a solution of 43g (125 mg, 0.3 mmol) in tetrahydrofuran (1 mL) was added, and the mixture was stirred at 0° C. for 3 h. The reaction mixture was filtered through celite and saturated ammonium chloride solution (20 mL) was added. The resulting mixture was extracted with ethyl acetate (30 ml×3), washed with saturated brine (20 mL), dried over anhydrous sodium sulfate, filtered and concentrated under reduced pressure. The residue was subjected to flash column chromatography to afford 43h (100 mg, yield: 66.18%).

LC-Ms m/z (ESI): 505.1 [M+H]⁺.

### Step 7: Synthesis of 43i

43h (150 mg, 0.30 mmol) was dissolved in tetrahydrofuran (2 mL), 4 N hydrogen chloride dioxane solution (1.5 mL) was added, and the resulting mixture was stirred at room temperature for 3 h. The reaction mixture was concentrated under reduced pressure to afford the hydrochloride of crude 43i (103 mg).

LC-Ms m/z (ESI): 400.3 [M+H]⁺.

### Step 8: Synthesis of Compound 43j

Under nitrogen protection, 17h (104 mg, 0.30 mmol) was dissolved in dry DMF (1.5 mL), and HATU (228 mg, 0.60 mmol) and DIPEA (232 mg, 1.80 mmol) were added. At room temperature, the reaction mixture was stirred for 40 min, and then crude 43i (103 mg, 0.29 mmol) was added. The resulting mixture was reacted at room temperature overnight. Ethyl acetate (60 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 43j (360 mg).

LC-Ms m/z (ESI): 730.3 [M+H]⁺.

### Step 9: Synthesis of Compounds 43-1 and 43-2

43j was dissolved in tetrahydrofuran (3 mL) and water (1 mL), lithium hydroxide monohydrate (43 mg, 1.02 mmol) was added, and the resulting mixture was reacted at room temperature for 5 h. The reaction mixture was adjusted with 1 N hydrochloric acid to pH 5-6 and concentrated under reduced pressure to afford crude compound 43, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 43-1 (23 mg, retention time=4.378 min, yield: 9.64%) and compound 43-2 (23 mg, retention time=4.463 min, yield: 9.64%).

Compound 43-1:

LC-Ms m/z (ESI): 702.3 [M+H]⁺.

¹H NMR (400 MHz, CD₃OD) δ 7.87 (s, 1H), 7.18 (s, 1H), 7.11-7.03 (m, 2H), 6.79 (s, 1H), 6.76 (s, 1H), 5.70-5.64 (m, 1H), 5.45-5.36 (m, 1H), 5.32-5.27 (m, 1H), 3.07-2.99 (m, 2H), 2.95-2.86 (m, 4H), 2.74-2.67 (m, 7H), 2.44-2.37 (m, 2H), 2.03-1.93 (m, 5H), 1.89 (s, 3H), 1.82 (s, 3H), 1.47-1.38 (m, 1H), 0.98-0.91 (m, 6H).

Compound 43-2:

LC-Ms m/z (ESI): 702.3 [M+H]⁺

¹H NMR (400 MHz, CD₃OD) δ 7.81 (s, 1H), 7.21 (s, 1H), 7.11-7.08 (m, 2H), 6.89 (s, 1H), 6.82-6.80 (m, 1H), 5.63-5.56 (m, 1H), 5.49-5.37 (m, 2H), 3.26-3.14 (m, 2H), 3.01-2.93 (m, 4H), 2.79 (s, 6H), 2.66-2.43 (m, 4H), 2.08-1.95 (m, 3H), 1.94-1.90 (m, 6H), 1.75-1.66 (m, 1H), 1.45-1.37 (m, 1H), 0.93-0.85 (m, 6H).

### Example 44: Preparation of Compound 44 and Stereoisomer Thereof

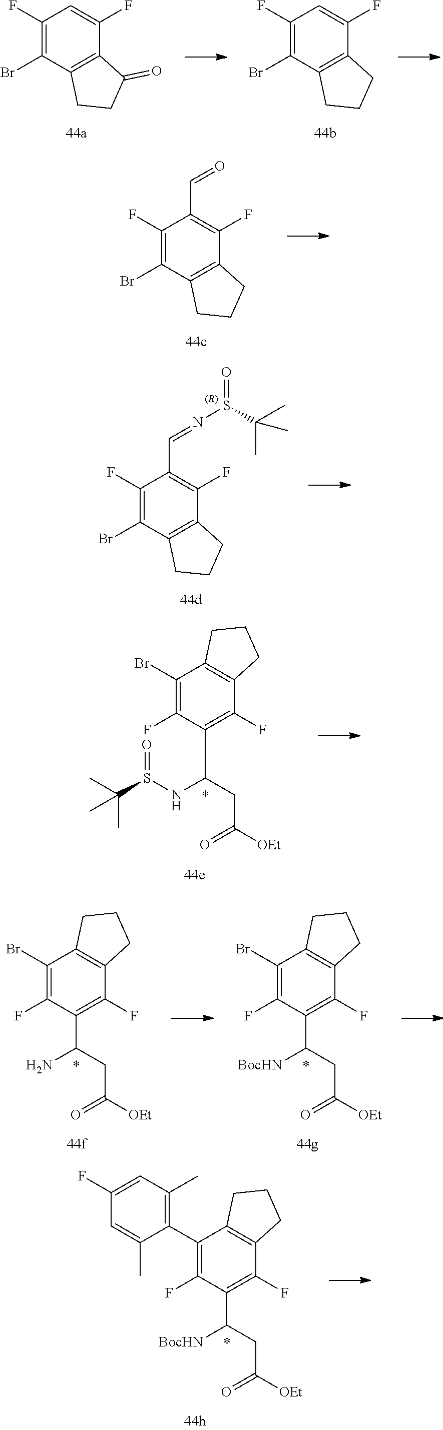

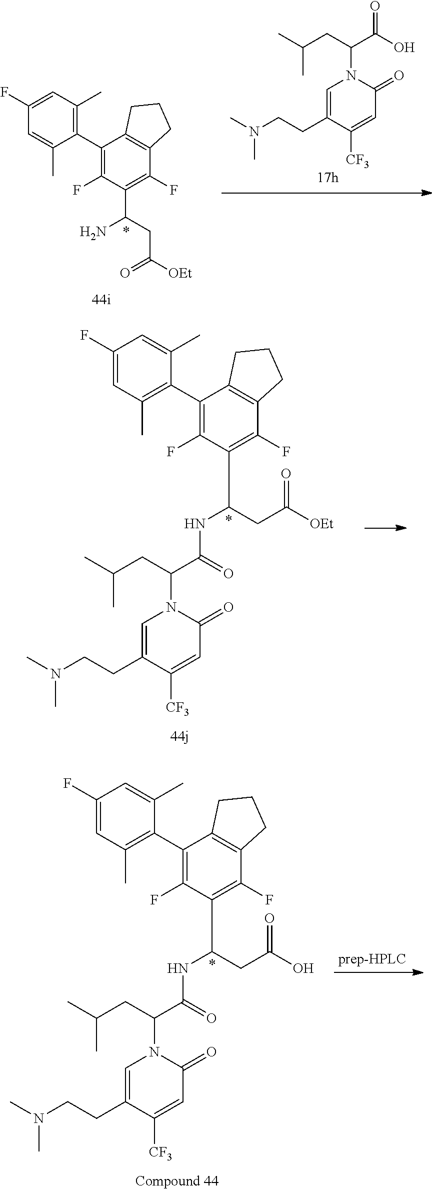 [SMILES: CCOC(=O)CC(N)c1c(F)c2c(c(-c3c(C)cc(F)cc3C)c1F)CCC2 ; CC(C)CC(C(=O)O)n1cc(CCN(C)C)c(C(F)(F)F)cc1=O ; CCOC(=O)CC(NC(=O)C(CC(C)C)n1cc(CCN(C)C)c(C(F)(F)F)cc1=O)c1c(F)c2c(c(-c3c(C)cc(F)cc3C)c1F)CCC2 ; Cc1cc(F)cc(C)c1-c1c(F)c(C(CC(=O)O)NC(=O)C(CC(C)C)n2cc(CCN(C)C)c(C(F)(F)F)cc2=O)c(F)c2c1CCC2]

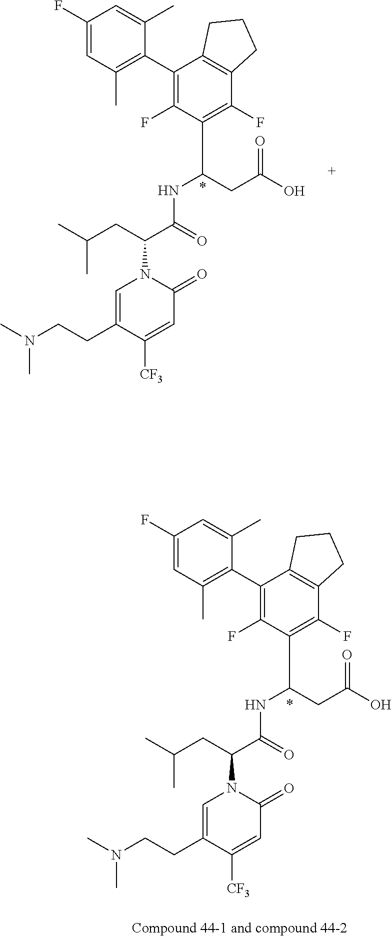 [SMILES: Cc1cc(F)cc(C)c1-c1c(F)c(C(CC(=O)O)NC(=O)[C@@H](CC(C)C)n2cc(CCN(C)C)c(C(F)(F)F)cc2=O)c(F)c2c1CCC2 ; Cc1cc(F)cc(C)c1-c1c(F)c(C(CC(=O)O)NC(=O)[C@H](CC(C)C)n2cc(CCN(C)C)c(C(F)(F)F)cc2=O)c(F)c2c1CCC2]

### Step 1: Synthesis of 44b

44a (1720 mg, 6.96 mmol, see the document US20150148347 for the synthetic route) was dissolved in trifluoroacetic acid (25 mL), triethylsilane (25 ml) was added, and the mixture was reacted at room temperature for 48 h. Triethylsilane (15 mL) was added and the resulting mixture was stirred overnight. The reaction mixture was concentrated under reduced pressure and then subjected to column chromatography on a silica gel column to afford crude 44b (1700 mg).

¹H NMR (400 MHz, CDCl₃) δ 6.61 (t, 1H), 2.99-2.84 (m, 4H), 2.15-2.02 (in, 2H).

### Step 2: Synthesis of 44c

Under nitrogen protection, compound 44b (1700 mg, 7.29 mmol) was dissolved in dry THF (70 mL), the mixture was cooled to −78° C., and lithium diisopropylamide (1.56 g, 14.58 mmol) was slowly added. The mixture was stirred at −78° C. for 0.5 h and dry DMF (2.6 g, 36.45 mmol) was added. At −78° C., the mixture was stirred for another 1 h, and the reaction was quenched with ammonium chloride solution (50 mL). The reaction mixture was extracted with ethyl acetate (80 mL×3), dried over anhydrous sodium sulfate, filtered and concentrated under reduced pressure and the residue was subjected to silica gel column chromatography to afford 44c (1.58 g, two-step yield: 83.02%).

### Step 3: Synthesis of 44d

Under nitrogen protection, 44c (1580 mg, 6.05 mmol) and R-tert-butylsulfinamide (1100 mg, 9.07 mmol) were dissolved in tetrahydrofuran (35 mL), tetraethyl titanate (2070 mg, 9.07 mmol) was slowly added, and the resulting mixture was reacted at 45° C. for 15 h and concentrated under reduced pressure to afford a crude, which was subjected to flash column chromatography on a silica gel column to afford 44d (2100 mg, yield: 95.29%).

LC-Ms m/z (ESI): 364.2[M+H]⁺.

### Step 4: Synthesis of 44e

Zinc powder (1.2 g, 18.34 mmol) was added to dry tetrahydrofuran (8 mL), the mixture was subjected to nitrogen replacement three times, CuCl (389 mg, 3.93 mmol) was added, and the resulting mixture was reacted at 60° C. for 2 h. The reaction mixture was cooled to room temperature, ethyl bromoacetate (1.09 g, 6.55 mmol) was slowly added, and the mixture was reacted at 60° C. for 1 h and then cooled to 0° C. A solution of 44d (478 mg, 1.31 mmol) in tetrahydrofuran (3 mL) was added and the resulting mixture was stirred at 0° C. for 3 h and filtered. Saturated ammonium chloride solution (30 mL) was added and the mixture was extracted with ethyl acetate (40 ml×3), washed with saturated brine (20 mL), dried over anhydrous sodium sulfate, filtered and concentrated under reduced pressure. The residue was subjected to column chromatography to afford 44e (498 mg, yield: 84.04%).

LC-Ms m/z (ESI): 452.4[M+H]⁺.

### Step 5: Synthesis of 44f

44e (448 mg, 0.99 mmol) was dissolved in dichloromethane (3 mL), 4 N hydrogen chloride dioxane solution (2.5 mL) was added, and the resulting mixture was stirred at room temperature for 3 h. The reaction mixture was concentrated under reduced pressure to afford the hydrochloride of crude 44f.

LC-Ms m/z (ESI): 348.3[M+H]⁺.

### Step 6: Synthesis of 44g

The hydrochloride of crude 44f in the above-mentioned step was dissolved in tetrahydrofuran (4 mL) and water (4 mL), sodium carbonate (210 mg, 1.98 mmol) and Boc₂O (240 mg, 1.09 mmol) were added, and the mixture was stirred at room temperature for 3 h. Water (10 mL) was added and the resulting mixture was extracted with ethyl acetate (20 mL×3), dried over anhydrous sodium sulfate, filtered and concentrated under reduced pressure. The residue was subjected to flash column chromatography to afford 44g (420 mg, two-step yield: 86.31%).

LC-Ms m/z (ESI): 350.0 [M+H−Boc]⁺.

### Step 7: Synthesis of 44h

Under nitrogen protection, 44g (230 mg, 0.51 mmol), 2,6-dimethyl-4-fluorophenylboronic acid (128 mg, 0.77 mmol) and potassium phosphate (325 mg, 1.53 mmol) were added to 1,4-dioxane (2.5 mL) and water (0.25 mL), followed by Xphos-G2-Pd (40 mg, 0.051 mmol), and the resulting mixture was reacted at 100° C. in a sealed tube for 24 h. The reaction system was cooled to room temperature. Ethyl acetate (40 mL) was added and the mixture was successively washed with water (20 mL) and saturated brine (20 mL), dried over anhydrous sodium sulfate, filtered and concentrated under reduced pressure. The residue was subjected to flash chromatography on a silica gel column to afford 44h (210 mg, yield: 83.77%).

LC-Ms m/z (ESI): 392.6 [M+H−Boc]⁺.

### Step 8: Synthesis of 44i

44h (210 mg, 0.43 mmol) was dissolved in tetrahydrofuran (3 mL), 4 N hydrogen chloride dioxane solution (2.5 mL) was added, and the resulting mixture was stirred at room temperature for 3 h. The reaction mixture was concentrated under reduced pressure to afford the hydrochloride of crude 44i.

LC-Ms m/z (ESI): 392.6[M+H]⁺.

### Step 9: Synthesis of 44j

Under nitrogen protection, the hydrochloride of crude 44i and 17h (143 mg, 0.41 mmol) were dissolved in dry DMF (3.0 mL), and HOBT (110 mg, 0.82 mmol), EDCI (160 mg, 0.82 mmol) and DIPEA (158 mg, 1.23 mmol) were added. The reaction mixture was reacted at room temperature overnight. Ethyl acetate (60 mL) was added and the resulting mixture was successively washed with water (20 mL×2) and saturated brine (20 mL×1), dried over anhydrous sodium sulfate and filtered. The filtrate was concentrated under reduced pressure and the residue was subjected to column chromatography on a silica gel column to afford 44j (270 mg).

LC-Ms m/z (ESI): 722.3 [M+H]⁺.

### Step 10: Synthesis of Compounds 44-1 and 44-2

44j (270 mg) was dissolved in tetrahydrofuran (4 mL) and water (1 mL), lithium hydroxide monohydrate (44 mg, 1.05 mmol) was added, and the resulting mixture was reacted at room temperature for 5 h. The reaction mixture was adjusted with 1 N hydrochloric acid to pH 5-6 and concentrated under reduced pressure to afford crude compound 44, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 44-1 (23 mg, retention time=4.160 min, two-step yield: 12.36%) and compound 44-2 (30 mg, retention time=4.233 min, two-step yield: 13.18%).

Compound 44-1:

LC-Ms m/z (ESI): 694.3 [M+H]⁺.

¹H NMR (400 MHz, CD₃OD) δ 7.85 (s, 1H), 6.86 (s, 1H), 6.85-6.82 (m, 2H), 5.74-5.63 (m, 2H), 3.15-3.00 (m, 2H), 2.98-2.87 (m, 5H), 2.72 (s, 6H), 2.71-2.64 (m, 1H), 2.48-2.42 (m, 2H), 2.10-2.02 (m, 2H), 1.99-1.94 (m, 5H), 1.89 (s, 3H), 1.46-1.36 (m, 1H), 0.98-0.87 (m, 6H).

Compound 44-2:

LC-Ms m/z (ESI): 694.3 [M+H]⁺.

¹H NMR (400 MHz, CD₃OD) δ 7.79 (s, 1H), 6.91 (s, 1H), 6.89 (s, 1H), 6.86 (s, 1H), 5.86-5.77 (m, 1H), 5.67-5.58 (m, 1H), 3.27-3.11 (m, 2H), 3.04-2.93 (m, 4H), 2.84-2.71 (m, 7H), 2.58-2.45 (m, 3H), 2.14-2.04 (m, 2H), 1.98 (s, 3H), 1.97 (s, 3H), 1.95-1.87 (m, 1H), 1.79-1.70 (m, 1H), 1.40-1.31 (m, 1H), 0.90 (d, 3H), 0.87 (d, 3H).

### Example 45: Preparation of Compound 45 and Stereoisomer Thereof

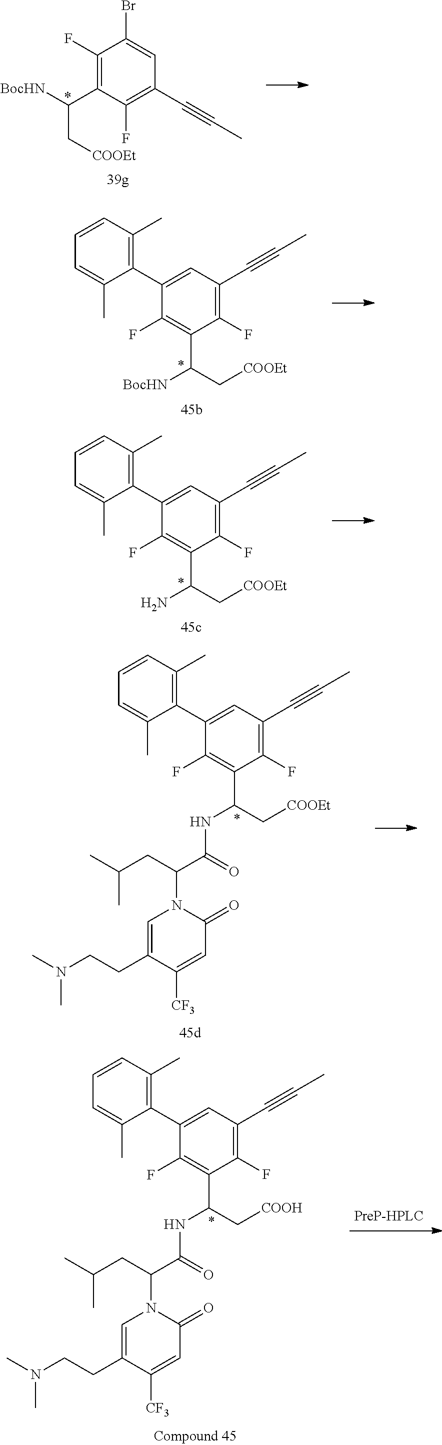

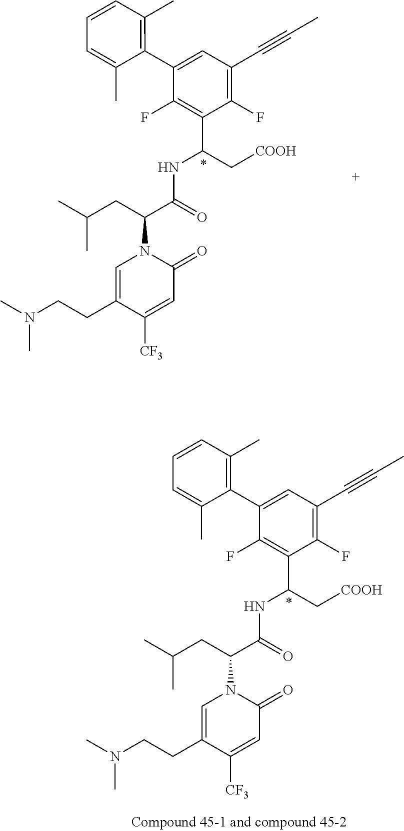

### Step 1: Synthesis of 45b

Under nitrogen protection, 39g (0.45 g, 1.11 mmol), 2,6-dimethylphenylboronic acid (0.50 g, 3.33 mmol), potassium phosphate (0.47 g, 2.21 mmol), and RuPhos Pd G3 (0.19 g, 0.22 mmol) were dissolved in DMF (8 mL) and water (0.8 mL) and the mixture was stirred at 75° C. for 3 h. Water (20 mL) was added and the resulting mixture was extracted with ethyl acetate (30 ml×3), washed with saturated brine (20 mL×2), dried over anhydrous sodium sulfate, filtered and concentrated under reduced pressure. The residue was subjected to flash column chromatography to afford 45b (0.3 g, yield: 57.38%).

### Step 2: Synthesis of 45c

45b (300 mg, 0.64 mmol) was dissolved in dichloromethane (2 mL), 4 N hydrogen chloride dioxane solution (2 mL) was added, and the resulting mixture was stirred at room temperature for 3 h. The reaction mixture was concentrated under reduced pressure to afford crude 45c.

LC-Ms m/z (ESI): 372.2 [M+H]⁺.

### Step 3: Synthesis of 45d

Under nitrogen protection, 17h (180 mg, 0.52 mmol) was dissolved in dry DMF (1.5 mL), and HATU (290 mg, 0.78 mmol) and DIPEA (130 mg, 1.04 mmol) were added. At room temperature, the reaction mixture was stirred for 40 min, and then crude 45c (200 mg, 0.51 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 45d (200 mg, yield: 54.86%).

LC-Ms m/z (ESI): 702.3 [M+H]⁺.

### Step 4: Synthesis of Compounds 45-1 and 45-2

45d (200 mg, 0.28 mmol) was dissolved in methanol (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 45, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 45-1 (20 mg, retention time=4.069 min, yield: 10.61%) and compound 45-2 (20 mg, retention time=4.155 min, yield: 10.61%).

Compound 45-1:

LC-Ms m/z (ESI): 674.3 [M+H]⁺.

¹H NMR (400 MHz, Methanol-d₄) δ 7.84 (s, 1H), 7.20-7.13 (m, 1H), 7.11-7.00 (m, 3H), 6.84 (s, 1H), 5.73-5.64 (m, 2H), 3.11-3.03 (m, 2H), 2.97-2.87 (m, 3H), 2.75-2.65 (m, 7H), 2.04 (s, 3H), 1.99 (s, 3H), 1.98-1.93 (m, 2H), 1.90 (s, 3H), 1.44-1.36 (m, 1H), 0.97-0.89 (m, 6H).

Compound 45-2:

LC-Ms m/z (ESI): 674.3 [M+H]⁺.

¹H NMR (400 MHz, Methanol-d₄) δ 7.80 (s, 1H), 7.21-7.15 (m, 1H), 7.13-7.06 (m, 3H), 6.91 (s, 1H), 5.84-5.78 (m, 1H), 5.66-5.60 (m, 1H), 3.28-3.12 (m, 2H), 3.01-2.94 (m, 2H), 2.84-2.75 (m, 7H), 2.62-2.54 (m, 1H), 2.06 (s, 3H), 2.02 (s, 3H), 2.01 (s, 3H), 1.94-1.87 (m, 1H), 1.78-1.70 (m, 1H), 1.40-1.27 (m, 1H), 0.93-0.85 (m, 6H).

### Example 46: Preparation of Compound 46 and Stereoisomer Thereof

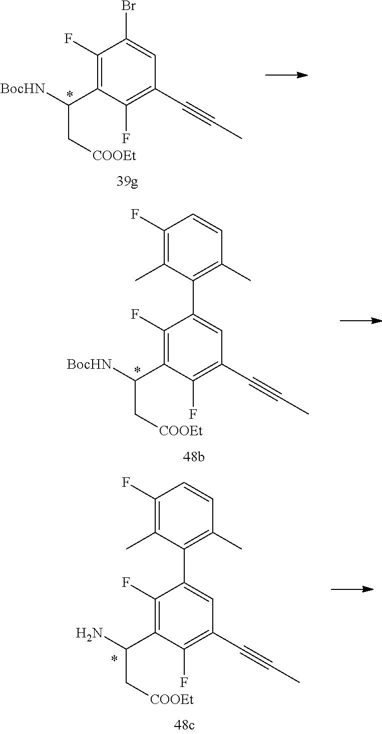 [SMILES: CC#Cc1cc(Br)c(F)c(C(CC(=O)OCC)NC(=O)OC(C)(C)C)c1F ; CC#Cc1cc(-c2c(C)ccc(F)c2C)c(F)c(C(CC(=O)OCC)NC(=O)OC(C)(C)C)c1F ; CC#Cc1cc(-c2c(C)ccc(F)c2C)c(F)c(C(N)CC(=O)OCC)c1F]

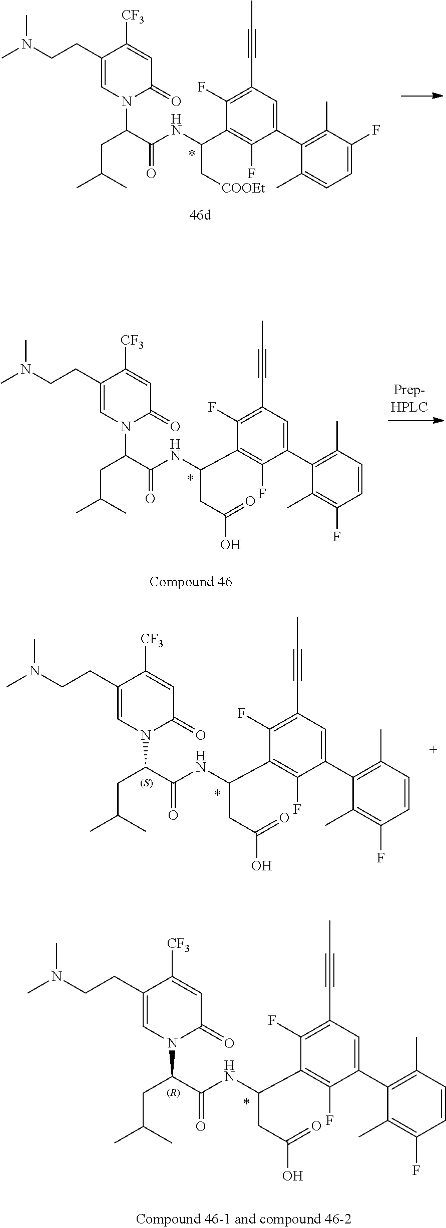

### Step 1: Synthesis of 46b

Under nitrogen protection, 39g (0.45 g, 1.11 mmol), 2,6-dimethyl-3-fluorophenylboronic acid (0.56 g, 3.33 mmol), potassium phosphate (0.47 g, 2.21 mmol), and RuPhos Pd G3 (0.19 g, 0.22 mmol) were dissolved in DMF (8 mL) and water (0.8 mL) and the mixture was stirred at 75° C. for 3 h. Water (20 mL) was added and the resulting mixture was extracted with ethyl acetate (30 mL×3), washed with saturated brine (20 mL×2), dried over anhydrous sodium sulfate, filtered and concentrated under reduced pressure. The residue was subjected to flash column chromatography to afford 46b (0.3 g, yield: 60.39%).

### Step 2: Synthesis of 46c

46b (300 mg, 0.61 mmol) was dissolved in dichloromethane (2 mL), 4 N hydrogen chloride dioxane solution (2 mL) was added, and the resulting mixture was stirred at room temperature for 3 h. The reaction mixture was concentrated under reduced pressure to afford the hydrochloride of crude 46c.

LC-Ms m/z (ESI): 390.2 [M+H]⁺.

### Step 3: Synthesis of 46d

Under nitrogen protection, 17h (180 mg, 0.52 mmol) was dissolved in dry DMF (1.5 mL), and HATU (290 mg, 0.78 mmol) and DIPEA (130 mg, 1.04 mmol) were added. At room temperature, the reaction mixture was stirred for 40 min, and then crude 46c (200 mg, 0.51 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 46d (200 mg, yield: 54.48%).

LC-Ms m/z (ESI): 720.3 [M+H]⁺.

### Step 4: Synthesis of Compounds 46-1 and 46-2

46d (200 mg, 0.28 mmol) was dissolved in methanol (3 mL) and water (1 mL), lithium hydroxide monohydrate (45 mg, 1.08 mmol) was added, and the resulting mixture was reacted at room temperature for 5 h. The reaction mixture was adjusted with 1 N hydrochloric acid to pH 5-6 and concentrated under reduced pressure to afford crude compound 46, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% ammonium acetate)) to afford compound 46-1 (20 mg, retention time=4.111 min, yield: 10.39%) and compound 46-2 (20 mg, retention time=4.197 min, yield: 10.39%).

Compound 46-1:

LC-Ms m/z (ESI): 692.3 [M+H]⁺.

¹H NMR (400 MHz, CD₃OD) δ 7.88 (s, 1H), 7.13-7.04 (m, 2H), 6.98 (t, 1H), 6.83 (s, 1H), 5.82-5.73 (m, 1H), 5.71-5.62 (m, 1H), 3.35-3.24 (m, 2H), 3.15-2.86 (m, 10H), 2.08-1.77 (m, 11H), 1.47-1.35 (m, 1H), 1.00-0.91 (m, 6H).

Compound 46-2:

LC-Ms m/z (ESI): 692.3 [M+H]⁺.

¹H NMR (400 MHz, CD₃OD) δ 7.91 (s, 1H), 7.18-7.09 (m, 2H), 7.01 (t, 1H), 6.89 (s, 1H), 5.81-5.74 (m, 1H), 5.72-5.64 (m, 1H), 3.36-3.26 (m, 2H), 3.11-2.89 (m, 10H), 2.06 (s, 3H), 1.99 (s, 3H), 1.93 (s, 3H), 1.86-1.71 (m, 2H), 1.36-1.25 (m, 1H), 0.95-0.84 (m, 6H).

### Example 47: Preparation of Compound 47 and Stereoisomer Thereof

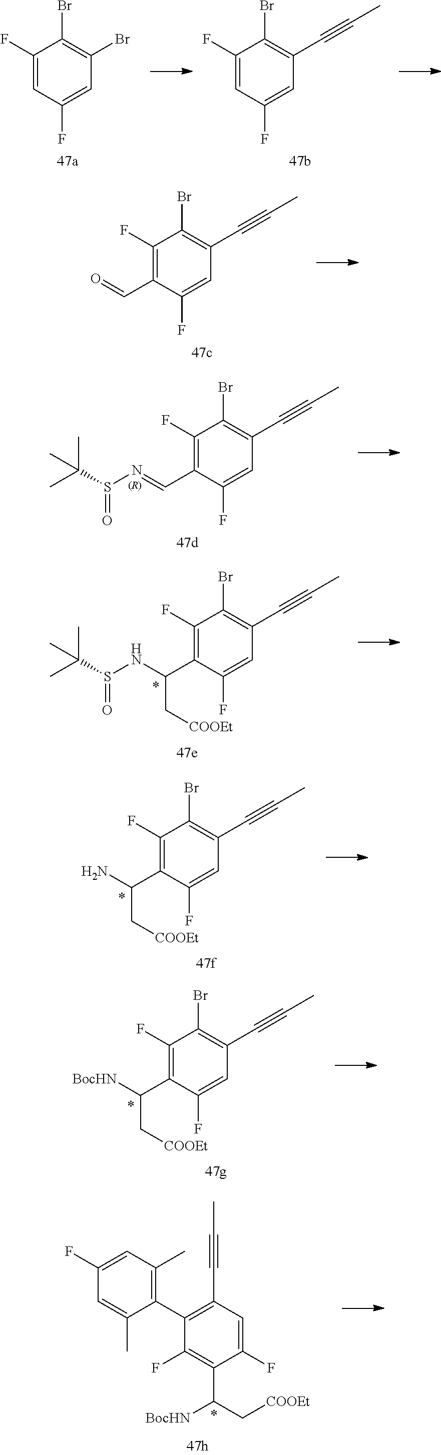

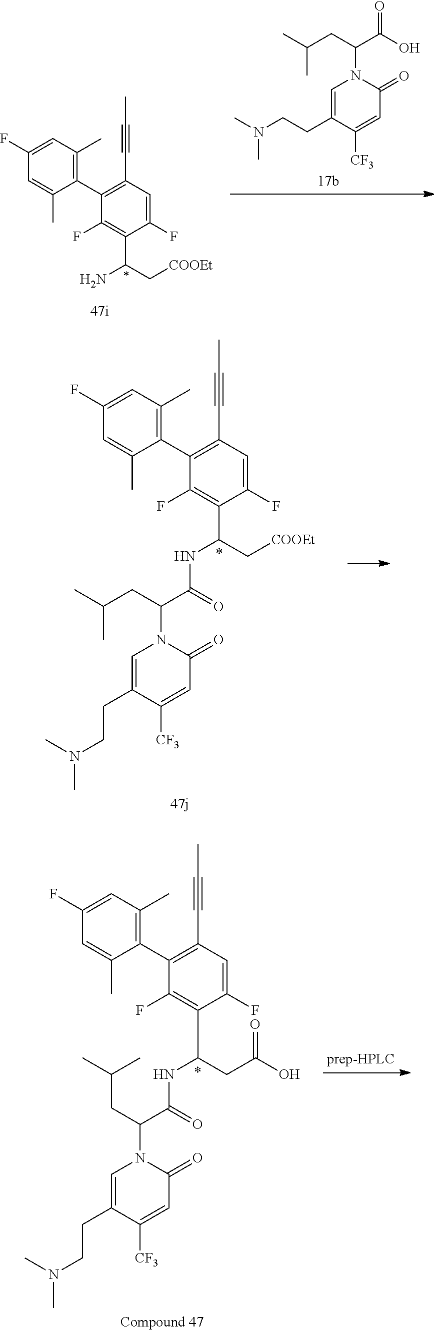 [SMILES: CC#Cc1cc(F)c(C(N)CC(=O)OCC)c(F)c1-c1c(C)cc(F)cc1C ; CC(C)CC(C(=O)O)n1cc(CCN(C)C)c(C(F)(F)F)cc1=O ; CC#Cc1cc(F)c(C(CC(=O)OCC)NC(=O)C(CC(C)C)n2cc(CCN(C)C)c(C(F)(F)F)cc2=O)c(F)c1-c1c(C)cc(F)cc1C ; CC#Cc1cc(F)c(C(CC(=O)O)NC(=O)C(CC(C)C)n2cc(CCN(C)C)c(C(F)(F)F)cc2=O)c(F)c1-c1c(C)cc(F)cc1C]

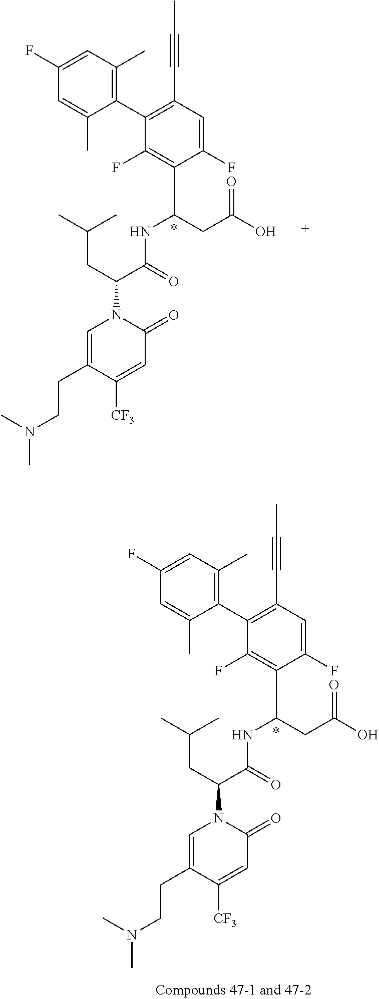 [SMILES: CC#Cc1cc(F)c(C(CC(=O)O)NC(=O)[C@@H](CC(C)C)n2cc(CCN(C)C)c(C(F)(F)F)cc2=O)c(F)c1-c1c(C)cc(F)cc1C ; CC#Cc1cc(F)c(C(CC(=O)O)NC(=O)[C@H](CC(C)C)n2cc(CCN(C)C)c(C(F)(F)F)cc2=O)c(F)c1-c1c(C)cc(F)cc1C]

### Step 1: Synthesis of 47b

Under nitrogen-replete conditions, 47a (5.0 g, 18.66 mmol), cuprous iodide (0.7 g, 3.73 mmol) and PdCl₂ (PPh₃)₂ (1.3 g, 1.87 mmol) were successively added to a sealed tube, followed by DMF (50 mL), and compounds cesium fluoride (8.5 g, 55.98 mmol) and trimethylsilylpropyne (2.5 g, 22.39 mmol), and the resulting mixture was reacted at 65° C. for 48 h. The reaction mixture was cooled to room temperature and water (200 mL) was added. The resulting mixture was extracted with ethyl acetate (200 mL×3) and the organic phases were combined, dried over anhydrous sodium sulfate and filtered. The filtrate was concentrated under reduced pressure and the residue was purified by silica gel column chromatography to afford 47b (4.0 g, yield: 74.23%).

### Step 2: Synthesis of 47c

Under nitrogen atmosphere, 47b (4.0 g, 17.31 mmol) was dissolved in THF (40 mL) and at −78° C., lithium diisopropylamide (14 mL) was added. The mixture was stirred for another 1 h. DMF (4 mL) was slowly added dropwise and the resulting mixture was reacted at −78° C. for 1 h. After the dropwise addition was completed, the reaction was quenched with saturated aqueous ammonium chloride solution (150 mL). The reaction mixture was extracted with ethyl acetate (100×3) and the organic layers were combined, washed with brine, dried over anhydrous sodium sulfate and filtered. The filtrate was concentrated under reduced pressure and the residue was subjected to silica gel column chromatography to afford 47c (4.2 g, yield: 93.66%).

### Step 3: Synthesis of 47d

Under nitrogen protection, compound 47c (1.1 g, 4.25 mmol) and (R)-(+)-tert-butylsulfinamide (0.7 g, 5.10 mmol) were dissolved in tetrahydrofuran (15 mL) and at 0° C., tetrabutyl titanate (1.86 g, 8.16 mmol) was added. The resulting mixture was stirred overnight. The reaction mixture was filtered, the filter cake was washed with ethyl acetate, and the filtrate was extracted with ethyl acetate. The organic phase was washed with water, dried over anhydrous sodium sulfate, filtered and concentrated under reduced pressure. The residue was purified by chromatographic column to afford 47d (1.3 g, yield: 84.44%).

### Step 4: Synthesis of 47

Under nitrogen protection, CuCl (1.1 g, 10.77 mmol) and zinc powder (3.3 g, 50.26 mmol) were dissolved in tetrahydrofuran (20 mL) and the mixture was reacted at 60° C. for 2 h and cooled to room temperature. Ethyl bromoacetate (3.0 g, 17.95 mmol) was added and the resulting mixture was reacted at 60° C. for 1 h and cooled to room temperature. Under ice bath conditions, 47d (1.3 g, 3.59 mmol) was added and the reaction mixture was reacted for 2 h. The reaction system was extracted with ethyl acetate and the organic phase was washed with water, dried over anhydrous sodium sulfate, filtered and concentrated under reduced pressure. The residue was purified by chromatographic column to afford 47e (1.5 g, yield: 92.78%).

### Step 5: Synthesis of 47f

47e (1.2 g, 2.66 mmol) was dissolved in acetonitrile (12 mL), 4N hydrogen chloride-1,4-dioxane solution (6 mL) was added, and the resulting mixture was stirred at room temperature for 30 min. The reaction mixture was concentrated under reduced pressure to afford the hydrochloride of crude 47f (1.2 g).

LC-Ms m/z (ESI): 346.0 [M+H]⁺.

### Step 6: Synthesis of 47g

47f (1.2g, crude) was dissolved in DCM (15 mL), triethylamine (1.05 g, 10.40 mmol) and di-tert-butyl dicarbonate (1.2 g, 5.50 mmol) were successively added, and the resulting mixture was reacted at room temperature for 2 h. The reaction mixture was concentrated under reduced pressure and the residue was purified by chromatographic column to afford 47g (1.4 g, yield: 90.49%).

### Step 7: Synthesis of 47h

Under nitrogen protection, 47g (500.0 mg, 1.12 mmol), (4-fluoro-2,6-dimethoxyphenyl)boronic acid (376.3 mg, 2.24 mmol), Ruphos-Pd-G₃ (187.4 mg, 0.22 mmol) and potassium phosphate (713.2 mg, 3.36 mmol) were successively placed into a reaction flask, DMF (6 mL) and water (0.6 mL) were added, and the mixture was stirred at 70° C. for 4 h. The reaction mixture was cooled to room temperature and concentrated under reduced pressure. Water (80 mL) was added and the resulting mixture was extracted with ethyl acetate (100 mL×3). The organic phases were combined, dried over anhydrous sodium sulfate, filtered and concentrated under reduced pressure and the residue was purified by silica gel chromatographic column to afford 47h (300.0 mg, yield: 52.56%).

LC-Ms m/z (ESI): 390.1 [M+H−tBu]⁺.

### Step 8: Synthesis of 47i

47h (300.0 mg, 0.61 mmol) was dissolved in acetonitrile (3 mL), 4 N hydrogen chloride-1,4-dioxane solution (2 mL) was added, and the resulting mixture was stirred at room temperature for 30 min. The reaction mixture was concentrated under reduced pressure to afford the hydrochloride of crude 47i (300 mg).

LC-Ms m/z (ESI): 390.1 [M+H]⁺.

### Step 9: Synthesis of 47j

Under nitrogen protection, 17h (213.2 mg, 0.61 mmol) and HATU (387.8 mg, 1.02 mmol) were dissolved in DMF (4 mL), triethylamine (263.7 mg, 2.04 mmol) was added, and the resulting mixture was reacted at room temperature for 40 min. Crude compound 47i (200.0 mg, 0.51 mmol) was added and the resulting mixture was reacted at room temperature overnight. Water (80 mL) was added and the resulting mixture was extracted with ethyl acetate (100 mL×3). The organic phases were combined, dried over anhydrous sodium sulfate and filtered and the filtrate was concentrated under reduced pressure and then purified by chromatographic column to afford 47j (130.0 mg, yield: 35.41%).

LC-Ms m/z (ESI): 720.2 [M+H]⁺.

### Step 10: Synthesis of Compounds 47-1 and 47-2

47j (130.0 mg, 0.18 mmol) was dissolved in THF (1 mL), 1 mL aqueous solution of lithium hydroxide (8.6 mg, 0.36 mmol) was slowly added dropwise, and the resulting mixture was reacted at room temperature for 5 h. The resulting mixture was diluted with water (6 mL), adjusted with 1 N hydrochloric acid aqueous solution to pH=5-6 and extracted with ethyl acetate (15 mL×3). The organic phases were combined, dried over anhydrous sodium sulfate, filtered and concentrated to afford crude compound 47, which was subjected to prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid)) to afford the trifluoroacetate of compound 47-1 (10.0 mg, yield: 8.02%) and trifluoroacetate of compound 47-2 (20.0 mg, yield: 16.03%)

Trifluoroacetate of compound 47-1:

LC-Ms m/z=692.9 [M+H]⁺.

¹H NMR (400 MHz, CD₃OD) δ 7.88 (s, 1H), 7.05 (d, 1H), 6.87-6.79 (m, 3H), 5.79-5.70 (m, 1H), 5.69-5.59 (m, 1H), 3.30-3.21 (m, 2H), 3.12-2.85 (m, 10H), 2.07-1.84 (m, 8H), 1.76 (s, 3H), 1.47-1.36 (m, 1H), 1.01-0.90 (m, 6H).

Trifluoroacetate of compound 47-1:

LC-Ms m/z=692.9 [M+H]⁺.

¹H NMR (400 MHz, CD₃OD) δ 7.91 (s, 1H), 7.10 (d, 1H), 6.91-6.82 (m, 3H), 5.80-5.72 (m, 1H), 5.70-5.59 (m, 1H), 3.37-3.29 (m, 1H), 3.09-2.82 (m, 11H), 2.00 (s, 6H), 1.87-1.71 (m, 5H), 1.38-1.26 (m, 2H), 0.94-0.83 (m, 6H).

### Example 48: Preparation of Compound 48 and Stereoisomer Thereof

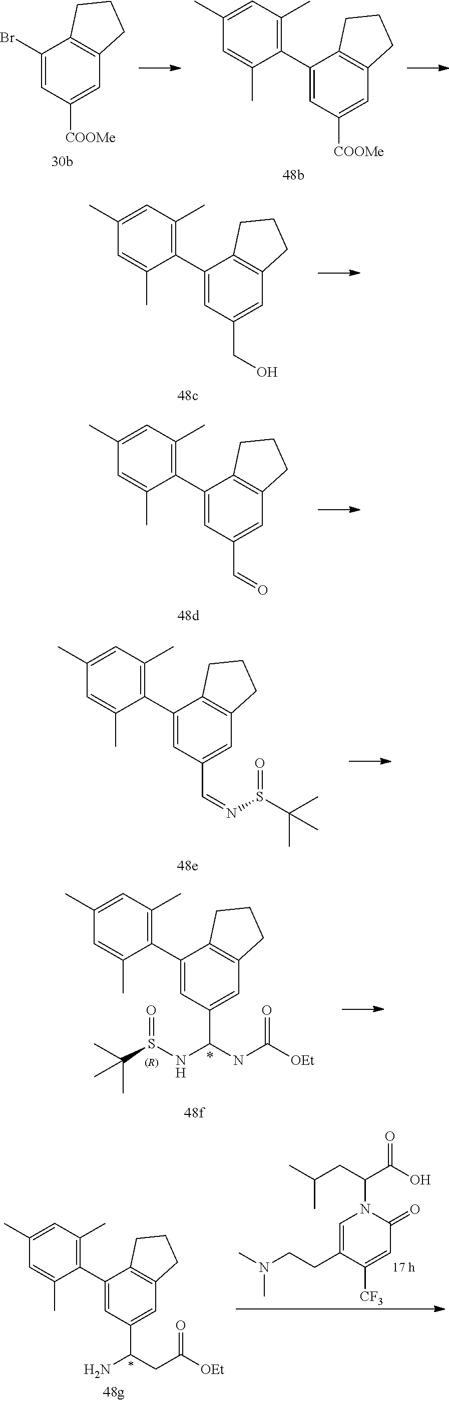

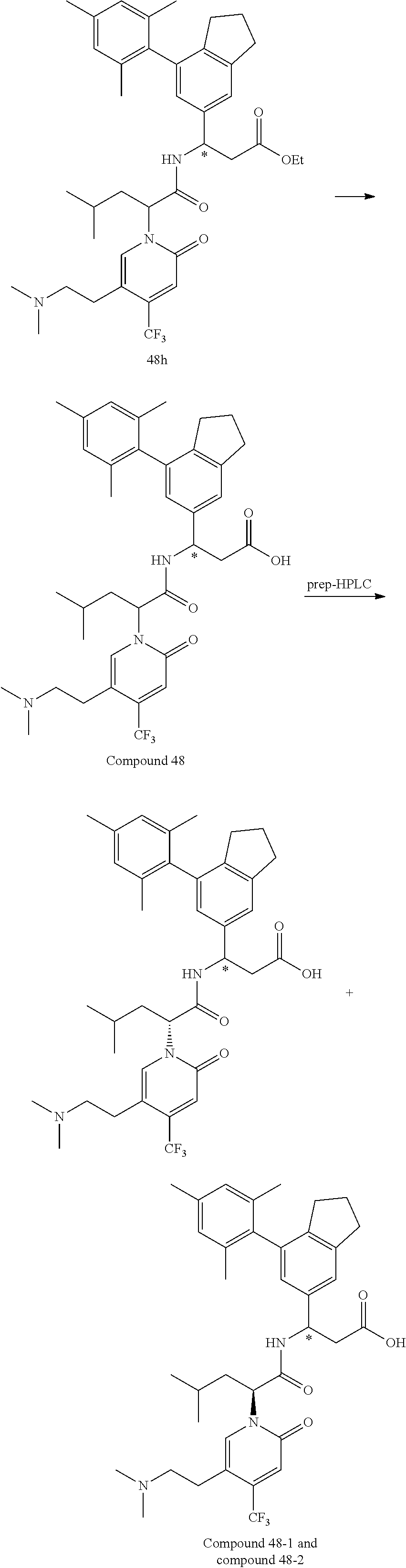

### Step 1: Synthesis of 48b

Under nitrogen protection, 30b (3.2 g, 12.45 mmol), 2,4,6-trimethylphenylboronic acid (2.45 g, 14.94 mmol) and cesium carbonate (7.66 g, 23.52 mmol) were added to 1,4-dioxane (30.0 mL) and water (3.0 mL), followed by Ruphos-Pd-G3 (1.4 g, 1.25 mmol) and potassium phosphate (7.9 g, 37.35 mmol), and the resulting mixture was reacted at 80° C. in a sealed tube for 5 h. The reaction system was cooled to room temperature and filtered through celite. Ethyl acetate (50 mL) was added and the mixture was successively washed with water (30 mL) and saturated brine (30 mL), dried over anhydrous sodium sulfate, filtered and concentrated under reduced pressure. The residue was subjected to flash chromatography on a silica gel column to afford 48b (2.9 g, yield: 78.59%).

### Step 2: Synthesis of 48c

Under nitrogen protection, 48b (2.9 g, 9.85 mmol) was dissolved in dry tetrahydrofuran (30 mL) and at 0° C., lithium aluminum hydride (560.0 mg, 14.77 mmol) was slowly added. The resulting mixture was reacted at room temperature for 1 h. 10% sodium sulfate aqueous solution (20 mL) was added and the mixture was filtered and concentrated under reduced pressure. The residue was subjected to flash chromatography on a silica gel column to afford 48c (1.2 g, yield: 45.76%).

### Step 3: Synthesis of Compound 48d

Under nitrogen protection, 48c (1.2 g, 4.50 mmol) was dissolved in dry dichloromethane (10 mL), sodium bicarbonate (0.76 g, 9.04 mmol) and Dess-Martin periodinane (2.86 g, 6.75 mmol) were added at room temperature, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate and filtered. The solvent was removed under reduced pressure and the residue was subjected to column chromatography on a silica gel column to afford 48d (0.66 g, 55.48%).

### Step 4: Synthesis of 48e

Under nitrogen protection, 48d (600.0 mg, 2.27 mmol) and R-tert-butylsulfinamide (373.7 mg, 2.72 mmol) were dissolved in tetrahydrofuran (6 mL), tetraethyl titanate (2776.7 mg, 3.41 mmol) was slowly added, and the resulting mixture was reacted at 45° C. for 15 h and concentrated under reduced pressure to afford a crude, which was subjected to flash column chromatography on a silica gel column to afford 48e (500.0 mg, yield: 59.93%).

### Step 5: Synthesis of 48f

Zinc powder (1.3 g, 19.04 mmol) was added to dry tetrahydrofuran (5 mL), the mixture was subjected to nitrogen replacement three times, CuCl (403.9 mg, 4.08 mmol) was added, and the resulting mixture was reacted at 60° C. for 2 h. The reaction mixture was cooled to room temperature, ethyl bromoacetate (1.2 g, 6.80 mmol) was slowly added, and the resulting mixture was reacted at 60° C. for 1 h and cooled to 0° C. A solution of 48e (500.0 mg, 1.36 mmol) in tetrahydrofuran (1 mL) was added and the mixture was stirred at 0° C. for 3 h. The reaction mixture was filtered through celite and saturated ammonium chloride solution (20 mL) was added. The resulting mixture was extracted with ethyl acetate (30 ml×3), washed with saturated brine (20 mL), dried over anhydrous sodium sulfate, filtered and concentrated under reduced pressure. The residue was subjected to flash column chromatography to afford 48f (500.0 mg, yield: 80.69%).

LC-Ms m/z (ESI): 456.4 [M+H]⁺.

### Step 6: Synthesis of 48g

48f (496.6 mg, 1.09 mmol) was dissolved in tetrahydrofuran (5 mL), 4 N hydrogen chloride dioxane solution (5 mL) was added, and the resulting mixture was stirred at room temperature for 3 h. The reaction mixture was concentrated under reduced pressure to afford crude 48g.

LC-Ms m/z (ESI): 352.3 [M+H]⁺.

### Step 7: Synthesis of 48h

Under nitrogen protection, 17h (348.4 mg, 1.00 mmol) was dissolved in dry DMF (1.5 mL), and HOBt (383.4 mg, 2.00 mmol), EDCI (348.4 mg, 2.00 mmol) and DIPEA (517.0 mg, 4.00 mmol) were successively added. At room temperature, the reaction mixture was stirred for 40 min, and then crude 48g (350.0 mg, 1.00 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 48h (145 mg, yield: 72.91%).

LC-Ms m/z (ESI): 682.7 [M+H]⁺.

### Step 8: Synthesis of Compounds 48-1 and 48-2

48h (290.0 mg, 0.43 mmol) was dissolved in tetrahydrofuran (3 mL) and water (1 mL), lithium hydroxide monohydrate (20.6 mg, 0.86 mmol) was added, and the resulting mixture was reacted at room temperature for 5 h. The reaction mixture was adjusted with 1 N hydrochloric acid to pH 5-6 and concentrated under reduced pressure to afford crude compound 48, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid) to afford the trifluoroacetate of compound 48-1 (41 mg, retention time=4.252 min, yield: 14.58%) and trifluoroacetate of compound 48-2 (40 mg, retention time=4.254 min, yield: 14.23%).

Trifluoroacetate of compound 48-1:

LC-Ms m/z (ESI): 654.8 [M+H]⁺.

¹H NMR (400 MHz, DMSO-d₆) δ 12.22 (br.s, 1H), 8.96 (d, 1H), 7.94 (s, 1H), 7.10 (s, 1H), 6.92-6.85 (m, 2H), 6.77-6.69 (m, 2H), 5.67-5.57 (m, 1H), 5.21-5.10 (m, 1H), 3.21-3.05 (m, 2H), 2.93-2.76 (m, 10H), 2.74-2.68 (m, 2H), 2.38-2.30 (m, 2H), 2.25 (s, 3H), 2.00-1.88 (m, 3H), 1.85-1.72 (m, 7H), 1.39-1.26 (m, 1H), 0.94-0.81 (m, 6H).

Trifluoroacetate of compound 48-1:

LC-Ms m/z (ESI): 654.8 [M+H]⁺.

¹H NMR (400 MHz, DMSO-d₆) δ 12.22 (br.s, 1H), 9.07 (d, 1H), 7.96 (s, 1H), 7.12 (s, 1H), 6.92 (s, 2H), 6.81 (s, 1H), 6.77 (s, 1H), 5.68-5.59 (m, 1H), 5.19-5.09 (m, 1H), 3.22-3.07 (m, 2H), 2.95-2.78 (m, 10H), 2.76-2.62 (m, 2H), 2.41-2.33 (m, 2H), 2.27 (s, 3H), 2.02-1.90 (m, 2H), 1.88-1.74 (m, 7H), 1.67-1.56 (m, 1H), 1.27-1.13 (m, 1H), 0.83-0.72 (m, 6H).

### Example 49: Preparation of Compound 49 and Stereoisomer Thereof

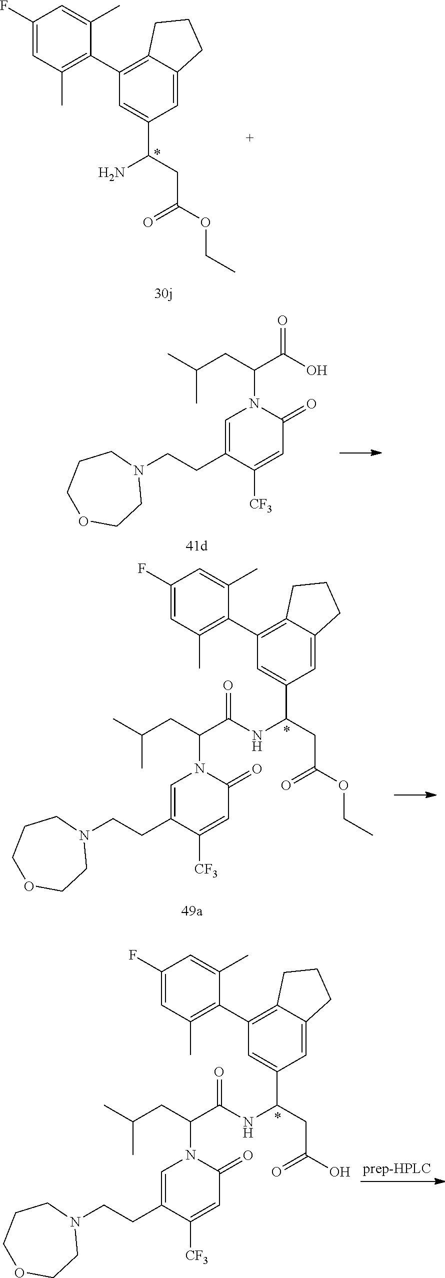 [SMILES: CCOC(=O)CC(N)c1cc2c(c(-c3c(C)cc(F)cc3C)c1)CCC2 ; CC(C)CC(C(=O)O)n1cc(CCN2CCCOCC2)c(C(F)(F)F)cc1=O ; CCOC(=O)CC(NC(=O)C(CC(C)C)n1cc(CCN2CCCOCC2)c(C(F)(F)F)cc1=O)c1cc2c(c(-c3c(C)cc(F)cc3C)c1)CCC2 ; Cc1cc(F)cc(C)c1-c1cc(C(CC(=O)O)NC(=O)C(CC(C)C)n2cc(CCN3CCCOCC3)c(C(F)(F)F)cc2=O)cc2c1CCC2]

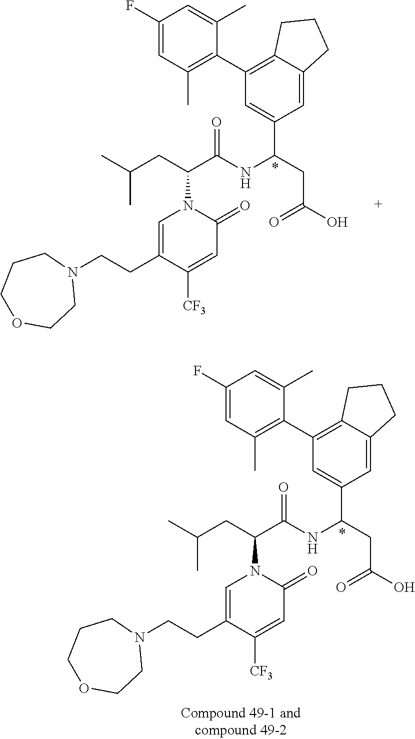 [SMILES: Cc1cc(F)cc(C)c1-c1cc(C(CC(=O)O)NC(=O)[C@@H](CC(C)C)n2cc(CCN3CCCOCC3)c(C(F)(F)F)cc2=O)cc2c1CCC2 ; Cc1cc(F)cc(C)c1-c1cc(C(CC(=O)O)NC(=O)[C@H](CC(C)C)n2cc(CCN3CCCOCC3)c(C(F)(F)F)cc2=O)cc2c1CCC2]

### Step 1: Synthesis of 49a

Under nitrogen protection, 41d (304 mg, 0.75 mmol) and 30j (270 mg, 0.75 mmol) were dissolved in dry DMF (6 mL), and EDCI (290 mg, 1.50 mmol), HOBT (200 mg, 1.50 mmol) and DIPEA (0.37 mL, 2.25 mmol) were added. The reaction mixture was stirred and reacted at room temperature for 2 h, diluted with water, and extracted with ethyl acetate. The organic phases were combined, dried over anhydrous sodium sulfate, filtered and concentrated under reduced pressure. The residue was subjected to column chromatography on a silica gel column to afford 49a (284 mg, yield: 51.04%).

LC-Ms m/z (ESI): 742.9 [M+H]⁺.

### Step 2: Synthesis of Compounds 49-1 and 49-2

49a (284 mg, 0.38 mmol) was dissolved in tetrahydrofuran (3 mL) and water (2 mL), lithium hydroxide monohydrate (32 mg, 0.76 mmol) was added, and the resulting mixture was reacted at room temperature for 3 h. The reaction mixture was concentrated under reduced pressure to afford crude compound 49, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid) to afford the trifluoroacetate of compound 49-1 (40 mg, retention time=5.420 min, yield: 14.75%) and trifluoroacetate of compound 49-2 (40 mg, retention time=5.491 min, yield: 14.75%).

Trifluoroacetate of compound 49-1:

LC-Ms m/z (ESI): 714.4 [M+H]⁺.

¹H NMR (400 MHz, DMSO-d₆) δ 9.58 (s, 1H), 8.99 (d, 1H), 7.96 (s, 1H), 7.13 (s, 1H), 6.97-6.91 (m, 2H), 6.77-6.72 (m, 2H), 5.65-5.58 (m, 1H), 5.19-5.12 (m, 1H), 3.93-3.53 (m, 6H), 3.28-3.15 (m, 2H), 2.93-2.83 (m, 3H), 2.74-2.68 (m, 2H), 2.38-2.30 (m, 2H), 2.12-2.02 (m, 2H), 2.00-1.91 (m, 3H), 1.86 (s, 3H), 1.79 (s, 3H), 1.40-1.28 (m, 1H), 0.89 (d, 3H), 0.85 (d, 3H).

Trifluoroacetate of compound 49-2:

LC-Ms m/z (ESI): 714.4 [M+H]⁺.

¹H NMR (400 MHz, DMSO-d₆) δ 9.56 (s, 1H), 9.09 (d, 1H), 7.97 (s, 1H), 7.15 (s, 1H), 6.99 (s, 1H), 6.97 (s, 1H), 6.82 (s, 1H), 6.78 (s, 1H), 5.67-5.61 (m, 1H), 5.20-5.10 (m, 1H), 3.93-3.61 (m, 6H), 3.30-3.18 (m, 3H), 2.95-2.85 (m, 3H), 2.75-2.64 (m, 2H), 2.41-2.30 (m, 2H), 2.10-1.94 (m, 5H), 1.90 (s, 6H), 1.84-1.75 (m, 1H), 1.68-1.58 (m, 1H), 1.21-1.11 (m, 1H), 0.82-0.73 (m, 6H).

### Example 50: Preparation of Compound 50 and Stereoisomer Thereof

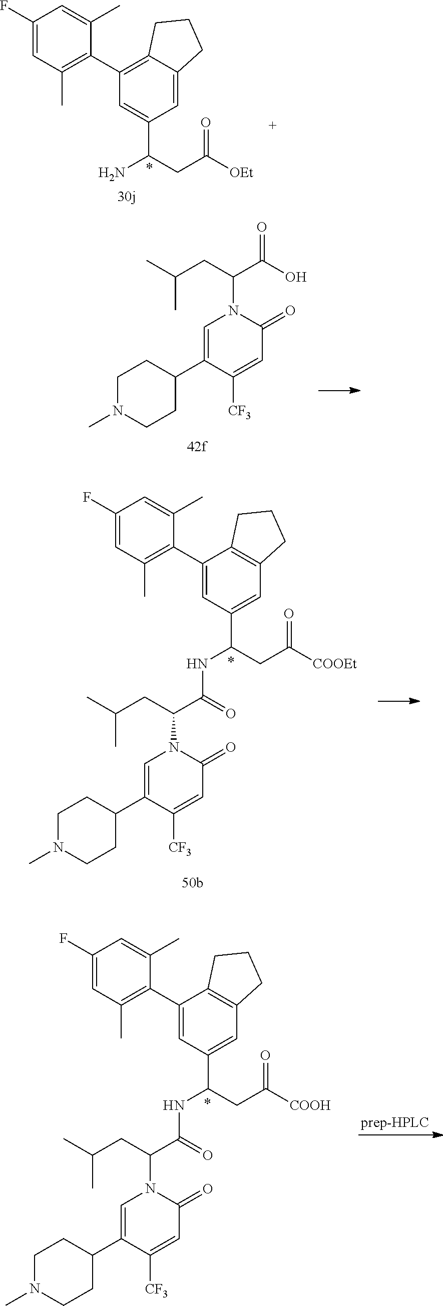 [SMILES: CCOC(=O)CC(N)c1cc2c(c(-c3c(C)cc(F)cc3C)c1)CCC2 ; CC(C)CC(C(=O)O)n1cc(C2CCN(C)CC2)c(C(F)(F)F)cc1=O ; CCOC(=O)C(=O)CC(NC(=O)[C@@H](CC(C)C)n1cc(C2CCN(C)CC2)c(C(F)(F)F)cc1=O)c1cc2c(c(-c3c(C)cc(F)cc3C)c1)CCC2 ; Cc1cc(F)cc(C)c1-c1cc(C(CC(=O)C(=O)O)NC(=O)C(CC(C)C)n2cc(C3CCN(C)CC3)c(C(F)(F)F)cc2=O)cc2c1CCC2]

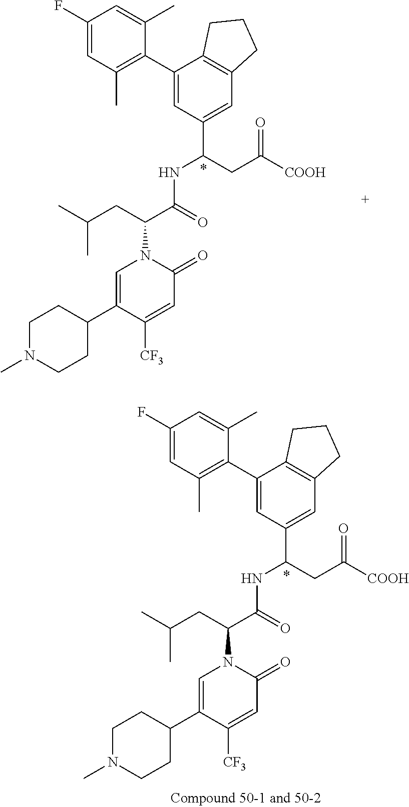 [SMILES: Cc1cc(F)cc(C)c1-c1cc(C(CC(=O)C(=O)O)NC(=O)[C@@H](CC(C)C)n2cc(C3CCN(C)CC3)c(C(F)(F)F)cc2=O)cc2c1CCC2 ; Cc1cc(F)cc(C)c1-c1cc(C(CC(=O)C(=O)O)NC(=O)[C@H](CC(C)C)n2cc(C3CCN(C)CC3)c(C(F)(F)F)cc2=O)cc2c1CCC2]

### Step 1: Synthesis of 50b

Under nitrogen protection, 42f (200 mg, 0.53 mmol) was dissolved in dry DMF (6 mL), and EDCI (200 mg, 1.06 mmol), HOBT (140 mg, 1.06 mmol) and DIPEA (270 mg, 2.12 mmol) were added. At room temperature, the reaction mixture was stirred for 40 min, and then crude 30j (190 mg, 0.53 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 50b (320 mg, yield: 84.82%).

LC-Ms m/z (ESI): 712.8 [M+H]⁺.

### Step 2: Synthesis of Compounds 50-1 and 50-2

50b (320 mg, 0.45 mmol) was dissolved in tetrahydrofuran (12 mL) and water (4 mL), 1 5 7-triazidobicyclo(4.4.0)dec-5-ene (190 mg, 1.36 mmol) was added, and the resulting mixture was reacted at room temperature for 5 h. The reaction mixture was adjusted with 1 N hydrochloric acid to pH 5-6 and concentrated under reduced pressure to afford crude compound 50, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid) to afford the trifluoroacetate of compound 50-1 (16 mg, retention time=4.226 min, yield: 5.20%) and trifluoroacetate of compound 50-2 (15 mg, retention time=4.271 min, yield: 4.87%).

Trifluoroacetate of compound 50-1:

LC-Ms m/z (ESI): 684.7 [M+H]⁺.

¹H NMR (400 MHz, CD₃OD) δ 7.79 (s, 1H), 7.16 (s, 1H), 6.84-6.68 (m, 4H), 5.78-5.71 (m, 1H), 5.37-5.27 (m, 1H), 3.64-3.51 (m, 2H), 3.22-3.08 (m, 2H), 2.98-2.80 (m, 8H), 2.45-2.35 (m, 2H), 2.18-2.08 (m, 1H), 2.03-1.76 (m, 13H), 1.52-1.38 (m, 1H), 1.02-0.91 (m, 6H).

Trifluoroacetate of compound 50-2:

LC-Ms m/z (ESI): 684.7[M+H]⁺.

¹H NMR (400 MHz, CD₃OD) δ 7.85 (s, 1H), 7.23 (s, 1H), 6.90-6.78 (m, 4H), 5.77-5.69 (m, 1H), 5.35-5.24 (m, 1H), 3.66-3.55 (m, 2H), 3.23-3.11 (m, 2H), 3.02-2.75 (m, 8H), 2.51-2.40 (m, 2H), 2.19-2.10 (m, 2H), 2.08-1.67 (m, 12H), 1.38-1.25 (m, 1H), 0.93-0.81 (m, 6H).

### Example 51: Preparation of Compound 51 and Stereoisomer Thereof

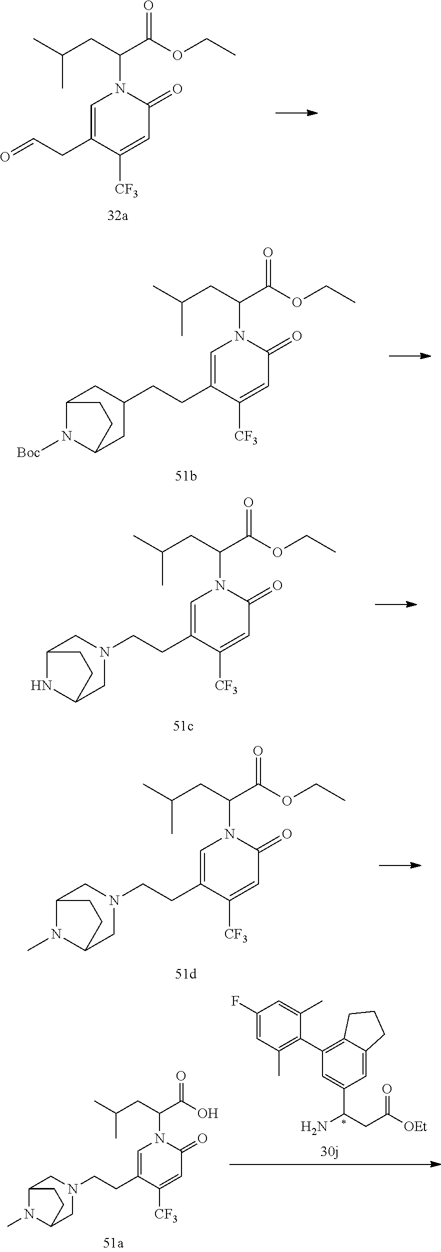

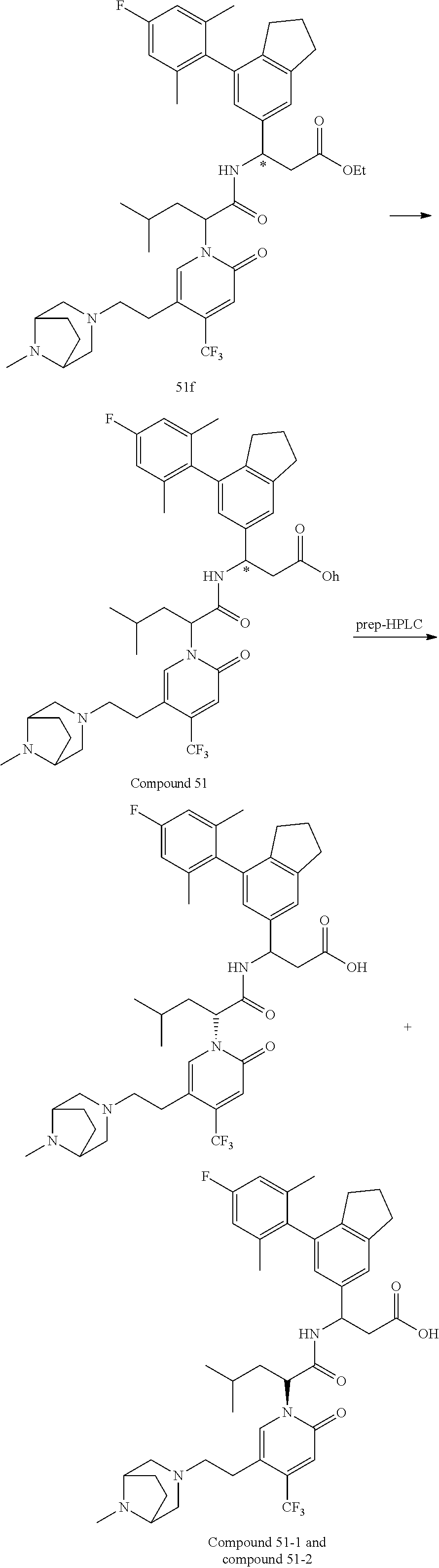

### Step 1: Synthesis of 51b

32a (5 g, 14.4 mmol) was added to ultra-dry DCE (100 mL), tert-butyl 3,8-diazabicyclo[3.2.1]octane-8-carboxylate (3.06 g, 14.4 mmol), AcOH (0.086 g, 1.44 mmol) and 4 Å molecular sieve (100 mg) were successively added, and the mixture was reacted at room temperature for 1 h. Sodium triacetoxyborohydride (9.16 g, 43.2 mmol) was added and the mixture was reacted for 1 h. Ethyl acetate (80 mL) and saturated aqueous NaHCO₃ solution (50 mL) were added for extraction and separation. The aqueous phase was extracted with ethyl acetate (40 mL×1) and the organic phases were combined, washed with saturated brine (40 mL×1), dried over anhydrous sodium sulfate and concentrated under reduced pressure. The residue was subjected to column chromatography on a silica gel chromatographic column to afford 51b (4.8 g, 61.3%).

LC-Ms m/z (ESI): 544.6[M+H]⁺.

### Step 2: Synthesis of 51c

51b (2 g, 3.68 mmol) was dissolved in dichloromethane (10 mL), 4 N hydrogen chloride dioxane solution (15 mL) was added, and the resulting mixture was stirred at room temperature for 3 h. The reaction mixture was concentrated under reduced pressure to afford crude 51c.

LC-Ms m/z (ESI): 444.6 [M+H]⁺.

### Step 3: Synthesis of 51d

51c (1.6 g, 3.61 mmol) was added to ultra-dry DCE (50 mL), paraformaldehyde (480 mg, 16.26 mmol), AcOH (0.11 g, 1.80 mmol) and 4 Å molecular sieve (100 mg) were successively added, and the mixture was reacted at room temperature for 1 h. Sodium triacetoxyborohydride (1.91 g, 9.03 mmol) was added and the mixture was reacted for 1 h. Ethyl acetate (80 mL) and saturated aqueous NaHCO₃ solution (50 mL) were added for extraction and separation. The aqueous phase was extracted with ethyl acetate (40 mL×1) and the organic phases were combined, washed with saturated brine (40 mL×1), dried over anhydrous sodium sulfate and concentrated under reduced pressure. The residue was subjected to column chromatography on a silica gel chromatographic column to afford 51d (1.4 g, 84.76%).

LC-Ms m/z (ESI): 458.5[M+H]⁺.

### Step 4: Synthesis of 51e

51d (1.4 g, 3.06 mmol) was dissolved in tetrahydrofuran (6 mL) and water (2 mL), lithium hydroxide monohydrate (220 mg, 9.18 mmol) was added, the resulting mixture was reacted at room temperature for 5 h. The reaction mixture was adjusted with 1 N hydrochloric acid to pH 5-6, concentrated under reduced pressure and then purified by reverse phase column to afford 51e (1.2 g, 91.31%).

LC-Ms m/z (ESI): 430.4[M+H]⁺.

### Step 5: Synthesis of 51f

Under nitrogen protection, 51e (330 mg, 0.76 mmol) was dissolved in dry DMF (6 mL), and EDCI (290 mg, 1.51 mmol), HOBT (210 mg, 1.55 mmol) and DIPEA (300 mg, 2.30 mmol) were added. At room temperature, the reaction mixture was stirred for 40 min, and then crude 30j (270 mg, 0.76 mmol) was added. The resulting mixture was reacted at room temperature overnight. Ethyl acetate (80 mL) was added and the resulting mixture was successively washed with water (20 mL×2) and saturated brine (20 mL×1), dried over anhydrous sodium sulfate and filtered and the filtrate was concentrated under reduced pressure and subjected to column chromatography on a silica gel column to afford 51f (399 mg, yield: 68.5%).

LC-Ms m/z (ESI): 767.9[M+H]⁺.

### Step 6: Synthesis of Compounds 51-1 and 51-2

51f (399 mg, 0.36 mmol) was dissolved in tetrahydrofuran (6 mL) and water (2 mL), 1 5 7-triazidobicyclo(4.4.0)dec-5-ene (220 mg, 1.58 mmol) was added, and the resulting mixture was reacted at room temperature for 5 h. The reaction mixture was adjusted with 1 N hydrochloric acid to pH 5-6 and concentrated under reduced pressure to afford crude compound 51, which was separated and purified by prep-HPLC (instrument: waters 2767 preparative chromatographic column: SunFire@Prep C18 (19 mm×150 mm); composition of mobile phases: mobile phase A: acetonitrile, and mobile phase B: water (containing 0.1% trifluoroacetic acid) to afford the trifluoroacetate of compound 51-1 (16 mg, retention time=4.273 min, yield: 4.16%) and trifluoroacetate of compound 51-2 (15 mg, retention time=4.342 min, yield: 3.90%).

Trifluoroacetate of compound 51-1:

LC-Ms m/z (ESI): 739.8[M+H]⁺.

¹H NMR (400 MHz, CD₃OD) δ 9.02 (d, 1H), 7.86 (s, 1H), 7.15 (s, 1H), 6.85-6.69 (m, 4H), 5.75-5.67 (m, 1H), 5.40-5.28 (m, 1H), 3.90-3.82 (m, 2H), 3.04-2.88 (m, 4H), 2.86-2.82 (m, 2H), 2.81-2.65 (m, 5H), 2.65-2.49 (m, 4H), 2.44-2.37 (m, 2H), 2.22-2.05 (m, 4H), 2.04-1.93 (m, 3H), 1.90-1.81 (m, 4H), 1.79 (s, 3H), 1.53-1.43 (m, 1H), 1.01-0.92 (m, 6H).

Trifluoroacetate of compound 51-2:

LC-Ms m/z (ESI): 739.8[M+H]⁺.

¹H NMR (400 MHz, CD₃OD) δ 9.04 (d, 1H), 7.92 (s, 1H), 7.23 (s, 1H), 6.88-6.79 (m, 4H), 5.76-5.66 (m, 1H), 5.36-5.26 (m, 1H), 3.91-3.83 (m, 2H), 3.06-2.94 (m, 4H), 2.89-2.78 (m, 5H), 2.77-2.66 (m, 4H), 2.67-2.50 (m, 2H), 2.49-2.41 (m, 2H), 2.25-1.98 (m, 6H), 1.97-1.91 (m, 6H), 1.90-1.81 (m, 1H), 1.80-1.68 (m, 1H), 1.42-1.27 (m, 1H), 0.92-0.81 (m, 6H).

Control compound A has the following structure (see WO 2021076890A1 for the preparation method):

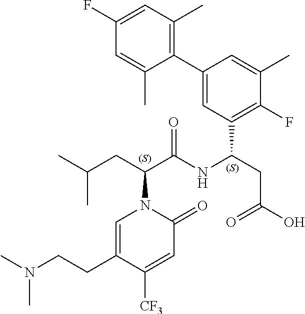

### Biological Test:

### 1. ELISA Detection Assay for Integrin α4β7:

MAdCAM was formulated with TBS buffer to a final concentration of 2.5 ug/ml, 50 ul of the solution was transferred to a 96-well plate, and the plate was coated with same at 4° C. overnight. The plate was washed with TBS buffer three times, then 150 ul of a blocking solution (TBS buffer containing 1% BSA) was added, and the plate was blocked at 37° C. for 1 h. The plate was washed with TBS buffer three times. 1 ug/ml α4β7 integrin was formulated using TBS buffer containing 0.1% BSA. 50 ul of integrin was transferred to a 96-well plate, then 1 ul of the compound at different concentrations or DMSO was added, and the plate was incubated at normal temperature for 2 h. 1 ug/ml biotinylated anti-β7 antibody was formulated using TBS buffer containing 0.1% BSA. The plate was washed with TBS buffer three times, 50 ul of the antibody was added, and the plate was incubated at normal temperature for 1 h. The plate was washed with TBS buffer three times, 50 ul of streptavidin-HRP was added, and the plate was incubated at normal temperature for 20 min. The plate was washed with TBS buffer three times, 50 ul of a TMB substrate was added, and the plate was incubated at normal temperature for 5-30 min. Finally, 25 ul of a termination buffer (phosphate solution at a high concentration) was added. The OD value of the plate was read at 450 nm with a microplate reader. The IC₅₀ value was calculated using GraphPad Prism 6 software.

Conclusion: the compound of the present disclosure has good inhibitory activity on integrin α4β7.

### 2. Integrin α4β7-Mediated Cell Adhesion Assay

MAdCAM-1 was formulated with TBS buffer to a final concentration of 2 μg/ml, 50 μl of the solution was transferred to a 96-well plate, and the plate was coated with same at 4° C. overnight. The plate was washed with TBS buffer three times, then 150 μl of a blocking solution (TBS buffer containing 1% BSA) was added, and the plate was blocked at 37° C. for 1 h. RPM18866 cells were collected, washed twice with DPBS buffer, and then resuspended to 4×10⁵ cells/ml with TBS buffer. 50 μl of the cell suspension was transferred to a 96-well plate so that the cell density reached 2×10⁵ cells/well. Then 1 μl of the compound at different concentrations or DMSO was added and the plate was incubated at 37° C. for 1 h or 2 h. The plate was washed with TBS buffer to remove non-adherent cells. 50 μl of a substrate (4-nitrophenyl-N-acetyl-β-D-glucosaminide) was added and the plate was incubated at 37° C. for 2 h. Finally, 90 μl of a termination solution (50 mM glycine and 5 mM EDTA, pH 10.4) was added. The OD value of the plate was read at 405 nm with a microplate reader. The IC₅₀ value was calculated using GraphPad Prism 6 software. The results are shown in Table 1.

### 3. Integrin α4β1-Mediated Cell Adhesion Assay

VCAM was formulated into a final concentration of 0.5 μg/ml with a coating buffer, 50 μl of the solution was transferred to a 96-well plate, and the plate was coated with same at 4° C. overnight. The plate was washed with coating buffer three times, then 150 μl of a blocking solution (coating buffer containing 1% BSA) was added, and the plate was blocked at 37° C. for 1 h. Jurkat cells were collected, washed twice using DPBS buffer, and then resuspended to 4×106 cells/ml with an action buffer. 50 μl of the cell suspension was transferred to a 96-well plate so that the cell density reached 2×105 cells/well. Then 1 μl of the test compound at different concentration or DMSO was added and the plate was incubated at 37° C. for 1 h. The plate was washed with coating buffer to remove non-adherent cells. 50 μl of a substrate (4-nitrophenyl-N-acetyl-β-D-glucosaminide) was added and the plate was incubated at 37° C. for 2 h. Finally, 90 μl of a termination solution (50 mM glycine and 5 mM EDTA, pH 10.4) was added. The OD value of the plate was read at 405 nm. The 1_{C} value was calculated using GraphPad Prism 6 software. The results are shown in Table 1.
Coating buffer: DMEM+20 mM HEPES+0.4 mM MnC₂
Action buffer: DMEM+20 mM HEPES+0.1% BSA+0.4 mM MnCl₂
DMEM: dulbecco's modified eagle medium;
HEPES: 2-[4-(2-hydroxyethyl)piperazin-1-yl]ethanesulfonic acid

TABLE 1
Results of effects of compounds on α4β7- and α4β1-mediated cell adhesion

| | RPMI-8866 | Jurkat | |
|---|---|---|---|
| | (α4β7)/MadCAM-1 IC₅₀ | (α4β1)/VCAM-1 IC₅₀ | |
| Compound No. | (nM) | (nM) | α4β1/α4β7 |
| Compound 3-2 | <0.5 | / | / |
| Compound 4-2 | <10 | / | / |
| Compound 5-2 | <10 | / | / |
| Compound 7-2 | <10 | / | / |
| Compound 9 | <10 | / | / |
| Ammonium salt of compound | <10 | / | / |
| 12-4 | | | |
| Compound 13 | <10 | / | / |
| Compound 14 | <0.5 | 126.5 | >500 |
| Compound 15-1 | <10 | / | / |
| Compound 16-2 | <0.5 | 34.73 | >100 |
| Ammonium salt of compound | <0.5 | 6.58 | >100 |
| 17-2 | | | |
| Trifluoroacetate of compound | <0.5 | 130.10 | >500 |
| 18-2 | | | |
| Compound 19-2 | <0.5 | / | / |
| Trifluoroacetate of compound | <0.5 | 36.55 | >100 |
| 20-2 | | | |
| Compound 21-2 | <0.5 | / | / |
| Trifluoroacetate of compound | <10 | / | / |
| 22-2 | | | |
| Trifluoroacetate of compound | <10 | / | / |
| 23-2 | | | |
| Trifluoroacetate of compound | <0.5 | / | / |
| 24-2 | | | |
| Trifluoroacetate of compound | <0.5 | 65.97 | >100 |
| 25-2 | | | |
| Compound 26-2 | <0.5 | / | / |
| Compound 27-2 | <0.5 | 16.34 | >100 |
| Compound 28-2 | <0.5 | / | / |
| Compound 30-2 | <0.5 | 91.06 | 1011.7 |
| Compound 30-1:compound 30-2 | <0.5 | 66.81 | >100 |
| (1:1) | | | |
| Compound 31-2 | <10 | / | / |
| Compound 32-2 | <0.5 | 35.47 | >100 |
| Compound 33-2 | <0.5 | 25.57 | >100 |
| Compound 34-2 | <0.5 | / | / |
| Compound 34-1:compound 34-2 | <1 | / | / |
| (1:1) | | | |
| Compound 36-2 | <10 | / | / |
| Trifluoroacetate of compound | <10 | / | / |
| 37-2 | | | |
| Compound 38-2 | <0.5 | / | / |
| Compound 40-2 | <0.5 | 54.72 | >100 |
| Compound 40-1:compound 40-2 | <10 | 296.9 | >100 |
| (1:1) | | | |
| Compound 42-2 | <0.5 | 3.86 | >100 |
| Compound 43-2 | <0.5 | 34.32 | >90 |
| Compound 43-1 | <20 | >4000 | N/A |
| Compound 43-1:compound 43-2 | <10 | 813.4 | >500 |
| (1:1) | | | |
| Compound 44-2 | <0.5 | 95.23 | >500 |
| Compound 44-1:compound 44-2 | <1 | 68.69 | >100 |
| (1:1) | | | |
| Compound 45-2 | <0.5 | 167.1 | >500 |
| Compound 45-1:compound 45-2 | <2 | 233.1 | >100 |
| (1:1) | | | |
| Trifluoroacetate of compound | <0.5 | 7.93 | >100 |
| 47-2 | | | |
| Trifluoroacetate of compound | <0.5 | 171.7 | >100 |
| 48-2 | | | |
| Trifluoroacetate of compound | <10 | 580.1 | >100 |
| 48-1:trifluoroacetate of | | | |
| compound 48-2 (1:1) | | | |
| Trifluoroacetate of compound | <0.5 | 128.4 | >500 |
| 49-2 | | | |
| Trifluoroacetate of compound | <0.5 | 99.78 | >100 |
| 50-2 | | | |
| Trifluoroacetate of compound | <0.5 | 41.03 | >100 |
| 51-2 | | | |
| Control compound A | 0.062 | 3.14 | 50.6 |

Conclusion: the compounds of the present disclosure have good inhibitory effects on integrin α4β7-mediated cell adhesion. Compared with the control compound A, the compounds of the present disclosure have better selectivity for α4β1/α4β7.

### 4. CYP450 Enzyme Inhibition Test

The purpose of this study was to evaluate the effect of the test compound on the activity of five isoenzymes (CYP1A2, CYP2C9, CYP2D6 and CYP3A4) of human liver microsomal cytochrome P450 (CYP) by using an in vitro testing system. The specific probe substrates of CYP450 isoenzymes were incubated with human liver microsomes and test compounds of different concentrations, and reduced nicotinamide adenine dinucleotide phosphate (NADPH) was added to initiate the reaction.

After the completion of the reaction, the sample was treated and liquid chromatography-tandem mass spectrometry (LC-MS/MS) was used to quantitatively detect metabolites produced by specific substrates, changes in CYP enzyme activity were determined, and IC₅₀ value was calculated to evaluate the inhibitory potential of the test compound on each CYP enzyme subtype CYP1A2, CYP2C9, CYP2D6, or CYP3A4-M (midazolam was used as the substrate).

Conclusion: the compound of the present disclosure has no apparent inhibitory effects on each CYP enzyme subtype.

### 5. Pharmacokinetic Test in Mice

Experimental animals: male BALB/c mice, 20-25 g, 6 mice/compound. Purchased from Chengdu Ddossy Experimental Animals Co., Ltd.

Test design: on the day of the test, 6 BALB/c mice were randomly grouped according to their body weights. The animals were fasted with water available for 12 to 14 h one day before the administration, and were fed 4 h after the administration.

TABLE 2
Administration information

| | | Administration information | | | | | |
|---|---|---|---|---|---|---|---|
| | | | Administration | Administration | Administration | | |
| | Quantity | | dosage | concentration | volume | Collected | Mode of |
| Group | Male | Test compounds | (mg/kg) | (mg/mL) | (mL/kg) | samples | administration |
| G1 | 3 | Compound of | 1 | 0.2 | 5 | Plasma | Intravenously |
| G2 | 3 | the present | 10 | 1 | 10 | Plasma | Intragastrically |
| | | disclosure | | | | | |

Notes:
solvent for intravenous administration: 5% DMA + 5% Solutol + 90% Saline; solvent for intragastric administration: 5% DMSO + 95% (20% SBE-β-CD in saline)
(DMA: dimethylacetamide; Solutol: polyethylene glycol-15-hydroxystearate; Saline: physiological saline; DMSO: dimethyl sulfoxide; SBE-β-CD: sulfobutyl-β-cyclodextrin)

Before and after the administration, 0.06 mL of blood was taken from the orbit of the animals under isoflurane anesthesia and placed in an EDTAK2 centrifuge tube. The blood was centrifuged at 5000 rpm and 4° C. for 10 min to collect the plasma. The blood collection time points for the intravenous group and intragastric administration group were: 0 min, 5 min, 15 min, 30 min, 1 h, 2 h, 4 h, 6 h, 8 h, and 24 h. Before analysis and detection, all samples were stored at −80° C. The samples were analyzed quantitatively by LC-MS/MS.

### Oral Absorption Results of Test Compounds

| | CL | AUC | |
|---|---|---|---|
| Compound No. | (mL · min⁻¹ kg⁻¹) | (ng · h · mL⁻¹) | F |
| Compound 39-2 | 16.2 | 1617 | N/A |
| Compound 40-2 | 10.0 | 4104 | N/A |
| Compound 43-2 | 41.6 | 1938 | 47.1% |
| Compound 44-2 | 16.3 | 3119 | 30.3% |
| Compound 45-2 | 17.7 | 2506 | N/A |
| Control | 56.0 | 830 | 28.2% |
| compound A | | | |

Conclusion: the compounds of the present disclosure have good oral absorption properties in mice.

### 6. Pharmacokinetic Test in Rats

Experimental animals: male SD rats, about 220 g, 6-8 weeks old, 6 rats/compound. Purchased from Chengdu Ddossy Experimental Animals Co., Ltd.

Test design: on the day of the test, 6 SD rats/compound were randomly grouped according to their body weights. The animals were fasted with water available for 12 to 14 h one day before the administration, and were fed 4 h after the administration.

TABLE 3
Administration information

| | | Administration information | | | | | |
|---|---|---|---|---|---|---|---|
| | | | Administration | Administration | Administration | | |
| | Quantity | Test | dosage | concentration | volume | Collected | Mode of |
| Group | Male | compounds | (mg/kg) | (mg/mL) | (mL/kg) | samples | administration |
| G1 | 3 | Compound | 1 | 0.2 | 5 | Plasma | Intravenously |
| G2 | 3 | | 7.5 | 0.75 | 10 | Plasma | Intragastrically |
| G3 | 3 | Compound | 2.5 | 0.5 | 5 | Plasma | Intravenously |
| G4 | 3 | Compound | 10 | 1 | 10 | Plasma | Intragastrically |

Notes:
solvent for intravenous administration: 5% DMA + 5% Solutol + 90% Saline; solvent for intragastric administration: 5% DMSO + 95% (20% SBE-β-CD in saline)
(DMA: dimethylacetamide; Solutol: polyethylene glycol-15-hydroxystearate; Saline: physiological saline; DMSO: dimethyl sulfoxide; SBE-β-CD: sulfobutyl-β-cyclodextrin)

Before and after the administration, 0.10 mL of blood was taken from the orbit of the animals under isoflurane anesthesia and placed in an EDTAK2 centrifuge tube. The blood was centrifuged at 5000 rpm and 4° C. for 10 min to collect the plasma. The blood collection time points for the intravenous administration group and intragastric administration group were: 0, 5 min, 15 min, 30 min, 1 h, 2 h, 4 h, 6 h, 8 h and 24 h. Before analysis and detection, all samples were stored at −80° C., and a quantitative analysis of samples was performed using LC-MS/MS.

Conclusion: the compounds of the present disclosure have good oral absorption properties in rats.

### 7. Pharmacokinetic Test in Beagle Dogs

Experimental animals: male beagle dogs, about 8-11 kg, 5-6 beagle dogs/compound, purchased from Beijing Marshall Biotechnology Co., Ltd.

Experimental method: on the day of the test, 5-6 beagle dogs/compound were randomly grouped according to their body weights. The animals were fasted with water available for 12 to 14 h one day before the administration, and were fed 4 h after the administration.

TABLE 4
Administration information

| | | Administration information | | | | | |
|---|---|---|---|---|---|---|---|
| | | | Administration | Administration | Administration | | |
| | Quantity | Test | dosage | concentration | volume | Collected | Mode of |
| Group | Male | compounds | (mg/kg) | (mg/mL) | (mL/kg) | samples | administration |
| G1 | 3 | Test | 1 | 0.5 | 2 | Plasma | Intravenously |
| G2 | 3 | compounds | 5 | 1 | 5 | Plasma | Intragastrically |
| G3 | 2 | Test | 1 | 0.5 | 2 | Plasma | Intravenously |
| G4 | 3 | compounds | 5 | 1 | 5 | Plasma | Intragastrically |

Notes:
solvent for intravenous administration: 5% DMA + 5% Solutol + 90% Saline; solvent for intragastric administration: 5% DMSO + 95% (20% SBE-β-CD in saline)
(DMA: dimethylacetamide; Solutol: polyethylene glycol-15-hydroxystearate; Saline: physiological saline; DMSO: dimethyl sulfoxide; SBE-β-CD: sulfobutylether-β-cyclodextrin)

Before and after the administration, 1 ml of blood was taken from the jugular veins or limb veins, and placed in an EDTAK2 centrifuge tube. Centrifugation was performed at 5000 rpm at 4° C. for 10 min, and the plasma was collected. The blood collection time points for groups G1 and G2 (the intravenous group and intragastric administration group) were: 0 min, 5 min, 15 min, 30 min, 1 h, 2 h, 4 h, 6 h, 8 h, 10 h, 12 h, 24 h, 48 h, and 72 h. The blood collection time points for groups G3 and G4 (the intravenous group and intragastric administration group) were: 0 min, 5 min, 15 min, 30 min, 1 h, 2 h, 4 h, 6 h, 8 h, 10 h, 12 h, and 24 h. Before analysis and detection, all samples were stored at −80° C. The samples were analyzed quantitatively by LC-MS/MS.

Conclusion: the compounds of the present disclosure have good oral absorption properties in beagle dogs.

### 8. Pharmacokinetic Test in Monkeys

Experimental animals: male cynomolgus monkeys, 3-5 kg, 3-6 years old, 4-6 monkeys/compound. Purchased from Suzhou Xishan Biotechnology Inc.

Experimental method: on the day of the test, 4-6 monkeys/compound were randomly grouped according to their body weights. The animals were fasted with water available for 14 to 18 h one day before the administration, and were fed 4 h after the administration.

TABLE 5
Administration information

| | | Administration information | | | | | |
|---|---|---|---|---|---|---|---|
| | | | Administration | Administration | Administration | | |
| | Quantity | | dosage | concentration | volume | Collected | Mode of |
| Group | Male | Test compounds | (mg/kg) | (mg/mL) | (mL/kg) | samples | administration |
| G1 | 2 | Compound | 1 | 1 | 1 | Plasma | Intravenously |
| G2 | 2 | | 10 | 2 | 5 | Plasma | Intragastrically |
| G3 | 3 | Compound | 1 | 1 | 1 | Plasma | Intravenously |
| G4 | 3 | Compound | 10 | 2 | 5 | Plasma | Intragastrically |

Notes:
solvent for intravenous administration: 5% DMA + 5% Solutol + 90% Saline; solvent for intragastric administration: 5% DMSO + 95% (20% SBE-β-CD in saline)
(DMA: dimethylacetamide; Solutol: polyethylene glycol-15-hydroxystearate; Saline: physiological saline; DMSO: dimethyl sulfoxide; SBE-β-CD: sulfobutylether-β-cyclodextrin)
*Dosage is calculated based on free base.

Before and after the administration, 1.0 mL of blood samples were drawn from the limb veins and placed in an EDTAK2 centrifuge tube. Centrifugation was performed at 5000 rpm at 4° C. for 10 min, and the plasma was collected. The blood collection time points for the intravenous group and intragastric administration group were: 0 min, 5 min, 15 min, 30 min, 1 h, 2 h, 4 h, 6 h, 8 h, 10 h, 12 h, and 24 h. Before analysis and detection, all samples were stored at −80° C., and a quantitative analysis of samples was performed using LC-MS/MS.

Conclusion: the compounds of the present disclosure have good oral absorption properties in monkeys.

9. Test for hERG Potassium Ion Channel

Experimental platform: electrophysiological manual patch-clamp system

Cell line: Chinese hamster ovary (CHO) cell lines stably expressing hERG potassium ion channel

Experimental method: in CHO (Chinese Hamster Ovary) cells stably expressing hERG potassium channel, whole cell patch-clamp technique was used to record hERG potassium channel current at room temperature. The glass microelectrode was made of a glass electrode blank (BF150-86-10, Sutter) by a puller. The tip resistance after filling the liquid in the electrode was about 2-5 MO. The glass microelectrode can be connected to the patch-clamp amplifier by inserting the glass microelectrode into an amplifier probe. The clamping voltage and data recording were controlled and recorded by the pClamp 10 software through a computer. The sampling frequency was 10 kHz, and the filtering frequency was 2 kHz. After the whole cell records were obtained, the cells were clamped at −80 mV, and the step voltage that induced the hERG potassium current (I hERG) was depolarized from −80 mV to +20 mV for 2 s, then repolarized to −50 mV, and returned to −80 mV after 1 s. This voltage stimulation was given every 10 s, and the administration process was started after the hERG potassium current was confirmed to be stable (at least 1 minute). The compound was administered for at least 1 minute at each test concentration, and at least 2 cells (n≥2) were tested at each concentration.

Data processing: data analysis processing was carried out by using pClamp 10, GraphPad Prism 5 and Excel software. The inhibition degree of hERG potassium current (peak value of hERG tail current induced at −50 mV) at different compound concentrations was calculated by the following formula:

$$Inhibition \$ = [ 1 - ( I / Io ) ] \times 100 \%$$

wherein Inhibition % represents the percentage of inhibition of hERG potassium currents by the compound, and I and Io represent the amplitudes of hERG potassium current after administration and before administration, respectively.

Compound IC₅₀ was calculated using GraphPad Prism 5 software by fitting according to the following equation:

$$Y = Bottom + ( Top - Bottom ) / ( 1 + 10 \hat{} ( ( Log {IC}_{50} - X ) \times HillSlope ) )$$

Among the equation, X represents the Log value of the tested concentration of the test sample, Y represents the inhibition percentage at the corresponding concentration, and Bottom and Top represent the minimum and maximum inhibition percentage, respectively.

Conclusion: the compound of the present disclosure has no apparent hERG inhibitory activity.

### 10. Liver Microsomal Stability Test

In this experiment, liver microsomes of four species, including human, dog, rat, and mouse, were used as in vitro models to evaluate the metabolic stability of the test compound.

At 37° C., 1 μM of the test compound was co-incubated with microsomal protein and coenzyme NADPH. At given time points of the reaction (5 min, 10 min, 20 min, 30 min, and 60 min), the reaction was terminated by adding ice-cold acetonitrile containing an internal standard. The LC-MS/MS method was used to measure the concentration of the test compound in the sample. T_{1/2} was calculated using the natural logarithm (In) of the residual rate of the drug in the incubation system and the incubation time. In addition, the intrinsic clearance in liver microsomes Clint (mic) and the intrinsic clearance in liver Clint (Liver) were further calculated.

Conclusion: the compound of the present disclosure has good metabolic stability in liver microsomes.

1. A compound or a stereoisomer, a deuterated form, a solvate, a prodrug, a metabolite, a pharmaceutically acceptable salt or a co-crystal thereof, which compound is selected from a compound as represented by general formula (I), wherein 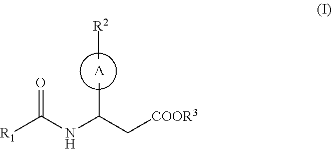
R¹ is selected from —CHR^{1a}R^{1b} or —NR^{1a}R^{1b};
R^{1a} is selected from C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
R^{1b} is selected from C₄₋₁₀ carbocyclyl or 5- to 10-membered heterocyclyl, wherein the carbocyclyl or heterocyclyl is optionally substituted with 0 to 4 R^{b}, and the heterocycle contains 1 to 4 heteroatoms selected from O, S or N;
each R^{b} is independently selected from H, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclyl, —C₀₋₄ alkyl-3- to 10-membered heterocyclyl or R^{ba}, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocycle contains 1 to 4 heteroatoms selected from O, S or N;
R^{ba} is selected from —C₀₋₄ alkyl-7- to 12-membered heterocyclyl, —C₀₋₄ alkyl-4- to 6-membered heterocyclyl connected via a carbon atom, 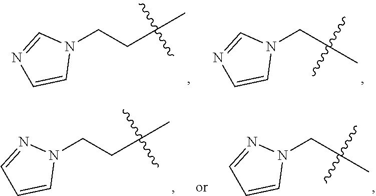 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocycle contains 1 to 4 heteroatoms selected from O, S or N;
R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclyl or —C₀₋₄ alkyl-3- to 10-membered heterocyclyl, wherein the alkyl, carbocyclyl or heterocyclyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclyl contains 1 to 4 heteroatoms selected from O, S or N;
R² is selected from C₁₋₆ alkyl, a C₆₋₁₀ aromatic ring group, a 5- to 10-membered heteroaromatic ring group, C₃₋₁₀ carbocyclyl or 5- to 10-membered heterocyclyl, and the R² is optionally substituted with 0 to 4 R^{2a}, wherein the heteroaromatic ring group and heterocyclyl contain 1 to 4 heteroatoms selected from O, S or N;
each R^{2a} is independently selected from H, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
R³ is selected from H or C₁₋₆ alkyl, wherein the alkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or a C₃₋₆ carbocycle;
ring A is selected from a 5-membered heteroaromatic ring, 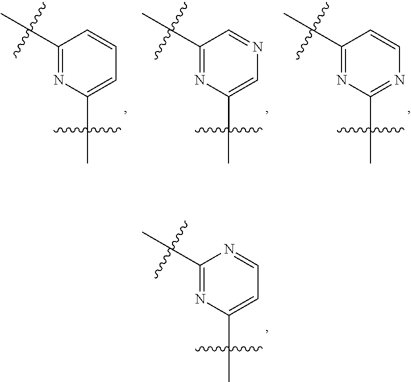 [SMILES: *c1cccc(*)n1 ; *c1cncc(*)n1 ; *c1ccnc(*)n1 ; *c1ccnc(*)n1]
or a 9- to 10-membered fused heteroaromatic ring, and the ring A is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, halogen-substituted C₁₋₆ alkyl, cyano-substituted C₁₋₆ alkyl, C₁₋₆ alkoxy or C₃₋₆ cycloalkyl, wherein the heteroaromatic ring contains 1 to 4 heteroatoms selected from N, O or S;
or ring A is selected from 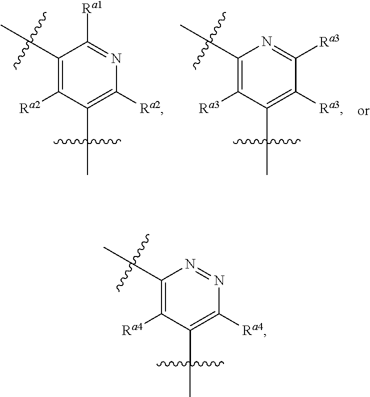
the upper left part of which is directly connected to R²;
R^{a1} is selected from H, halogen, OH, cyano, NH₂, NHC₁₋₆ alkyl, N(C₁₋₆ alkyl)₂, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, cycloalkyl or heterocyclyl is substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and at least one of the substituents is not halogen, the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a2} is independently selected from H, halogen, OH, cyano, NH₂, NHC₁₋₆ alkyl, N(C₁₋₆ alkyl)₂, C₁₋₆ alkyl, C₁₋₆ alkoxy, C₂₋₆ alkenyl, C₂₋₆ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a3} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
each R^{a4} is independently selected from H, halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S,
provided that 1) when R^{a1} is selected from H, unsubstituted C₁₋₆ alkyl, unsubstituted C₃₋₆ cycloalkyl or unsubstituted 3- to 7-membered heterocyclyl, R¹ is selected from —NR^{1a}R^{1b}, or at least one R^{a2} is not H;
2) when R¹ is selected from —CHR^{1a}R^{1b}, at least one R^{a3} is not H;
3) when R¹ is selected from —CHR^{1a}R^{1b}, at least one R^{a4} is not H;
4) the compound is not 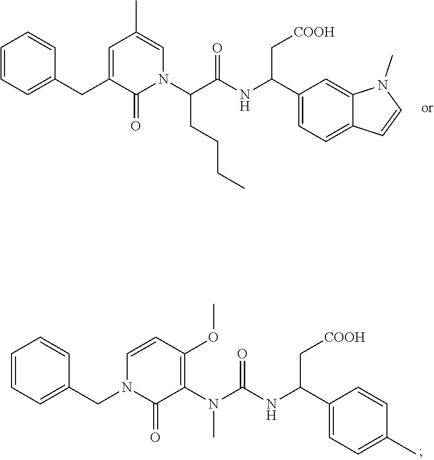 [SMILES: CCCCC(C(=O)NC(CC(=O)O)c1ccc2ccn(C)c2c1)n1cc(C)cc(Cc2ccccc2)c1=O ; COc1ccn(Cc2ccccc2)c(=O)c1N(C)C(=O)NC(CC(=O)O)c1ccc(C)cc1]
or ring A is selected from 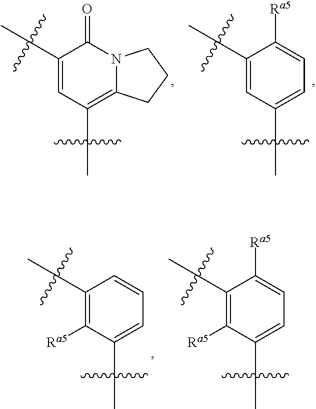
for a C₈₋₁₀ fused carbocycle, and the ring A is optionally substituted with 0 to 4 R^{a5};
or ring A is selected from 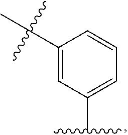 [SMILES: *c1cccc(*)c1]
and the ring A is substituted with one substituent selected from C₂₋₆ alkenyl or C₂₋₆ alkynyl and optionally substituted with 1 to 3 R^{a5};
R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S;
or when ring A is selected from 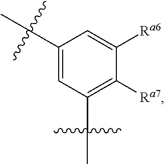
the upper left part of which is directly connected to R², one of the following conditions is met: 1) R^{1b} is selected from 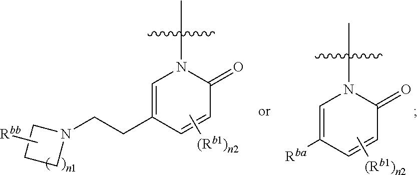
2) R² is selected from a 5- to 10-membered heteroaromatic ring or a C₆₋₁₀ aromatic ring, wherein the aromatic ring is substituted with one R^{2aa} and optionally substituted with 1 to 3 R^{2a}, and the heteroaromatic ring is optionally substituted with 1 to 4 R^{2a} and contains 1 to 4 heteroatoms selected from O, S or N;
R^{2aa} is selected from C₂₋₆ alkenyl or C₂₋₆ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
n1 is selected from 1, 2, 3 or 4;
n2 is selected from 0, 1, 2, 3 or 4;
each R^{b1} is independently selected from H, halogen, CN, C₁₋₆ alkyl, or C₃₋₆ cycloalkyl, wherein the alkyl or cycloalkyl is optionally substituted with 0 to 4 substituents selected from halogen, OH, CN, NHC₁₋₆ alkyl, N(C₁₋₆ alkyl)₂, C₁₋₄ alkyl or C₁₋₆ alkoxy;
R^{bb} is selected from OH, cyano, C₁₋₆ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy;
R^{a6} and R^{a7} are each independently selected from halogen, OH, cyano, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S.

2. The compound or the stereoisomer, deuterated form, solvate, prodrug, metabolite, pharmaceutically acceptable salt or co-crystal thereof according to claim 1, wherein,
R¹ is selected from 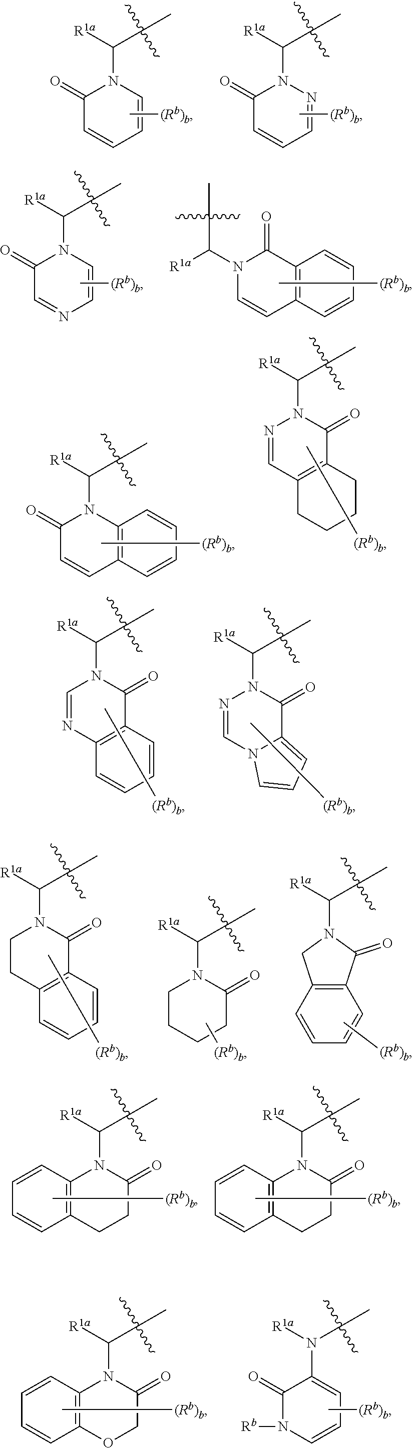 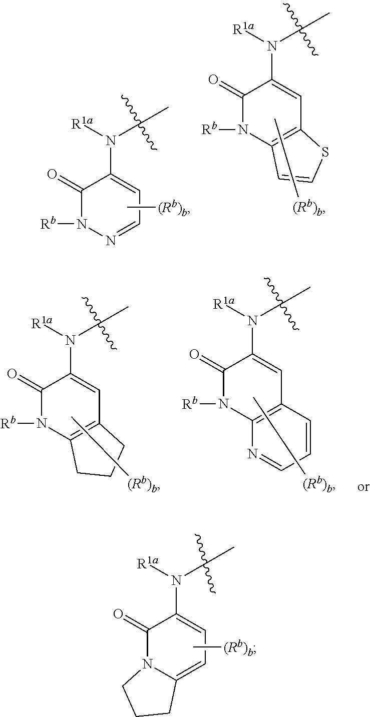
b is selected from 0, 1, 2 or 3;
each R^{b} is independently selected from H, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₂₋₄ alkynyl, C₁₋₄ alkoxy, —C₀₋₄ alkyl-C₃₋₆ carbocyclyl, —C₀₋₄ alkyl-3- to 7-membered heterocyclyl or R^{ba}, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocycle contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 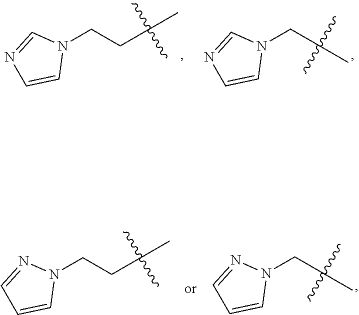 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclyl or —C₀₋₂ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
R² is selected from C₁₋₄ alkyl, a benzene ring group, a naphthalene ring group, a 5- to 6-membered heteroaromatic ring group, a 9- to 10-membered heteroaromatic ring group, C₃₋₁₀ non-aromatic carbocyclyl or 5- to 10-membered non-aromatic heterocyclyl, and the R² is optionally substituted with 0 to 4 R^{2a}, wherein the heteroaromatic ring group and heterocyclyl contain 1 to 4 heteroatoms selected from O, S or N;
each R^{2a} is independently selected from H, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
R³ is selected from H or C₁₋₄ alkyl, wherein the alkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclyl;
ring A is selected from a 5-membered heteroaromatic ring, 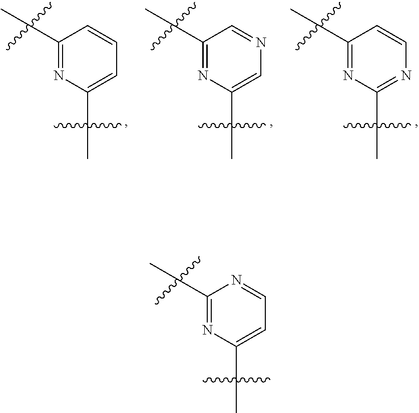 [SMILES: *c1cccc(*)n1 ; *c1cncc(*)n1 ; *c1ccnc(*)n1 ; *c1ccnc(*)n1]
or a 9- to 10-membered fused heteroaromatic ring, and the ring A is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, wherein the heteroaromatic ring contains 1 to 4 heteroatoms selected from N, O or S;
or ring A is selected from 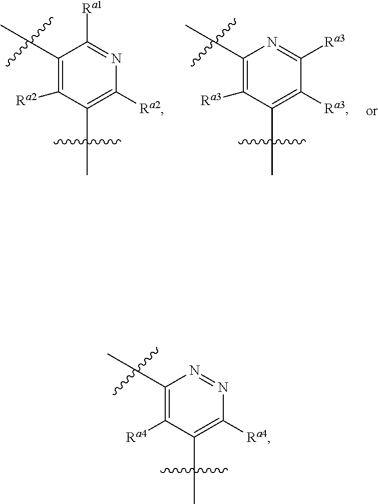
the upper left part of which is directly connected to R²;
R^{a1} is selected from H, halogen, OH, cyano, NH₂, NHC₁₋₄ alkyl, N(C₁₋₄ alkyl)₂, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a2} is independently selected from H, halogen, OH, cyano, NH₂, NHC₁₋₄ alkyl, N(C₁₋₄ alkyl)₂, C₁₋₄ alkyl, C₁₋₄ alkoxy, C₂₋₄ alkenyl, C₂₋₄ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a3} is independently selected from H, halogen, OH, cyano, C₁₋₄ alkyl, C₂₋₄ alkenyl, C₂₋₄ alkynyl, C₁₋₄ alkoxy, C₃₋₄ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a4} is independently selected from H, halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S,
provided that 1) when R^{a1} is selected from H, unsubstituted C₁₋₄ alkyl, unsubstituted C₃₋₆ cycloalkyl, or unsubstituted 3- to 7-membered heterocyclyl, R¹ is selected from 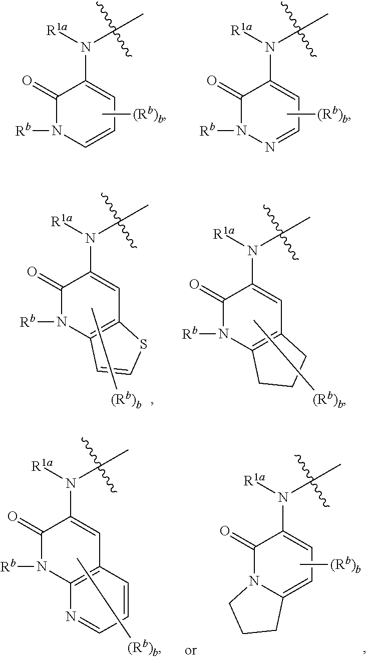
or at least one R² is not H;
2) when R¹ is selected from 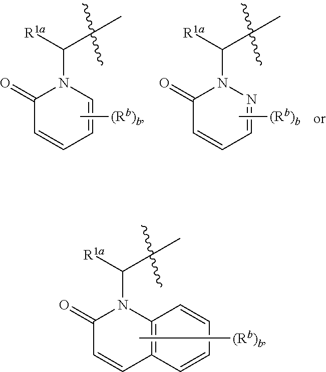
at least one R^{a3} is not H;
3) when R¹ is selected from 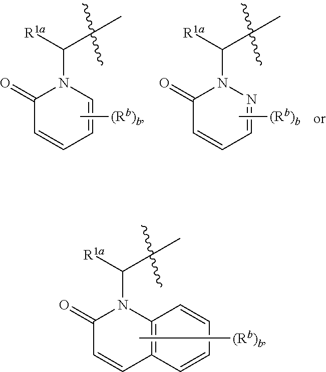
at least one R^{a4} is not H;
or ring A is selected from 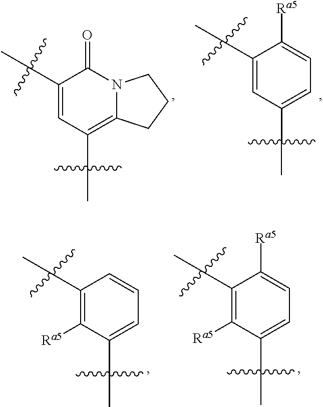
a naphthalene ring or a benzo C₄₋₆ carbocycle, and the ring A is optionally substituted with 0 to 4 R^{a5};
or ring A is selected from 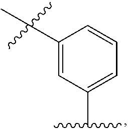 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S.

3. The compound or the stereoisomer, deuterated form, solvate, prodrug, metabolite, pharmaceutically acceptable salt or co-crystal thereof according to claim 2,
wherein each R^{b} is independently selected from H, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, C₃₋₆ cycloalkyl, 3- to 7-membered heterocycloalkyl, —CH₂—C₃₋₆ cycloalkyl, —CH₂-3- to 7-membered heterocycloalkyl, —CH₂CH₂-C₃₋₆ cycloalkyl, —CH₂CH₂-3- to 7-membered heterocycloalkyl or R^{ba}, wherein the CH₂, alkyl, alkynyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom, 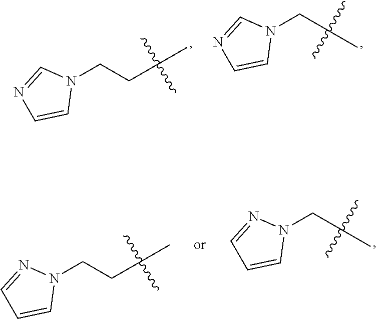 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclyl or —C₀₋₄ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
R² is selected from a benzene ring group, a naphthalene ring group, a 5- to 6-membered heteroaromatic ring group, a 9- to 10-membered heteroaromatic ring group, C₃₋₆ cycloalkyl or 3- to 7-membered heterocycloalkyl, and the R² is optionally substituted with 0 to 4 R^{2a}, wherein the heteroaromatic ring group and heterocycloalkyl contain 1 to 4 heteroatoms selected from O, S or N;
each R^{2a} is independently selected from H, halogen, OH, cyano, ═O, C₁₋₄ alkyl, C₁₋₄ alkoxy or C₃₋₆ cycloalkyl, wherein the alkyl, alkoxy or cycloalkyl is optionally substituted with 0 to 4 substituents selected from H, halogen, OH, ═O, cyano, C₁₋₄ alkyl or C₁₋₄ alkoxy;
R³ is selected from H or C₁₋₄ alkyl, wherein the alkyl is optionally substituted with 0 to 4 substituents selected from H, halogen, C₁₋₄ alkyl, C₁₋₄ alkoxy or a benzene ring group;
ring A is selected from a 5-membered heteroaromatic ring, 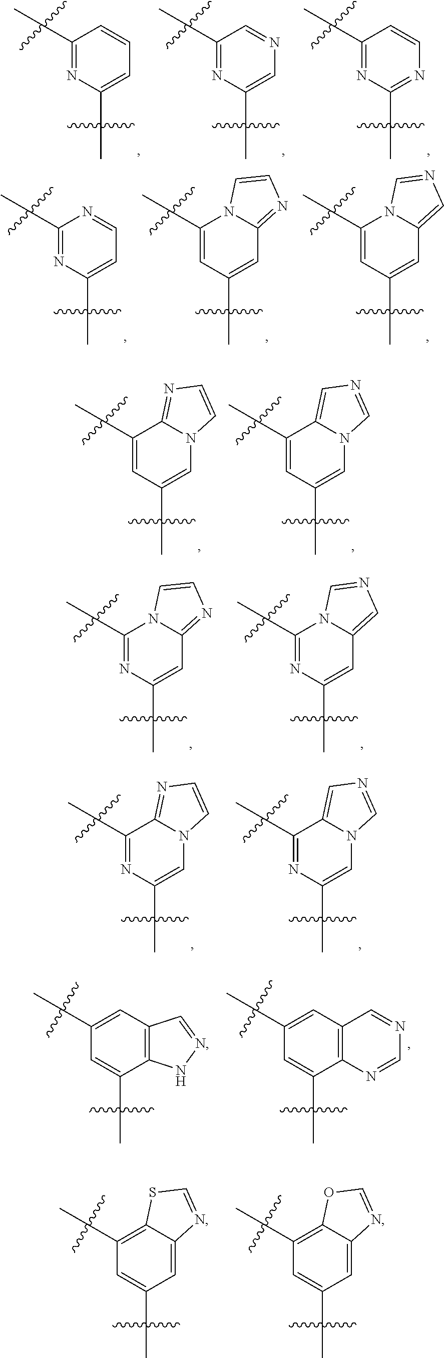 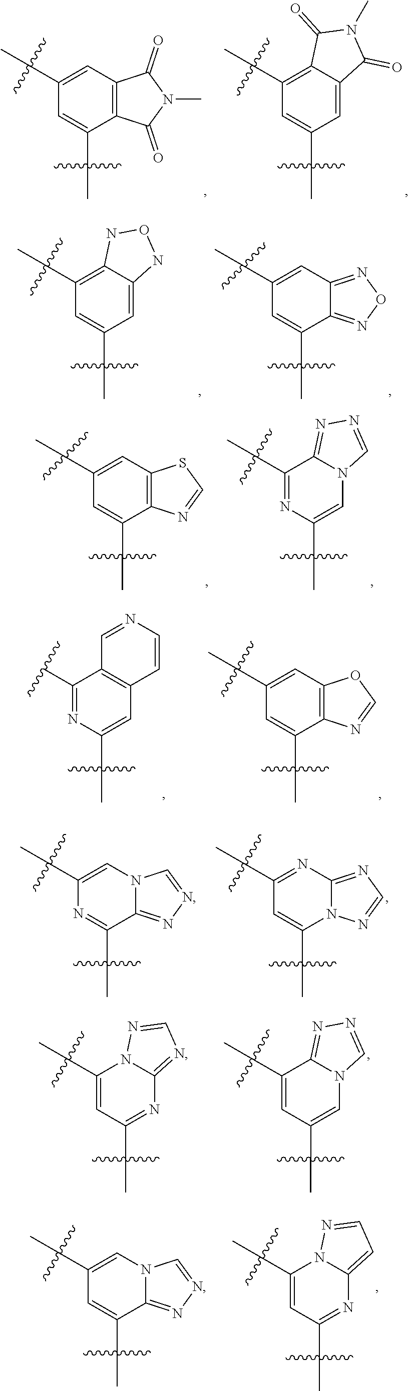 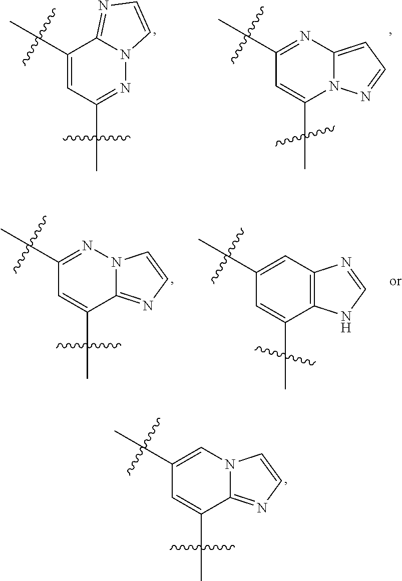 [SMILES: *c1cc(*)c2nccn2n1 ; *c1cc(*)n2nccc2n1 ; *c1cc(*)c2nccn2n1 ; *c1cc(*)c2[nH]cnc2c1 ; *c1cc(*)c2nccn2c1]
and the ring A is optionally substituted with 0 to 4 substituents selected from H, halogen, OH, cyano, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, wherein the heteroaromatic ring contains 1 to 2 heteroatoms selected from N, O or S;
or ring A is selected from 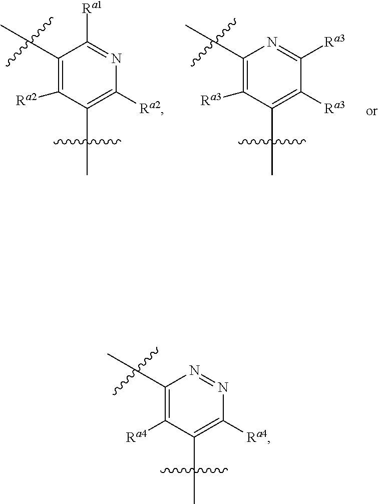
the upper left part of which is directly connected to R²;
R^{a1} is selected from H, halogen, OH, cyano, C₂₋₄ alkenyl, C₂₋₄ alkynyl or C₁₋₄ alkoxy, wherein the alkenyl, alkynyl, or alkoxy is optionally substituted with 0 to 4 substituents selected from halogen, OH, ═O, cyano, C₁₋₄ alkyl or C₁₋₄ alkoxy;
each R^{a2} is independently selected from H, halogen, OH, cyano, C₁₋₄ alkyl or C₁₋₄ alkoxy, wherein the alkyl or alkoxy is optionally substituted with 0 to 4 substituents selected from halogen, OH, ═O, cyano, C₁₋₄ alkyl or C₁₋₄ alkoxy;
each R^{a3} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy;
each R^{a4} is independently selected from H, halogen, OH, cyano, C₁₋₄ alkyl or C₁₋₄ alkoxy, wherein the alkyl or alkoxy is optionally substituted with 0 to 4 substituents selected from H, halogen, OH, ═O, cyano, C₁₋₄ alkyl or C₁₋₄ alkoxy,
provided that 1) when R^{a1} is selected from H, R¹ is selected from 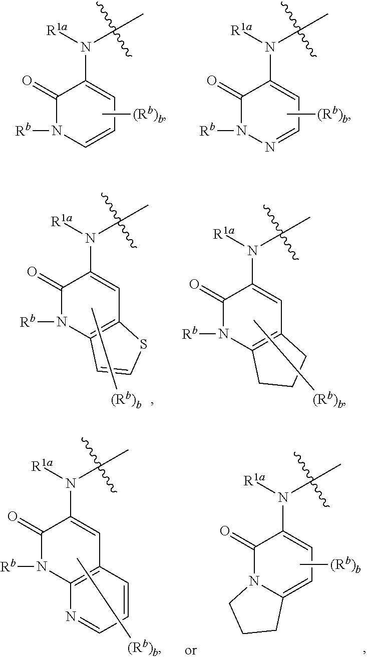
least one R^{a2} is not H;
2) when R¹ is selected from 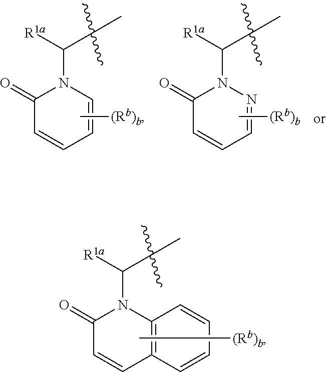
at least one R^{a3} is not H;
3) when R¹ is selected from 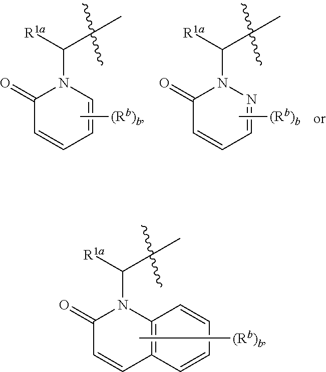
at least one R^{a4} is not H;
or ring A is selected from 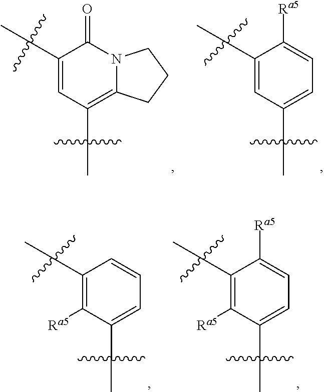
a naphthalene ring, 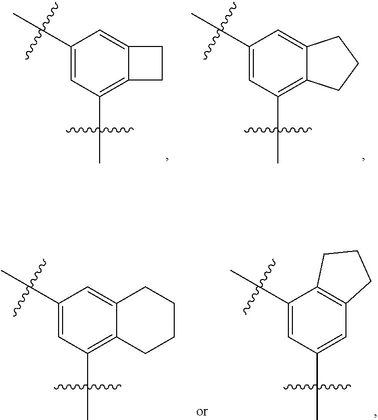 [SMILES: *c1cc(*)c2c(c1)CC2 ; *c1cc(*)c2c(c1)CCC2 ; *c1cc(*)c2c(c1)CCCC2 ; *c1cc(*)c2c(c1)CCC2]
and the ring A is optionally substituted with 0 to 4 Ras;
or ring A is selected from 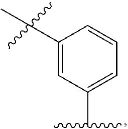 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from halogen, OH, ═O, cyano, C₁₋₄ alkyl, C₁₋₄ alkoxy or C₃₋₆ cycloalkyl.

4. The compound or the stereoisomer, deuterated form, solvate, prodrug, metabolite, pharmaceutically acceptable salt or co-crystal thereof according to claim 3,
wherein R^{1a} is selected from methyl, ethyl, propyl, butyl, isobutyl, sec-butyl, tert-butyl, —CH₂-cyclopropyl or —CH₂-cyclobutyl;
each R^{b} is independently selected from H, F, Cl, Br, I, OH, ═O, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂— cyclohexyl, —CH₂-azetidinyl, —CH₂-azacyclopentyl, —CH₂-azacyclohexyl, —CH₂-oxetanyl, —CH₂-oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 0 to 4 substituents selected from H, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{ba} is selected from one of the following optionally substituted groups: 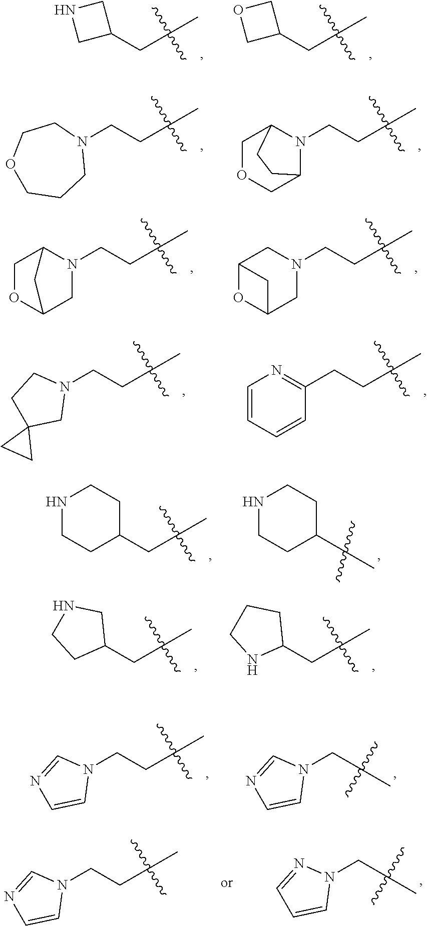
which, when substituted, is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄alkoxy or C₁₋₄ alkoxyalkyl;
R² is selected from a benzene ring group, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl or 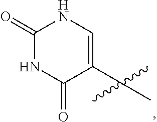 [SMILES: *c1c[nH]c(=O)[nH]c1=O]
and the R² is optionally substituted with 0 to 4 R^{2a};
each R^{2a} is independently selected from H, F, Cl, Br, I, OH, ═O, cyano, methyl, ethyl, methoxy, ethoxy, cyclopropyl or cyclobutyl, wherein the methyl, ethyl, methoxy, ethoxy, cyclopropyl or cyclobutyl is optionally substituted with 0 to 4 substituents selected from H, halogen, OH, ═O, cyano, C₁₋₄ alkyl or C₁₋₄ alkoxy;
R³ is selected from H, methyl, ethyl, propyl, butyl, isobutyl, sec-butyl, tert-butyl or benzyl;
ring A is selected from thiophene, thiazole, furan, oxazole, pyrrole, pyrazole, imidazole, 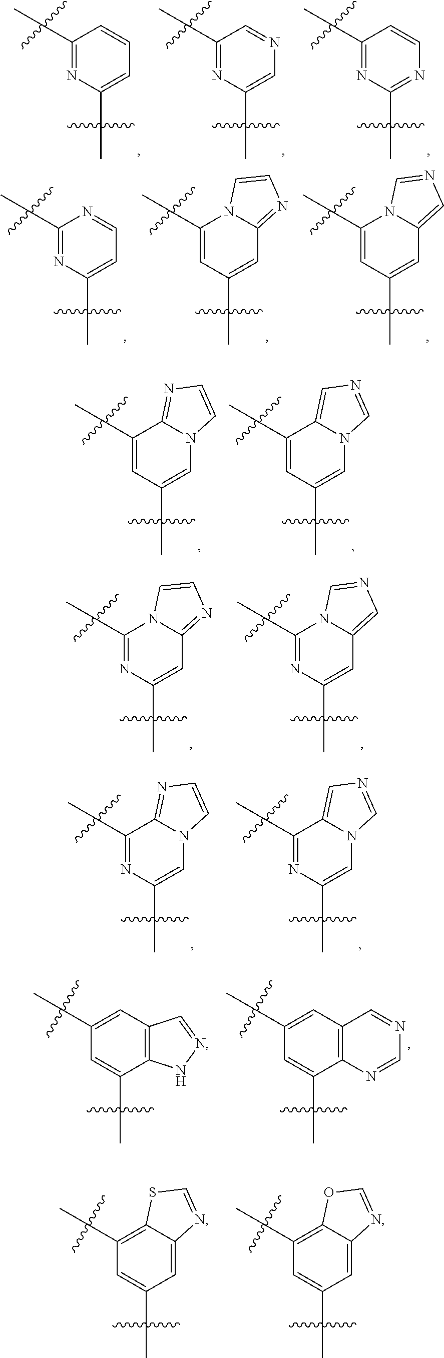 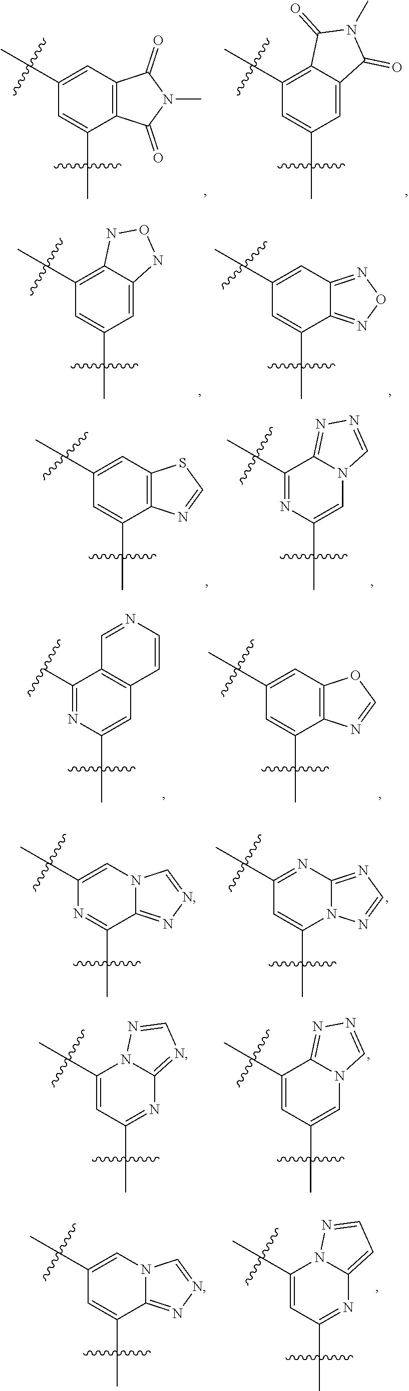 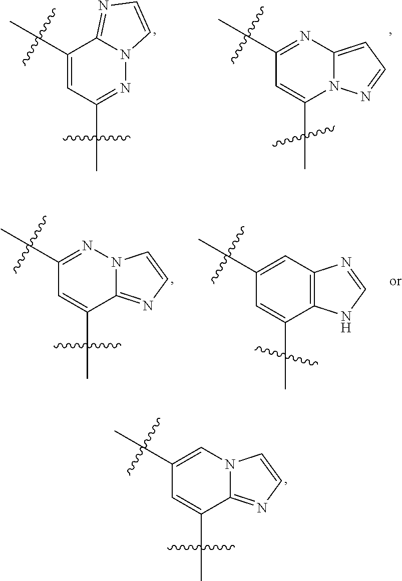 [SMILES: *c1cc(*)c2nccn2n1 ; *c1cc(*)n2nccc2n1 ; *c1cc(*)c2nccn2n1 ; *c1cc(*)c2[nH]cnc2c1 ; *c1cc(*)c2nccn2c1]
and the ring A is optionally substituted with 0 to 4 substituents selected from H, halogen, OH, cyano, C₁₋₄ alkyl, halogen-substituted C₁₋₄ alkyl, cyano-substituted C₁₋₄ alkyl, C₁₋₄ alkoxy or C₃₋₆ cycloalkyl;
or ring A is selected from 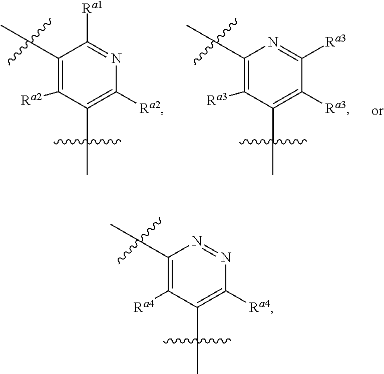
the upper left part of which is directly connected to R²;
R^{a1} is selected from H, F, Cl, Br, I, OH, cyano, ethenyl, ethynyl, methoxy or ethoxy, wherein the ethenyl, ethynyl, methoxy or ethoxy is optionally substituted with 0 to 4 substituents selected from halogen, OH, ═O, cyano, C₁₋₄ alkyl or C₁₋₄ alkoxy;
each R^{a2} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from halogen, OH, ═O, cyano, C₁₋₄ alkyl or C₁₋₄ alkoxy;
each R^{a3} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
each R^{a4} is independently selected from H, F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
or ring A is selected from 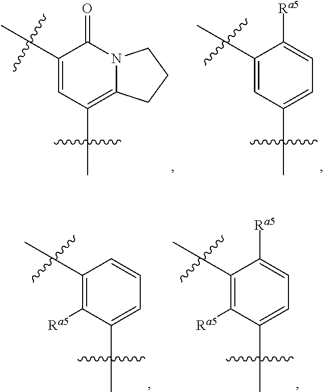
a naphthalene ring, 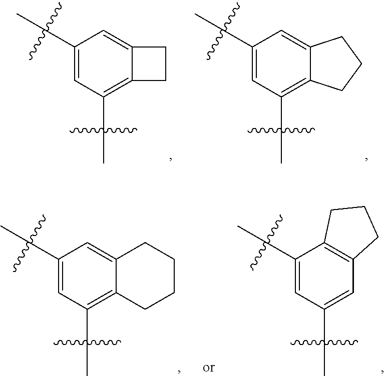 [SMILES: *c1cc(*)c2c(c1)CC2 ; *c1cc(*)c2c(c1)CCC2 ; *c1cc(*)c2c(c1)CCCC2 ; *c1cc(*)c2c(c1)CCC2]
and the ring A is optionally substituted with 0 to 4 R^{a5};
or ring A is selected from 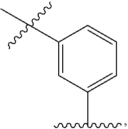 [SMILES: *c1cccc(*)c1]
and the ring A is substituted with one substituent selected from ethenyl, ethynyl, propynyl or propargyl and optionally substituted with 1 to 3 substituents selected from R^{a5};
each R^{a5} is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from halogen, OH, ═O, cyano, C₁₋₄ alkyl, C₁₋₄ alkoxy or C₃₋₆ cycloalkyl.

5. The compound or the stereoisomer, deuterated form, solvate, prodrug, metabolite, pharmaceutically acceptable salt or co-crystal thereof according to claim 4,
wherein R¹ is selected from 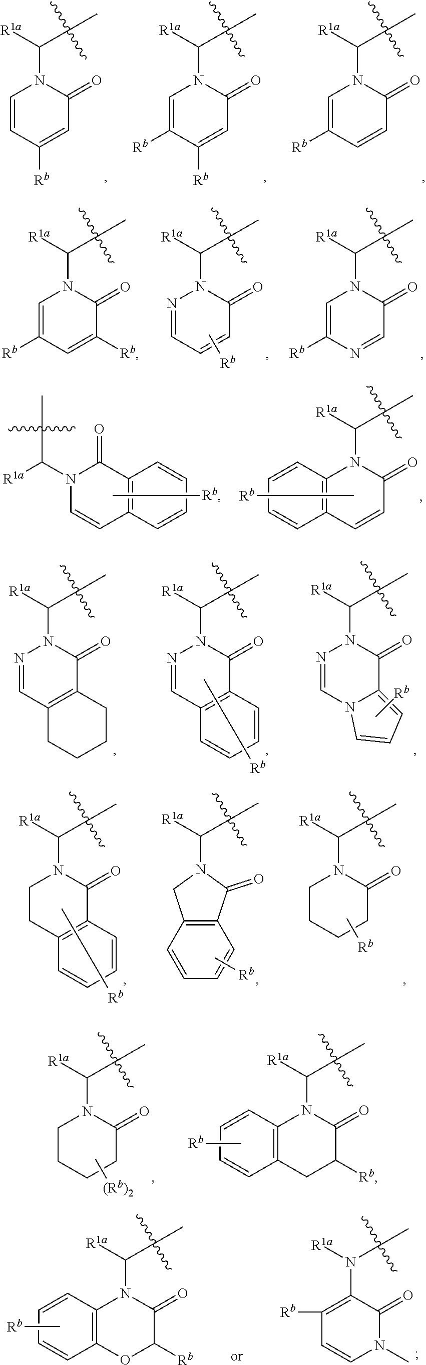
each R^{b} is independently selected from H, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azacyclopentyl, azacyclohexyl, oxetanyl, oxacyclopentyl, oxacyclohexyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂— cyclohexyl, —CH₂-azetidinyl, —CH₂-azacyclopentyl, —CH₂-azacyclohexyl, —CH₂-oxetanyl, —CH₂-oxacyclopentyl, —CH₂-oxacyclohexyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 0 to 4 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{ba} is selected from one of the following optionally substituted groups: 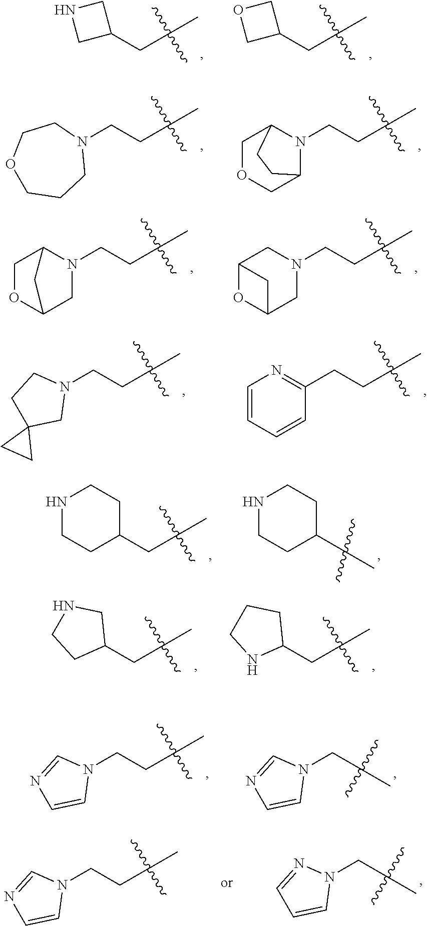
which, when substituted, is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{k} is selected from —CH₂N(CH₃)₂, —CH₂-cyclopropyl, —CH₂-cyclobutyl, cyclopropyl or cyclobutyl, wherein the cyclopropyl or cyclobutyl is optionally substituted with 1 to 4 substituents selected from F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, methoxy, ethoxy, methoxymethyl, ethoxymethyl or methoxyethyl;
R² is selected from phenyl, pyridyl, pyridonyl, azacyclopentyl, morpholinyl or 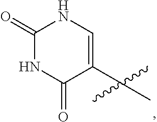 [SMILES: *c1c[nH]c(=O)[nH]c1=O]
and the R² is optionally substituted with 0 to 4 substituents selected from H, F, Cl, Br, OH, CF₃, cyano, methyl, ethyl, methoxy, ethoxy, cyclopropyl or cyclobutyl;
ring A is selected from 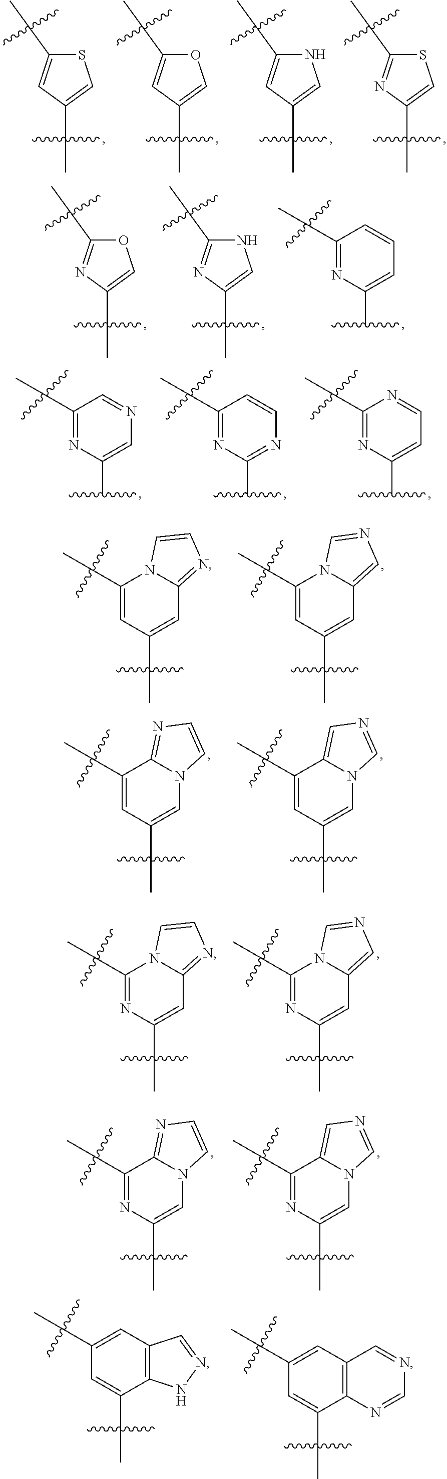 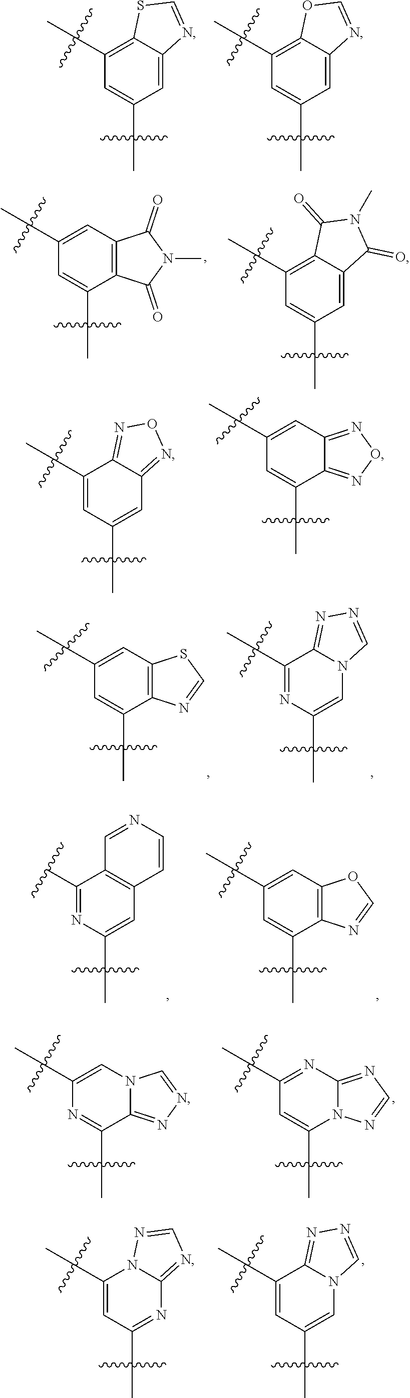 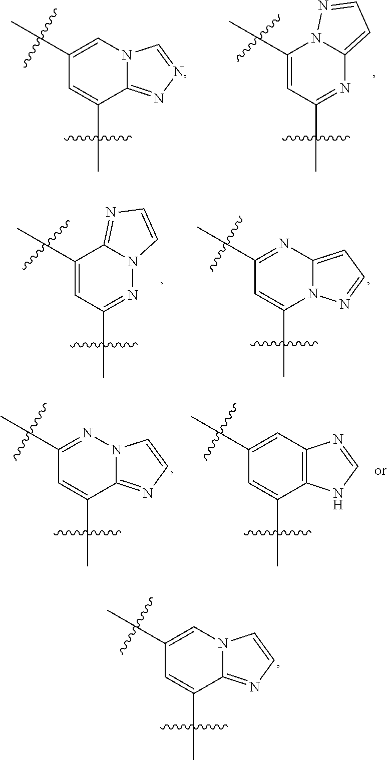
and the ring A is optionally substituted with 0 to 4 substituents selected from H, F, Cl, Br, OH, cyano, CF₃, methyl, ethyl, methyy, cyclopropyl or cyclobutyl;
or ring A is selected from 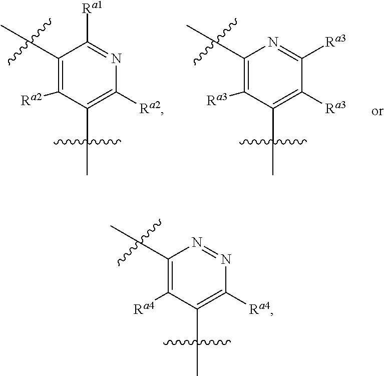
the upper left part of which is directly connected to R²,
R^{a1} is selected from H, F, Cl, Br, I, OH, cyano, ethenyl, ethynyl, methoxy, —OCF₃ or ethoxy;
each R^{a2} is independently selected from H, F, Cl, Br, I, OH, cyano, methyl, ethyl, methoxy, —OCF₃ or ethoxy;
each R^{a3} is independently selected from H, F, Cl, Br, I, OH, cyano, methyl, ethyl, methoxy, —OCF₃ or ethoxy;
each R^{a4} is independently selected from H, F, Cl, Br, I, OH, cyano, methyl, ethyl, methoxy, —OCF₃ or ethoxy;
or ring A is selected from

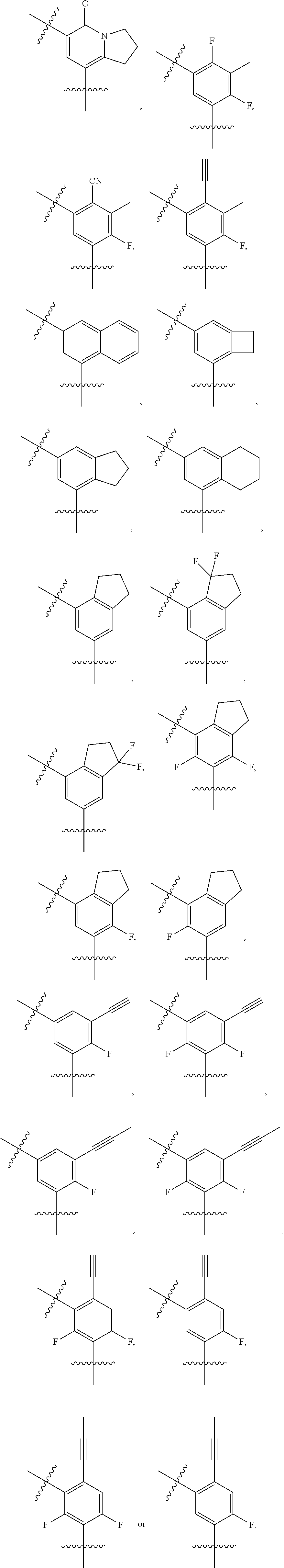

6. The compound or the stereoisomer, deuterated form, solvate, prodrug, metabolite, pharmaceutically acceptable salt or co-crystal thereof according to claim 5,
wherein R¹ is selected from 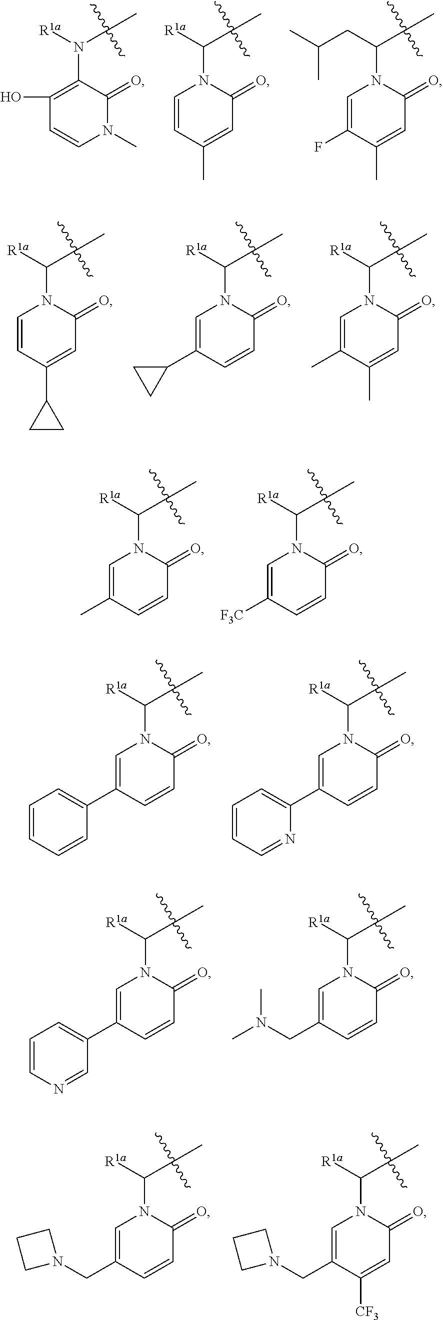 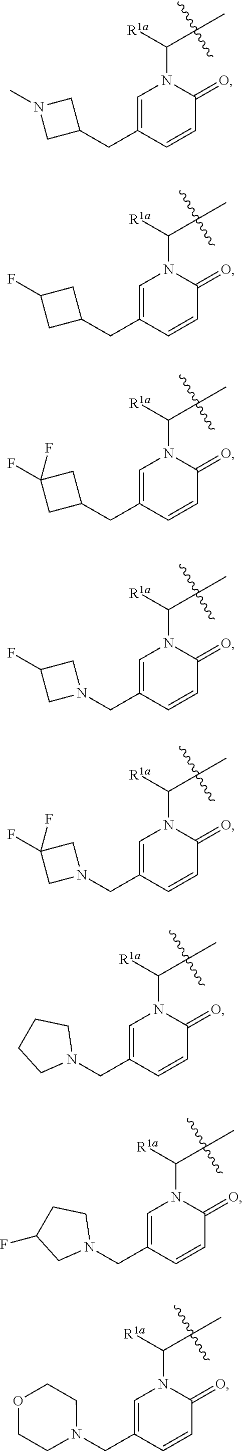 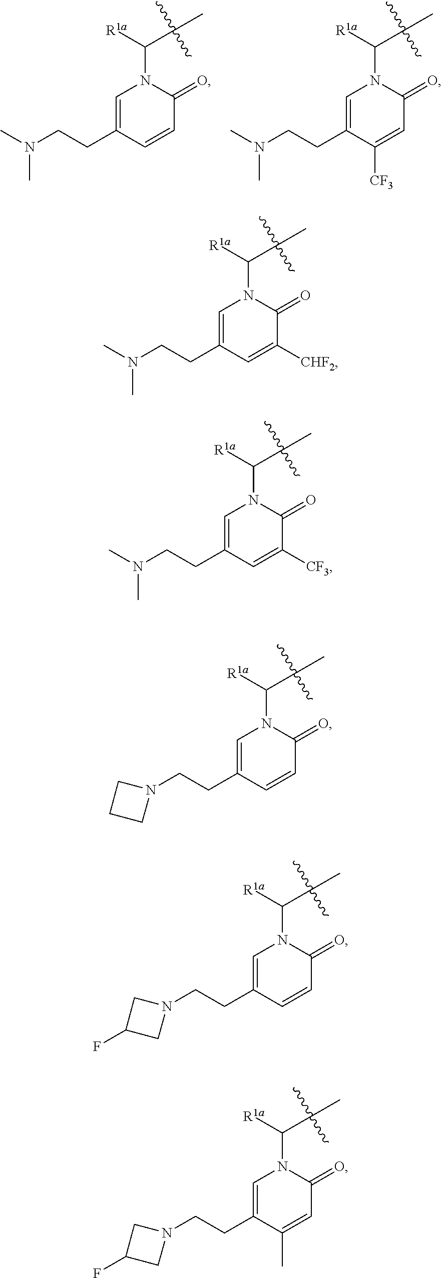 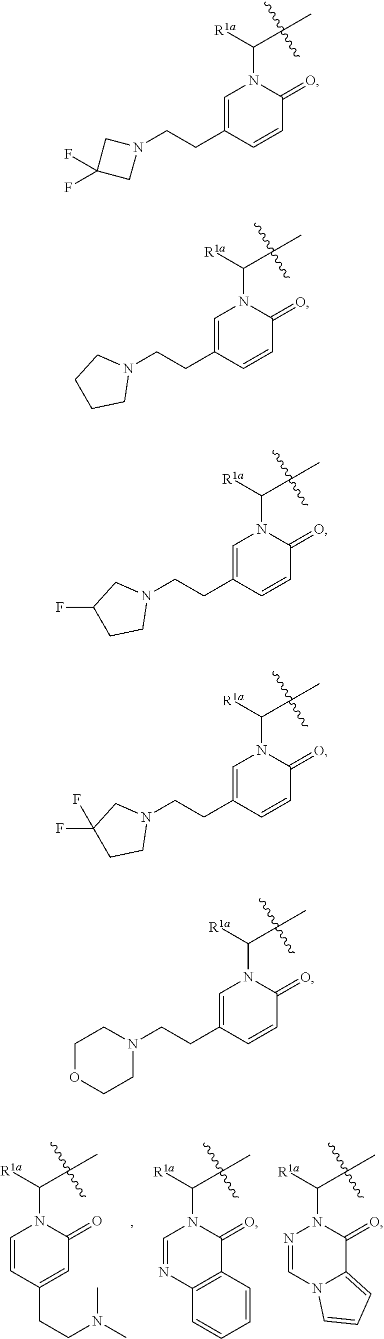 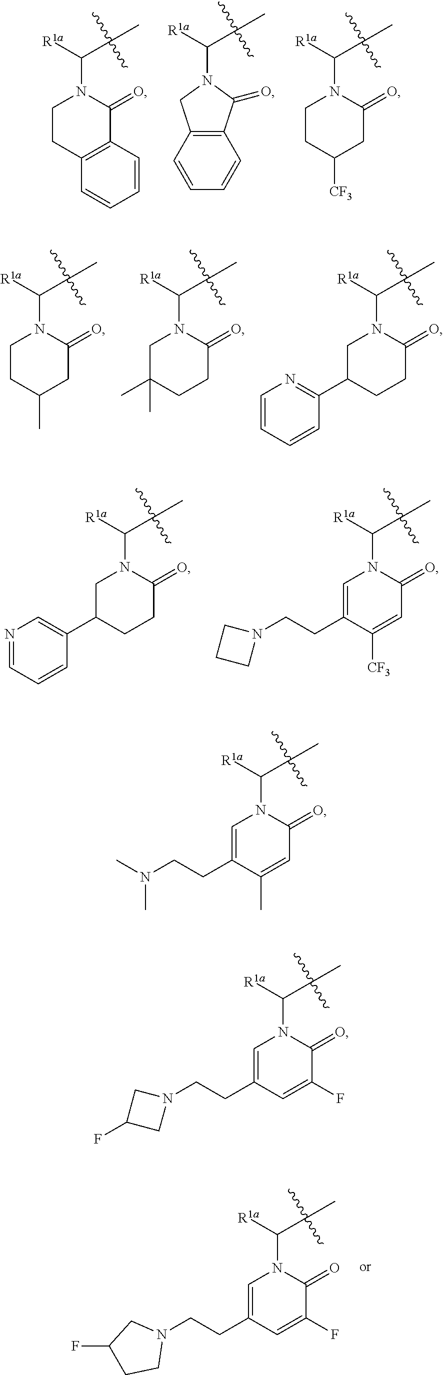 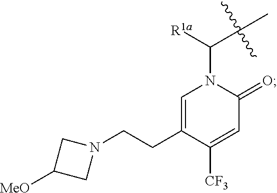
or R¹ is selected from 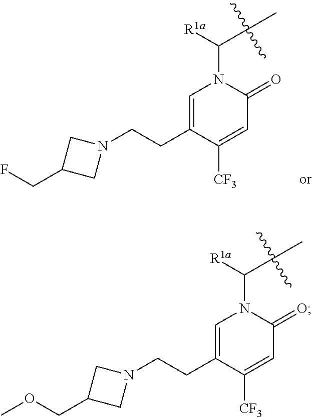
or R¹ is selected from 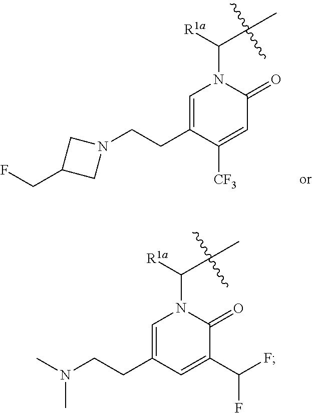
or R is selected from 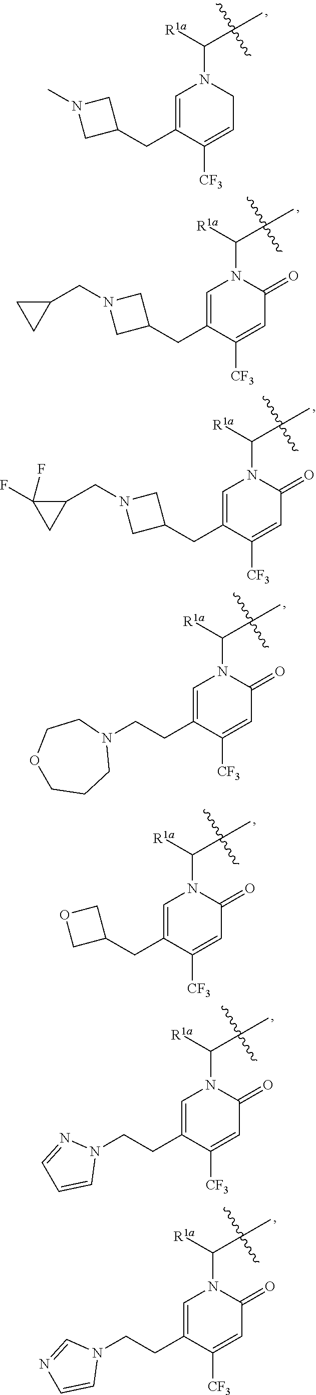 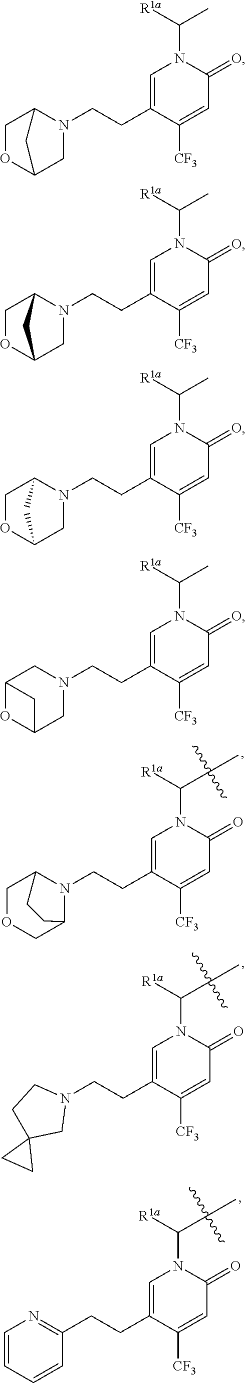 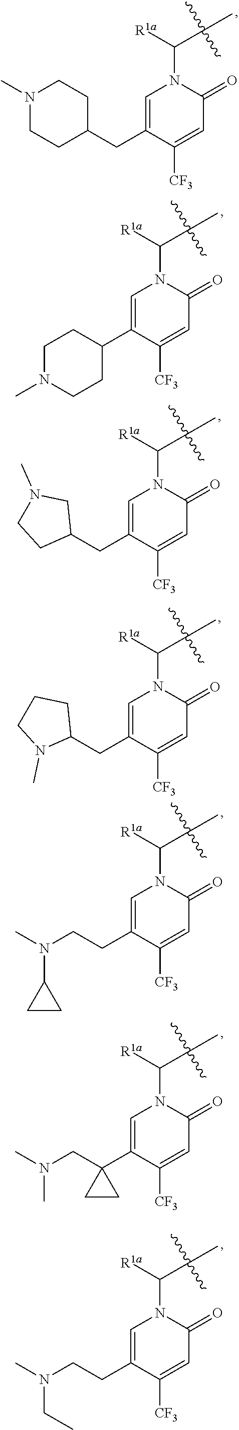 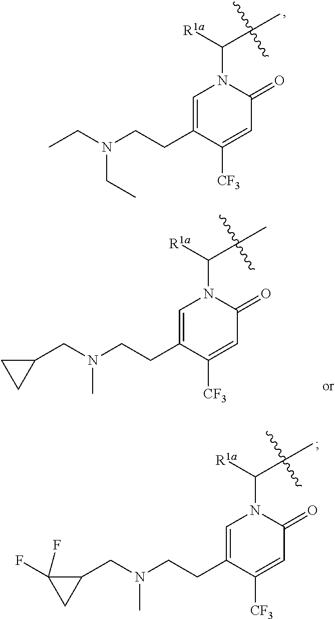
or R¹ is selected from 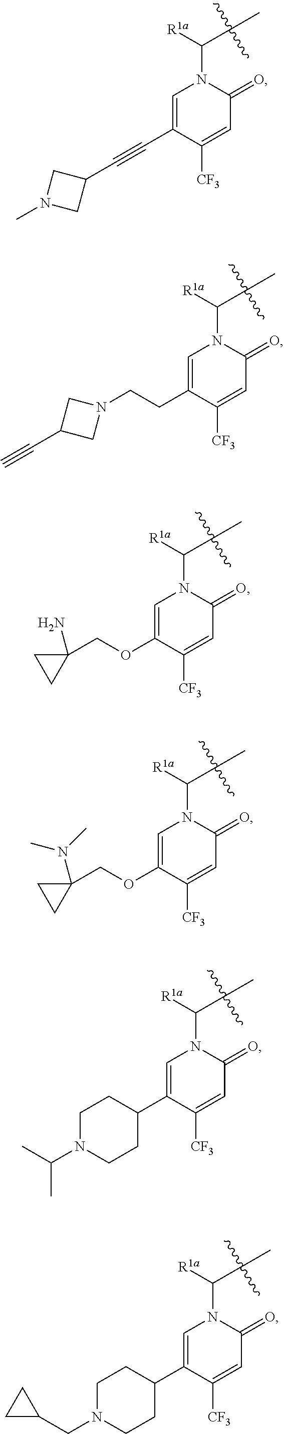 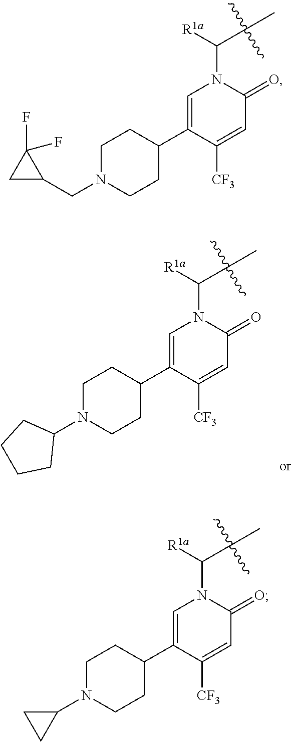
R² is selected from 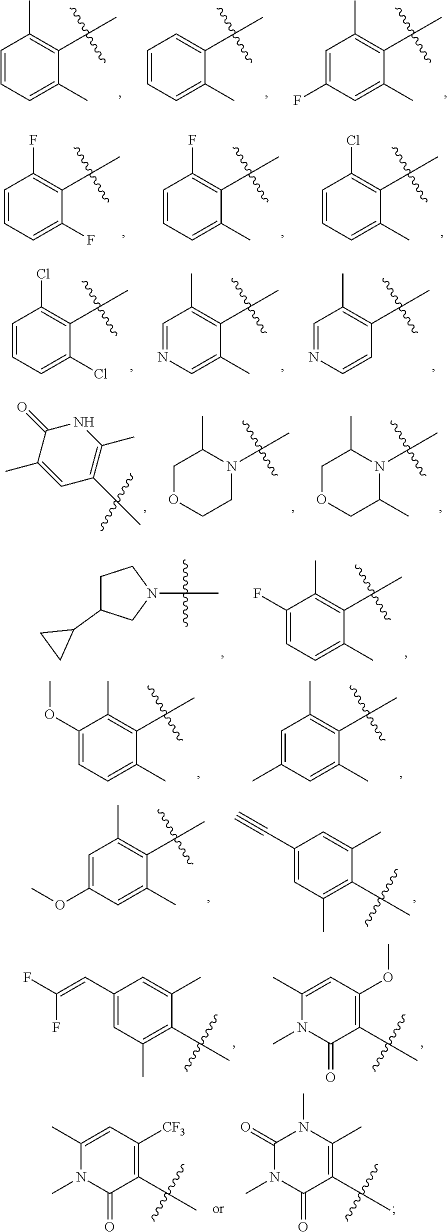
R^{1a} is selected from

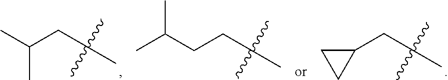

7. The compound or the stereoisomer, deuterated form, solvate, prodrug, metabolite, pharmaceutically acceptable salt or co-crystal thereof according to claim 1,
wherein ring A is selected from 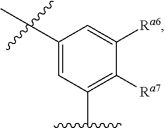
the upper left part of which is directly connected to R²;
R^{a6} and R^{a7} are each independently selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
R^{1b} is selected from 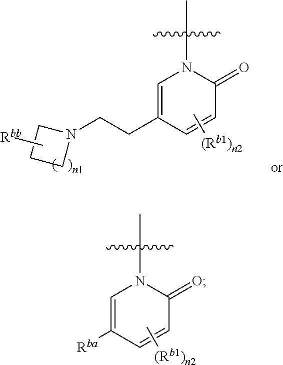
n1 is selected from 1, 2, 3 or 4;
n2 is selected from 0, 1, 2, 3 or 4;
each R^{b1} is independently selected from H, halogen, CN, C₁₋₄ alkyl or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
R^{bb} is selected from halogen, OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
R¹ is selected from 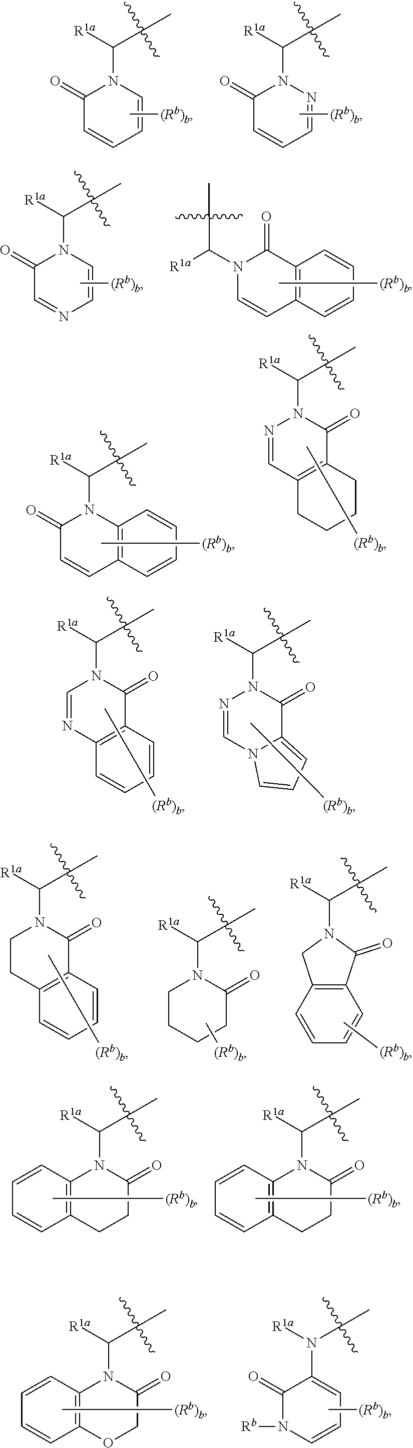 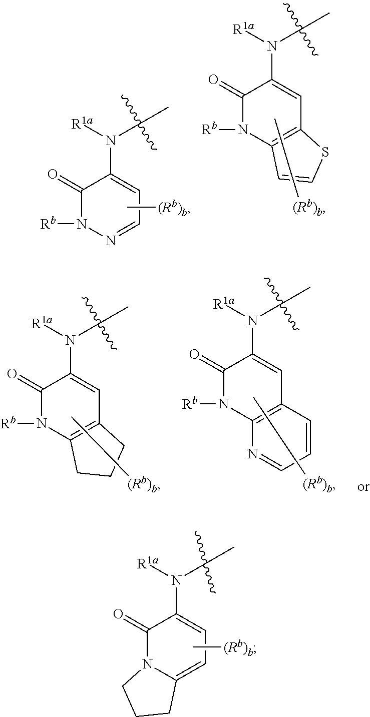
b is selected from 0, 1, 2 or 3;
each R^{b} is independently selected from H, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₂₋₄ alkynyl, C₁₋₄ alkoxy, —C₀₋₄ alkyl-C₃₋₆ carbocyclyl, —C₀₋₄ alkyl-3- to 7-membered heterocyclyl or Rha, wherein the alkyl, alkynyl, alkoxy, carbocyclyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocycle contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 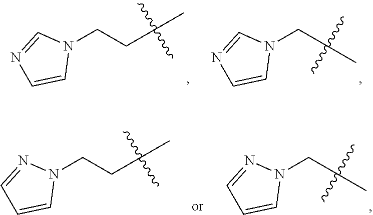 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclyl or —C₀₋₂ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N;
R² is selected from C₁₋₄ alkyl, a benzene ring group, a naphthalene ring group, a 5- to 6-membered heteroaromatic ring group, a 9- to 10-membered heteroaromatic ring group, C₃₋₁₀ non-aromatic carbocyclyl or 5- to 10-membered non-aromatic heterocyclyl, and the R² is optionally substituted with 0 to 4 R^{2a}, wherein the heteroaromatic ring group and heterocyclyl contain 1 to 4 heteroatoms selected from O, S or N;
each R^{2a} is independently selected from H, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
R³ is selected from H or C₁₋₄ alkyl, wherein the alkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclyl.

8. The compound or the stereoisomer, deuterated form, solvate, prodrug, metabolite, pharmaceutically acceptable salt or co-crystal thereof according to claim 7,
wherein R^{a6} and R^{a7} are each independently selected from F, Cl, Br, I, OH, cyano, methyl, ethyl, methoxy or ethoxy, wherein the methyl, ethyl, methoxy or ethoxy is optionally substituted with 0 to 4 substituents selected from H, halogen, OH, ═O, cyano, C₁₋₄ alkyl or C₁₋₄ alkoxy;
R^{1b} is selected from 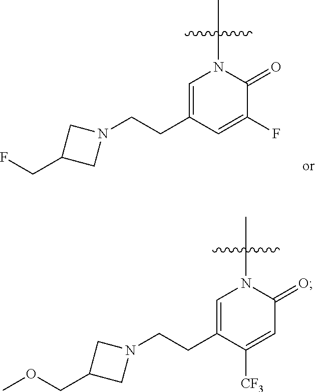
wherein each R^{b} is independently selected from H, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, C₃₋₆ cycloalkyl, 3- to 7-membered heterocycloalkyl, —CH₂—C₃₋₆ cycloalkyl, —CH₂-3- to 7-membered heterocycloalkyl, —CH₂CH₂—C₃₋₆ cycloalkyl, —CH₂CH₂-3- to 7-membered heterocycloalkyl or R^{ba}, wherein the CH₂, alkyl, alkynyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom, 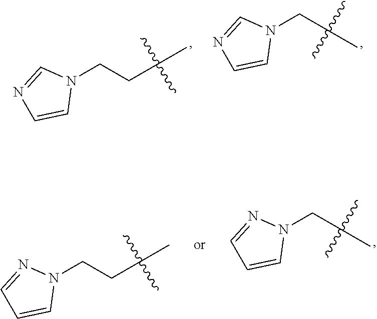 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R² is selected from a benzene ring group, a naphthalene ring group, a 5- to 6-membered heteroaromatic ring group, a 9- to 10-membered heteroaromatic ring group, C₃₋₆ cycloalkyl or 3- to 7-membered heterocycloalkyl, and the R² is optionally substituted with 0 to 4 R^{2a}, wherein the heteroaromatic ring group and heterocycloalkyl contain 1 to 4 heteroatoms selected from O, S or N;
each R^{2a} is independently selected from H, halogen, OH, cyano, ═O, C₁₋₄ alkyl, C₁₋₄ alkoxy or C₃₋₆ cycloalkyl, wherein the alkyl, alkoxy or cycloalkyl is optionally substituted with 0 to 4 substituents selected from H, halogen, OH, ═O, cyano, C₁₋₄ alkyl or C₁₋₄ alkoxy;
R³ is selected from H or C₁₋₄ alkyl, wherein the alkyl is optionally substituted with 0 to 4 substituents selected from H, halogen, C₁₋₄ alkyl, C₁₋₄ alkoxy or a benzene ring group.

9. The compound or the stereoisomer, deuterated form, solvate, prodrug, metabolite, pharmaceutically acceptable salt or co-crystal thereof according to claim 8,
wherein ring A is selected from 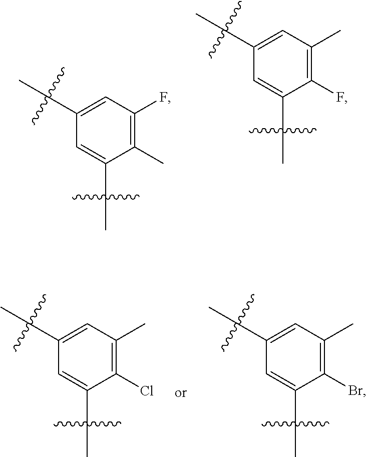
the upper left part of which is directly connected to R².

10. The compound or the stereoisomer, deuterated form, solvate, prodrug, metabolite, pharmaceutically acceptable salt or co-crystal thereof according to claim 1, which compound is selected from a compound as represented by general formula (I-a), (I-b), (I-c), (I-d) or (I-e), 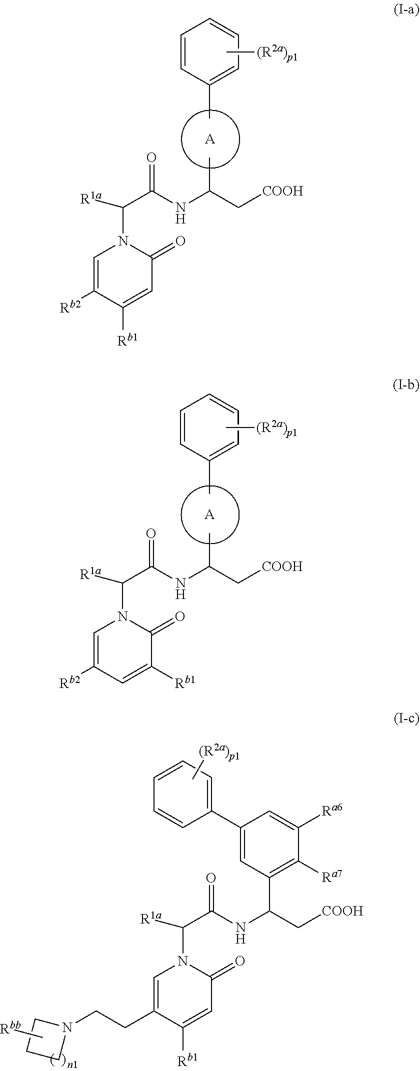 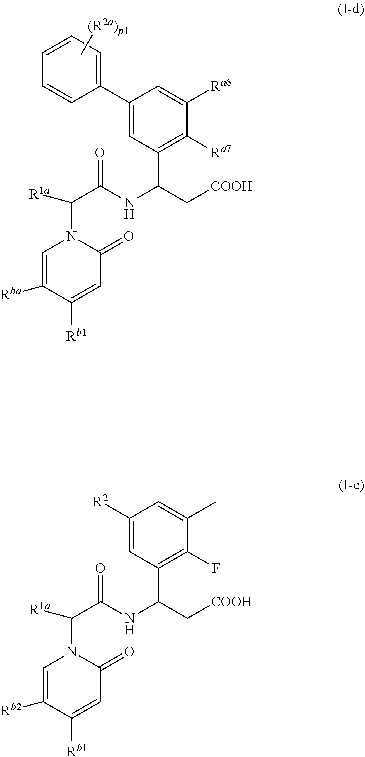
ring A is selected from a 5-membered heteroaromatic ring, 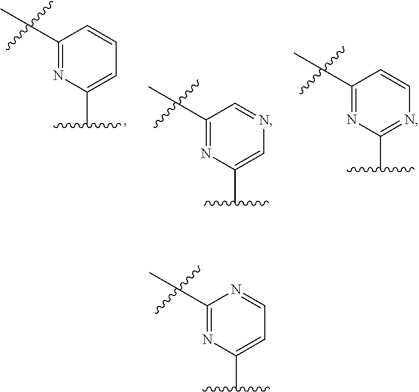 [SMILES: *c1cccc(*)n1 ; *c1cncc(*)n1 ; *c1ccnc(*)n1 ; *c1ccnc(*)n1]
or a 9- to 10-membered fused heteroaromatic ring, and the ring A is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, wherein the heteroaromatic ring contains 1 to 4 heteroatoms selected from N, O or S;
or ring A is selected from 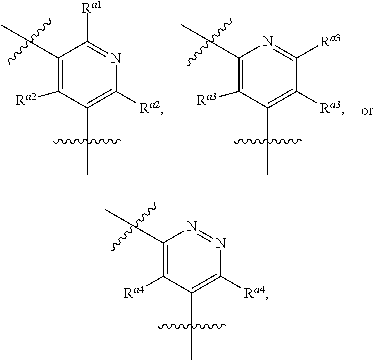
the upper left part of which is directly connected to R²;
R^{a1} is selected from H, halogen, OH, cyano, NH₂, NHC₁₋₄ alkyl, N(C₁₋₄ alkyl)₂, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a2} is independently selected from H, halogen, OH, cyano, NH₂, NHC₁₋₄ alkyl, N(C₁₋₄ alkyl)₂, C₁₋₄ alkyl, C₁₋₄ alkoxy, C₂₋₄ alkenyl, C₂₋₄ alkynyl, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a3} is independently selected from H, halogen, OH, cyano, C₁₋₄ alkyl, C₂₋₄ alkenyl, C₂₋₄ alkynyl, C₁₋₄ alkoxy, C₃₋₄ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
each R^{a4} is independently selected from H, halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S,
provided that
1) when R¹ is selected from 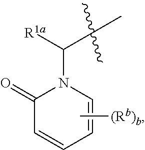
at least one R^{a3} is not H;
2) when R¹ is selected from 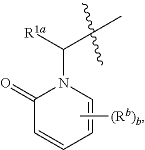
at least one R^{a4} is not H;
or ring A is selected from 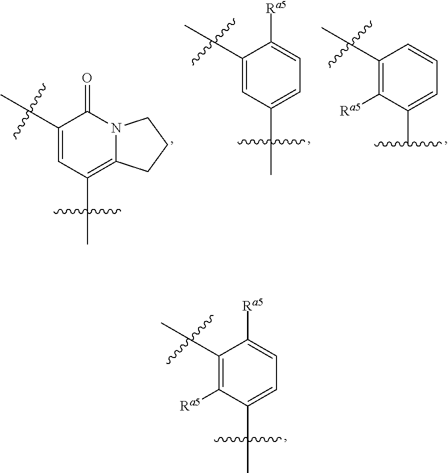
a naphthalene ring or a benzo C₄₋₆ carbocycle, and the ring A is optionally substituted with 0 to 4 R^{a5};
or ring A is selected from 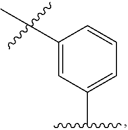 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
p1 is selected from 0, 1, 2, 3 or 4;
R^{2a} is selected from H, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from halogen, OH, CN, C₁₋₄ alkyl or C₁₋₄ alkoxy, and the heterocycloalkyl contains 1 to 4 heteroatoms selected from O, S or N;
R^{1a} is selected from 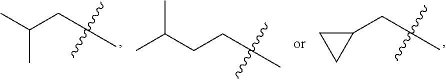
R^{b1} is selected from H, C₁₋₄ alkyl or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂-C₃₋₆ cycloalkyl or —CH₂CH₂-3- to 7-membered heterocycloalkyl, wherein the CH₂, alkyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from —C₀₋₄ alkyl-7- to 12-membered heterocyclyl, —C₀₋₄ alkyl-4- to 6-membered heterocyclyl connected via a carbon atom, 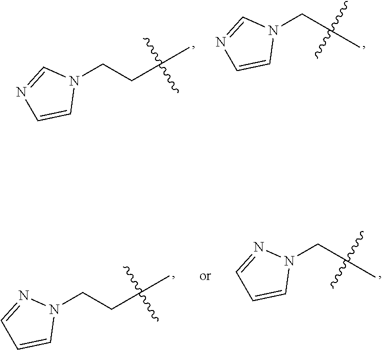 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocycle contains 1 to 4 heteroatoms selected from O, S or N;
R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclyl or —C₀₋₄ alkyl-3- to 10-membered heterocyclyl, wherein the alkyl, carbocyclyl or heterocyclyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclyl contains 1 to 4 heteroatoms selected from O, S or N;
R^{bb} is selected from OH, cyano, C₁₋₄ alkyl or C₃₋₆ cycloalkyl, wherein the alkyl or cycloalkyl is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl or C₁₋₄ alkoxy;
R^{a6} and R^{a7} are each independently selected from halogen, OH, cyano, C₁₋₄ alkyl, C₂₋₄ alkynyl or C₁₋₄ alkoxy, wherein the alkyl, alkynyl or alkoxy is optionally substituted with 0 to 4 substituents selected from H, halogen, OH, ═O, cyano, COOH, NH₂, NHC₁₋₄ alkyl, N(C₁₋₄ alkyl)₂, C₁₋₄ alkyl or C₁₋₄ alkoxy;
R² is selected from a 5- to 6-membered heteroaromatic ring group or phenyl, wherein the phenyl is substituted with one R^{2aa} and optionally substituted with 1 to 3 R^{2a}, and the heteroaromatic ring is optionally substituted with 1 to 4 R^{2a} and contains 1 to 3 heteroatoms selected from O, S or N;
R^{2aa} is selected from C₂₋₄ alkenyl or C₂₋₄ alkynyl, wherein the alkenyl or alkynyl is optionally substituted with 1 to 4 substituents selected from halogen, OH, ═O, cyano, COOH, NH₂, NHC₁₋₆ alkyl, N(C₁₋₆ alkyl)₂, C₁₋₆ alkyl or C₁₋₆ alkoxy;
n1 is selected from 1, 2 or 3.

11. The compound or the stereoisomer, deuterated form, solvate, prodrug, metabolite, pharmaceutically acceptable salt or co-crystal thereof according to claim 10,
wherein R^{2a} is selected from F, Cl, Br, I, OH, cyano, methyl, ethyl, methoxy or ethoxy;
R^{b1} is selected from H, F, CH₂F, CHF₂, CF₃, methyl or —CH₂CH₂N(CH₃)₂;
R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azacyclopentyl, —CH₂CH₂-azacyclohexyl, —CH₂CH₂-oxetanyl, —CH₂CH₂-oxacyclopentyl, —CH₂CH₂-oxacyclohexyl or —CH₂CH₂-morpholinyl, which, when substituted, is substituted with 1, 2 or 3 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{a6} and R^{a7} are each independently selected from F, Cl, Br, I, OH, cyano, methyl, ethyl, methoxy or ethoxy;
R^{bb} is selected from CH₂F, CHF₂, CF₃, methyl, ethyl or methoxymethyl;
R² is selected from

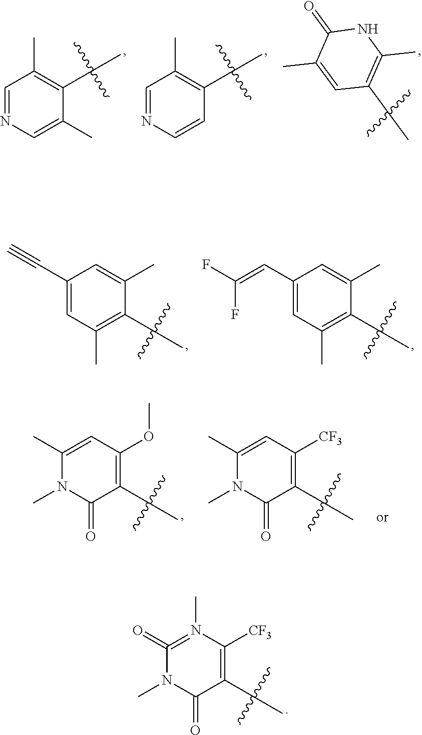

12. The compound or the stereoisomer, deuterated form, solvate, prodrug, metabolite, pharmaceutically acceptable salt or co-crystal thereof according to claim 1, which compound is selected from a compound as represented by general formula (I-f) or (I-g), 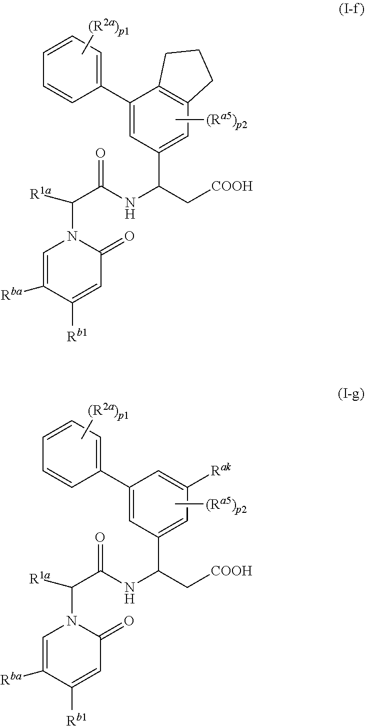
wherein p2 is selected from 0, 1 or 2;
R^{ak} is selected from C₂₋₄ alkynyl;
R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from H, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S;
p1 is selected from 0, 1, 2, 3 or 4;
R^{2a} is selected from H, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from halogen, OH, CN, C₁₋₄ alkyl or C₁₋₄ alkoxy, and the heterocycloalkyl contains 1 to 4 heteroatoms selected from 0, S or N;
R^{1a} is selected from 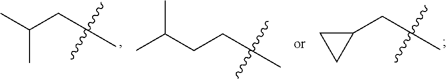
R^{b1} is selected from H, C₁₋₄ alkyl or C₃₋₆ cycloalkyl, wherein the alkyl or cycloalkyl is optionally substituted with 0 to 4 substituents selected from halogen, OH, CN, NHC₁₋₄ alkyl, N(C₁₋₄ alkyl)₂, C₁₋₄ alkyl or C₁₋₄ alkoxy;
R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl or —CH₂CH₂-3- to 7-membered heterocycloalkyl, wherein the CH₂, alkyl, cycloalkyl or heterocycloalkyl is optionally substituted with 0 to 4 substituents selected from H, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 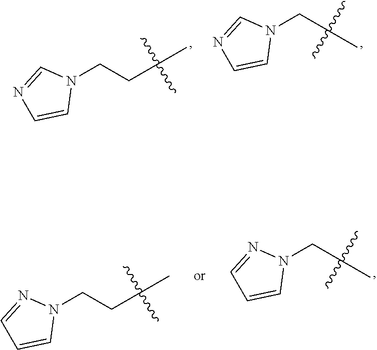 [SMILES: *CCn1ccnc1 ; *Cn1ccnc1 ; *CCn1cccn1 ; *Cn1cccn1]
and the R^{ba} is optionally substituted with 1 to 4 substituents selected from H, halogen, OH, ═O, cyano, COOH, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclyl or —C₀₋₂ alkyl-3- to 6-membered heterocyclyl, wherein the alkyl, carbocyclyl or heterocyclyl is optionally substituted with 1 to 4 substituents selected from halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocycle contains 1 to 4 heteroatoms selected from O, S or N.

13. The compound or the stereoisomer, deuterated form, solvate, prodrug, metabolite, pharmaceutically acceptable salt or co-crystal thereof according to claim 11,
R^{ak} is selected from ethynyl, propynyl or propargyl;
R^{a5} is selected from F, Cl, Br, I, OH, cyano, methyl, ethyl, methoxy, ethoxy or ethynyl.

14. The compound or the stereoisomer, deuterated form, solvate, prodrug, metabolite, pharmaceutically acceptable salt or co-crystal thereof according to claim 1, wherein the compound is selected from one of the structures shown in Table E-1 or E-2.

15. A pharmaceutical composition, comprising the compound or the stereoisomer, deuterated form, solvate, prodrug, metabolite, pharmaceutically acceptable salt or co-crystal thereof according to claim 1, and a pharmaceutically.

16. A method for treatment of diseases related to α4β7 activity or expression level, comprising administering the compound or the stereoisomer, deuterated form, solvate, prodrug, metabolite, pharmaceutically acceptable salt or co-crystal thereof according to claim 1.

17. The method according to claim 16, wherein, the diseases are selected from inflammatory bowel diseases.

18. A method for treating a disease in a mammal, the method comprising administering to a subject a therapeutically effective amount of the compound or the stereoisomer, deuterated form, solvate, prodrug, metabolite, pharmaceutically acceptable salt or co-crystal thereof according to claim 1.

19. The pharmaceutical composition according to claim 15, wherein, the pharmaceutical composition comprises 1-1500 mg of the compound or the stereoisomer, deuterated form, solvate, prodrug, metabolite, pharmaceutically acceptable salt or co-crystal thereof.

20. The method according to claim 18, wherein, the therapeutically effective amount is 1-1500 mg; the disease is inflammatory bowel diseases.
